(19) United States
(12) Patent Application Publication
(10) Pub. No.: US 2024/0124475 A1
(43) Pub. Date: Apr. 18, 2024

(54) BIFUNCTIONAL COMPOUNDS FOR DEGRADING BTK VIA UBIQUITIN PROTEOSOME PATHWAY

(71) Applicant: NURIX THERAPEUTICS, INC., San Francisco, CA (US)

(72) Inventors: Daniel W. Robbins, San Francisco, CA (US); Arthur T. SANDS, San Francisco, CA (US); Joel MCINTOSH, San Francisco, CA (US); Jeffrey MIHALIC, San Francisco, CA (US); Jeffrey WU, San Francisco, CA (US); Daisuke KATO, San Francisco, CA (US); Dahlia WEISS, San Francisco, CA (US); Ge PENG, San Francisco, CA (US)

(21) Appl. No.: 18/374,282

(22) Filed: Sep. 28, 2023

Related U.S. Application Data
(63) Continuation of application No. 17/876,433, filed on Jul. 28, 2022.
Application No. 17/876,433 is a continuation of application No. 15/734,774, filed on Dec. 3, 2020, now Pat. No. 11,479,556.
(60) Provisional application No. 62/836,398, filed on Apr. 19, 2019.
(60) Provisional application No. 62/887,812, filed on Aug. 16, 2019.
(60) Provisional application No. 62/901,984, filed on Sep. 18, 2019.
(60) Provisional application No. 62/767,819, filed on Nov. 15, 2018.
(60) Provisional application No. 62/745,786, filed on Oct. 15, 2018.

(51) Int. Cl. C07D 487/10; C07D 401/14; C07D 413/14; C07D 417/14; C07D 471/10; C07D 487/04; C07D 491/107; C07D 498/10

(52) U.S. Cl. CPC C07D 487/10; C07D 401/14; C07D 413/14; C07D 417/14; C07D 471/10; C07D 487/04; C07D 491/107; C07D 498/10

(57) ABSTRACT

The present invention relates to compounds useful for degrading BTK via a ubiquitin proteolytic pathway. The invention also provides pharmaceutically acceptable compositions comprising said compounds and methods of using the compositions in the treatment of various disease, conditions, or disorders.

### CROSS REFERENCE TO RELATED APPLICATION

This PCT application claims the benefit of U.S. provisional application No. 62/745,786, filed on Oct. 15, 2018; U.S. provisional application No. 62/767,819, filed on Nov. 15, 2018; U.S. provisional application No. 62/836,398, filed on Apr. 19, 2019; U.S. provisional application No. 62/887,812, filed on Aug. 16, 2019; and U.S. provisional application No. 62/901,984, filed on Sep. 18, 2019. Each of these documents is hereby incorporated by reference in its entirety.

### FIELD OF THE INVENTION

The present invention provides novel bifunctional compounds for proteolytically degrading targeted Bruton's tyrosine kinases (BTK) and methods for treating diseases modulated by BTK.

### BACKGROUND

B cell receptor (BCR) signaling controls B cell development, as well as mature B cell activation, signaling and survival. Mis-regulation of the BCR signaling pathway is associated with numerous disease indications involving B cell function, and targeting B cells and BCR signaling has clear therapeutic potential (Woyach, et al.; Blood. 120(6); 1175-1184. 2012). For example, depletion of B cells with monoclonal antibodies targeting CD20 has significant effects in treatment of B cell malignancies and auto-immune and inflammatory diseases (Cang, et al.; J Hematolo Oncol. 5; 64, 2012).

BTK is a member of the TEC family of kinases and is a crucial signaling hub in the BCR pathway. Mutations in BTK result in X-linked agammaglobulinaemia (XLA), in which B cell maturation is impaired, resulting in reduced immunoglobulin production (Hendriks, et al.; Expert Opin Ther Targets 15; 1002-1021, 2011). The central role of BTK in B cell signaling and function makes BTK an attractive therapeutic target for B cell malignancies as well as autoimmune and inflammatory diseases. Ibrutinib, a covalent inhibitor of BTK, has been approved to treat chronic lymphocytic leukemia (CLL), mantle cell lymphoma (MCL) and other B cell malignancies, as well as graft-versus-host disease (GvHD) (Miklos, et al.; Blood. 120(21); 2243-2250. 2017). Currently, ibrutinib and second-generation BTK inhibitors are being investigated for oncology and immune-related indications such as rheumatoid arthritis (Akinleye, et al.; J of Hematolo Oncol. 6: 59, 2013; Liu, et al.; J Pharm and Exper Ther. 338(1): 154-163. 2011; Di Paolo, et al.; Nat Chem Biol. 7(1): 41-50. 2011).

As an alternative to stoichiometric inhibition, proteolytic degradation of BTK could have dramatic consequences for B cell function by effectively blocking BCR signaling. Removal of BTK protein would eliminate BTK kinase activity as well as any protein interaction or scaffolding function of BTK. Specific degradation of BTK could be accomplished using heterobifunctional small molecules to recruit BTK to a ubiquitin ligase and thus promoting ubiquitylation and proteasomal degradation of BTK. Thalidomide derivatives, such as lenalidomide or pomalidomide, can be used to recruit potential substrates to cereblon (CRBN), a component of a ubiquitin ligase complex. This unique therapeutic approach could present a mechanism of action for interfering with BTK activity and BCR signaling that is distinct from the mechanism of stoichiometric BTK inhibition. Furthermore, this degradative approach could effectively target the C481S mutated form of BTK, which mutation has been clinically observed and confers resistance to inhibition by ibrutinib (Woyach, et al.; Blood. 120(6): 1175-1184. 2012).

Presently, there remains a need for bifunctional molecules that can induce the proteolytic degradation of BTK via a ubiquitin proteolytic pathway.

### SUMMARY OF THE INVENTION

The present invention provides bifunctional compounds that induce the proteolytic degradation of BTK via a ubiquitin proteolysis pathway.

The present invention provides a compound of Formula (A)

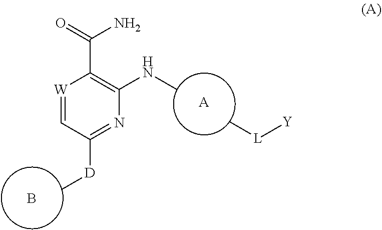

or a pharmaceutically acceptable salt thereof, wherein W is CH or N; D is a bond or —NH—; ring A is phenyl, a 9-10 membered bicyclic aryl, a 5-6 membered partially or fully unsaturated monocyclic heterocycle, or a 9-10 membered bicyclic heteroaryl, wherein the monocyclic heterocycle and bicyclic heteroaryl of ring A each possess 1-3 heteroatoms independently selected from N, O, or S, wherein ring A is optionally and independently substituted with up to 3 substituents selected from halo, —CN, —COOH, NH₂, and optionally substituted C₁₋₆ alkyl; ring B is a phenyl, a 5-6 membered heteroaryl, a 4-6 membered heterocycloalkyl, or a 8-10 membered (e.g., 8-9 membered or 9-10 membered) spiro bicyclic heterocycle, wherein ring B is optionally substituted, and wherein the heteroaryl and heterocycloalkyl of ring B has 1-3 heteroatoms independently selected from N, O, or S; L is —X¹—X²—X³—X⁴—X⁵—; X¹ is a bond, —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro or fused bicyclic heterocycloalkyl having 1-3 heteroatoms independently selected from N, O, or S, or 4-6 membered monocyclic heterocycloalkyl having 1-2 heteroatoms independently selected from N, O, or S, wherein each of the monocyclic and bicyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having 1-2 heteroatoms independently selected from N, O, or S; X³ is a bond, —C₁₋₈ alkyl-, —C≡C—, 4-6 membered cycloalkyl, —N(R)—, —N(R)—C(O)—, —(O—CH₂—CH₂)ₚ—, —(CH₂—CH₂—O)ₚ—, 4-6 membered heterocycloalkyl having 1-2 heteroatoms independently selected from N, O, or S wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, a 5-6 membered saturated, partially unsaturated, or fully unsaturated carbocycle, or a 5-6 membered saturated, partially unsaturated, or fully unsaturated heterocycle having 1-3 heteroatoms independently selected from N, O, or S; X⁵ is a bond, —C₁₋₄ alkyl-, —N(R)—, —O—, —C(O)—, or —C(O)—N(R)—; each R is independently —H or —C₁₋₃ alkyl; and each of m, n, and p is independently an integer from 1 to 3 (e.g., 1, 2, or 3); and Y is

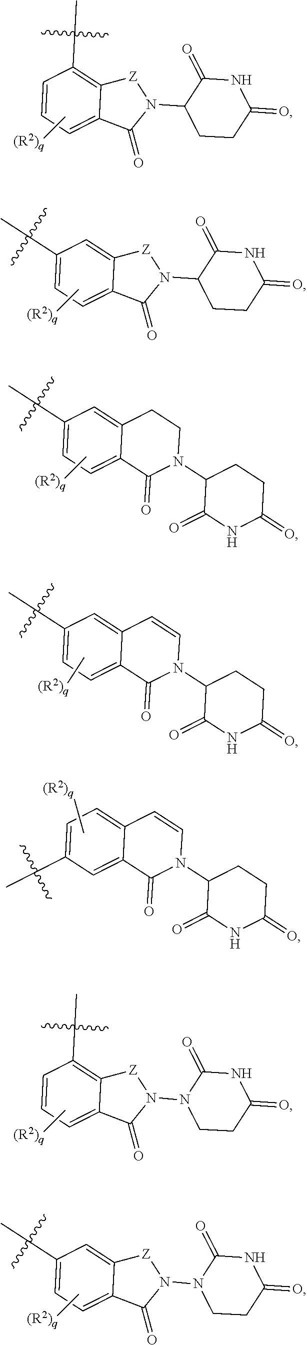

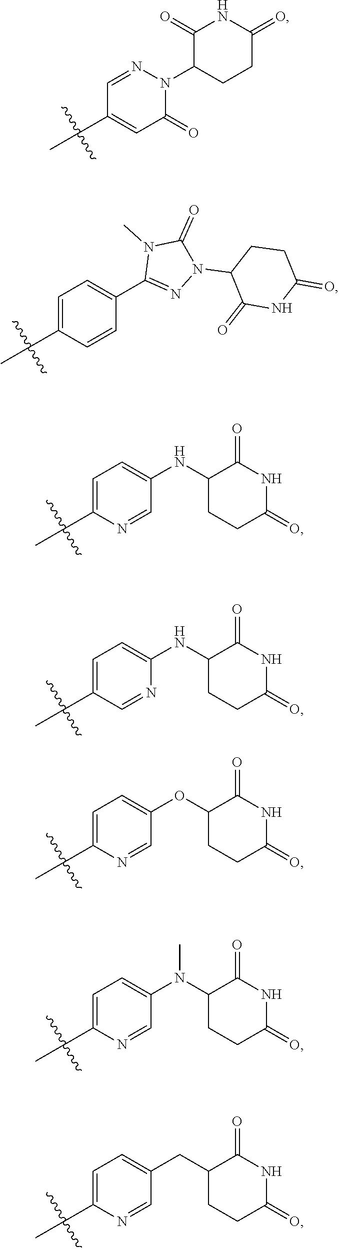

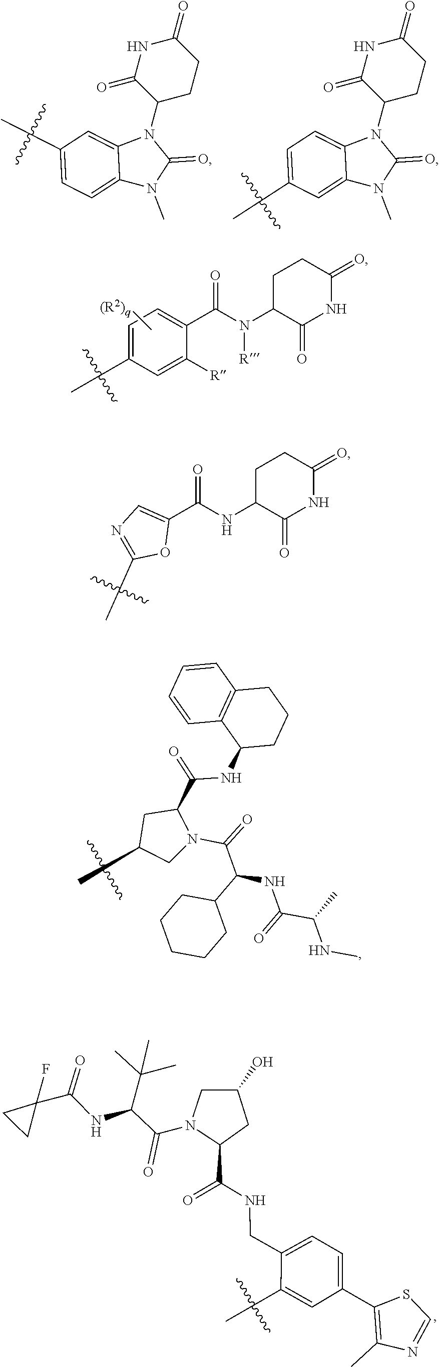

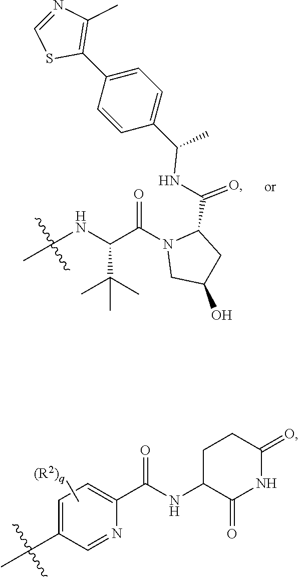

wherein each R² is independently halo, —CN, or C₁₋₄ alkyl, wherein each C₁₋₄ alkyl is optionally and independently substituted with up to three instances of halo, —CN, —COOH, —COONH₂, —NH₂, or —CF₃; each R″ and R′″ are independently H or, together with the atoms to which they are attached, form a 5-6 membered partially unsaturated or fully unsaturated benzofuzed heterocycle; each Z is —C(R^{A})₂— or —C(O)—; each R^{A} is independently —H or C₁₋₄ alkyl; and q is 0, 1, or 2.

In some embodiments, ring B is an optionally substituted 5-6 membered heterocycloalkyl having 1-2 nitrogen atoms.

In some embodiments, ring B is an optionally substituted 5-6 membered heteroaryl having 1-2 heteroatoms independently selected from N and S.

In some embodiments, ring B is

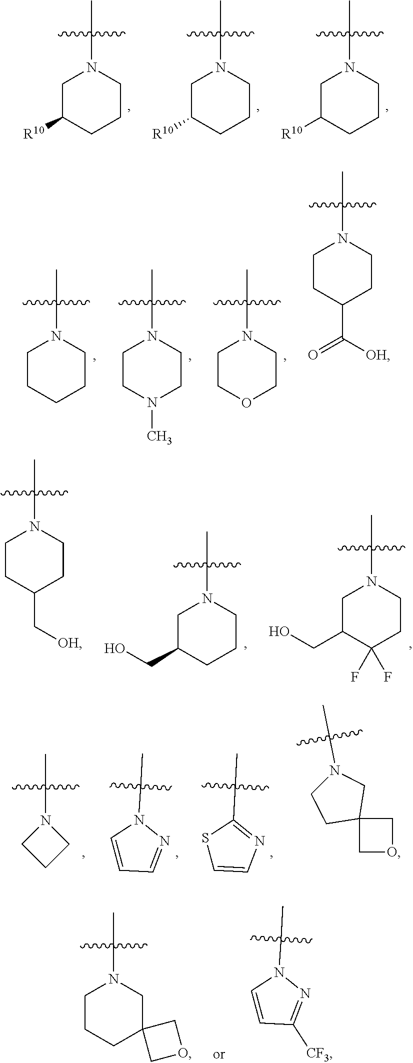

wherein R¹⁰ is

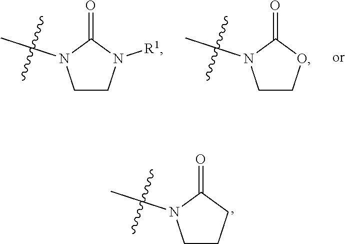

and wherein R¹ is a C₁₋₄ alkyl group. For example, ring B is

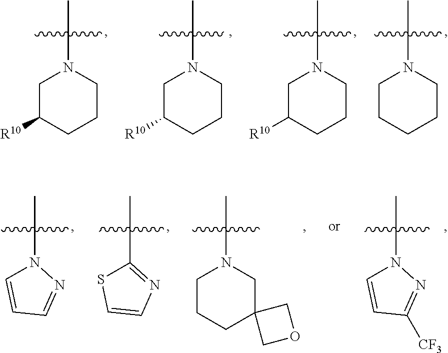

wherein R¹⁰ is

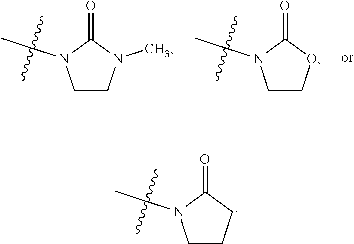 [SMILES: *N1CCN(C)C1=O ; *N1CCOC1=O ; "or" ; *N1CCCC1=O]

And, in some instances, ring B is

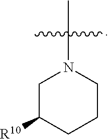

In other instances, R¹⁰ is

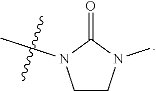 [SMILES: *N1CCN(C)C1=O]

In some embodiments, ring A

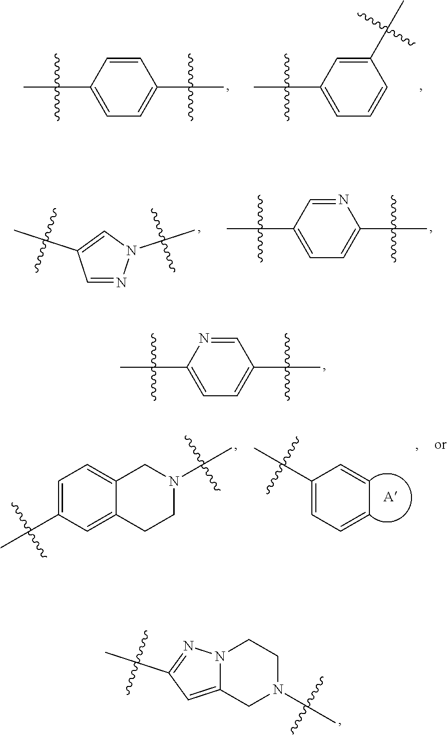

wherein ring A′ together with the phenyl ring to which it is fused form a 9-10 membered bicyclic aryl or a 9-10 membered bicyclic heteroaryl wherein the bicyclic heteroaryl (i.e., the bicyclic heteroaryl including ring A′) has 1-3 heteroatoms independently selected from N, O, or S. For example, ring A is

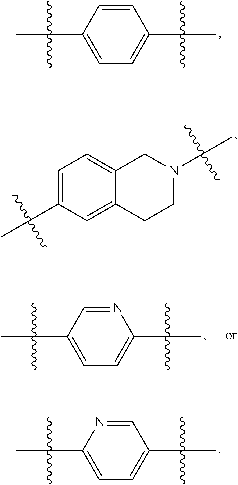 [SMILES: *c1ccc(*)cc1 ; *c1ccc2c(c1)CCN(*)C2 ; *c1ccc(*)nc1 ; *c1ccc(*)nc1]

In some embodiments, at least one of X¹, X², and X⁵ is —N(R)—, —C(O)—N(R)—, or —CH₂—.

In some embodiments, X¹ is —C(O)—N(R)—.

In some embodiments, X² is —(O—CH₂—CH₂)ₙ—, —(CH₂—CH₂—O)ₙ—, or —C₁₋₅ alkyl-.

In some embodiments, X³ is a bond, —C≡C—, —C₁₋₄ alkyl-, or —N(R)—.

In some embodiments, X⁴ is a bond, —CH₂—, or —N(R)—.

In some embodiments, X⁵ is a bond.

In some embodiments, X¹ is —(O—CH₂—CH₂—CH₂)ₘ—, m is 1, and X² is —C(O)—N(R)—.

In some embodiments, X¹ is —CH₂—, —C(O)—,

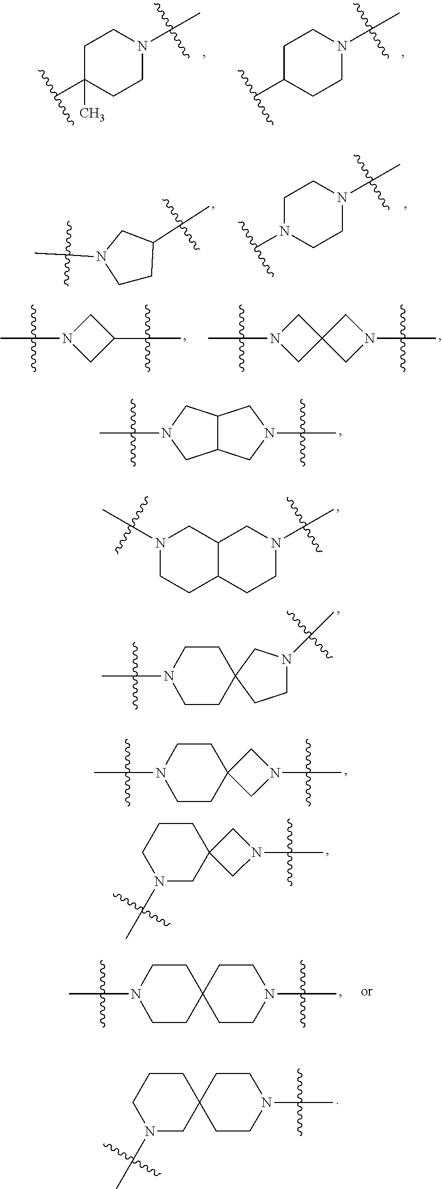

In some embodiments, X² is a bond, —C(O)—, —C₁₋₅ alkyl-,

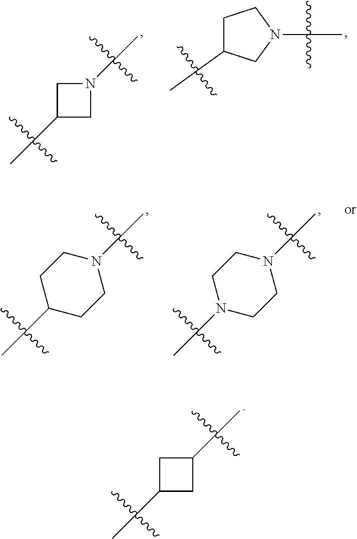 [SMILES: *C1CN(*)C1 ; *C1CCN(*)C1 ; *C1CCN(*)CC1 ; *N1CCN(*)CC1 ; *C1CC(*)C1]

In some embodiments, X³ is bond, —C₁₋₄ alkyl-, 4-6 membered cycloalkyl, or —N(R)—.

In some embodiments, X³ is a bond, —C₁₋₄ alkyl-, —NH—,

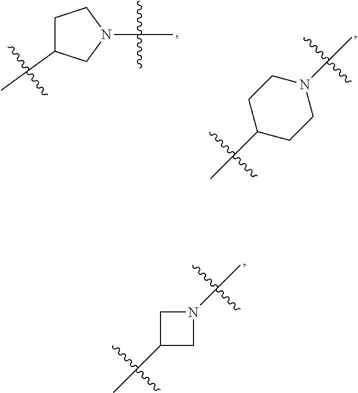 [SMILES: *C1CCN(*)C1 ; *C1CCN(*)CC1 ; *C1CN(*)C1]

### or —C≡C—.

In some embodiments, X⁴ is a bond,

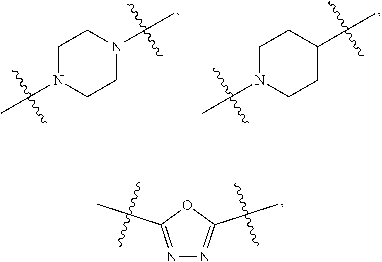 [SMILES: *N1CCN(*)CC1 ; *C1CCN(*)CC1 ; *c1nnc(*)o1]

—C₁₋₄ alkyl-, —CH₂—CH₂—N(R)—, or —N(R)—.

In some embodiments, X⁵ is a bond, —C₁₋₄ alkyl-, —N(R)—, or —C(O)—N(R)—.

In some embodiments, L is

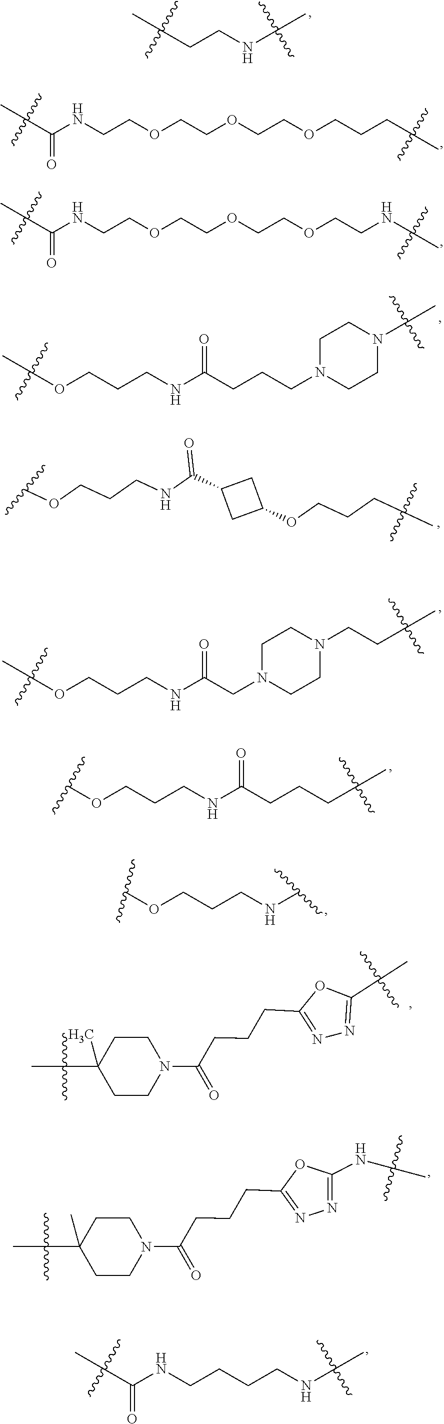

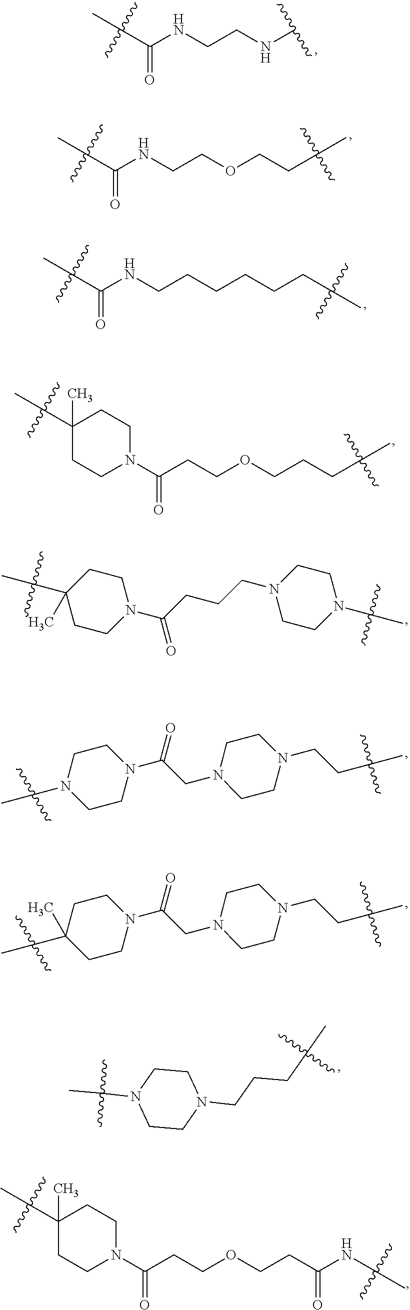

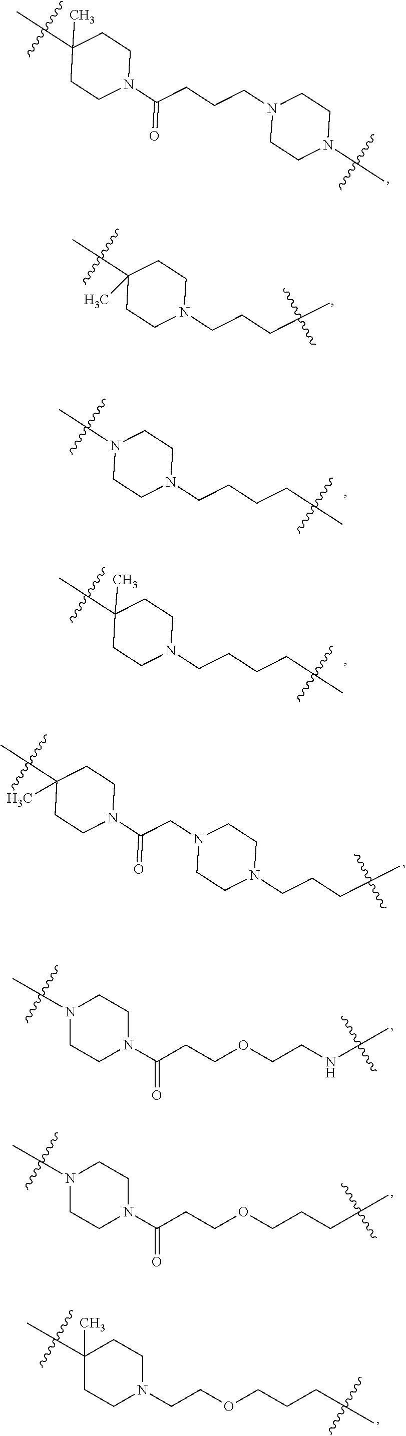

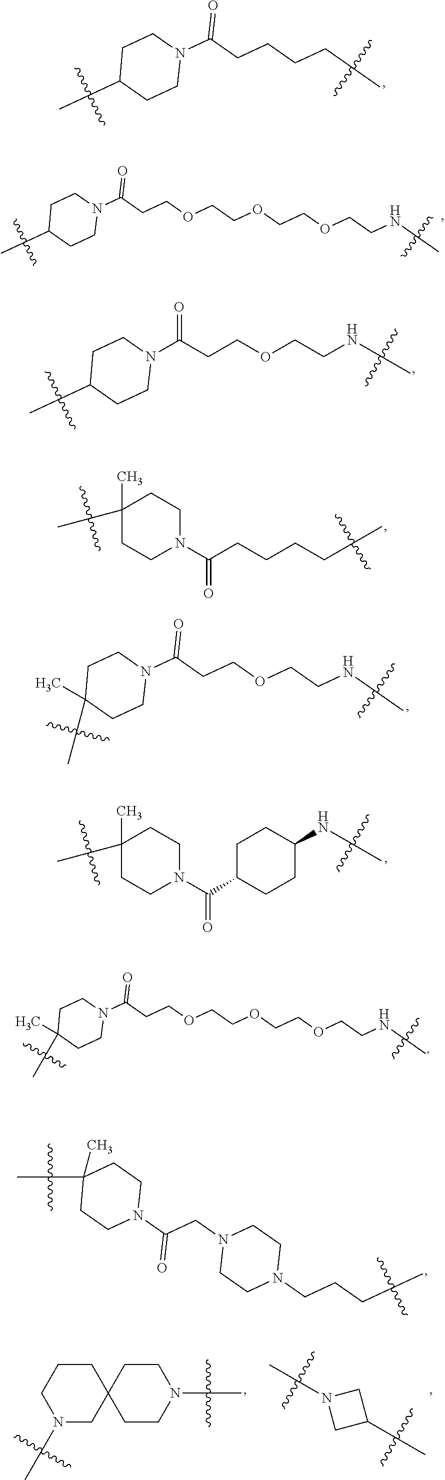

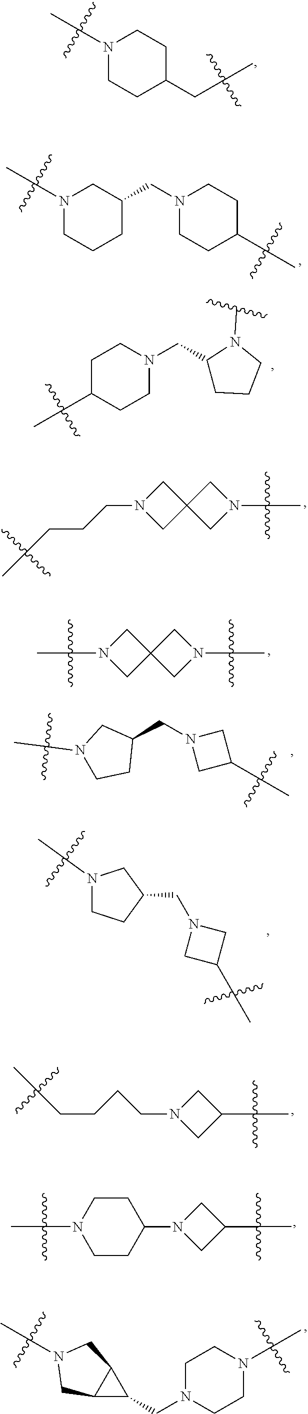

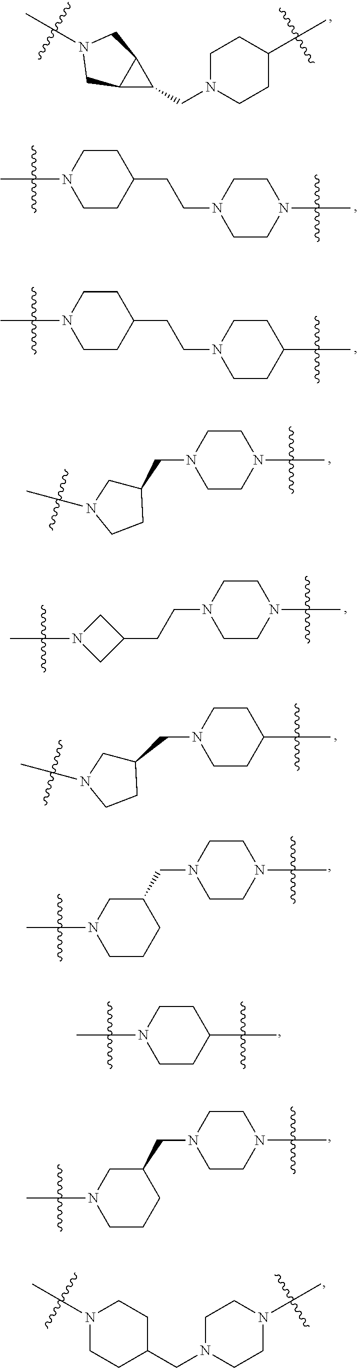

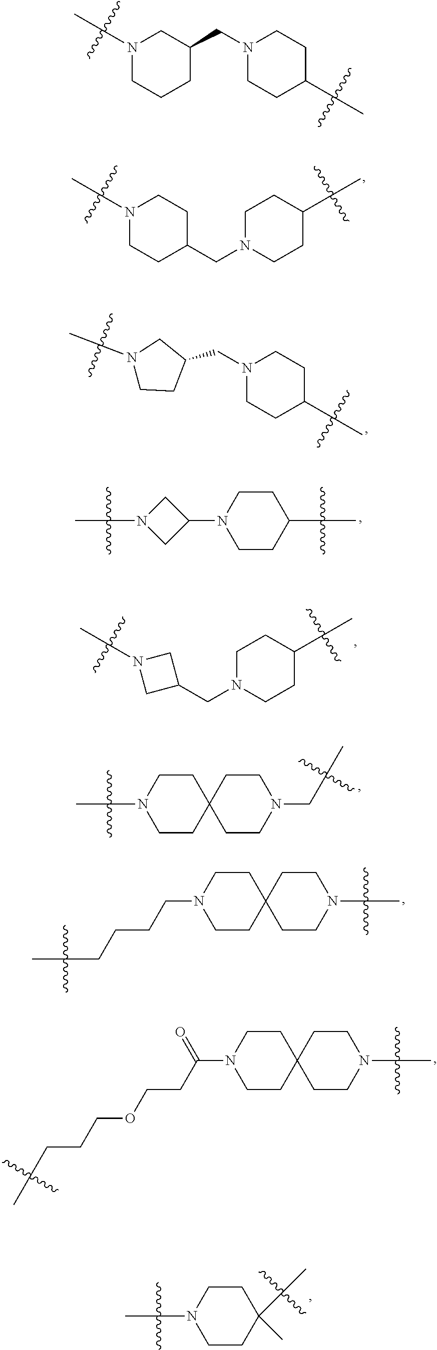

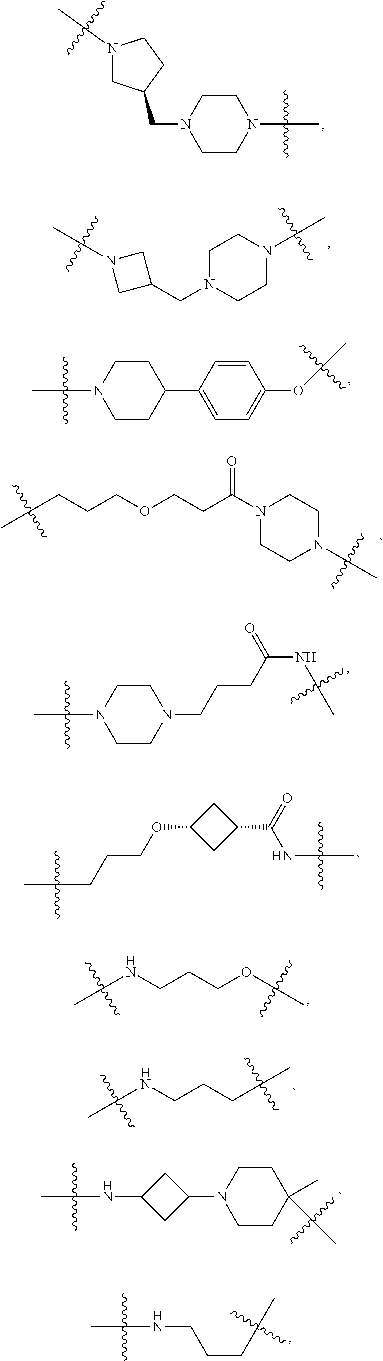

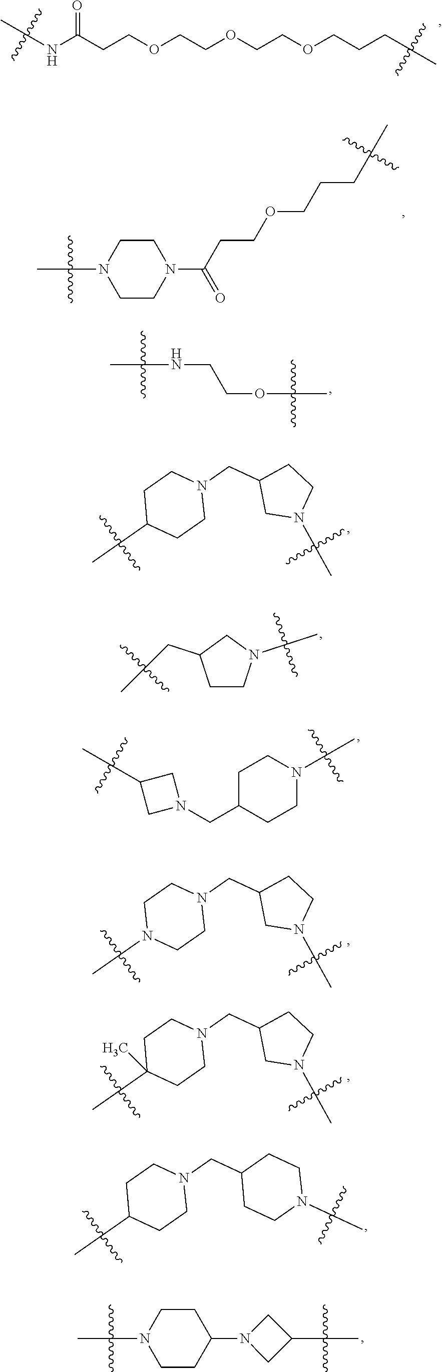

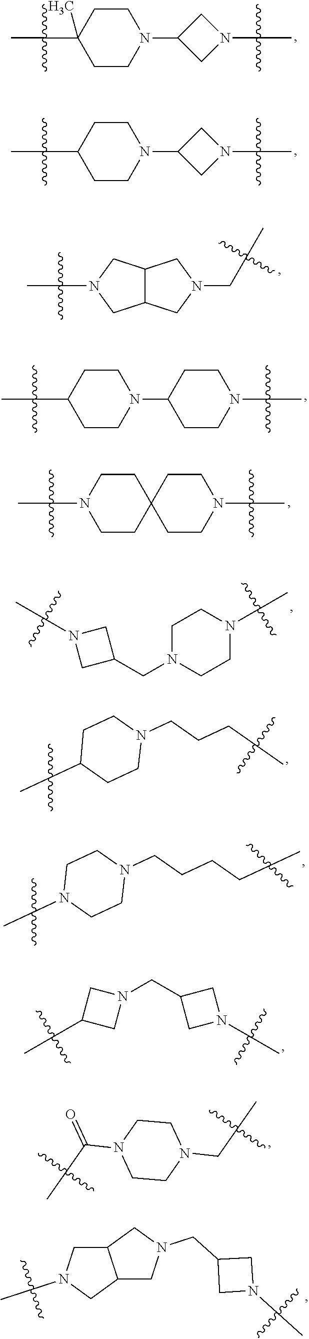

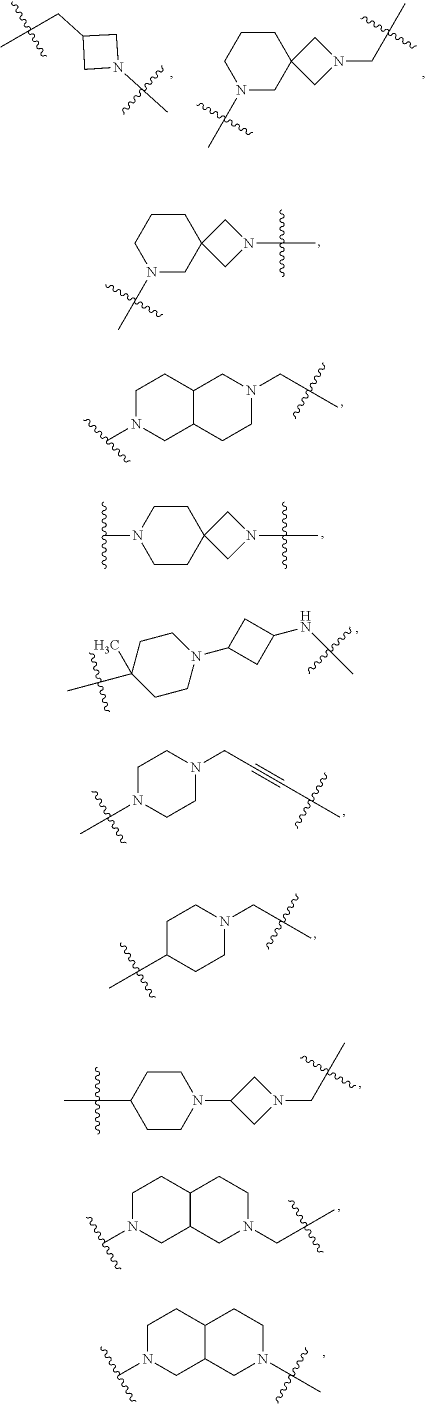

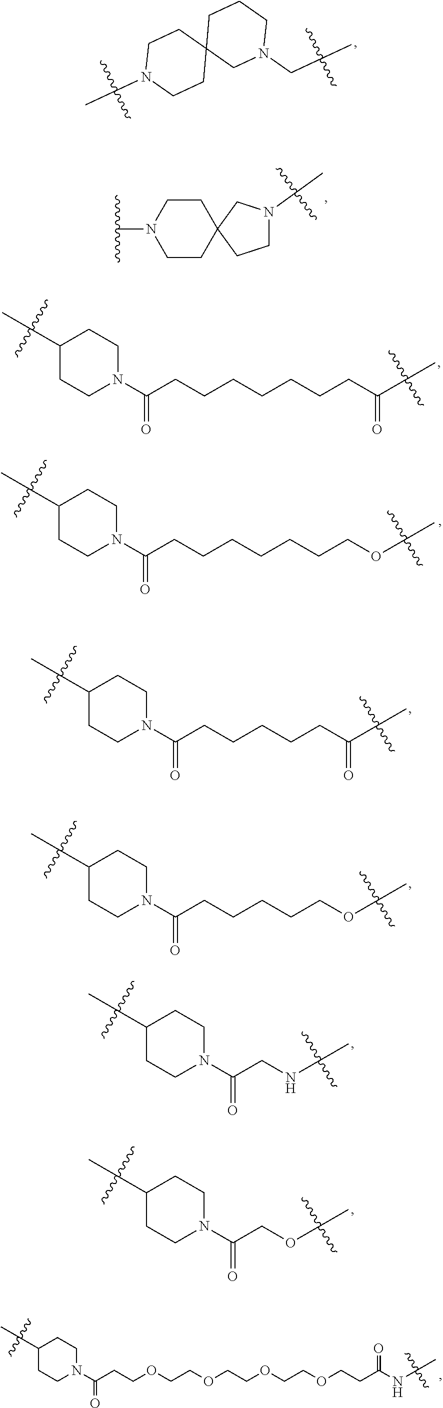

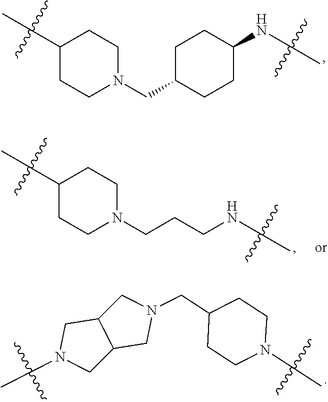 [SMILES: *N[C@H]1CC[C@H](CN2CCC(*)CC2)CC1 ; *NCCCN1CCC(*)CC1 ; *N1CCC(CN2CC3CN(*)CC3C2)CC1]

In some embodiments, Y is

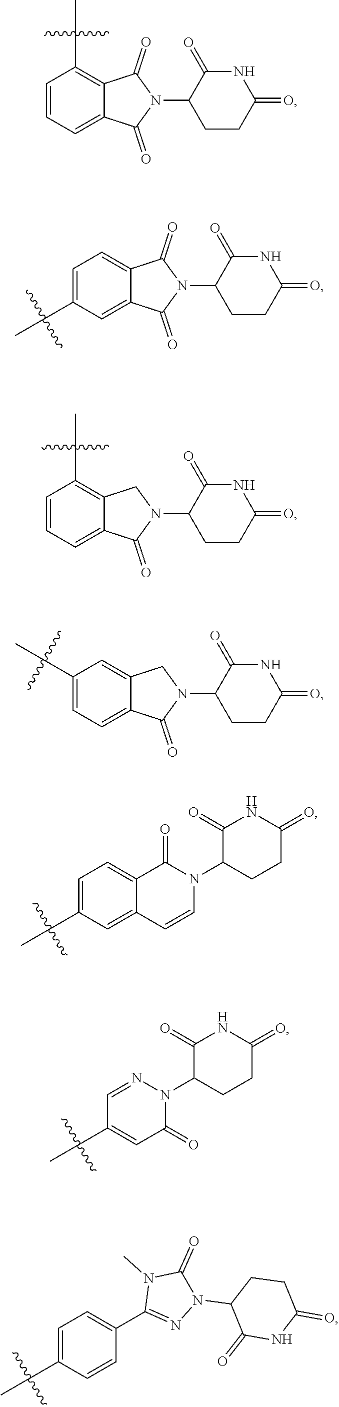

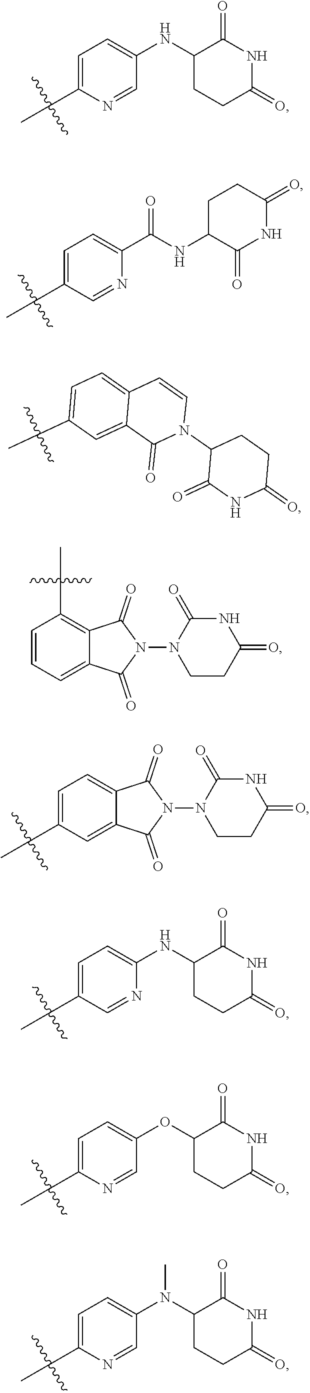

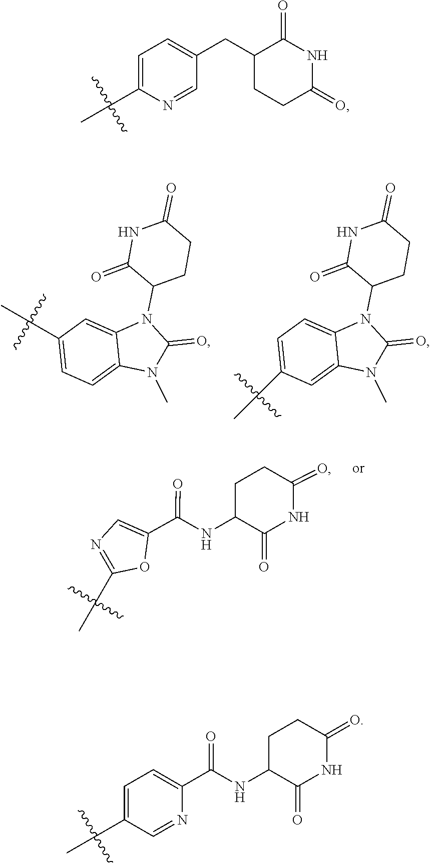 [SMILES: *c1ccc(CC2CCC(=O)NC2=O)cn1 ; *c1ccc2c(c1)n(C1CCC(=O)NC1=O)c(=O)n2C ; *c1ccc2c(c1)n(C)c(=O)n2C1CCC(=O)NC1=O ; *c1ncc(C(=O)NC2CCC(=O)NC2=O)o1 ; *c1ccc(C(=O)NC2CCC(=O)NC2=O)nc1]

In some embodiments, W is N.

In some embodiments, D is a bond.

The present invention also provides a compound of Formula (B)

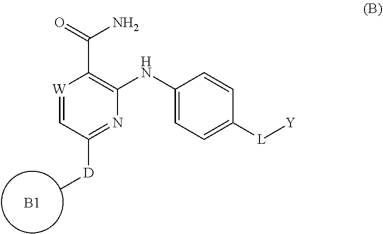

or a pharmaceutically acceptable salt thereof, wherein W is CH or N; D is a bond or —NH—; ring B1 is a 4-6 membered, fully saturated, partially unsaturated, or fully unsaturated monocyclic heterocycle or a 8-10 membered fully saturated spiro bicyclic heterocycle, wherein ring B1 has 1-3 heteroatoms independently selected from N, O, or S, and is optionally substituted with 1-3 groups selected from halo, —CH₃, —CF₃, —C(O)OH, —CH₂OH, or a 5 membered heterocycloalkyl optionally substituted with oxo and having 1-2 heteroatoms independently selected from N or O; L is —X¹—X²—X³—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro or fused bicyclic heterocycloalkyl having 1-3 heteroatoms independently selected from N, O, or S, or 4-6 membered monocyclic heterocycloalkyl having 1-2 heteroatoms independently selected from N, O, or S, wherein each of the monocyclic and bicyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having 1-2 heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having 1-2 heteroatoms independently selected from N, O, or S wherein the heterocycloalkyl is optionally substituted with —CH₃; each R is independently —H or —C₁₋₃ alkyl; each of m, n, and p is independently an integer from 1 to 3; and Y is

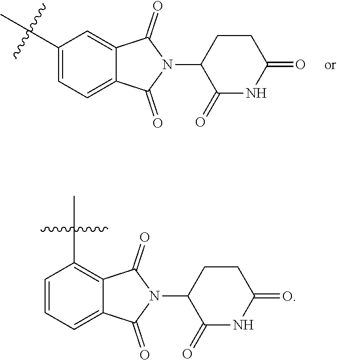

In some embodiments, ring B1 is

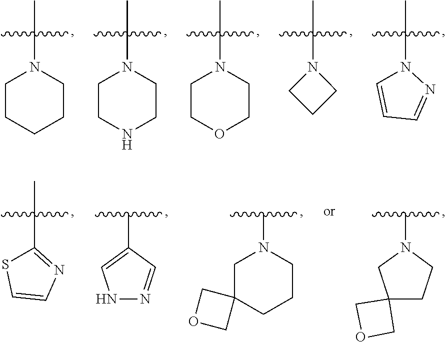

and ring B1 is optionally substituted 1-3 groups selected from —CH₃, —CH₂OH, —C(O)OH, —CF₃, —F,

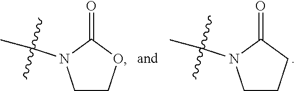 [SMILES: *N1CCOC1=O ; *N1CCCC1=O]

For example, ring B1 is

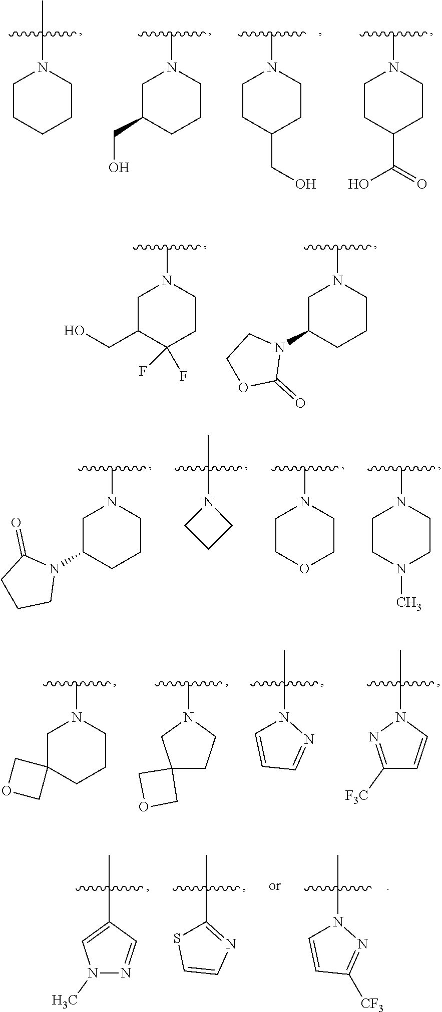

In other examples, ring B1 is

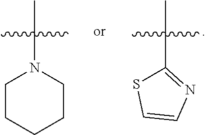 [SMILES: *N1CCCCC1 ; *c1nccs1]

In some embodiments, X¹ is

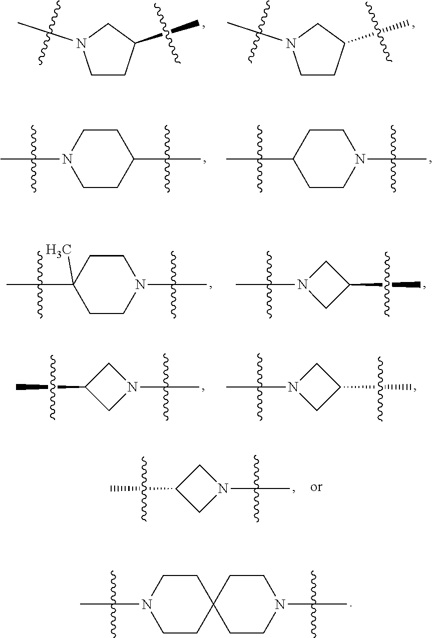

In some embodiments, X² is a bond, —C₁₋₅ alkyl-, 4-6 membered monocyclic cycloalkyl, or 4-6 membered monocyclic heterocycloalkyl having 1-2 heteroatoms independently selected from N, O, or S. For example, X² is a bond, —C₁₋₃ alkyl-, —C(O)—,

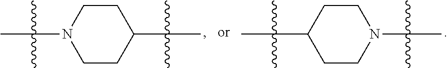 [SMILES: *C1CCN(*)CC1 ; *C1CCN(*)CC1]

In some embodiments, X³ is a bond, —C₁₋₄ alkyl-, —N(R)—, —(O—CH₂—CH₂)ₚ—, —(CH₂—CH₂—O)ₚ—, or a 4-6 membered heterocycloalkyl having 1-2 heteroatoms independently selected from N, O, or S wherein the heterocycloalkyl is optionally substituted with —CH₃. For example, X³ is a bond,

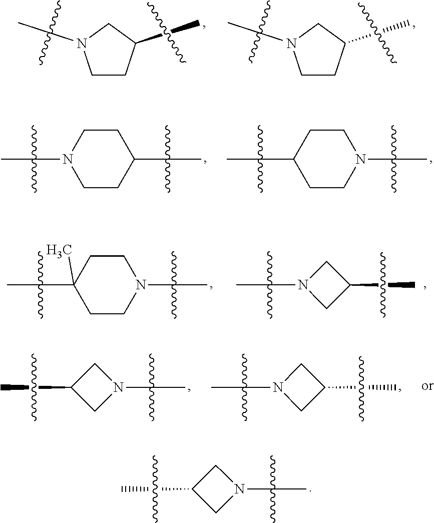

In some embodiments L is

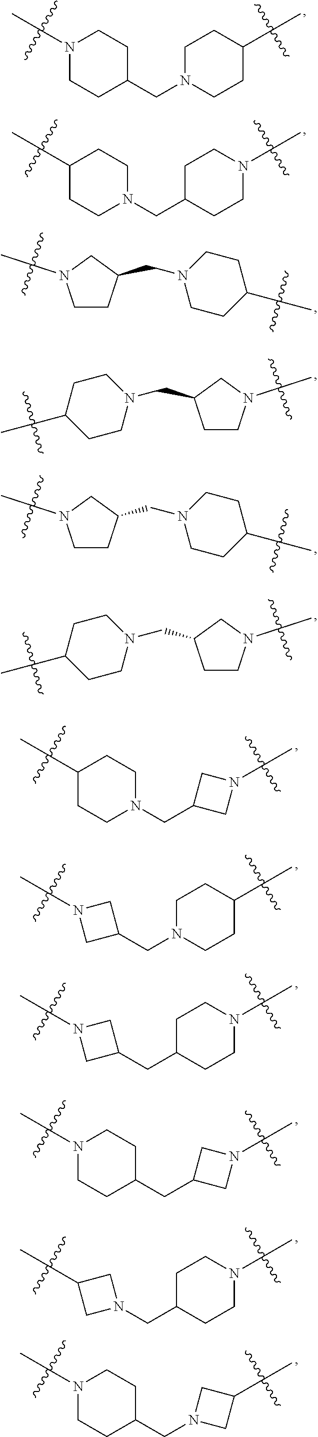

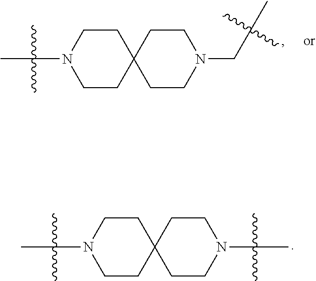 [SMILES: *CN1CCC2(CCN(*)CC2)CC1 ; *N1CCC2(CC1)CCN(*)CC2]

In some embodiments, W is N and D is a bond.

The present invention also provides a compound of Formula (C)

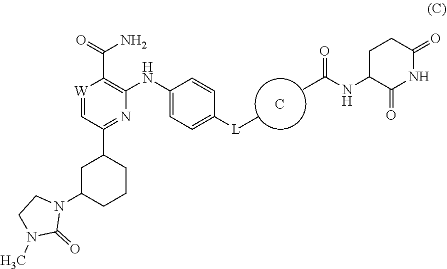

or a pharmaceutically acceptable salt thereof, wherein W is CH or N; ring C is phenyl or a saturated, partially unsaturated, or fully unsaturated 5-6 membered monocyclic heterocycle having 1-2 heteroatoms independently selected from N, O, or S, wherein each of the phenyl and heterocycle of ring C is optionally substituted; L is —X¹—X²—X³—; X¹ is —C(O)—N(R)—, —N(R)—C(O)—, —(O—CH₂—CH₂)ₘ—, —O—(C₆H₄)—, —(O—CH₂—CH₂—CH₂)ₘ—, —C₁₋₅ alkyl-, 7-12 membered spiro bicyclic heterocycloalkyl having 1-3 heteroatoms independently selected from N, O, or S, or 4-6 membered monocyclic heterocycloalkyl having 1-2 heteroatoms independently selected from N, O, or S, wherein each of the bicyclic heterocycloalkyl and the monocyclic heterocycloalkyl of X¹ is optionally substituted with —CH₃; X² is a bond, —(O—CH₂—CH₂)ₙ—, —(CH₂—CH₂—O)—, —N(R)—C(O)—, —N(R)—, —C(O)—, —C₁₋₅ alkyl-, 4-6 membered monocyclic cycloalkyl, or 4-6 membered monocyclic heterocycloalkyl having 1-2 heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having 1-2 heteroatoms independently selected from N, O, or S wherein the heterocycloalkyl is optionally substituted with —CH₃; each R is independently —H or —C₁₋₃ alkyl; and each of m, n, and p is independently an integer from 1 to 3.

In some embodiments, ring C is

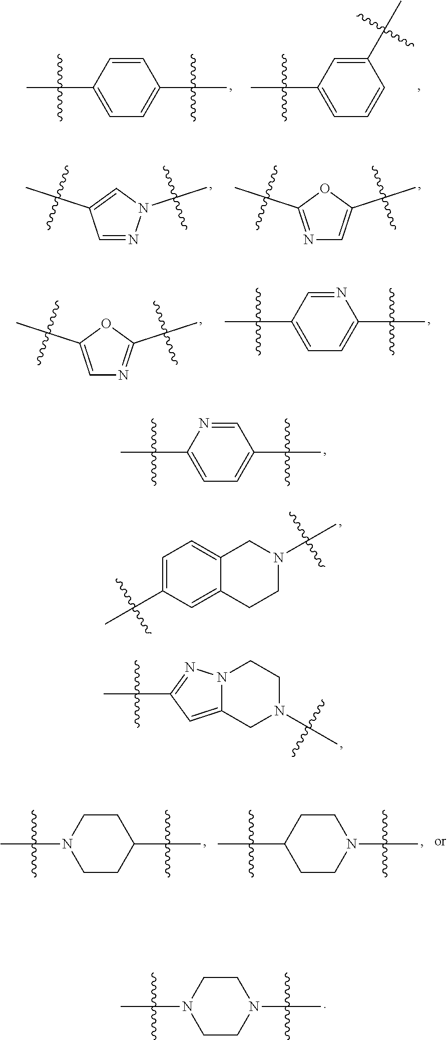

For example, ring C is

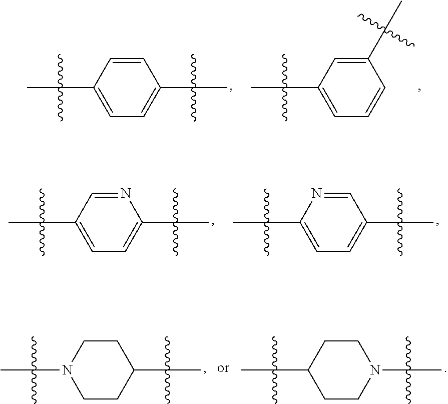 [SMILES: *c1ccc(*)cc1 ; *c1cccc(*)c1 ; *c1ccc(*)nc1 ; *c1ccc(*)nc1 ; *C1CCN(*)CC1 ; *C1CCN(*)CC1]

In some embodiments, X¹ is a 4-6 membered monocyclic heterocycloalkyl having 1-2 heteroatoms independently selected from N, O, or S. For example, X¹ is

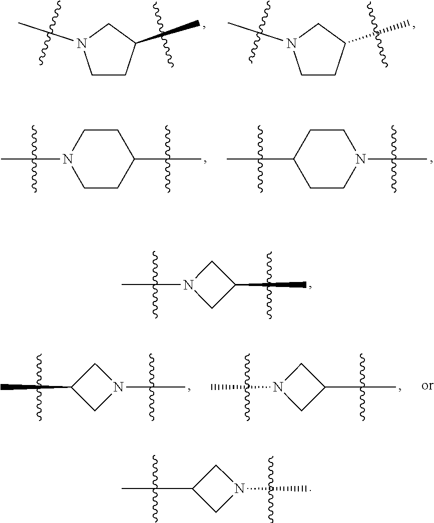

In some embodiments, X² is a bond, —C₁₋₅ alkyl-, 4-6 membered monocyclic cycloalkyl, or 4-6 membered monocyclic heterocycloalkyl having 1-2 heteroatoms independently selected from N, O, or S. For example, X² is a bond or —C₁₋₃ alkyl-.

In some embodiments, X³ is a 4-6 membered cycloalkyl, —N(R)—, or a 4-6 membered heterocycloalkyl having 1-2 heteroatoms independently selected from N, O, or S wherein the heterocycloalkyl is optionally substituted with —CH₃. For example, X³ is

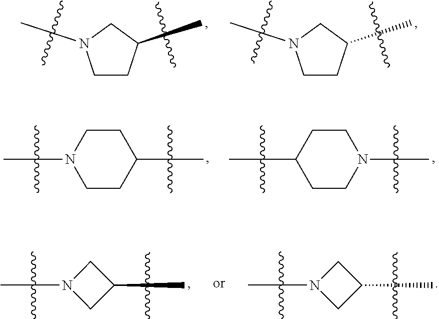

In some embodiments, L is

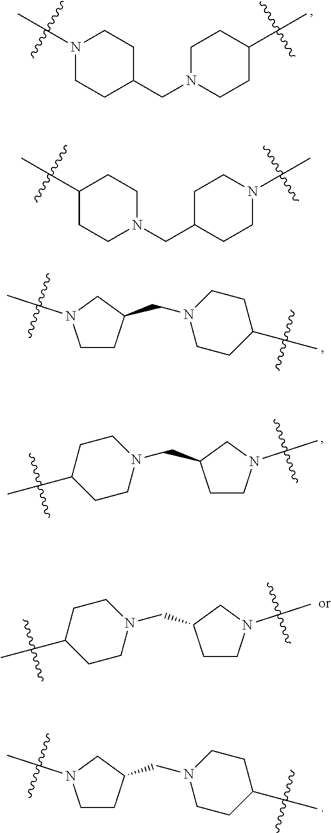 [SMILES: *C1CCN(CC2CCN(*)CC2)CC1 ; *C1CCN(CC2CCN(*)CC2)CC1 ; *C1CCN(C[C@H]2CCN(*)C2)CC1 ; *C1CCN(C[C@@H]2CCN(*)C2)CC1 ; *C1CCN(C[C@H]2CCN(*)C2)CC1 ; *C1CCN(C[C@@H]2CCN(*)C2)CC1]

The present invention also provides a compound of Formula (D)

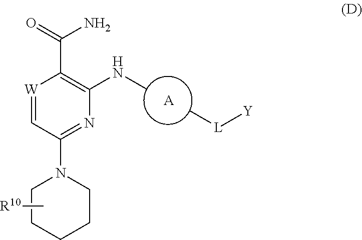

or a pharmaceutically acceptable salt thereof, wherein W is CH or N; ring A is

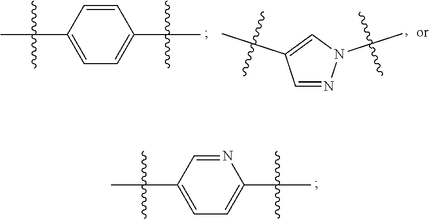 [SMILES: *c1ccc(*)cc1 ; *c1cnn(*)c1 ; *c1ccc(*)nc1]

L is —X¹—X²—X³—; X¹ is —C₁₋₅ alkyl- or 4-6 membered monocyclic heterocycloalkyl having 1-2 heteroatoms independently selected from N, O, or S wherein the monocyclic heterocycloalkyl of X¹ is optionally substituted with —CH₃; X² is a bond, —C₁₋₅ alkyl-, or 4-6 membered monocyclic heterocycloalkyl having 1-2 heteroatoms independently selected from N, O, or S wherein the monocyclic heterocycloalkyl of X¹ is optionally substituted with —CH₃; X³ is a bond, —C₁₋₄ alkyl-, 4-6 membered monocyclic cycloalkyl, or 4-6 membered heterocycloalkyl having 1-2 heteroatoms independently selected from N, O, or S wherein the heterocycloalkyl is optionally substituted with —CH₃; Y is

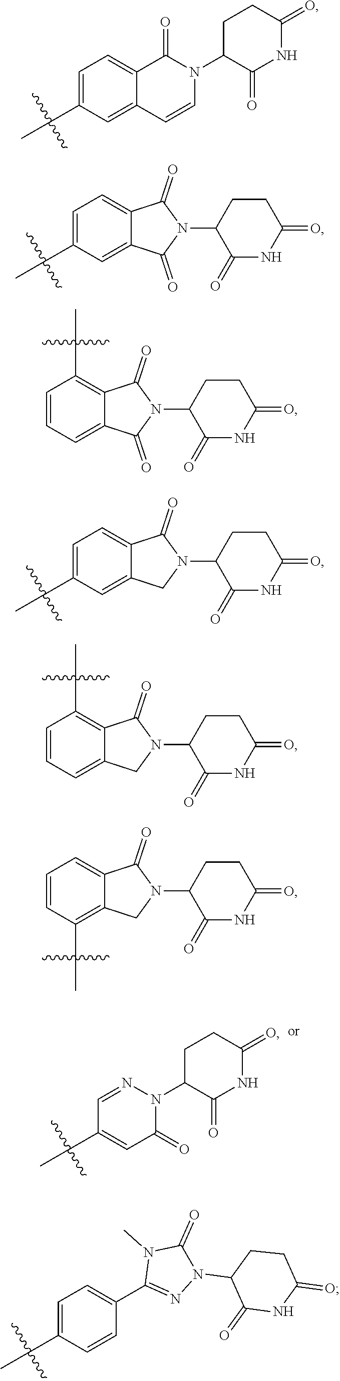

and R¹⁰ is halo, —H, —C₁₋₅ alkyl, -3-6 membered cycloalkyl, 5-6 membered heterocycloalkyl, —CN, —OH, —CF₃, —CH₂OH, —CH₂CH₂OH,

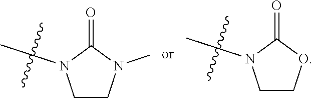

The present invention also provides a compound of Formula (D-1)

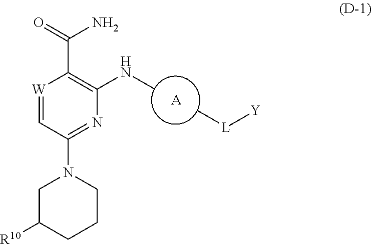

or a pharmaceutically acceptable salt thereof, wherein W is CH or N; ring A is

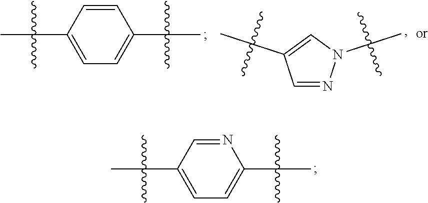 [SMILES: *c1ccc(*)cc1 ; *c1cnn(*)c1 ; *c1ccc(*)nc1]

L is —X¹—X²—X³—; X¹ is —C₁₋₅ alkyl- or 4-6 membered monocyclic heterocycloalkyl having 1-2 heteroatoms independently selected from N, O, or S wherein the monocyclic heterocycloalkyl of X¹ is optionally substituted with —CH₃; X² is a bond, —C₁₋₅ alkyl-, or 4-6 membered monocyclic heterocycloalkyl having 1-2 heteroatoms independently selected from N, O, or S wherein the monocyclic heterocycloalkyl of X¹ is optionally substituted with —CH₃; X³ is a bond, —C₁₋₄ alkyl-, 4-6 membered monocyclic cycloalkyl, or 4-6 membered heterocycloalkyl having 1-2 heteroatoms independently selected from N, O, or S wherein the heterocycloalkyl is optionally substituted with —CH₃; Y is

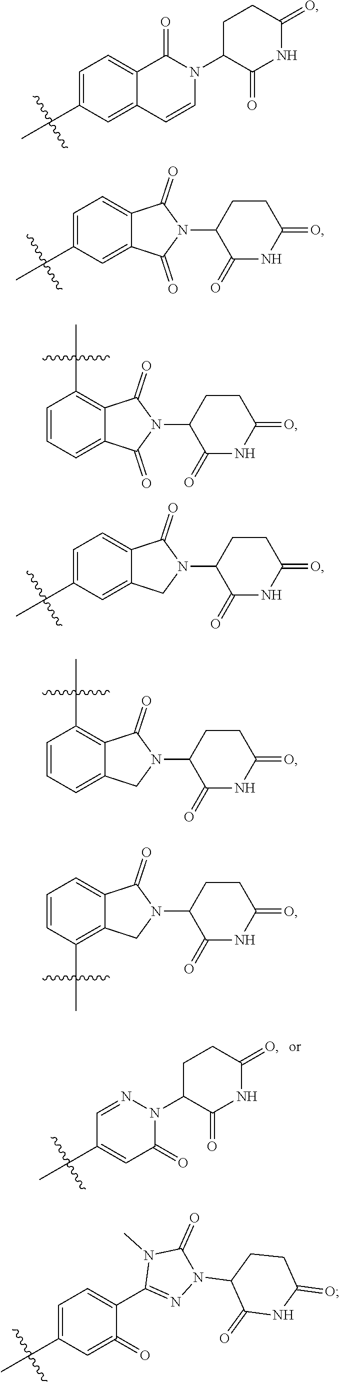

### and R¹⁰ is

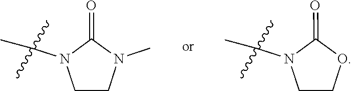

In some embodiments, the compound of Formula (D) is a compound of Formula (D-2):

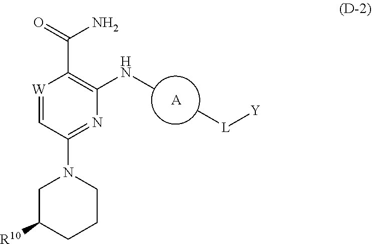

or a pharmaceutically acceptable salt thereof, wherein the terms ring A, L, Y, and R¹⁰ are as defined in the compound of Formula (A), (B), (C), (D), and (D-1).

In some embodiments, ring A is

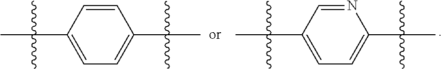 [SMILES: *c1ccc(*)cc1 ; *c1ccc(*)nc1]

In some embodiments, X¹ is a 4-6 membered monocyclic heterocycloalkyl having 1-2 heteroatoms independently selected from N, O, or S wherein the monocyclic heterocycloalkyl of X¹ is optionally substituted with —CH₃. For example, X¹ is

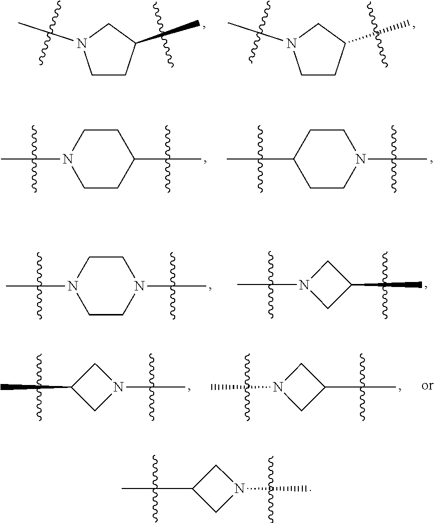

In some embodiments, X² is a bond, —C₁₋₅ alkyl-, 4-6 membered monocyclic cycloalkyl, or 4-6 membered monocyclic heterocycloalkyl having 1-2 heteroatoms independently selected from N, O, or S. For example, X² is a bond or —C₁₋₄ alkyl-.

In some embodiments, X³ is a bond, a 4-6 membered monocyclic cycloalkyl, or 4-6 membered monocyclic heterocycloalkyl having 1-2 heteroatoms independently selected from N, O, or S. For example, X³ is

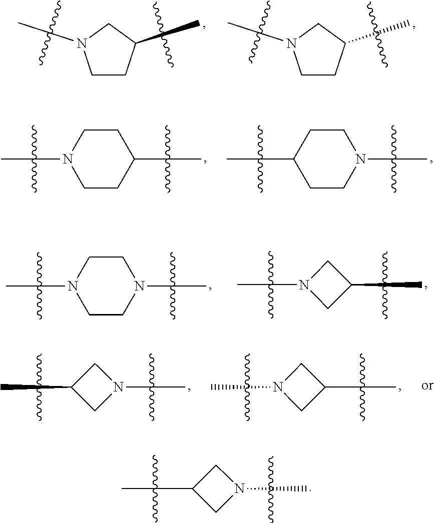

In some embodiments, L is

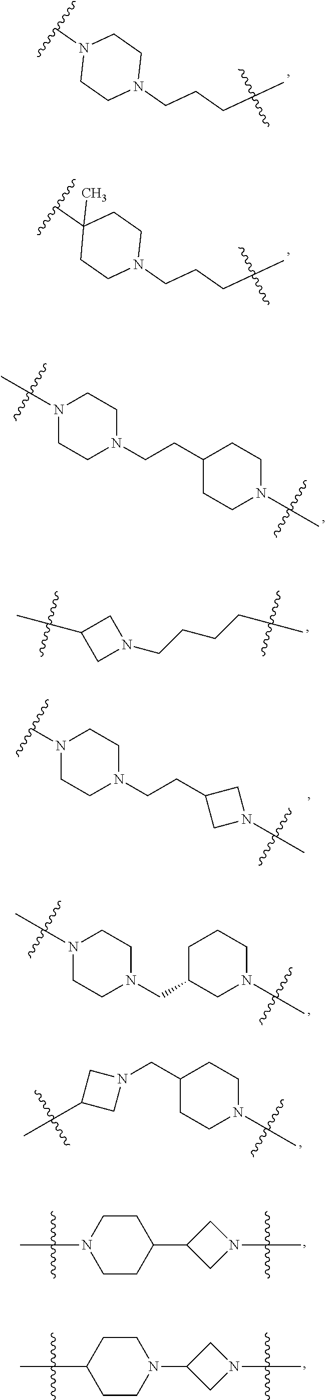

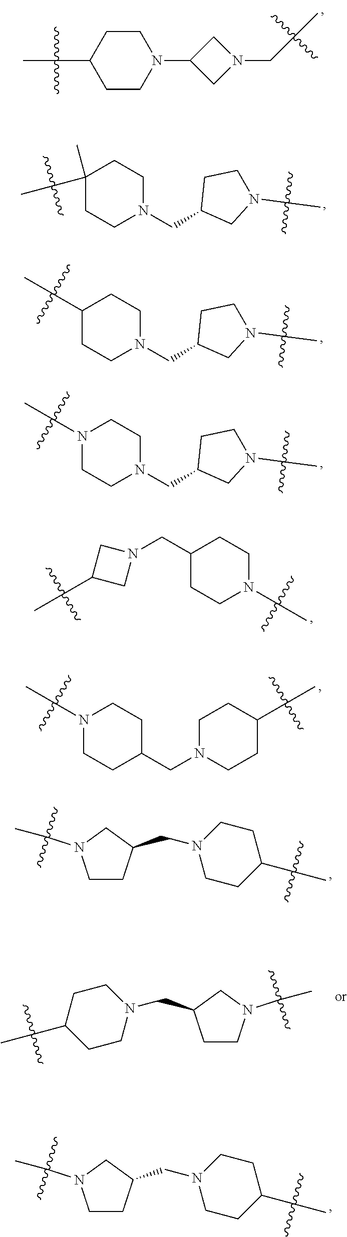

In some embodiments, R¹⁰ is

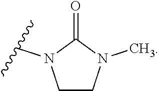 [SMILES: *N1CCN(C)C1=O]

In some embodiments, R¹⁰ is

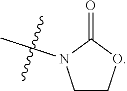 [SMILES: *N1CCOC1=O]

The present invention also provides a compound of Formula (E)

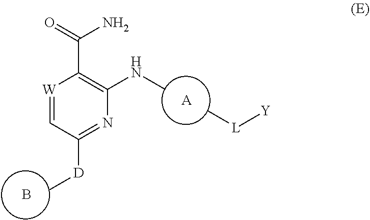

or a pharmaceutically acceptable salt thereof, wherein D is a bond or —NH—; W is N or CH; ring A is phenyl, a 9-10 membered bicyclic aryl, a 5-6 membered partially or fully unsaturated monocyclic heterocycle, or a 9-10 membered bicyclic heteroaryl, wherein the monocyclic heterocycle and bicyclic heteroaryl of ring A each possess 1-3 heteroatoms independently selected from N, O, or S; ring B is an optionally substituted 5-6 membered saturated, partially unsaturated, or fully unsaturated monocyclic heterocycle, or an optionally substituted 8-10 membered (e.g., 8-9 membered or 9-10 membered) spiro bicyclic heterocycle, wherein ring B has 1-3 heteroatoms independently selected from N, O, or S; L is —X¹—X²—X³—X⁴—X⁵—; X¹ is a bond, —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having 1-3 heteroatoms independently selected from N, O, or S, or 4-6 membered monocyclic heterocycloalkyl having 1-2 heteroatoms independently selected from N, O, or S, wherein each of the monocyclic and bicyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having 1-2 heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having 1-2 heteroatoms independently selected from N, O, or S wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, a 5-6 membered saturated, partially unsaturated, or fully unsaturated carbocycle, or a 5-6 membered saturated, partially unsaturated, or fully unsaturated heterocycle having 1-3 heteroatoms independently selected from N, O, or S; X⁵ is a bond, —N(R)—, or —C(O)—N(R)—; each R is independently —H or —C₁₋₃ alkyl; each of m, n, and p is independently an integer from 1 to 3 (e.g., 1, 2, or 3); and Y is

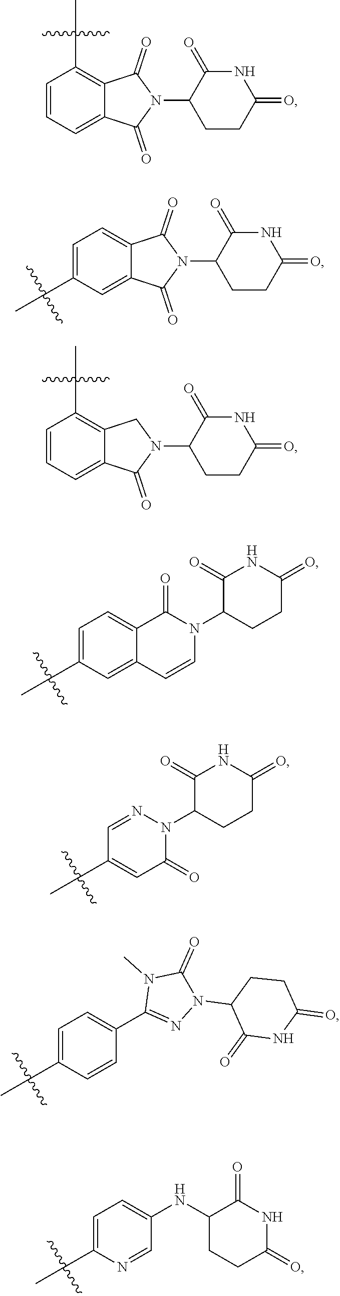

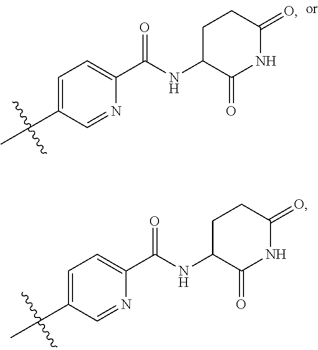 [SMILES: *c1ccc(C(=O)NC2CCC(=O)NC2=O)nc1 ; *c1ccc(C(=O)NC2CCC(=O)NC2=O)nc1]

wherein at least one of X¹, X², X³, X⁴, and X⁵ has a nitrogen atom, and Y is directly bonded to L at a nitrogen atom of X¹, X², X³, X⁴, or X⁵.

In some embodiments, ring B is

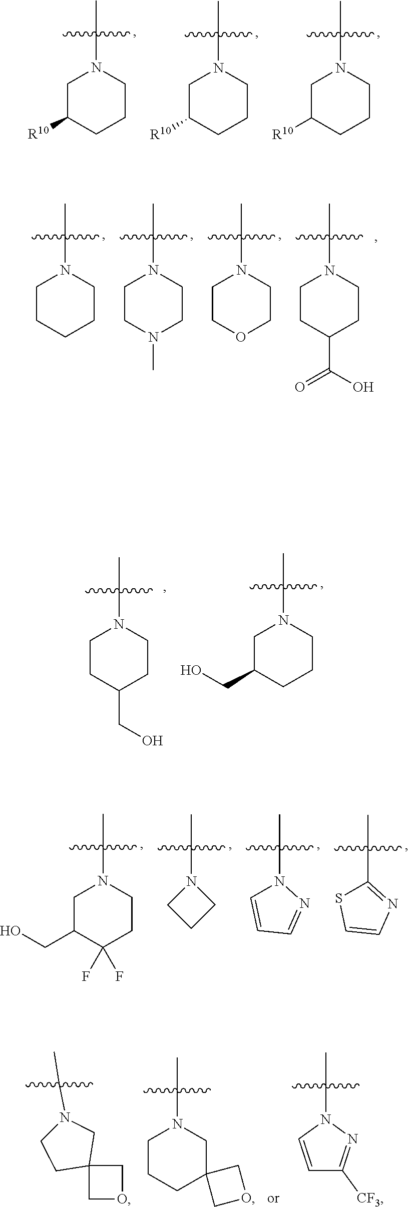

wherein R¹⁰ is

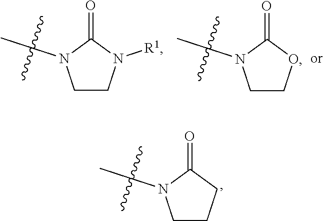

and wherein R¹ is a C₁₋₄ alkyl group. For example, ring B is

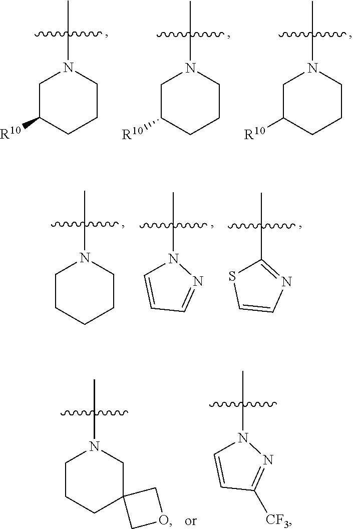

wherein R¹⁰ is

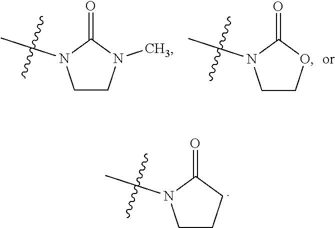 [SMILES: *N1CCN(C)C1=O ; *N1CCOC1=O ; *N1CCCC1=O]

In other examples, ring B is

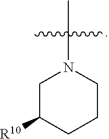

In some embodiments, R¹⁰ is

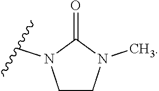 [SMILES: *N1CCN(C)C1=O]

In some embodiments, ring A is

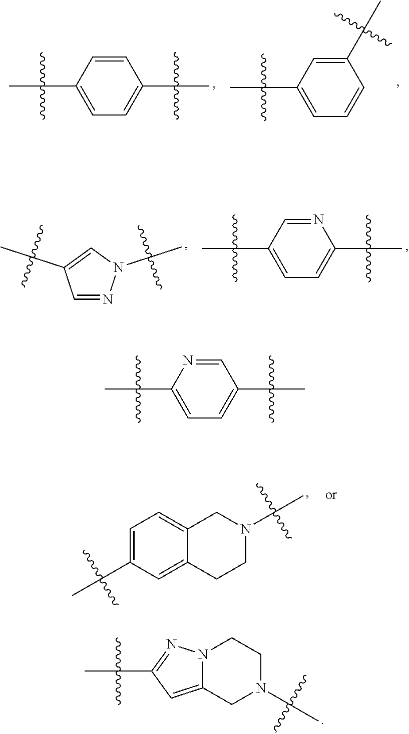

In some embodiments, X⁵ is —N(R)—.

In some embodiments, X⁵ is —C(O)—N(R)—.

In some embodiments, X⁵ is a bond.

In some embodiments, L is

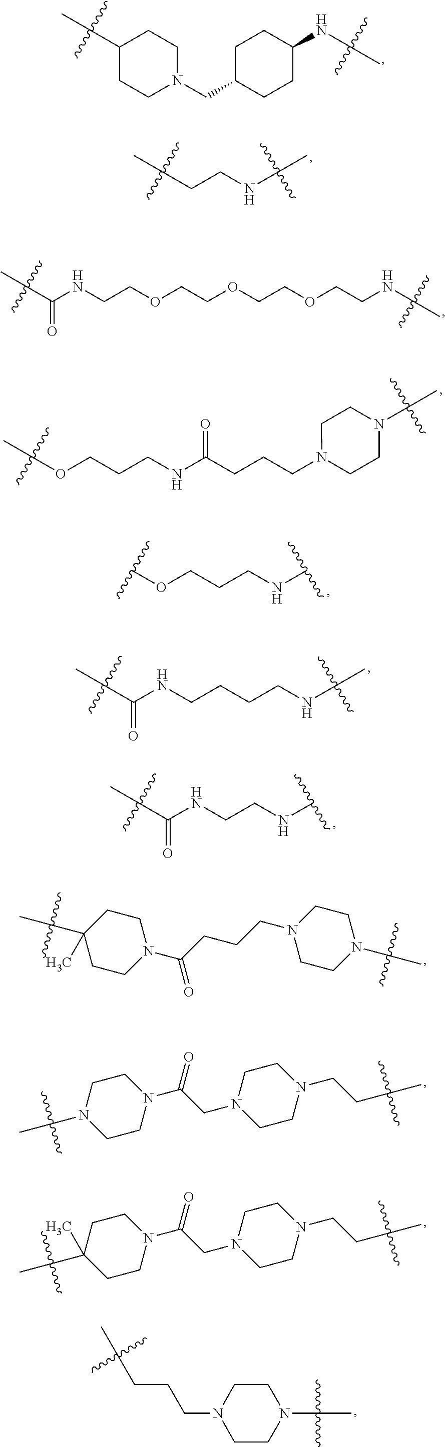

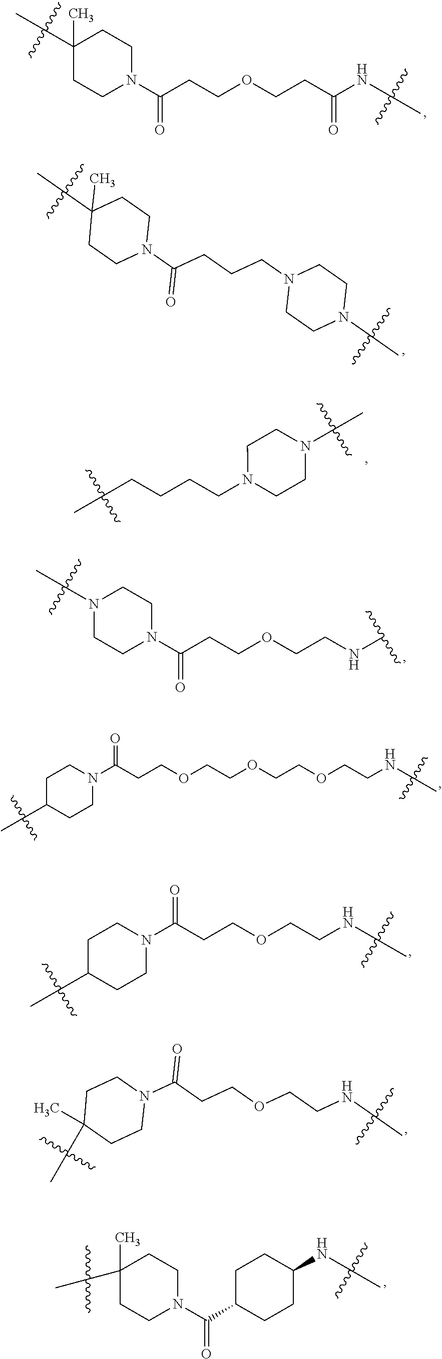

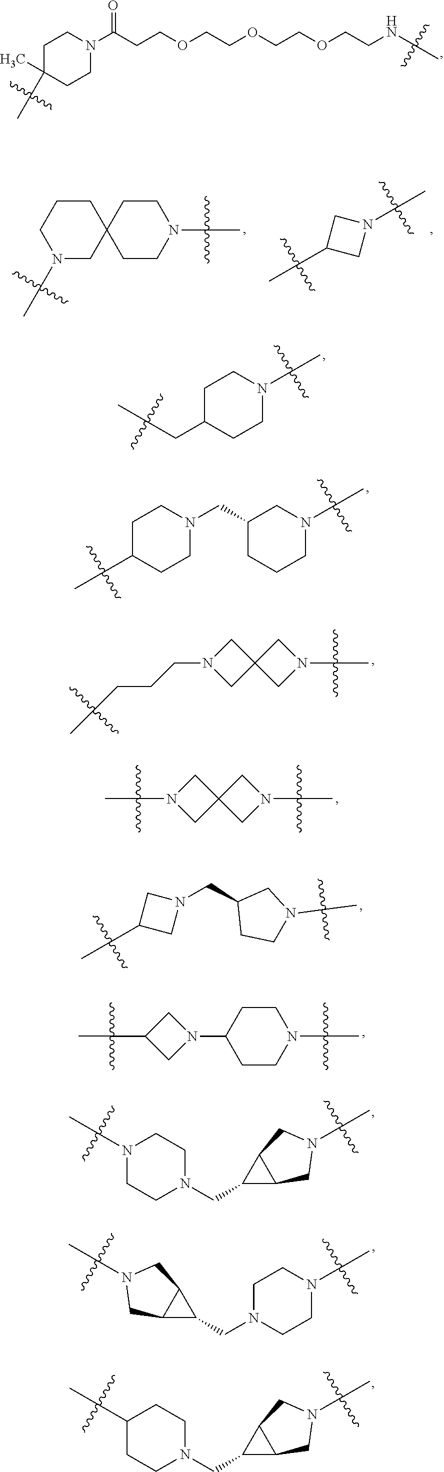

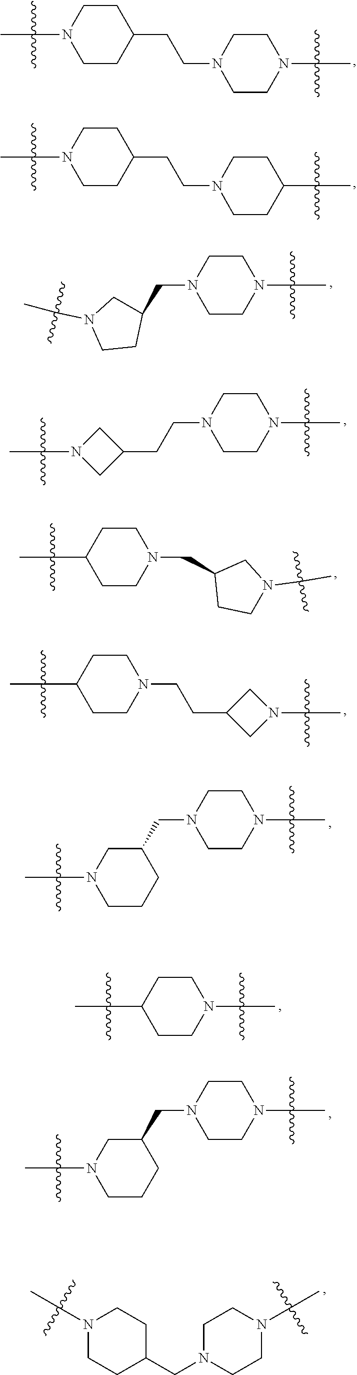

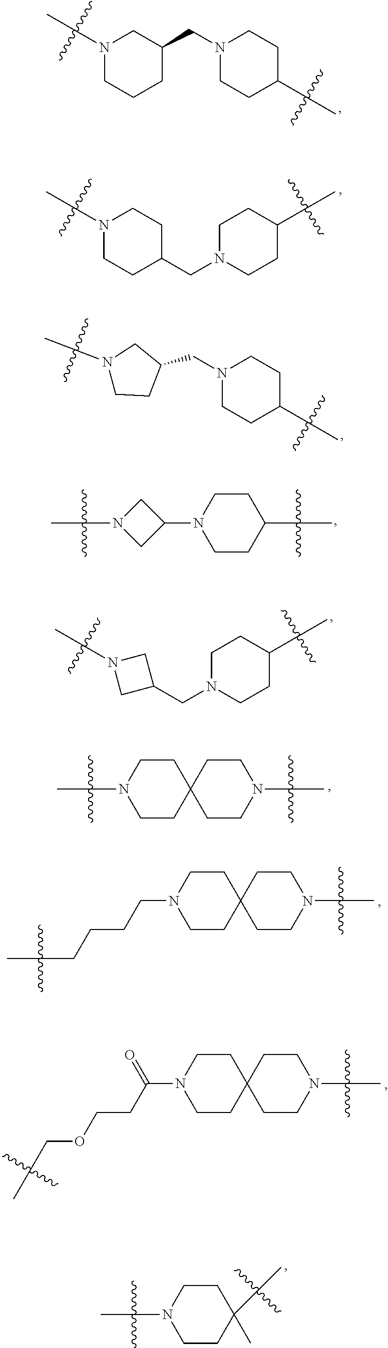

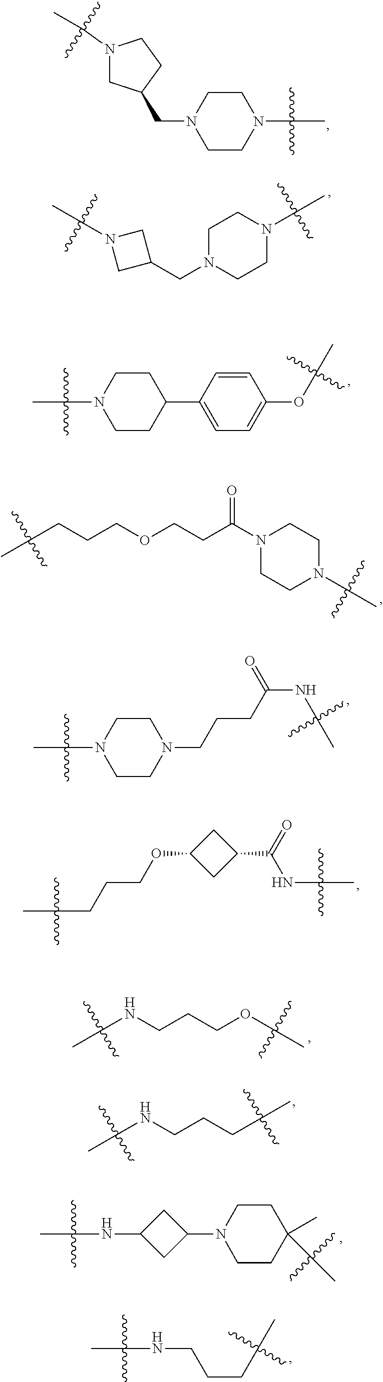

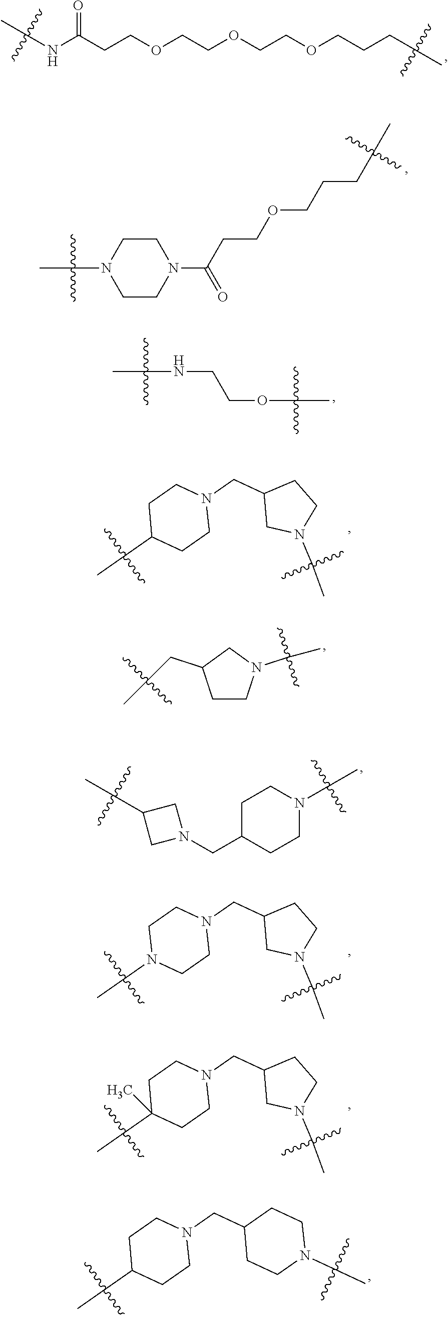

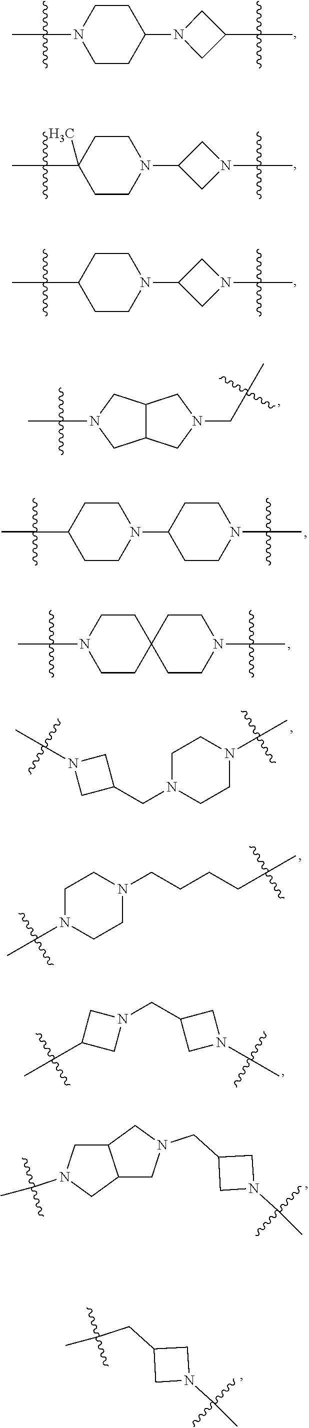

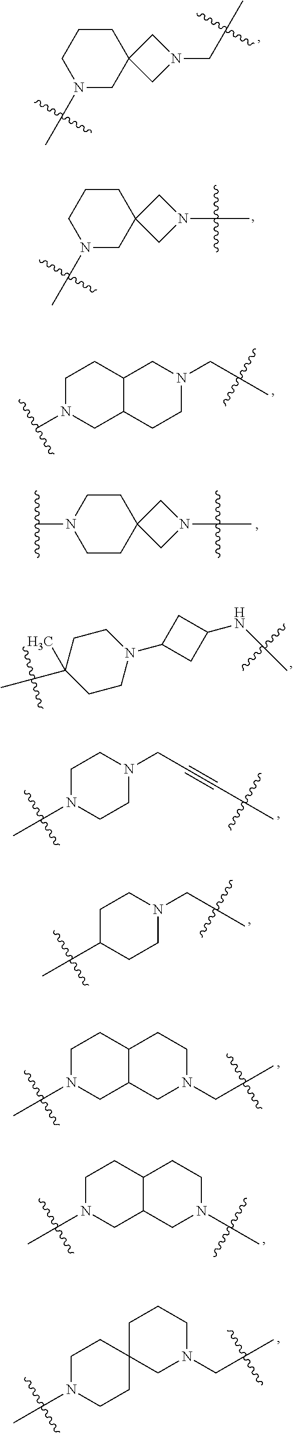

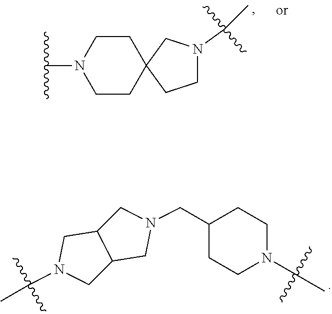 [SMILES: *N1CCC2(CC1)CCN(*)C2 ; *N1CCC(CN2CC3CN(*)CC3C2)CC1]

In some embodiments, Y is

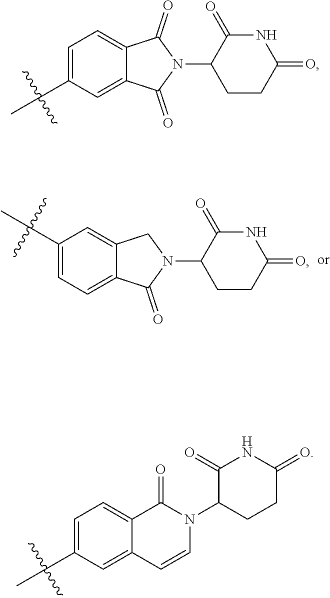 [SMILES: *c1ccc2c(c1)C(=O)N(C1CCC(=O)NC1=O)C2=O ; *c1ccc2c(c1)CN(C1CCC(=O)NC1=O)C2=O ; *c1ccc2c(=O)n(C3CCC(=O)NC3=O)ccc2c1]

The present invention also provides a compound of Formula (F)

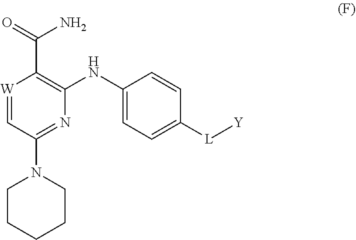

or a pharmaceutically acceptable salt thereof, wherein W is CH or N; L is —X¹—X²—X³—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having 1-3 heteroatoms independently selected from N, O, or S, or 4-6 membered monocyclic heterocycloalkyl having 1-2 heteroatoms independently selected from N, O, or S, wherein each of the monocyclic and bicyclic heterocycloalkyl of X¹ is optionally substituted with —CH₃; X² is a bond, —C₁₋₅ alkyl-, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having 1-2 heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having 1-2 heteroatoms independently selected from N, O, or S wherein the heterocycloalkyl is optionally substituted with —CH₃; each R is independently —H or —C₁₋₃ alkyl; each of m, n, and p is independently an integer from 1 to 3; and Y is

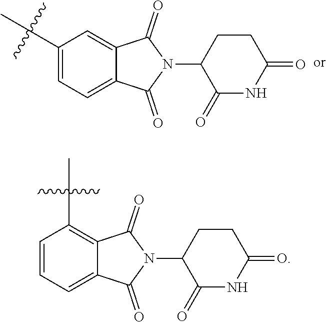 [SMILES: *c1ccc2c(c1)C(=O)N(C1CCC(=O)NC1=O)C2=O ; *c1cccc2c1C(=O)N(C1CCC(=O)NC1=O)C2=O]

In some embodiments, W is N.

In some embodiments, Y is

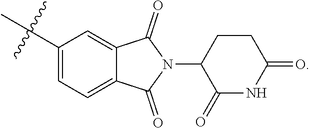 [SMILES: *c1ccc2c(c1)C(=O)N(C1CCC(=O)NC1=O)C2=O]

In some embodiments, X¹ is a 4-6 membered monocyclic heterocycloalkyl having 1-2 heteroatoms independently selected from N, O, or S, wherein each of the monocyclic heterocycloalkyl of X¹ is optionally substituted with —CH₃. For example, X¹ is

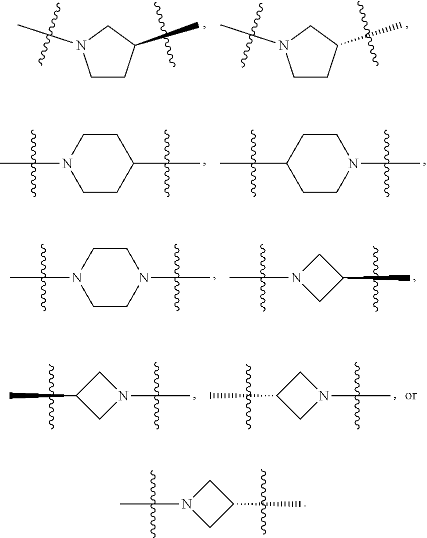

In some instances X¹ is

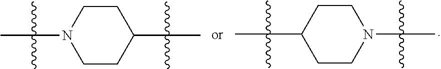 [SMILES: *C1CCN(*)CC1 ; *C1CCN(*)CC1]

In some embodiments, X² is a bond or —C₁₋₅ alkyl-.

In some embodiments, X³ is a 4-6 membered monocyclic heterocycloalkyl having 1-2 heteroatoms independently selected from N, O, or S. For example, X³ is

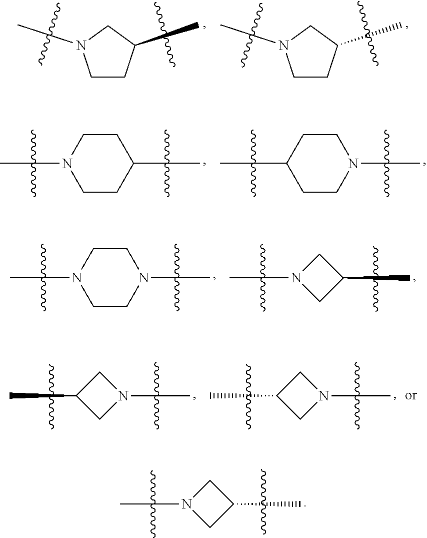

In some instances, X³ is

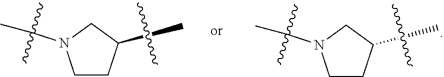

In some embodiments, L is

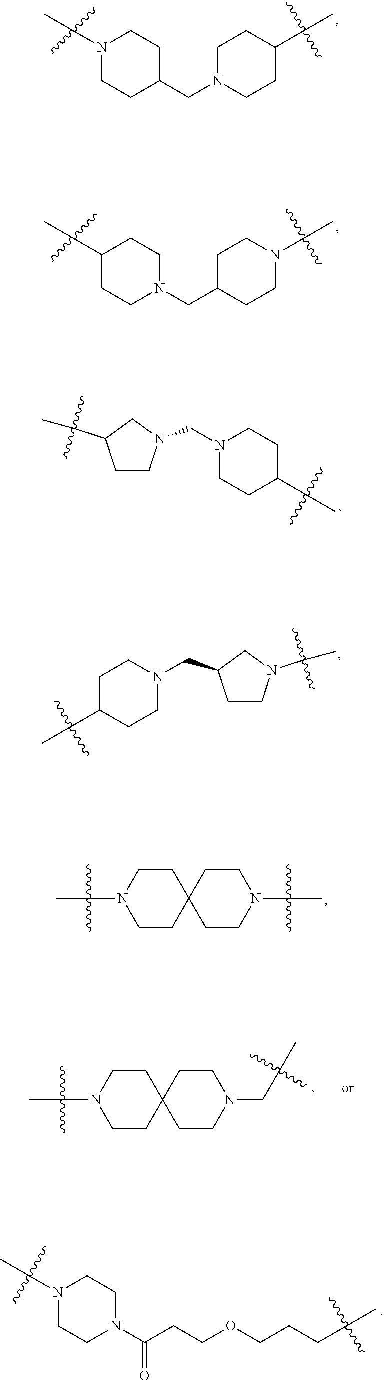

The present invention also provides a compound of Formula (G)

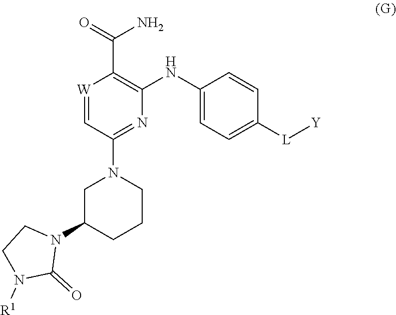

or a pharmaceutically acceptable salt thereof, wherein R¹, L, and Y are as defined for compounds of Formula (A), (B), (C), (D), (E), (F), (X), and (I).

In some embodiments, R¹ is methyl.

In some embodiments, Y is

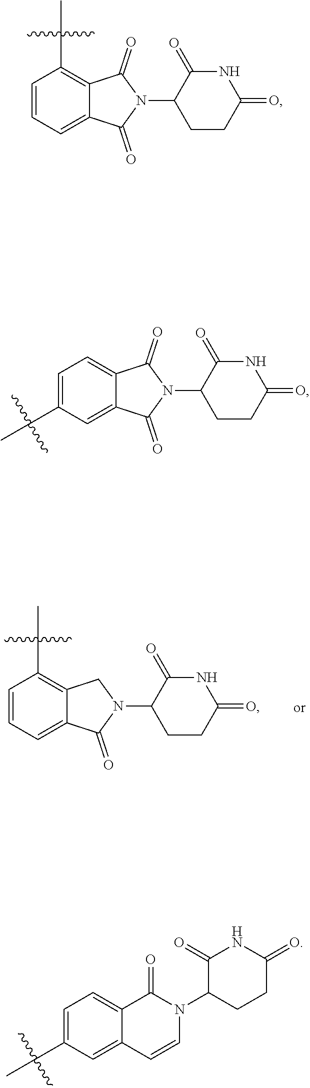 [SMILES: *c1cccc2c1C(=O)N(C1CCC(=O)NC1=O)C2=O ; *c1ccc2c(c1)C(=O)N(C1CCC(=O)NC1=O)C2=O ; *c1cccc2c1CN(C1CCC(=O)NC1=O)C2=O ; *c1ccc2c(=O)n(C3CCC(=O)NC3=O)ccc2c1]

In some embodiments, W is N.

The present invention also provides a compound of Formula (H)

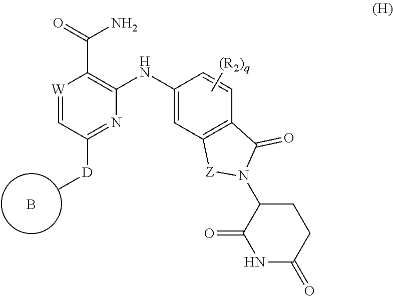

or a pharmaceutically acceptable salt thereof, wherein ring B, R², Z, W, D, and q are as defined in the compound of Formula (A), (B), (C), (D), (E), (F), (G), (X), and (I).

In some embodiments, q is 0.

The present invention also provides a compound of Formula (J)

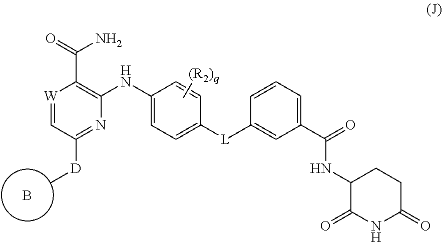

or a pharmaceutically acceptable salt thereof, wherein ring B, D, W, R², q, and L are as defined in the compound of Formula (A), (B), (C), (D), (E), (F), (H), (X), and (I).

The present invention also provides a compound of Formula (K)

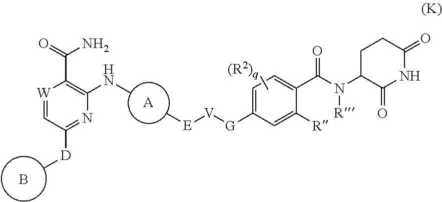

or a pharmaceutically acceptable salt thereof, wherein ring A is

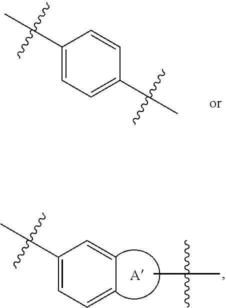

wherein ring A is optionally and independently substituted with up to 3 substituents selected from halo, CN, carboxyl, NH₂, and optionally substituted C₁₋₆ alkyl; V is a bond or —CH₂—; and E and G are each independently a 5-6 membered heterocycloalkyl, wherein each heterocycloalkyl contains at least one nitrogen atom. Ring B, W, R², q, R″, R′″, and ring A′ are as defined in the compound of Formula (A).

In some embodiments, D is a bond and W is a nitrogen atom.

The present invention also provides a compound of Formula (M)

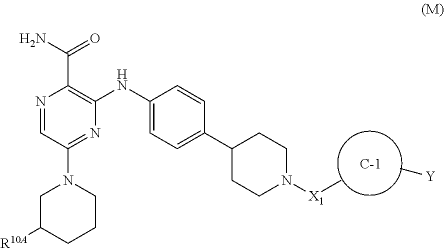

or a pharmaceutically acceptable salt thereof, wherein R^{10A} is —H,

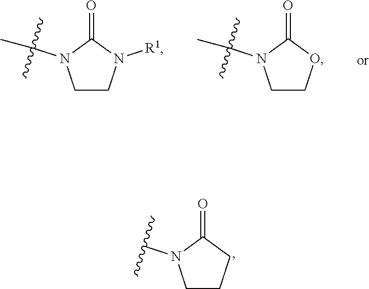

wherein R¹ is C₁₋₄ alkyl; X¹ is —C₁₋₅ alkyl-; ring C-1 is a 5-6 membered heterocycloalkyl having 1 nitrogen atom; and Y is

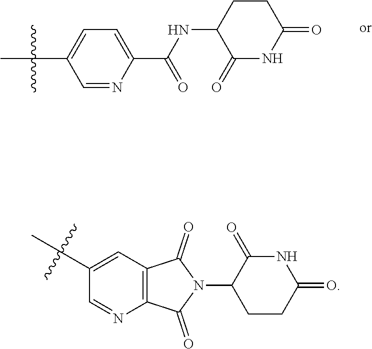 [SMILES: *c1ccc(C(=O)NC2CCC(=O)NC2=O)nc1 ; *c1cnc2c(c1)C(=O)N(C1CCC(=O)NC1=O)C2=O]

In some embodiments, R^{10A} is —H or

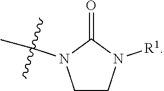

In some embodiments, R^{10A} is

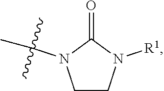

and R¹ is methyl, ethyl, propyl, iso-propyl, butyl, sec-butyl, or iso-butyl. For example, R¹ is methyl.

In some embodiments, X¹ is methylene, ethylene, or propylene. For instance, X¹ is methylene.

In some embodiments, ring C-1 is

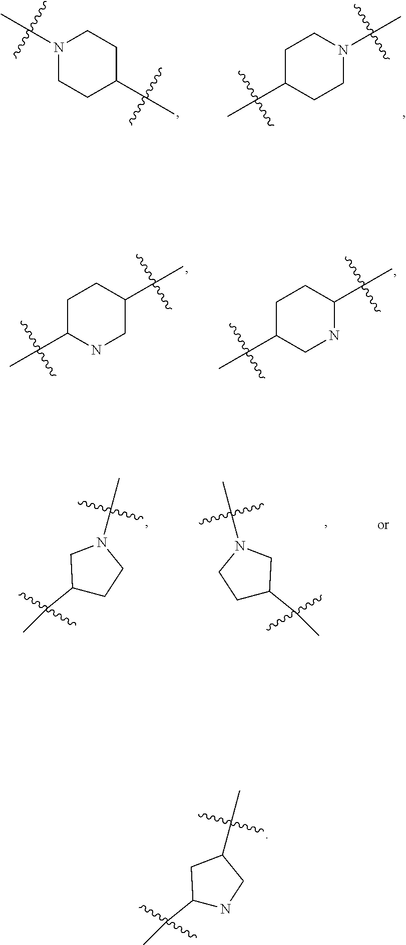

For instance, ring C-1 is

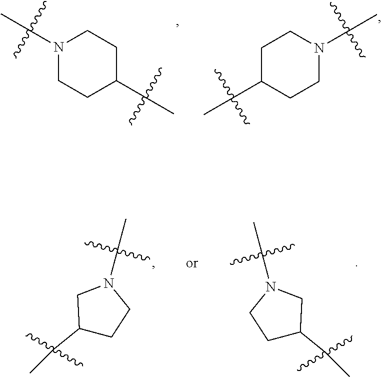 [SMILES: *C1CCN(*)CC1 ; *C1CCN(*)CC1 ; *C1CCN(*)C1 ; *C1CCN(*)C1]

The present invention also provides a compound of Formula (X)

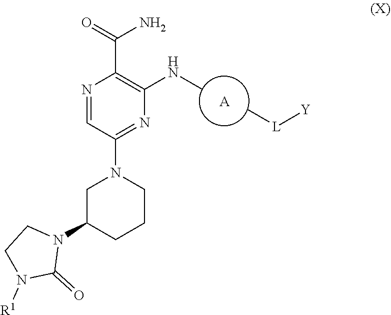

or a pharmaceutically acceptable salt thereof, wherein R¹ is C₁₋₃ alkyl; ring A is phenyl, 5-6 membered partially or fully unsaturated monocyclic heterocycle, 9-10 membered bicyclic aryl, or 9-10 membered bicyclic heteroaryl, wherein the heterocycle and the bicyclic heteroaryl of ring A each independently have 1-3 heteroatoms independently selected from N, O, or S; L is —X¹—X²—X³—X⁴—X⁵—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having 1-3 heteroatoms independently selected from N, O, or S wherein the bicyclic heterocycloalkyl of X¹ is optionally substituted with —CH₃, or 4-6 membered monocyclic heterocycloalkyl having 1-2 heteroatoms independently selected from N, O, or S wherein the monocyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having 1-2 heteroatoms independently selected from N, O, or S; X³ is a bond, —C₁₋₄ alkyl-, —C≡C-4-6 membered cycloalkyl, —N(R)—, —(O—CH₂—CH₂)ₚ—, —(CH₂—CH₂—O)ₚ—, 4-6 membered heterocycloalkyl having 1-2 heteroatoms independently selected from N, O, or S wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, a 5-6 membered saturated, partially unsaturated, or fully unsaturated carbocycle, or a 5-6 membered saturated, partially unsaturated, or fully unsaturated heterocycle having 1-3 heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from 1 to 3; Y is

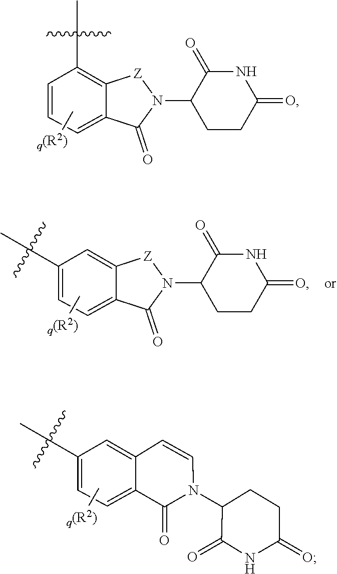

wherein each R² is independently halo or C₁₋₄ alkyl; each Z is —C(R^{A})₂— or —C(O)—; each R^{A} is independently —H or C₁₋₄ alkyl; and q is 0, 1, or 2.

In some embodiments, q is 0. In other embodiments, q is 1 and R² is —F.

In some embodiments, Z is —CH— or —C(O)—.

In some embodiments, Y is

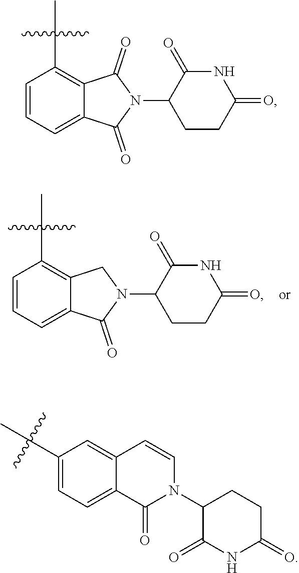

In some embodiments, Y is

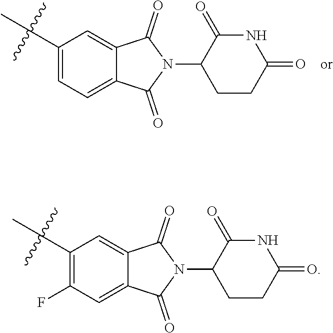

In some embodiments, R¹ is methyl, ethyl, or propyl. For example, R¹ is methyl.

In some embodiments, each R is independently —H or —CH₃.

In some embodiments, ring A is selected from

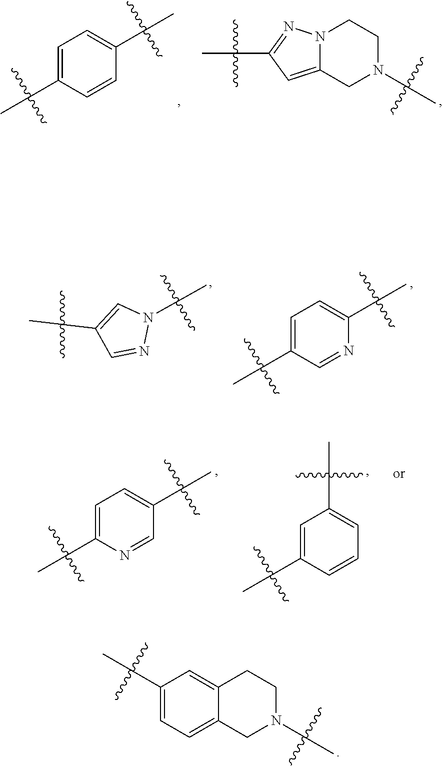

For example, ring A is selected from

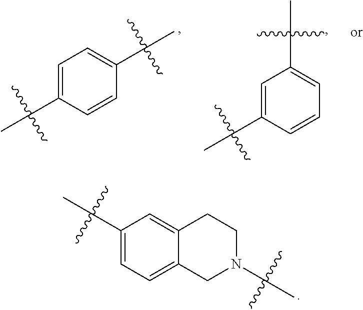 [SMILES: *c1ccc(*)cc1 ; *c1cccc(*)c1 ; *c1ccc2c(c1)CCN(*)C2]

In some embodiments, at least one of X¹, X², and X⁵ is —C(O)—N(R)— or —CH₂—.

In some embodiments, X¹ is —C(O)—N(R)—. In other examples, X¹ is —C₁₋₅ alkyl-; 7-12 membered spiro bicyclic heterocycloalkyl having 1-3 heteroatoms independently selected from N, O, or S wherein the heterocycloalkyl is optionally substituted with —CH₃; or 4-6 membered heterocycloalkyl having 1-2 heteroatoms independently selected from N, O, or S wherein the heterocycloalkyl is optionally substituted with —CH₃. In some examples, X¹ is —CH₂—, —C(O)—,

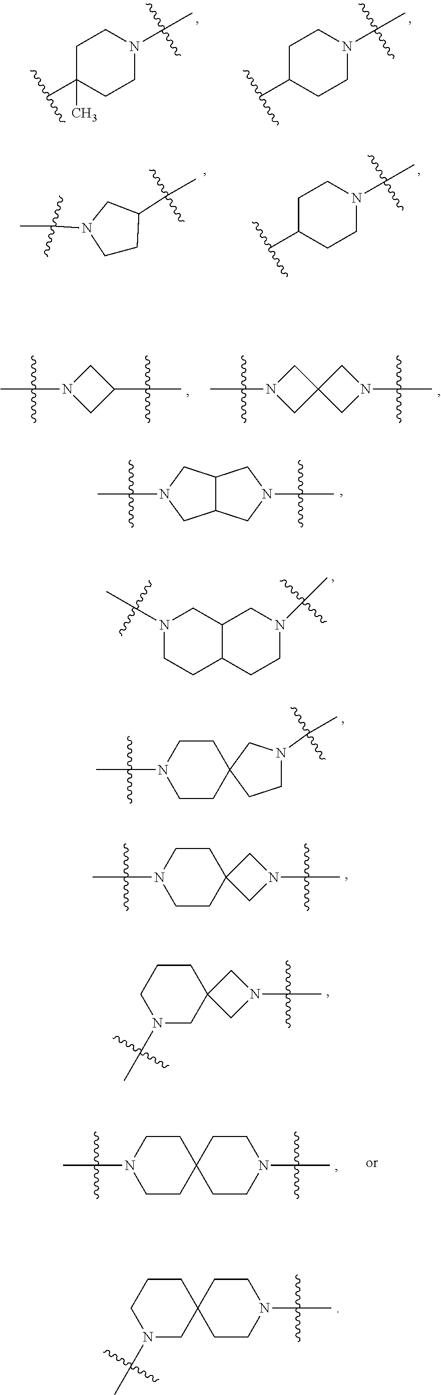

In some embodiments, X² is —(O—CH₂—CH₂)ₙ—, —(CH₂—CH₂—O)ₙ—, or —C₁₋₅ alkyl-. In other embodiment, X² is a bond, —C(O)—, —C₁₋₅ alkyl-,

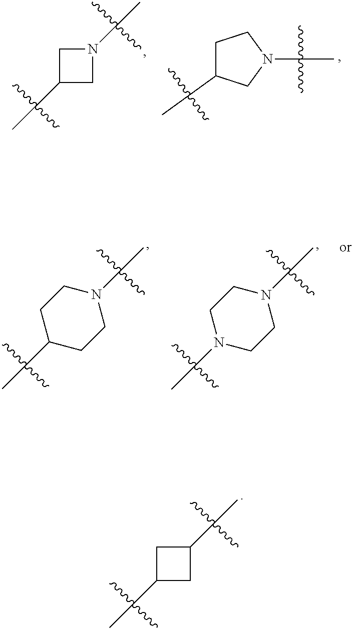 [SMILES: *C1CN(*)C1 ; *C1CCN(*)C1 ; *C1CCN(*)CC1 ; *N1CCN(*)CC1 ; *C1CC(*)C1]

For example, X² is a bond, —CH₂—, —CH₂—CH₂—, or —CH₂—CH₂—CH₂—.

In some embodiments, X³ is a bond, —C≡C—, —C₁₋₄ alkyl-, 4-6 membered cycloalkyl, 4-6 membered heterocycloalkyl having 1-2 heteroatoms independently selected from N, O, or S wherein the heterocycloalkyl is optionally substituted with —CH₃, or —N(R)—. In some examples, X³ is a bond, —C₁₋₄ alkyl-, or —N(R)—. In other embodiments, X³ is

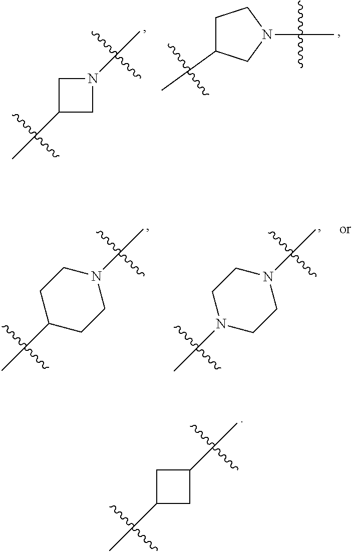 [SMILES: *C1CN(*)C1 ; *C1CCN(*)C1 ; *C1CCN(*)CC1 ; *N1CCN(*)CC1 ; *C1CC(*)C1]

In some embodiments, X⁴ is a bond, —C₁₋₄ alkyl-, —(O—CH₂—CH₂—CH₂)ₘ—, or 5-6 membered saturated, partially unsaturated, or fully unsaturated carbocycle having 0-3 heteroatoms independently selected from N, O, or S. For example, X⁴ is a bond,

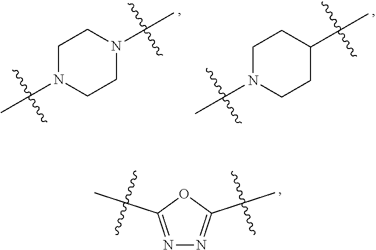 [SMILES: *N1CCN(*)CC1 ; *C1CCN(*)CC1 ; *c1nnc(*)o1]

—C₁₋₄ alkyl-, —CH₂—CH₂—N(R)—, or —N(R)—. In other examples, X⁴ is a bond, —CH₂—, or —N(R)—.

In some embodiments, X⁵ is a bond, —C₁₋₄ alkyl-, —N(R)—, or —C(O)—N(R)—. For example, X⁵ is a bond.

In some embodiments, X¹ is —(O—CH₂—CH₂—CH₂)ₘ—, m is 1, and X² is —C(O)—N(R)—.

In some embodiments, L is

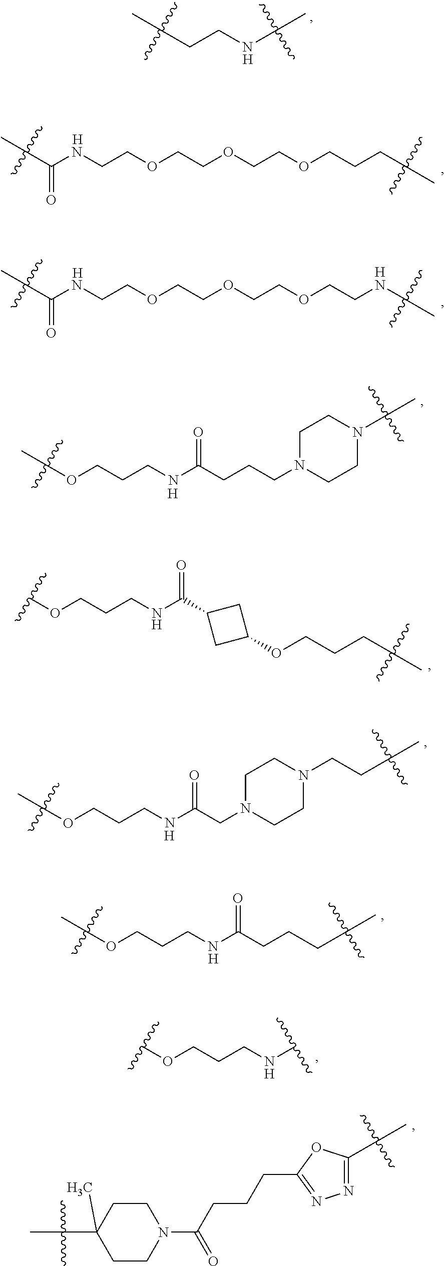

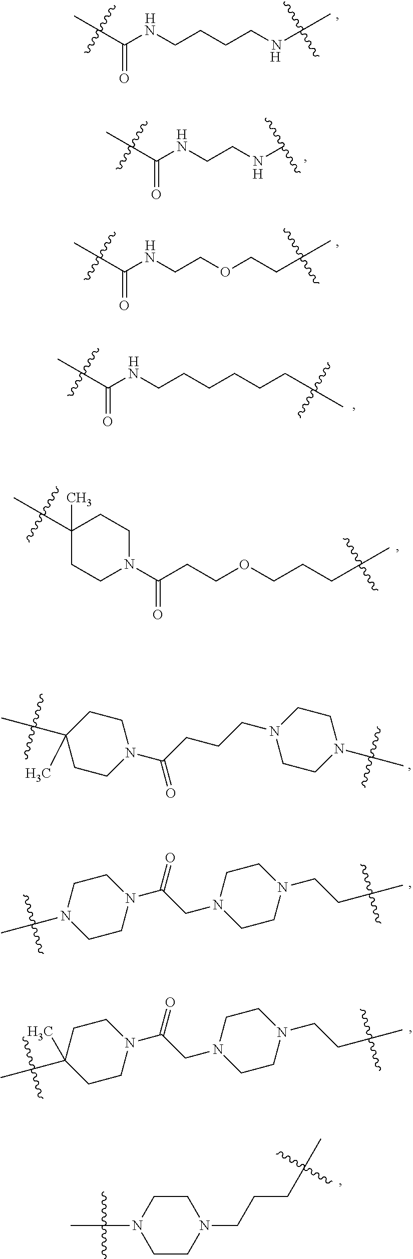

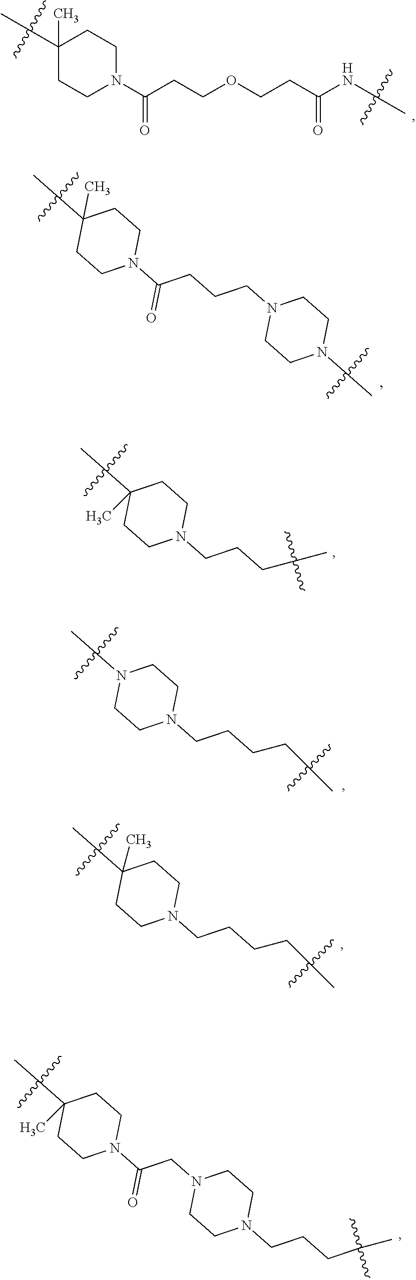

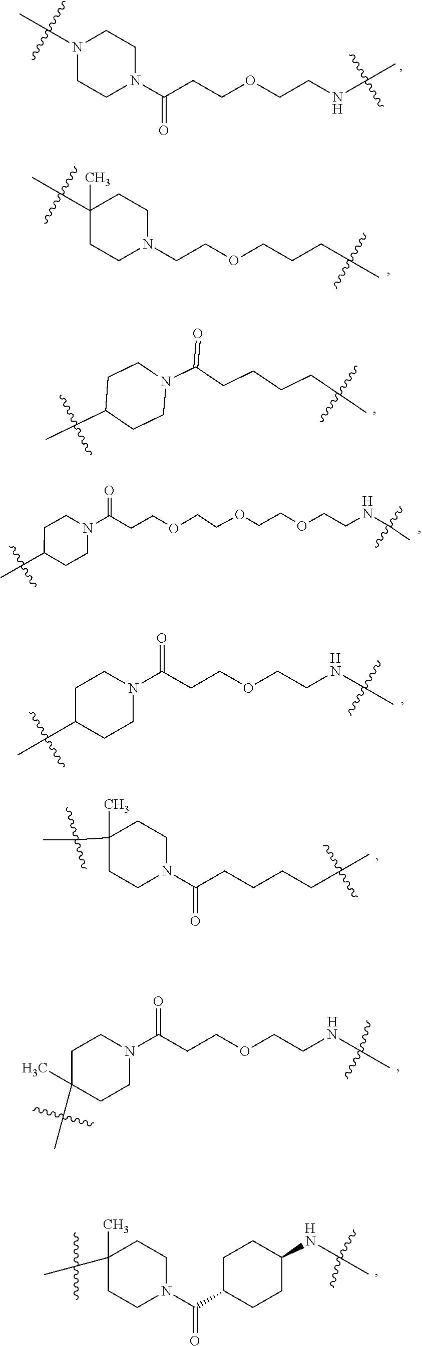

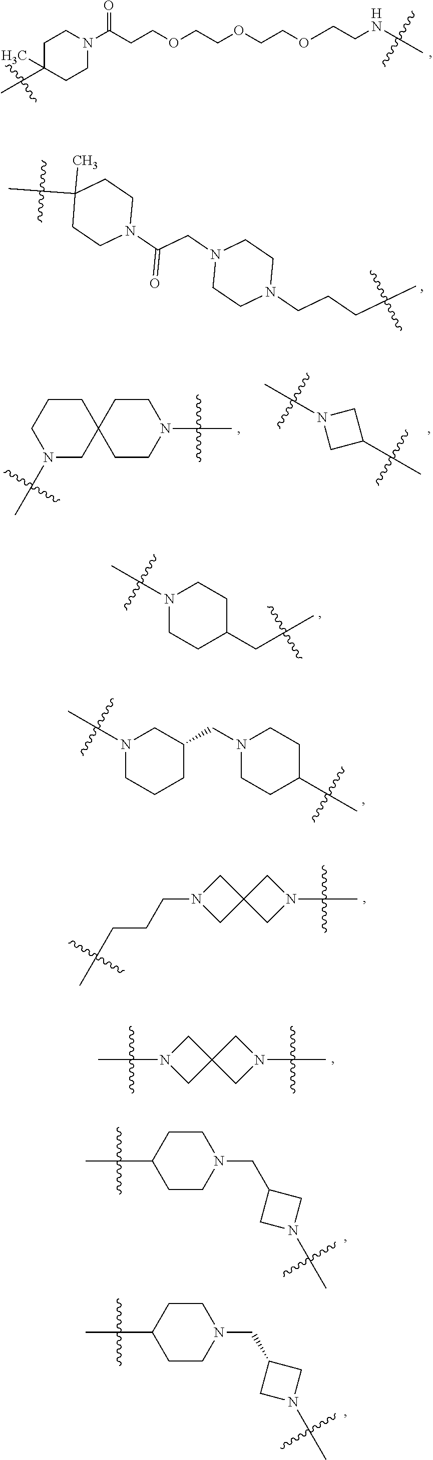

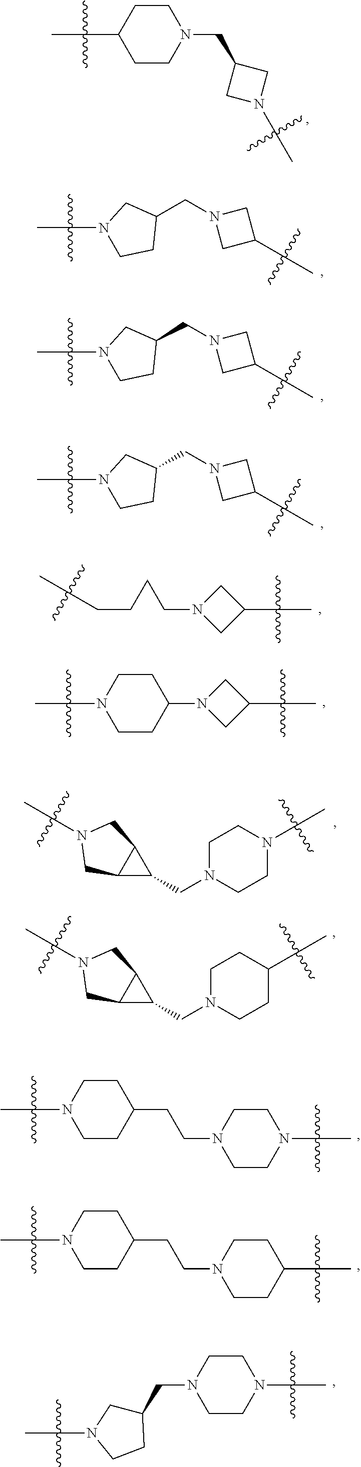

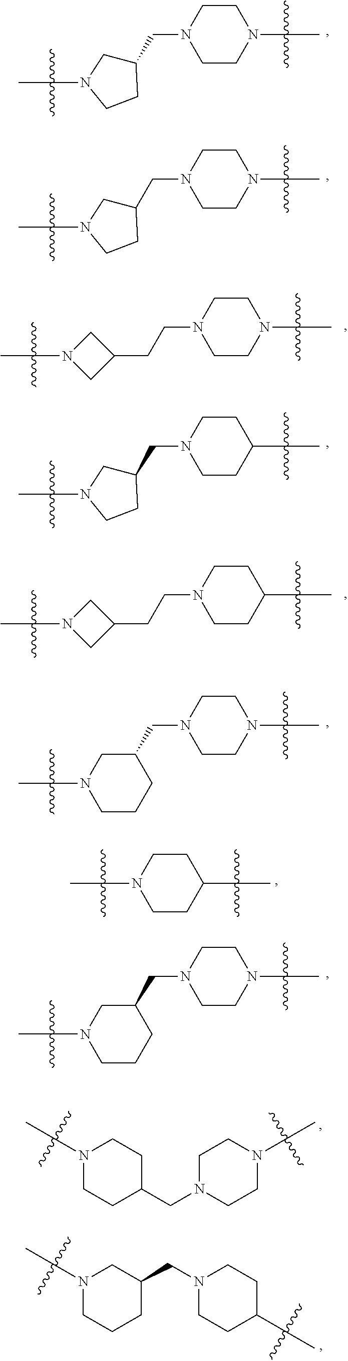

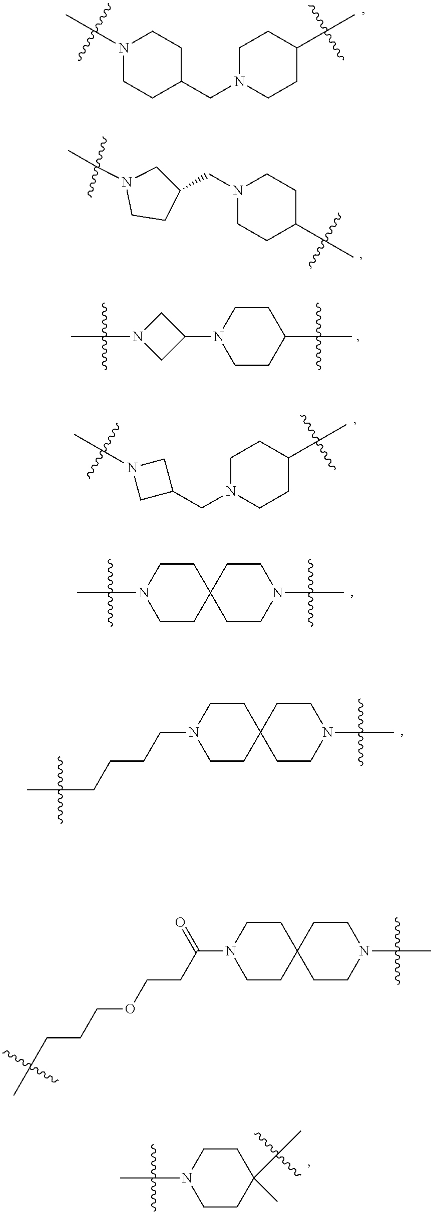

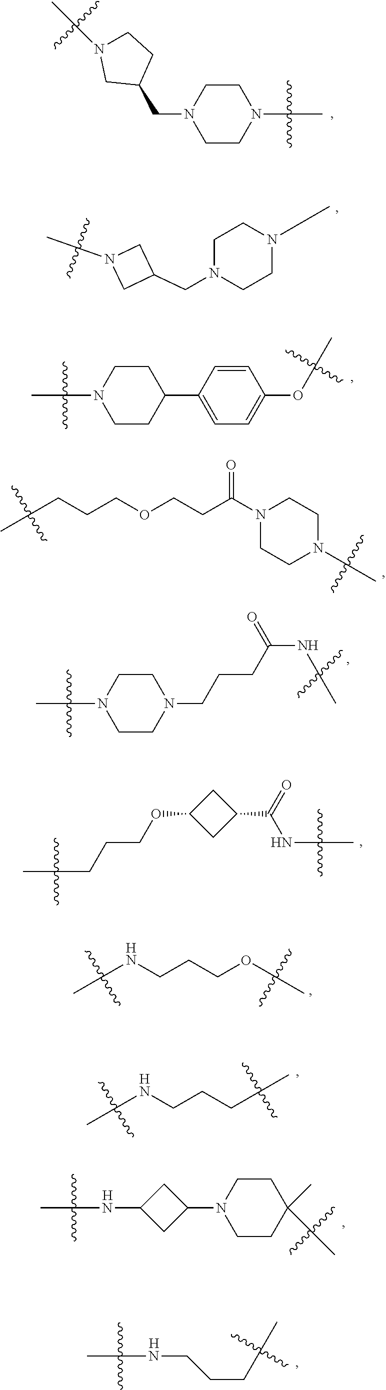

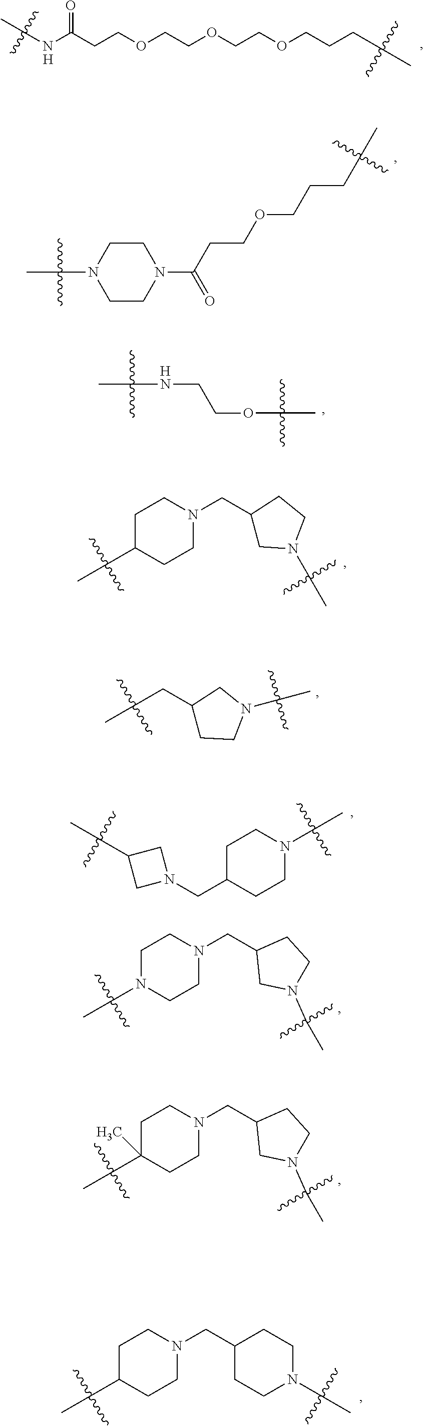

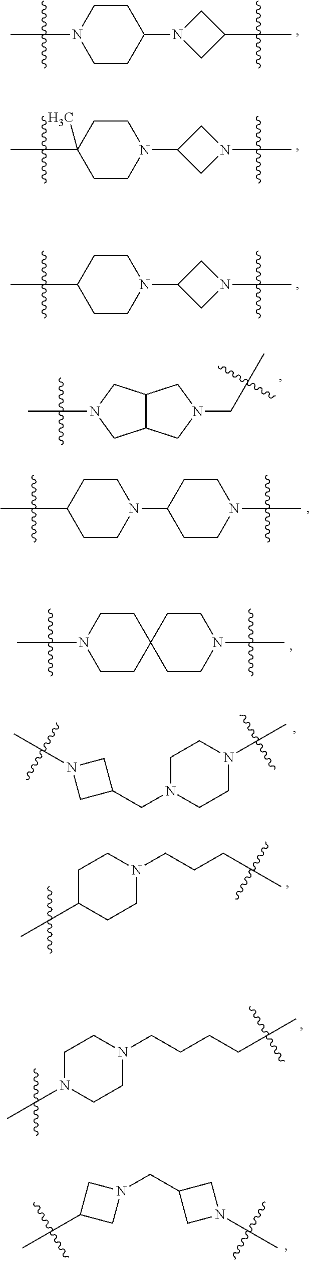

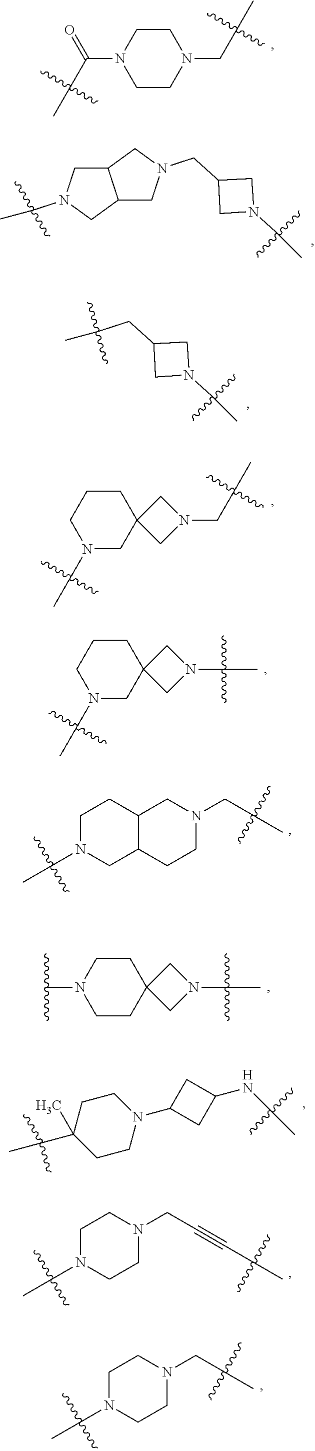

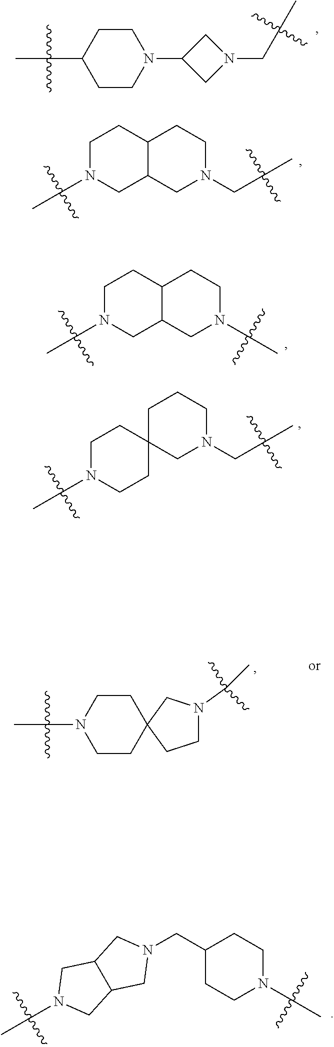

Another aspect of the present invention provides a compound of Formula (I)

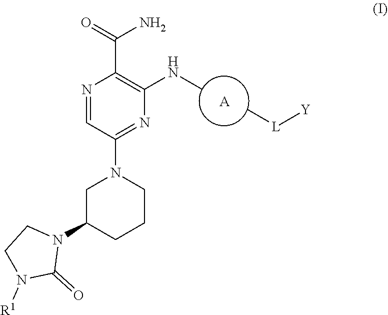

or a pharmaceutically acceptable salt thereof, wherein R¹ is C₁₋₃ alkyl; ring A is phenyl, 9-10 membered bicyclic aryl, or 9-10 membered bicyclic heteroaryl having 1-3 heteroatoms independently selected from N, O, or S; L is —X¹—X²—X³—X⁴—X⁵—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having 1-3 heteroatoms independently selected from N, O, or S wherein the heterocycloalkyl is optionally substituted with —CH₃, or 4-6 membered heterocycloalkyl having 1-2 heteroatoms independently selected from N, O, or S wherein the heterocycloalkyl is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having 1-2 heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, 4-6 membered heterocycloalkyl having 1-2 heteroatoms independently selected from N, O, or S wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having 1-3 heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from 1 to 3; Y is

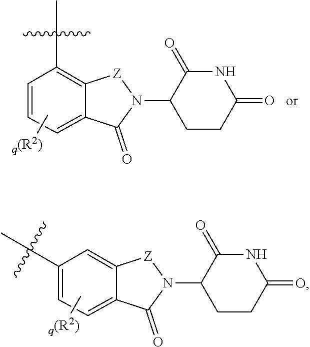

wherein each R² is independently halo or C₁₋₄ alkyl; each Z is —C(R^{A})₂— or —C(O)—; each R^{A} is independently —H or C₁₋₄ alkyl; and q is 0, 1, or 2.

In some embodiments, the compound of Formula (I) is a compound of Formula (I-A)

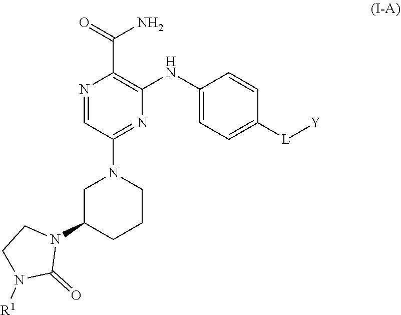

or a pharmaceutically acceptable salt thereof, wherein R¹ is C₁₋₃ alkyl; L is —X¹—X²—X³—X⁴—X⁵—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having 1-3 heteroatoms independently selected from N, O, or S wherein the heterocycloalkyl is optionally substituted with —CH₃, or 4-6 membered heterocycloalkyl having 1-2 heteroatoms independently selected from N, O, or S wherein the heterocycloalkyl is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having 1-2 heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, or 4-6 membered heterocycloalkyl having 1-2 heteroatoms independently selected from N, O, or S wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having 1-3 heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from 1 to 3; Y is

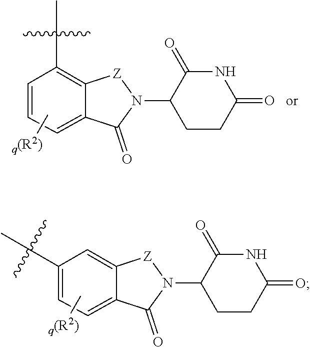

wherein each R² is independently halo or C₁₋₄ alkyl; each Z is —C(R^{A})₂- or —C(O)—; each R^{A} is independently —H or C₁₋₄ alkyl; and q is 0, 1, or 2.

In some embodiments, the compound of Formula (I) is a compound of Formula (I-B)

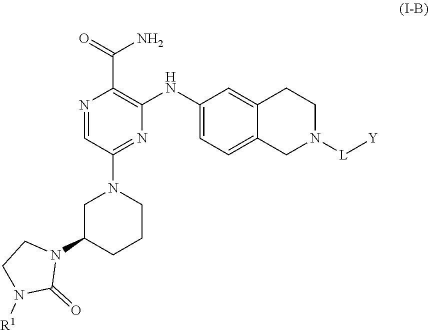

or a pharmaceutically acceptable salt thereof, wherein R¹ is C₁₋₃ alkyl; L is —X¹—X²—X³—X⁴—X⁵—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl ring having 1-3 heteroatoms independently selected from N, O, or S wherein the heterocycloalkyl is optionally substituted with —CH₃, or 4-6 membered heterocycloalkyl having 1-2 heteroatoms independently selected from N, O, or S wherein the heterocycloalkyl is optionally substituted with —CH₃; X² is a bond, —(O—CH₂—CH₂)ₙ—, —(CH₂—CH₂—O)—, —N(R)—C(O)—, —N(R)—, —C(O)—, —C₁₋₅ alkyl-, 4-6 membered cycloalkyl, or 4-6 membered heterocycloalkyl having 1-2 heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, or 4-6 membered heterocycloalkyl having 1-2 heteroatoms independently selected from N, O, or S wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having 1-3 heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from 1 to 3; Y is

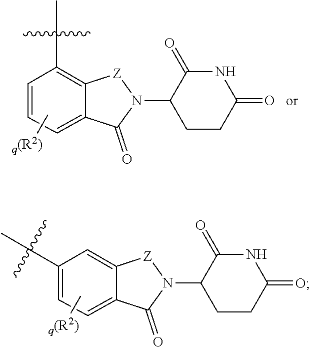

wherein each R² is independently halo or C₁₋₄ alkyl; each Z is —C(R^{A})₂— or —C(O)—; each R^{A} is independently —H or C₁₋₄ alkyl; and q is 0, 1, or 2.

In some embodiments, the compound of Formula (I) is a compound of Formula (II)

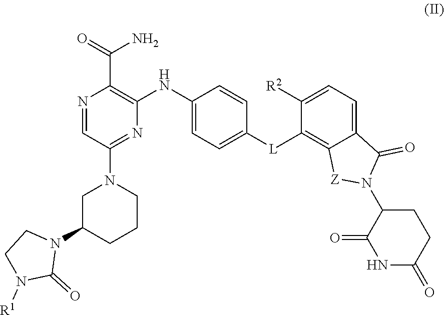

or a pharmaceutically acceptable salt thereof, wherein each of R¹, R², L, and Z are as defined in the compound of Formula (I).

In some embodiments, the compound of Formula (II) is a compound of Formulae (II-A) or (II-B)

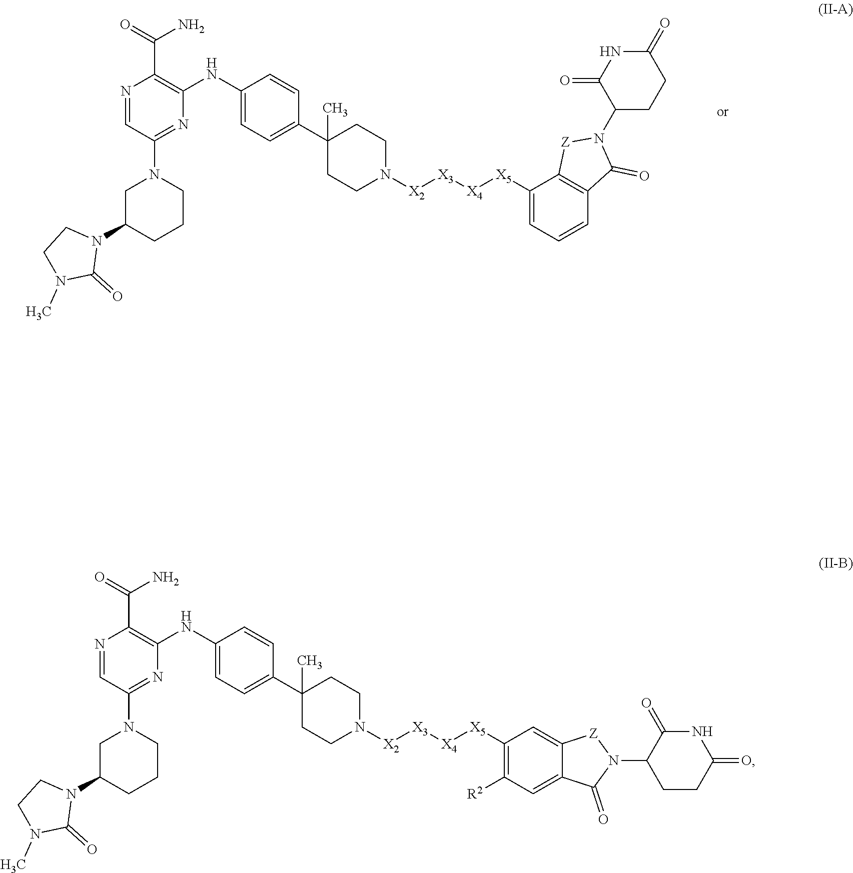

or a pharmaceutically acceptable salt thereof, wherein each of X², X³, X⁴, X⁵ and R² are as defined in the compound of Formula (I).

In some embodiments, the compound of Formula (I) is a compound of Formula (III)

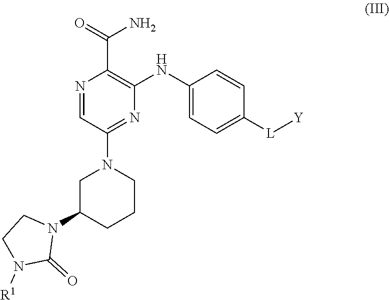

or a pharmaceutically acceptable salt thereof, wherein R¹ is C₁₋₃ alkyl; L is —X¹—X²—X³—; X¹ is 7-12 membered spiro bicyclic heterocycloalkyl having 1-3 heteroatoms independently selected from N, O, or S wherein the heterocycloalkyl is optionally substituted with —CH₃, or 4-6 membered heterocycloalkyl having 1-2 heteroatoms independently selected from N, O, or S wherein the heterocycloalkyl is optionally substituted with —CH₃; X² is a bond or —C₁₋₅ alkyl-; X³ is a bond, —C₁₋₄ alkyl-, or 4-6 membered heterocycloalkyl having 1-2 heteroatoms independently selected from N, O, or S wherein the heterocycloalkyl is optionally substituted with —CH₃; Y is

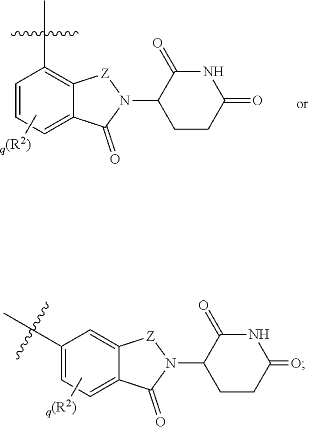

wherein each R² is independently halo or C₁₋₄ alkyl; each Z is —C(R^{A})₂— or —C(O)—; each R^{A} is independently —H; and q is 0, 1, or 2.

In some embodiments, the compound of Formula (I) is a compound of Formula (IV)

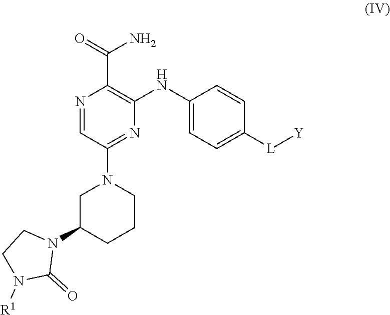

or a pharmaceutically acceptable salt thereof, wherein R¹ is C₁₋₃ alkyl; L is —X¹—X²—X³—X⁴—X⁵—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having 1-3 heteroatoms independently selected from N, O, or S wherein the heterocycloalkyl is optionally substituted with —CH₃, or 4-6 membered heterocycloalkyl having 1-2 heteroatoms independently selected from N, O, or S wherein the heterocycloalkyl is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having 1-2 heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, or 4-6 membered heterocycloalkyl having 1-2 heteroatoms independently selected from N, O, or S wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having 1-3 heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from 1 to 3; Y is

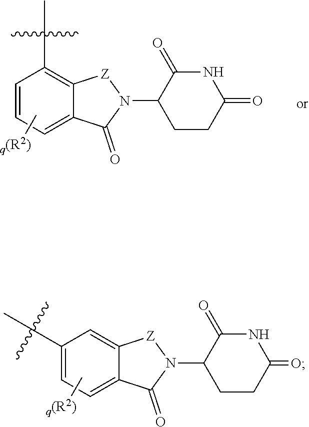

wherein each R² is independently halo or C₁₋₄ alkyl; each Z is —C(R^{A})₂— or —C(O)—; each R^{A} is independently —H or C₁₋₄ alkyl; and q is 0, 1, or 2.

In some embodiments, ring A is selected from

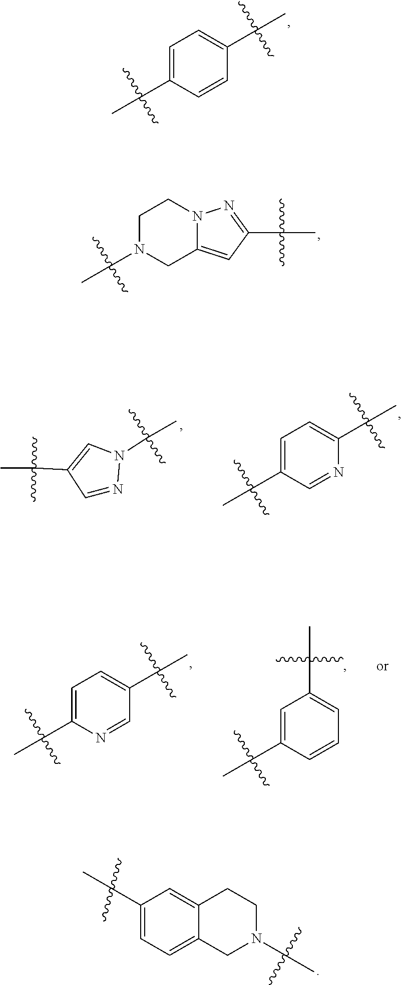

In some embodiments, Z is —CH— or —C(O)—.

In some embodiments, Y is

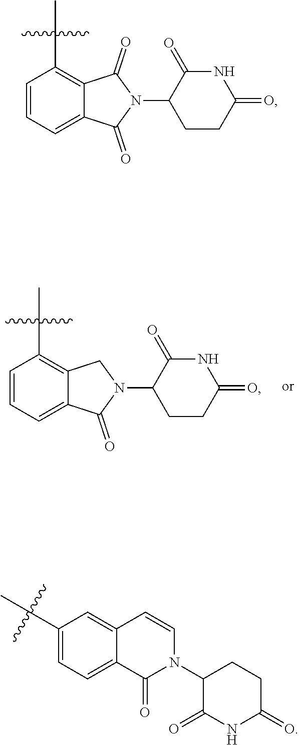 [SMILES: *c1cccc2c1C(=O)N(C1CCC(=O)NC1=O)C2=O ; *c1cccc2c1CN(C1CCC(=O)NC1=O)C2=O ; *c1ccc2c(=O)n(C3CCC(=O)NC3=O)ccc2c1]

In some embodiments, Y is

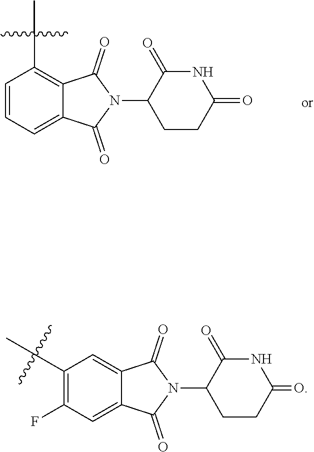 [SMILES: *c1cccc2c1C(=O)N(C1CCC(=O)NC1=O)C2=O ; *c1cc2c(cc1F)C(=O)N(C1CCC(=O)NC1=O)C2=O]

In some embodiments, R¹ is methyl, ethyl, or propyl.

In some embodiments, each R is independently —H or —CH₃.

In some embodiments, at least one of X¹, X², and X⁵ is —C(O)—N(R)—. For example, X¹ is —C(O)—N(R)—. In other examples, X² is —(O—CH₂—CH₂)ₙ—, —(CH₂—CH₂—O)ₙ—, or —C₁₋₅ alkyl-. And, in some examples, X³ is a bond, —C₁₋₄ alkyl-, or —N(R)—. In other examples, X⁴ is a bond or —N(R)—.

In some embodiments, X¹ is —(O—CH₂—CH₂—CH₂)ₘ—, m is 1, and X² is —C(O)—N(R)—.

In some embodiments, X³ is bond, —C₁₋₄ alkyl-, 4-6 membered cycloalkyl, or —N(R)—.

In some embodiments, X¹ is

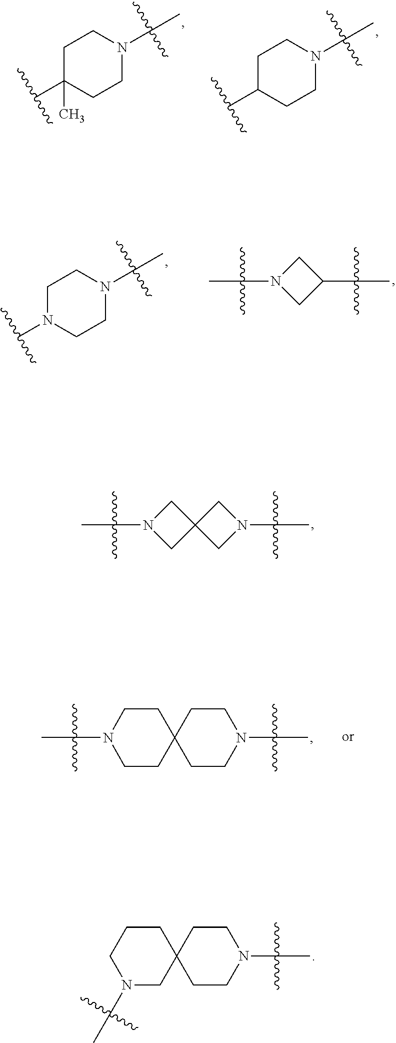

In some of these embodiments, X² is —C(O)—, —C₁₋₅ alkyl-, or 4-6 membered cycloalkyl. And, in some of these embodiments, X³ is a bond, —C₁₋₄ alkyl-, or —(CH₂—CH₂—O)ₚ—.

In some embodiments, X⁴ is a bond,

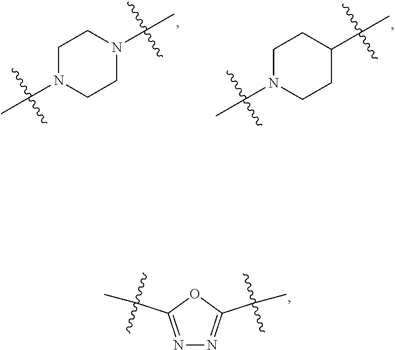 [SMILES: *N1CCN(*)CC1 ; *C1CCN(*)CC1 ; *c1nnc(*)o1]

—C₁₋₄ alkyl-, —CH₂—CH₂—N(R)—, or —N(R)—.

In some embodiments, X⁵ is a bond, —C₁₋₄ alkyl-, —N(R)—, or —C(O)—N(R)—.

In some embodiments, L is

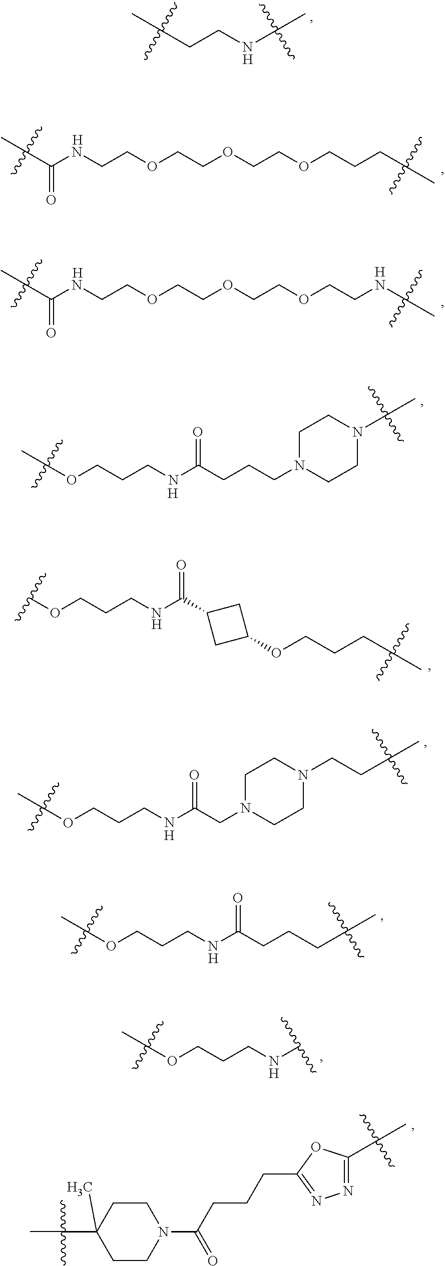

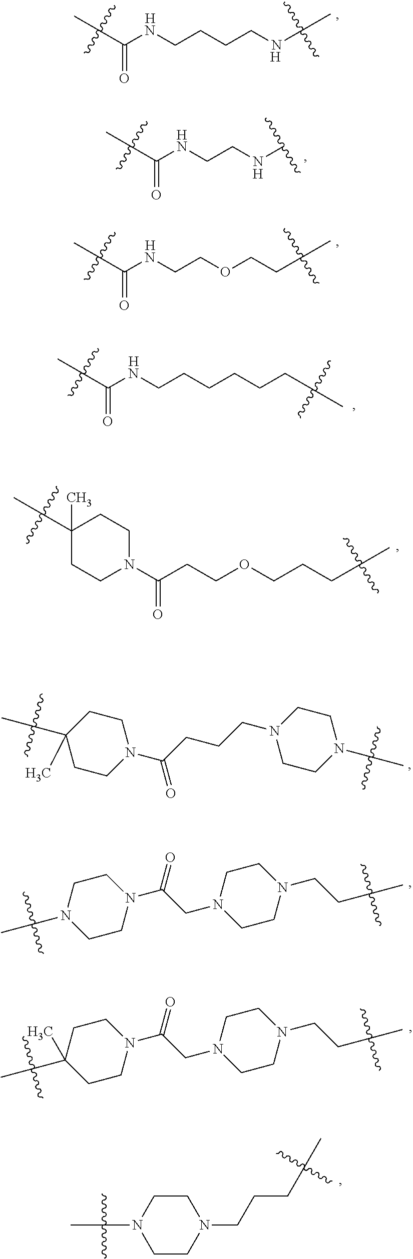

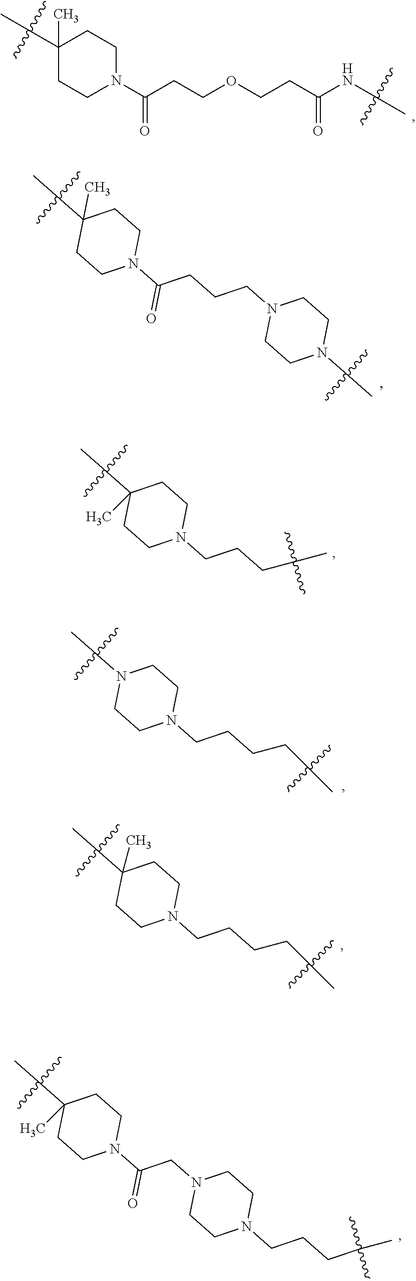

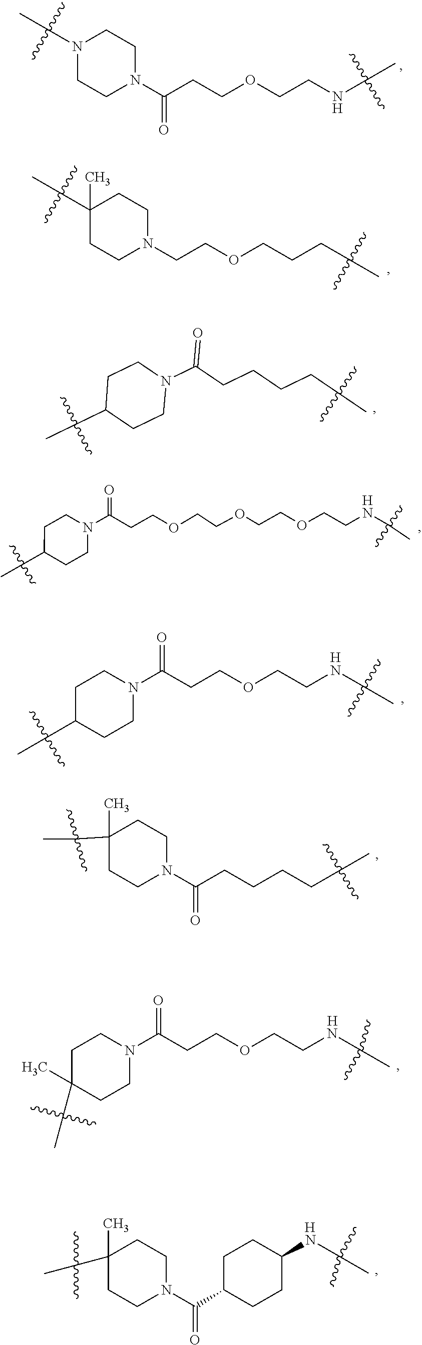

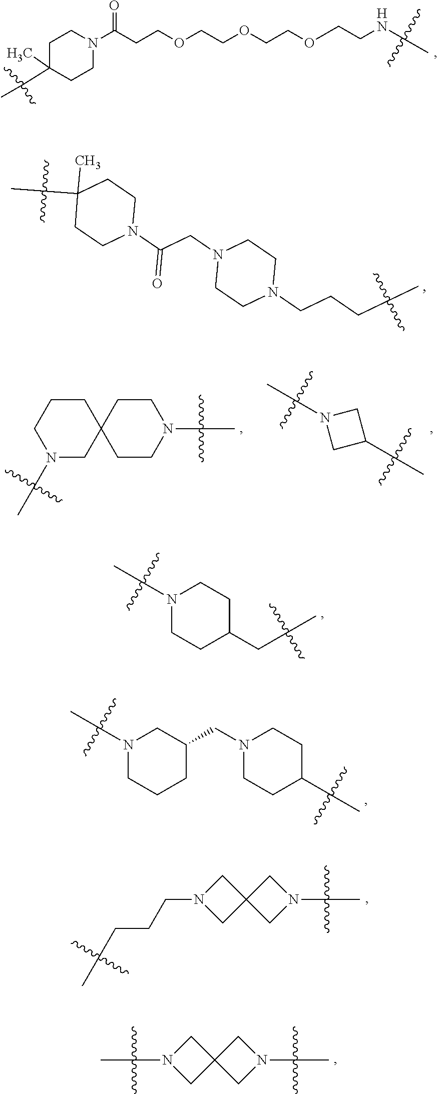

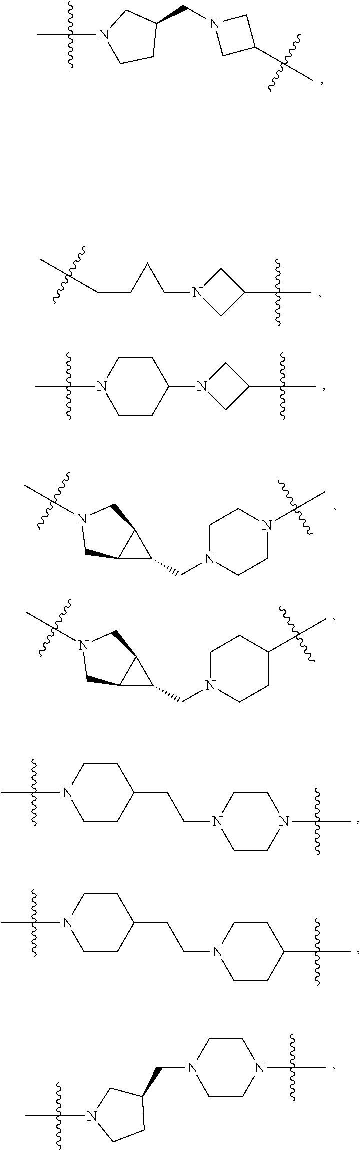

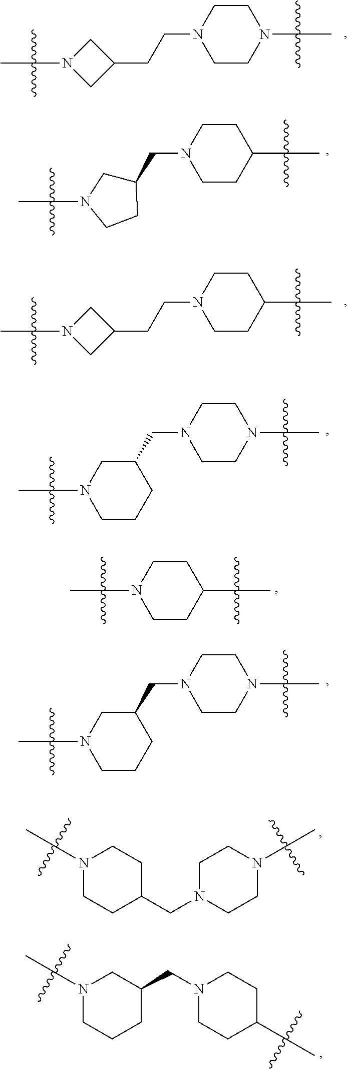

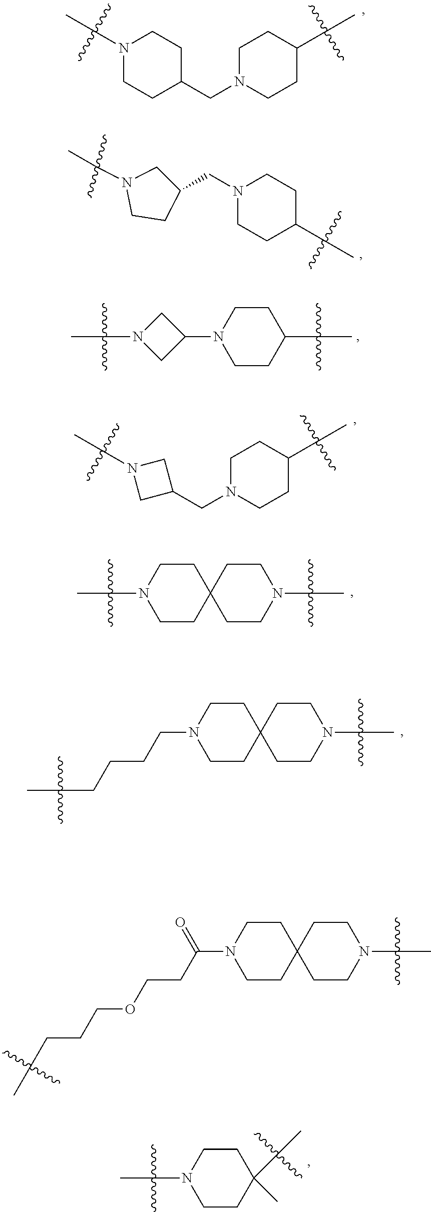

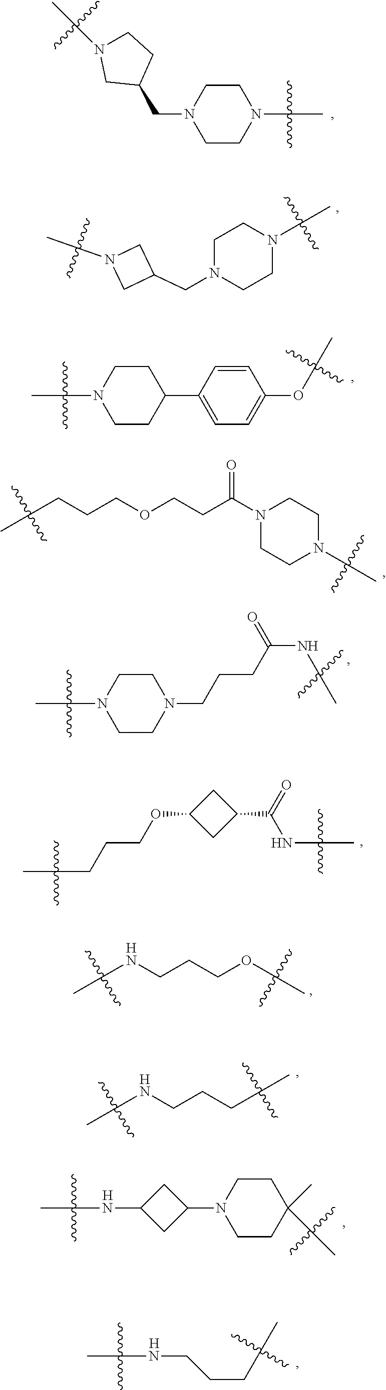

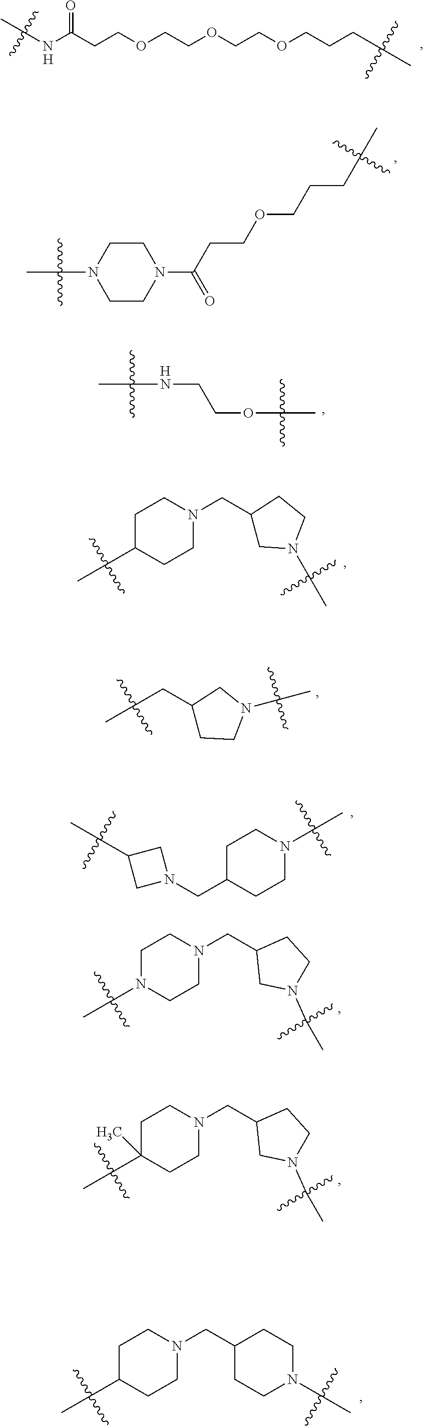

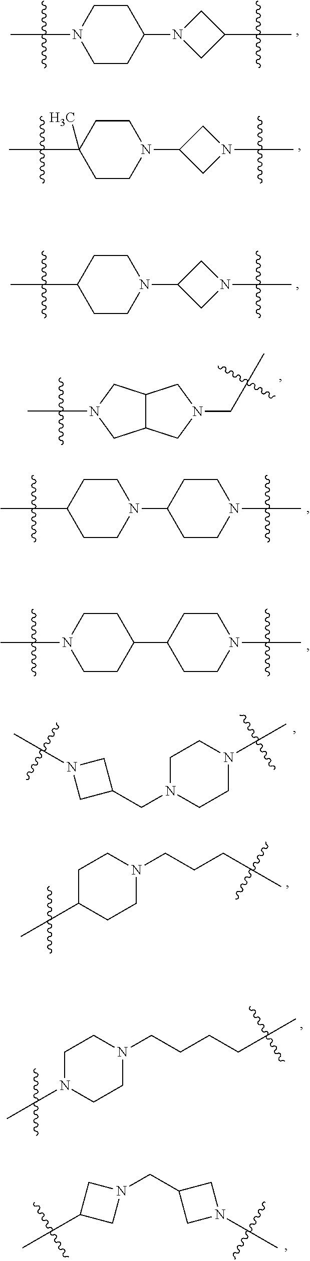

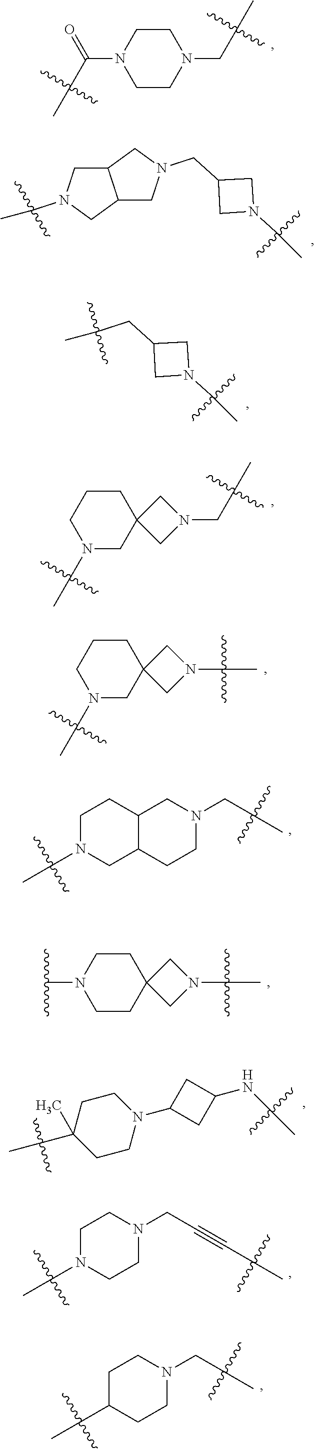

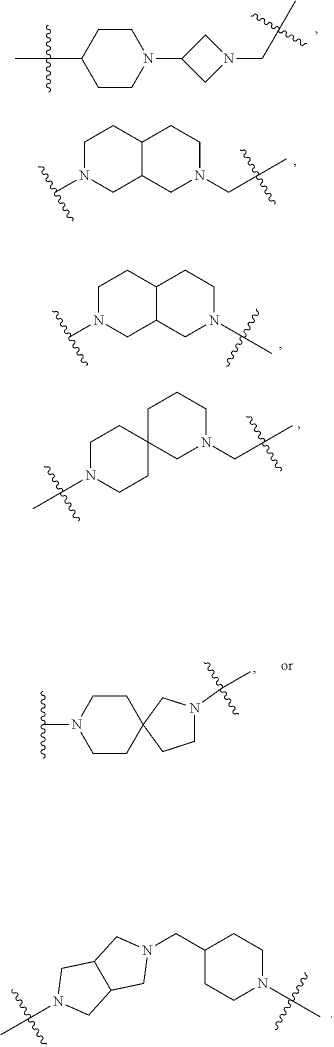

In some embodiments, the compound of Formula (I) is a compound of Formula (I-A)

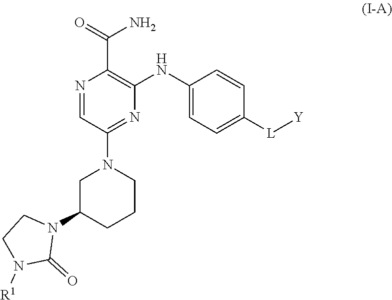

or a pharmaceutically acceptable salt thereof, wherein R¹ is C₁₋₃ alkyl; L is —X¹—X²—X³—X⁴—X⁵—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having 1-3 heteroatoms independently selected from N, O, or S wherein the heterocycloalkyl is optionally substituted with —CH₃, or 4-6 membered heterocycloalkyl having 1-2 heteroatoms independently selected from N, O, or S wherein the heterocycloalkyl is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having 1-2 heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, or 4-6 membered heterocycloalkyl having 1-2 heteroatoms independently selected from N, O, or S wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having 1-3 heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from 1 to 3; Y is

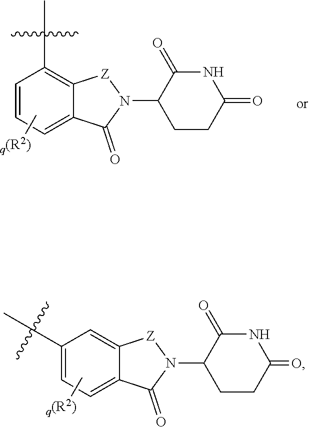

wherein each R² is independently halo or C₁₋₄ alkyl; each Z is —C(R^{A})₂— or —C(O)—; each R^{A} is independently —H or C₁₋₄ alkyl; and q is 0, 1, or 2.

In some embodiments, the compound of Formula (I) is a compound of Formula (I-B)

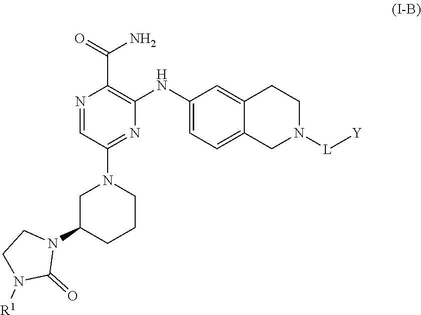

or a pharmaceutically acceptable salt thereof, wherein R¹ is C₁₋₃ alkyl; L is —X¹—X²—X³—X⁴—X⁵—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl ring having 1-3 heteroatoms independently selected from N, O, or S wherein the heterocycloalkyl is optionally substituted with —CH₃, or 4-6 membered heterocycloalkyl having 1-2 heteroatoms independently selected from N, O, or S wherein the heterocycloalkyl is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having 1-2 heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, or 4-6 membered heterocycloalkyl having 1-2 heteroatoms independently selected from N, O, or S wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having 1-3 heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from 1 to 3; Y is

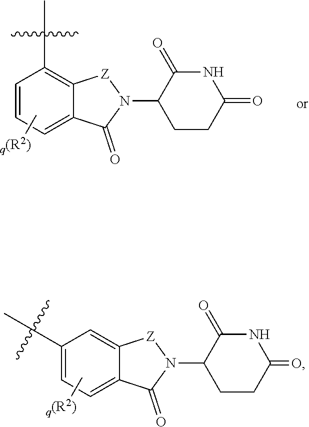

wherein each R² is independently halo or C₁₋₄ alkyl; each Z is —C(R^{A})₂— or —C(O)—; each R^{A} is independently —H or —C₁₋₄ alkyl; and q is 0, 1, or 2.

In some embodiments, the compound of Formula (I) is a compound of Formula (II)

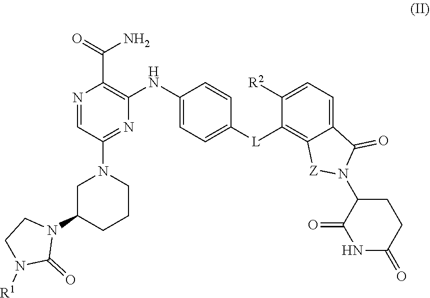

or a pharmaceutically acceptable salt thereof, wherein each of R¹, R², L, and Z are as defined herein for the compound of Formula (I), (I-A), or (I-B).

In some embodiments, the compound of Formula (I) is a compound of Formulae (II-A) or (II-B)

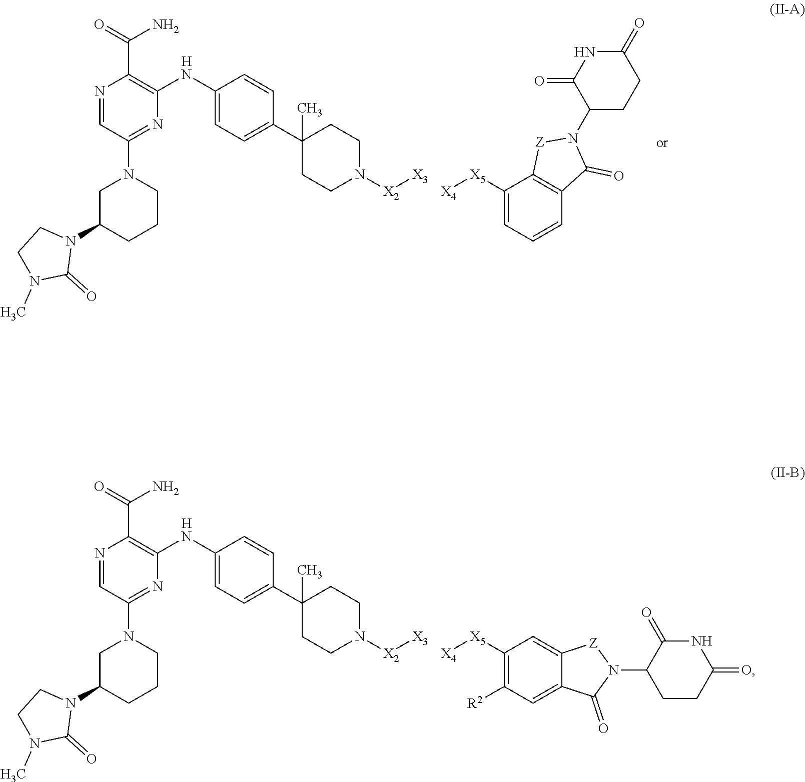

or a pharmaceutically acceptable salt thereof, wherein each of X², X³, X⁴, and X⁵ are as defined herein for the compound of Formula (I), (I-A), or (I-B).

In some embodiments, the compound of Formula (I) is a compound of Formula (III)

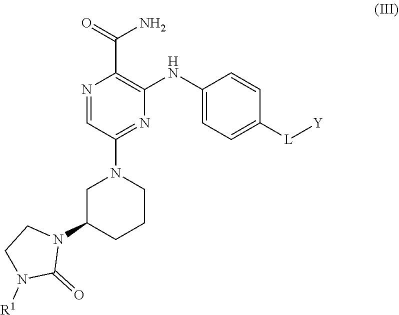

or a pharmaceutically acceptable salt thereof, wherein R¹ is —C₁₋₃ alkyl; L is —X¹—X²—X³—; X¹ is 7-12 membered spiro bicyclic heterocycloalkyl having 1-3 heteroatoms independently selected from N, O, or S wherein the heterocycloalkyl is optionally substituted with —CH₃, or 4-6 membered heterocycloalkyl having 1-2 heteroatoms independently selected from N, O, or S wherein the heterocycloalkyl is optionally substituted with —CH₃; X² is a bond or —C₁₋₅ alkyl-; X³ is a bond, —C₁₋₄ alkyl-,4-6 membered heterocycloalkyl having 1-2 heteroatoms independently selected from N, O, or S wherein the heterocycloalkyl is optionally substituted with —CH₃; Y is

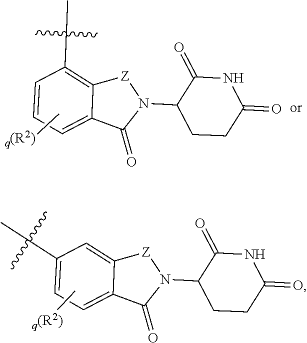

wherein each R² is independently halo or —C₁₋₄ alkyl; each Z is —C(R^{A})₂— or —C(O)—; each R^{A} is independently —H; and q is 0, 1, or 2.

In some embodiments, the compound of Formula (I) is a compound of Formula (IV)

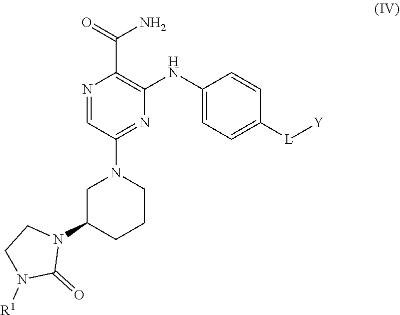

or a pharmaceutically acceptable salt thereof, wherein R¹ is C₁₋₃ alkyl; L is —X¹—X²—X³—X⁴—X⁵—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having 1-3 heteroatoms independently selected from N, O, or S wherein the heterocycloalkyl is optionally substituted with —CH₃, or 4-6 membered heterocycloalkyl having 1-2 heteroatoms independently selected from N, O, or S wherein the heterocycloalkyl is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having 1-2 heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, or 4-6 membered heterocycloalkyl having 1-2 heteroatoms independently selected from N, O, or S wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having 1-3 heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from 1 to 3; Y is

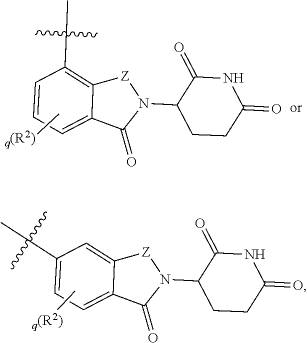

wherein each R² is independently halo or C₁₋₄ alkyl; each Z is —C(R^{A})₂— or —C(O)—; each R^{A} is independently —H or —C₁₋₄ alkyl; and q is 0, 1, or 2.

The present invention also provides a method of treating a disease or disorder mediated by BTK, comprising administering to a patient or biological sample a compound of Formula (A) or pharmaceutically acceptable salt thereof, or a pharmaceutical composition as described herein, wherein each of the variables contained therein are defined herein.

The present invention also provides a method of synthesizing a compound of Formula (A) or a pharmaceutically acceptable salt thereof.

### DETAILED DESCRIPTION

The present invention provides bifunctional compounds that induce the proteolytic degradation of BTK via a ubiquitin proteolysis pathway. The present invention also provides a compound of Formula (A) or a pharmaceutically acceptable salt thereof.

As used herein, the following definitions shall apply unless otherwise indicated.

### I. Definitions

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry,” Thomas Sorrell, University Science Books, Sausalito: 1999, and “March's Advanced Organic Chemistry,” 5th Ed., Ed.: Smith, M. B. and March, J., John Wiley & Sons, New York: 2001, the entire contents of which are hereby incorporated by reference.

As described herein, “protecting group” refers to a moiety or functionality that is introduced into a molecule by chemical modification of a functional group in order to obtain chemoselectivity in a subsequent chemical reaction. Standard protecting groups are provided in Wuts and Greene: “Greene's Protective Groups in Organic Synthesis,” 4th Ed, Wuts, P. G. M. and Greene, T. W., Wiley-Interscience, New York: 2006.

As described herein, compounds of the invention optionally may be substituted with one or more substituents, such as are illustrated generally above, or as exemplified by particular classes, subclasses, and species of the invention.

As used herein, the term “hydroxyl” or “hydroxy” refers to an —OH moiety.

As used herein the term “aliphatic” encompasses the terms alkyl, alkenyl, and alkynyl, each of which being optionally substituted as set forth below.

As used herein, an “alkyl” group refers to a saturated aliphatic hydrocarbon group containing 1-12 (e.g., 1-8, 1-6, or 1-4) carbon atoms. An alkyl group can be straight or branched. Examples of alkyl groups include, but are not limited to, methyl, ethyl, propyl, isopropyl, butyl, isobutyl, sec-butyl, tert-butyl, n-pentyl, n-heptyl, or 2-ethylhexyl. An alkyl group can be substituted (i.e., optionally substituted) with one or more substituents such as halo, phospho, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, or heterocycloaliphaticamino], sulfonyl [e.g., aliphatic-SO₂—], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroarylalkoxy, alkoxycarbonyl, alkylcarbonyloxy, or hydroxy. Without limitation, some examples of substituted alkyls include carboxyalkyl (such as HOOC-alkyl, alkoxycarbonylalkyl, and alkylcarbonyloxyalkyl), cyanoalkyl, hydroxyalkyl, alkoxyalkyl, acylalkyl, aralkyl, (alkoxyaryl)alkyl, (sulfonylamino)alkyl (such as (alkyl-SO₂-amino)alkyl), aminoalkyl, amidoalkyl, (cycloaliphatic)alkyl, or haloalkyl.

As used herein, an “alkenyl” group refers to an aliphatic carbon group that contains 2-8 (e.g., 2-12, 2-6, or 2-4) carbon atoms and at least one double bond. Like an alkyl group, an alkenyl group can be straight or branched. Examples of an alkenyl group include, but are not limited to allyl, 1- or 2-isopropenyl, 2-butenyl, and 2-hexenyl. An alkenyl group can be optionally substituted with one or more substituents such as halo, phospho, cycloaliphatic [e.g., cycloalkyl or cycloalkenyl], heterocycloaliphatic [e.g., heterocycloalkyl or heterocycloalkenyl], aryl, heteroaryl, alkoxy, aroyl, heteroaroyl, acyl [e.g., (aliphatic)carbonyl, (cycloaliphatic)carbonyl, or (heterocycloaliphatic)carbonyl], nitro, cyano, amido [e.g., (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino alkylaminocarbonyl, cycloalkylaminocarbonyl, heterocycloalkylaminocarbonyl, arylaminocarbonyl, or heteroarylaminocarbonyl], amino [e.g., aliphaticamino, cycloaliphaticamino, heterocycloaliphaticamino, or aliphaticsulfonylamino], sulfonyl [e.g., alkyl-SO₂—, cycloaliphatic-SO₂—, or aryl-SO₂—], sulfinyl, sulfanyl, sulfoxy, urea, thiourea, sulfamoyl, sulfamide, oxo, carboxy, carbamoyl, cycloaliphaticoxy, heterocycloaliphaticoxy, aryloxy, heteroaryloxy, aralkyloxy, heteroaralkoxy, alkoxycarbonyl, alkylcarbonyloxy, or hydroxy. Without limitation, some examples of substituted alkenyls include cyanoalkenyl, alkoxyalkenyl, acylalkenyl, hydroxyalkenyl, aralkenyl, (alkoxyaryl)alkenyl, (sulfonylamino)alkenyl (such as (alkyl-SO₂-amino)alkenyl), aminoalkenyl, amidoalkenyl, (cycloaliphatic)alkenyl, or haloalkenyl.

As used herein, an “alkynyl” group refers to an aliphatic carbon group that contains 2-8 (e.g., 2-12, 2-6, or 2-4) carbon atoms and has at least one triple bond. An alkynyl group can be straight or branched. Examples of an alkynyl group include, but are not limited to, propargyl and butynyl. An alkynyl group can be optionally substituted with one or more substituents such as aroyl, heteroaroyl, alkoxy, cycloalkyloxy, heterocycloalkyloxy, aryloxy, heteroaryloxy, aralkyloxy, nitro, carboxy, cyano, halo, hydroxy, sulfo, mercapto, sulfanyl [e.g., aliphaticsulfanyl or cycloaliphaticsulfanyl], sulfinyl [e.g., aliphaticsulfinyl or cycloaliphaticsulfinyl], sulfonyl [e.g., aliphatic-SO₂—, aliphaticamino-SO₂—, or cycloaliphatic-SO₂—], amido [e.g., aminocarbonyl, alkylaminocarbonyl, alkylcarbonylamino, cycloalkylaminocarbonyl, heterocycloalkylaminocarbonyl, cycloalkylcarbonylamino, arylaminocarbonyl, arylcarbonylamino, aralkylcarbonylamino, (heterocycloalkyl)carbonylamino, (cycloalkylalkyl)carbonylamino, heteroaralkylcarbonylamino, heteroarylcarbonylamino or heteroarylaminocarbonyl], urea, thiourea, sulfamoyl, sulfamide, alkoxycarbonyl, alkylcarbonyloxy, cycloaliphatic, heterocycloaliphatic, aryl, heteroaryl, acyl [e.g., (cycloaliphatic)carbonyl or (heterocycloaliphatic)carbonyl], amino [e.g., aliphaticamino], sulfoxy, oxo, carboxy, carbamoyl, (cycloaliphatic)oxy, (heterocycloaliphatic)oxy, or (heteroaryl)alkoxy.

As used herein, an “amido” encompasses both “aminocarbonyl” and “carbonylamino.” These terms when used alone or in connection with another group refer to an amido group such as —N(R^{X})—C(O)—R^{Y} or —C(O)—N(R^{X})₂, when used terminally, and —C(O)—N(R^{X})— or —N(R^{X})—C(O)— when used internally, wherein R^{X} and R^{Y} can be aliphatic, cycloaliphatic, aryl, araliphatic, heterocycloaliphatic, heteroaryl or heteroaraliphatic. Examples of amido groups include alkylamido (such as alkylcarbonylamino or alkylaminocarbonyl), (heterocycloaliphatic)amido, (heteroaralkyl)amido, (heteroaryl)amido, (heterocycloalkyl)alkylamido, arylamido, aralkylamido, (cycloalkyl)alkylamido, or cycloalkylamido.

As used herein, an “amino” group refers to —NR^{X}R^{Y} wherein each of R^{X} and R^{Y} is independently hydrogen, aliphatic, cycloaliphatic, (cycloaliphatic)aliphatic, aryl, araliphatic, heterocycloaliphatic, (heterocycloaliphatic)aliphatic, heteroaryl, carboxy, sulfanyl, sulfinyl, sulfonyl, (aliphatic)carbonyl, (cycloaliphatic)carbonyl, ((cycloaliphatic)aliphatic)carbonyl, arylcarbonyl, (araliphatic)carbonyl, (heterocycloaliphatic)carbonyl, ((heterocycloaliphatic)aliphatic)carbonyl, (heteroaryl)carbonyl, or (heteroaraliphatic)carbonyl, each of which being defined herein and being optionally substituted. Examples of amino groups include alkylamino, dialkylamino, or arylamino. When the term “amino” is not the terminal group (e.g., alkylcarbonylamino), it is represented by —NR^{X}—, where R^{X} has the same meaning as defined above.

As used herein, an “aryl” group used alone or as part of a larger moiety as in “aralkyl,” “aralkoxy,” or “aryloxyalkyl” refers to monocyclic (e.g., phenyl); bicyclic (e.g., indenyl, naphthalenyl, tetrahydronaphthyl, tetrahydroindenyl); and tricyclic (e.g., fluorenyl tetrahydrofluorenyl, or tetrahydroanthracenyl, anthracenyl) ring systems in which the monocyclic ring system is aromatic or at least one of the rings in a bicyclic or tricyclic ring system is aromatic. The bicyclic and tricyclic groups include benzofused 2-3 membered carbocyclic rings. For example, a benzofused group includes phenyl fused with two or more C₄₋₈ carbocyclic moieties. An aryl is optionally substituted with one or more substituents including aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic ring of a benzofused bicyclic or tricyclic aryl); nitro; carboxy; amido; acyl [e.g., (aliphatic)carbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl]; sulfonyl [e.g., aliphatic-SO₂— or amino-SO₂—]; sulfinyl [e.g., aliphatic-S(O)— or cycloaliphatic-S(O)—]; sulfanyl [e.g., aliphatic-S—]; cyano; halo; hydroxy; mercapto; sulfoxy; urea; thiourea; sulfamoyl; sulfamide; or carbamoyl. Alternatively, an aryl can be unsubstituted.

Non-limiting examples of substituted aryls include haloaryl [e.g., mono-, di (such as p,m-dihaloaryl), and (trihalo)aryl]; (carboxy)aryl [e.g., (alkoxycarbonyl)aryl, ((aralkyl)carbonyloxy)aryl, and (alkoxycarbonyl)aryl]; (amido)aryl [e.g., (aminocarbonyl)aryl, (((alkylamino)alkyl)aminocarbonyl)aryl, (alkylcarbonyl)aminoaryl, (arylaminocarbonyl)aryl, and (((heteroaryl)amino)carbonyl)aryl]; aminoaryl [e.g., ((alkylsulfonyl)amino)aryl or ((dialkyl)amino)aryl]; (cyanoalkyl)aryl; (alkoxy)aryl; (sulfamoyl)aryl [e.g., (aminosulfonyl)aryl]; (alkylsulfonyl)aryl; (cyano)aryl; (hydroxyalkyl)aryl; ((alkoxy)alkyl)aryl; (hydroxy)aryl, ((carboxy)alkyl)aryl; (((dialkyl)amino)alkyl)aryl; (nitroalkyl)aryl; (((alkylsulfonyl)amino)alkyl)aryl; ((heterocycloaliphatic)carbonyl)aryl; ((alkylsulfonyl)alkyl)aryl; (cyanoalkyl)aryl; (hydroxyalkyl)aryl; (alkylcarbonyl)aryl; alkylaryl; (trihaloalkyl)aryl; p-amino-m-alkoxycarbonylaryl; p-amino-m-cyanoaryl; p-halo-m-aminoaryl; or (m-(heterocycloaliphatic)-o-(alkyl))aryl.

As used herein, an “araliphatic” such as an “aralkyl” group refers to an aliphatic group (e.g., a C₁₋₄ alkyl group) that is substituted with an aryl group. “Aliphatic,” “alkyl,” and “aryl” are defined herein. An example of an araliphatic such as an aralkyl group is benzyl.

As used herein, an “aralkyl” group refers to an alkyl group (e.g., a C₁₋₄ alkyl group) that is substituted with an aryl group. Both “alkyl” and “aryl” have been defined above. An example of an aralkyl group is benzyl. An aralkyl is optionally substituted with one or more substituents such as aliphatic [e.g., alkyl, alkenyl, or alkynyl, including carboxyalkyl, hydroxyalkyl, or haloalkyl such as trifluoromethyl], cycloaliphatic [e.g., cycloalkyl or cycloalkenyl], (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, amido [e.g., aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, or heteroaralkylcarbonylamino], cyano, halo, hydroxy, acyl, mercapto, alkylsulfanyl, sulfoxy, urea, thiourea, sulfamoyl, sulfamide, oxo, or carbamoyl.

As used herein, a “bicyclic ring system” includes 6-12 (e.g., 8-12 or 9, 10, or 11) membered structures that form two rings, wherein the two rings have at least one atom in common (e.g., 2 atoms in common). Bicyclic ring systems include bicycloaliphatics (e.g., bicycloalkyl or bicycloalkenyl), bicycloheteroaliphatics, bicyclic aryls, and bicyclic heteroaryls.

As used herein, a “cycloaliphatic” group encompasses a “cycloalkyl” group and a “cycloalkenyl” group, each of which being optionally substituted as set forth below.

As used herein, a “cycloalkyl” group refers to a saturated carbocyclic mono- or bicyclic (fused or bridged) ring of 3-10 (e.g., 5-10) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, or ((aminocarbonyl)cycloalkyl)cycloalkyl.

A “cycloalkenyl” group, as used herein, refers to a non-aromatic carbocyclic ring of 3-10 (e.g., 4-8) carbon atoms having one or more double bonds. Examples of cycloalkenyl groups include cyclopentenyl, 1,4-cyclohexa-di-enyl, cycloheptenyl, cyclooctenyl, hexahydro-indenyl, octahydro-naphthyl, cyclohexenyl, bicyclo[2.2.2]octenyl, or bicyclo[3.3.1]nonenyl.

A cycloalkyl or cycloalkenyl group can be optionally substituted with one or more substituents such as phospho, aliphatic [e.g., alkyl, alkenyl, or alkynyl], cycloaliphatic, (cycloaliphatic) aliphatic, heterocycloaliphatic, (heterocycloaliphatic) aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic)aliphatic)carbonylamino, (aryl)carbonylamino, (araliphatic)carbonylamino, (heterocycloaliphatic)carbonylamino, ((heterocycloaliphatic)aliphatic)carbonylamino, (heteroaryl)carbonylamino, or (heteroaraliphatic)carbonylamino], nitro, carboxy [e.g., HOOC—, alkoxycarbonyl, or alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl], cyano, halo, hydroxy, mercapto, sulfonyl [e.g., alkyl-SO₂— and aryl-SO₂—], sulfinyl [e.g., alkyl-S(O)—], sulfanyl [e.g., alkyl-S—], sulfoxy, urea, thiourea, sulfamoyl, sulfamide, oxo, or carbamoyl.

As used herein, the term “heterocycloaliphatic” encompasses heterocycloalkyl groups and heterocycloalkenyl groups, each of which being optionally substituted as set forth below.

As used herein, a “heterocycloalkyl” group refers to a 3-10 membered mono- or bicylic (fused, bridged, or spiro) (e.g., 5- to 10-membered mono- or bicyclic) saturated ring structure, in which one or more of the ring atoms is a heteroatom (e.g., N, O, S, or combinations thereof). Non-limiting examples of a heterocycloalkyl group include piperidyl, piperazyl, tetrahydropyranyl, tetrahydrofuryl, 1,4-dioxolanyl, 1,4-dithianyl, 1,3-dioxolanyl, oxazolidyl, isoxazolidyl, morpholinyl, thiomorpholyl, octahydrobenzofuryl, octahydrochromenyl, octahydrothiochromenyl, octahydroindolyl, octahydropyrindinyl, decahydroquinolinyl, octahydrobenzo[b]thiopheneyl, 2-oxa-bicyclo[2.2.2]octyl, 1-aza-bicyclo[2.2.2]octyl, 3-aza-bicyclo[3.2.1]octyl, decahydro-2,7-naphthyridine, 2,8-diazaspiro[4.5]decane, 2,7-diazaspiro[3.5]nonane, octahydropyrrolo[3,4-c]pyrrole, octahydro-1H-pyrrolo[3,4-b]pyridine, and 2,6-dioxa-tricyclo[3.3.1.0^{3,7}]nonyl. A monocyclic heterocycloalkyl group can be fused with a phenyl moiety to form structures, such as tetrahydroisoquinoline, that would be categorized as heteroaryls.

A “heterocycloalkenyl” group, as used herein, refers to a mono- or bicylic (e.g., 5- to 10-membered mono- or bicyclic) non-aromatic ring structure having one or more double bonds, and wherein one or more of the ring atoms is a heteroatom (e.g., N, O, or S). Monocyclic and bicyclic heterocycloaliphatics are numbered according to standard chemical nomenclature.

A heterocycloalkyl or heterocycloalkenyl group can be optionally substituted with one or more substituents such as phospho, aliphatic [e.g., alkyl, alkenyl, or alkynyl], cycloaliphatic, (cycloaliphatic)aliphatic, heterocycloaliphatic, (heterocycloaliphatic)aliphatic, aryl, heteroaryl, alkoxy, (cycloaliphatic)oxy, (heterocycloaliphatic)oxy, aryloxy, heteroaryloxy, (araliphatic)oxy, (heteroaraliphatic)oxy, aroyl, heteroaroyl, amino, amido [e.g., (aliphatic)carbonylamino, (cycloaliphatic)carbonylamino, ((cycloaliphatic) aliphatic)carbonylamino, (aryl)carbonylamino, (araliphatic)carbonylamino, (heterocycloaliphatic)carbonylamino, ((heterocycloaliphatic) aliphatic)carbonylamino, (heteroaryl)carbonylamino, or (heteroaraliphatic)carbonylamino], nitro, carboxy [e.g., HOOC—, alkoxycarbonyl, or alkylcarbonyloxy], acyl [e.g., (cycloaliphatic)carbonyl, ((cycloaliphatic) aliphatic)carbonyl, (araliphatic)carbonyl, (heterocycloaliphatic)carbonyl, ((heterocycloaliphatic)aliphatic)carbonyl, or (heteroaraliphatic)carbonyl], nitro, cyano, halo, hydroxy, mercapto, sulfonyl [e.g., alkylsulfonyl or arylsulfonyl], sulfinyl [e.g., alkylsulfinyl], sulfanyl [e.g., alkylsulfanyl], sulfoxy, urea, thiourea, sulfamoyl, sulfamide, oxo, or carbamoyl.

A “heteroaryl” group, as used herein, refers to a monocyclic, bicyclic, or tricyclic ring system having 4 to 15 ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having 2 to 3 rings. For example, a benzofused group includes benzo fused with one or two 4 to 8 membered heterocycloaliphatic moieties (e.g., indolizyl, indolyl, isoindolyl, 3H-indolyl, indolinyl, benzo[b]furyl, benzo[b]thiophene-yl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl, cinnolyl, phthalazyl, quinazolyl, quinoxalyl, isoquinolyl, 4H-quinolizyl, benzo-1,2,5-thiadiazolyl, or 1,8-naphthyridyl. Other examples of heteroaryls include 1,2,3,4-tetrahydroisoquinoline and 4,5,6,7-tetrahydropyrazolo[1,5-a]pyrazine.

Without limitation, monocyclic heteroaryls include furyl, thiophene-yl, 2H-pyrrolyl, pyrrolyl, oxazolyl, thazolyl, imidazolyl, pyrazolyl, isoxazolyl, isothiazolyl, 1,3,4-thiadiazolyl, 2H-pyranyl, 4-H-pranyl, pyridyl, pyridazyl, pyrimidyl, pyrazolyl, pyrazyl, or 1,3,5-triazyl. Monocyclic heteroaryls are numbered according to standard chemical nomenclature.

Without limitation, bicyclic heteroaryls include indolizyl, indolyl, isoindolyl, 3H-indolyl, indolinyl, benzo[b]furyl, benzo[b]thiophenyl, quinolinyl, isoquinolinyl, indolizyl, isoindolyl, indolyl, benzo[b]furyl, bexo[b]thiophenyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature.

A heteroaryl is optionally substituted with one or more substituents such as aliphatic [e.g., alkyl, alkenyl, or alkynyl]; cycloaliphatic; (cycloaliphatic)aliphatic; heterocycloaliphatic; (heterocycloaliphatic)aliphatic; aryl; heteroaryl; alkoxy; (cycloaliphatic)oxy; (heterocycloaliphatic)oxy; aryloxy; heteroaryloxy; (araliphatic)oxy; (heteroaraliphatic)oxy; aroyl; heteroaroyl; amino; oxo (on a non-aromatic carbocyclic or heterocyclic ring of a bicyclic or tricyclic heteroaryl); carboxy; amido; acyl [e.g., aliphaticcarbonyl; (cycloaliphatic)carbonyl; ((cycloaliphatic)aliphatic)carbonyl; (araliphatic)carbonyl; (heterocycloaliphatic)carbonyl; ((heterocycloaliphatic)aliphatic)carbonyl; or (heteroaraliphatic)carbonyl]; sulfonyl [e.g., aliphaticsulfonyl or aminosulfonyl]; sulfinyl [e.g., aliphaticsulfinyl]; sulfanyl [e.g., aliphaticsulfanyl]; nitro; cyano; halo; hydroxy; mercapto; sulfoxy; urea; thiourea; sulfamoyl; sulfamide; or carbamoyl. Alternatively, a heteroaryl can be unsubstituted.

Non-limiting examples of substituted heteroaryls include (halo)heteroaryl [e.g., mono- and di-(halo)heteroaryl]; (carboxy)heteroaryl [e.g., (alkoxycarbonyl)heteroaryl]; cyanoheteroaryl; aminoheteroaryl [e.g., ((alkylsulfonyl)amino)heteroaryl and ((dialkyl)amino)heteroaryl]; (amido)heteroaryl [e.g., aminocarbonylheteroaryl, ((alkylcarbonyl)amino)heteroaryl, ((((alkyl)amino)alkyl)aminocarbonyl)heteroaryl, (((heteroaryl)amino)carbonyl)heteroaryl, ((heterocycloaliphatic)carbonyl)heteroaryl, and ((alkylcarbonyl)amino)heteroaryl]; (cyanoalkyl)heteroaryl; (alkoxy)heteroaryl; (sulfamoyl)heteroaryl [e.g., (aminosulfonyl)heteroaryl]; (sulfonyl)heteroaryl [e.g., (alkylsulfonyl)heteroaryl]; (hydroxyalkyl)heteroaryl; (alkoxyalkyl)heteroaryl; (hydroxy)heteroaryl; ((carboxy)alkyl)heteroaryl; (((dialkyl)amino)alkyl]heteroaryl; (heterocycloaliphatic)heteroaryl; (cycloaliphatic)heteroaryl; (nitroalkyl)heteroaryl; (((alkylsulfonyl)amino)alkyl)heteroaryl; ((alkylsulfonyl)alkyl)heteroaryl; (cyanoalkyl)heteroaryl; (acyl)heteroaryl [e.g., (alkylcarbonyl)heteroaryl]; (alkyl)heteroaryl; or (haloalkyl)heteroaryl [e.g., trihaloalkylheteroaryl].

As used herein, a “heteroaraliphatic” (such as a heteroaralkyl group) refers to an aliphatic group (e.g., a C₁₋₄ alkyl group) that is substituted with a heteroaryl group. “Aliphatic,” “alkyl,” and “heteroaryl” have been defined above.

As used herein, a “heteroaralkyl” group refers to an alkyl group (e.g., a C₁₋₄ alkyl group) that is substituted with a heteroaryl group. Both “alkyl” and “heteroaryl” have been defined above. A heteroaralkyl is optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, “cyclic moiety” and “cyclic group” refer to mono-, bi-, and tri-cyclic ring systems including cycloaliphatic, heterocycloaliphatic, aryl, or heteroaryl, each of which has been previously defined.

As used herein, a “bridged bicyclic ring system” refers to a bicyclic heterocyclicalipahtic ring system or bicyclic cycloaliphatic ring system in which the rings are bridged. Examples of bridged bicyclic ring systems include, but are not limited to, adamantanyl, norbornanyl, bicyclo[3.2.1]octyl, bicyclo[2.2.2]octyl, bicyclo[3.3.1]nonyl, bicyclo[3.3.2]decyl, 2-oxabicyclo[2.2.2]octyl, 1-azabicyclo[2.2.2]octyl, 3-azabicyclo[3.2.1]octyl, and 2,6-dioxa-tricyclo[3.3.1.0^{3,7}]nonyl. A bridged bicyclic ring system can be optionally substituted with one or more substituents such as alkyl (including carboxyalkyl, hydroxyalkyl, and haloalkyl such as trifluoromethyl), alkenyl, alkynyl, cycloalkyl, (cycloalkyl)alkyl, heterocycloalkyl, (heterocycloalkyl)alkyl, aryl, heteroaryl, alkoxy, cycloalkyloxy, heterocycloalkyloxy, aryloxy, heteroaryloxy, aralkyloxy, heteroaralkyloxy, aroyl, heteroaroyl, nitro, carboxy, alkoxycarbonyl, alkylcarbonyloxy, aminocarbonyl, alkylcarbonylamino, cycloalkylcarbonylamino, (cycloalkylalkyl)carbonylamino, arylcarbonylamino, aralkylcarbonylamino, (heterocycloalkyl)carbonylamino, (heterocycloalkylalkyl)carbonylamino, heteroarylcarbonylamino, heteroaralkylcarbonylamino, cyano, halo, hydroxy, acyl, mercapto, alkylsulfanyl, sulfoxy, urea, thiourea, sulfamoyl, sulfamide, oxo, or carbamoyl.

As used herein, an “acyl” group refers to a formyl group or R^{X}—C(O)— (such as alkyl-C(O)—, also referred to as “alkylcarbonyl”) where R^{X} and “alkyl” have been defined previously. Acetyl and pivaloyl are examples of acyl groups.

As used herein, an “aroyl” or “heteroaroyl” refers to an aryl-C(O)— or a heteroaryl-C(O)—. The aryl and heteroaryl portion of the aroyl or heteroaroyl is optionally substituted as previously defined.

As used herein, an “alkoxy” group refers to an alkyl-O— group where “alkyl” has been defined previously.

As used herein, a “carbamoyl” group refers to a group having the structure —O—CO—NR^{X}R^{Y} or —NR^{X}—CO—O—R^{Z}, wherein R^{X} and R^{Y} have been defined above and R^{Z} can be aliphatic, aryl, araliphatic, heterocycloaliphatic, heteroaryl, or heteroaraliphatic.

As used herein, a “carboxy” group refers to —COOH, —COOR^{X}, —OC(O)H, —OC(O)R^{X}, when used as a terminal group; or —OC(O)— or —C(O)O— when used as an internal group.

As used herein, a “haloaliphatic” group refers to an aliphatic group substituted with 1-3 halogen. For instance, the term haloalkyl includes the group —CF₃.

As used herein, a “mercapto” group refers to —SH.

As used herein, a “sulfo” group refers to —SO₃H or —SO₃R^{X} when used terminally or —S(O)₃— when used internally.

As used herein, a “sulfamide” group refers to the structure —NR^{X}—S(O)₂—NR^{Y}R^{Z} when used terminally and —NR^{X}—S(O)₂—NR^{Y}— when used internally, wherein R^{X}, R^{Y}, and R^{Z} have been defined above.

As used herein, a “sulfamoyl” group refers to the structure —O—S(O)₂—NR^{Y}R^{Z} wherein R^{Y} and R^{Z} have been defined above.

As used herein, a “sulfonamide” group refers to the structure —S(O)₂—NR^{X}R^{Y} or —NR^{X}—S(O)₂—R^{Z} when used terminally; or —S(O)₂—NR^{X}— or —NR^{X}—S(O)₂— when used internally, wherein R^{X}, R^{Y}, and R^{Z} are defined above.

As used herein a “sulfanyl” group refers to —S-R^{X} when used terminally and —S—when used internally, wherein R^{X} has been defined above. Examples of sulfanyls include aliphatic-S—, cycloaliphatic-S—, aryl-S—, or the like.

As used herein a “sulfinyl” group refers to —S(O)—R^{X} when used terminally and —S(O)— when used internally, wherein R^{X} has been defined above. Examples of sulfinyl groups include aliphatic-S(O)—, aryl-S(O)—, (cycloaliphatic(aliphatic))-S(O)—, cycloalkyl-S(O)—, heterocycloaliphatic-S(O)—, heteroaryl-S(O)—, or the like.

As used herein, a “sulfonyl” group refers to —S(O)₂—R^{X} when used terminally and —S(O)₂— when used internally, wherein R^{X} has been defined above. Examples of sulfonyl groups include aliphatic-S(O)₂—, aryl-S(O)₂—, (cycloaliphatic(aliphatic))-S(O)₂—, cycloaliphatic-S(O)₂—, heterocycloaliphatic-S(O)₂—, heteroaryl-S(O)₂—, (cycloaliphatic(amido(aliphatic)))-S(O)₂— or the like.

As used herein, a “sulfoxy” group refers to —O—S(O)—R^{X} or —S(O)—O-R^{X}, when used terminally and —O—S(O)— or —S(O)—O— when used internally, where R^{X} has been defined above.

As used herein, a “halogen” or “halo” group refers to fluorine, chlorine, bromine or iodine.

As used herein, an “alkoxycarbonyl,” which is encompassed by the term carboxy, used alone or in connection with another group refers to a group such as alkyl-O—C(O)—.

As used herein, an “alkoxyalkyl” refers to an alkyl group such as alkyl-O-alkyl-, wherein alkyl has been defined above.

As used herein, a “carbonyl” refers to —C(O)—.

As used herein, an “oxo” refers to ═O.

As used herein, the term “phospho” refers to phosphinates and phosphonates. Examples of phosphinates and phosphonates include —P(O)(R^{P})₂, wherein R^{P} is aliphatic, alkoxy, aryloxy, heteroaryloxy, (cycloaliphatic)oxy, (heterocycloaliphatic)oxy aryl, heteroaryl, cycloaliphatic or amino.

As used herein, an “aminoalkyl” refers to the structure (R^{X})₂N-alkyl-.

As used herein, a “cyanoalkyl” refers to the structure (NC)-alkyl-.

As used herein, a “urea” group refers to the structure —NR^{X}—CO—NR^{Y}R^{Z} and a “thiourea” group refers to the structure —NR^{X}—CS—NR^{Y}R^{Z} when used terminally and —NR^{X}—CO—NR^{Y}— or —NR^{X}—CS—NR^{Y}— when used internally, wherein R^{X}, R^{Y}, and R^{Z} have been defined above.

As used herein, a “guanidine” group refers to the structure —N═C(N(R^{X}R^{Y}))N(R^{X}R^{Y}) or —NR^{X}—C(═NR^{X})NR^{X}R^{Y} wherein R^{X} and R^{Y} have been defined above.

As used herein, the term “amidino” group refers to the structure —C═(NR^{X})N(R^{X}R^{Y}) wherein R^{X} and R^{Y} have been defined above.

As used herein, the term “vicinal” generally refers to the placement of substituents on a group that includes two or more carbon atoms, wherein the substituents are attached to adjacent carbon atoms.

As used herein, the term “geminal” generally refers to the placement of substituents on a group that includes two or more carbon atoms, wherein the substituents are attached to the same carbon atom.

The terms “terminally” and “internally” refer to the location of a group within a substituent. A group is terminal when the group is present at the end of the substituent not further bonded to the rest of the chemical structure. Carboxyalkyl, i.e., R^{X}O(O)C-alkyl, is an example of a carboxy group used terminally. A group is internal when the group is present in the middle of a substituent of the chemical structure. Alkylcarboxy (e.g., alkyl-C(O)O— or alkyl-OC(O)—) and alkylcarboxyaryl (e.g., alkyl-C(O)O-aryl- or alkyl-O(CO)-aryl-) are examples of carboxy groups used internally.

As used herein, an “aliphatic chain” refers to a branched or straight aliphatic group (e.g., alkyl groups, alkenyl groups, or alkynyl groups). A straight aliphatic chain has the structure —[CH₂]ᵥ—, where v is 1-12. A branched aliphatic chain is a straight aliphatic chain that is substituted with one or more aliphatic groups. A branched aliphatic chain has the structure —[CQQ]ᵥ— where Q is independently a hydrogen or an aliphatic group; however, Q shall be an aliphatic group in at least one instance. The term aliphatic chain includes alkyl chains, alkenyl chains, and alkynyl chains, where alkyl, alkenyl, and alkynyl are defined above.

The phrase “optionally substituted” is used herein interchangeably with the phrase “substituted or unsubstituted.” As described herein, compounds of the invention can optionally be substituted with one or more substituents, such as are illustrated generally above, or as exemplified by particular classes, subclasses, and species of the invention. As described herein, the variables R, R¹, R², L, Y, and Z, and other variables contained in Formula (A), (B), (C), (D), (E), (F), (G), (H), (J), (K), (M), (X), (I), (I-A), (I-B), (II), (II-A), (II-B), (III), and (IV) described herein encompass specific groups, such as alkyl and aryl. Unless otherwise noted, each of the specific groups for the variables R, R¹⁰, R^{A}, R¹, R², L, L¹, D, W, E, V, G, Y, and Z, and other variables contained therein can be optionally substituted with one or more substituents described herein. Each substituent of a specific group is further optionally substituted with one to three of halo, cyano, oxo, alkoxy, hydroxy, amino, nitro, aryl, cycloaliphatic, heterocycloaliphatic, heteroaryl, haloalkyl, and alkyl. For instance, an alkyl group can be substituted with alkylsulfanyl and the alkylsulfanyl can be optionally substituted with one to three of halo, cyano, oxo, alkoxy, hydroxy, amino, nitro, aryl, haloalkyl, and alkyl. As an additional example, the cycloalkyl portion of a (cycloalkyl)carbonylamino can be optionally substituted with one to three of halo, cyano, alkoxy, hydroxy, nitro, haloalkyl, and alkyl. When two alkoxy groups are bound to the same atom or adjacent atoms, the two alkxoy groups can form a ring together with the atom(s) to which they are bound.

As used herein, the term “substituted,” whether preceded by the term “optionally” or not, refers generally to the replacement of hydrogen atoms in a given structure with the radical of a specified substituent. Specific substituents are described above in the definitions and below in the description of compounds and examples thereof. Unless otherwise indicated, an optionally substituted group can have a substituent at each substitutable position of the group, and when more than one position in any given structure can be substituted with more than one substituent selected from a specified group, the substituent can be either the same or different at every position. A ring substituent, such as a heterocycloalkyl, can be bound to another ring, such as a cycloalkyl, to form a spiro-bicyclic ring system, e.g., both rings share one common atom. Non-limiting examples of spiro heterocycloalkyls include

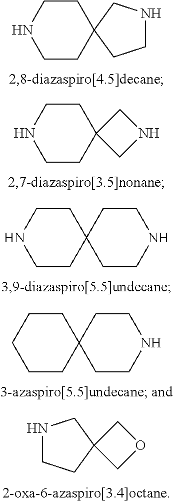

As one of ordinary skill in the art will recognize, combinations of substituents envisioned by this invention are those combinations that result in the formation of stable or chemically feasible compounds.

As used herein, the phrase “stable or chemically feasible” refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

As used herein, an “effective amount” is defined as the amount required to confer a therapeutic effect on the treated patient, and is typically determined based on age, surface area, weight, and condition of the patient. The interrelationship of dosages for animals and humans (based on milligrams per meter squared of body surface) is described by Freireich et al., Cancer Chemother. Rep., 50: 219 (1966). Body surface area may be approximately determined from height and weight of the patient. See, e.g., Scientific Tables, Geigy Pharmaceuticals, Ardsley, New York, 537 (1970). As used herein, “patient” refers to a mammal, including a human.

Unless otherwise stated, structures depicted herein also are meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein also are meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays, or as therapeutic agents.

Chemical structures and nomenclature are derived from ChemDraw, version 11.0.1, Cambridge, MA.

It is noted that the use of the descriptors “first,” “second,” “third,” or the like is used to differentiate separate elements (e.g., solvents, reaction steps, processes, reagents, or the like) and may or may not refer to the relative order or relative chronology of the elements described.

### II. Bifunctional Compounds of the Present Invention

The present invention provides bifunctional compounds that induce the proteolytic degradation of targeted BTK via a ubiquitin proteosome pathway. Certain compounds of the invention also degrade the ubiquitin ligase (e.g., E3 ligase).

A. Bifunctional Compounds

The present invention provides a compound of Formula (A)

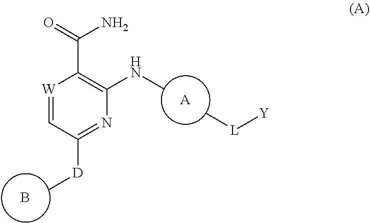

or a pharmaceutically acceptable salt thereof, wherein W is CH or N; D is a bond or —NH—; ring A is phenyl, a 9-10 membered bicyclic aryl, a 5-6 membered partially or fully unsaturated monocyclic heterocycle, or a 9-10 membered bicyclic heteroaryl, wherein the monocyclic heterocycle and bicyclic heteroaryl of ring A each possess 1-3 heteroatoms independently selected from N, O, or S, wherein ring A is optionally and independently substituted with up to 3 substituents selected from halo, —CN, —COOH, NH₂, and optionally substituted C₁₋₆ alkyl; ring B is a phenyl, a 5-6 membered heteroaryl, a 4-6 membered heterocycloalkyl, or a 8-10 membered (e.g., 8-9 membered or 9-10 membered) spiro bicyclic heterocycle, wherein ring B is optionally substituted, and wherein the heteroaryl and heterocycloalkyl of ring B has 1-3 heteroatoms independently selected from N, O, or S; L is —X¹—X²—X³—X⁴—X⁵—; X¹ is a bond, —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro or fused bicyclic heterocycloalkyl having 1-3 heteroatoms independently selected from N, O, or S, or 4-6 membered monocyclic heterocycloalkyl having 1-2 heteroatoms independently selected from N, O, or S, wherein each of the monocyclic and bicyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having 1-2 heteroatoms independently selected from N, O, or S; X³ is a bond, —C₁₋₈ alkyl-, —C≡C—, 4-6 membered cycloalkyl, —N(R)—, —N(R)—C(O)—, —(O—CH₂—CH₂)ₚ—, —(CH₂—CH₂—O)ₚ—, 4-6 membered heterocycloalkyl having 1-2 heteroatoms independently selected from N, O, or S wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, a 5-6 membered saturated, partially unsaturated, or fully unsaturated carbocycle, or a 5-6 membered saturated, partially unsaturated, or fully unsaturated heterocycle having 1-3 heteroatoms independently selected from N, O, or S; X⁵ is a bond, —C₁₋₄ alkyl-, —N(R)—, —O—, —C(O)—, or —C(O)—N(R)—; each R is independently —H or —C₁₋₃ alkyl (e.g., methyl, ethyl, propyl, or iso-propyl); and each of m, n, and p is independently an integer from 1 to 3 (e.g., 1, 2, or 3); and Y is

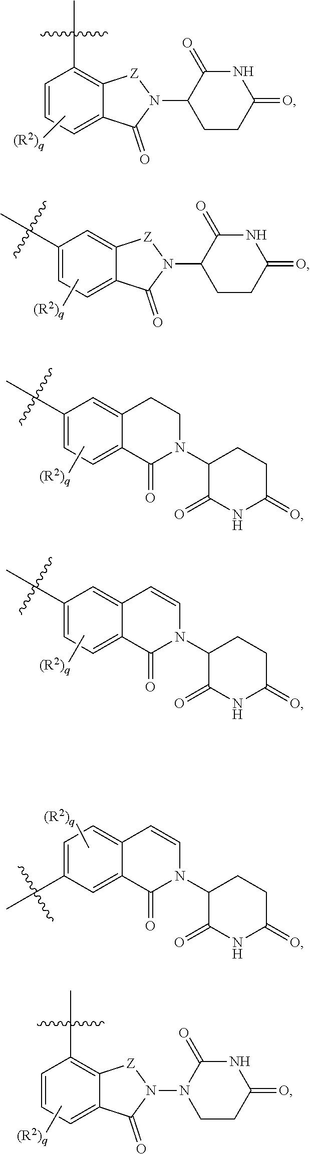

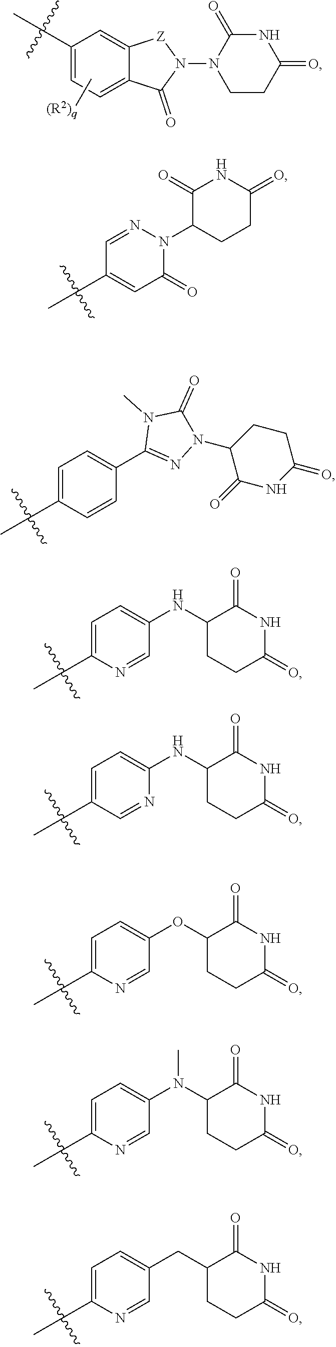

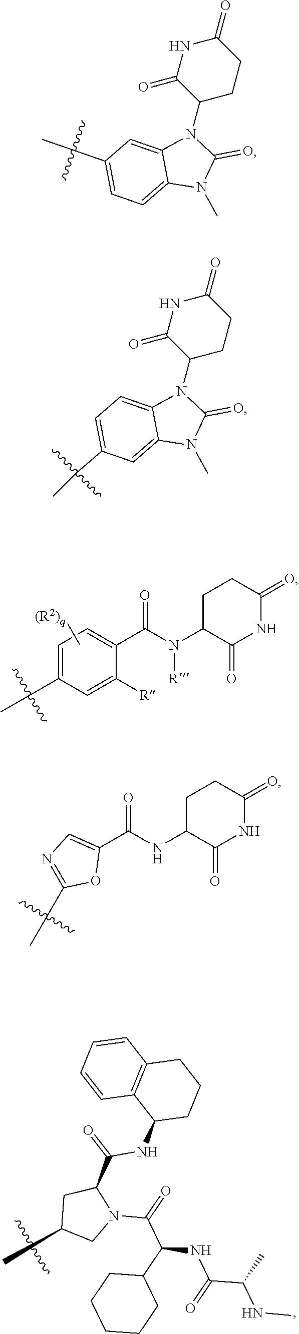

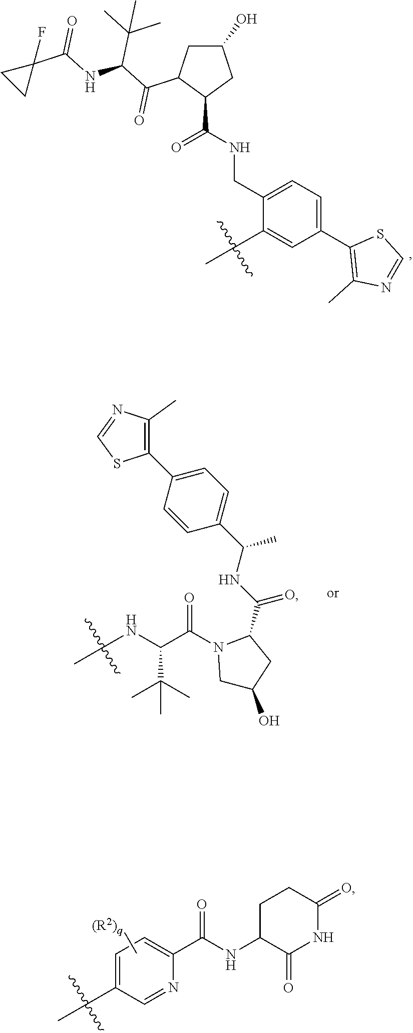

wherein each R² is independently halo, —CN, or —C₁₋₄ alkyl, wherein each C₁₋₄ alkyl is optionally and independently substituted with up to three instances of halo, —CN, —COOH, —COONH₂, —NH₂, or —CF₃; each R″ and R′″ are independently H or, together with the atoms to which they are attached, form a 5-6 membered partially unsaturated or fully unsaturated benzofuzed heterocycle; each Z is —C(R^{A})₂— or —C(O)—; each R^{A} is independently —H or —C₁₋₄ alkyl; and q is 0, 1, or 2.

With the exception of the moieties of group R, all moieties of the linking group L as defined in the compound of Formula (A) are bivalent moieties unless otherwise specified. For example any alkyl (e.g., n-propyl, n-buytl, n-hexyl, and the like), aryl (e.g., phenyl), cycloalkyl (e.g., cyclopropyl, cyclohexyl, and the like), aryl, heteroaryl, heterocylcoalkyl (e.g., piperidine, piperazine, and the like) that is present in L is bivalent unless otherwise specified.

In some embodiments, ring B is an optionally substituted 5-6 membered heterocycloalkyl having 1-2 nitrogen atoms. For example, ring B is piperidine-yl, piperizine-yl, or pyrrolidine-yl, any of which is optionally substituted.

In some embodiments, ring B is an optionally substituted 5-6 membered heteroaryl having 1-2 heteroatoms independently selected from N and S. For example, ring B is pyridine-yl, pyrazine-yl, or pyrimidine, any of which is optionally substituted.

In some embodiments, ring B is

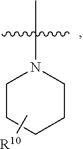

wherein R¹⁰ is halo, —H, —C₁₋₅ alkyl (e.g., —C₁₋₃ alkyl), -3-6 membered cycloalkyl, 5-6 membered heterocycloalkyl, —CN, —OH, —CF₃, —CH₂OH, —CH₂CH₂OH, —C(O)OH,

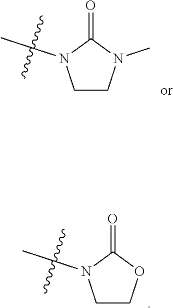

In some embodiments, ring B is

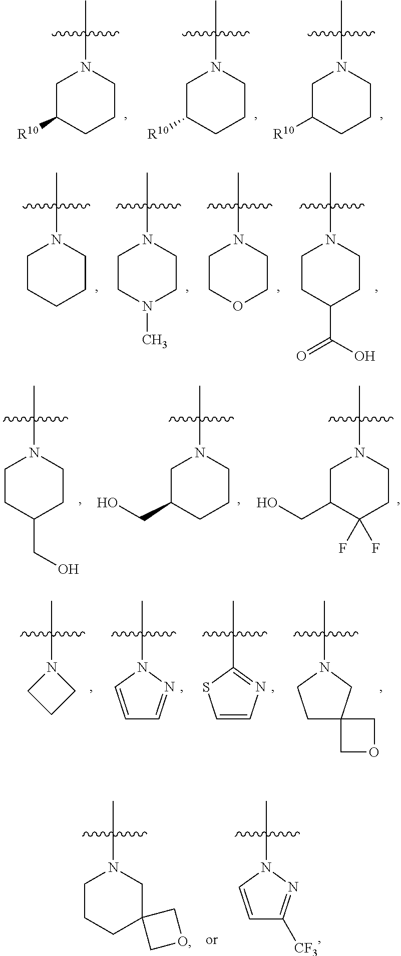

wherein R¹⁰ is

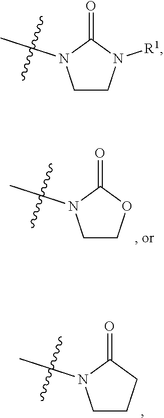

and wherein R¹ is a C₁₋₄ alkyl group. For example, ring B is

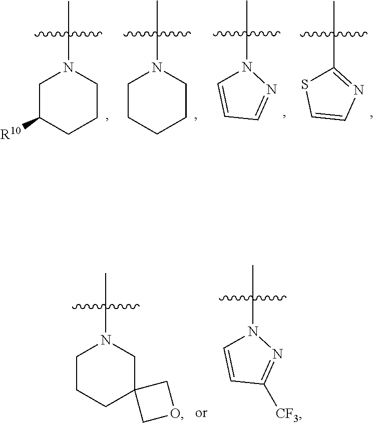

wherein R¹⁰ is

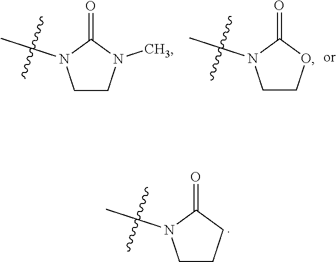 [SMILES: *N1CCN(C)C1=O ; *N1CCOC1=O ; *N1CCCC1=O]

And, in some instances, ring B is

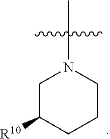

In other instances, R¹⁰ is

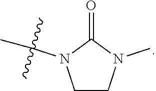 [SMILES: *N1CCN(C)C1=O]

In some embodiments, ring A is

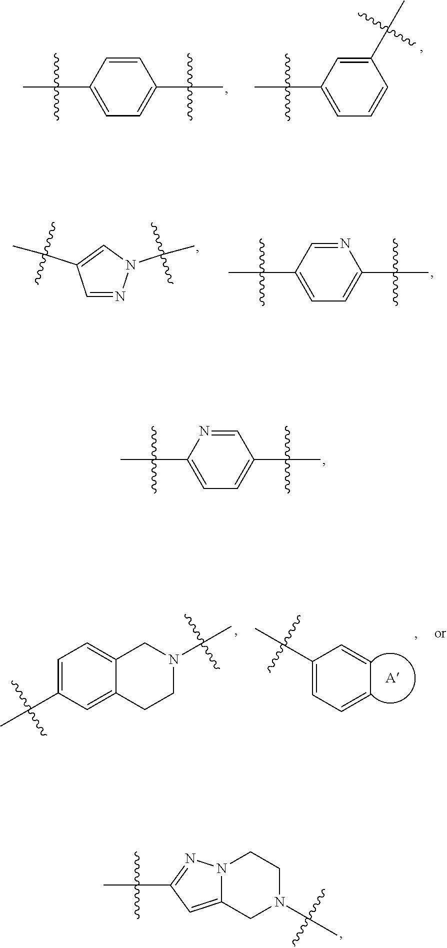

wherein ring A together with the phenyl ring to which it is fused form a 9-10 membered bicyclic aryl or a 9-10 membered bicyclic heteroaryl wherein the bicyclic heteroaryl (i.e., the bicyclic heteroaryl including ring A′) has 1-3 heteroatoms independently selected from N, O, or S. For example, ring A is

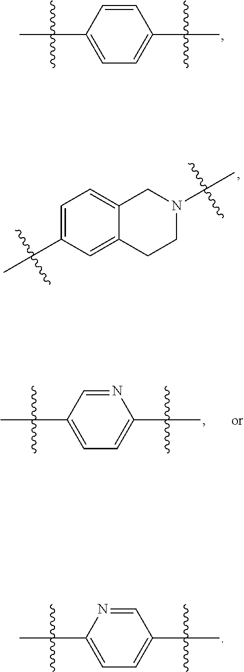 [SMILES: *c1ccc(*)cc1 ; *c1ccc2c(c1)CCN(*)C2 ; *c1ccc(*)nc1 ; *c1ccc(*)nc1]

In some embodiments, at least one of X¹, X², and X⁵ is —N(R)—, —C(O)—N(R)—, or —CH₂—.

In some embodiments, X¹ is —C(O)—N(R)—.

In some embodiments, X² is —(O—CH₂—CH₂)ₙ—, —(CH₂—CH₂—O)ₙ—, or —C₁₋₅ alkyl-.

In some embodiments, X³ is a bond, —C≡C—, —C₁₋₄ alkyl-, or —N(R)—.

In some embodiments, X⁴ is a bond, —CH₂—, or —N(R)—.

In some embodiments, X⁵ is a bond.

In some embodiments, X¹ is —(O—CH₂—CH₂—CH₂)ₘ—, m is 1, and X² is —C(O)—N(R)—.

In some embodiments, X¹ is —CH₂—, —C(O)—,

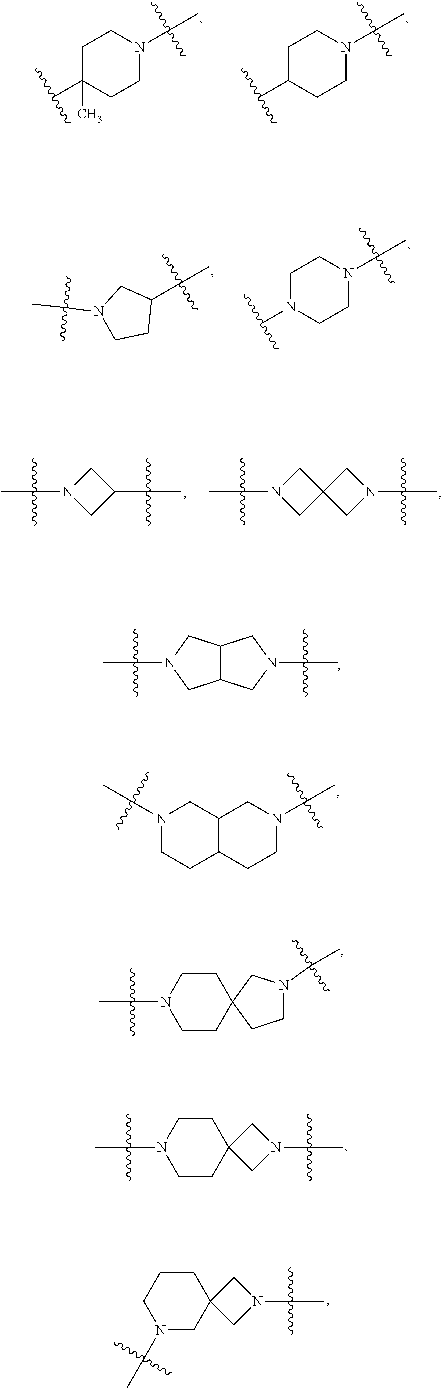

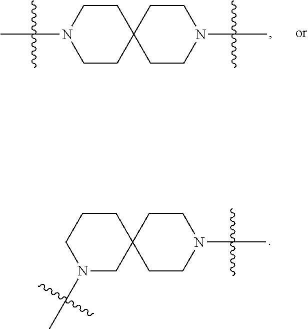 [SMILES: *N1CCC2(CC1)CCN(*)CC2 ; *N1CCC2(CCCN(*)C2)CC1]

In some embodiments, X² is a bond, —C(O)—, —C₁₋₅ alkyl-,

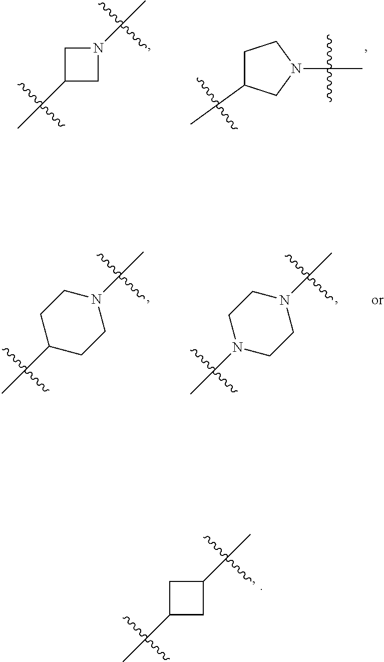 [SMILES: *C1CN(*)C1 ; *C1CCN(*)C1 ; *C1CCN(*)CC1 ; *N1CCN(*)CC1 ; *C1CC(*)C1]

In some embodiments, X³ is bond, —C₁₋₄ alkyl-, 4-6 membered cycloalkyl, or —N(R)—.

In some embodiments, X³ is a bond, —C₁₋₄ alkyl-, —NH—

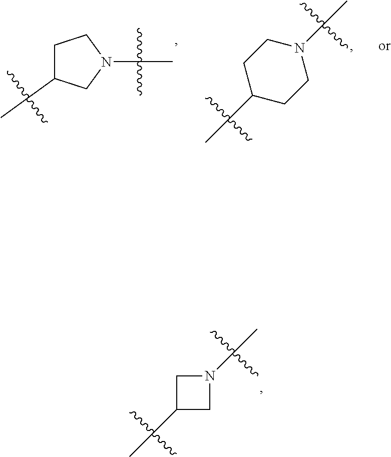 [SMILES: *C1CCN(*)C1 ; *C1CCN(*)CC1 ; *C1CN(*)C1]

### or —C≡C—.

In some embodiments, X⁴ is a bond,

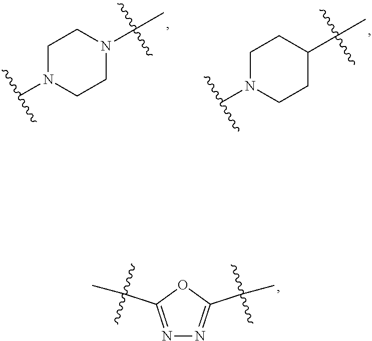 [SMILES: *N1CCN(*)CC1 ; *C1CCN(*)CC1 ; *c1nnc(*)o1]

—C₁₋₄ alkyl-, —CH₂—CH₂—N(R)—, or —N(R)—.

In some embodiments, X⁵ is a bond, —C₁₋₄ alkyl-, —N(R)—, or —C(O)—N(R)—.

In some embodiments, L is

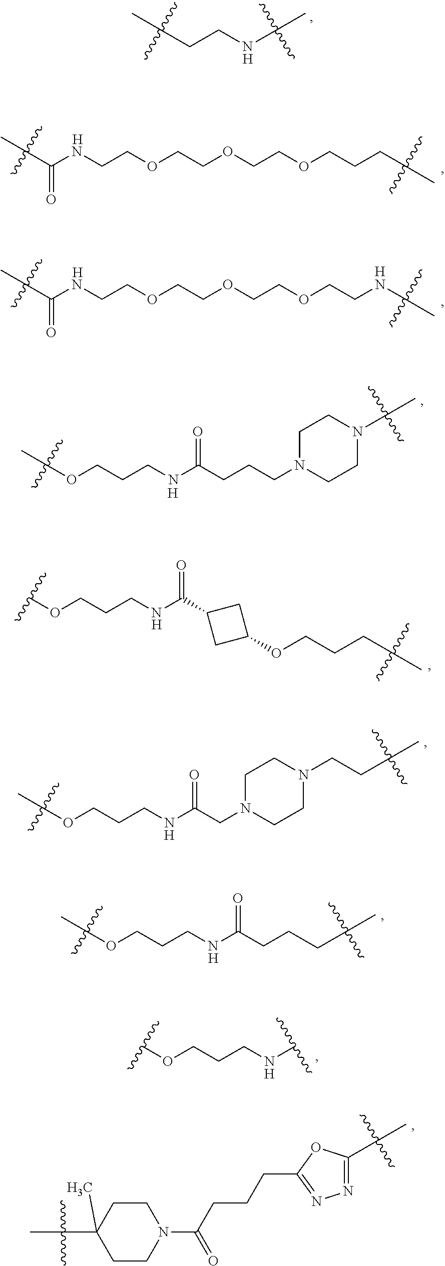

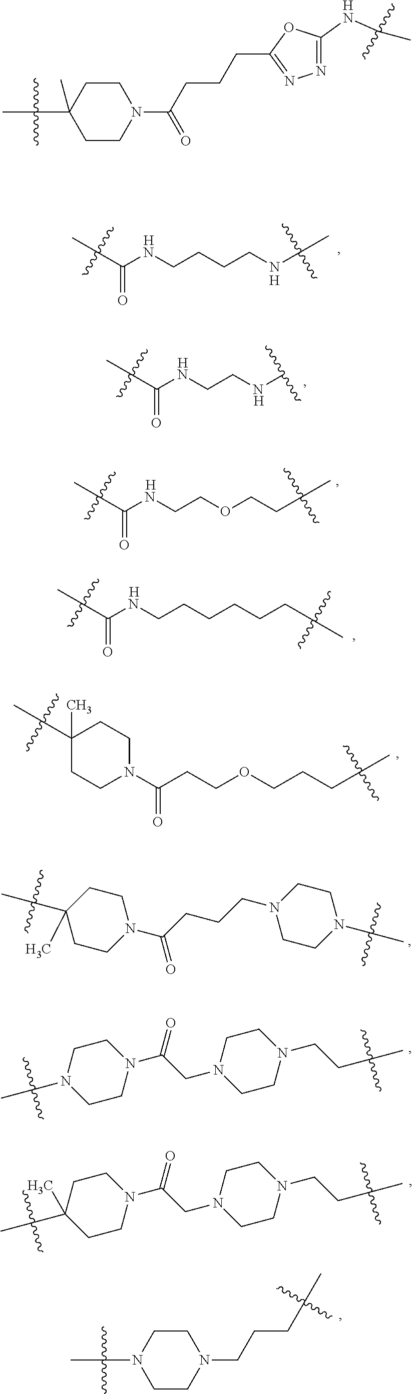

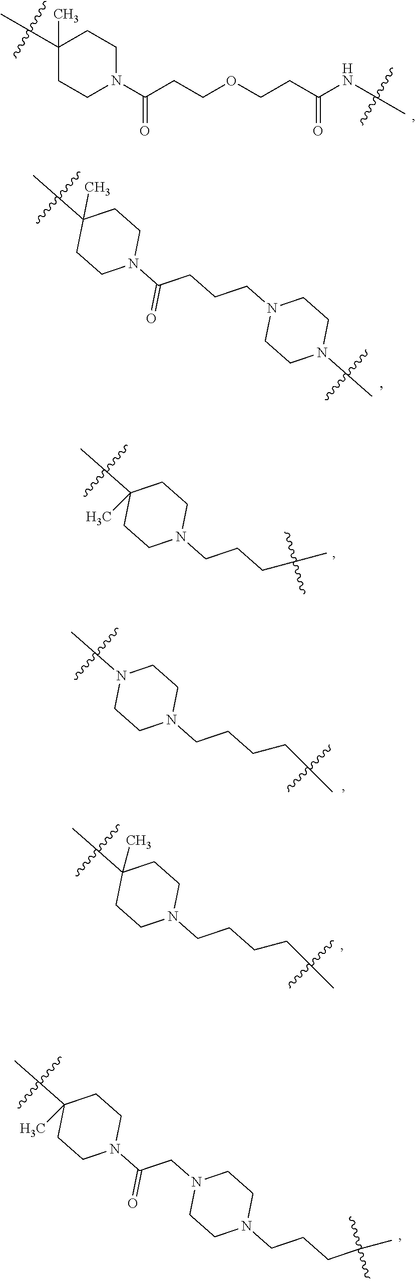

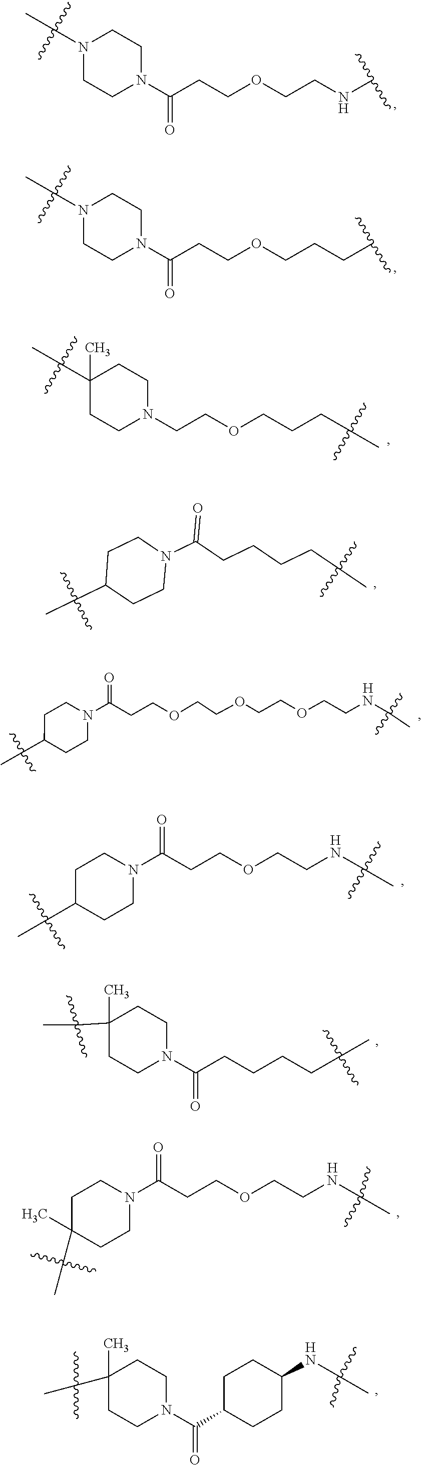

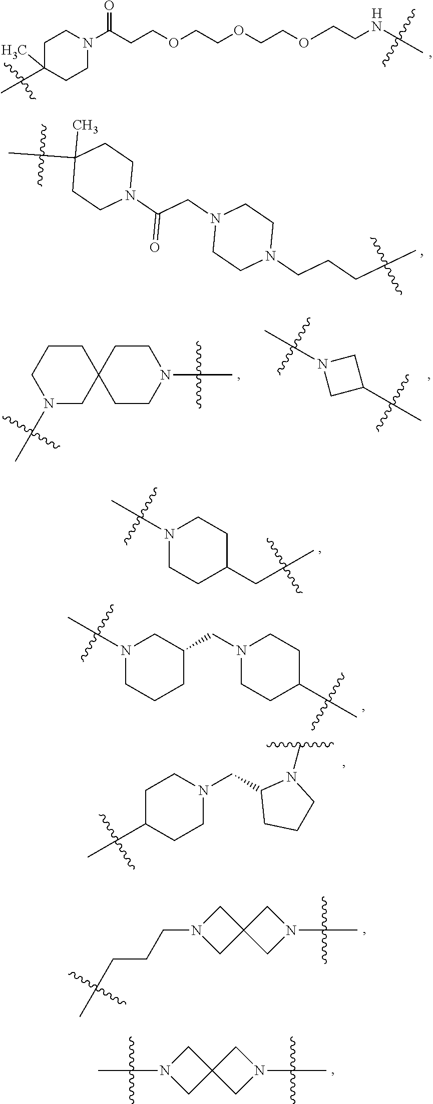

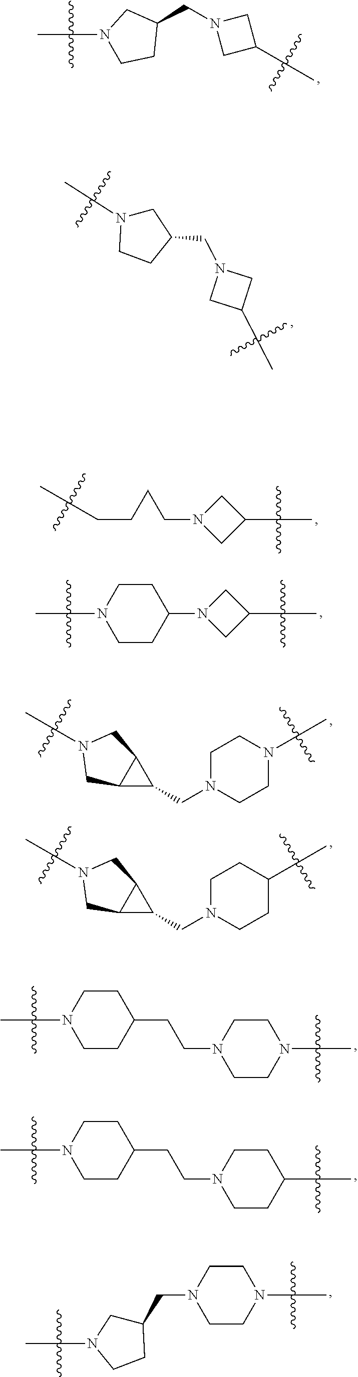

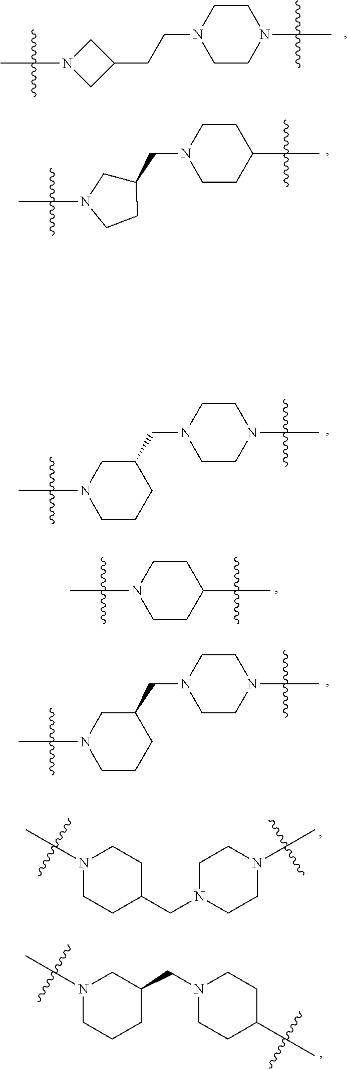

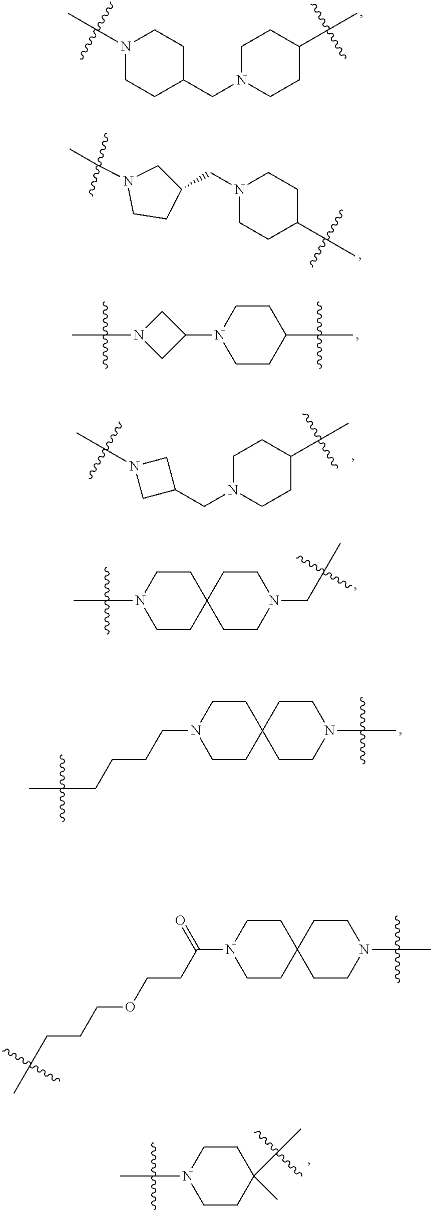

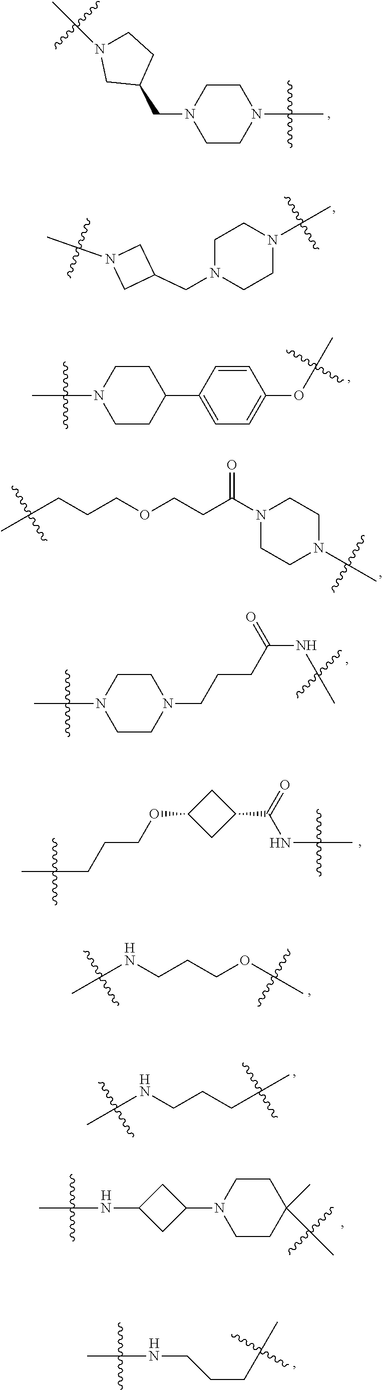

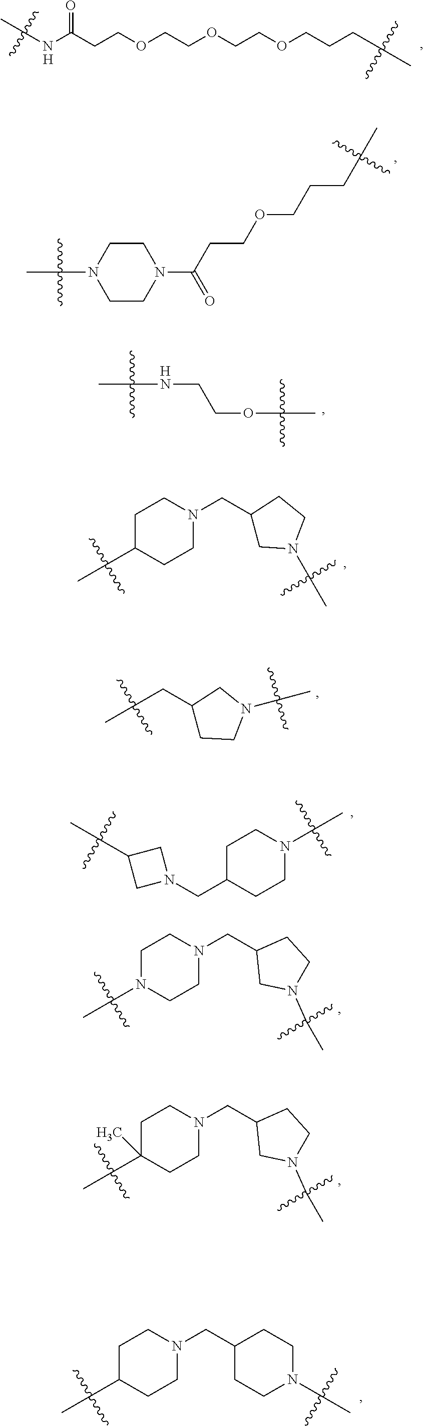

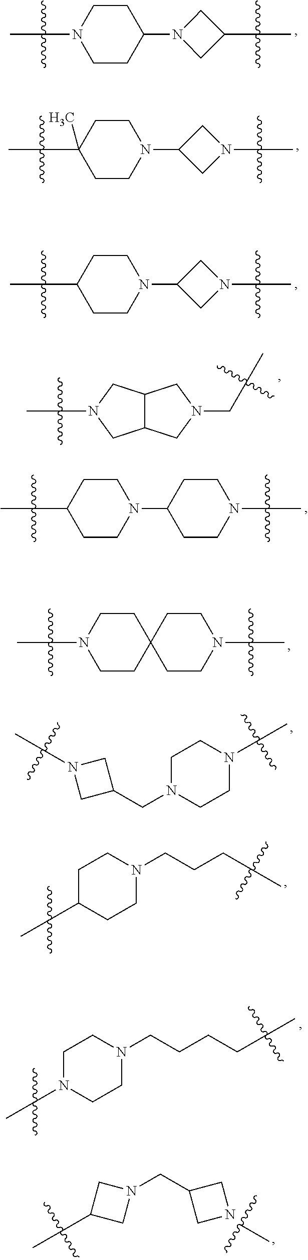

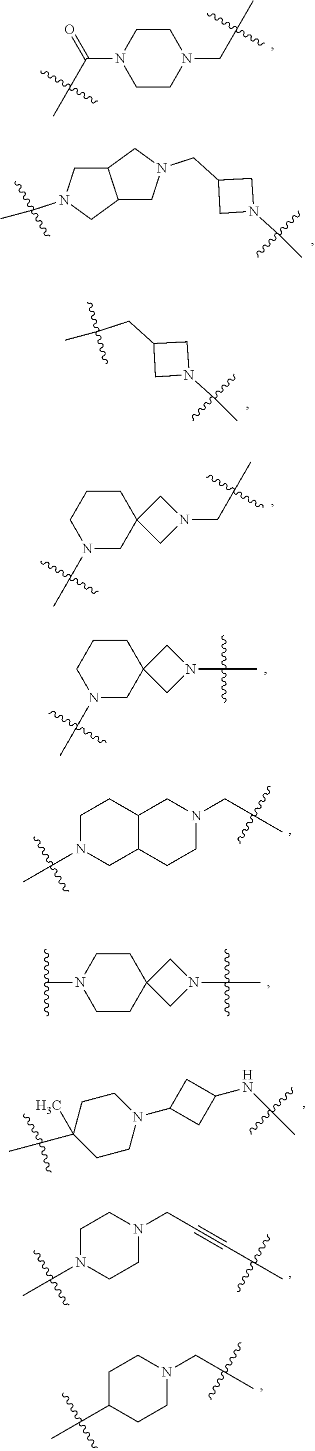

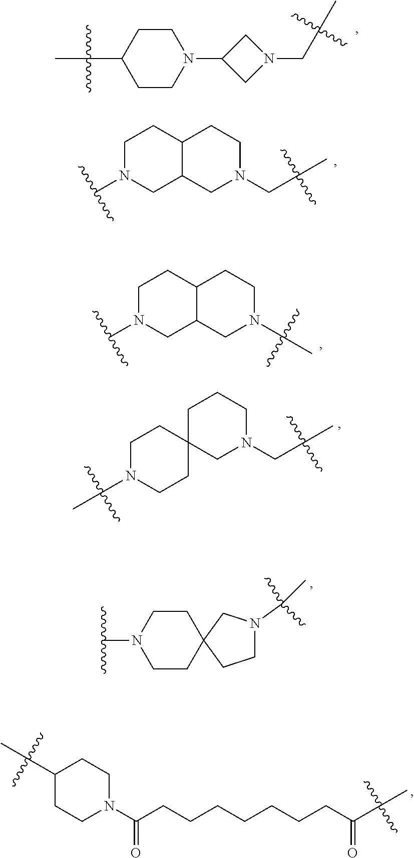

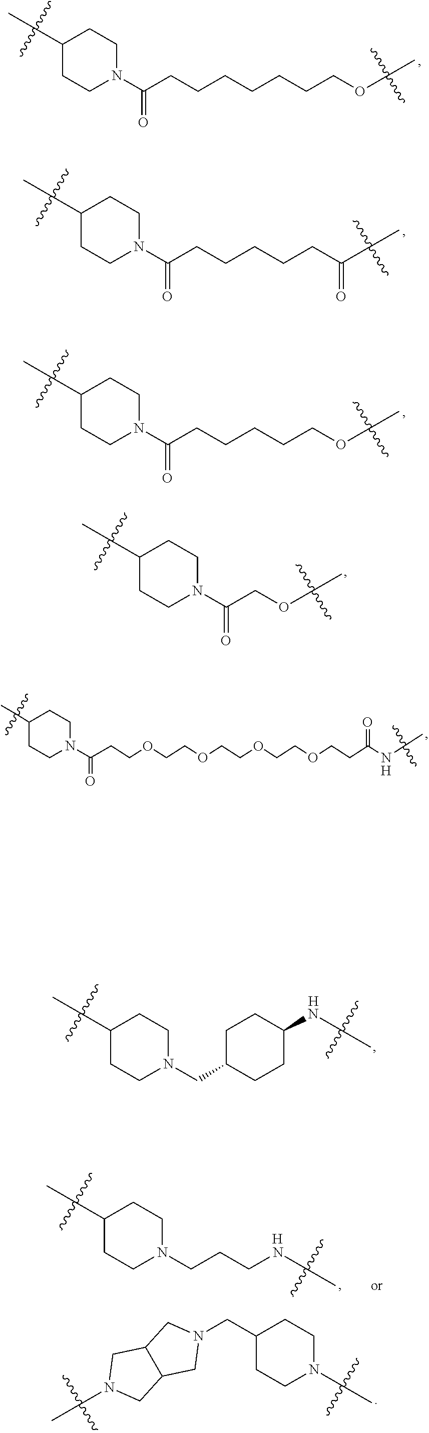

In some embodiments, Y is

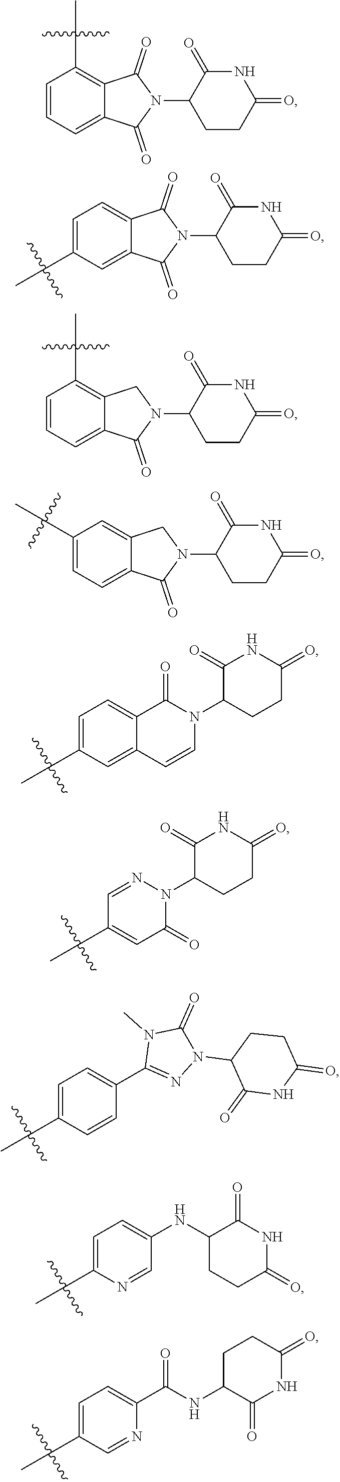

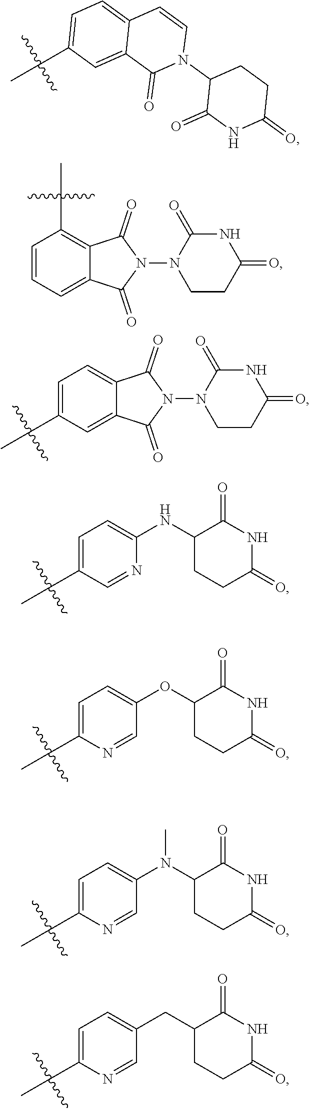

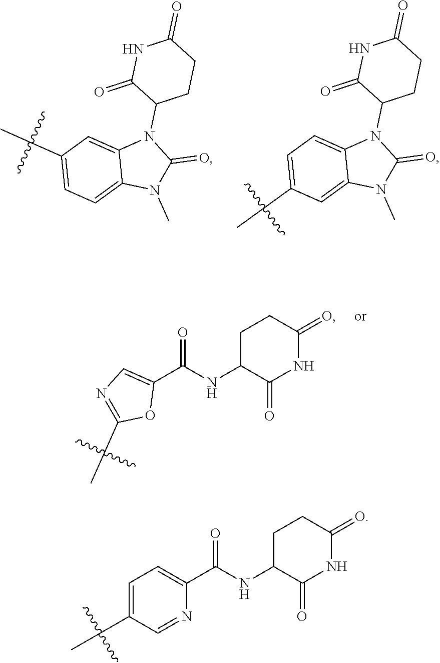

In some embodiments, W is N.

In some embodiments, D is a bond.

The present invention also provides a compound of Formula (B)

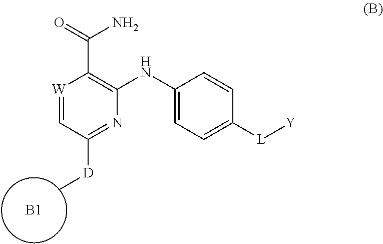

or a pharmaceutically acceptable salt thereof, wherein W is CH or N; D is a bond or —NH—; ring B1 is a 4-6 membered, fully saturated, partially unsaturated, or fully unsaturated monocyclic heterocycle or a 8-10 membered, fully saturated, spiro bicyclic heterocycle, wherein ring B1 has 1-3 heteroatoms independently selected from N, O, or S, and is optionally substituted with 1-3 groups selected from halo, —CH₃, —CF₃, —C(O)OH, —CH₂OH, or a 5 membered heterocycloalkyl optionally substituted with oxo and having 1-2 heteroatoms independently selected from N or 0; L is —X¹—X²—X³—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro or fused bicyclic heterocycloalkyl having 1-3 heteroatoms independently selected from N, O, or S, or 4-6 membered monocyclic heterocycloalkyl having 1-2 heteroatoms independently selected from N, O, or S, wherein each of the monocyclic and bicyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having 1-2 heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having 1-2 heteroatoms independently selected from N, O, or S wherein the heterocycloalkyl is optionally substituted with —CH₃; each R is independently —H or —C₁₋₃ alkyl; each of m, n, and p is independently an integer from 1 to 3; and Y is

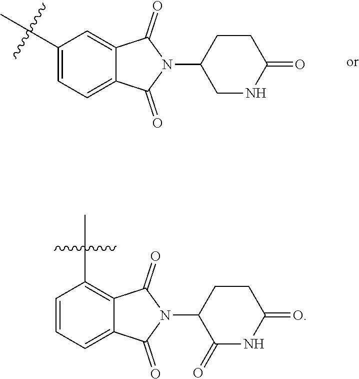 [SMILES: *c1ccc2c(c1)C(=O)N(C1CCC(=O)NC1)C2=O ; *c1cccc2c1C(=O)N(C1CCC(=O)NC1=O)C2=O]

In some embodiments, ring B1 is

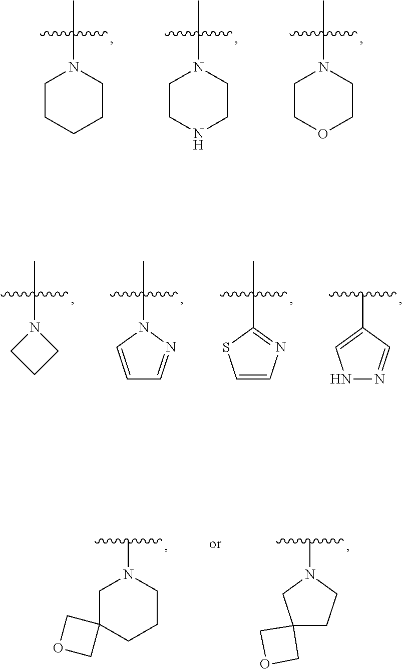

and ring B1 is optionally substituted 1-3 groups selected from —CH₃, —CH₂OH, —CH₂CH₂OH, —C(O)OH, —CF₃, —F,

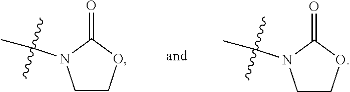 [SMILES: *N1CCOC1=O ; *N1CCOC1=O]

For example, ring B1 is

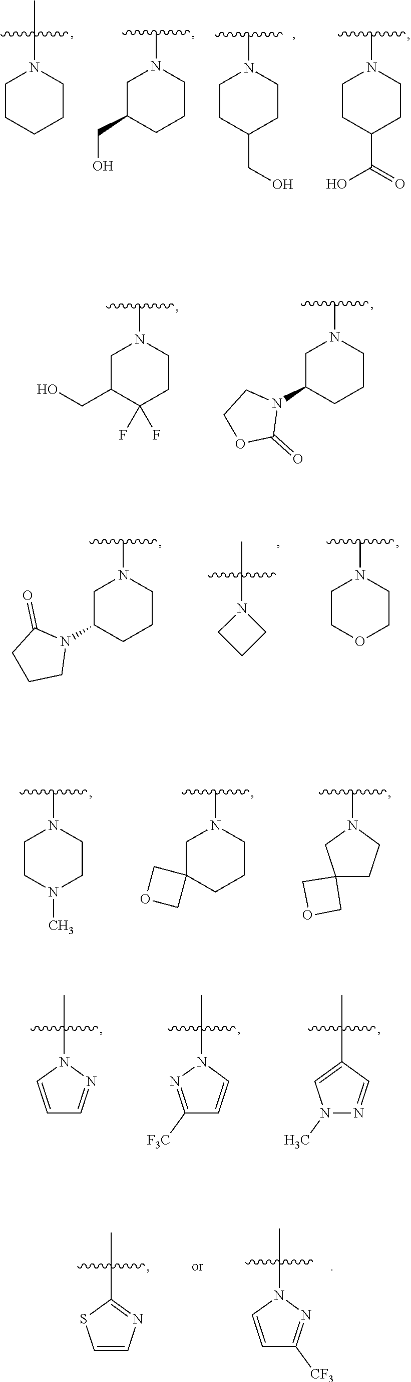

In other examples, ring B1 is

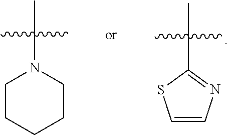 [SMILES: *N1CCCCC1 ; *c1nccs1]

In some embodiments, X¹ is

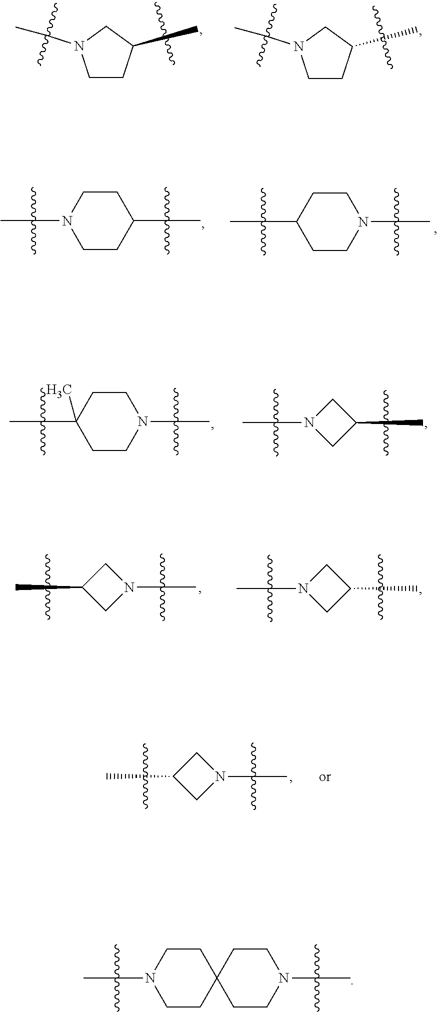

In some embodiments, X² is a bond, —C₁₋₅ alkyl-, 4-6 membered monocyclic cycloalkyl, or 4-6 membered monocyclic heterocycloalkyl having 1-2 heteroatoms independently selected from N, O, or S. For example, X² is a bond, —C₁₋₃ alkyl-, —C(O)—,

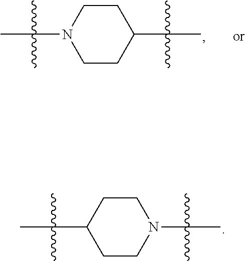 [SMILES: *C1CCN(*)CC1 ; *C1CCN(*)CC1]

In some embodiments, X³ is a bond, —C₁₋₄ alkyl-, —N(R)—, —(O—CH₂—CH₂)ₚ—, —(CH₂—CH₂—O)ₚ—, or a 4-6 membered heterocycloalkyl having 1-2 heteroatoms independently selected from N, O, or S wherein the heterocycloalkyl is optionally substituted with —CH₃. For example, X³ is a bond,

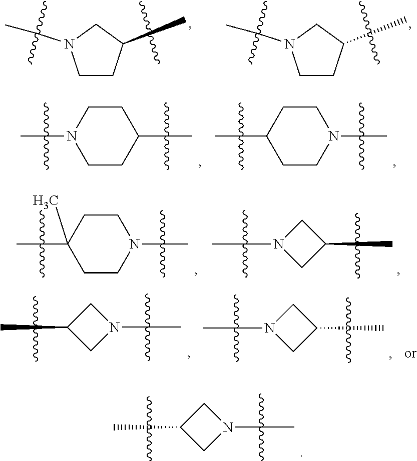

In some embodiments L is

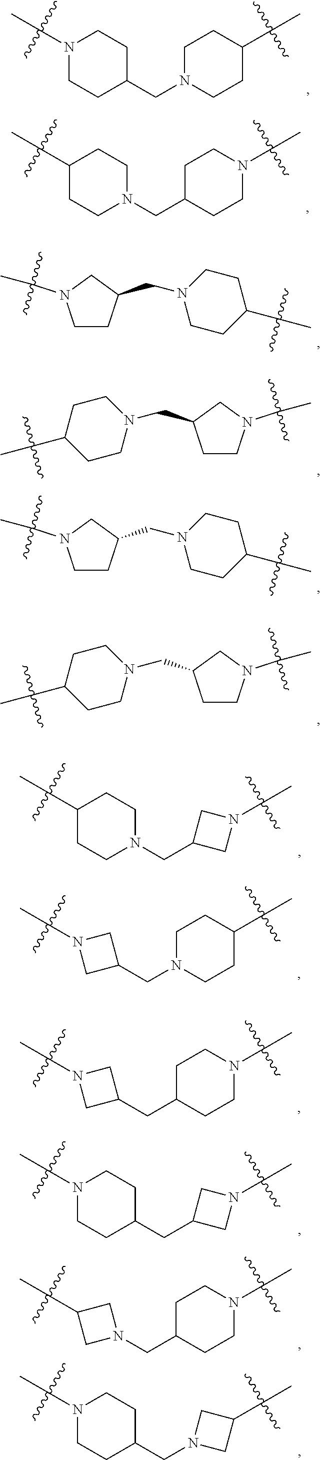

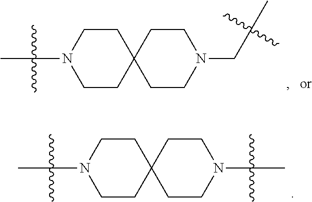 [SMILES: *CN1CCC2(CCN(*)CC2)CC1 ; *N1CCC2(CC1)CCN(*)CC2]

In some embodiments, W is N and D is a bond.

The present invention also provides a compound of Formula (C)

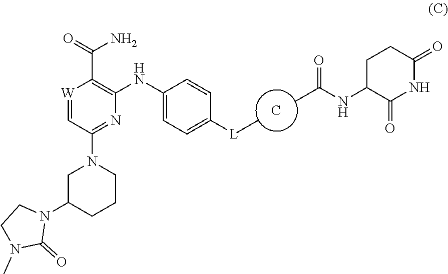

or a pharmaceutically acceptable salt thereof, wherein W is CH or N; ring C is phenyl or a saturated, partially unsaturated, or fully unsaturated 5-6 membered monocyclic heterocycle having 1-2 heteroatoms independently selected from N, O, or S, wherein each of the phenyl and heterocycle of ring C is optionally substituted; L is —X¹—X²—X³—; X¹ is —C(O)—N(R)—, —N(R)—C(O)—, —(O—CH₂—CH₂)ₘ—, —O—(C₆H₄)—, —(O—CH₂—CH₂—CH₂)ₘ—, —C₁₋₅ alkyl-, 7-12 membered spiro bicyclic heterocycloalkyl having 1-3 heteroatoms independently selected from N, O, or S, or 4-6 membered monocyclic heterocycloalkyl having 1-2 heteroatoms independently selected from N, O, or S, wherein each of the bicyclic heterocycloalkyl and the monocyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having 1-2 heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having 1-2 heteroatoms independently selected from N, O, or S wherein the heterocycloalkyl is optionally substituted with —CH₃; each R is independently —H or —C₁₋₃ alkyl; and each of m, n, and p is independently an integer from 1 to 3.

In some embodiments, W is N.

In some embodiments, ring C is

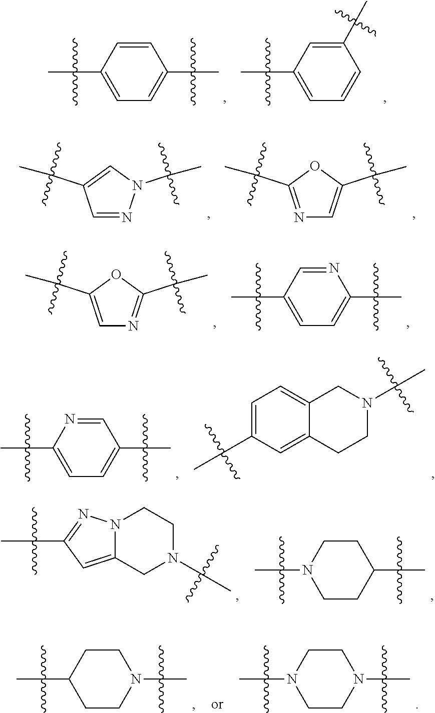

For example, ring C is

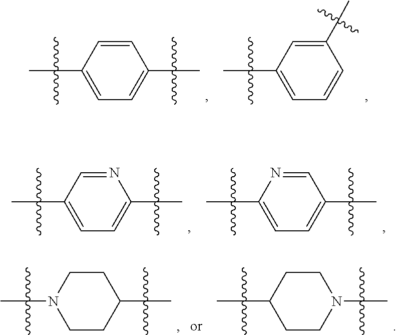

In other examples, ring C is

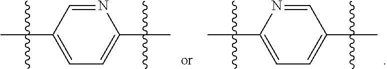 [SMILES: *c1ccc(*)nc1 ; *c1ccc(*)nc1]

In some embodiments, X¹ is a 4-6 membered monocyclic heterocycloalkyl having 1-2 heteroatoms independently selected from N, O, or S. For example, X¹ is

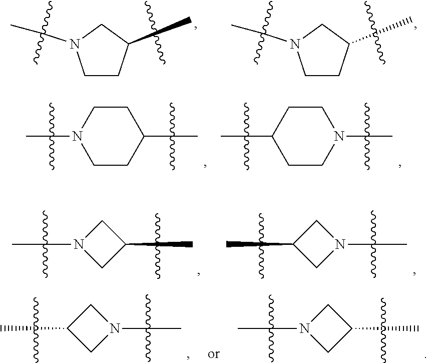

In some examples, X¹ is

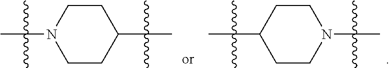 [SMILES: *C1CCN(*)CC1 ; *C1CCN(*)CC1]

In some embodiments, X² is a bond, —C₁₋₅ alky-, 4-6 membered monocyclic cycloalkyl, or 4-6 membered monocyclic heterocycloalkyl having 1-2 heteroatoms independently selected from N, O, or S. For example, X² is a bond or —C₁₋₃ alkyl- (e.g., —CH₂—).

In some embodiments, X³ is a 4-6 membered cycloalkyl, —N(R)—, or a 4-6 membered heterocycloalkyl having 1-2 heteroatoms independently selected from N, O, or S wherein the heterocycloalkyl is optionally substituted with —CH₃. For example, X³ is

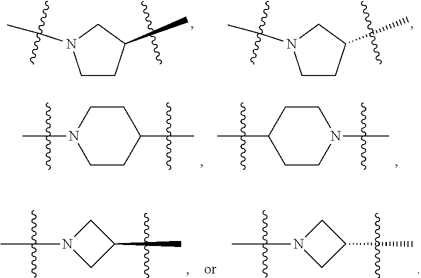

In other examples, X³ is

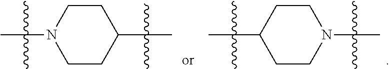 [SMILES: *C1CCN(*)CC1 ; *C1CCN(*)CC1]

In some embodiments, L is

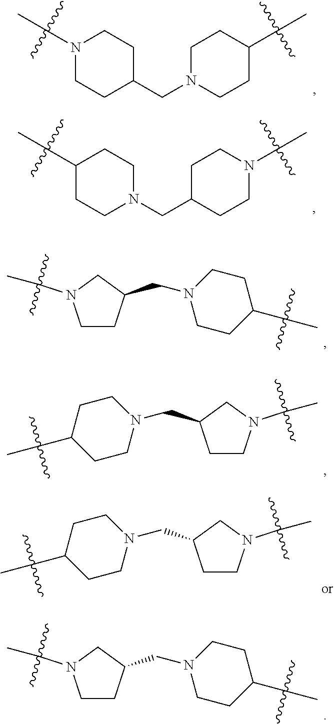 [SMILES: *C1CCN(CC2CCN(*)CC2)CC1 ; *C1CCN(CC2CCN(*)CC2)CC1 ; *C1CCN(C[C@H]2CCN(*)C2)CC1 ; *C1CCN(C[C@@H]2CCN(*)C2)CC1 ; *C1CCN(C[C@H]2CCN(*)C2)CC1 ; *C1CCN(C[C@@H]2CCN(*)C2)CC1]

For example, L is

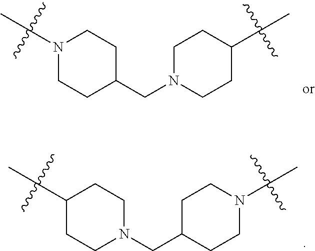 [SMILES: *C1CCN(CC2CCN(*)CC2)CC1 ; *C1CCN(CC2CCN(*)CC2)CC1]

The present invention also provides a compound of Formula (D)

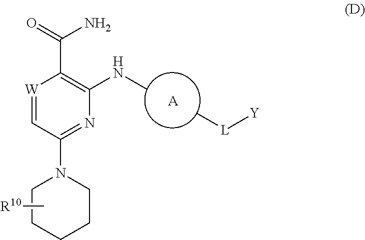

or a pharmaceutically acceptable salt thereof, wherein W is CH or N; ring A is

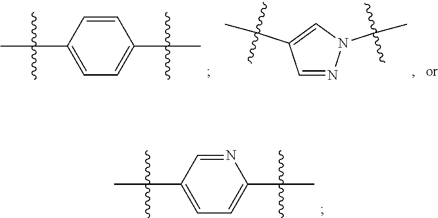 [SMILES: *c1ccc(*)cc1 ; *c1cnn(*)c1 ; *c1ccc(*)nc1]

L is —X¹—X²—X³—; X¹ is —C₁₋₅ alkyl- or 4-6 membered monocyclic heterocycloalkyl having 1-2 heteroatoms independently selected from N, O, or S wherein the monocyclic heterocycloalkyl of X¹ is optionally substituted with —CH₃; X² is a bond, —C₁₋₅ alkyl-, or 4-6 membered monocyclic heterocycloalkyl having 1-2 heteroatoms independently selected from N, O, or S wherein the monocyclic heterocycloalkyl of X¹ is optionally substituted with —CH₃; X³ is a bond, —C₁₋₄ alkyl-, 4-6 membered monocyclic cycloalkyl, or 4-6 membered heterocycloalkyl having 1-2 heteroatoms independently selected from N, O, or S wherein the heterocycloalkyl is optionally substituted with —CH₃; Y is

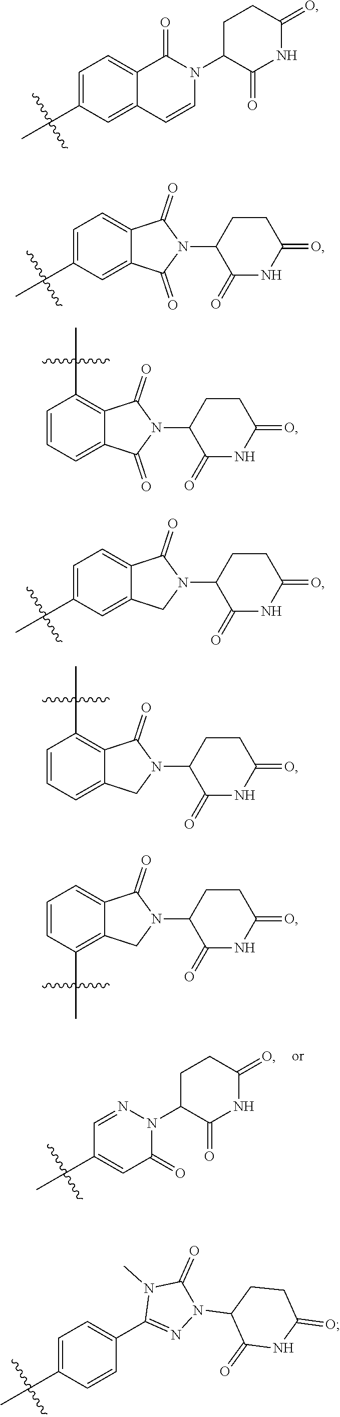

and R¹⁰ is halo, —H, —C₁₋₅ alkyl, -3-6 membered cycloalkyl, 5-6 membered heterocycloalkyl, —CN, —OH, —CF₃, —CH₂OH, —CH₂CH₂OH, —C(O)OH,

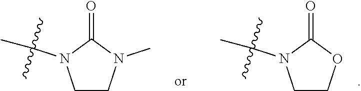

In some embodiments, ring A is

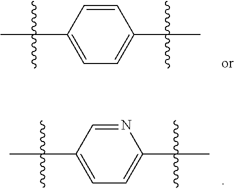 [SMILES: *c1ccc(*)cc1 ; *c1ccc(*)nc1]

In some embodiments, X¹ is a 4-6 membered monocyclic heterocycloalkyl having 1-2 heteroatoms independently selected from N, O, or S wherein the monocyclic heterocycloalkyl of X¹ is optionally substituted with —CH₃. For example, X¹ is

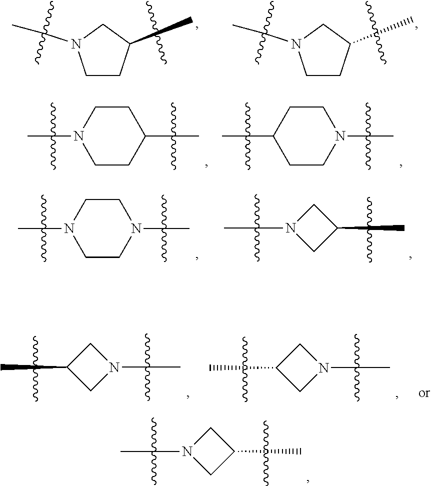

In some embodiments, X² is a bond, —C₁₋₅ alkyl-, 4-6 membered monocyclic cycloalkyl, or 4-6 membered monocyclic heterocycloalkyl having 1-2 heteroatoms independently selected from N, O, or S. For example, X² is a bond or —C₁₋₄ alkyl-.

In some embodiments, X³ is a bond, a 4-6 membered monocyclic cycloalkyl, or 4-6 membered monocyclic heterocycloalkyl having 1-2 heteroatoms independently selected from N, O, or S. For example, X³ is

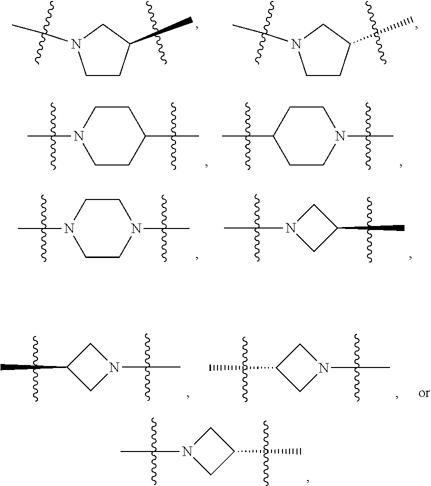

In some embodiments, L is

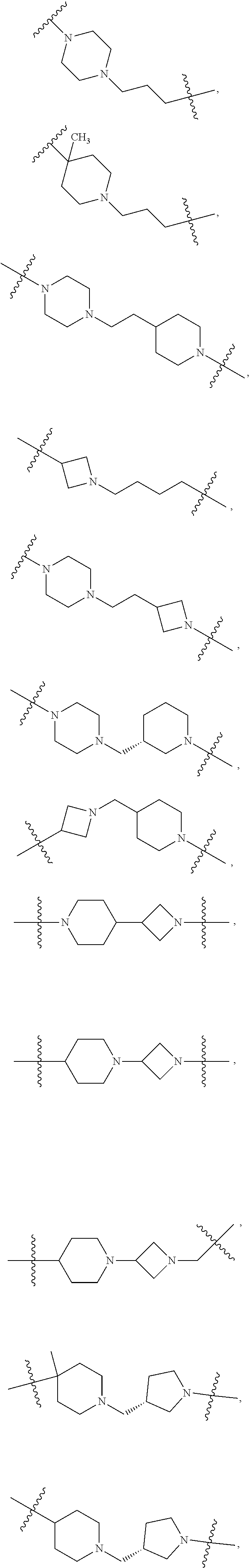

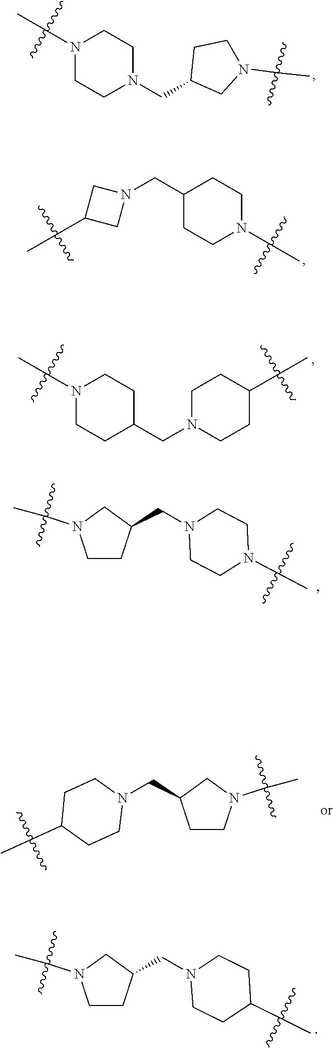 [SMILES: *N1CCN(C[C@@H]2CCN(*)C2)CC1 ; *C1CN(CC2CCN(*)CC2)C1 ; *C1CCN(CC2CCN(*)CC2)CC1 ; *N1CCN(C[C@H]2CCN(*)C2)CC1 ; *C1CCN(C[C@@H]2CCN(*)C2)CC1 ; *C1CCN(C[C@@H]2CCN(*)C2)CC1]

In some embodiments, R¹⁰ is halo, —H, —C₁₋₅ alkyl (e.g., —C₁₋₃ alkyl), -3-6 membered cycloalkyl, 5-6 membered heterocycloalkyl, —CN, —OH, —CF₃, —CH₂OH, —C(O)OH, or —CH₂CH₂OH. For instance, R¹⁰ is halo, —H, C₁₋₃ alkyl, CF₃, —CH₂OH, —C(O)OH, or —CH₂CH₂OH. In other instances, R¹⁰ is or

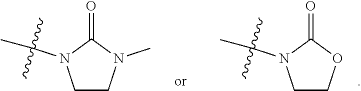

In some embodiments, R¹⁰ is

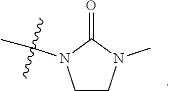 [SMILES: *N1CCN(C)C1=O]

In some embodiments, R¹⁰ is

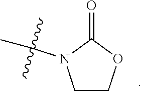 [SMILES: *N1CCOC1=O]

In some embodiments, the compound of Formula (D) is a compound of (D-1)

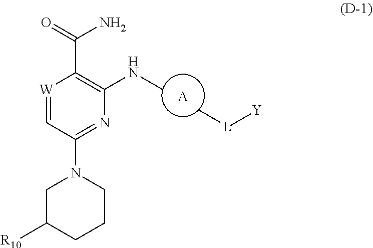

or a pharmaceutically acceptable salt thereof, wherein W is CH or N; ring A is

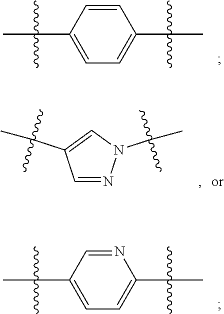 [SMILES: *c1ccc(*)cc1 ; *c1cnn(*)c1 ; *c1ccc(*)nc1]

L is —X¹—X²—X³—; X¹ is —C₁₋₅ alkyl- or 4-6 membered monocyclic heterocycloalkyl having 1-2 heteroatoms independently selected from N, O, or S wherein the monocyclic heterocycloalkyl of X¹ is optionally substituted with —CH₃; X² is a bond, —C₁₋₅ alkyl-, or 4-6 membered monocyclic heterocycloalkyl having 1-2 heteroatoms independently selected from N, O, or S wherein the monocyclic heterocycloalkyl of X¹ is optionally substituted with —CH₃; X³ is a bond, —C₁₋₄ alkyl-, 4-6 membered monocyclic cycloalkyl, or 4-6 membered heterocycloalkyl having 1-2 heteroatoms independently selected from N, O, or S wherein the heterocycloalkyl is optionally substituted with —CH₃; Y is

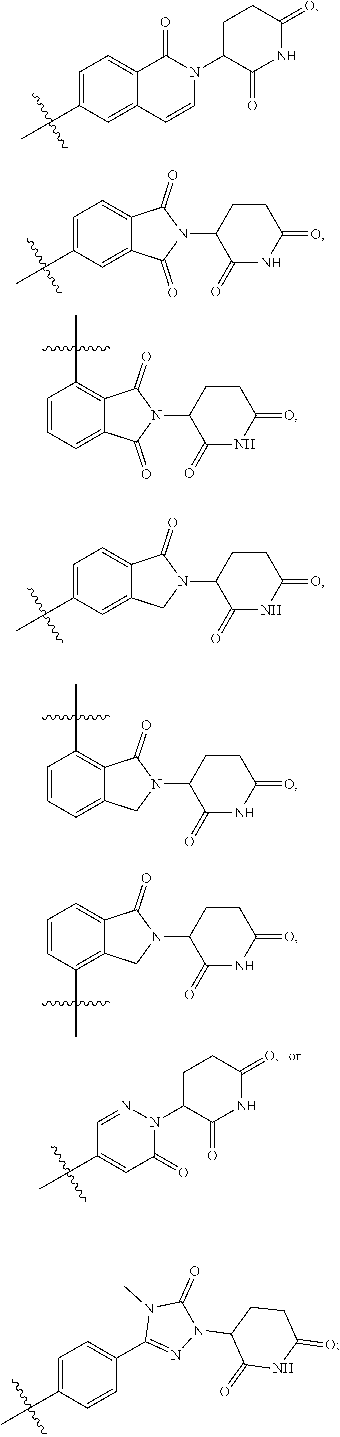

### and R¹⁰ is

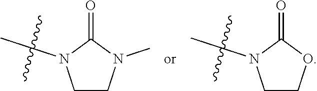

In some embodiments, ring A is

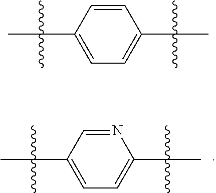 [SMILES: *c1ccc(*)cc1 ; *c1ccc(*)nc1]

In some embodiments, X¹ is a 4-6 membered monocyclic heterocycloalkyl having 1-2 heteroatoms independently selected from N, O, or S wherein the monocyclic heterocycloalkyl of X¹ is optionally substituted with —CH₃. For example, X¹ is

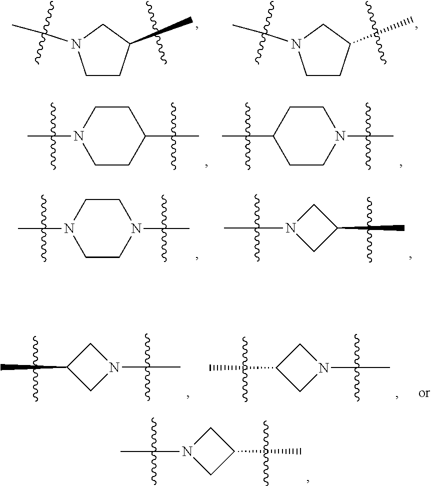

In some embodiments, X² is a bond, —C₁₋₅ alkyl-, 4-6 membered monocyclic cycloalkyl, or 4-6 membered monocyclic heterocycloalkyl having 1-2 heteroatoms independently selected from N, O, or S. For example, X² is a bond or —C₁₋₄ alkyl-.

In some embodiments, X³ is a bond, a 4-6 membered monocyclic cycloalkyl, or 4-6 membered monocyclic heterocycloalkyl having 1-2 heteroatoms independently selected from N, O, or S. For example, X³ is

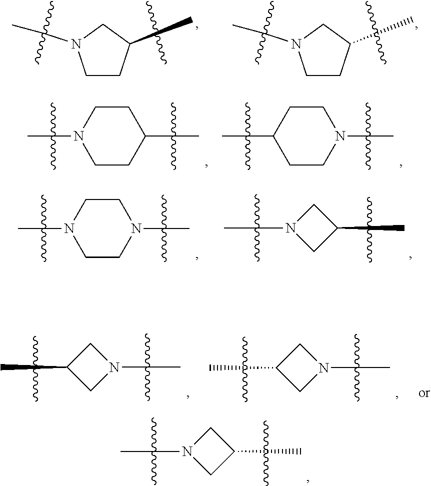

In some embodiments, L is

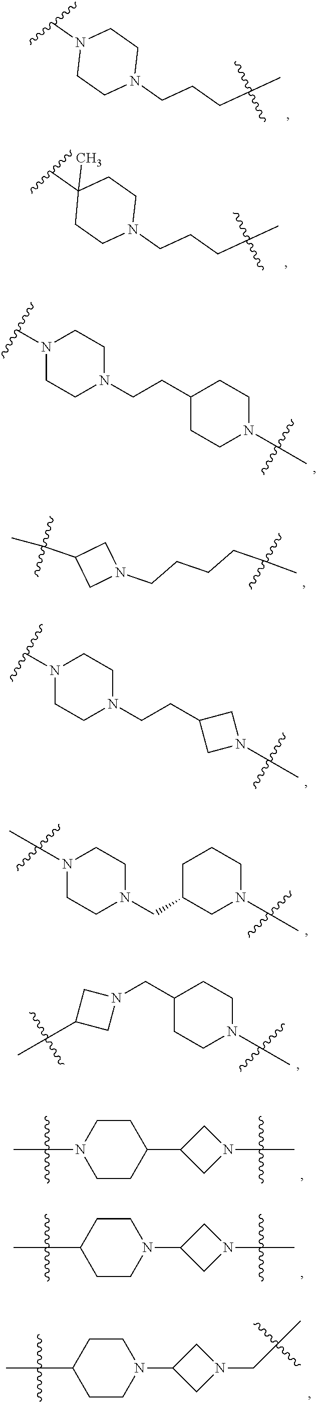

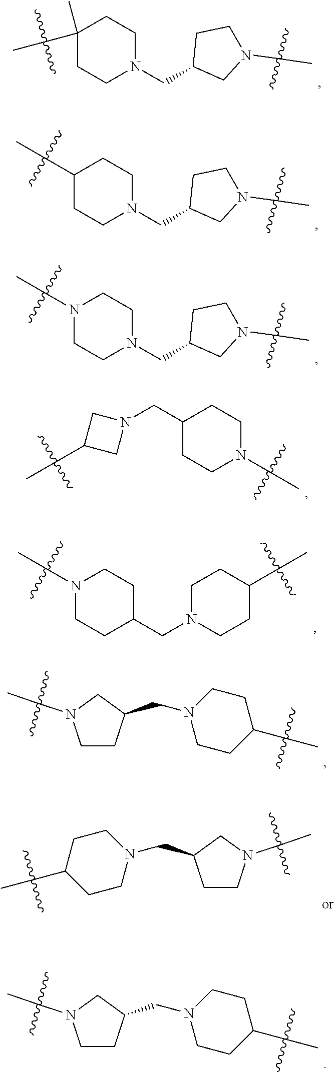

In some embodiments, R¹⁰ is

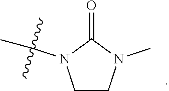 [SMILES: *N1CCN(C)C1=O]

In some embodiments, R¹⁰ is

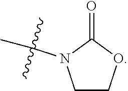 [SMILES: *N1CCOC1=O]

In some embodiments, the compound of Formula (D) or the compound of Formula (D-1) is a compound of Formula (D-2):

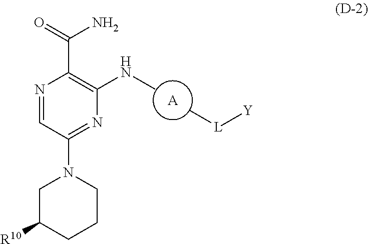

or a pharmaceutically acceptable salt thereof, wherein the terms ring A, L, Y, and R¹⁰ are as defined in the compound of Formula (A), the compound of Formula (D), and the compound of Formula (D-1).

In some embodiments, ring A is

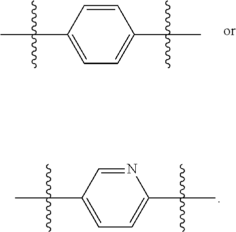 [SMILES: *c1ccc(*)cc1 ; *c1ccc(*)nc1]

In some embodiments, X¹ is a 4-6 membered monocyclic heterocycloalkyl having 1-2 heteroatoms independently selected from N, O, or S wherein the monocyclic heterocycloalkyl of X¹ is optionally substituted with —CH₃. For example, X¹ is

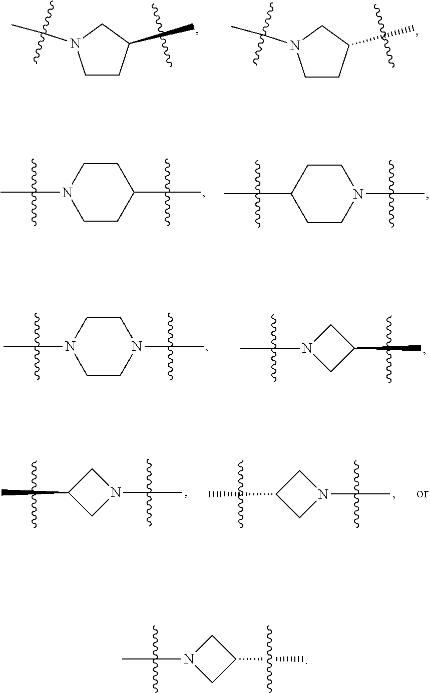

In some embodiments, X² is a bond, —C₁₋₅ alkyl-, 4-6 membered monocyclic cycloalkyl, or 4-6 membered monocyclic heterocycloalkyl having 1-2 heteroatoms independently selected from N, O, or S. For example, X² is a bond or —C₁₋₄ alkyl-.

In some embodiments, X³ is a bond, a 4-6 membered monocyclic cycloalkyl, or 4-6 membered monocyclic heterocycloalkyl having 1-2 heteroatoms independently selected from N, O, or S. For example, X³ is

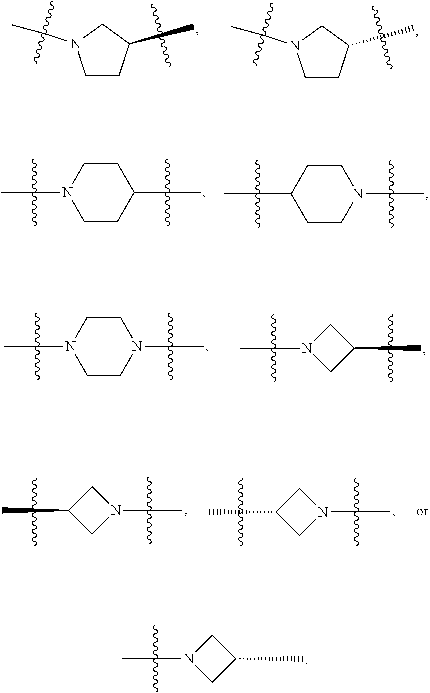

In some embodiments, L is

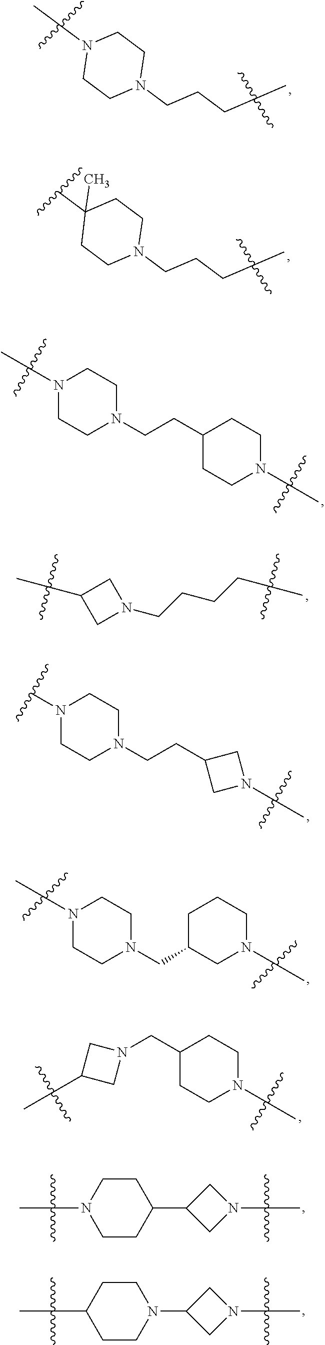

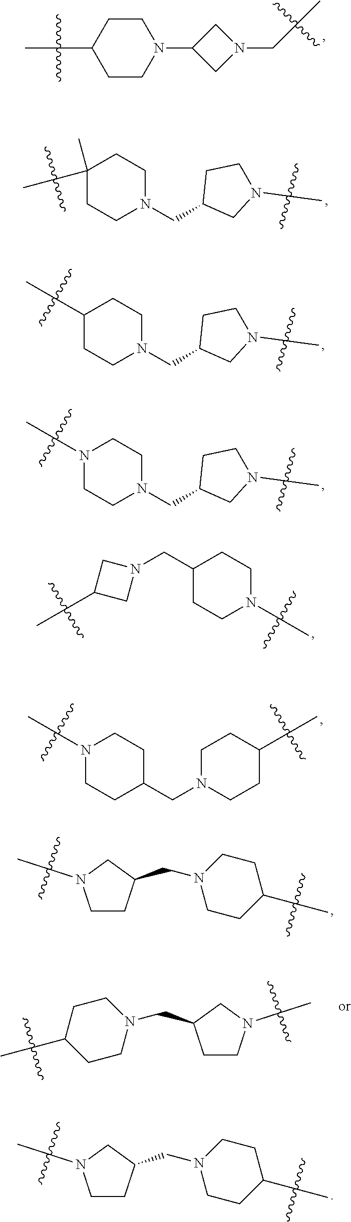

In some embodiments, R¹⁰ is

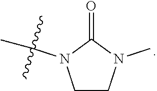 [SMILES: *N1CCN(C)C1=O]

In some embodiments, R¹⁰ is

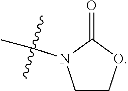 [SMILES: *N1CCOC1=O]

The present invention also provides a compound of Formula (E)

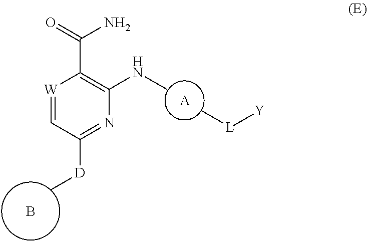

or a pharmaceutically acceptable salt thereof, wherein D is a bond or —NH—; W is N or CH; ring A is phenyl, a 9-10 membered bicyclic aryl, a 5-6 membered partially or fully unsaturated monocyclic heterocycle, or a 9-10 membered bicyclic heteroaryl, wherein the monocyclic heterocycle and bicyclic heteroaryl of ring A each possess 1-3 heteroatoms independently selected from N, O, or S; ring B is an optionally substituted 5-6 membered saturated, partially unsaturated, or fully unsaturated monocyclic heterocycle, or an optionally substituted 8-10 membered (e.g., 8-9 membered or 9-10 membered) spiro bicyclic heterocycle, wherein ring B has 1-3 heteroatoms independently selected from N, O, or S; L is —X¹—X²—X³—X⁴—X⁵—; X¹ is a bond, —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having 1-3 heteroatoms independently selected from N, O, or S, or 4-6 membered monocyclic heterocycloalkyl having 1-2 heteroatoms independently selected from N, O, or S, wherein each of the monocyclic and bicyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having 1-2 heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having 1-2 heteroatoms independently selected from N, O, or S wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, a 5-6 membered saturated, partially unsaturated, or fully unsaturated carbocycle, or a 5-6 membered saturated, partially unsaturated, or fully unsaturated heterocycle having 1-3 heteroatoms independently selected from N, O, or S; X⁵ is a bond, —N(R)—, or —C(O)—N(R)—; each R is independently —H or —C₁₋₃ alkyl; each of m, n, and p is independently an integer from 1 to 3; and Y is

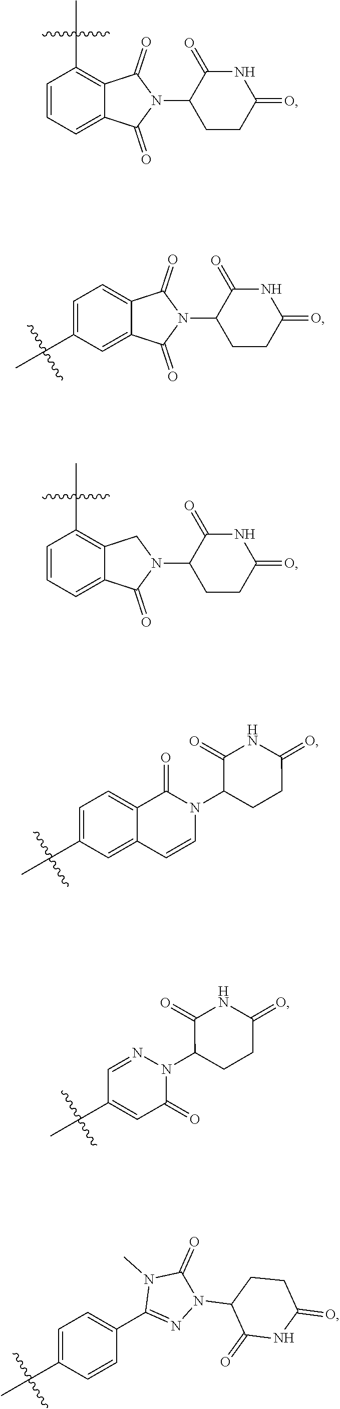 [SMILES: *c1cccc2c1C(=O)N(C1CCC(=O)NC1=O)C2=O ; *c1ccc2c(c1)C(=O)N(C1CCC(=O)NC1=O)C2=O ; *c1cccc2c1CN(C1CCC(=O)NC1=O)C2=O ; *c1ccc2c(=O)n(C3CCC(=O)NC3=O)ccc2c1 ; *c1cnn(C2CCC(=O)NC2=O)c(=O)c1 ; *c1ccc(-c2nn(C3CCC(=O)NC3=O)c(=O)n2C)cc1]

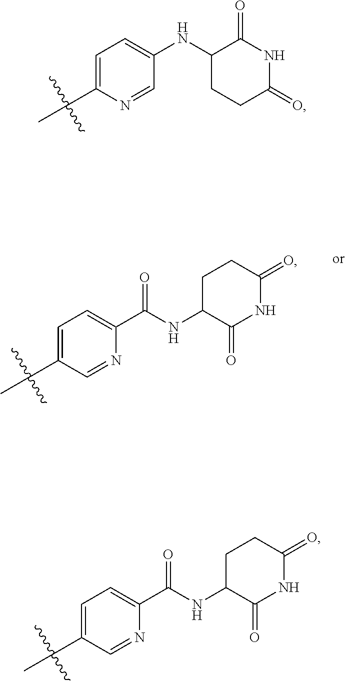 [SMILES: *c1ccc(NC2CCC(=O)NC2=O)cn1 ; *c1ccc(C(=O)NC2CCC(=O)NC2=O)nc1 ; *c1ccc(C(=O)NC2CCC(=O)NC2=O)nc1]

wherein at least one of X¹, X², X³, X⁴, and X⁵ has a nitrogen atom, and Y is directly bonded to L at a nitrogen atom of X¹, X², X³, X⁴, or X⁵.

In some embodiments, ring B is

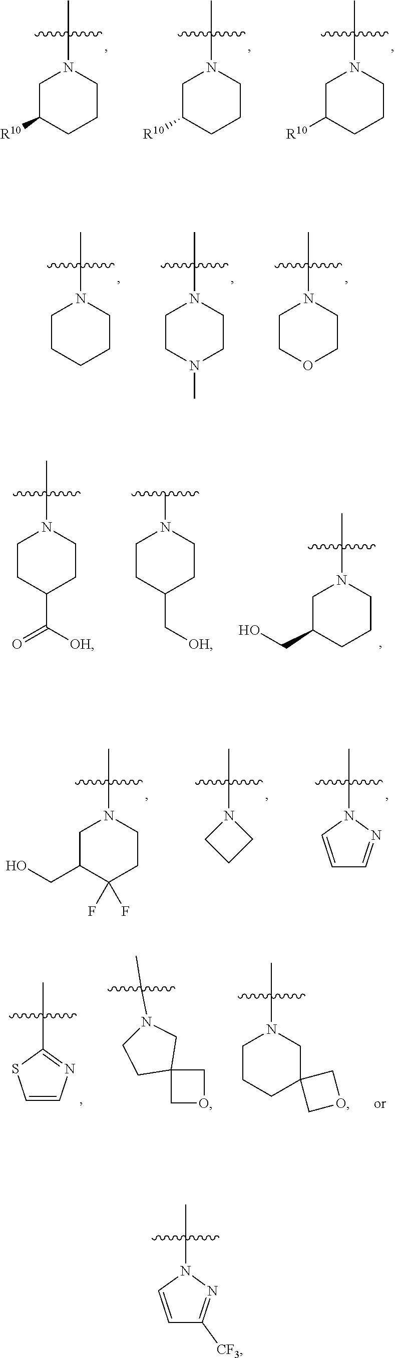

wherein R¹⁰ is

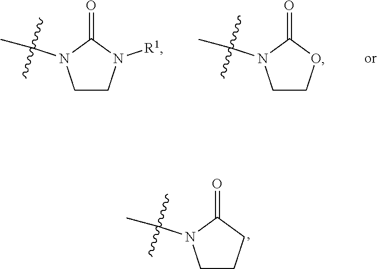

and wherein R¹ is a C₁₋₄ alkyl group. For example, ring B is

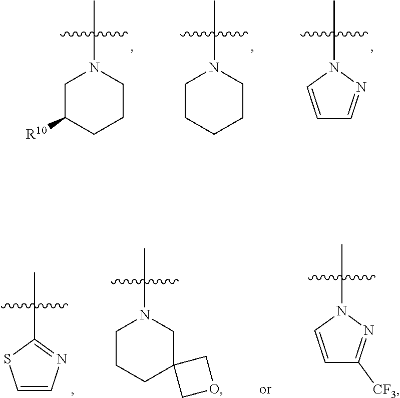

wherein R¹⁰ is

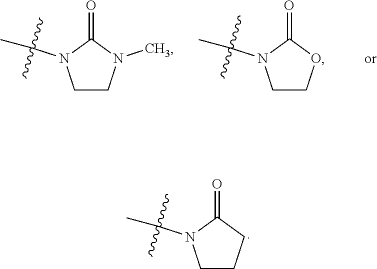 [SMILES: *N1CCN(C)C1=O ; *N1CCOC1=O ; *N1CCCC1=O]

In other examples, ring B is

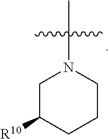

In some embodiments, R¹⁰ is

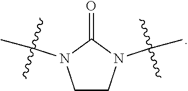 [SMILES: *N1CCN(*)C1=O]

In some embodiments, ring A is

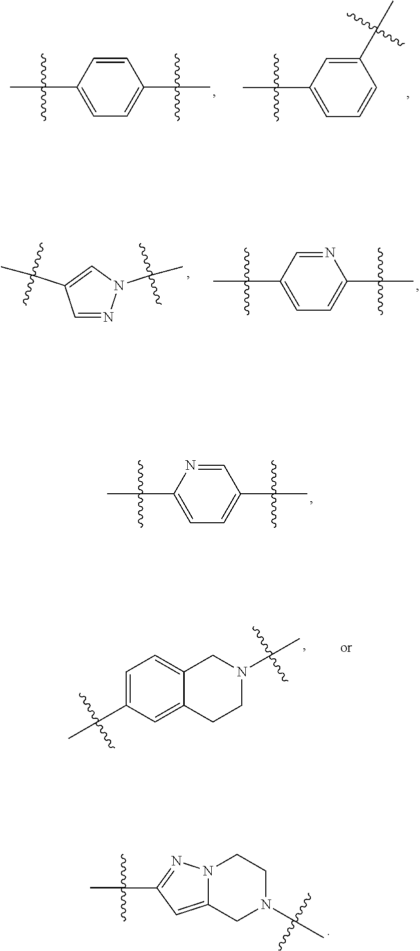

In some embodiments, X⁵ is —N(R)—.

In some embodiments, X⁵ is —C(O)—N(R)—.

In some embodiments, X⁵ is a bond.

In some embodiments, L is

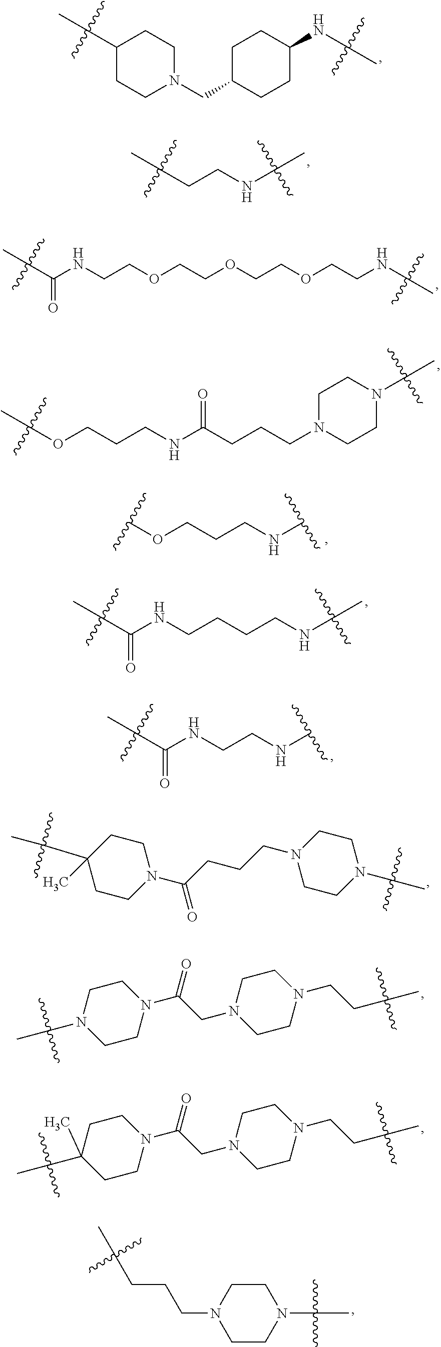

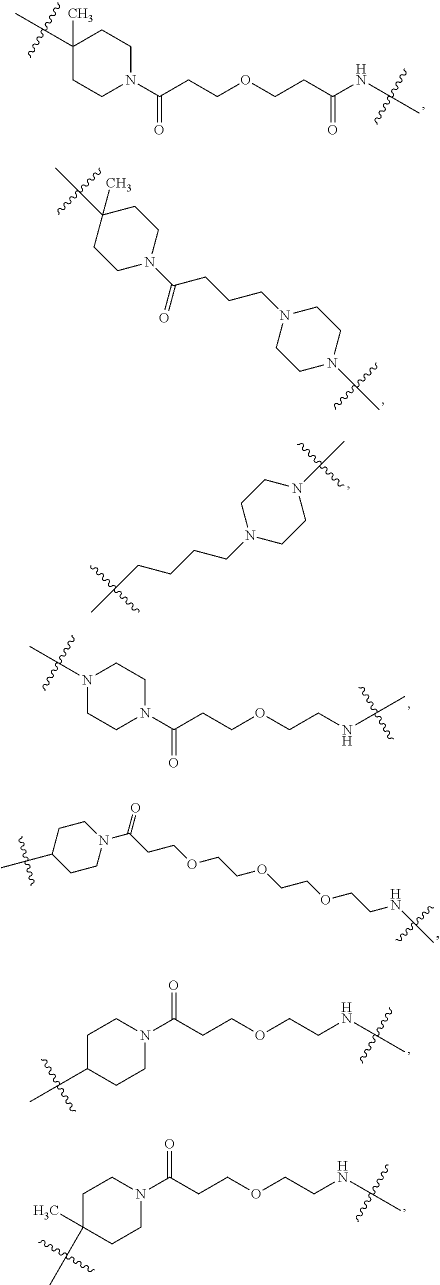

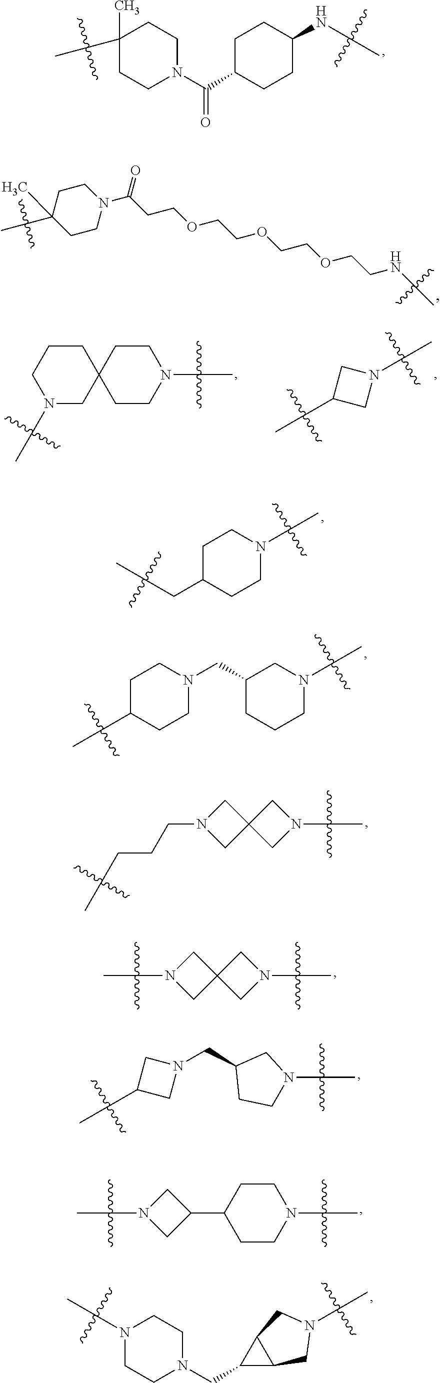

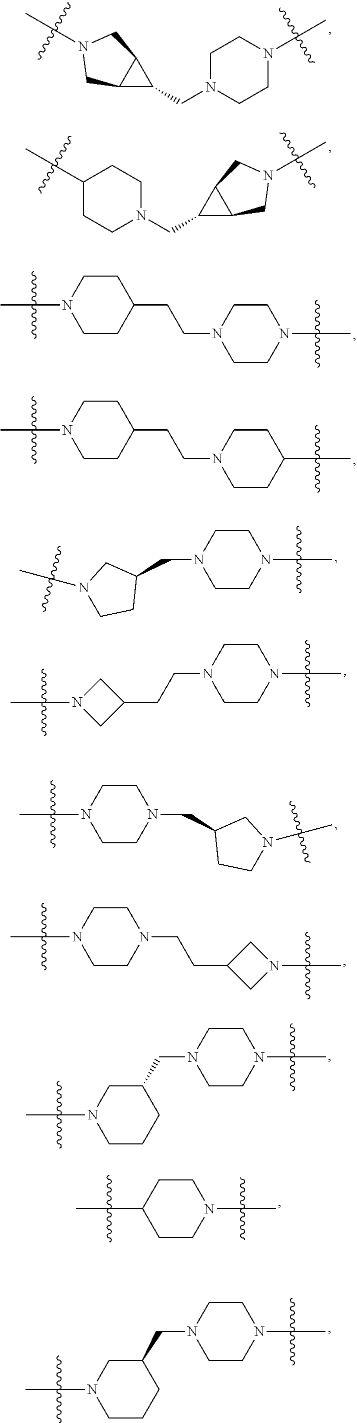

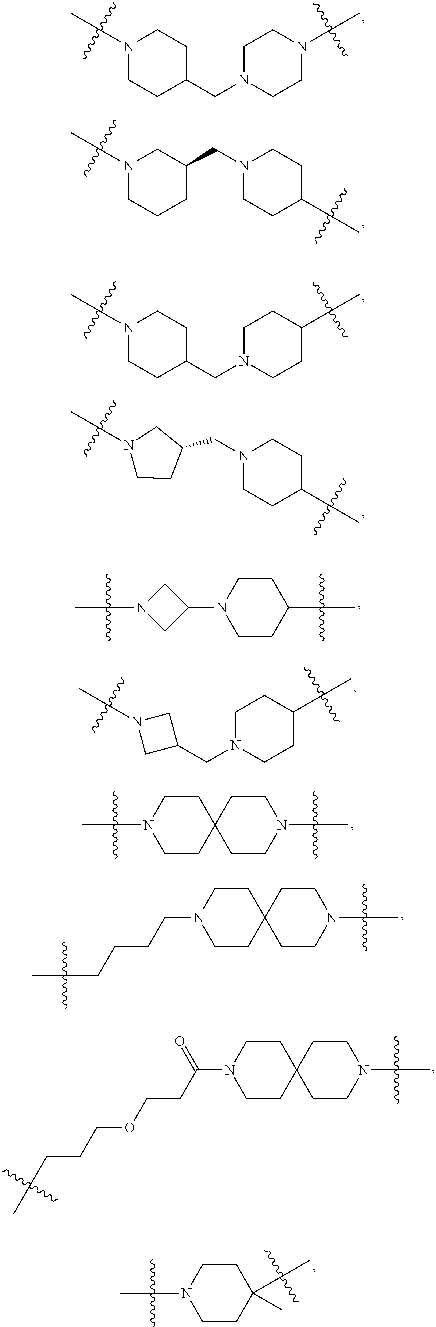

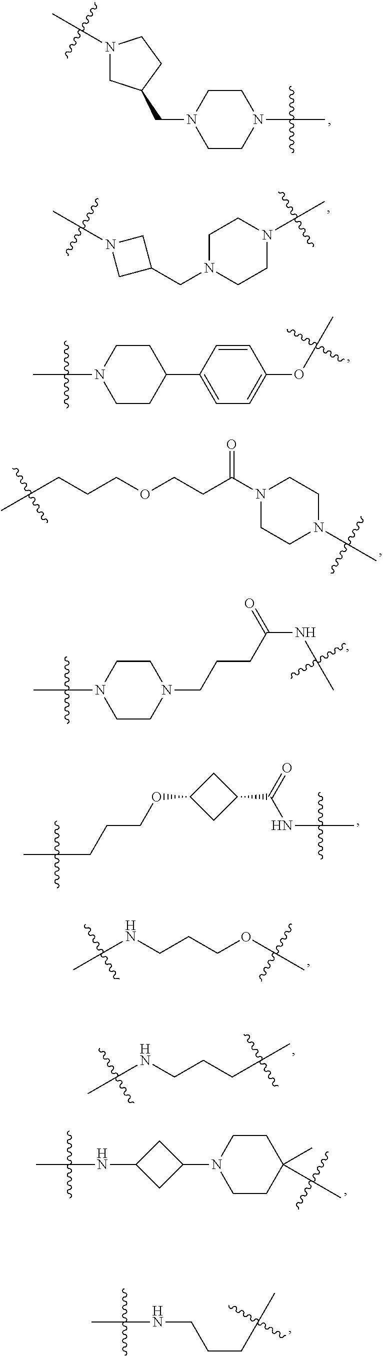

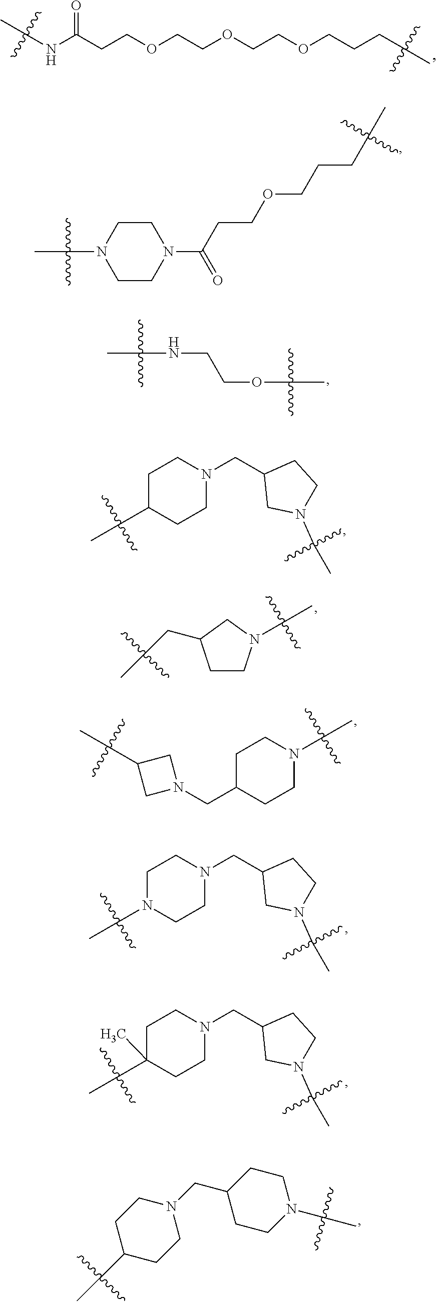

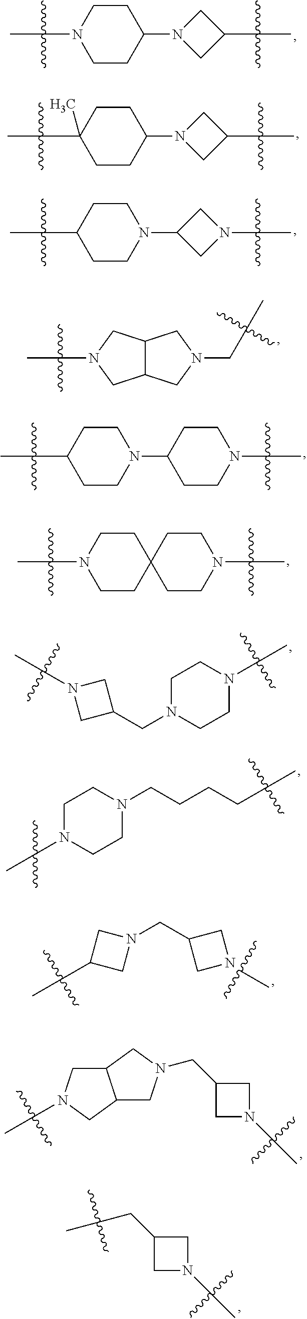

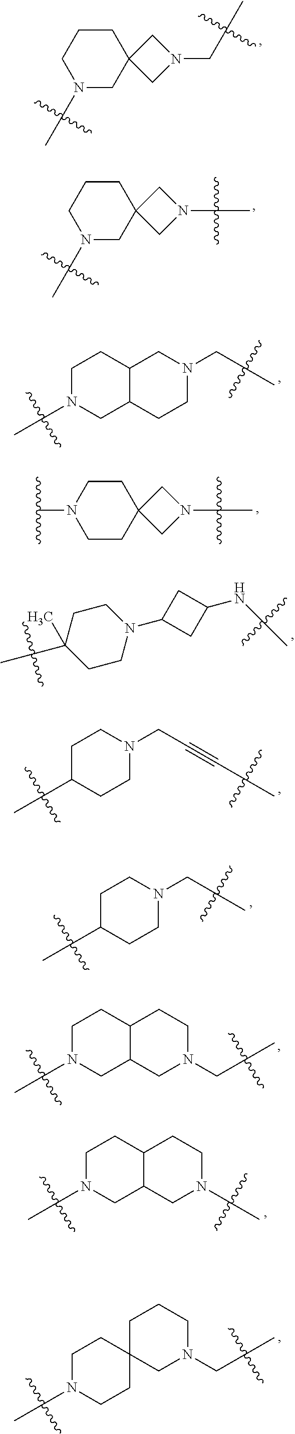

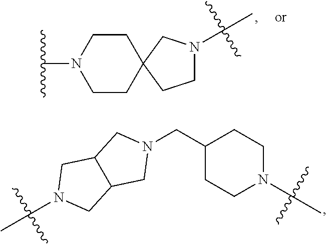

In some embodiments, Y is

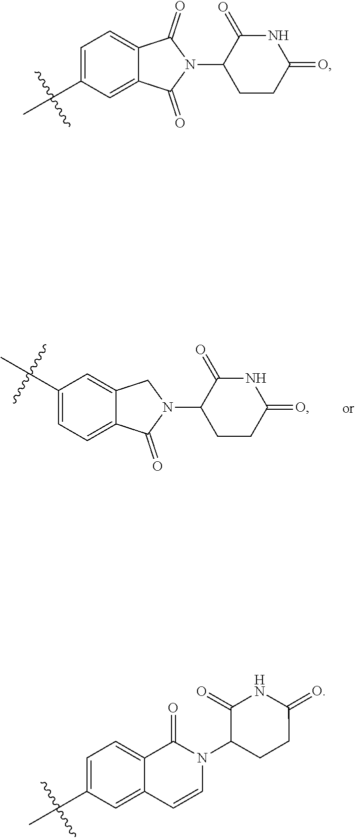 [SMILES: *c1ccc2c(c1)C(=O)N(C1CCC(=O)NC1=O)C2=O ; *c1ccc2c(c1)CN(C1CCC(=O)NC1=O)C2=O ; *c1ccc2c(=O)n(C3CCC(=O)NC3=O)ccc2c1]

The present invention also provides a compound of Formula (F)

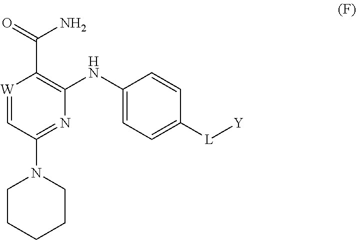

or a pharmaceutically acceptable salt thereof, wherein W is CH or N; L is —X¹—X²—X³—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having 1-3 heteroatoms independently selected from N, O, or S, or 4-6 membered monocyclic heterocycloalkyl having 1-2 heteroatoms independently selected from N, O, or S, wherein each of the monocyclic and bicyclic heterocycloalkyl of X¹ is optionally substituted with —CH₃; X² is a bond, —C₁₋₅ alkyl-, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having 1-2 heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having 1-2 heteroatoms independently selected from N, O, or S wherein the heterocycloalkyl is optionally substituted with —CH₃; each R is independently —H or —C₁₋₃ alkyl; each of m, n, and p is independently an integer from 1 to 3; and Y is

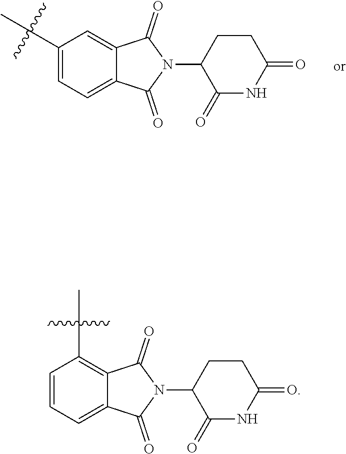 [SMILES: *c1ccc2c(c1)C(=O)N(C1CCC(=O)NC1=O)C2=O ; *c1cccc2c1C(=O)N(C1CCC(=O)NC1=O)C2=O]

In some embodiments, W is N.

In some embodiments, Y is

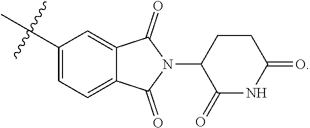 [SMILES: *c1ccc2c(c1)C(=O)N(C1CCC(=O)NC1=O)C2=O]

In some embodiments, X¹ is a 4-6 membered monocyclic heterocycloalkyl having 1-2 heteroatoms independently selected from N, O, or S, wherein each of the monocyclic heterocycloalkyl of X¹ is optionally substituted with —CH₃. For example, X¹ is

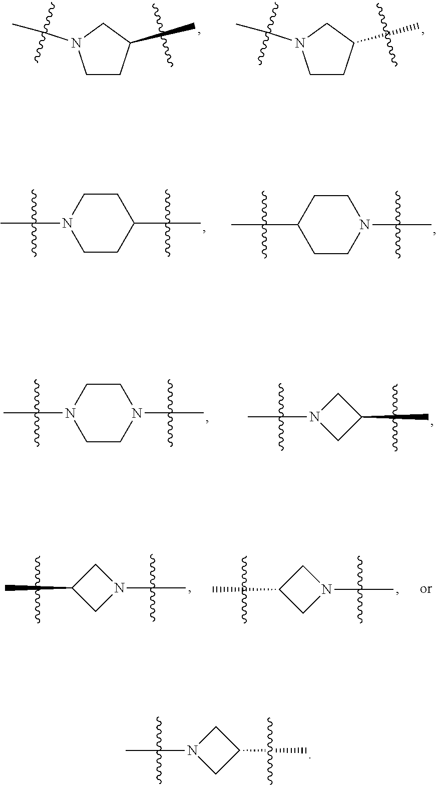

In some instances, X¹ is

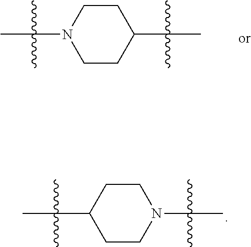 [SMILES: *C1CCN(*)CC1 ; *C1CCN(*)CC1]

In some embodiments, X² is a bond or —C₁₋₅ alkyl-.

In some embodiments, X³ is a 4-6 membered monocyclic heterocycloalkyl having 1-2 heteroatoms independently selected from N, O, or S. For example, X³ is

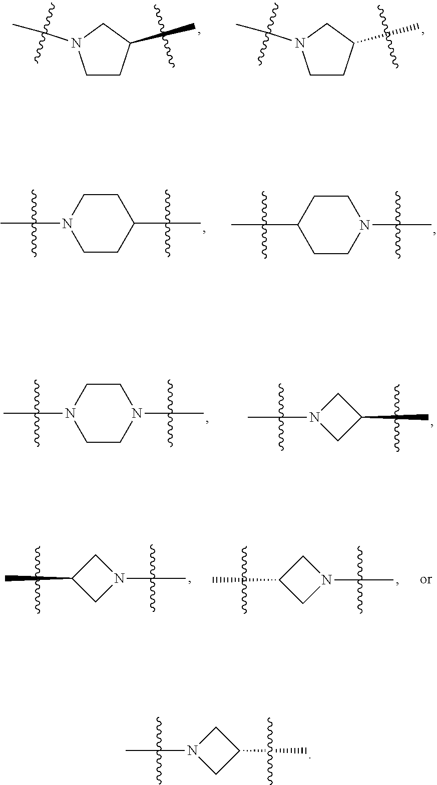

In some instances X³ is

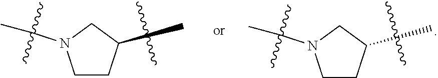

In some embodiments, L is

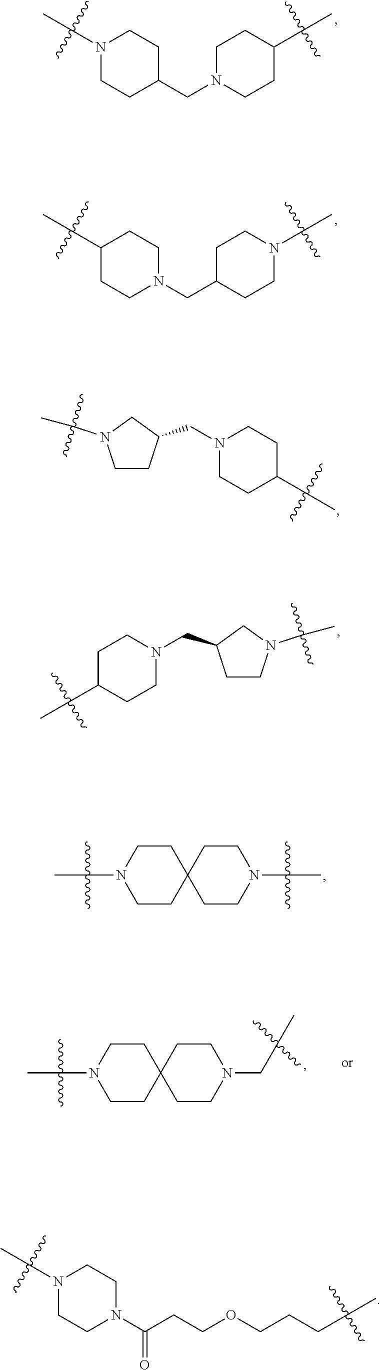

In some embodiments, L is

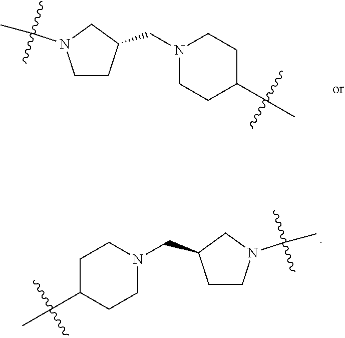 [SMILES: *C1CCN(C[C@@H]2CCN(*)C2)CC1 ; *C1CCN(C[C@@H]2CCN(*)C2)CC1]

In some embodiments, W is N and L is

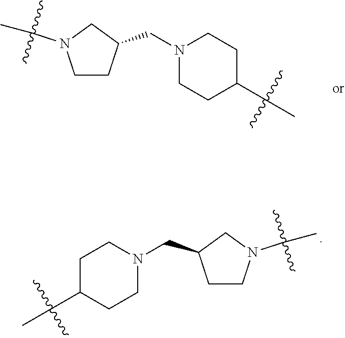 [SMILES: *C1CCN(C[C@@H]2CCN(*)C2)CC1 ; *C1CCN(C[C@@H]2CCN(*)C2)CC1]

The present invention also provides a compound of Formula (G)

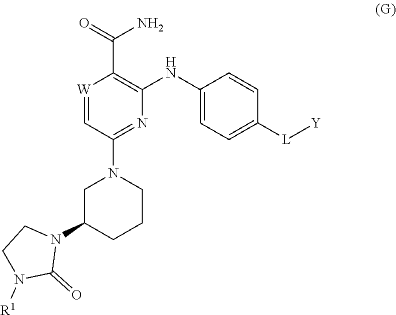

or a pharmaceutically acceptable salt thereof, wherein R¹, L, and Y are as defined for compounds of Formula (A).

In some embodiments, R¹ is methyl.

In some embodiments, Y is

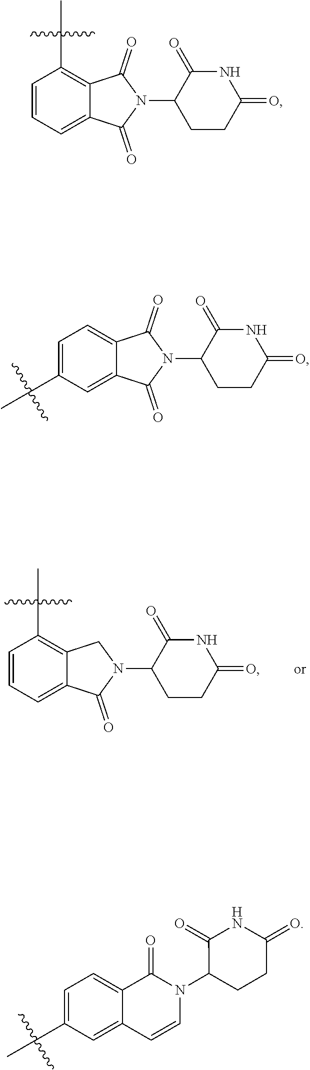 [SMILES: *c1cccc2c1C(=O)N(C1CCC(=O)NC1=O)C2=O ; *c1ccc2c(c1)C(=O)N(C1CCC(=O)NC1=O)C2=O ; *c1cccc2c1CN(C1CCC(=O)NC1=O)C2=O ; *c1ccc2c(=O)n(C3CCC(=O)NC3=O)ccc2c1]

In some embodiments, W is N.

The present invention also provides a compound of Formula (H)

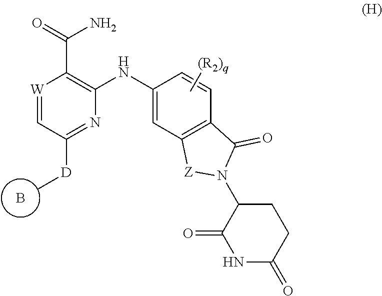

or a pharmaceutically acceptable salt thereof, wherein ring B, R², Z, W, D, and q are as defined in the compound of Formula (A).

In some embodiments, q is 0.

The present invention also provides a compound of Formula (J)

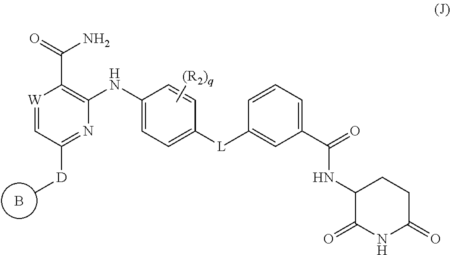

or a pharmaceutically acceptable salt thereof, wherein ring B, D, W, R², q, and L are as defined in the compound of Formula (A).

The present invention also provides a compound of Formula (K)

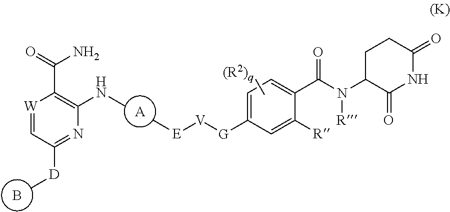

or a pharmaceutically acceptable salt thereof, wherein ring A is

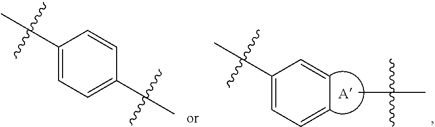

wherein ring A is optionally and independently substituted with up to 3 substituents selected from halo, —CN, -carboxyl, —NH₂, and optionally substituted —C₁₋₆ alkyl (e.g., optionally substituted —C₁₋₃ alkyl); V is a bond or —CH₂—; and E and G are each independently a 5-6 membered heterocycloalkyl, wherein each heterocycloalkyl contains at least one nitrogen atom. Ring B, W, R², q, R″, R′″, and ring A′ are as defined in the compound of Formula (A). In some embodiments, ring A′ together with the phenyl ring to which it is fused form a 9-10 membered bicyclic aryl or a 9-10 membered bicyclic heteroaryl wherein the bicyclic heteroaryl has 1-3 heteroatoms independently selected from N, O, or S.

In some embodiments, D is a bond and W is a nitrogen atom.

The present invention also provides a compound of Formula (M)

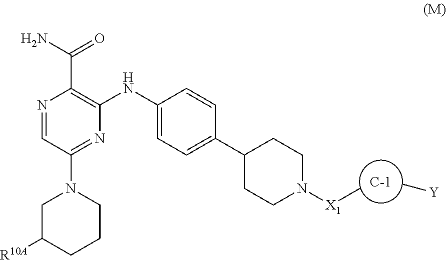

or a pharmaceutically acceptable salt thereof, wherein R^{10A} is —H,

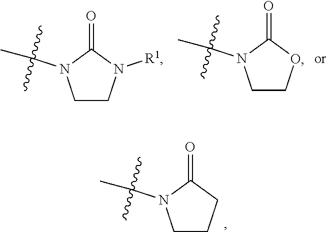

wherein R¹ is C₁₋₄ alkyl; X¹ is —C₁₋₅ alkyl-; ring C-1 is a 5-6 membered heterocycloalkyl having 1 nitrogen atom; and Y is

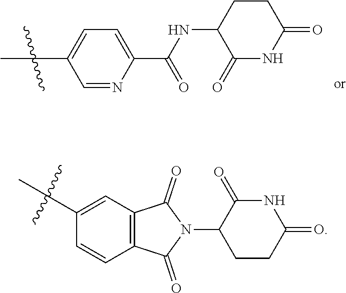 [SMILES: *c1ccc(C(=O)NC2CCC(=O)NC2=O)nc1 ; *c1ccc2c(c1)C(=O)N(C1CCC(=O)NC1=O)C2=O]

In some embodiments, R^{10A} is —H or

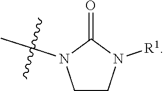

In some embodiments, R^{10A} is

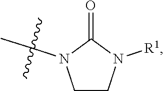

and R¹ is methyl, ethyl, propyl, iso-propyl, butyl, sec-butyl, or iso-butyl. For example, R¹ is methyl.

In some embodiments, X¹ is methylene, ethylene, or propylene. For instance, X¹ is methylene.

In some embodiments, ring C-1 is

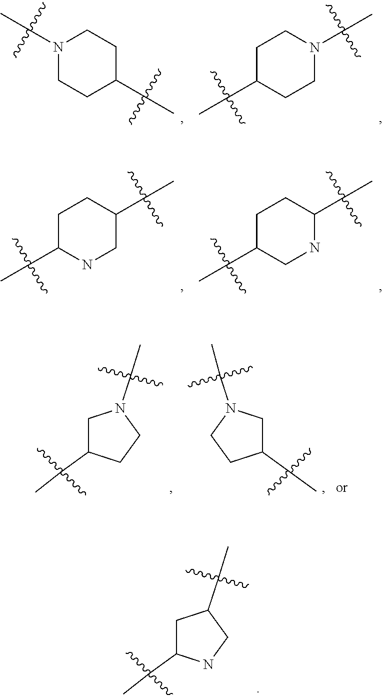

For instance, ring C-1 is

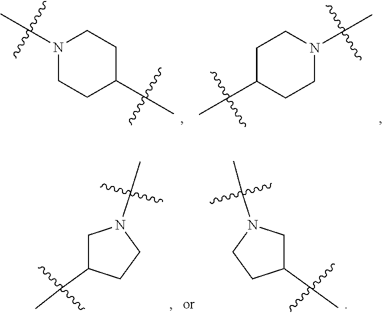 [SMILES: *C1CCN(*)CC1 ; *C1CCN(*)CC1 ; *C1CCN(*)C1 ; *C1CCN(*)C1]

The present invention provides a compound of Formula (X)

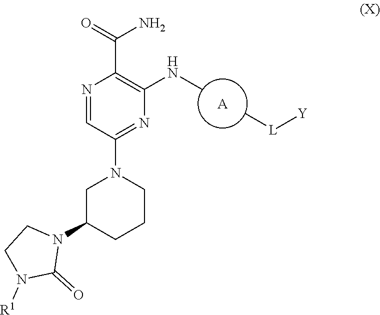

or a pharmaceutically acceptable salt thereof, wherein R¹ is C₁₋₃ alkyl; ring A is phenyl, 5-6 membered partially or fully unsaturated monocyclic heterocycle, 9-10 membered bicyclic aryl, or 9-10 membered bicyclic heteroaryl, wherein the heterocycle and the bicyclic heteroaryl of ring A each independently have 1-3 heteroatoms independently selected from N, O, or S; L is —X¹—X²—X³—X⁴—X⁵—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having 1-3 heteroatoms independently selected from N, O, or S wherein the bicyclic heterocycloalkyl of X¹ is optionally substituted with —CH₃, or 4-6 membered monocyclic heterocycloalkyl having 1-2 heteroatoms independently selected from N, O, or S wherein the monocyclic heterocycloalkyl of X¹ is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered monocyclic cycloalkyl, or 4-6 membered monocyclic heterocycloalkyl having 1-2 heteroatoms independently selected from N, O, or S; X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having 1-2 heteroatoms independently selected from N, O, or S wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated carbocycle having 0-3 heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from 1 to 3; Y is

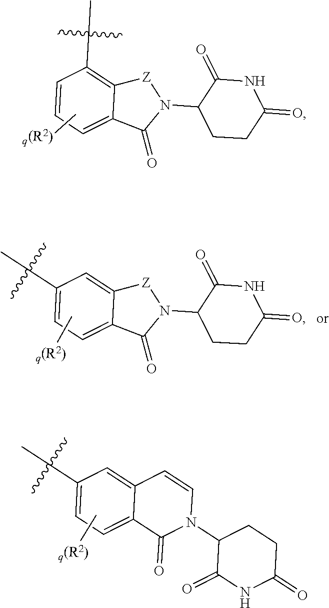

wherein; each R² is independently halo or C₁₋₄ alkyl; each Z is —C(R^{A})₂— or —C(O)—; each R^{A} is independently —H or C₁₋₄ alkyl; and q is 0, 1, or 2.

In some instances, the compound of Formula (X) is a compound of Formula (I)

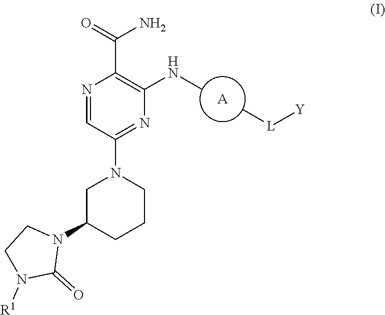

or a pharmaceutically acceptable salt thereof, wherein R¹ is C₁₋₃ alkyl; ring A is phenyl, 9-10 membered bicyclic aryl, or 9-10 membered bicyclic heteroaryl having 1-3 heteroatoms independently selected from N, O, or S; L is —X¹—X²—X³—X⁴—X⁵—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having 1-3 heteroatoms independently selected from N, O, or S wherein the heterocycloalkyl is optionally substituted with —CH₃, or 4-6 membered heterocycloalkyl having 1-2 heteroatoms independently selected from N, O, or S wherein the heterocycloalkyl is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having 1-2 heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, 4-6 membered heterocycloalkyl having 1-2 heteroatoms independently selected from N, O, or S wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having 1-3 heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from 1 to 3 (e.g., 1, 2, or 3); Y is

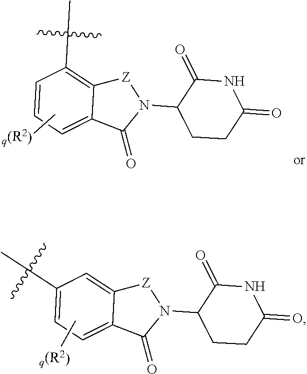

wherein each R² is independently halo or —C₁₋₄ alkyl; each Z is —C(R^{A})₂— or —C(O)—; each R^{A} is independently —H or —C₁₋₄ alkyl; and q is 0, 1, or 2.

In some embodiments, q is 0. In other embodiments, q is 1 and R² is —F.

In some embodiments, Z is —CH₂— or —C(O)—.

In some embodiments, Y is

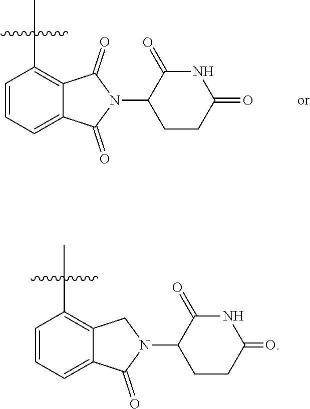 [SMILES: *c1cccc2c1C(=O)N(C1CCC(=O)NC1=O)C2=O ; *c1cccc2c1CN(C1CCC(=O)NC1=O)C2=O]

In other embodiments, Y is

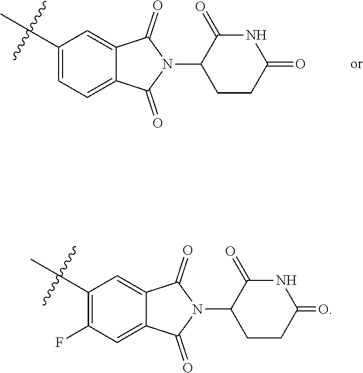 [SMILES: *c1ccc2c(c1)C(=O)N(C1CCC(=O)NC1=O)C2=O ; *c1cc2c(cc1F)C(=O)N(C1CCC(=O)NC1=O)C2=O]

In some embodiments, R¹ is —C₁₋₃ alkyl. For example, R¹ is methyl, ethyl, propyl, or iso-propyl. In other examples, R¹ is methyl.

In some embodiments, each R is independently —H or —CH₃. For instance, each R is —H.

In some embodiments, X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having 1-3 heteroatoms independently selected from N, O, or S, or 4-6 membered heterocycloalkyl having 1-2 heteroatoms independently selected from N, O, or S wherein the heterocycloalkyl is optionally substituted with —CH₃. In some embodiments, X¹ is —C(O)—N(R)—. For example, X¹ is —C(O)—N(H)—, —C(O)—N(CH₃)—, or —C(O)—N(CH₂CH₃)—. In other embodiments, X¹ is a 5-6 membered heterocycloalkyl having 1-2 heteroatoms independently selected from N, O, or S wherein the heterocycloalkyl is optionally substituted with —CH₃. For example, X¹ is,

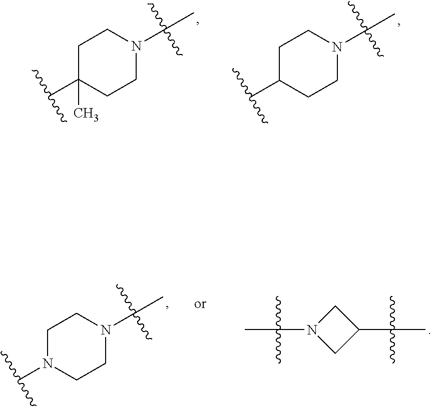

In other examples, X¹ is a 7-10 membered spiro bicyclic heterocycloalkyl ring having 1-3 heteroatoms independently selected from N, O, or S (e.g., N). For example, X¹ is

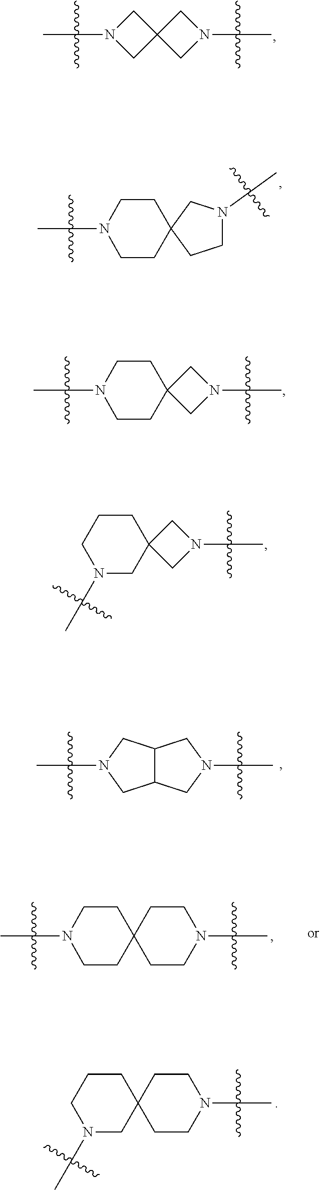

In other embodiments, X¹ is —(O—CH₂—CH₂)ₘ— or —(O—CH₂—CH₂—CH₂)ₘ—, wherein m is 1, 2, or 3. For example, X¹ is —(O—CH₂—CH₂)ₘ— or —(O—CH₂—CH₂—CH₂)ₘ—, and m is 1. In another example, X¹ is —(O—CH₂—CH₂)ₘ— or —(O—CH₂—CH₂—CH₂)ₘ—, and m is 2. In some embodiments, X¹ is —C₁₋₅ alkyl-. For example, X¹ is methylene, ethylene, propylene, butylene, or the like. In some embodiments, X¹ is —CH₂—, —C(O)—,

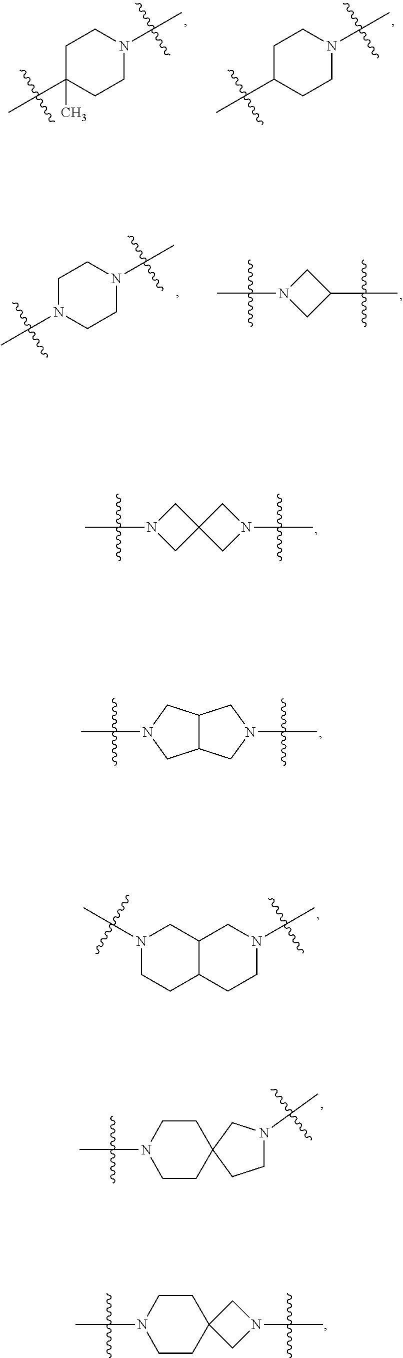

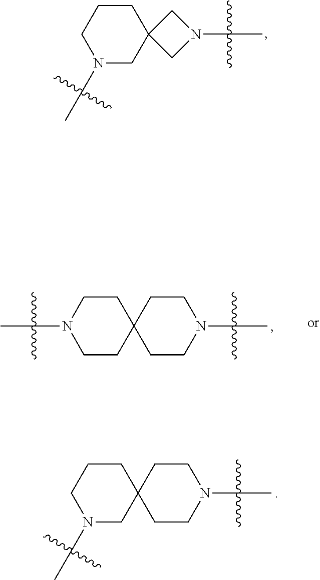 [SMILES: *N1CCCC2(C1)CN(*)C2 ; *N1CCC2(CC1)CCN(*)CC2 ; *N1CCC2(CCCN(*)C2)CC1]

In some embodiments, X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having 1-2 heteroatoms independently selected from N, O, or S. In some embodiments, X² is a bond. In some embodiments, X² is —(O—CH₂—CH₂)ₙ—, —(CH₂—CH₂—O)—, or —C₁₋₅ alkyl-, wherein n is 1, 2, or 3. For example, X¹ is —C(O)—N(R)—, and X² is —(O—CH₂—CH₂)ₙ—, —(CH₂—CH₂—O)ₙ—, or —C₁₋₅ alkyl-. In some examples, X² is —(O—CH₂—CH₂)ₙ— or —(CH₂—CH₂—O)ₙ—, where n is 1 or 2. In other examples, X² is —C₁₋₅ alkyl-. For instance, X² is methylene, ethylene, propylene, butylene, or the like. In other examples, X² is a bond, —CH₂—, —CH₂—CH₂—, or —CH₂—CH₂—CH₂—. In some examples, X² is 4-6 membered cycloalkyl. For instance, X² is

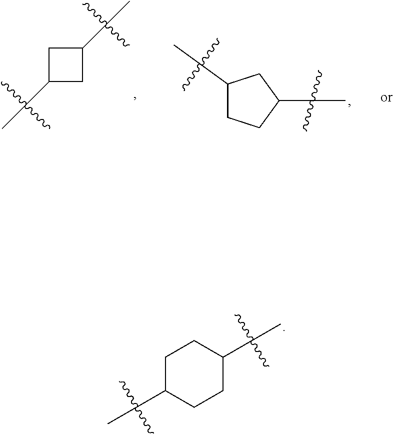 [SMILES: *C1CC(*)C1 ; *C1CCC(*)C1 ; *C1CCC(*)CC1]

In other examples X² is 4-6 membered heterocycloalkyl having 1-2 heteroatoms independently selected from N, O, or S. For instance, X² is

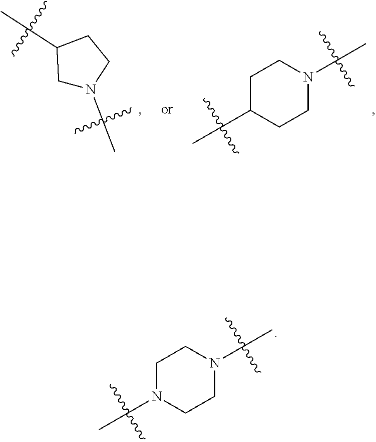 [SMILES: *C1CCN(*)C1 ; *C1CCN(*)CC1 ; *N1CCN(*)CC1]

In some embodiments, X³ is a bond, —C₁₋₄ alkyl-, 4-6 membered cycloalkyl, —N(R)—, —(O—CH₂—CH₂)ₚ—, —(CH₂—CH₂—O)ₚ—, 4-6 membered heterocycloalkyl having 1-2 heteroatoms independently selected from N, O, or S wherein the heterocycloalkyl is optionally substituted with —CH₃. In some embodiments, X³ is a bond. In some embodiment, X³ is methyl, ethyl, propyl, iso-propyl, butyl, or the like. In some embodiments, X³ is cyclopently or cyclohexyl. In some embodiments, X³—N(H)—. And, in other embodiments, X³ is —(O—CH₂—CH₂)ₚ— or —(CH₂—CH₂—O)ₚ—, wherein p is 1 or 2.

In some embodiments, X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having 1-3 heteroatoms independently selected from N, O, or S. In some embodiments, X⁴ is a bond,

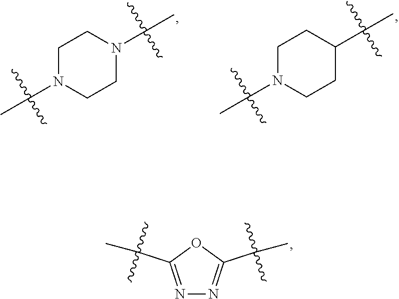 [SMILES: *N1CCN(*)CC1 ; *C1CCN(*)CC1 ; *c1nnc(*)o1]

—C₁₋₄ alkyl-, —CH₂—CH₂—N(R)—, or —N(R)—. For example, X⁴ is —CH₂—CH₂—N(H)—, or —N(H)—. In other examples, X⁴ is methyl, ethyl, propyl, iso-propyl, butyl, sec-butyl, or the like.

In some embodiments, X⁵ is a bond, —C₁₋₄ alkyl-, —N(R)—, or —C(O)—N(R)—. In some embodiments, X⁵ is a bond. In some embodiments, X⁵ is methyl, ethyl, propyl, iso-propyl, butyl, or the like. In some embodiments, X⁵ is —N(H)— or —C(O)—N(H)—.

In some embodiments, L is selected from

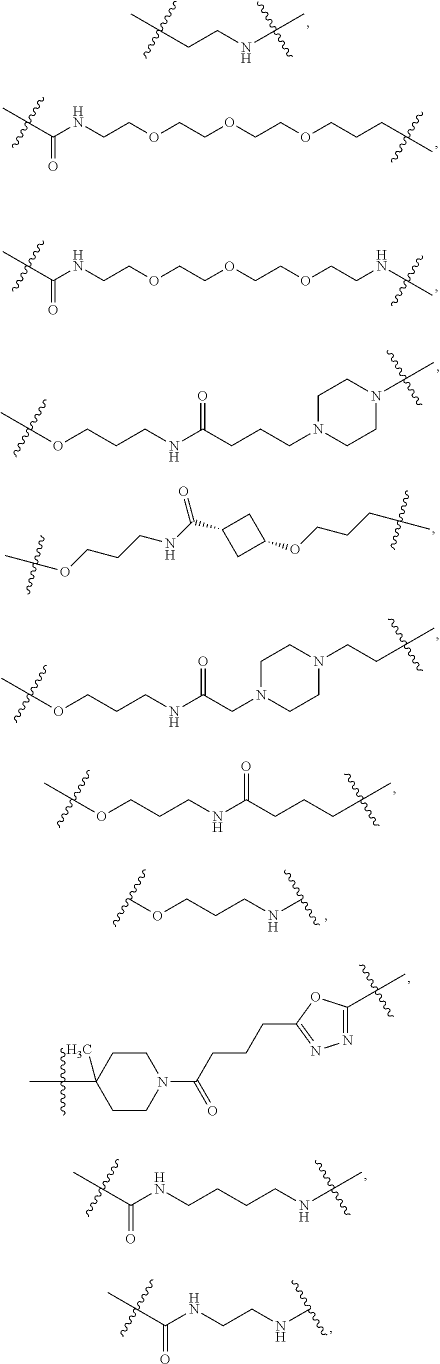

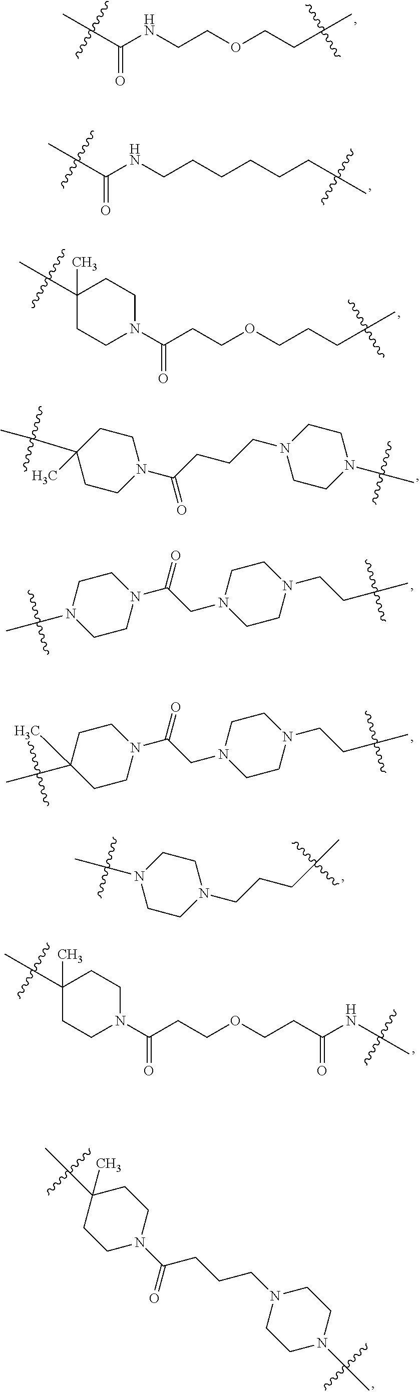

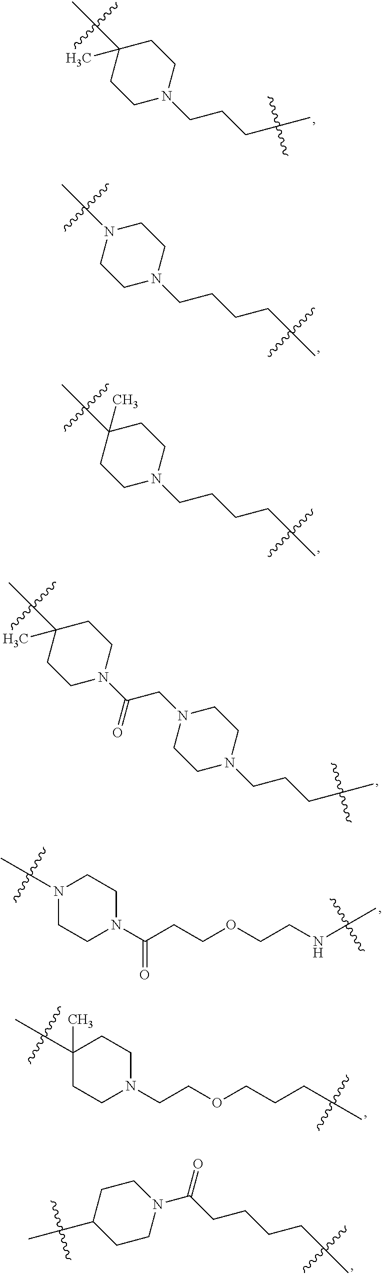

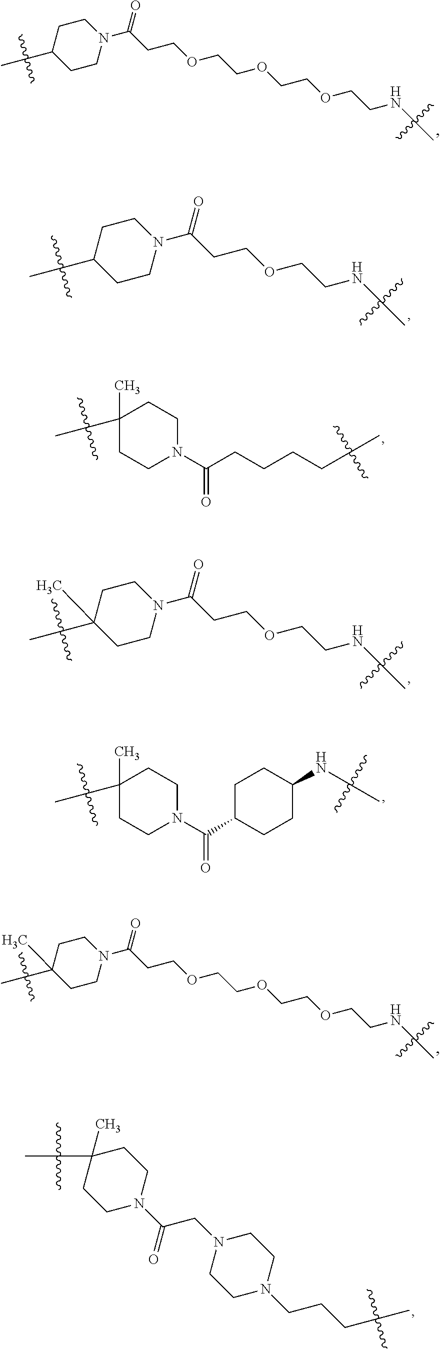

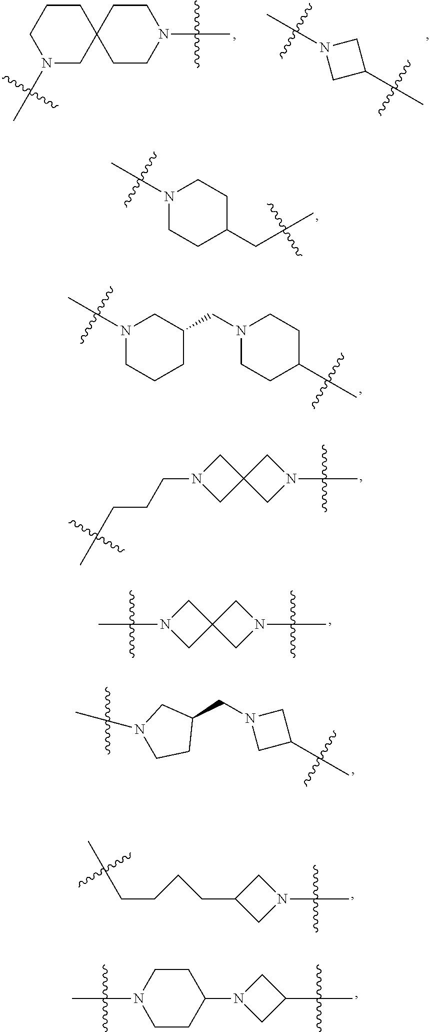

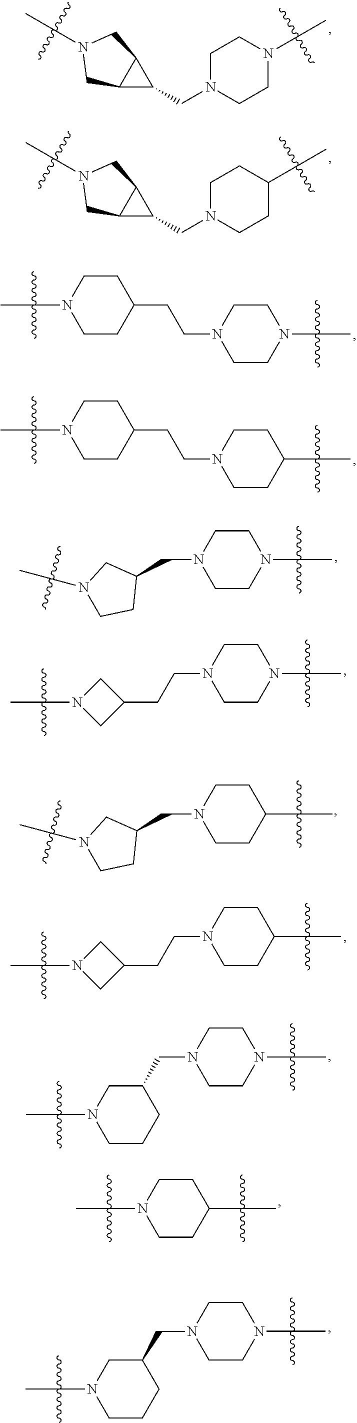

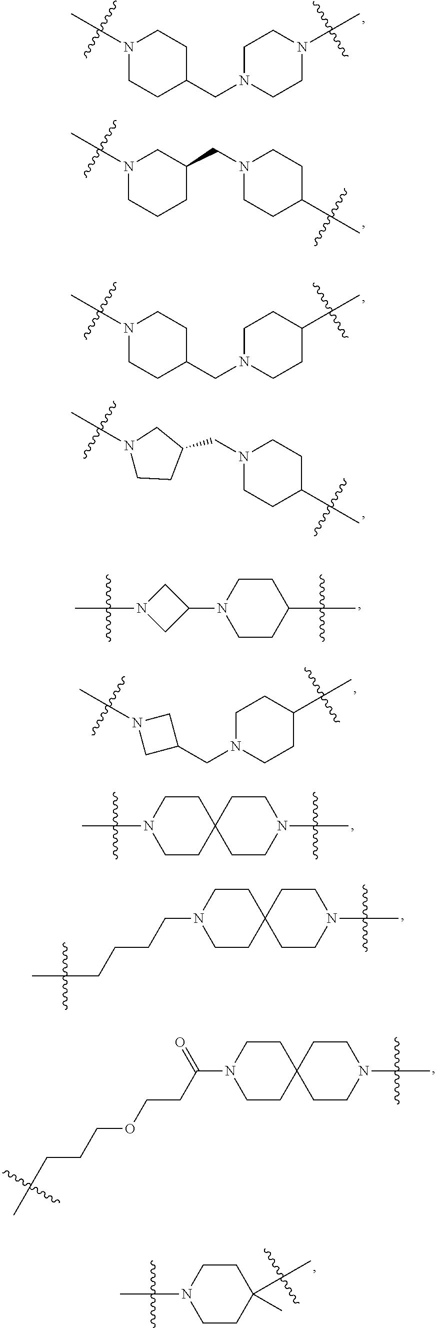

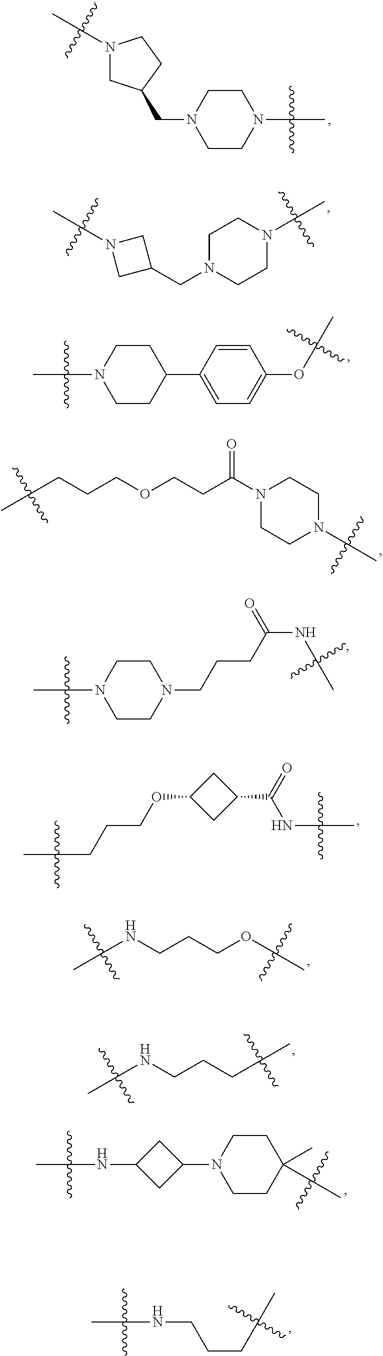

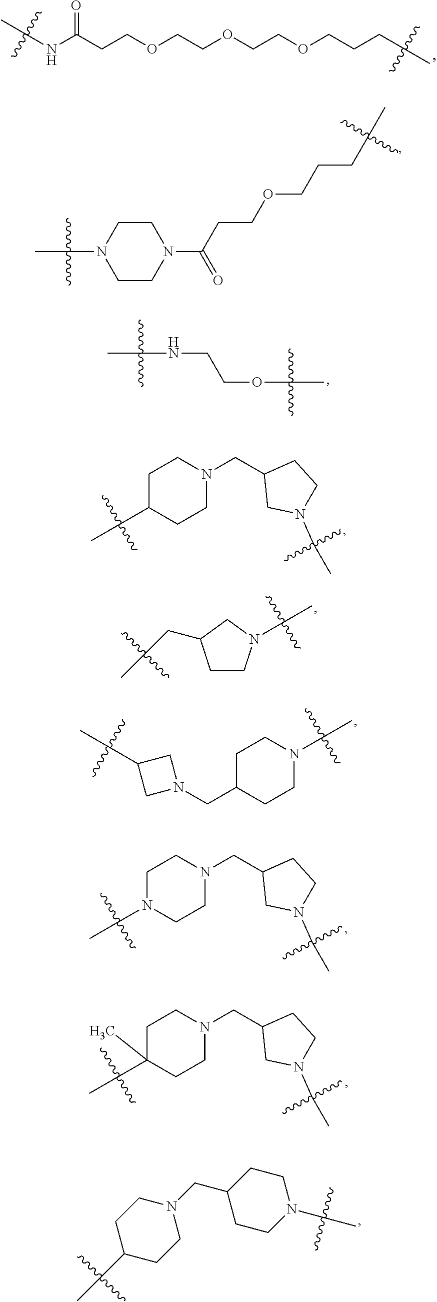

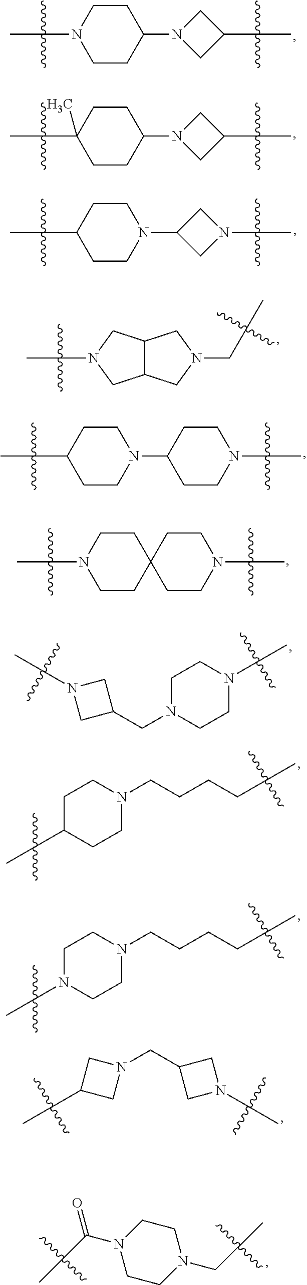

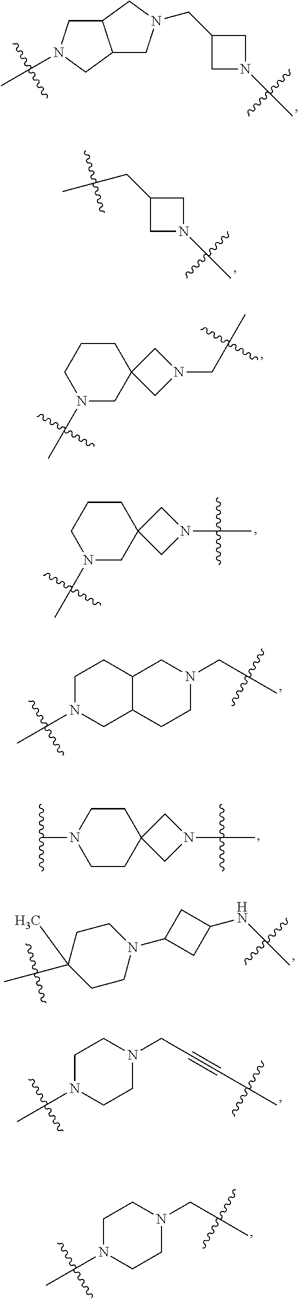

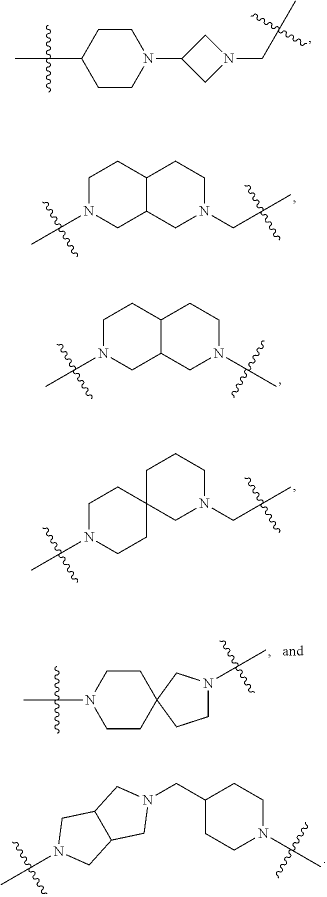 [SMILES: *CN1CC(N2CCC(*)CC2)C1 ; *CN1CCC2CCN(*)CC2C1 ; *N1CCC2CCN(*)CC2C1 ; *CN1CCCC2(CCN(*)CC2)C1 ; *N1CCC2(CC1)CCN(*)C2 ; *N1CCC(CN2CC3CN(*)CC3C2)CC1]

The present invention also provides a compound of Formula (I-A):

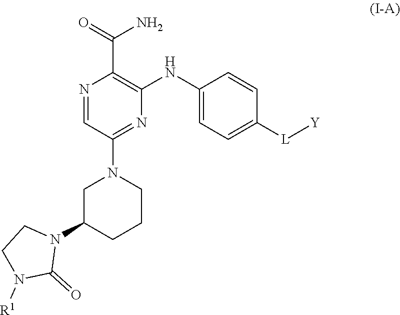

or a pharmaceutically acceptable salt thereof, wherein R¹ is C₁₋₃ alkyl; L is —X¹—X²—X³—X⁴—X⁵—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having 1-3 heteroatoms independently selected from N, O, or S wherein the heterocycloalkyl is optionally substituted with —CH₃, or 4-6 membered heterocycloalkyl having 1-2 heteroatoms independently selected from N, O, or S wherein the heterocycloalkyl is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having 1-2 heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, or 4-6 membered heterocycloalkyl having 1-2 heteroatoms independently selected from N, O, or S wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having 1-3 heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from 1 to 3; Y is

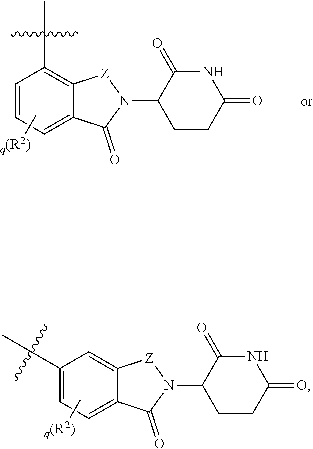

wherein each R² is independently halo or —C₁₋₄ alkyl; each Z is —C(R^{A})₂— or —C(O)—; each R^{A} is independently —H or —C₁₋₄ alkyl; and q is 0, 1, or 2.

In other embodiments, each of the variables in Formula (I-A) is as defined herein for the compound of Formula (X) or (I).

The present invention also provides a compound of Formula (I-B)

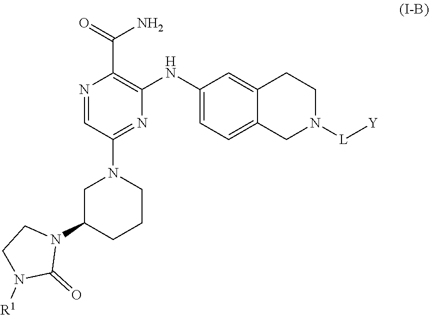

or a pharmaceutically acceptable salt thereof, wherein R¹ is C₁₋₃ alkyl; L is —X¹—X²—X³—X⁴—X⁵—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl ring having 1-3 heteroatoms independently selected from N, O, or S wherein the heterocycloalkyl is optionally substituted with —CH₃, or 4-6 membered heterocycloalkyl having 1-2 heteroatoms independently selected from N, O, or S wherein the heterocycloalkyl is optionally substituted with —CH₃; X² is a bond, —(O—CH₂—CH₂)ₙ—, —(CH₂—CH₂—O)—, —N(R)—C(O)—, —N(R)—, —C(O)—, —C₁₋₅ alkyl-, 4-6 membered cycloalkyl, or 4-6 membered heterocycloalkyl having 1-2 heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, or 4-6 membered heterocycloalkyl having 1-2 heteroatoms independently selected from N, O, or S wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having 1-3 heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from 1 to 3; Y is

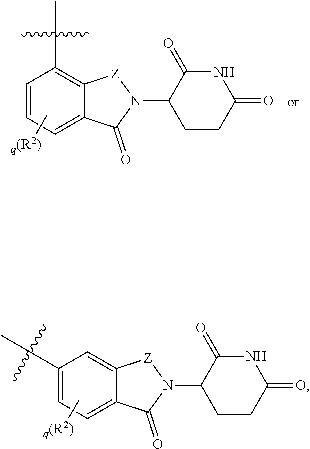

wherein each R² is independently halo or C₁₋₄ alkyl; each Z is —C(R^{A})₂— or —C(O)—; each R^{A} is independently —H or C₁₋₄ alkyl; and q is 0, 1, or 2.

In other embodiments, each of the variables in Formula (I-B) is as defined herein for the compound of Formula (X) or (I).

The present invention also provides a compound of Formula (II):

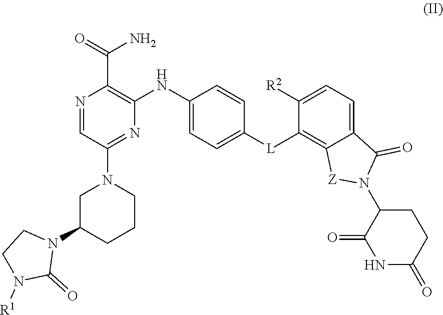

or a pharmaceutically acceptable salt thereof, wherein each of R¹, R², L, and Z are as defined herein for the compound of Formula (X), (I), (I-A), or (I-B).

In some embodiments, the compound of Formula (II) is a compound of Formulae (II-A) or (II-B)

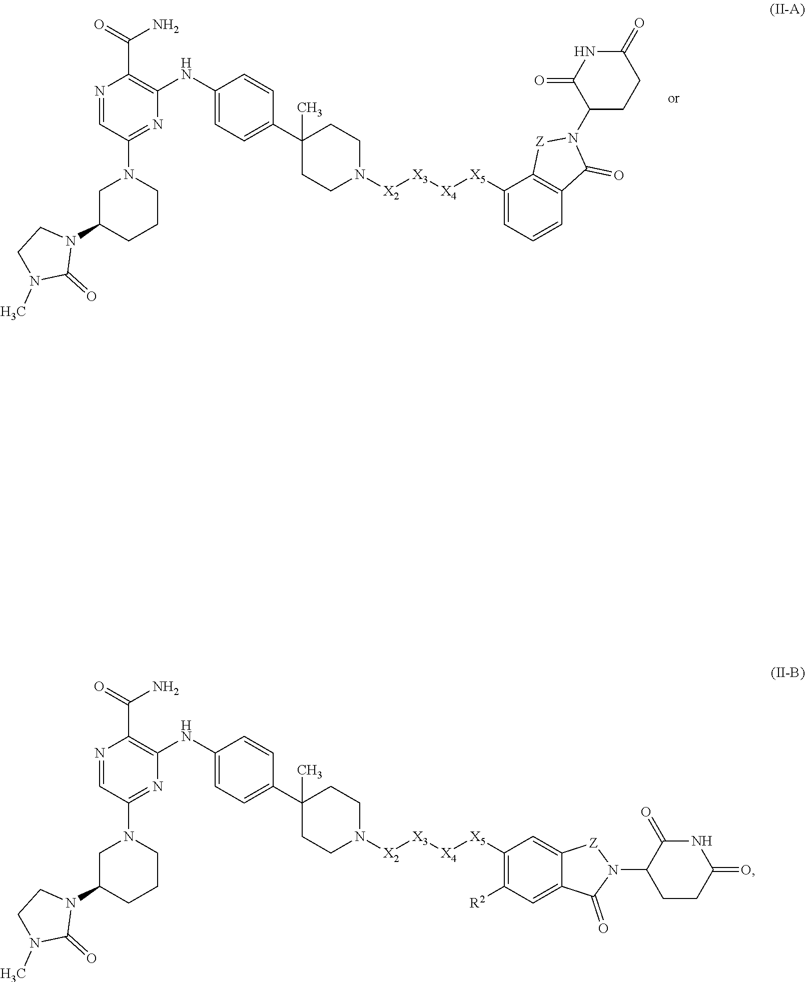

or a pharmaceutically acceptable salt thereof, wherein each of X², X³, X⁴, and X⁵ are as defined herein for the compound of Formula (X), (I), (I-A), (I-B), or (II).

The present invention also provides a compound of Formula (III)

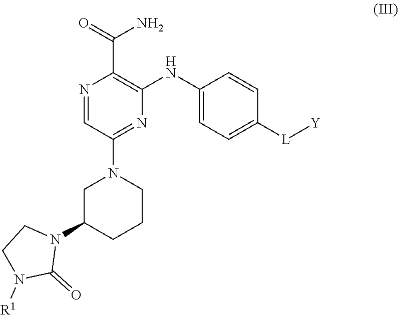

or a pharmaceutically acceptable salt thereof, wherein R¹ is C₁₋₃ alkyl; L is —X¹—X²—X³—; X¹ is 7-12 membered spiro bicyclic heterocycloalkyl having 1-3 heteroatoms independently selected from N, O, or S wherein the heterocycloalkyl is optionally substituted with —CH₃, or 4-6 membered heterocycloalkyl having 1-2 heteroatoms independently selected from N, O, or S wherein the heterocycloalkyl is optionally substituted with —CH₃; X² is a bond or —C₁₋₅ alkyl-; X³ is a bond, —C₁₋₄ alkyl-,4-6 membered heterocycloalkyl having 1-2 heteroatoms independently selected from N, O, or S wherein the heterocycloalkyl is optionally substituted with —CH₃; Y is

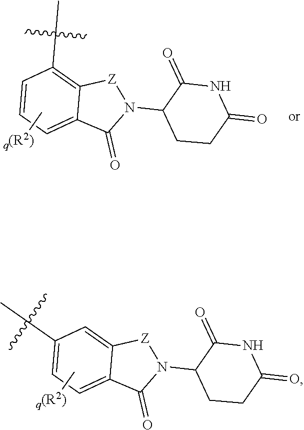

wherein each R² is independently halo or —C₁₋₄ alkyl; each Z is —C(R^{A})₂— or —C(O)—; each R^{A} is independently —H; and q is 0, 1, or 2.

The present invention also provides a compound of Formula (IV)

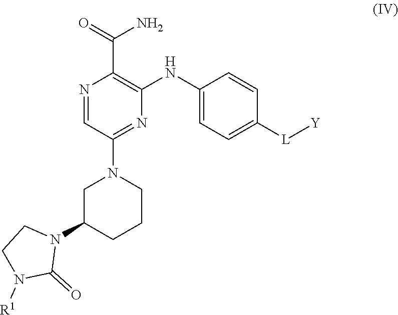

or a pharmaceutically acceptable salt thereof, wherein R¹ is C₁₋₃ alkyl; L is —X¹—X²—X³—X⁴—X⁵—; X¹ is —C(O)—N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having 1-3 heteroatoms independently selected from N, O, or S wherein the heterocycloalkyl is optionally substituted with —CH₃, or 4-6 membered heterocycloalkyl having 1-2 heteroatoms independently selected from N, O, or S wherein the heterocycloalkyl is optionally substituted with —CH₃; X² is a bond, —(O—CH₂—CH₂)ₙ—, —(CH₂—CH₂—O)ₙ—, —N(R)—C(O)—, —N(R)—, —C(O)—, —C₁₋₅ alkyl-, 4-6 membered cycloalkyl, or 4-6 membered heterocycloalkyl having 1-2 heteroatoms independently selected from N, O, or S; X³ is a bond, —C₁₋₄ alkyl-, 4-6 membered cycloalkyl, —N(R)—, —(O—CH₂—CH₂)ₚ—, —(CH₂—CH₂—O)ₚ—, or 4-6 membered heterocycloalkyl having 1-2 heteroatoms independently selected from N, O, or S wherein the heterocycloalkyl is optionally substituted with —CH₃; X⁴ is a bond, —CH₂—CH₂—N(R)—, —N(R)—, —C₁₋₄ alkyl-, —(O—CH₂—CH₂—CH₂)ₘ—, or 5-6 membered saturated, partially unsaturated, or fully unsaturated heterocycle having 1-3 heteroatoms independently selected from N, O, or S; X⁵ is a bond, —C₁₋₄ alkyl-, —N(R)—, or —C(O)—N(R)—; each R is independently —H or —C₁₋₃ alkyl; each of m, n, and p is independently an integer from 1 to 3; Y is

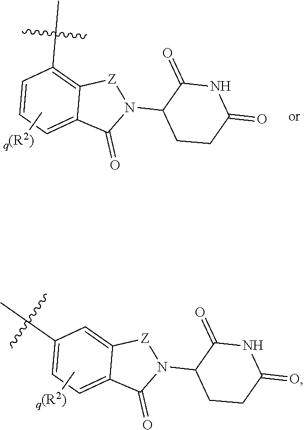

wherein each R² is independently halo or —C₁₋₄ alkyl; each Z is —C(R^{A})₂— or —C(O)—; each R^{A} is independently —H or —C₁₋₄ alkyl; and q is 0, 1, or 2.

B. General Synthetic Schemes

General Procedure 1: Amide Coupling.

A mixture of amine (0.03 mmol), acid (0.03 mmol), HATU (0.04 mmol), DIPEA (0.15 mmol) and DMF was allowed to stir at r.t. for 30 minutes. The mixture was purified by HPLC (H₂O/MeCN with 0.1% TFA) to afford the amide product. An exemplary amide coupling is provided in the Scheme 1 below where 3-(3-(2-(2,6-dioxopiperidin-3-yl)-1-oxoisoindolin-4-yl)propoxy)propanoic acid, and (R)-3-((4-(3,9-diazaspiro[5.5]undecan-3-yl)phenyl)amino)-5-(3-(3-methyl-2-oxoimidazolidin-1-yl)piperidin-1-yl)pyrazine-2-carboxamide were reacted as described above to provide 3-((4-(9-(3-(3-(2-(2,6-dioxopiperidin-3-yl)-1-oxoisoindolin-4-yl)propoxy)propanoyl)-3,9-diazaspiro[5.5]undecan-3-yl)phenyl)amino)-5-((R)-3-(3-methyl-2-oxoimidazolidin-1-yl)piperidin-1-yl)pyrazine-2-carboxamide (Compound 57).

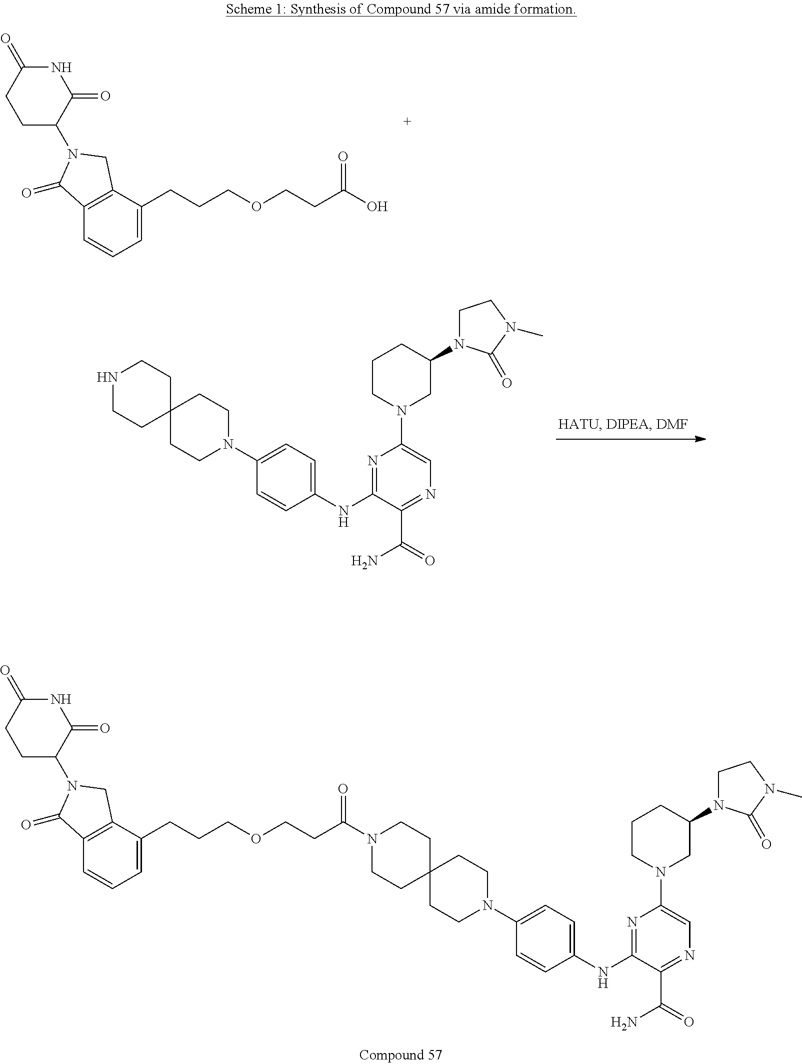 [SMILES: O=C(O)CCOCCCc1cccc2c1CN(C1CCC(=O)NC1=O)C2=O ; CN1CCN([C@@H]2CCCN(c3cnc(C(N)=O)c(Nc4ccc(N5CCC6(CCNCC6)CC5)cc4)n3)C2)C1=O ; CN1CCN([C@@H]2CCCN(c3cnc(C(N)=O)c(Nc4ccc(N5CCC6(CCN(C(=O)CCOCCCc7cccc8c7CN(C7CCC(=O)NC7=O)C8=O)CC6)CC5)cc4)n3)C2)C1=O]

Other amide containing compounds of the invention synthesized by using general procedure 1 are Compounds 2-9, 10-14, 19, 20, 22-28, 61, 62, 63, and 67.

General Procedure 2: Reductive Amination.

A mixture of amine TFA salt (0.07 mmol), aldehyde (0.1 mmol), triethylamine (0.28 mmol), and DCE were allowed to stir at r.t. for 10 minutes. NaBH(OAc)₃ (0.14 mmol) was added and the mixture was allowed to stir at r.t. for 2 h. The mixture was filtered through celite, washing with CH₂Cl₂, concentrated, and purified by HPLC (H₂O/MeCN with 0.1% TFA) to afford the amine product. An exemplary reductive amination is provided in Scheme 2 where (R)-5-(3-(3-methyl-2-oxoimidazolidin-1-yl)piperidin-1-yl)-3-((4-(piperidin-4-yl)phenyl)amino)pyrazine-2-carboxamide was reacted as described above with (3R)-1-(2-(2,6-dioxopiperidin-3-yl)-1,3-dioxoisoindolin-5-yl)piperidine-3-carbaldehyde to provide 3-((4-(1-(((3S)-1-(2-(2,6-dioxopiperidin-3-yl)-1,3-dioxoisoindolin-5-yl)piperidin-3-yl)methyl)piperidin-4-yl)phenyl)amino)-5-((R)-3-(3-methyl-2-oxoimidazolidin-1-yl)piperidin-1-yl)pyrazine-2-carboxamide (Compound 32).

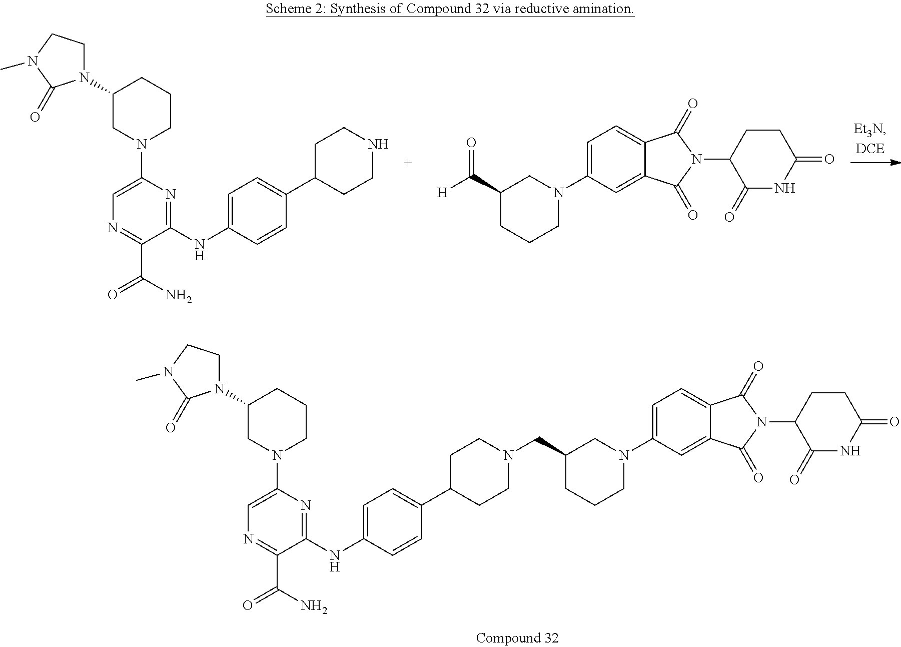 [SMILES: CN1CCN([C@@H]2CCCN(c3cnc(C(N)=O)c(Nc4ccc(C5CCNCC5)cc4)n3)C2)C1=O ; O=C[C@@H]1CCCN(c2ccc3c(c2)C(=O)N(C2CCC(=O)NC2=O)C3=O)C1 ; CN1CCN([C@@H]2CCCN(c3cnc(C(N)=O)c(Nc4ccc(C5CCN(C[C@@H]6CCCN(c7ccc8c(c7)C(=O)N(C7CCC(=O)NC7=O)C8=O)C6)CC5)cc4)n3)C2)C1=O]

Other amine containing compounds of the invention synthesized by using general procedure 2 are Compounds 33, 46, 56, 15-18, 21, 31, 48-52, 54, 59, 60, 35, 36, and 38-45.

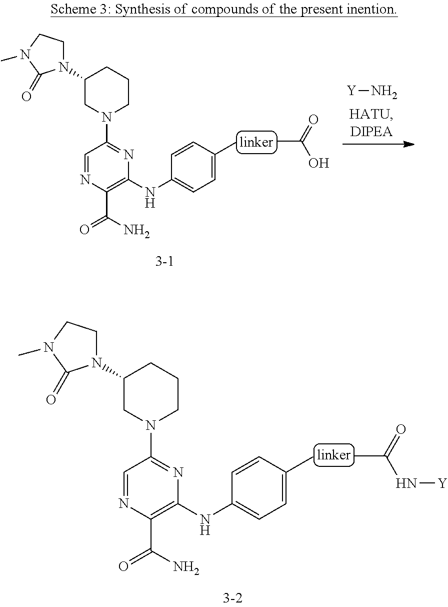

Intermediate (3-1), which can be generated by de-esterifying intermediate (1-6), is treated with amine, Y—NH₂, under coupling conditions to generate compounds of the present invention (3-2), wherein the terminal linking group of L is an amide.

General Procedure 3: Aryl Fluoride Displacement.

A mixture of amine (0.22 mmol), aryl fluoride (0.22 mmol), DIPEA (0.88 mmol) and DMF (1 mL) was allowed to stir at 90° C. for 16 h. The mixture was purified by HPLC (H₂O/MeCN with 0.1% TFA) to afford the desired product. An exemplary aryl fluoride displacement is provided in Scheme 3, where (R)-3-((4-(2,6-diazaspiro[3.3]heptan-2-yl)phenyl)amino)-5-(3-(3-methyl-2-oxoimidazolidin-1-yl)piperidin-1-yl)pyrazine-2-carboxamide is reacted as described above with 2-(2,6-dioxopiperidin-3-yl)-5-fluoroisoindoline-1,3-dione to provide 3-((4-(6-(2-(2,6-dioxopiperidin-3-yl)-1,3-dioxoisoindolin-5-yl)-2,6-diazaspiro[3.3]heptan-2-yl)phenyl)amino)-5-((R)-3-(3-methyl-2-oxoimidazolidin-1-yl)piperidin-1-yl)pyrazine-2-carboxamide (Compound 34).

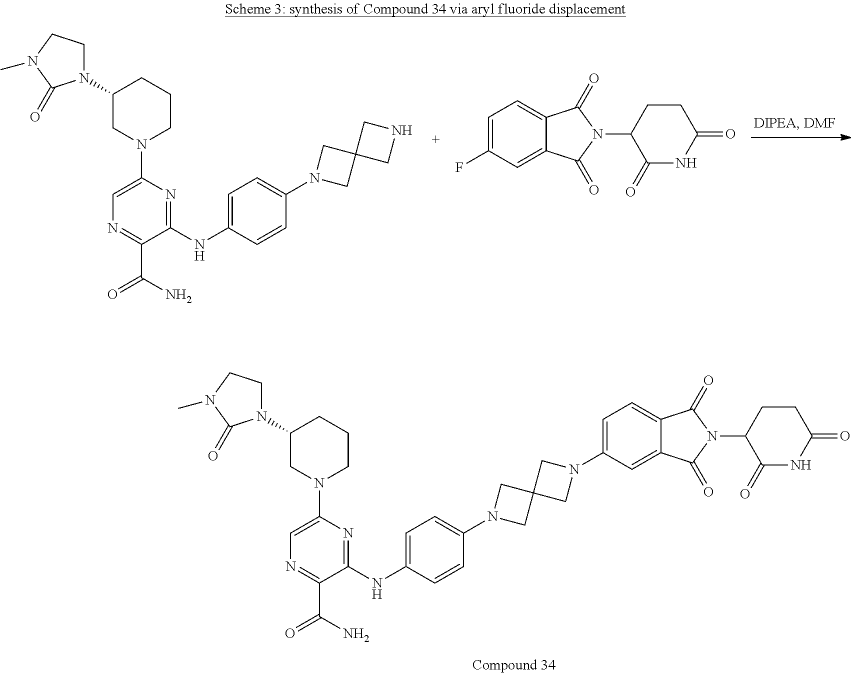 [SMILES: CN1CCN([C@@H]2CCCN(c3cnc(C(N)=O)c(Nc4ccc(N5CC6(CNC6)C5)cc4)n3)C2)C1=O ; O=C1CCC(N2C(=O)c3ccc(F)cc3C2=O)C(=O)N1 ; CN1CCN([C@@H]2CCCN(c3cnc(C(N)=O)c(Nc4ccc(N5CC6(C5)CN(c5ccc7c(c5)C(=O)N(C5CCC(=O)NC5=O)C7=O)C6)cc4)n3)C2)C1=O]

Other aryl amine containing compounds of the invention synthesized by using general procedure 3 are Compounds 55, 29, 47, 53, 58, 64-66, 37, and 30.

The abovementioned synthetic schemes were used to synthesize the compounds in Table 1.

TABLE 1
Example compounds of the present invention.

| Compound | |
|---|---|
| Number | Structure |
| 1 | 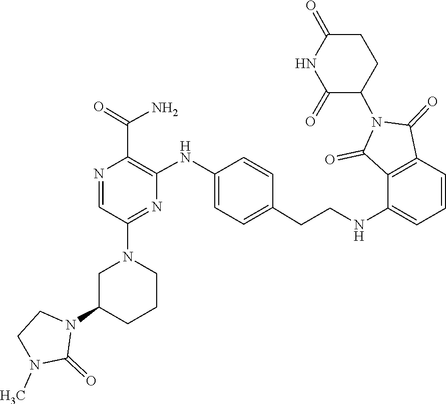 [SMILES: CN1CCN([C@@H]2CCCN(c3cnc(C(N)=O)c(Nc4ccc(CCNc5cccc6c5C(=O)N(C5CCC(=O)NC5=O)C6=O)cc4)n3)C2)C1=O] |
| 2 | 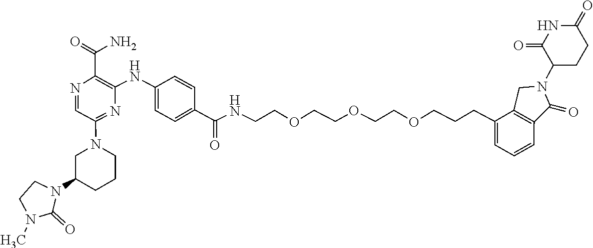 [SMILES: CN1CCN([C@@H]2CCCN(c3cnc(C(N)=O)c(Nc4ccc(C(=O)NCCOCCOCCOCCCc5cccc6c5CN(C5CCC(=O)NC5=O)C6=O)cc4)n3)C2)C1=O] |
| 3 | 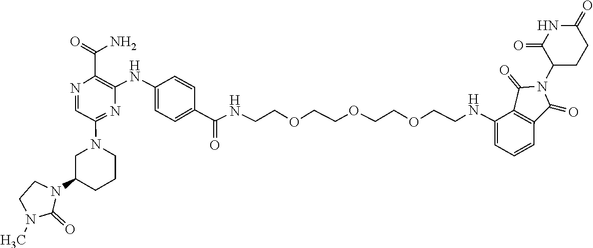 [SMILES: CN1CCN([C@@H]2CCCN(c3cnc(C(N)=O)c(Nc4ccc(C(=O)NCCOCCOCCOCCNc5cccc6c5C(=O)N(C5CCC(=O)NC5=O)C6=O)cc4)n3)C2)C1=O] |
| 4 | 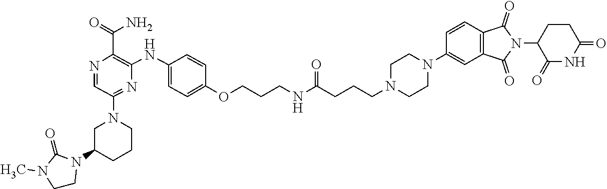 [SMILES: CN1CCN([C@@H]2CCCN(c3cnc(C(N)=O)c(Nc4ccc(OCCCNC(=O)CCCN5CCN(c6ccc7c(c6)C(=O)N(C6CCC(=O)NC6=O)C7=O)CC5)cc4)n3)C2)C1=O] |
| 5 | 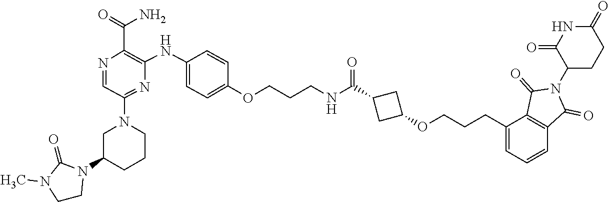 [SMILES: CN1CCN([C@@H]2CCCN(c3cnc(C(N)=O)c(Nc4ccc(OCCCNC(=O)[C@H]5C[C@@H](OCCCc6cccc7c6C(=O)N(C6CCC(=O)NC6=O)C7=O)C5)cc4)n3)C2)C1=O] |
| 6 | 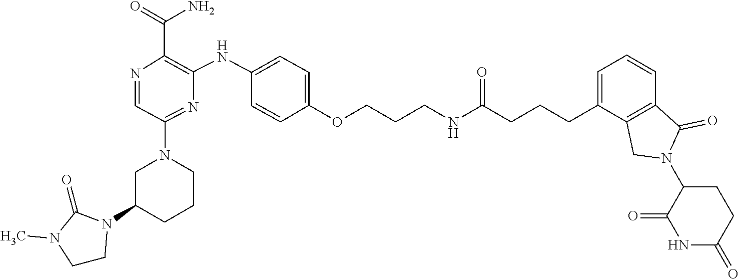 [SMILES: CN1CCN([C@@H]2CCCN(c3cnc(C(N)=O)c(Nc4ccc(OCCCNC(=O)CCCc5cccc6c5CN(C5CCC(=O)NC5=O)C6=O)cc4)n3)C2)C1=O] |
| 7 | 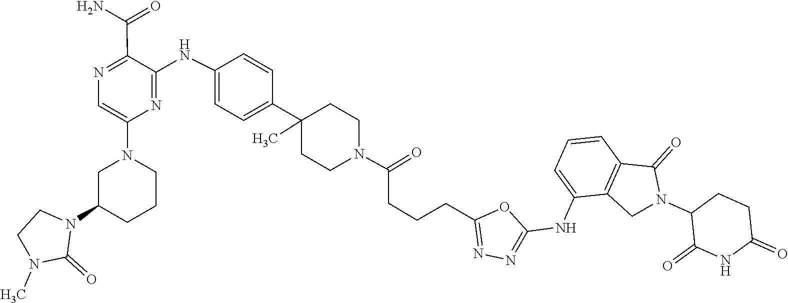 [SMILES: CN1CCN([C@@H]2CCCN(c3cnc(C(N)=O)c(Nc4ccc(C5(C)CCN(C(=O)CCCc6nnc(Nc7cccc8c7CN(C7CCC(=O)NC7=O)C8=O)o6)CC5)cc4)n3)C2)C1=O] |
| 8 | 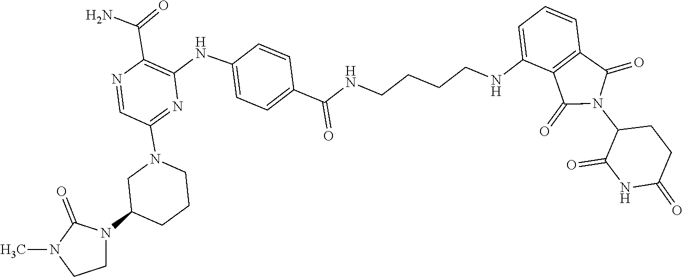 [SMILES: CN1CCN([C@@H]2CCCN(c3cnc(C(N)=O)c(Nc4ccc(C(=O)NCCCCNc5cccc6c5C(=O)N(C5CCC(=O)NC5=O)C6=O)cc4)n3)C2)C1=O] |
| 9 | 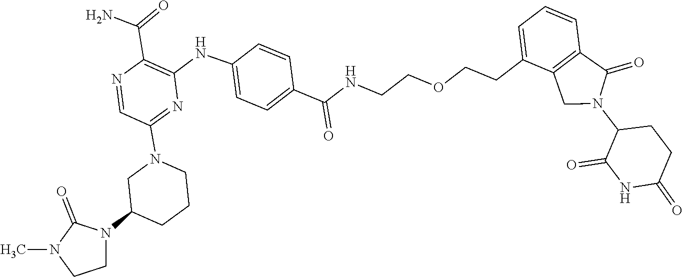 [SMILES: CN1CCN([C@@H]2CCCN(c3cnc(C(N)=O)c(Nc4ccc(C(=O)NCCOCCc5cccc6c5CN(C5CCC(=O)NC5=O)C6=O)cc4)n3)C2)C1=O] |
| 10 | 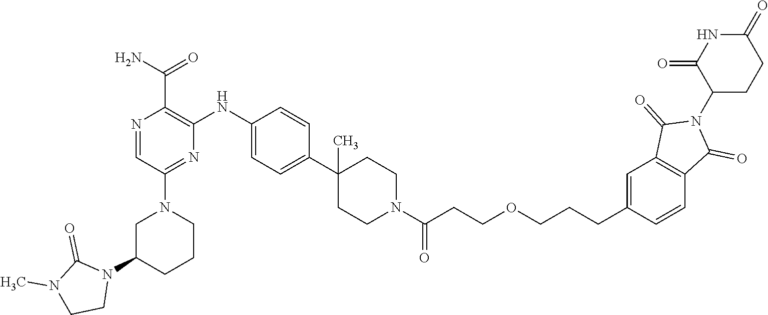 [SMILES: CN1CCN([C@@H]2CCCN(c3cnc(C(N)=O)c(Nc4ccc(C5(C)CCN(C(=O)CCOCCCc6ccc7c(c6)C(=O)N(C6CCC(=O)NC6=O)C7=O)CC5)cc4)n3)C2)C1=O] |
| 11 | 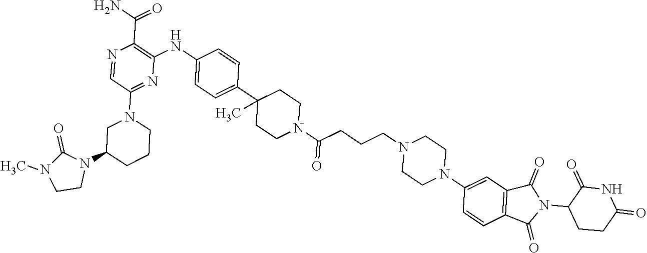 [SMILES: CN1CCN([C@@H]2CCCN(c3cnc(C(N)=O)c(Nc4ccc(C5(C)CCN(C(=O)CCCN6CCN(c7ccc8c(c7)C(=O)N(C7CCC(=O)NC7=O)C8=O)CC6)CC5)cc4)n3)C2)C1=O] |
| 12 | 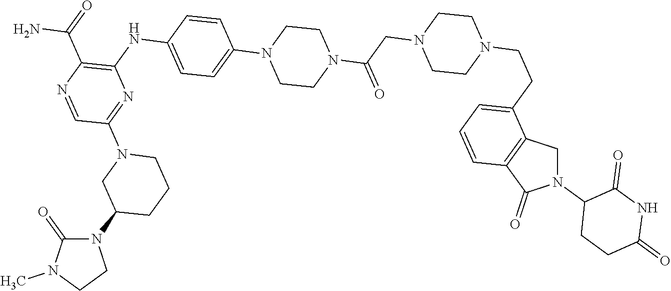 [SMILES: CN1CCN([C@@H]2CCCN(c3cnc(C(N)=O)c(Nc4ccc(N5CCN(C(=O)CN6CCN(CCc7cccc8c7CN(C7CCC(=O)NC7=O)C8=O)CC6)CC5)cc4)n3)C2)C1=O] |
| 13 | 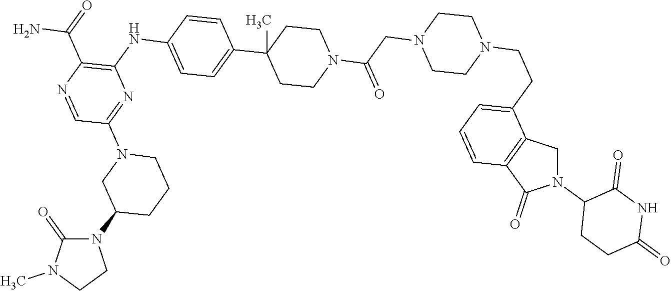 [SMILES: CN1CCN([C@@H]2CCCN(c3cnc(C(N)=O)c(Nc4ccc(C5(C)CCN(C(=O)CN6CCN(CCc7cccc8c7CN(C7CCC(=O)NC7=O)C8=O)CC6)CC5)cc4)n3)C2)C1=O] |
| 14 | 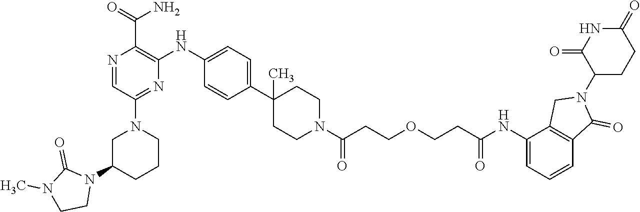 [SMILES: CN1CCN([C@@H]2CCCN(c3cnc(C(N)=O)c(Nc4ccc(C5(C)CCN(C(=O)CCOCCC(=O)Nc6cccc7c6CN(C6CCC(=O)NC6=O)C7=O)CC5)cc4)n3)C2)C1=O] |
| 15 | 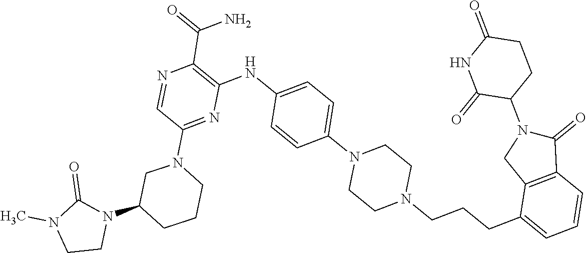 [SMILES: CN1CCN([C@@H]2CCCN(c3cnc(C(N)=O)c(Nc4ccc(N5CCN(CCCc6cccc7c6CN(C6CCC(=O)NC6=O)C7=O)CC5)cc4)n3)C2)C1=O] |
| 16 | 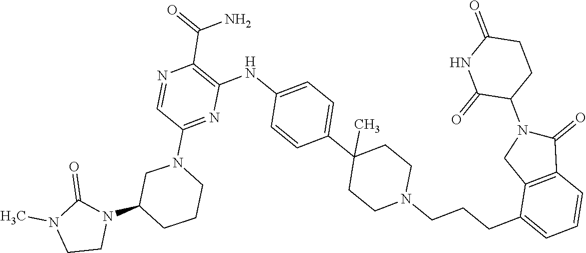 [SMILES: CN1CCN([C@@H]2CCCN(c3cnc(C(N)=O)c(Nc4ccc(C5(C)CCN(CCCc6cccc7c6CN(C6CCC(=O)NC6=O)C7=O)CC5)cc4)n3)C2)C1=O] |
| 17 | 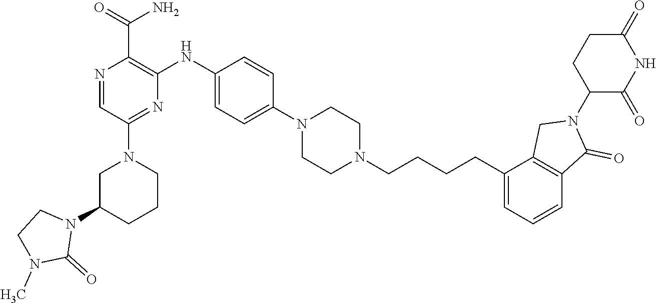 [SMILES: CN1CCN([C@@H]2CCCN(c3cnc(C(N)=O)c(Nc4ccc(N5CCN(CCCCc6cccc7c6CN(C6CCC(=O)NC6=O)C7=O)CC5)cc4)n3)C2)C1=O] |
| 18 | 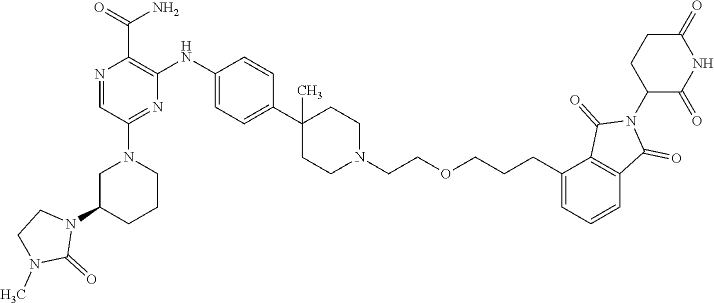 [SMILES: CN1CCN([C@@H]2CCCN(c3cnc(C(N)=O)c(Nc4ccc(C5(C)CCN(CCOCCCc6cccc7c6C(=O)N(C6CCC(=O)NC6=O)C7=O)CC5)cc4)n3)C2)C1=O] |
| 19 | 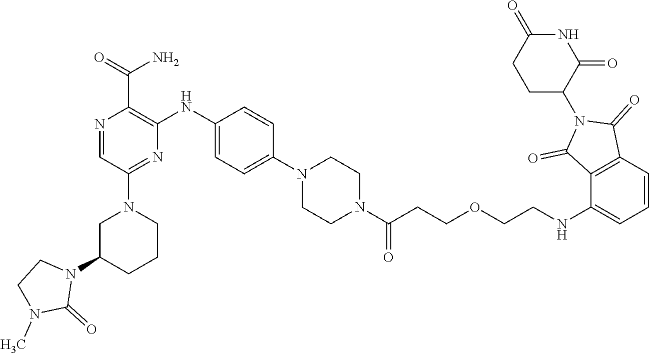 [SMILES: CN1CCN([C@@H]2CCCN(c3cnc(C(N)=O)c(Nc4ccc(N5CCN(C(=O)CCOCCNc6cccc7c6C(=O)N(C6CCC(=O)NC6=O)C7=O)CC5)cc4)n3)C2)C1=O] |
| 20 | 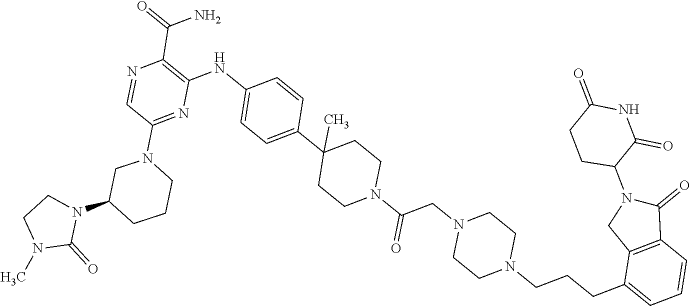 [SMILES: CN1CCN([C@@H]2CCCN(c3cnc(C(N)=O)c(Nc4ccc(C5(C)CCN(C(=O)CN6CCN(CCCc7cccc8c7CN(C7CCC(=O)NC7=O)C8=O)CC6)CC5)cc4)n3)C2)C1=O] |
| 21 | 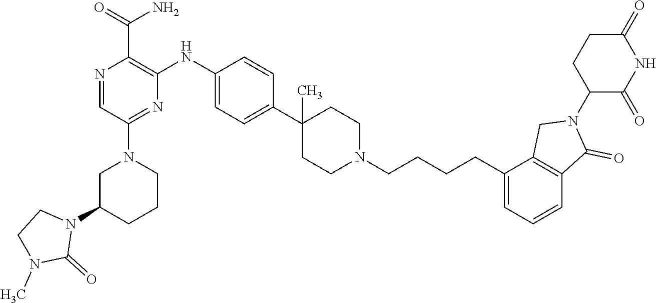 [SMILES: CN1CCN([C@@H]2CCCN(c3cnc(C(N)=O)c(Nc4ccc(C5(C)CCN(CCCCc6cccc7c6CN(C6CCC(=O)NC6=O)C7=O)CC5)cc4)n3)C2)C1=O] |
| 22 | 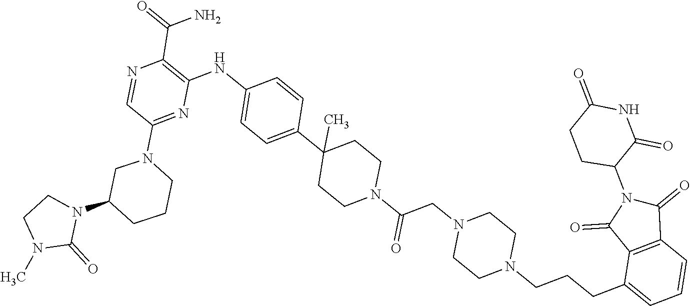 [SMILES: CN1CCN([C@@H]2CCCN(c3cnc(C(N)=O)c(Nc4ccc(C5(C)CCN(C(=O)CN6CCN(CCCc7cccc8c7C(=O)N(C7CCC(=O)NC7=O)C8=O)CC6)CC5)cc4)n3)C2)C1=O] |
| 23 | 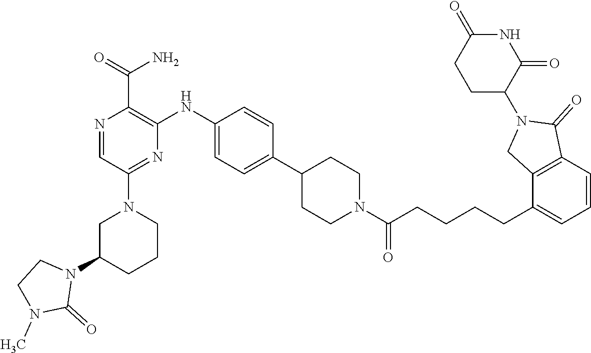 [SMILES: CN1CCN([C@@H]2CCCN(c3cnc(C(N)=O)c(Nc4ccc(C5CCN(C(=O)CCCCc6cccc7c6CN(C6CCC(=O)NC6=O)C7=O)CC5)cc4)n3)C2)C1=O] |
| 24 | 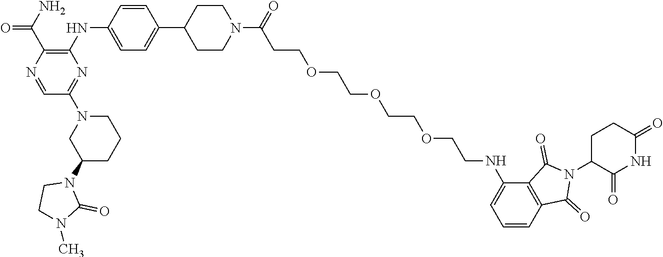 [SMILES: CN1CCN([C@@H]2CCCN(c3cnc(C(N)=O)c(Nc4ccc(C5CCN(C(=O)CCOCCOCCOCCNc6cccc7c6C(=O)N(C6CCC(=O)NC6=O)C7=O)CC5)cc4)n3)C2)C1=O] |
| 25 | 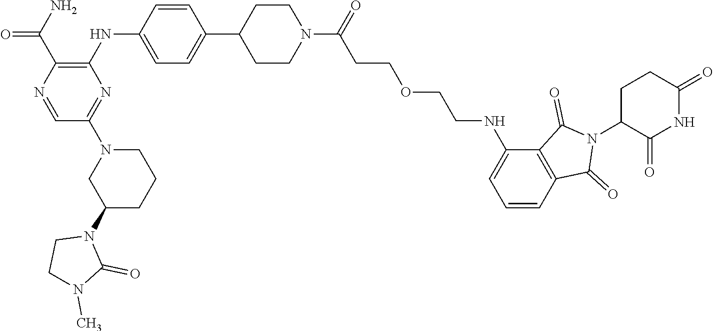 [SMILES: CN1CCN([C@@H]2CCCN(c3cnc(C(N)=O)c(Nc4ccc(C5CCN(C(=O)CCOCCNc6cccc7c6C(=O)N(C6CCC(=O)NC6=O)C7=O)CC5)cc4)n3)C2)C1=O] |
| 26 | 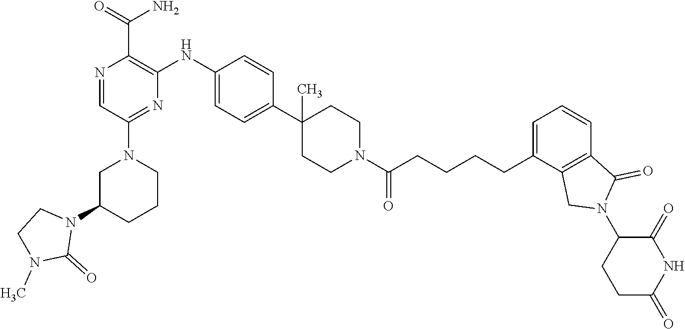 [SMILES: CN1CCN([C@@H]2CCCN(c3cnc(C(N)=O)c(Nc4ccc(C5(C)CCN(C(=O)CCCCc6cccc7c6CN(C6CCC(=O)NC6=O)C7=O)CC5)cc4)n3)C2)C1=O] |
| 27 | 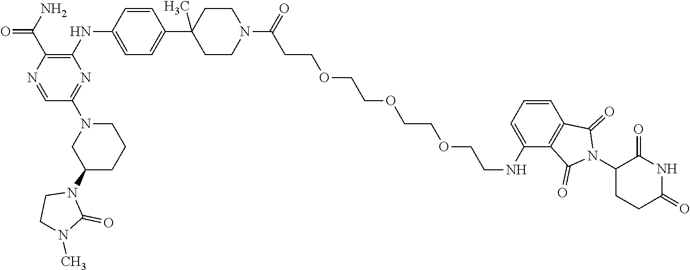 [SMILES: CN1CCN([C@@H]2CCCN(c3cnc(C(N)=O)c(Nc4ccc(C5(C)CCN(C(=O)CCOCCOCCOCCNc6cccc7c6C(=O)N(C6CCC(=O)NC6=O)C7=O)CC5)cc4)n3)C2)C1=O] |
| 28 | 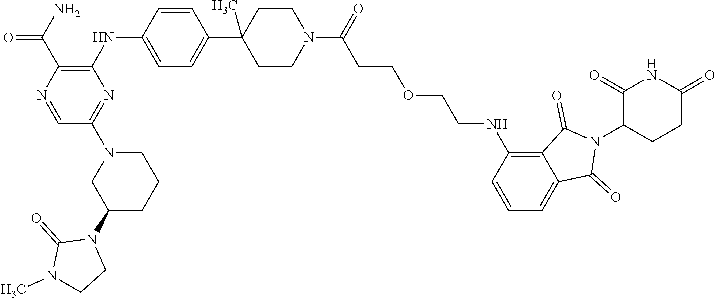 [SMILES: CN1CCN([C@@H]2CCCN(c3cnc(C(N)=O)c(Nc4ccc(C5(C)CCN(C(=O)CCOCCNc6cccc7c6C(=O)N(C6CCC(=O)NC6=O)C7=O)CC5)cc4)n3)C2)C1=O] |
| 29 | 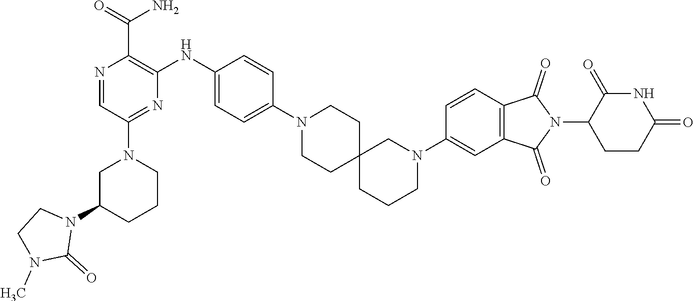 [SMILES: CN1CCN([C@@H]2CCCN(c3cnc(C(N)=O)c(Nc4ccc(N5CCC6(CCCN(c7ccc8c(c7)C(=O)N(C7CCC(=O)NC7=O)C8=O)C6)CC5)cc4)n3)C2)C1=O] |
| 30 | 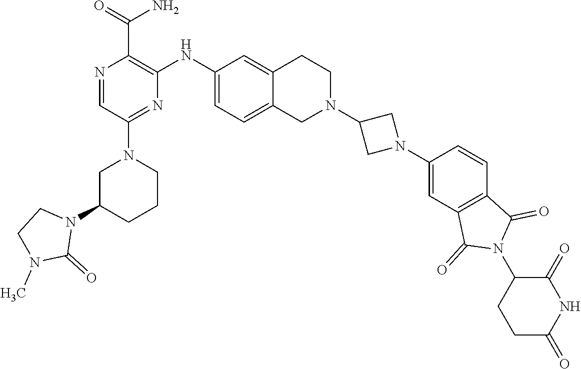 [SMILES: CN1CCN([C@@H]2CCCN(c3cnc(C(N)=O)c(Nc4ccc5c(c4)CCN(C4CN(c6ccc7c(c6)C(=O)N(C6CCC(=O)NC6=O)C7=O)C4)C5)n3)C2)C1=O] |
| 31 | 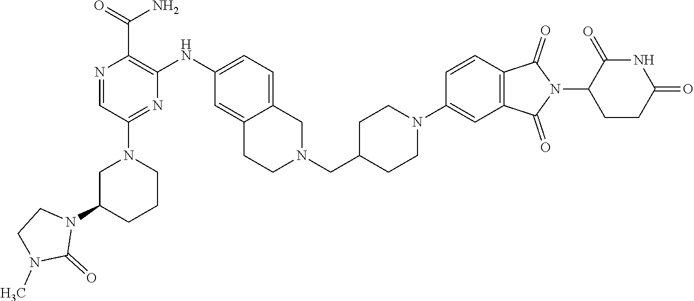 [SMILES: CN1CCN([C@@H]2CCCN(c3cnc(C(N)=O)c(Nc4ccc5c(c4)CCN(CC4CCN(c6ccc7c(c6)C(=O)N(C6CCC(=O)NC6=O)C7=O)CC4)C5)n3)C2)C1=O] |
| 32 | 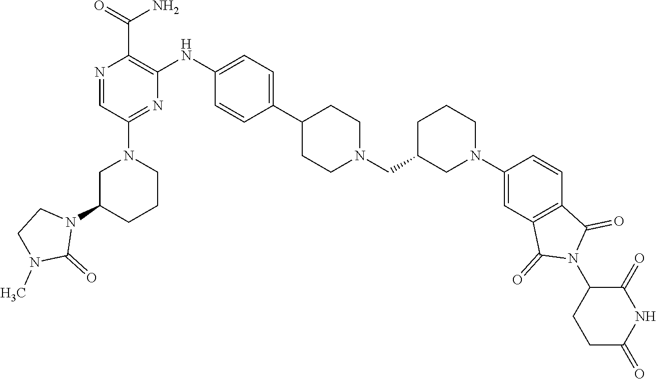 [SMILES: CN1CCN([C@@H]2CCCN(c3cnc(C(N)=O)c(Nc4ccc(C5CCN(C[C@@H]6CCCN(c7ccc8c(c7)C(=O)N(C7CCC(=O)NC7=O)C8=O)C6)CC5)cc4)n3)C2)C1=O] |
| 33 | 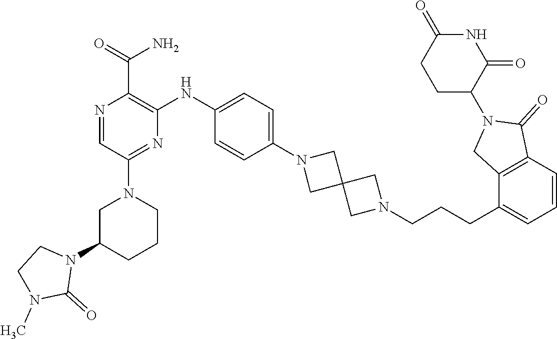 [SMILES: CN1CCN([C@@H]2CCCN(c3cnc(C(N)=O)c(Nc4ccc(N5CC6(CN(CCCc7cccc8c7CN(C7CCC(=O)NC7=O)C8=O)C6)C5)cc4)n3)C2)C1=O] |
| 34 | 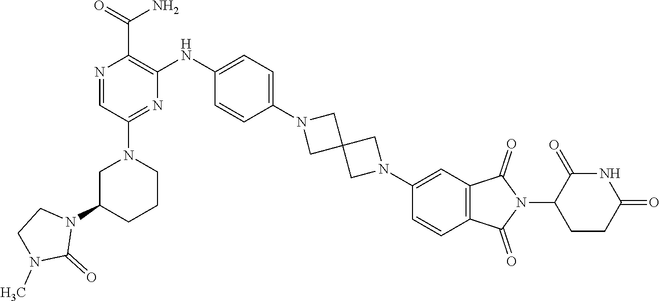 [SMILES: CN1CCN([C@@H]2CCCN(c3cnc(C(N)=O)c(Nc4ccc(N5CC6(C5)CN(c5ccc7c(c5)C(=O)N(C5CCC(=O)NC5=O)C7=O)C6)cc4)n3)C2)C1=O] |
| 35 | 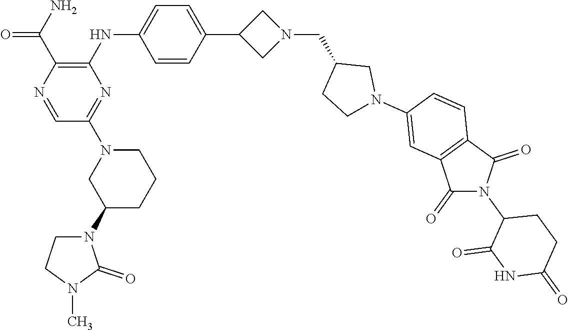 [SMILES: CN1CCN([C@@H]2CCCN(c3cnc(C(N)=O)c(Nc4ccc(C5CN(C[C@H]6CCN(c7ccc8c(c7)C(=O)N(C7CCC(=O)NC7=O)C8=O)C6)C5)cc4)n3)C2)C1=O] |
| 36 | 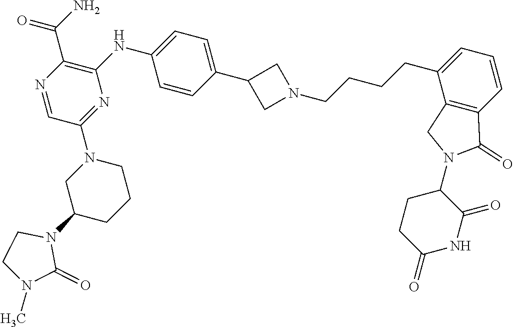 [SMILES: CN1CCN([C@@H]2CCCN(c3cnc(C(N)=O)c(Nc4ccc(C5CN(CCCCc6cccc7c6CN(C6CCC(=O)NC6=O)C7=O)C5)cc4)n3)C2)C1=O] |
| 37 | 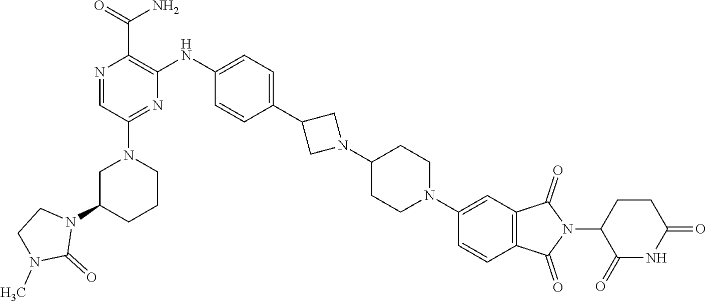 [SMILES: CN1CCN([C@@H]2CCCN(c3cnc(C(N)=O)c(Nc4ccc(C5CN(C6CCN(c7ccc8c(c7)C(=O)N(C7CCC(=O)NC7=O)C8=O)CC6)C5)cc4)n3)C2)C1=O] |
| 38 | 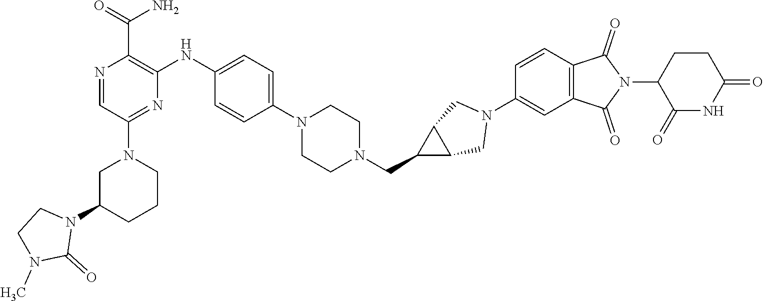 [SMILES: CN1CCN([C@@H]2CCCN(c3cnc(C(N)=O)c(Nc4ccc(N5CCN(C[C@@H]6[C@H]7CN(c8ccc9c(c8)C(=O)N(C8CCC(=O)NC8=O)C9=O)C[C@@H]67)CC5)cc4)n3)C2)C1=O] |
| 39 | 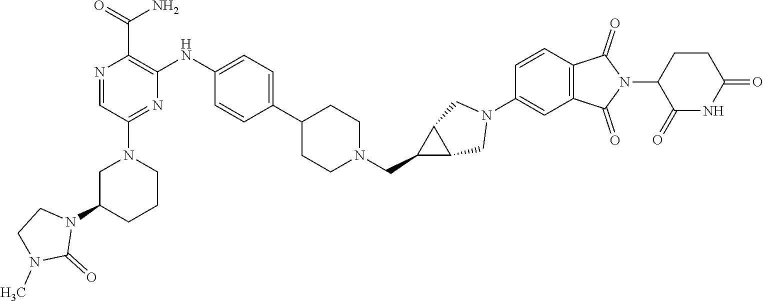 [SMILES: CN1CCN([C@@H]2CCCN(c3cnc(C(N)=O)c(Nc4ccc(C5CCN(C[C@@H]6[C@H]7CN(c8ccc9c(c8)C(=O)N(C8CCC(=O)NC8=O)C9=O)C[C@@H]67)CC5)cc4)n3)C2)C1=O] |
| 40 | 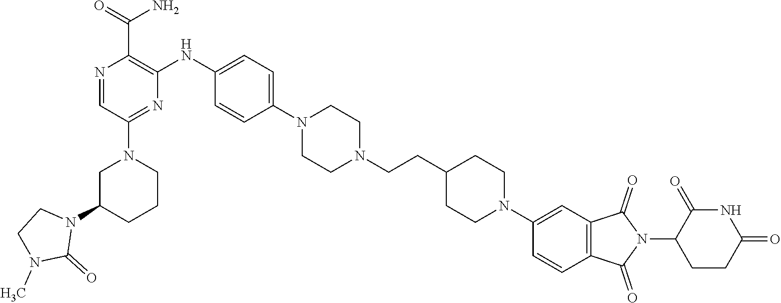 [SMILES: CN1CCN([C@@H]2CCCN(c3cnc(C(N)=O)c(Nc4ccc(N5CCN(CCC6CCN(c7ccc8c(c7)C(=O)N(C7CCC(=O)NC7=O)C8=O)CC6)CC5)cc4)n3)C2)C1=O] |
| 41 | 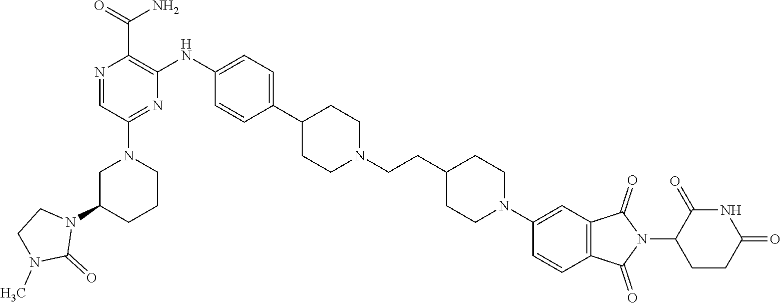 [SMILES: CN1CCN([C@@H]2CCCN(c3cnc(C(N)=O)c(Nc4ccc(C5CCN(CCC6CCN(c7ccc8c(c7)C(=O)N(C7CCC(=O)NC7=O)C8=O)CC6)CC5)cc4)n3)C2)C1=O] |
| 42 | 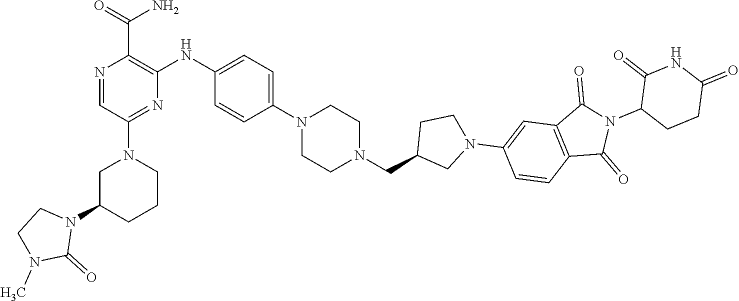 [SMILES: CN1CCN([C@@H]2CCCN(c3cnc(C(N)=O)c(Nc4ccc(N5CCN(C[C@H]6CCN(c7ccc8c(c7)C(=O)N(C7CCC(=O)NC7=O)C8=O)C6)CC5)cc4)n3)C2)C1=O] |
| 43 | 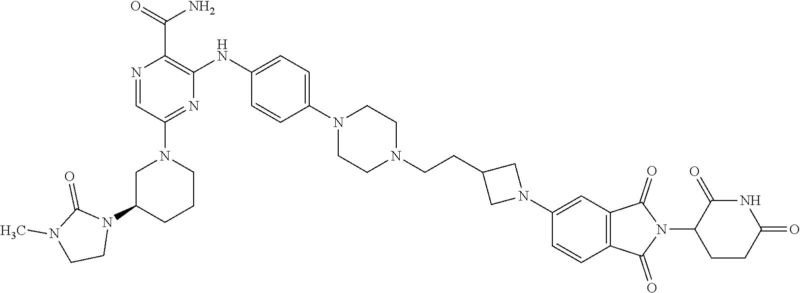 [SMILES: CN1CCN([C@@H]2CCCN(c3cnc(C(N)=O)c(Nc4ccc(N5CCN(CCC6CN(c7ccc8c(c7)C(=O)N(C7CCC(=O)NC7=O)C8=O)C6)CC5)cc4)n3)C2)C1=O] |
| 44 | 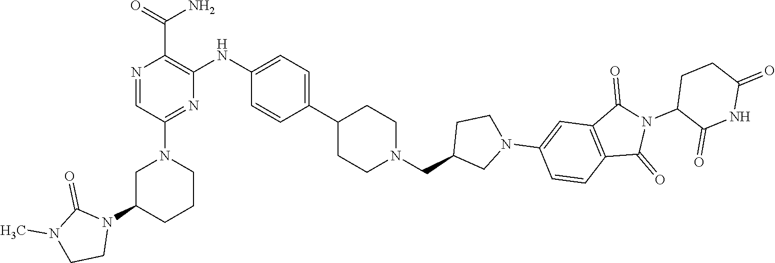 [SMILES: CN1CCN([C@@H]2CCCN(c3cnc(C(N)=O)c(Nc4ccc(C5CCN(C[C@H]6CCN(c7ccc8c(c7)C(=O)N(C7CCC(=O)NC7=O)C8=O)C6)CC5)cc4)n3)C2)C1=O] |
| 45 | 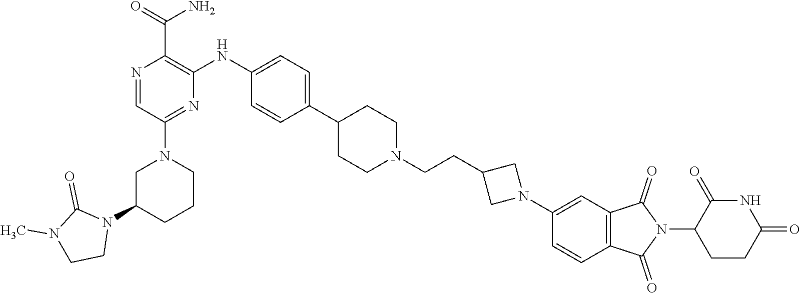 [SMILES: CN1CCN([C@@H]2CCCN(c3cnc(C(N)=O)c(Nc4ccc(C5CCN(CCC6CN(c7ccc8c(c7)C(=O)N(C7CCC(=O)NC7=O)C8=O)C6)CC5)cc4)n3)C2)C1=O] |
| 46 | 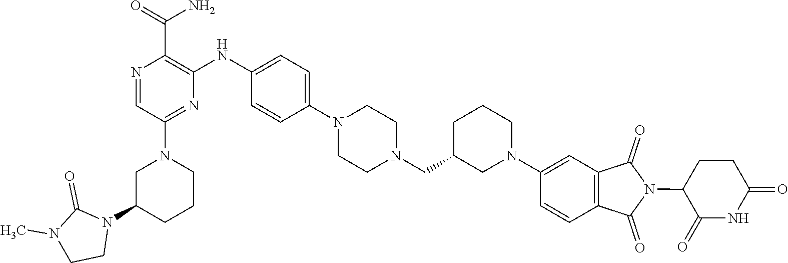 [SMILES: CN1CCN([C@@H]2CCCN(c3cnc(C(N)=O)c(Nc4ccc(N5CCN(C[C@@H]6CCCN(c7ccc8c(c7)C(=O)N(C7CCC(=O)NC7=O)C8=O)C6)CC5)cc4)n3)C2)C1=O] |
| 47 | 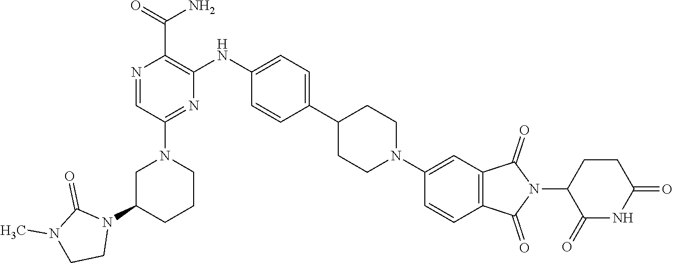 [SMILES: CN1CCN([C@@H]2CCCN(c3cnc(C(N)=O)c(Nc4ccc(C5CCN(c6ccc7c(c6)C(=O)N(C6CCC(=O)NC6=O)C7=O)CC5)cc4)n3)C2)C1=O] |
| 48 | 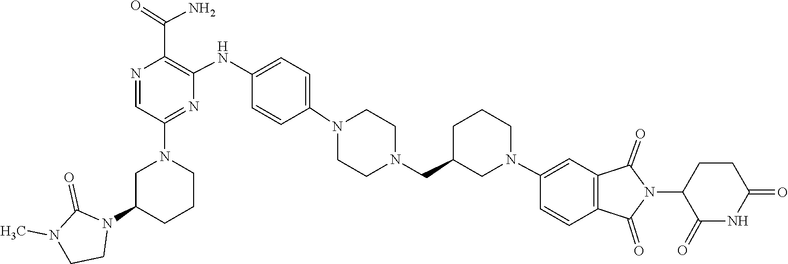 [SMILES: CN1CCN([C@@H]2CCCN(c3cnc(C(N)=O)c(Nc4ccc(N5CCN(C[C@H]6CCCN(c7ccc8c(c7)C(=O)N(C7CCC(=O)NC7=O)C8=O)C6)CC5)cc4)n3)C2)C1=O] |
| 49 | 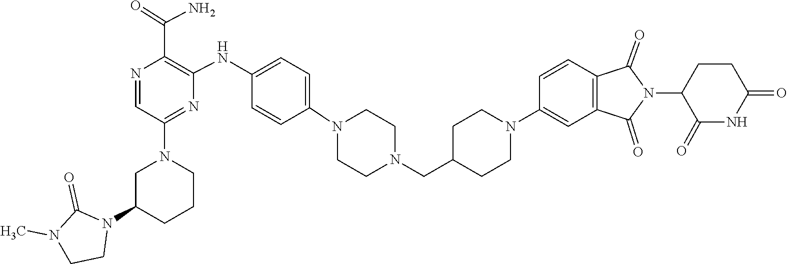 [SMILES: CN1CCN([C@@H]2CCCN(c3cnc(C(N)=O)c(Nc4ccc(N5CCN(CC6CCN(c7ccc8c(c7)C(=O)N(C7CCC(=O)NC7=O)C8=O)CC6)CC5)cc4)n3)C2)C1=O] |
| 50 | 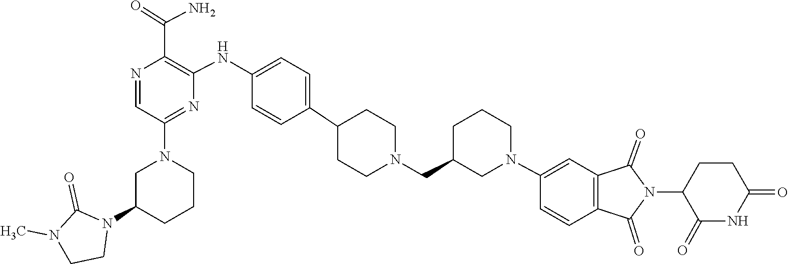 [SMILES: CN1CCN([C@@H]2CCCN(c3cnc(C(N)=O)c(Nc4ccc(C5CCN(C[C@H]6CCCN(c7ccc8c(c7)C(=O)N(C7CCC(=O)NC7=O)C8=O)C6)CC5)cc4)n3)C2)C1=O] |
| 51 | 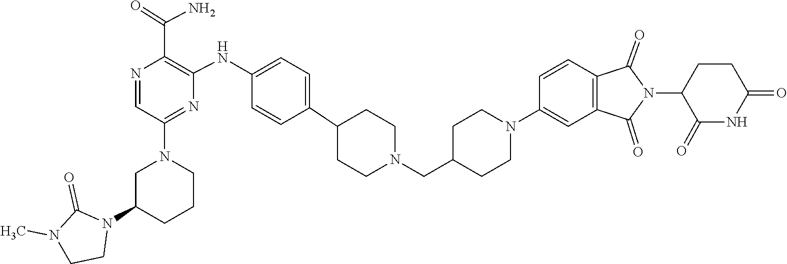 [SMILES: CN1CCN([C@@H]2CCCN(c3cnc(C(N)=O)c(Nc4ccc(C5CCN(CC6CCN(c7ccc8c(c7)C(=O)N(C7CCC(=O)NC7=O)C8=O)CC6)CC5)cc4)n3)C2)C1=O] |
| 52 | 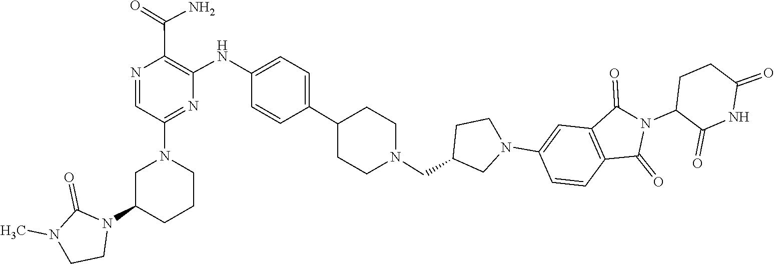 [SMILES: CN1CCN([C@@H]2CCCN(c3cnc(C(N)=O)c(Nc4ccc(C5CCN(C[C@@H]6CCN(c7ccc8c(c7)C(=O)N(C7CCC(=O)NC7=O)C8=O)C6)CC5)cc4)n3)C2)C1=O] |
| 53 | 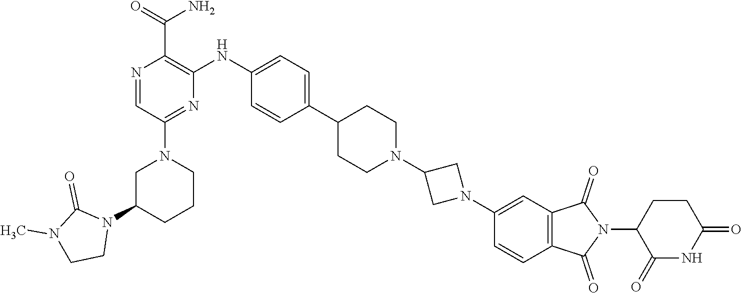 [SMILES: CN1CCN([C@@H]2CCCN(c3cnc(C(N)=O)c(Nc4ccc(C5CCN(C6CN(c7ccc8c(c7)C(=O)N(C7CCC(=O)NC7=O)C8=O)C6)CC5)cc4)n3)C2)C1=O] |
| 54 | 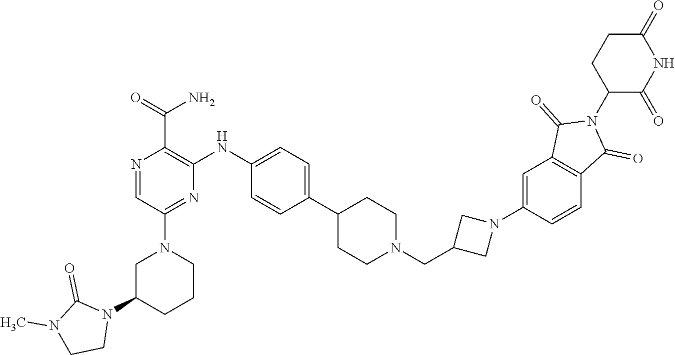 [SMILES: CN1CCN([C@@H]2CCCN(c3cnc(C(N)=O)c(Nc4ccc(C5CCN(CC6CN(c7ccc8c(c7)C(=O)N(C7CCC(=O)NC7=O)C8=O)C6)CC5)cc4)n3)C2)C1=O] |
| 55 | 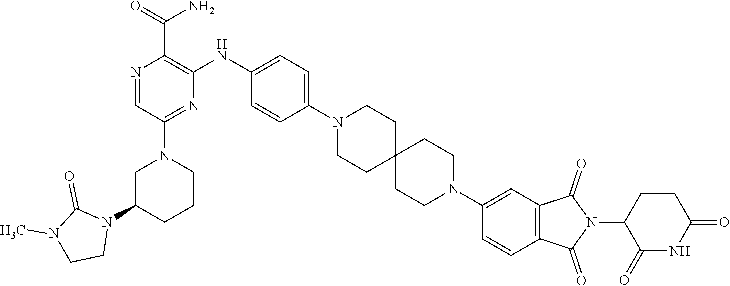 [SMILES: CN1CCN([C@@H]2CCCN(c3cnc(C(N)=O)c(Nc4ccc(N5CCC6(CC5)CCN(c5ccc7c(c5)C(=O)N(C5CCC(=O)NC5=O)C7=O)CC6)cc4)n3)C2)C1=O] |
| 56 | 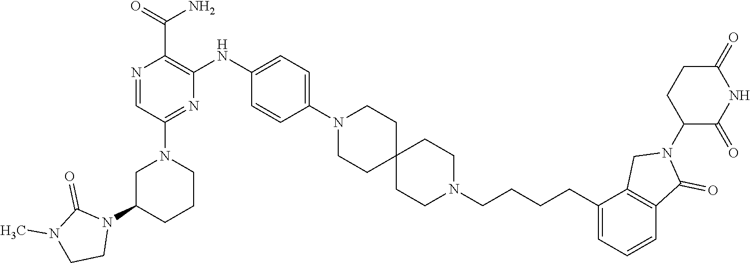 [SMILES: CN1CCN([C@@H]2CCCN(c3cnc(C(N)=O)c(Nc4ccc(N5CCC6(CCN(CCCCc7cccc8c7CN(C7CCC(=O)NC7=O)C8=O)CC6)CC5)cc4)n3)C2)C1=O] |
| 57 | 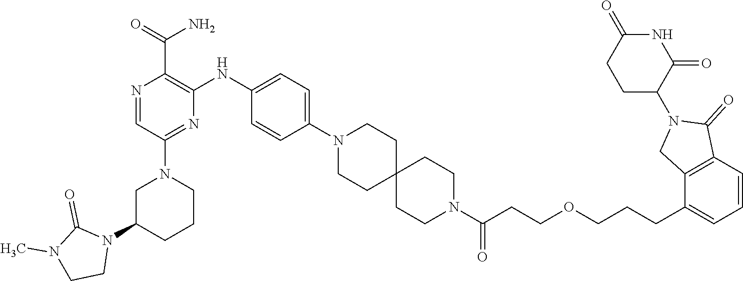 [SMILES: CN1CCN([C@@H]2CCCN(c3cnc(C(N)=O)c(Nc4ccc(N5CCC6(CCN(C(=O)CCOCCCc7cccc8c7CN(C7CCC(=O)NC7=O)C8=O)CC6)CC5)cc4)n3)C2)C1=O] |
| 58 | 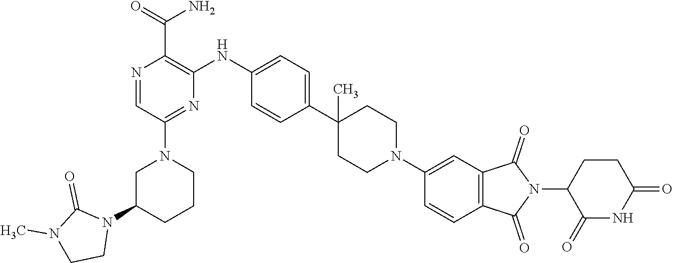 [SMILES: CN1CCN([C@@H]2CCCN(c3cnc(C(N)=O)c(Nc4ccc(C5(C)CCN(c6ccc7c(c6)C(=O)N(C6CCC(=O)NC6=O)C7=O)CC5)cc4)n3)C2)C1=O] |
| 59 | 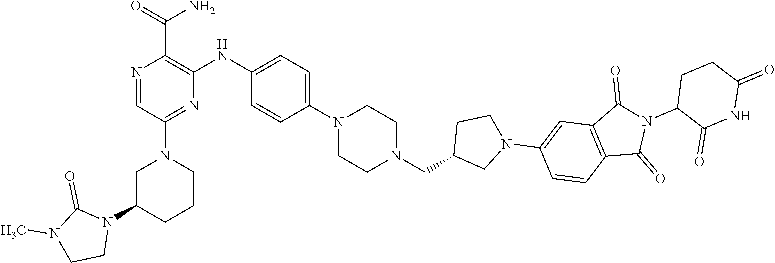 [SMILES: CN1CCN([C@@H]2CCCN(c3cnc(C(N)=O)c(Nc4ccc(N5CCN(C[C@@H]6CCN(c7ccc8c(c7)C(=O)N(C7CCC(=O)NC7=O)C8=O)C6)CC5)cc4)n3)C2)C1=O] |
| 60 | 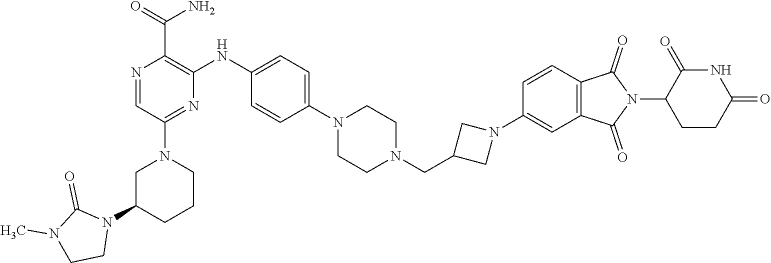 [SMILES: CN1CCN([C@@H]2CCCN(c3cnc(C(N)=O)c(Nc4ccc(N5CCN(CC6CN(c7ccc8c(c7)C(=O)N(C7CCC(=O)NC7=O)C8=O)C6)CC5)cc4)n3)C2)C1=O] |
| 61 | 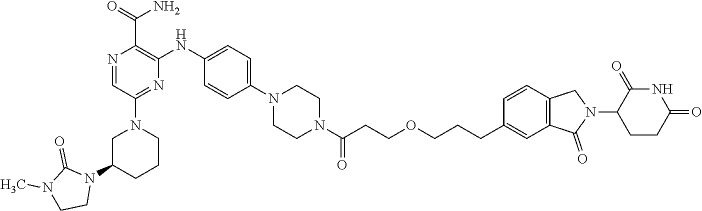 [SMILES: CN1CCN([C@@H]2CCCN(c3cnc(C(N)=O)c(Nc4ccc(N5CCN(C(=O)CCOCCCc6ccc7c(c6)C(=O)N(C6CCC(=O)NC6=O)C7)CC5)cc4)n3)C2)C1=O] |
| 62 | 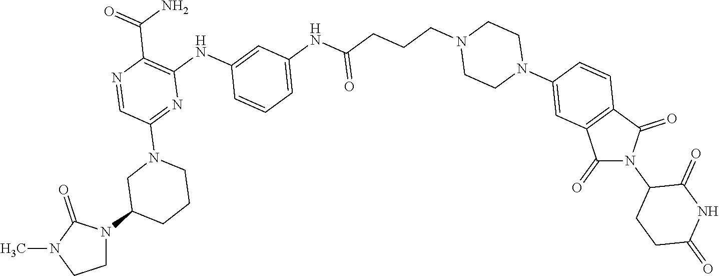 [SMILES: CN1CCN([C@@H]2CCCN(c3cnc(C(N)=O)c(Nc4cccc(NC(=O)CCCN5CCN(c6ccc7c(c6)C(=O)N(C6CCC(=O)NC6=O)C7=O)CC5)c4)n3)C2)C1=O] |
| 63 | 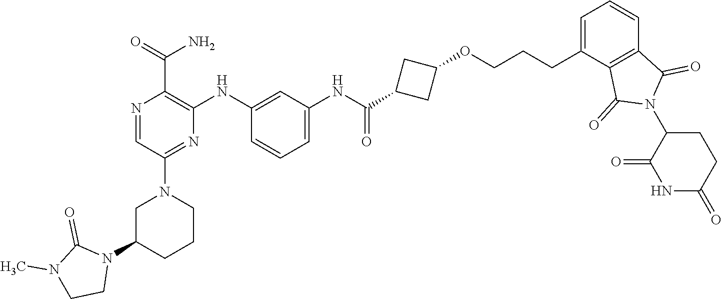 [SMILES: CN1CCN([C@@H]2CCCN(c3cnc(C(N)=O)c(Nc4cccc(NC(=O)[C@H]5C[C@@H](OCCCc6cccc7c6C(=O)N(C6CCC(=O)NC6=O)C7=O)C5)c4)n3)C2)C1=O] |
| 64 | 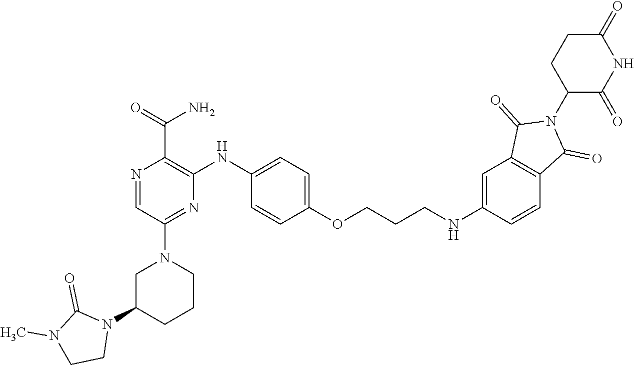 [SMILES: CN1CCN([C@@H]2CCCN(c3cnc(C(N)=O)c(Nc4ccc(OCCCNc5ccc6c(c5)C(=O)N(C5CCC(=O)NC5=O)C6=O)cc4)n3)C2)C1=O] |
| 65 | 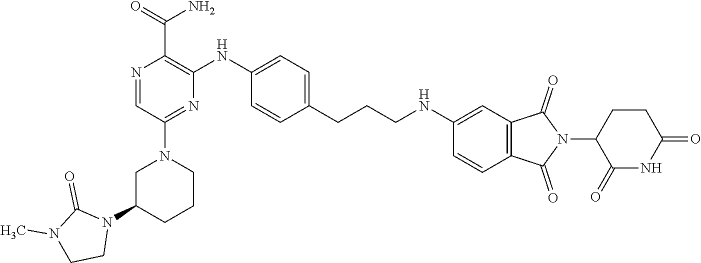 [SMILES: CN1CCN([C@@H]2CCCN(c3cnc(C(N)=O)c(Nc4ccc(CCCNc5ccc6c(c5)C(=O)N(C5CCC(=O)NC5=O)C6=O)cc4)n3)C2)C1=O] |
| 66 | 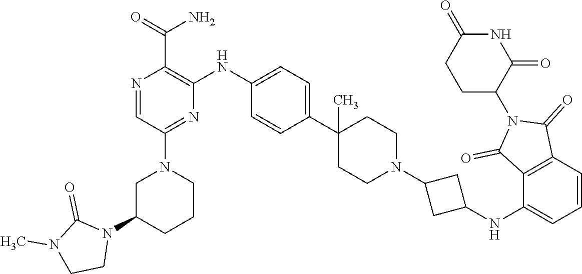 [SMILES: CN1CCN([C@@H]2CCCN(c3cnc(C(N)=O)c(Nc4ccc(C5(C)CCN(C6CC(Nc7cccc8c7C(=O)N(C7CCC(=O)NC7=O)C8=O)C6)CC5)cc4)n3)C2)C1=O] |
| 67 | 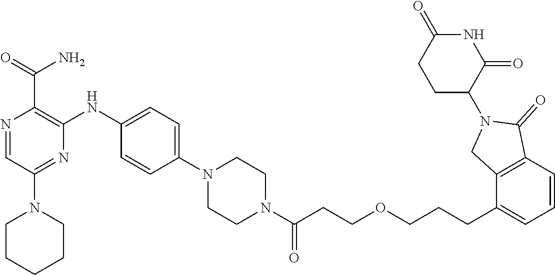 [SMILES: NC(=O)c1ncc(N2CCCCC2)nc1Nc1ccc(N2CCN(C(=O)CCOCCCc3cccc4c3CN(C3CCC(=O)NC3=O)C4=O)CC2)cc1] |
| 68 | 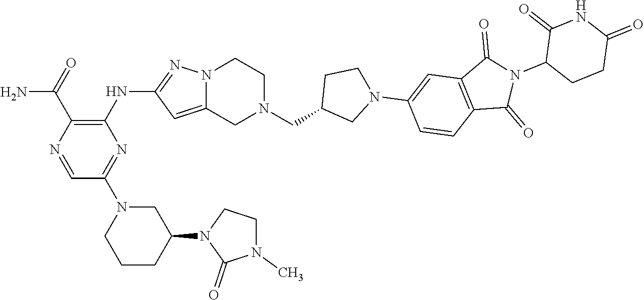 [SMILES: CN1CCN([C@H]2CCCN(c3cnc(C(N)=O)c(Nc4cc5n(n4)CCN(C[C@@H]4CCN(c6ccc7c(c6)C(=O)N(C6CCC(=O)NC6=O)C7=O)C4)C5)n3)C2)C1=O] |
| 69 | 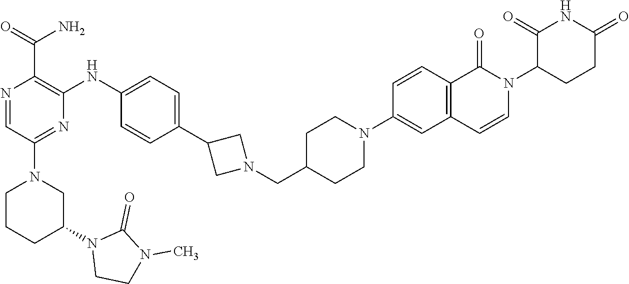 [SMILES: CN1CCN([C@@H]2CCCN(c3cnc(C(N)=O)c(Nc4ccc(C5CN(CC6CCN(c7ccc8c(=O)n(C9CCC(=O)NC9=O)ccc8c7)CC6)C5)cc4)n3)C2)C1=O] |
| 70 | 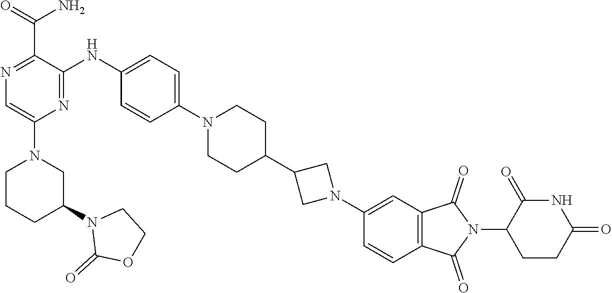 [SMILES: NC(=O)c1ncc(N2CCC[C@H](N3CCOC3=O)C2)nc1Nc1ccc(N2CCC(C3CN(c4ccc5c(c4)C(=O)N(C4CCC(=O)NC4=O)C5=O)C3)CC2)cc1] |
| 71 | 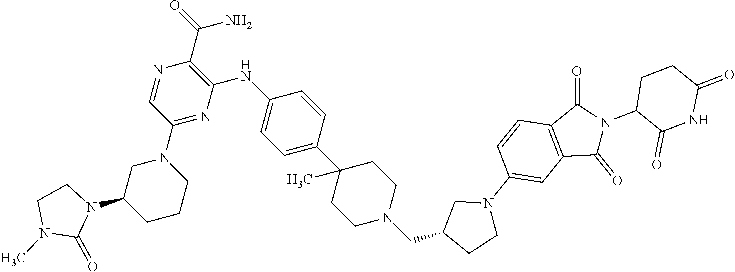 [SMILES: CN1CCN([C@@H]2CCCN(c3cnc(C(N)=O)c(Nc4ccc(C5(C)CCN(C[C@H]6CCN(c7ccc8c(c7)C(=O)N(C7CCC(=O)NC7=O)C8=O)C6)CC5)cc4)n3)C2)C1=O] |
| 72 | 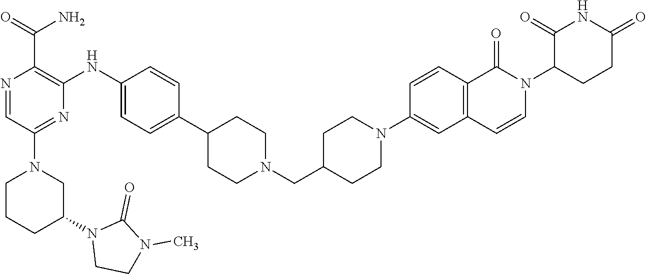 [SMILES: CN1CCN([C@@H]2CCCN(c3cnc(C(N)=O)c(Nc4ccc(C5CCN(CC6CCN(c7ccc8c(=O)n(C9CCC(=O)NC9=O)ccc8c7)CC6)CC5)cc4)n3)C2)C1=O] |
| 73 | 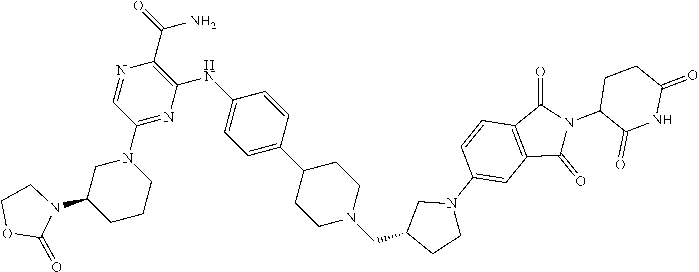 [SMILES: NC(=O)c1ncc(N2CCC[C@@H](N3CCOC3=O)C2)nc1Nc1ccc(C2CCN(C[C@H]3CCN(c4ccc5c(c4)C(=O)N(C4CCC(=O)NC4=O)C5=O)C3)CC2)cc1] |
| 74 | 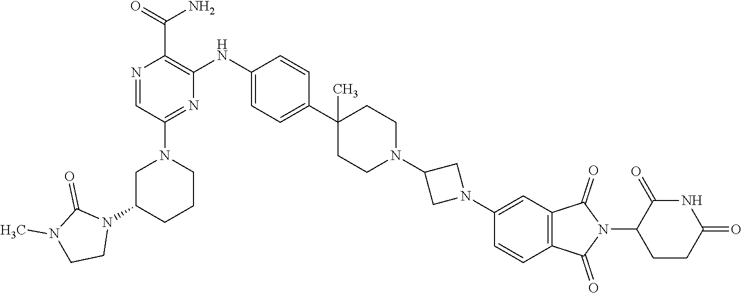 [SMILES: CN1CCN([C@H]2CCCN(c3cnc(C(N)=O)c(Nc4ccc(C5(C)CCN(C6CN(c7ccc8c(c7)C(=O)N(C7CCC(=O)NC7=O)C8=O)C6)CC5)cc4)n3)C2)C1=O] |
| 75 | 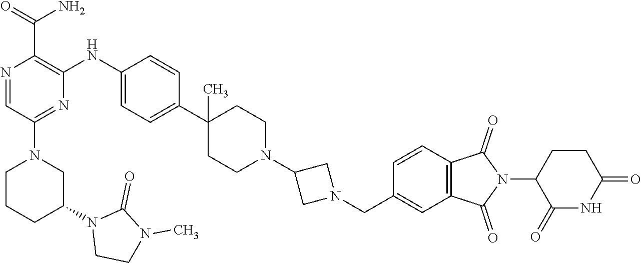 [SMILES: CN1CCN([C@@H]2CCCN(c3cnc(C(N)=O)c(Nc4ccc(C5(C)CCN(C6CN(Cc7ccc8c(c7)C(=O)N(C7CCC(=O)NC7=O)C8=O)C6)CC5)cc4)n3)C2)C1=O] |
| 76 | 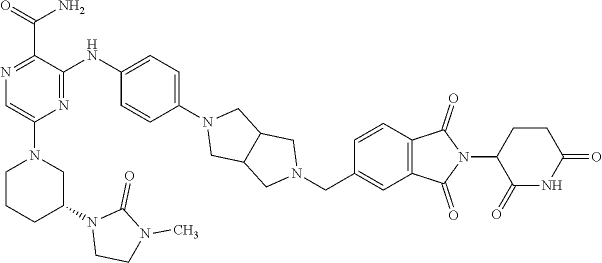 [SMILES: CN1CCN([C@@H]2CCCN(c3cnc(C(N)=O)c(Nc4ccc(N5CC6CN(Cc7ccc8c(c7)C(=O)N(C7CCC(=O)NC7=O)C8=O)CC6C5)cc4)n3)C2)C1=O] |
| 77 | 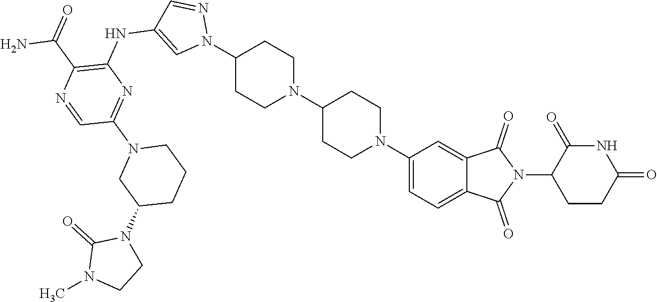 [SMILES: CN1CCN([C@H]2CCCN(c3cnc(C(N)=O)c(Nc4cnn(C5CCN(C6CCN(c7ccc8c(c7)C(=O)N(C7CCC(=O)NC7=O)C8=O)CC6)CC5)c4)n3)C2)C1=O] |
| 78 | 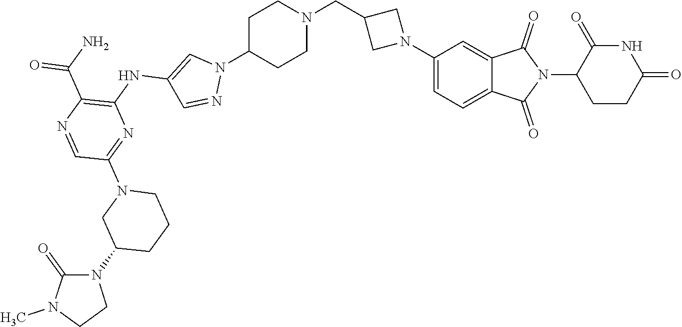 [SMILES: CN1CCN([C@H]2CCCN(c3cnc(C(N)=O)c(Nc4cnn(C5CCN(CC6CN(c7ccc8c(c7)C(=O)N(C7CCC(=O)NC7=O)C8=O)C6)CC5)c4)n3)C2)C1=O] |
| 79 | 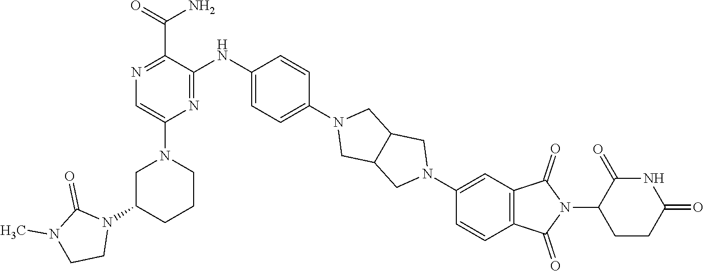 [SMILES: CN1CCN([C@H]2CCCN(c3cnc(C(N)=O)c(Nc4ccc(N5CC6CN(c7ccc8c(c7)C(=O)N(C7CCC(=O)NC7=O)C8=O)CC6C5)cc4)n3)C2)C1=O] |
| 80 | 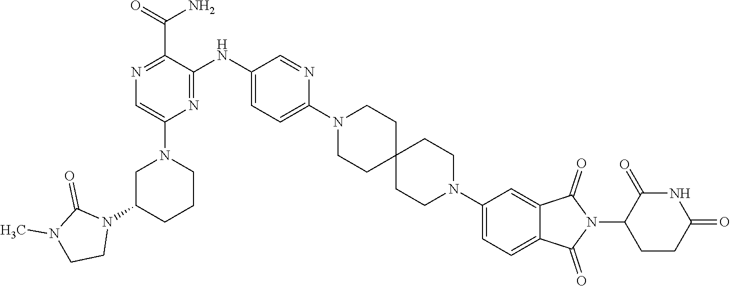 [SMILES: CN1CCN([C@H]2CCCN(c3cnc(C(N)=O)c(Nc4ccc(N5CCC6(CCN(c7ccc8c(c7)C(=O)N(C7CCC(=O)NC7=O)C8=O)CC6)CC5)nc4)n3)C2)C1=O] |
| 81 | 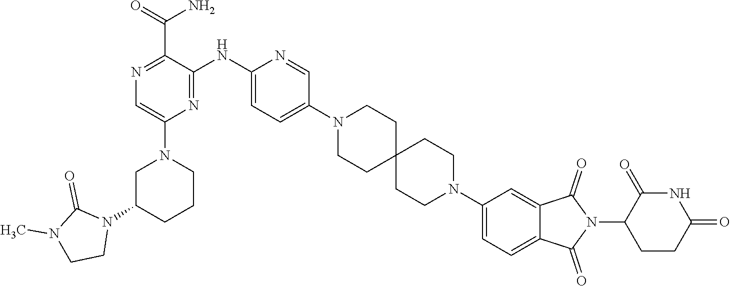 [SMILES: CN1CCN([C@H]2CCCN(c3cnc(C(N)=O)c(Nc4ccc(N5CCC6(CC5)CCN(c5ccc7c(c5)C(=O)N(C5CCC(=O)NC5=O)C7=O)CC6)cn4)n3)C2)C1=O] |
| 82 | 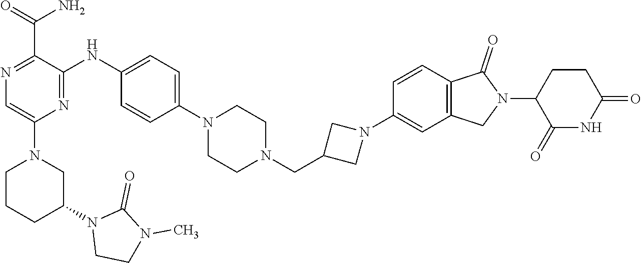 [SMILES: CN1CCN([C@@H]2CCCN(c3cnc(C(N)=O)c(Nc4ccc(N5CCN(CC6CN(c7ccc8c(c7)CN(C7CCC(=O)NC7=O)C8=O)C6)CC5)cc4)n3)C2)C1=O] |
| 83 | 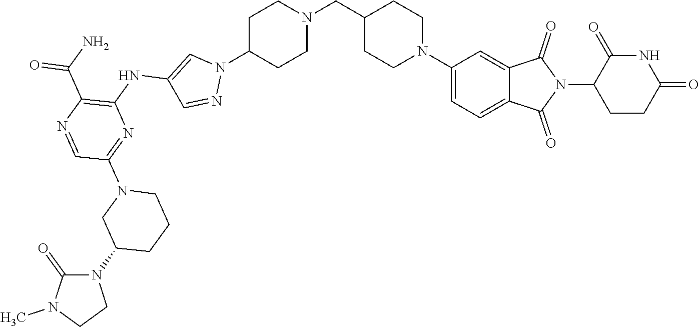 [SMILES: CN1CCN([C@H]2CCCN(c3cnc(C(N)=O)c(Nc4cnn(C5CCN(CC6CCN(c7ccc8c(c7)C(=O)N(C7CCC(=O)NC7=O)C8=O)CC6)CC5)c4)n3)C2)C1=O] |
| 84 | 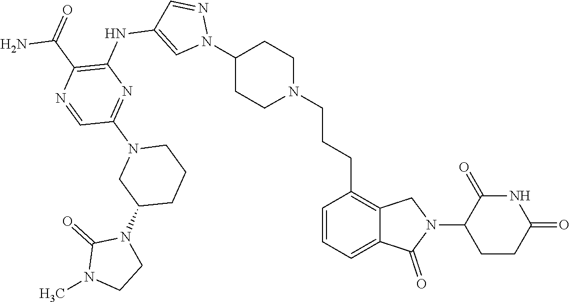 [SMILES: CN1CCN([C@H]2CCCN(c3cnc(C(N)=O)c(Nc4cnn(C5CCN(CCCc6cccc7c6CN(C6CCC(=O)NC6=O)C7=O)CC5)c4)n3)C2)C1=O] |
| 85 | 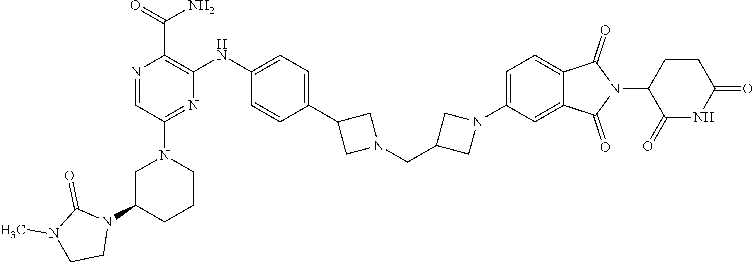 [SMILES: CN1CCN([C@@H]2CCCN(c3cnc(C(N)=O)c(Nc4ccc(C5CN(CC6CN(c7ccc8c(c7)C(=O)N(C7CCC(=O)NC7=O)C8=O)C6)C5)cc4)n3)C2)C1=O] |
| 86 | 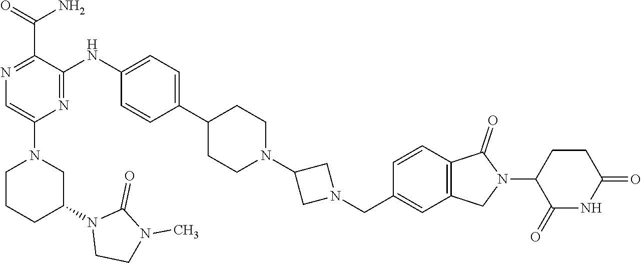 [SMILES: CN1CCN([C@@H]2CCCN(c3cnc(C(N)=O)c(Nc4ccc(C5CCN(C6CN(Cc7ccc8c(c7)CN(C7CCC(=O)NC7=O)C8=O)C6)CC5)cc4)n3)C2)C1=O] |
| 87 | 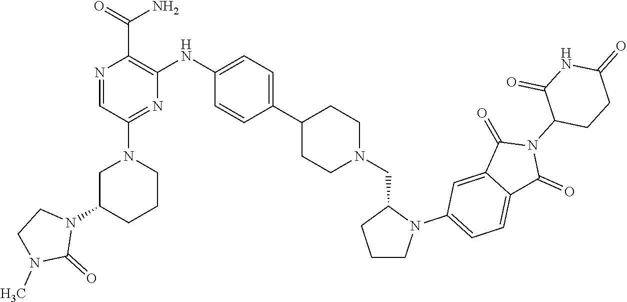 [SMILES: CN1CCN([C@H]2CCCN(c3cnc(C(N)=O)c(Nc4ccc(C5CCN(C[C@H]6CCCN6c6ccc7c(c6)C(=O)N(C6CCC(=O)NC6=O)C7=O)CC5)cc4)n3)C2)C1=O] |
| 88 | 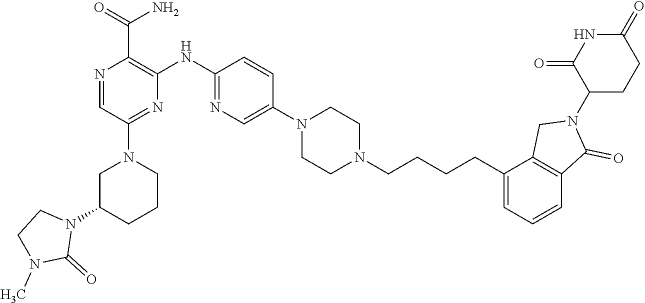 [SMILES: CN1CCN([C@H]2CCCN(c3cnc(C(N)=O)c(Nc4ccc(N5CCN(CCCCc6cccc7c6CN(C6CCC(=O)NC6=O)C7=O)CC5)cn4)n3)C2)C1=O] |
| 89 | 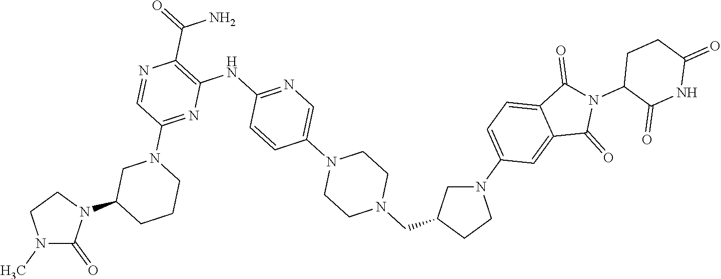 [SMILES: CN1CCN([C@@H]2CCCN(c3cnc(C(N)=O)c(Nc4ccc(N5CCN(C[C@H]6CCN(c7ccc8c(c7)C(=O)N(C7CCC(=O)NC7=O)C8=O)C6)CC5)cn4)n3)C2)C1=O] |
| 90 | 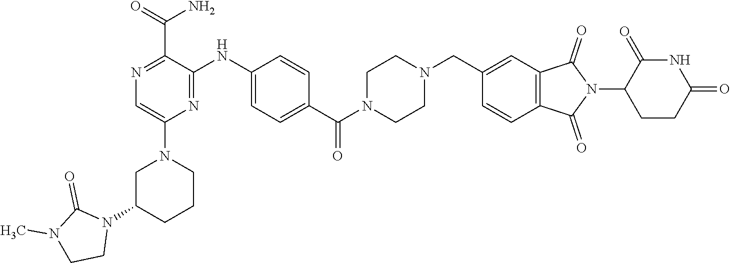 [SMILES: CN1CCN([C@H]2CCCN(c3cnc(C(N)=O)c(Nc4ccc(C(=O)N5CCN(Cc6ccc7c(c6)C(=O)N(C6CCC(=O)NC6=O)C7=O)CC5)cc4)n3)C2)C1=O] |
| 91 | 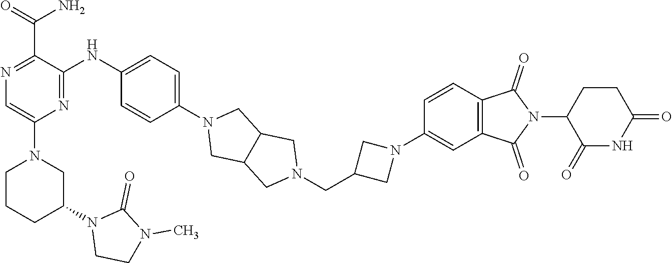 [SMILES: CN1CCN([C@@H]2CCCN(c3cnc(C(N)=O)c(Nc4ccc(N5CC6CN(CC7CN(c8ccc9c(c8)C(=O)N(C8CCC(=O)NC8=O)C9=O)C7)CC6C5)cc4)n3)C2)C1=O] |
| 92 | 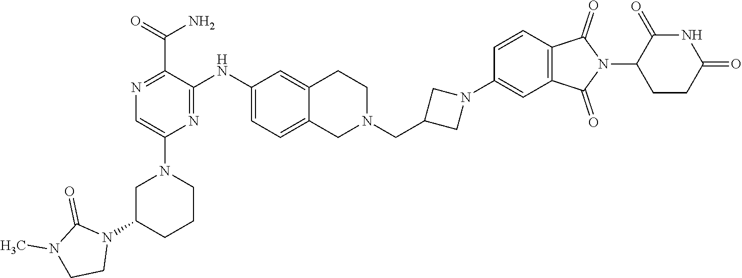 [SMILES: CN1CCN([C@H]2CCCN(c3cnc(C(N)=O)c(Nc4ccc5c(c4)CCN(CC4CN(c6ccc7c(c6)C(=O)N(C6CCC(=O)NC6=O)C7=O)C4)C5)n3)C2)C1=O] |
| 93 | 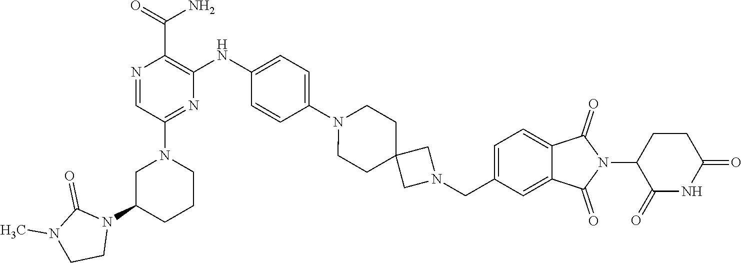 [SMILES: CN1CCN([C@@H]2CCCN(c3cnc(C(N)=O)c(Nc4ccc(N5CCC6(CC5)CN(Cc5ccc7c(c5)C(=O)N(C5CCC(=O)NC5=O)C7=O)C6)cc4)n3)C2)C1=O] |
| 94 | 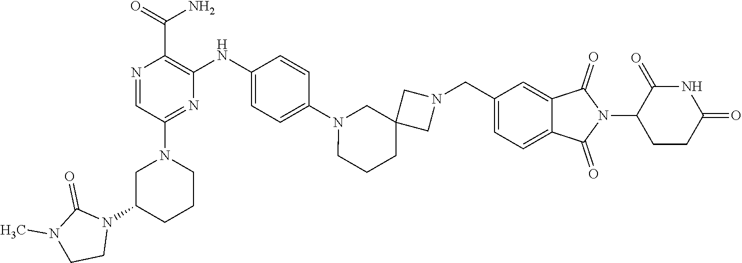 [SMILES: CN1CCN([C@H]2CCCN(c3cnc(C(N)=O)c(Nc4ccc(N5CCCC6(CN(Cc7ccc8c(c7)C(=O)N(C7CCC(=O)NC7=O)C8=O)C6)C5)cc4)n3)C2)C1=O] |
| 95 | 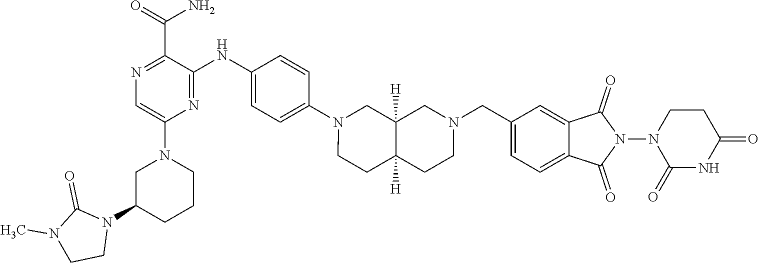 [SMILES: CN1CCN([C@@H]2CCCN(c3cnc(C(N)=O)c(Nc4ccc(N5CC[C@@H]6CCN(Cc7ccc8c(c7)C(=O)N(N7CCC(=O)NC7=O)C8=O)C[C@@H]6C5)cc4)n3)C2)C1=O] |
| 96 | 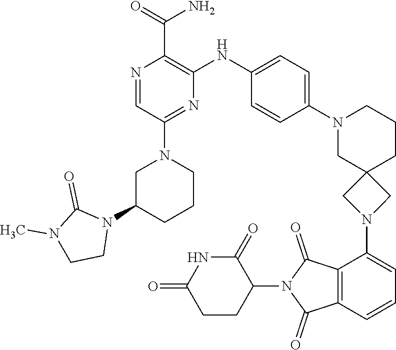 [SMILES: CN1CCN([C@@H]2CCCN(c3cnc(C(N)=O)c(Nc4ccc(N5CCCC6(C5)CN(c5cccc7c5C(=O)N(C5CCC(=O)NC5=O)C7=O)C6)cc4)n3)C2)C1=O] |
| 97 | 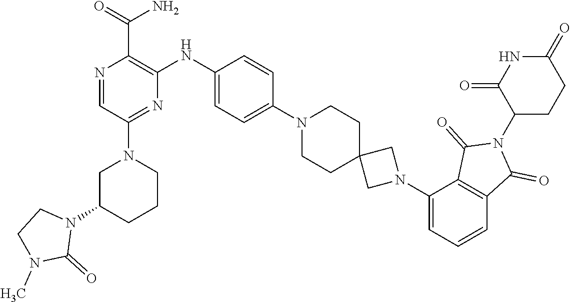 [SMILES: CN1CCN([C@H]2CCCN(c3cnc(C(N)=O)c(Nc4ccc(N5CCC6(CC5)CN(c5cccc7c5C(=O)N(C5CCC(=O)NC5=O)C7=O)C6)cc4)n3)C2)C1=O] |
| 98 | 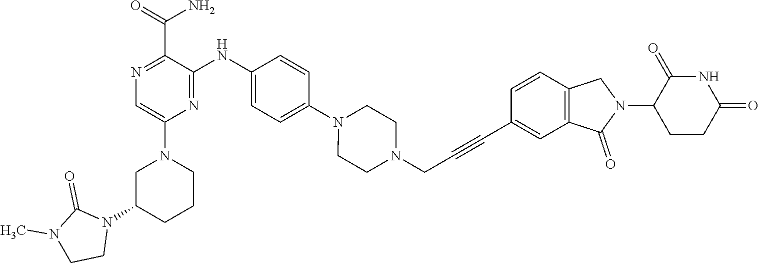 [SMILES: CN1CCN([C@H]2CCCN(c3cnc(C(N)=O)c(Nc4ccc(N5CCN(CC#Cc6ccc7c(c6)C(=O)N(C6CCC(=O)NC6=O)C7)CC5)cc4)n3)C2)C1=O] |
| 99 | 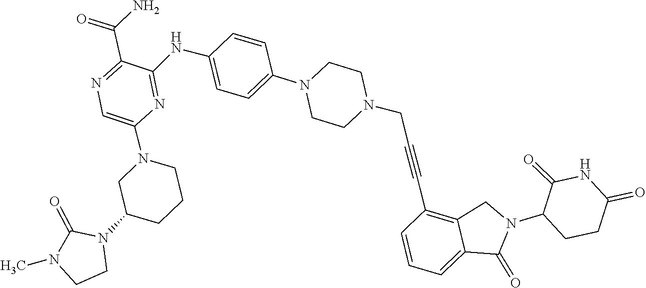 [SMILES: CN1CCN([C@H]2CCCN(c3cnc(C(N)=O)c(Nc4ccc(N5CCN(CC#Cc6cccc7c6CN(C6CCC(=O)NC6=O)C7=O)CC5)cc4)n3)C2)C1=O] |
| 100 | 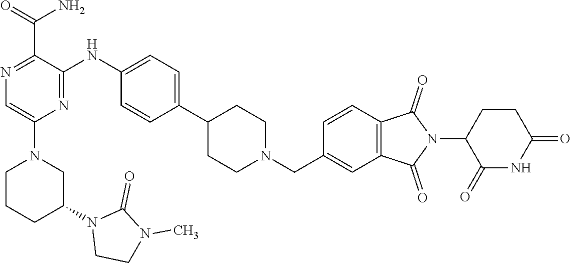 [SMILES: CN1CCN([C@@H]2CCCN(c3cnc(C(N)=O)c(Nc4ccc(C5CCN(Cc6ccc7c(c6)C(=O)N(C6CCC(=O)NC6=O)C7=O)CC5)cc4)n3)C2)C1=O] |
| 101 | 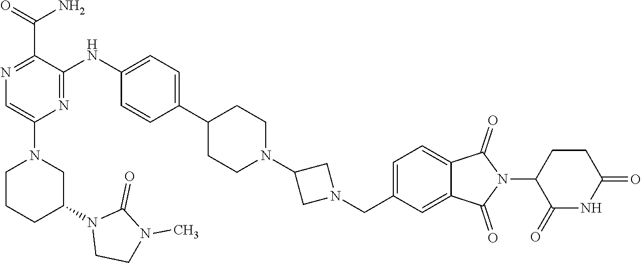 [SMILES: CN1CCN([C@@H]2CCCN(c3cnc(C(N)=O)c(Nc4ccc(C5CCN(C6CN(Cc7ccc8c(c7)C(=O)N(C7CCC(=O)NC7=O)C8=O)C6)CC5)cc4)n3)C2)C1=O] |
| 102 | 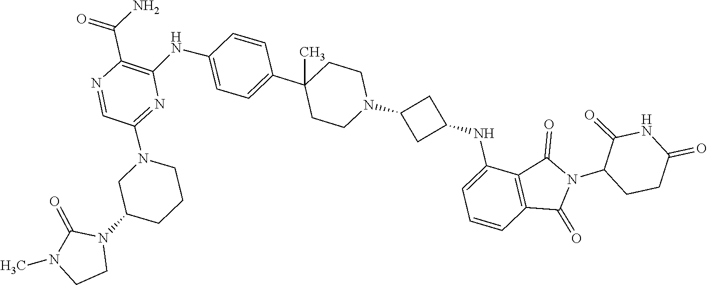 [SMILES: CN1CCN([C@H]2CCCN(c3cnc(C(N)=O)c(Nc4ccc(C5(C)CCN([C@H]6C[C@@H](Nc7cccc8c7C(=O)N(C7CCC(=O)NC7=O)C8=O)C6)CC5)cc4)n3)C2)C1=O] |
| 103 | 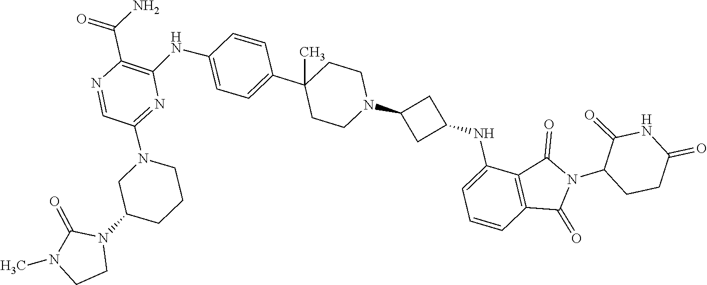 [SMILES: CN1CCN([C@H]2CCCN(c3cnc(C(N)=O)c(Nc4ccc(C5(C)CCN([C@H]6C[C@H](Nc7cccc8c7C(=O)N(C7CCC(=O)NC7=O)C8=O)C6)CC5)cc4)n3)C2)C1=O] |
| 104 | 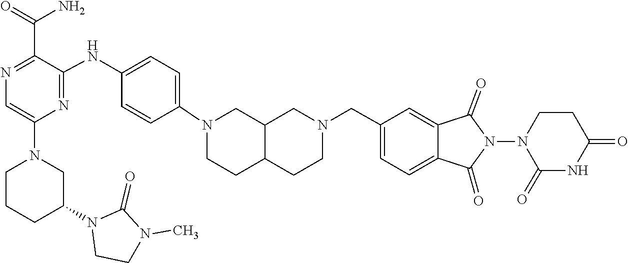 [SMILES: CN1CCN([C@@H]2CCCN(c3cnc(C(N)=O)c(Nc4ccc(N5CCC6CCN(Cc7ccc8c(c7)C(=O)N(N7CCC(=O)NC7=O)C8=O)CC6C5)cc4)n3)C2)C1=O] |
| 105 | 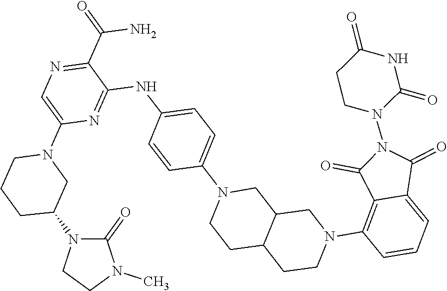 [SMILES: CN1CCN([C@@H]2CCCN(c3cnc(C(N)=O)c(Nc4ccc(N5CCC6CCN(c7cccc8c7C(=O)N(N7CCC(=O)NC7=O)C8=O)CC6C5)cc4)n3)C2)C1=O] |
| 106 | 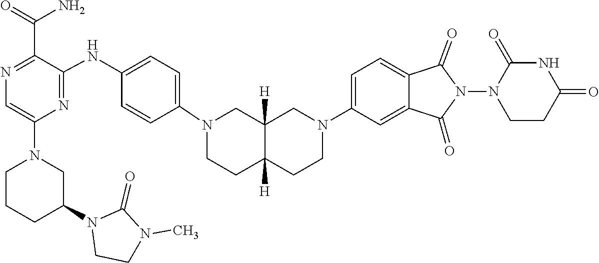 [SMILES: CN1CCN([C@H]2CCCN(c3cnc(C(N)=O)c(Nc4ccc(N5CC[C@H]6CCN(c7ccc8c(c7)C(=O)N(N7CCC(=O)NC7=O)C8=O)C[C@H]6C5)cc4)n3)C2)C1=O] |
| 107 | 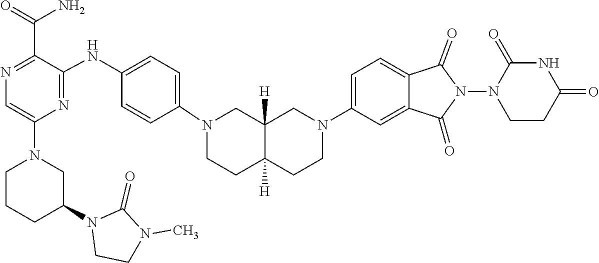 [SMILES: CN1CCN([C@H]2CCCN(c3cnc(C(N)=O)c(Nc4ccc(N5CC[C@@H]6CCN(c7ccc8c(c7)C(=O)N(N7CCC(=O)NC7=O)C8=O)C[C@H]6C5)cc4)n3)C2)C1=O] |
| 108 | 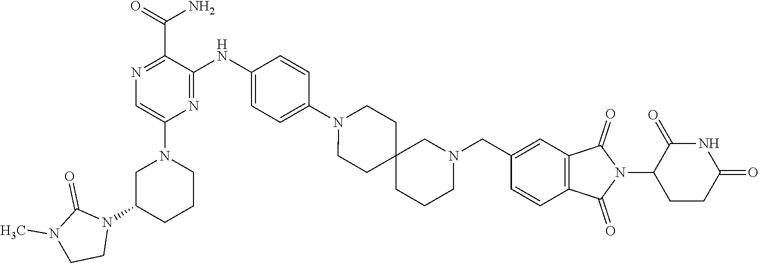 [SMILES: CN1CCN([C@H]2CCCN(c3cnc(C(N)=O)c(Nc4ccc(N5CCC6(CCCN(Cc7ccc8c(c7)C(=O)N(C7CCC(=O)NC7=O)C8=O)C6)CC5)cc4)n3)C2)C1=O] |
| 109 | 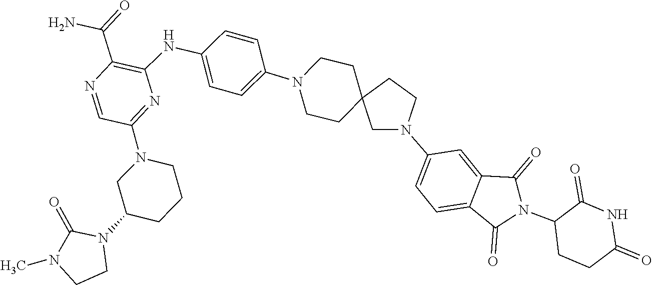 [SMILES: CN1CCN([C@H]2CCCN(c3cnc(C(N)=O)c(Nc4ccc(N5CCC6(CC5)CCN(c5ccc7c(c5)C(=O)N(C5CCC(=O)NC5=O)C7=O)C6)cc4)n3)C2)C1=O] |
| 110 | 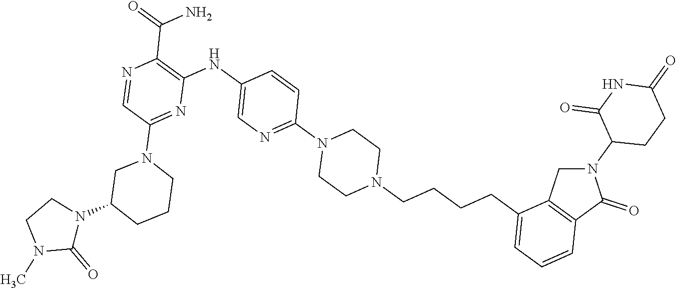 [SMILES: CN1CCN([C@H]2CCCN(c3cnc(C(N)=O)c(Nc4ccc(N5CCN(CCCCc6cccc7c6CN(C6CCC(=O)NC6=O)C7=O)CC5)nc4)n3)C2)C1=O] |
| 111 | 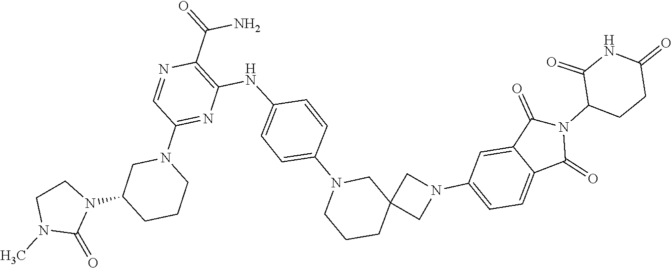 [SMILES: CN1CCN([C@H]2CCCN(c3cnc(C(N)=O)c(Nc4ccc(N5CCCC6(C5)CN(c5ccc7c(c5)C(=O)N(C5CCC(=O)NC5=O)C7=O)C6)cc4)n3)C2)C1=O] |
| 112 | 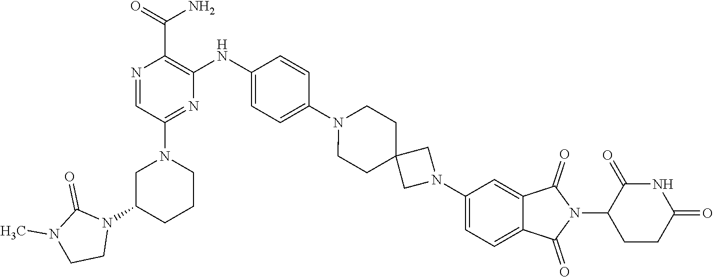 [SMILES: CN1CCN([C@H]2CCCN(c3cnc(C(N)=O)c(Nc4ccc(N5CCC6(CC5)CN(c5ccc7c(c5)C(=O)N(C5CCC(=O)NC5=O)C7=O)C6)cc4)n3)C2)C1=O] |
| 113 | 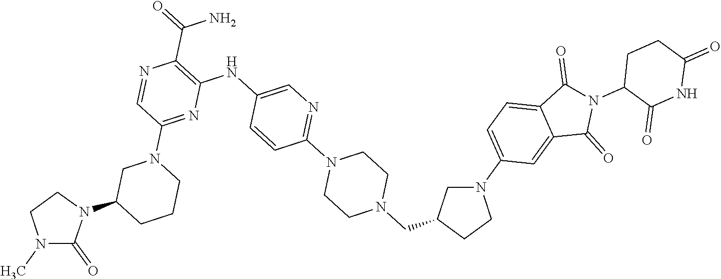 [SMILES: CN1CCN([C@@H]2CCCN(c3cnc(C(N)=O)c(Nc4ccc(N5CCN(C[C@H]6CCN(c7ccc8c(c7)C(=O)N(C7CCC(=O)NC7=O)C8=O)C6)CC5)nc4)n3)C2)C1=O] |
| 114 | 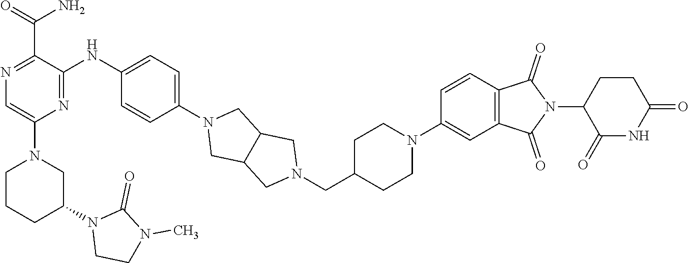 [SMILES: CN1CCN([C@@H]2CCCN(c3cnc(C(N)=O)c(Nc4ccc(N5CC6CN(CC7CCN(c8ccc9c(c8)C(=O)N(C8CCC(=O)NC8=O)C9=O)CC7)CC6C5)cc4)n3)C2)C1=O] |
| 115 | 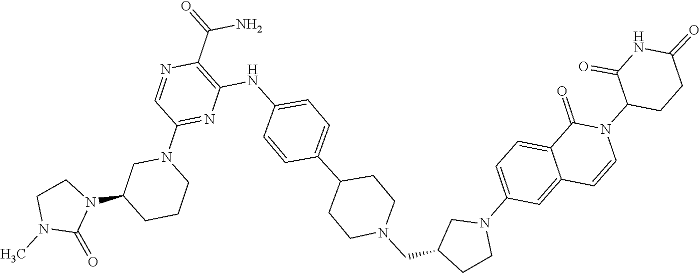 [SMILES: CN1CCN([C@@H]2CCCN(c3cnc(C(N)=O)c(Nc4ccc(C5CCN(C[C@H]6CCN(c7ccc8c(=O)n(C9CCC(=O)NC9=O)ccc8c7)C6)CC5)cc4)n3)C2)C1=O] |
| 116 | 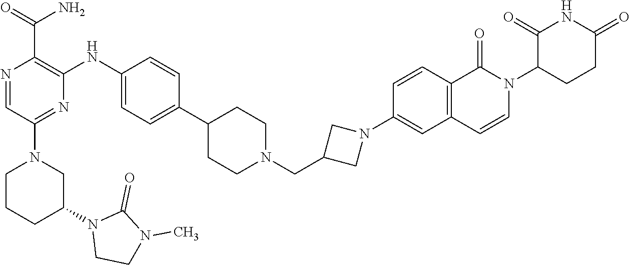 [SMILES: CN1CCN([C@@H]2CCCN(c3cnc(C(N)=O)c(Nc4ccc(C5CCN(CC6CN(c7ccc8c(=O)n(C9CCC(=O)NC9=O)ccc8c7)C6)CC5)cc4)n3)C2)C1=O] |
| 117 | 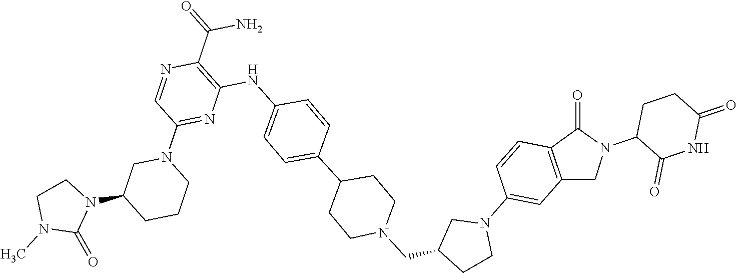 [SMILES: CN1CCN([C@@H]2CCCN(c3cnc(C(N)=O)c(Nc4ccc(C5CCN(C[C@H]6CCN(c7ccc8c(c7)CN(C7CCC(=O)NC7=O)C8=O)C6)CC5)cc4)n3)C2)C1=O] |
| 118 | 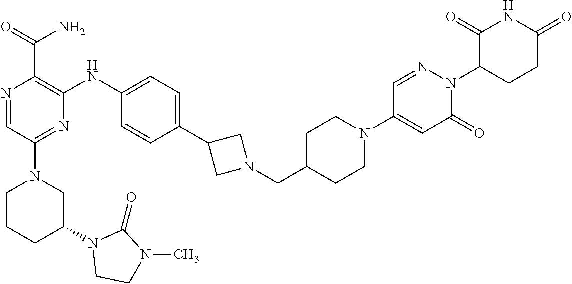 [SMILES: CN1CCN([C@@H]2CCCN(c3cnc(C(N)=O)c(Nc4ccc(C5CN(CC6CCN(c7cnn(C8CCC(=O)NC8=O)c(=O)c7)CC6)C5)cc4)n3)C2)C1=O] |
| 119 | 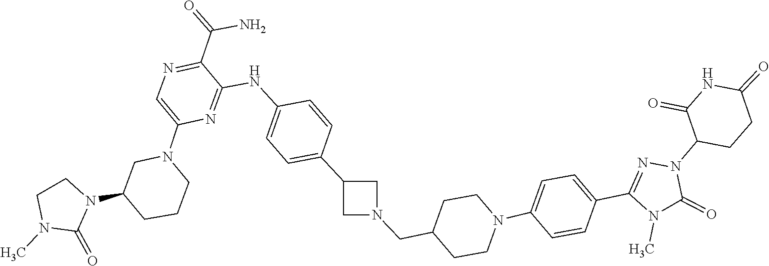 [SMILES: CN1CCN([C@@H]2CCCN(c3cnc(C(N)=O)c(Nc4ccc(C5CN(CC6CCN(c7ccc(-c8nn(C9CCC(=O)NC9=O)c(=O)n8C)cc7)CC6)C5)cc4)n3)C2)C1=O] |
| 120 | 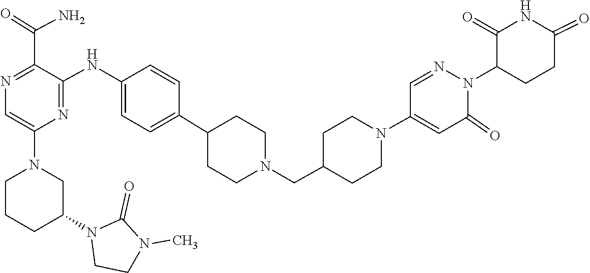 [SMILES: CN1CCN([C@@H]2CCCN(c3cnc(C(N)=O)c(Nc4ccc(C5CCN(CC6CCN(c7cnn(C8CCC(=O)NC8=O)c(=O)c7)CC6)CC5)cc4)n3)C2)C1=O] |
| 121 | 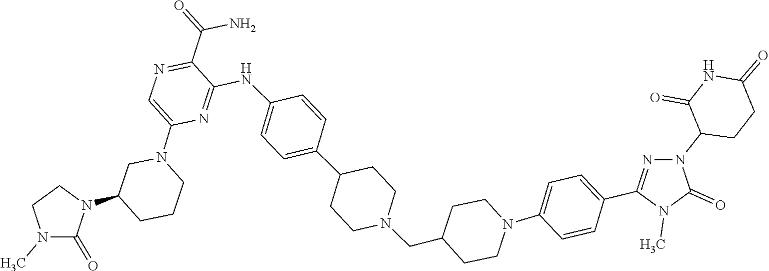 [SMILES: CN1CCN([C@@H]2CCCN(c3cnc(C(N)=O)c(Nc4ccc(C5CCN(CC6CCN(c7ccc(-c8nn(C9CCC(=O)NC9=O)c(=O)n8C)cc7)CC6)CC5)cc4)n3)C2)C1=O] |
| 122 | 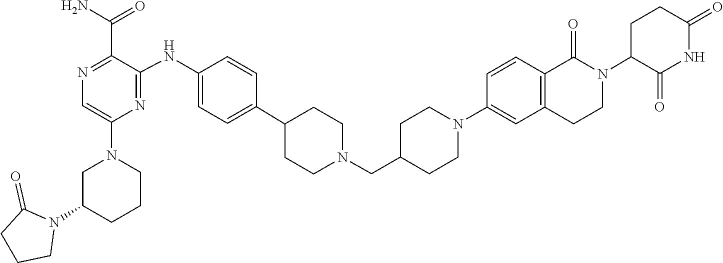 [SMILES: NC(=O)c1ncc(N2CCC[C@H](N3CCCC3=O)C2)nc1Nc1ccc(C2CCN(CC3CCN(c4ccc5c(c4)CCN(C4CCC(=O)NC4=O)C5=O)CC3)CC2)cc1] |
| 123 | 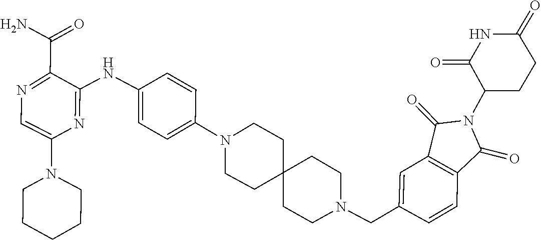 [SMILES: NC(=O)c1ncc(N2CCCCC2)nc1Nc1ccc(N2CCC3(CCN(Cc4ccc5c(c4)C(=O)N(C4CCC(=O)NC4=O)C5=O)CC3)CC2)cc1] |
| 124 | 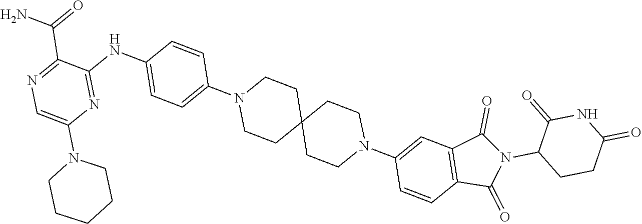 [SMILES: NC(=O)c1ncc(N2CCCCC2)nc1Nc1ccc(N2CCC3(CC2)CCN(c2ccc4c(c2)C(=O)N(C2CCC(=O)NC2=O)C4=O)CC3)cc1] |
| 125 | 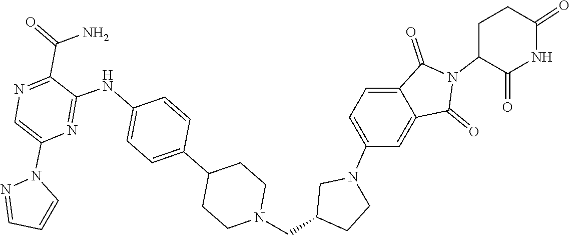 [SMILES: NC(=O)c1ncc(-n2cccn2)nc1Nc1ccc(C2CCN(C[C@H]3CCN(c4ccc5c(c4)C(=O)N(C4CCC(=O)NC4=O)C5=O)C3)CC2)cc1] |
| 126 | 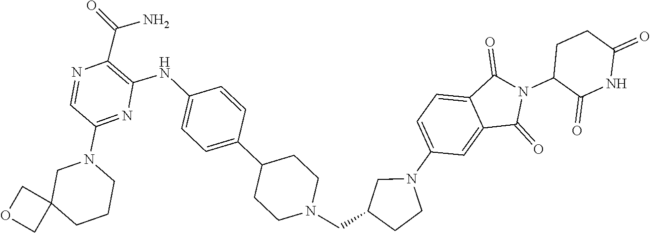 [SMILES: NC(=O)c1ncc(N2CCCC3(COC3)C2)nc1Nc1ccc(C2CCN(C[C@H]3CCN(c4ccc5c(c4)C(=O)N(C4CCC(=O)NC4=O)C5=O)C3)CC2)cc1] |
| 127 | 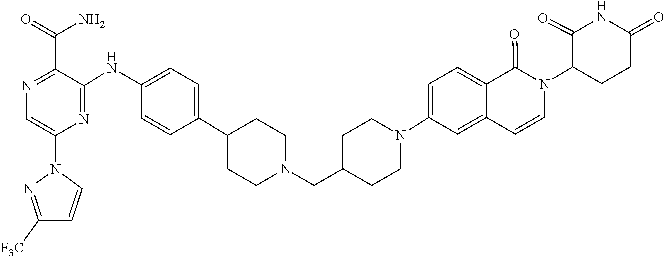 [SMILES: NC(=O)c1ncc(-n2ccc(C(F)(F)F)n2)nc1Nc1ccc(C2CCN(CC3CCN(c4ccc5c(=O)n(C6CCC(=O)NC6=O)ccc5c4)CC3)CC2)cc1] |
| 128 | 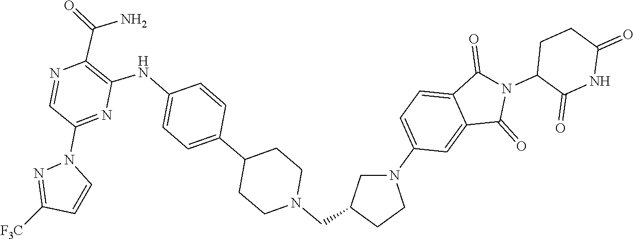 [SMILES: NC(=O)c1ncc(-n2ccc(C(F)(F)F)n2)nc1Nc1ccc(C2CCN(C[C@H]3CCN(c4ccc5c(c4)C(=O)N(C4CCC(=O)NC4=O)C5=O)C3)CC2)cc1] |
| 129 | 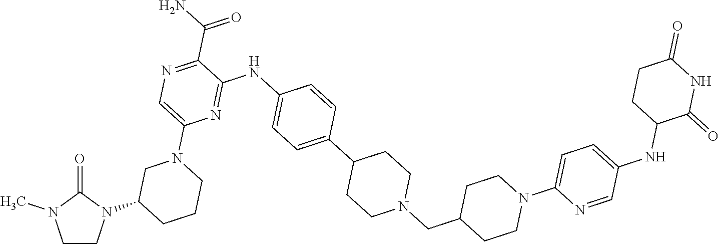 [SMILES: CN1CCN([C@H]2CCCN(c3cnc(C(N)=O)c(Nc4ccc(C5CCN(CC6CCN(c7ccc(NC8CCC(=O)NC8=O)cn7)CC6)CC5)cc4)n3)C2)C1=O] |
| 130 | 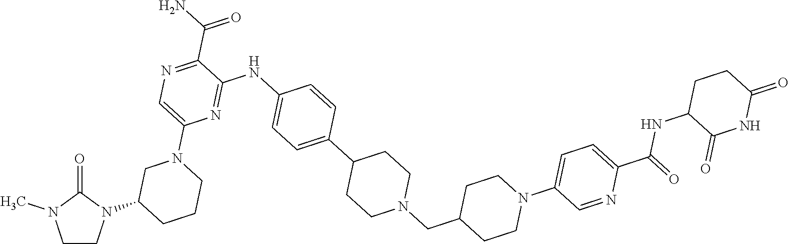 [SMILES: CN1CCN([C@H]2CCCN(c3cnc(C(N)=O)c(Nc4ccc(C5CCN(CC6CCN(c7ccc(C(=O)NC8CCC(=O)NC8=O)nc7)CC6)CC5)cc4)n3)C2)C1=O] |
| 131 | 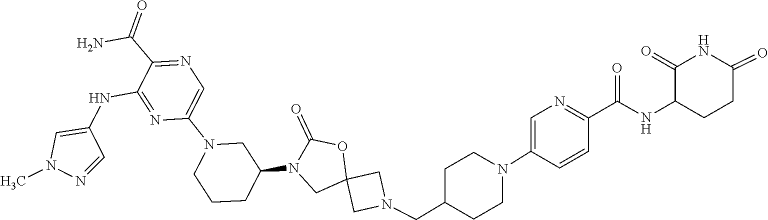 [SMILES: Cn1cc(Nc2nc(N3CCC[C@H](N4CC5(CN(CC6CCN(c7ccc(C(=O)NC8CCC(=O)NC8=O)nc7)CC6)C5)OC4=O)C3)cnc2C(N)=O)cn1] |
| 132 | 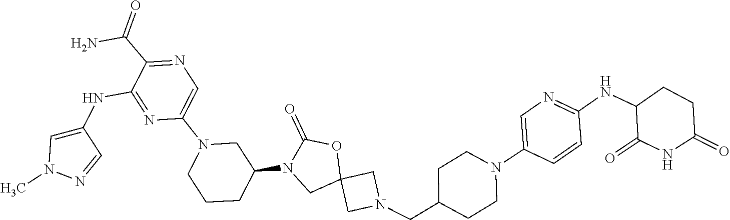 [SMILES: Cn1cc(Nc2nc(N3CCC[C@H](N4CC5(CN(CC6CCN(c7ccc(NC8CCC(=O)NC8=O)nc7)CC6)C5)OC4=O)C3)cnc2C(N)=O)cn1] |
| 133 | 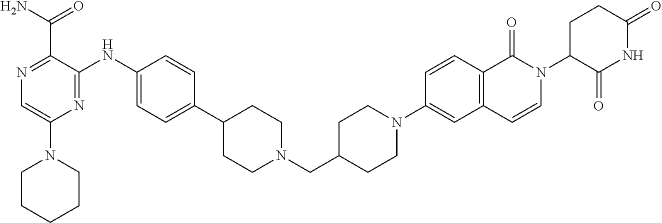 [SMILES: NC(=O)c1ncc(N2CCCCC2)nc1Nc1ccc(C2CCN(CC3CCN(c4ccc5c(=O)n(C6CCC(=O)NC6=O)ccc5c4)CC3)CC2)cc1] |
| 134 | 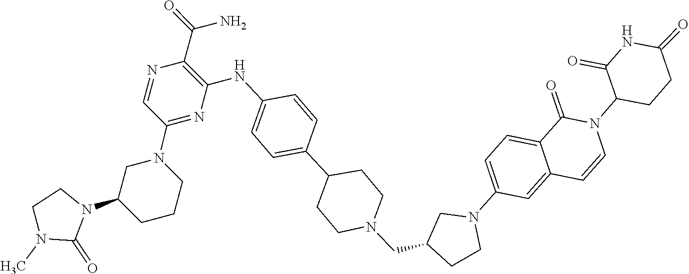 [SMILES: CN1CCN([C@@H]2CCCN(c3cnc(C(N)=O)c(Nc4ccc(C5CCN(C[C@H]6CCN(c7ccc8c(=O)n(C9CCC(=O)NC9=O)ccc8c7)C6)CC5)cc4)n3)C2)C1=O] |
| 135 | 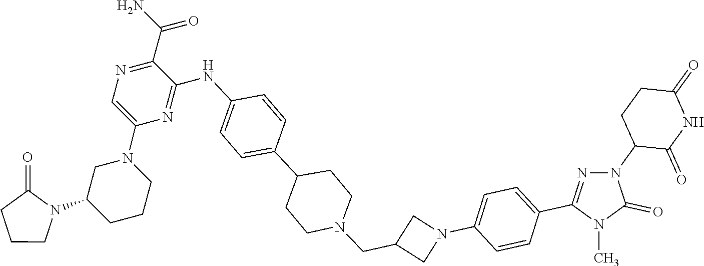 [SMILES: Cn1c(-c2ccc(N3CC(CN4CCC(c5ccc(Nc6nc(N7CCC[C@H](N8CCCC8=O)C7)cnc6C(N)=O)cc5)CC4)C3)cc2)nn(C2CCC(=O)NC2=O)c1=O] |
| 136 | 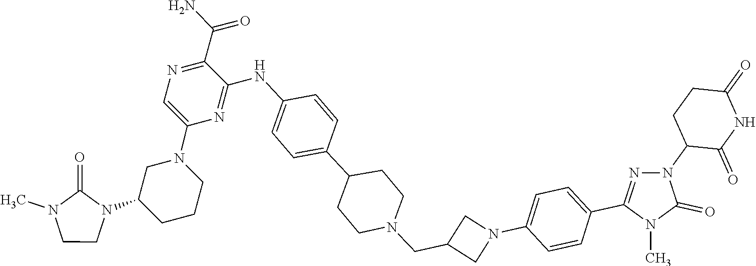 [SMILES: CN1CCN([C@H]2CCCN(c3cnc(C(N)=O)c(Nc4ccc(C5CCN(CC6CN(c7ccc(-c8nn(C9CCC(=O)NC9=O)c(=O)n8C)cc7)C6)CC5)cc4)n3)C2)C1=O] |
| 137 | 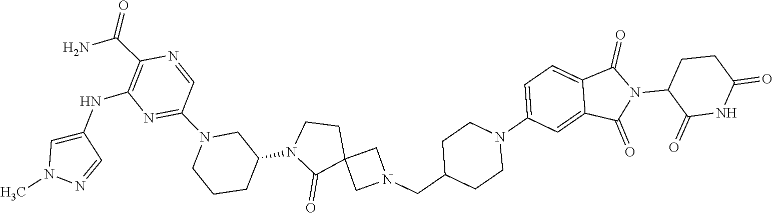 [SMILES: Cn1cc(Nc2nc(N3CCC[C@@H](N4CCC5(CN(CC6CCN(c7ccc8c(c7)C(=O)N(C7CCC(=O)NC7=O)C8=O)CC6)C5)C4=O)C3)cnc2C(N)=O)cn1] |
| 138 | 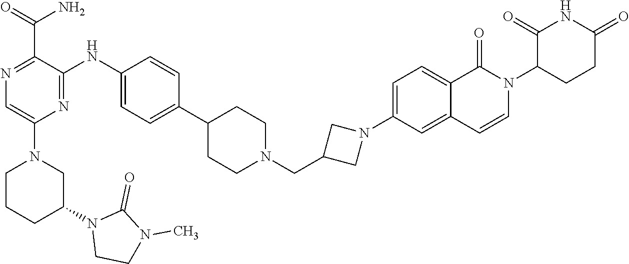 [SMILES: CN1CCN([C@@H]2CCCN(c3cnc(C(N)=O)c(Nc4ccc(C5CCN(CC6CN(c7ccc8c(=O)n(C9CCC(=O)NC9=O)ccc8c7)C6)CC5)cc4)n3)C2)C1=O] |
| 139 | 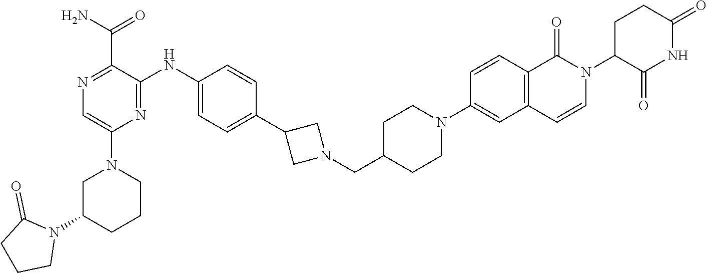 [SMILES: NC(=O)c1ncc(N2CCC[C@H](N3CCCC3=O)C2)nc1Nc1ccc(C2CN(CC3CCN(c4ccc5c(=O)n(C6CCC(=O)NC6=O)ccc5c4)CC3)C2)cc1] |
| 140 | 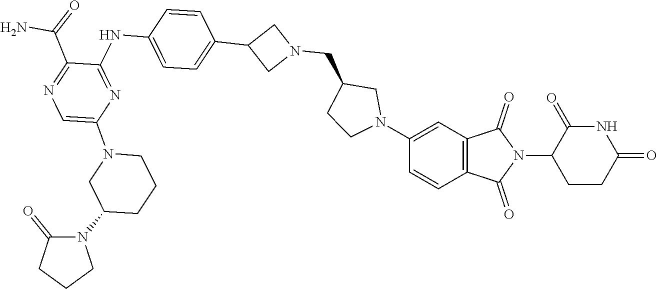 [SMILES: NC(=O)c1ncc(N2CCC[C@H](N3CCCC3=O)C2)nc1Nc1ccc(C2CN(C[C@@H]3CCN(c4ccc5c(c4)C(=O)N(C4CCC(=O)NC4=O)C5=O)C3)C2)cc1] |
| 141 | 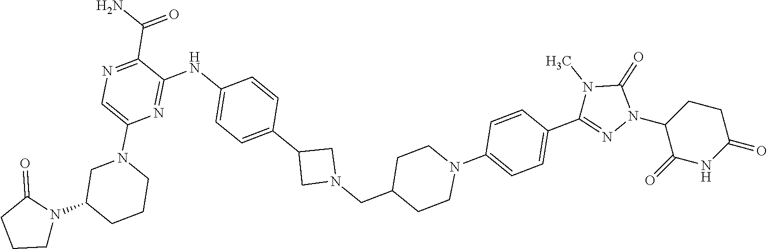 [SMILES: Cn1c(-c2ccc(N3CCC(CN4CC(c5ccc(Nc6nc(N7CCC[C@H](N8CCCC8=O)C7)cnc6C(N)=O)cc5)C4)CC3)cc2)nn(C2CCC(=O)NC2=O)c1=O] |
| 142 | 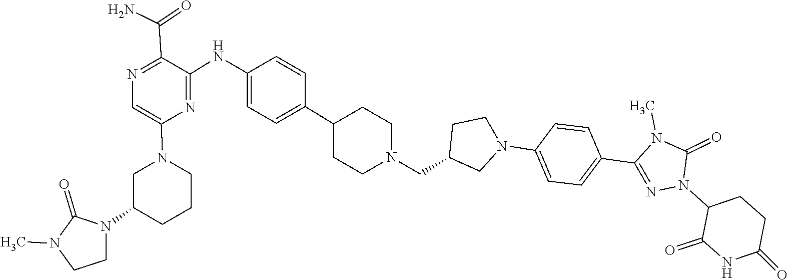 [SMILES: CN1CCN([C@H]2CCCN(c3cnc(C(N)=O)c(Nc4ccc(C5CCN(C[C@@H]6CCN(c7ccc(-c8nn(C9CCC(=O)NC9=O)c(=O)n8C)cc7)C6)CC5)cc4)n3)C2)C1=O] |
| 143 | 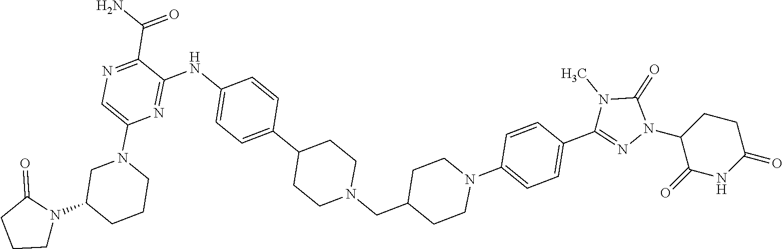 [SMILES: Cn1c(-c2ccc(N3CCC(CN4CCC(c5ccc(Nc6nc(N7CCC[C@H](N8CCCC8=O)C7)cnc6C(N)=O)cc5)CC4)CC3)cc2)nn(C2CCC(=O)NC2=O)c1=O] |
| 144 | 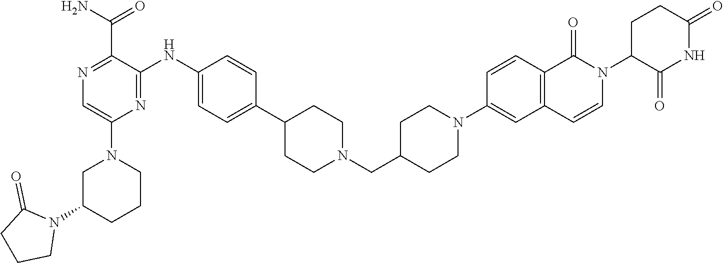 [SMILES: NC(=O)c1ncc(N2CCC[C@H](N3CCCC3=O)C2)nc1Nc1ccc(C2CCN(CC3CCN(c4ccc5c(=O)n(C6CCC(=O)NC6=O)ccc5c4)CC3)CC2)cc1] |
| 145 | 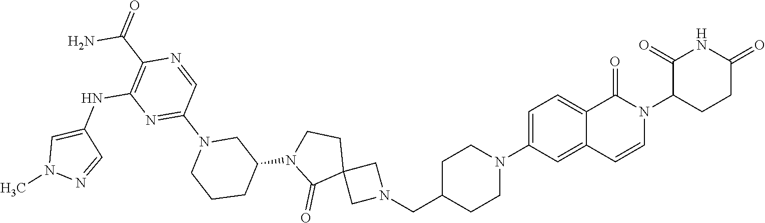 [SMILES: Cn1cc(Nc2nc(N3CCC[C@@H](N4CCC5(CN(CC6CCN(c7ccc8c(=O)n(C9CCC(=O)NC9=O)ccc8c7)CC6)C5)C4=O)C3)cnc2C(N)=O)cn1] |
| 146 | 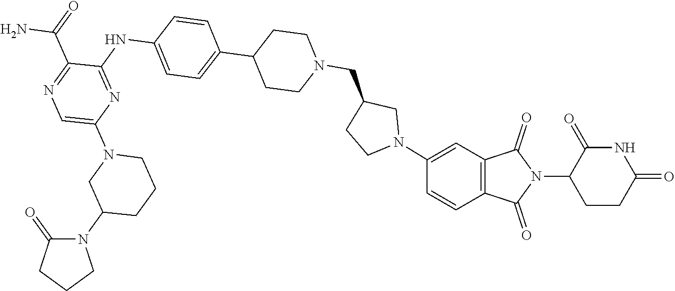 [SMILES: NC(=O)c1ncc(N2CCCC(N3CCCC3=O)C2)nc1Nc1ccc(C2CCN(C[C@@H]3CCN(c4ccc5c(c4)C(=O)N(C4CCC(=O)NC4=O)C5=O)C3)CC2)cc1] |
| 147 | 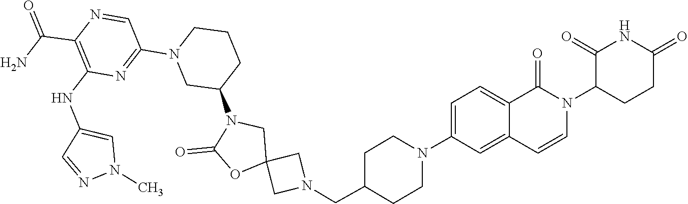 [SMILES: Cn1cc(Nc2nc(N3CCC[C@@H](N4CC5(CN(CC6CCN(c7ccc8c(=O)n(C9CCC(=O)NC9=O)ccc8c7)CC6)C5)OC4=O)C3)cnc2C(N)=O)cn1] |
| 148 | 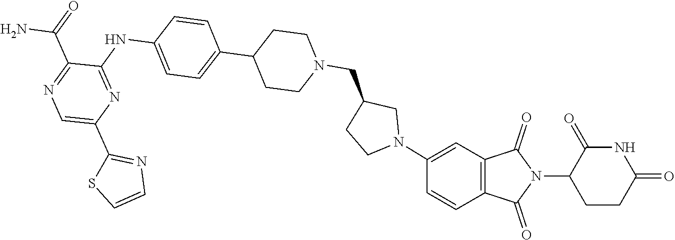 [SMILES: NC(=O)c1ncc(-c2nccs2)nc1Nc1ccc(C2CCN(C[C@@H]3CCN(c4ccc5c(c4)C(=O)N(C4CCC(=O)NC4=O)C5=O)C3)CC2)cc1] |
| 149 | 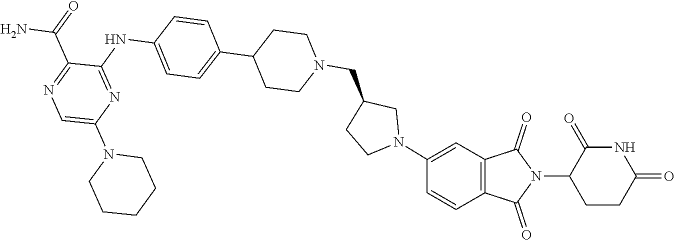 [SMILES: NC(=O)c1ncc(N2CCCCC2)nc1Nc1ccc(C2CCN(C[C@@H]3CCN(c4ccc5c(c4)C(=O)N(C4CCC(=O)NC4=O)C5=O)C3)CC2)cc1] |
| 150 | 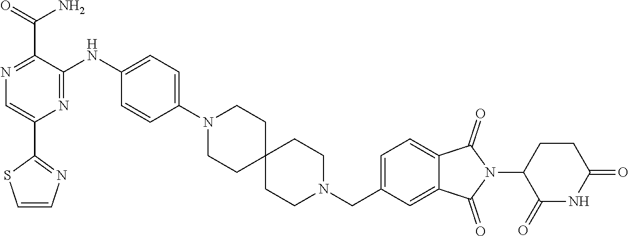 [SMILES: NC(=O)c1ncc(-c2nccs2)nc1Nc1ccc(N2CCC3(CCN(Cc4ccc5c(c4)C(=O)N(C4CCC(=O)NC4=O)C5=O)CC3)CC2)cc1] |
| 151 | 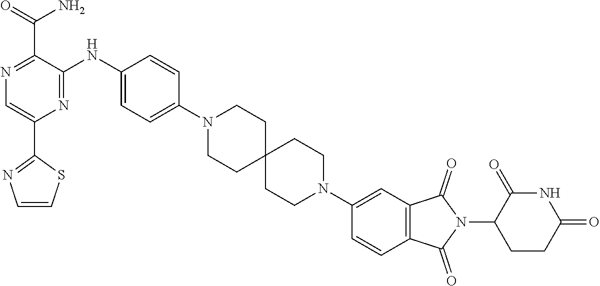 [SMILES: NC(=O)c1ncc(-c2nccs2)nc1Nc1ccc(N2CCC3(CC2)CCN(c2ccc4c(c2)C(=O)N(C2CCC(=O)NC2=O)C4=O)CC3)cc1] |
| 152 | 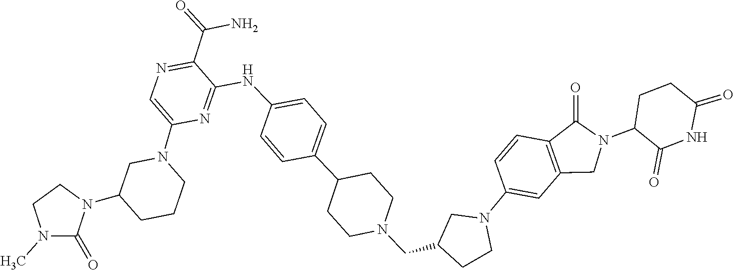 |
| 153 | 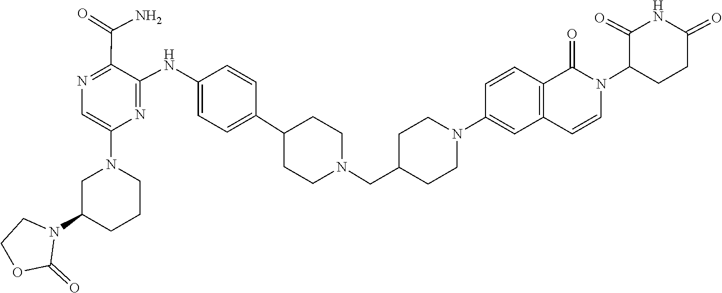 [SMILES: NC(=O)c1ncc(N2CCC[C@@H](N3CCOC3=O)C2)nc1Nc1ccc(C2CCN(CC3CCN(c4ccc5c(=O)n(C6CCC(=O)NC6=O)ccc5c4)CC3)CC2)cc1] |
| 154 | 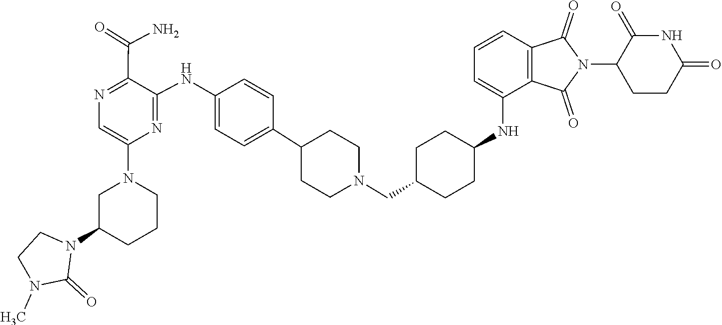 [SMILES: CN1CCN([C@@H]2CCCN(c3cnc(C(N)=O)c(Nc4ccc(C5CCN(C[C@H]6CC[C@H](Nc7cccc8c7C(=O)N(C7CCC(=O)NC7=O)C8=O)CC6)CC5)cc4)n3)C2)C1=O] |
| 155 | 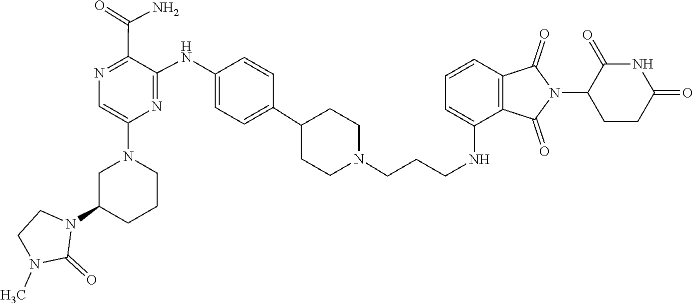 [SMILES: CN1CCN([C@@H]2CCCN(c3cnc(C(N)=O)c(Nc4ccc(C5CCN(CCCNc6cccc7c6C(=O)N(C6CCC(=O)NC6=O)C7=O)CC5)cc4)n3)C2)C1=O] |
| 156 | 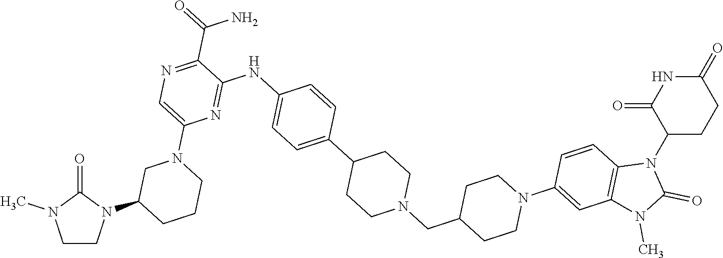 [SMILES: CN1CCN([C@@H]2CCCN(c3cnc(C(N)=O)c(Nc4ccc(C5CCN(CC6CCN(c7ccc8c(c7)n(C)c(=O)n8C7CCC(=O)NC7=O)CC6)CC5)cc4)n3)C2)C1=O] |
| 157 | 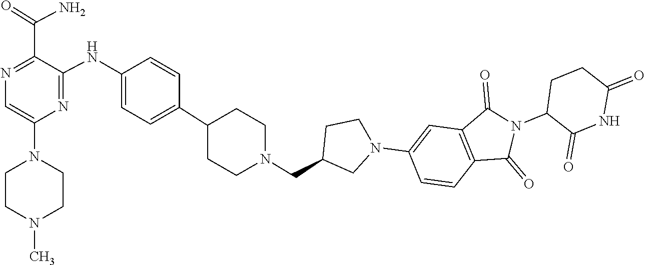 [SMILES: CN1CCN(c2cnc(C(N)=O)c(Nc3ccc(C4CCN(C[C@H]5CCN(c6ccc7c(c6)C(=O)N(C6CCC(=O)NC6=O)C7=O)C5)CC4)cc3)n2)CC1] |
| 158 | 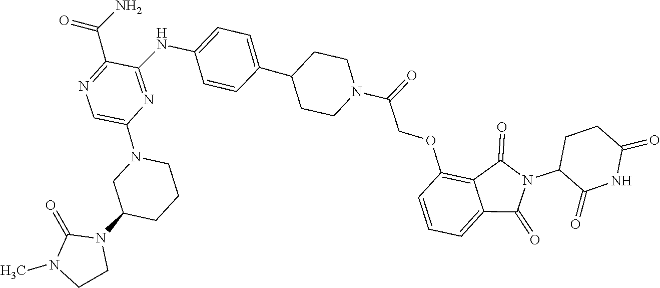 [SMILES: CN1CCN([C@@H]2CCCN(c3cnc(C(N)=O)c(Nc4ccc(C5CCN(C(=O)COc6cccc7c6C(=O)N(C6CCC(=O)NC6=O)C7=O)CC5)cc4)n3)C2)C1=O] |
| 159 | 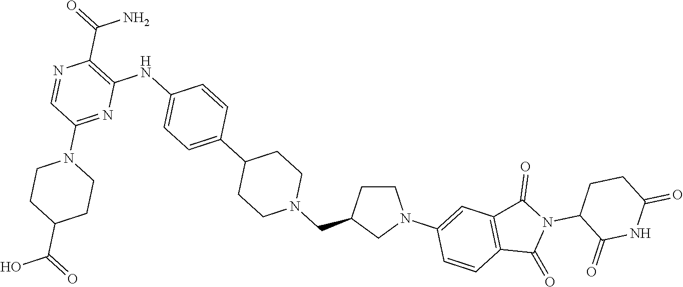 [SMILES: NC(=O)c1ncc(N2CCC(C(=O)O)CC2)nc1Nc1ccc(C2CCN(C[C@H]3CCN(c4ccc5c(c4)C(=O)N(C4CCC(=O)NC4=O)C5=O)C3)CC2)cc1] |
| 160 | 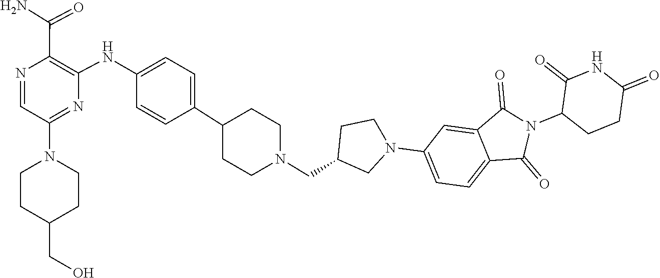 [SMILES: NC(=O)c1ncc(N2CCC(CO)CC2)nc1Nc1ccc(C2CCN(C[C@@H]3CCN(c4ccc5c(c4)C(=O)N(C4CCC(=O)NC4=O)C5=O)C3)CC2)cc1] |
| 161 | 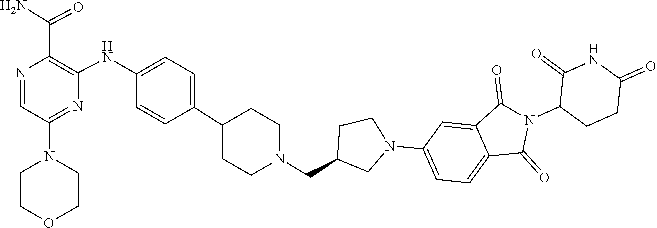 [SMILES: NC(=O)c1ncc(N2CCOCC2)nc1Nc1ccc(C2CCN(C[C@H]3CCN(c4ccc5c(c4)C(=O)N(C4CCC(=O)NC4=O)C5=O)C3)CC2)cc1] |
| 162 | 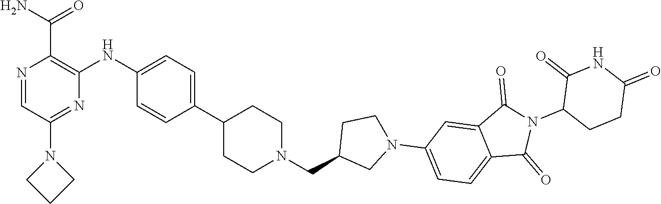 [SMILES: NC(=O)c1ncc(N2CCC2)nc1Nc1ccc(C2CCN(C[C@H]3CCN(c4ccc5c(c4)C(=O)N(C4CCC(=O)NC4=O)C5=O)C3)CC2)cc1] |
| 163 | 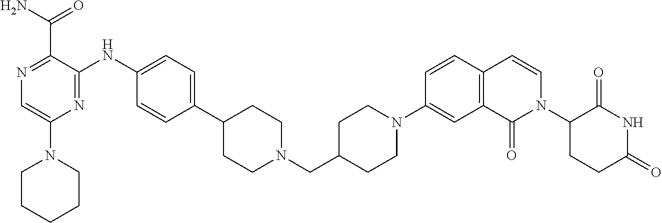 [SMILES: NC(=O)c1ncc(N2CCCCC2)nc1Nc1ccc(C2CCN(CC3CCN(c4ccc5ccn(C6CCC(=O)NC6=O)c(=O)c5c4)CC3)CC2)cc1] |
| 164 | 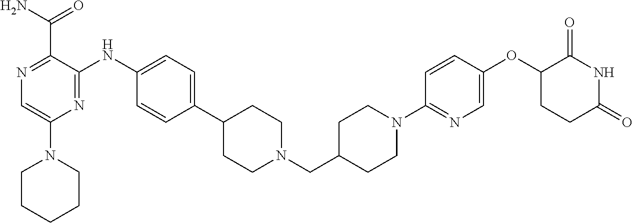 [SMILES: NC(=O)c1ncc(N2CCCCC2)nc1Nc1ccc(C2CCN(CC3CCN(c4ccc(OC5CCC(=O)NC5=O)cn4)CC3)CC2)cc1] |
| 165 | 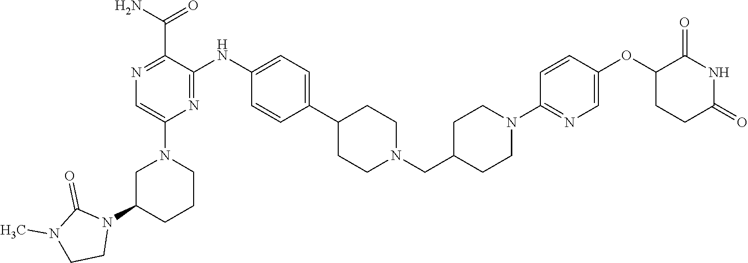 [SMILES: CN1CCN([C@@H]2CCCN(c3cnc(C(N)=O)c(Nc4ccc(C5CCN(CC6CCN(c7ccc(OC8CCC(=O)NC8=O)cn7)CC6)CC5)cc4)n3)C2)C1=O] |
| 166 | 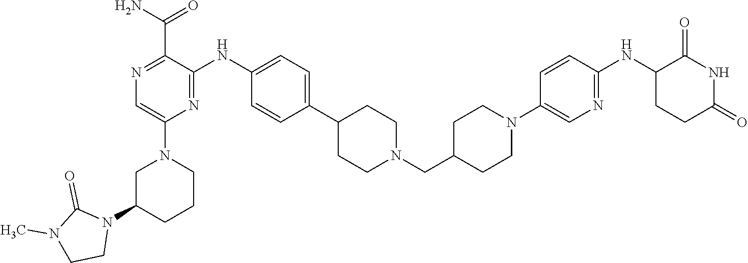 [SMILES: CN1CCN([C@@H]2CCCN(c3cnc(C(N)=O)c(Nc4ccc(C5CCN(CC6CCN(c7ccc(NC8CCC(=O)NC8=O)nc7)CC6)CC5)cc4)n3)C2)C1=O] |
| 167 | 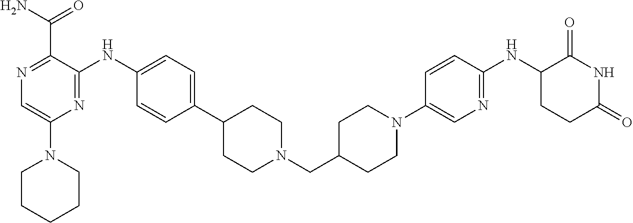 [SMILES: NC(=O)c1ncc(N2CCCCC2)nc1Nc1ccc(C2CCN(CC3CCN(c4ccc(NC5CCC(=O)NC5=O)nc4)CC3)CC2)cc1] |
| 168 | 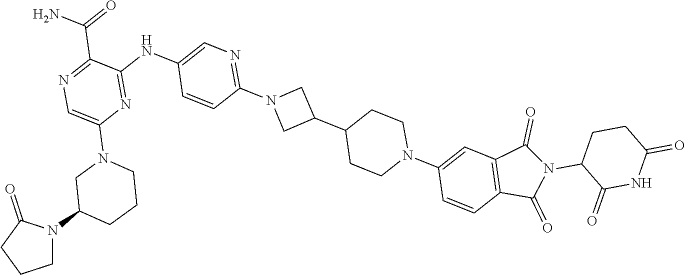 [SMILES: NC(=O)c1ncc(N2CCC[C@@H](N3CCCC3=O)C2)nc1Nc1ccc(N2CC(C3CCN(c4ccc5c(c4)C(=O)N(C4CCC(=O)NC4=O)C5=O)CC3)C2)nc1] |
| 169 | 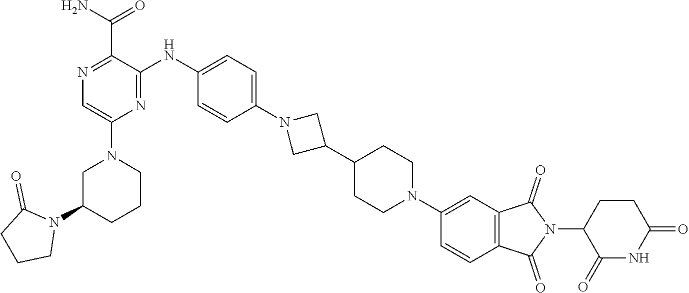 [SMILES: NC(=O)c1ncc(N2CCC[C@@H](N3CCCC3=O)C2)nc1Nc1ccc(N2CC(C3CCN(c4ccc5c(c4)C(=O)N(C4CCC(=O)NC4=O)C5=O)CC3)C2)cc1] |
| 170 | 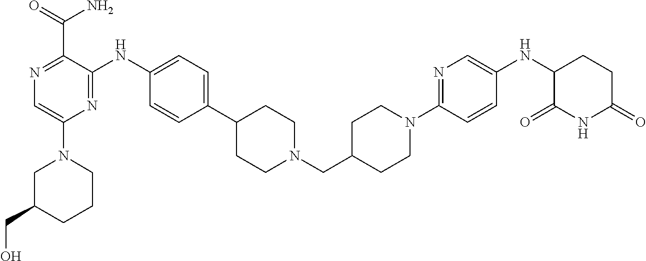 [SMILES: NC(=O)c1ncc(N2CCC[C@@H](CO)C2)nc1Nc1ccc(C2CCN(CC3CCN(c4ccc(NC5CCC(=O)NC5=O)cn4)CC3)CC2)cc1] |
| 171 | 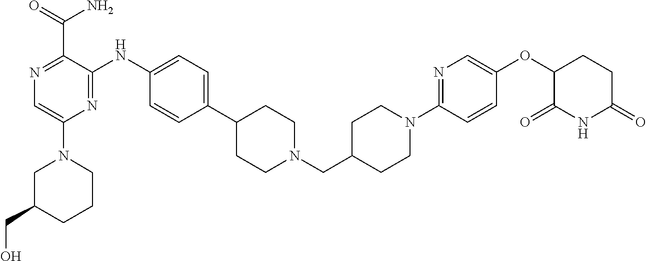 [SMILES: NC(=O)c1ncc(N2CCC[C@@H](CO)C2)nc1Nc1ccc(C2CCN(CC3CCN(c4ccc(OC5CCC(=O)NC5=O)cn4)CC3)CC2)cc1] |
| 172 | 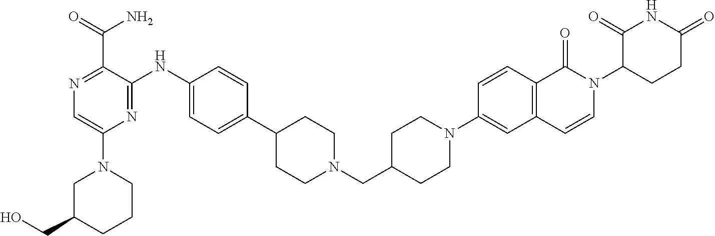 [SMILES: NC(=O)c1ncc(N2CCC[C@@H](CO)C2)nc1Nc1ccc(C2CCN(CC3CCN(c4ccc5c(=O)n(C6CCC(=O)NC6=O)ccc5c4)CC3)CC2)cc1] |
| 173 | 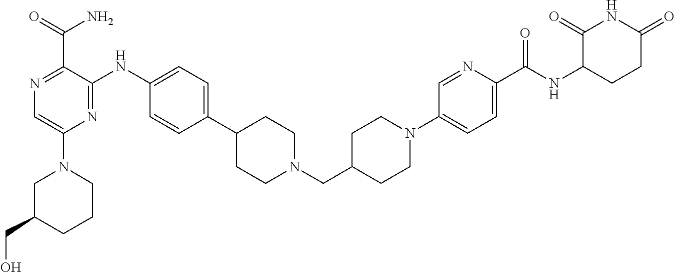 [SMILES: NC(=O)c1ncc(N2CCC[C@@H](CO)C2)nc1Nc1ccc(C2CCN(CC3CCN(c4ccc(C(=O)NC5CCC(=O)NC5=O)nc4)CC3)CC2)cc1] |
| 174 | 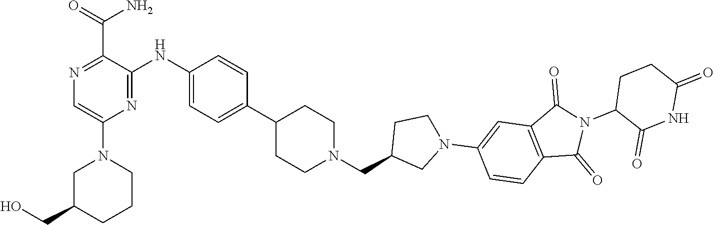 [SMILES: NC(=O)c1ncc(N2CCC[C@@H](CO)C2)nc1Nc1ccc(C2CCN(C[C@H]3CCN(c4ccc5c(c4)C(=O)N(C4CCC(=O)NC4=O)C5=O)C3)CC2)cc1] |
| 175 | 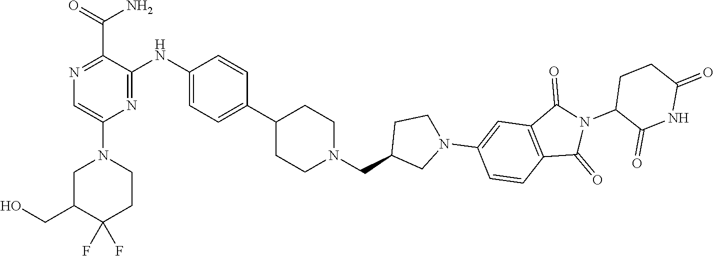 [SMILES: NC(=O)c1ncc(N2CCC(F)(F)C(CO)C2)nc1Nc1ccc(C2CCN(C[C@H]3CCN(c4ccc5c(c4)C(=O)N(C4CCC(=O)NC4=O)C5=O)C3)CC2)cc1] |
| 176 | 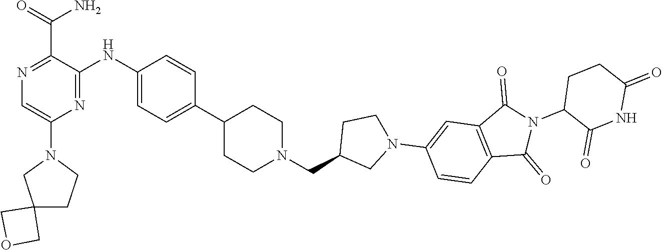 [SMILES: NC(=O)c1ncc(N2CCC3(COC3)C2)nc1Nc1ccc(C2CCN(C[C@H]3CCN(c4ccc5c(c4)C(=O)N(C4CCC(=O)NC4=O)C5=O)C3)CC2)cc1] |
| 177 | 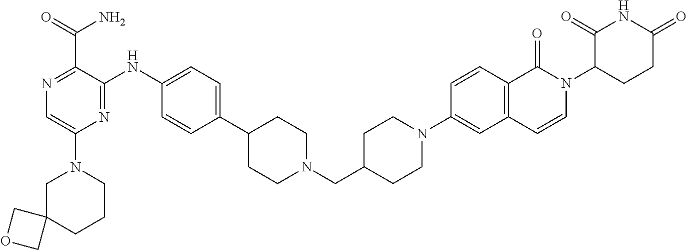 [SMILES: NC(=O)c1ncc(N2CCCC3(COC3)C2)nc1Nc1ccc(C2CCN(CC3CCN(c4ccc5c(=O)n(C6CCC(=O)NC6=O)ccc5c4)CC3)CC2)cc1] |
| 178 | 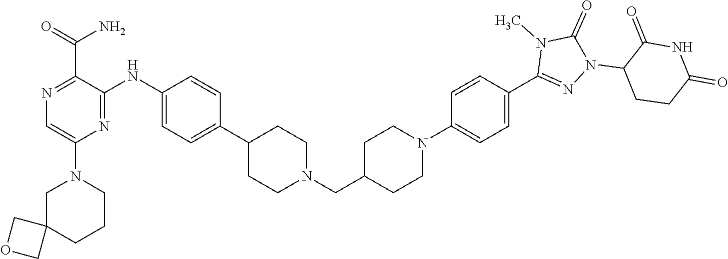 [SMILES: Cn1c(-c2ccc(N3CCC(CN4CCC(c5ccc(Nc6nc(N7CCCC8(COC8)C7)cnc6C(N)=O)cc5)CC4)CC3)cc2)nn(C2CCC(=O)NC2=O)c1=O] |
| 179 | 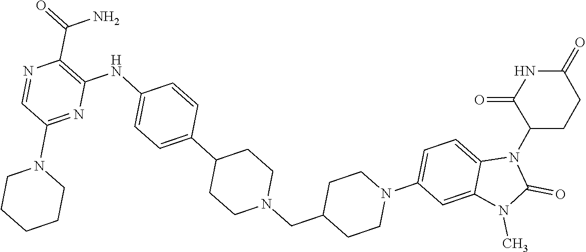 [SMILES: Cn1c(=O)n(C2CCC(=O)NC2=O)c2ccc(N3CCC(CN4CCC(c5ccc(Nc6nc(N7CCCCC7)cnc6C(N)=O)cc5)CC4)CC3)cc21] |
| 180 | 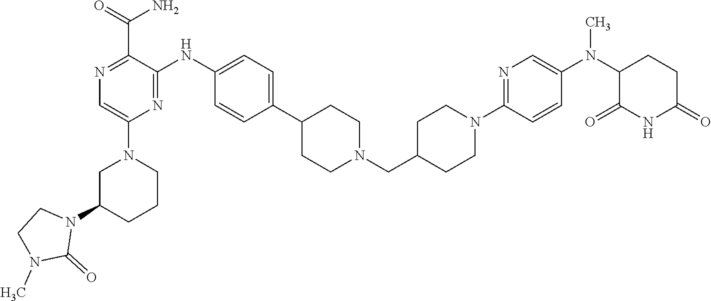 [SMILES: CN1CCN([C@@H]2CCCN(c3cnc(C(N)=O)c(Nc4ccc(C5CCN(CC6CCN(c7ccc(N(C)C8CCC(=O)NC8=O)cn7)CC6)CC5)cc4)n3)C2)C1=O] |
| 181 | 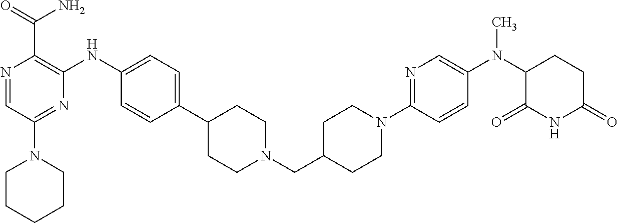 [SMILES: CN(c1ccc(N2CCC(CN3CCC(c4ccc(Nc5nc(N6CCCCC6)cnc5C(N)=O)cc4)CC3)CC2)nc1)C1CCC(=O)NC1=O] |
| 182 | 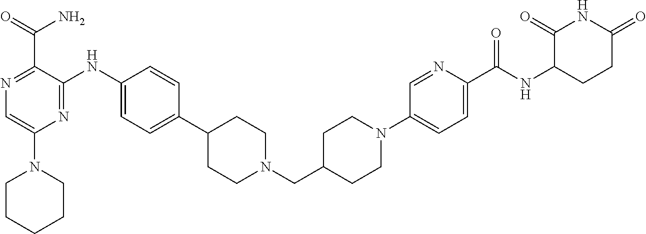 [SMILES: NC(=O)c1ncc(N2CCCCC2)nc1Nc1ccc(C2CCN(CC3CCN(c4ccc(C(=O)NC5CCC(=O)NC5=O)nc4)CC3)CC2)cc1] |
| 183 | 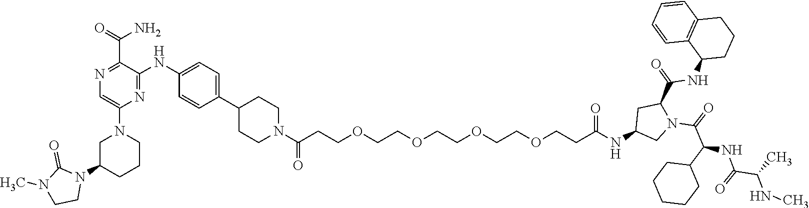 |
| 184 | 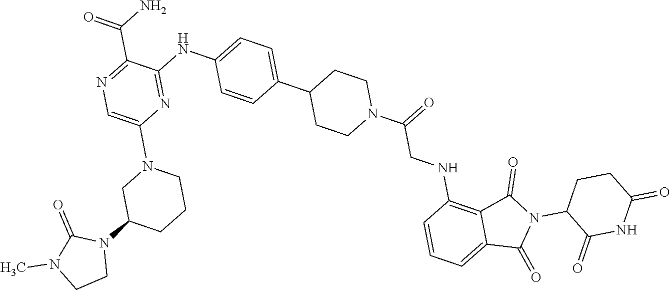 [SMILES: CN1CCN([C@@H]2CCCN(c3cnc(C(N)=O)c(Nc4ccc(C5CCN(C(=O)CNc6cccc7c6C(=O)N(C6CCC(=O)NC6=O)C7=O)CC5)cc4)n3)C2)C1=O] |
| 185 | 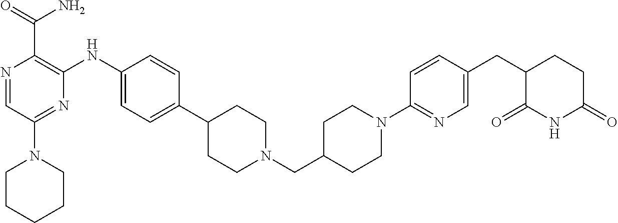 [SMILES: NC(=O)c1ncc(N2CCCCC2)nc1Nc1ccc(C2CCN(CC3CCN(c4ccc(CC5CCC(=O)NC5=O)cn4)CC3)CC2)cc1] |
| 186 | 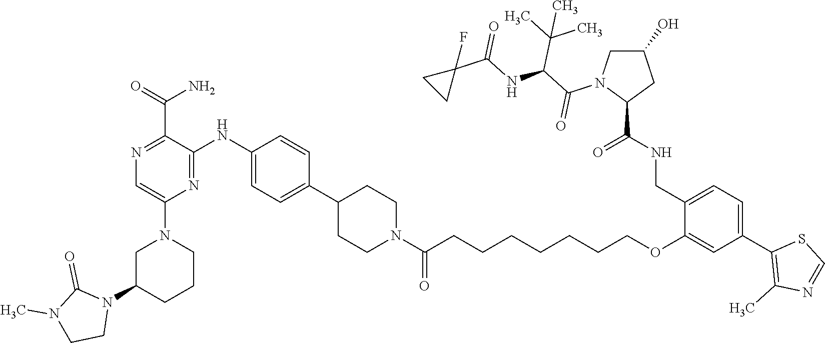 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCC(=O)N2CCC(c3ccc(Nc4nc(N5CCC[C@@H](N6CCN(C)C6=O)C5)cnc4C(N)=O)cc3)CC2)c1] |
| 187 | 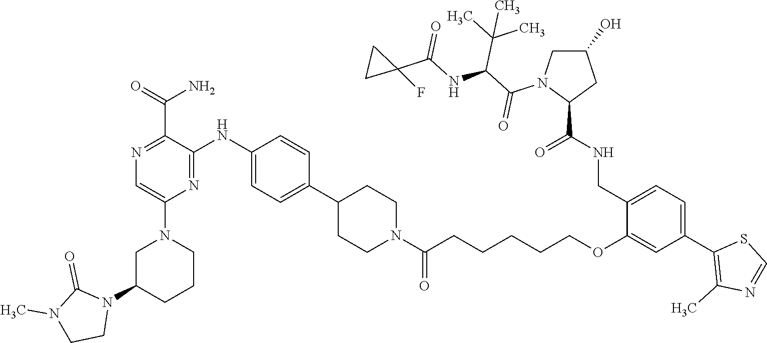 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCC(=O)N2CCC(c3ccc(Nc4nc(N5CCC[C@@H](N6CCN(C)C6=O)C5)cnc4C(N)=O)cc3)CC2)c1] |
| 188 | 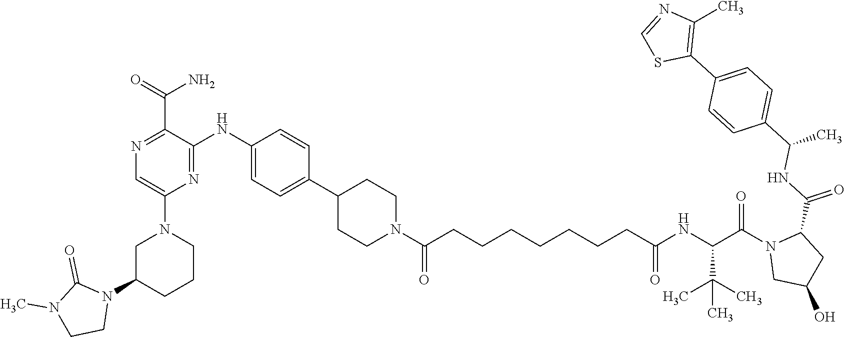 [SMILES: Cc1ncsc1-c1ccc([C@H](C)NC(=O)[C@@H]2C[C@@H](O)CN2C(=O)[C@@H](NC(=O)CCCCCCCC(=O)N2CCC(c3ccc(Nc4nc(N5CCC[C@@H](N6CCN(C)C6=O)C5)cnc4C(N)=O)cc3)CC2)C(C)(C)C)cc1] |
| 189 | 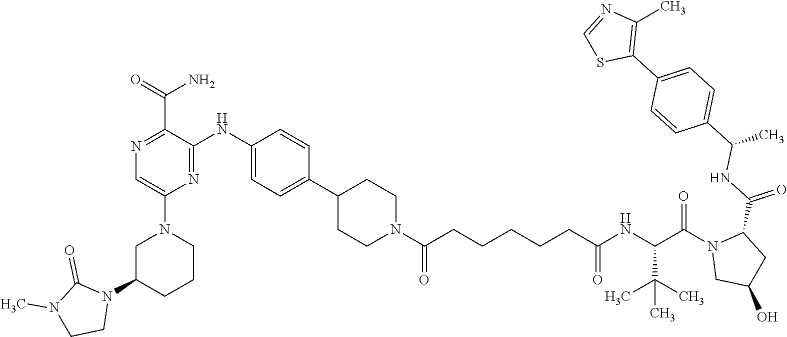 [SMILES: Cc1ncsc1-c1ccc([C@H](C)NC(=O)[C@@H]2C[C@@H](O)CN2C(=O)[C@@H](NC(=O)CCCCCC(=O)N2CCC(c3ccc(Nc4nc(N5CCC[C@@H](N6CCN(C)C6=O)C5)cnc4C(N)=O)cc3)CC2)C(C)(C)C)cc1] |
| 190 | 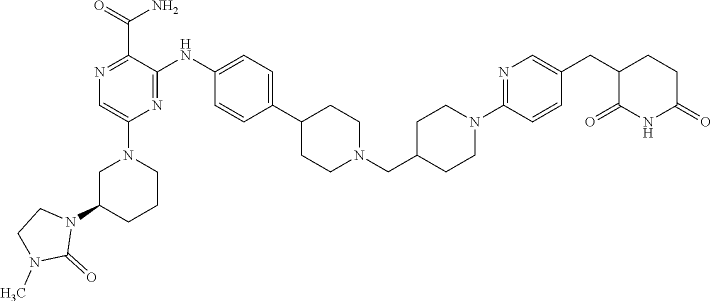 [SMILES: CN1CCN([C@@H]2CCCN(c3cnc(C(N)=O)c(Nc4ccc(C5CCN(CC6CCN(c7ccc(CC8CCC(=O)NC8=O)cn7)CC6)CC5)cc4)n3)C2)C1=O] |
| 191 | 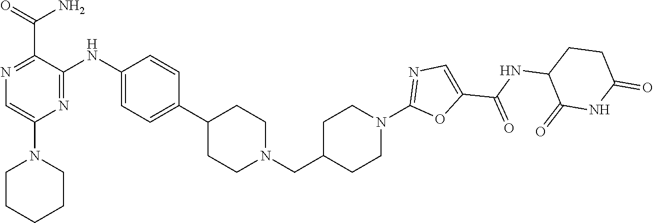 [SMILES: NC(=O)c1ncc(N2CCCCC2)nc1Nc1ccc(C2CCN(CC3CCN(c4ncc(C(=O)NC5CCC(=O)NC5=O)o4)CC3)CC2)cc1] |
| 192 | 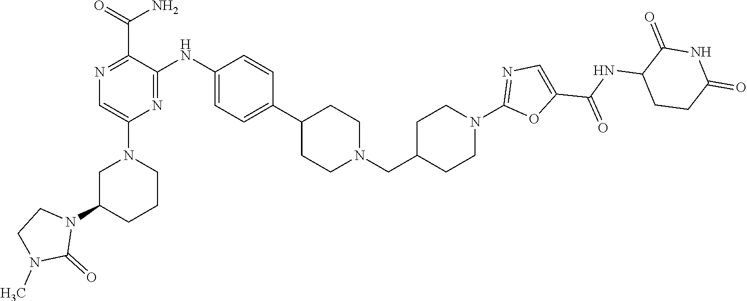 [SMILES: CN1CCN([C@@H]2CCCN(c3cnc(C(N)=O)c(Nc4ccc(C5CCN(CC6CCN(c7ncc(C(=O)NC8CCC(=O)NC8=O)o7)CC6)CC5)cc4)n3)C2)C1=O] |
| 193 | 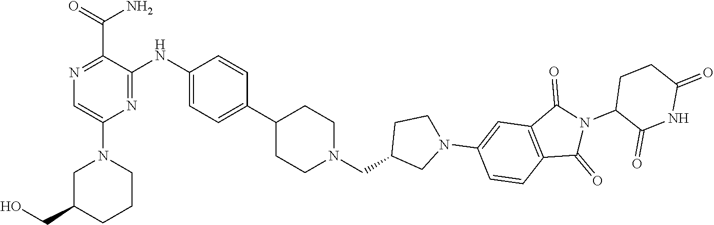 [SMILES: NC(=O)c1ncc(N2CCC[C@@H](CO)C2)nc1Nc1ccc(C2CCN(C[C@@H]3CCN(c4ccc5c(c4)C(=O)N(C4CCC(=O)NC4=O)C5=O)C3)CC2)cc1] |

### III. Uses, Formulations, and Administration

A. Pharmaceutical Compositions

The compounds described herein can be formulated into pharmaceutical compositions that further comprise a pharmaceutically acceptable carrier, diluent, adjuvant or vehicle. In one embodiment, the present invention provides a pharmaceutical composition comprising a compound of the invention described above, and a pharmaceutically acceptable carrier, diluent, adjuvant or vehicle. In one embodiment, the present invention is a pharmaceutical composition comprising an effective amount of a compound of the present invention or a pharmaceutically acceptable salt thereof and a pharmaceutically acceptable carrier, diluent, adjuvant or vehicle. Pharmaceutically acceptable carriers include, for example, pharmaceutical diluents, excipients or carriers suitably selected with respect to the intended form of administration, and consistent with conventional pharmaceutical practices.

According to another embodiment, the invention provides a composition comprising a compound of this invention or a pharmaceutically acceptable salt thereof and a pharmaceutically acceptable carrier, adjuvant, or vehicle. Pharmaceutical compositions of this invention comprise a therapeutically effective amount of a compound of Formula (A), (B), (C), (D), (E), (F), (G), (H), (J), (K), (M), (I) (II) (III) and/or (X) wherein a “therapeutically effective amount” is an amount that is (a) effective to measurably degrade BTK (or reduce the amount of BTK) in a biological sample or in a patient, or (b) effective in treating and/or ameliorating a disease or disorder that is mediated by BTK.

The term “patient,” as used herein, means an animal, preferably a mammal, and most preferably a human.

It also will be appreciated that certain of the compounds of the present invention can exist in free form for treatment, or where appropriate, as a pharmaceutically acceptable derivative (e.g., a salt) thereof. According to the present invention, a pharmaceutically acceptable derivative includes, but is not limited to, pharmaceutically acceptable prodrugs, salts, esters, salts of such esters, or any other adduct or derivative that upon administration to a patient in need is capable of providing, directly or indirectly, a compound as otherwise described herein, or a metabolite or residue thereof.

As used herein, the term “pharmaceutically acceptable salt” refers to those salts that are, within the scope of sound medical judgement, suitable for use in contact with the tissues of humans and lower animals without undue toxicity, irritation, allergic response and the like.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts include salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N+(C₁₋₄alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

A pharmaceutically acceptable carrier may contain inert ingredients that do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

The pharmaceutically acceptable carrier, adjuvant, or vehicle, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds described herein, such as by producing any undesirable biological effect or otherwise interacting in a deleterious manner with any other component(s) of the pharmaceutically acceptable composition, the use of such conventional carrier medium is contemplated to be within the scope of this invention. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful, uncomfortable, or risky. Side effects include, but are not limited to, fever, chills, lethargy, gastrointestinal toxicities (including gastric and intestinal ulcerations and erosions), nausea, vomiting, neurotoxicities, nephrotoxicities, renal toxicities (including such conditions as papillary necrosis and chronic interstitial nephritis), hepatic toxicities (including elevated serum liver enzyme levels), myelotoxicities (including leukopenia, myelosuppression, thrombocytopenia and anemia), dry mouth, metallic taste, prolongation of gestation, weakness, somnolence, pain (including muscle pain, bone pain and headache), hair loss, asthenia, dizziness, extra-pyramidal symptoms, akathisia, cardiovascular disturbances and sexual dysfunction.

Some examples of materials that can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents. Preservatives and antioxidants can also be present in the composition, according to the judgment of the formulator.

As used herein, the term “measurably degrade,” means a measurable reduction in (a) BTK activity, between a sample comprising a compound of this invention and a BTK and an equivalent sample comprising a BTK in the absence of said compound, or (b) the concentration of the BTK in a sample over time.

The compositions of the present invention may be administered orally, parenterally, by inhalation spray, topically, rectally, nasally, buccally, vaginally or via an implanted reservoir. As used herein, the term “parenteral” includes subcutaneous, intravenous, intramuscular, intra-articular, intra-synovial, intrasternal, intrathecal, intraocular, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation also may be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives, are useful in the preparation of injectables, as are natural pharmaceutically acceptable oils, such as olive oil or castor oil, especially in their polyoxyethylated versions. These oil solutions or suspensions also may contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers that are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents also may be added.

Alternatively, the pharmaceutically acceptable compositions of this invention may be administered in the form of suppositories for rectal or vaginal administration. These can be prepared by mixing the agent with a suitable non-irritating excipient that is solid at room temperature but liquid at rectal temperature and therefore will melt in the rectum or vaginal cavity to release the drug. Such materials include cocoa butter, polyethylene glycol or a suppository wax that is solid at ambient temperature but liquid at body temperature and therefore melt in the rectum or vaginal cavity and release the active compound.

The pharmaceutically acceptable compositions of this invention also may be administered topically, especially when the target of treatment includes areas or organs readily accessible by topical application, including diseases of the eye, skin, or lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches also may be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated, e.g., as micronized suspensions in isotonic, pH adjusted sterile saline or other aqueous solution, or, preferably, as solutions in isotonic, pH adjusted sterile saline or other aqueous solution, either with or without a preservative such as benzylalkonium chloride. Alternatively, for ophthalmic uses, the pharmaceutically acceptable compositions may be formulated in an ointment such as petrolatum. The pharmaceutically acceptable compositions of this invention also may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions also can include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation also may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid may be used in the preparation of injectables.

The injectable formulations can be sterilized, for example, by filtration through a bacterial-retaining filter, or by incorporating sterilizing agents in the form of sterile solid compositions that can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations also are prepared by entrapping the compound in liposomes or microemulsions that are compatible with body tissues.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form also may comprise buffering agents.

Solid compositions of a similar type also may be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. Solid dosage forms optionally may contain opacifying agents. These solid dosage forms also can be of a composition such that they release the active ingredient(s) only, or preferentially, in a certain part of the intestinal tract, optionally, in a delayed manner. Examples of embedding compositions that can be used include polymeric substances and waxes. Solid compositions of a similar type also may be employed as fillers in soft and hard-filled gelatin capsules using such excipients as lactose or milk sugar as well as high molecular weight polethylene glycols and the like.

The active compounds also can be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms also may comprise, as is normal practice, additional substances other than inert diluents, e.g., tableting lubricants and other tableting aids such a magnesium stearate and microcrystalline cellulose. In the case of capsules, tablets and pills, the dosage forms also may comprise buffering agents. They may optionally contain opacifying agents and also can be of a composition such that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops also are contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers also can be used to increase the flux of the compound across the skin. The rate can be controlled by either providing a rate controlling membrane or by dispersing the compound in a polymer matrix or gel.

The compounds of the invention preferably are formulated in dosage unit form for ease of administration and uniformity of dosage. As used herein, the phrase “dosage unit form” refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

The amount of the compounds of the present invention that may be combined with the carrier materials to produce a composition in a single dosage form will vary depending upon the host treated, the particular mode of administration, and other factors. Preferably, the compositions should be formulated so that a dosage of between 0.01-100 mg/kg body weight/day of the inhibitor can be administered to a patient receiving these compositions.

Depending upon the particular condition, or disease, to be treated or prevented, additional therapeutic agents, which are normally administered to treat or prevent that condition, also may be present in the compositions of this invention. As used herein, additional therapeutic agents that are normally administered to treat or prevent a particular disease, or condition, are known as “appropriate for the disease, or condition, being treated.”

For example, chemotherapeutic agents or other anti-proliferative agents may be combined with the compounds of this invention to treat proliferative diseases and cancer. Examples of known chemotherapeutic agents include, but are not limited to, PI3K inhibitors (e.g., idelalisib and copanlisib), BCL-2 inhibitors (e.g., venetoclax), BTK inhibitors (e.g., ibrutinib and acalabrutinib), etoposide, CD20 antibodies (e.g., rituximab, ocrelizumab, obinutuzumab, ofatumumab, ibritumomab tiuxetan, tositumomab, and ublituximab), aletuzumab, bendamustine, cladribine, doxorubicin, chlorambucil, prednisone, midostaurin, lenalidomide, pomalidomide, checkpoint inhibitors (e.g., ipilimumab, nivolumab, pembolizumab, atezolizumab, avelumab, durvalumab), engineered cell therapy (e.g., CAR-T therapy—Kymriah®, Yescarta®), Gleevec™, adriamycin, dexamethasone, vincristine, cyclophosphamide, fluorouracil, topotecan, taxol, interferons, and platinum derivatives.

And, in some instances, radiation therapy is administered during the treatment course wherein a compound of the present invention (or a pharmaceutically acceptable salt thereof) is administered to a patient in need thereof.

Other examples of agents with which the inhibitors of this invention also may be combined include, without limitation: treatments for Alzheimer's Disease such as Aricept® and Excelon®; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif*), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; and agents for treating immunodeficiency disorders such as gamma globulin.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

B. Uses of the Compounds and Compositions.

The bifunctional compounds of the present invention are useful for degrading BTK in biological samples or in patients via a ubiquitin proteolytic pathway. Thus, an embodiment of the present invention provides a method of treating a BTK-mediated disease or disorder. As used herein, the term “BTK-mediated disease or disorder” means any disease, disorder, or other deleterious condition in which a BTK is known to play a role. In some instances, a BTK-mediated disease or disorder is a proliferative disorder or an autoimmune disorder. Examples of proliferative disorders include cancer.

The term “cancer” includes, but is not limited to, the following cancers: epidermoid Oral: buccal cavity, lip, tongue, mouth, pharynx; Cardiac: sarcoma (angiosarcoma, fibrosarcoma, rhabdomyosarcoma, liposarcoma), myxoma, rhabdomyoma, fibroma, lipoma, and teratoma; Lung: bronchogenic carcinoma (squamous cell or epidermoid, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, rectum; Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma) hairy cell; lymphoid disorders (e.g., mantle cell lymphoma, Waldenström's macroglobulinemia, Marginal zone lymphoma, and Follicular lymphoma); Skin: malilymphgnant melanoma, basal cell carcinoma, squamous cell carcinoma, Karposi's sarcoma, keratoacanthoma, moles dysplastic nevi, lipoma, angioma, dermatofibroma, keloids, psoriasis, Thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; and Adrenal glands: neuroblastoma.

Examples of autoimmune disorders include uticaria, graft-versus-host disease, pemphigus vulgaris, achalasia, Addison's disease, Adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease (AIED), autoimmune myocarditis, autoimmune oophoritis, autoimmune orchitis, autoimmune pancreatitis, autoimmune retinopathy, axonal and neuronal neuropathy (AMAN), Baló disease, Behcet's disease, benign mucosal pemphigoid, bullous pemphigoid, Castleman disease (CD), Celiac disease, Chagas disease, chronic inflammatory demyelinating polyneuropathy (CIDP), chronic recurrent multifocal osteomyelitis (CRMO), Churg-Strauss Syndrome (CSS) or Eosinophilic Granulomatosis (EGPA), cicatricial pemphigoid, Cogan's syndrome, cold agglutinin disease, congenital heart block, coxsackie myocarditis, CREST syndrome, Crohn's disease, dermatitis herpetiformis, dermatomyositis, Devic's disease (neuromyelitis optica), discoid lupus, Dressler's syndrome, endometriosis, eosinophilic esophagitis (EoE), eosinophilic fasciitis, erythema nodosum, essential mixed cryoglobulinemia, evans syndrome, fibromyalgia, fibrosing alveolitis, giant cell arteritis (temporal arteritis), giant cell myocarditis, glomerulonephritis, goodpasture's syndrome, granulomatosis with polyangiitis, Graves' disease, Guillain-Barre syndrome, Hashimoto's thyroiditis, hemolytic anemia, Henoch-Schonlein purpura (HSP), herpes gestationis or pemphigoid gestationis (PG), hidradenitis suppurativa (HS) (Acne Inversa), hypogammalglobulinemia, IgA nephropathy, IgG4-related sclerosing disease, immune thrombocytopenic purpura (ITP), inclusion body myositis (IBM), interstitial cystitis (IC), juvenile arthritis, juvenile diabetes (Type 1 diabetes), juvenile myositis (JM), Kawasaki disease, Lambert-Eaton syndrome, leukocytoclastic vasculitis, lichen planus, lichen sclerosus, ligneous conjunctivitis, linear IgA disease (LAD), lupus, lyme disease chronic, Meniere's disease, microscopic polyangiitis (MPA), mixed connective tissue disease (MCTD), Mooren's ulcer, Mucha-Habermann disease, Multifocal Motor Neuropathy (MMN) or MMNCB, multiple sclerosis, myasthenia gravis, myositis, narcolepsy, neonatal lupus, neuromyelitis optica, neutropenia, ocular cicatricial pemphigoid, optic neuritis, palindromic rheumatism (PR), PANDAS, paraneoplastic cerebellar degeneration (PCD), paroxysmal nocturnal hemoglobinuria (PNH), Parry Romberg syndrome, pars planitis (peripheral uveitis), Parsonnage-Turner syndrome, pemphigus, peripheral neuropathy, perivenous encephalomyelitis, pernicious anemia (PA), POEMS syndrome, polyarteritis nodosa, polyglandular syndromes type I, II, III, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia (PRCA), pyoderma gangrenosum, Raynaud's phenomenon, reactive Arthritis, reflex sympathetic dystrophy, relapsing polychondritis, restless legs syndrome (RLS), retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome (SPS), subacute bacterial endocarditis (SBE), Susac's syndrome, sympathetic ophthalmia (SO), Takayasu's arteritis, temporal arteritis (giant cell arteritis), thrombocytopenic purpura (TTP), Tolosa-Hunt syndrome (THS), transverse myelitis, Type 1 diabetes, ulcerative colitis (UC), undifferentiated connective tissue disease (UCTD), uveitis, vasculitis, vitiligo, Vogt-Koyanagi-Harada Disease, and Wegener's granulomatosis (or Granulomatosis with Polyangiitis (GPA)).

### IV. EXAMPLES

Additional embodiments are disclosed in further detail in the following examples, which are not in any way intended to limit the scope of the claims.

Preliminary Synthesis.

### Step 1: Synthesis of 2-(2,6-dioxopiperidin-3-yl)-5-fluoroisoindoline-1,3-dione

A mixture of 5-fluoro-1,3-dihydro-2-benzofuran-1,3-dione (5.0 g, 30.10 mmol), 3-aminopiperidine-2,6-dione hydrochloride (6.9 g, 42.14 mmol) and NaOAc (4.2 g, 51.17 mmol) in HOAc (50 mL) was stirred at 120° C. for 5 h before concentrating under vacuum. The residue was washed with water and the solid was collected by filtration. The crude product was washed with water twice and ethyl acetate twice and dried to afford 2-(2,6-dioxopiperidin-3-yl)-5-fluoroisoindoline-1,3-dione (7.7 g, 92%) as a light brown solid. H NMR (300 MHz, DMSO-d₆) δ 11.16 (s, 1H), 8.03-8.00 (m, 1H), 7.87-7.85 (m, 1H), 7.75-7.70 (m, 1H), 5.19-5.15 (m, 1H), 2.94-2.86 (m, 1H), 2.63-2.48 (m, 2H), 2.12-2.06 (m, 1H). F NMR (300 MHz, DMSO-d₆) δ-102.078.

### Step 2: Amine Displacement of Aryl Fluoride

To a solution of 2-(2,6-dioxopiperidin-3-yl)-5-fluoro-2,3-dihydro-1H-isoindole-1,3-dione (1.0 g, 3.62 mmol) in N-Methyl pyrrolidone (10 mL) were added the amine (3.60 mmol) and DIEA (1.4 g, 10.83 mmol). The resulting solution was stirred at 80° C. for 16 h. The reaction mixture was cooled down to room temperature and purified by reverse phase flash chromatography to afford the corresponding final product.

### Step 3: Alcohol Oxidation to Aldehyde

To a mixture of alcohol (1.06 mmol) in CH₂Cl₂ (10 mL) was added Dess-Martin periodinane (2.12 mmol). The mixture was allowed to stir at room temperature for 1 h. The mixture was purified by column chromatography to afford the desired aldehyde.

### Example 1: Synthesis of 5-[(3R)-3-(3-methyl-2-oxoimidazolidin-1-yl)piperidin-1-yl]-3-{[4-(piperazin-1-yl)phenyl]amino}pyrazine-2-carboxamide

Step 1: tert-butyl (3R)-3-{[(2-chloroethyl)carbamoyl]amino}piperidine-1-carboxylate.

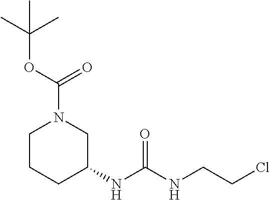 [SMILES: CC(C)(C)OC(=O)N1CCC[C@@H](NC(=O)NCCCl)C1]

To a mixture of tert-butyl (3R)-3-aminopiperidine-1-carboxylate (25.0 g, 125 mmol) and triethylamine (34.8 mL, 25.3 g, 250 mmol) in DCM (250 mL) was added 1-chloro-2-isocyanatoethane (12.8 mL, 15.8 g, 150 mmol) over 25 minutes. A mild exotherm was observed. After 4 hours, 100 mL water was added. The layers were separated. The organic layer was dried over Na₂SO₄ and concentrated. The mixture was dissolved in ethyl acetate and filtered through 1000 cc of silica gel in a 2000 mL buchner funnel eluted with ethyl acetate. The resulting solution was concentrated in vacuo to provide tert-butyl (3R)-3-{[(2-chloroethyl)carbamoyl]amino}piperidine-1-carboxylate (40.6 g, quant) which was used without further purification. LCMS: C₁₃H₂₄ClN₃O₃ requires 305, found: m/z=306 [M+H]⁺.

### Step 2: tert-butyl (3R)-3-(2-oxoimidazolidin-1-yl)piperidine-1-carboxylate

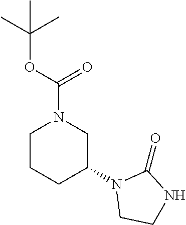 [SMILES: CC(C)(C)OC(=O)N1CCC[C@@H](N2CCNC2=O)C1]

To an ice cooled mixture of tert-butyl (3R)-3-{[(2-chloroethyl)carbamoyl]amino}piperidine-1-carboxylate (40.3 g, 132 mmol) in THE (400 mL) was added 60% sodium hydride (10.6 g, 264 mmol) in portions. The cooling bath was allowed to melt and the reaction was stirred at room temp overnight. Another portion of 60% sodium hydride (5.65 g, 141 mmol) was added, causing gas evolution. After ten minutes, a mild exotherm was observed. After 2 hours, the reaction was quenched by the addition of 75 mL water. The layers were separated. The aqueous layer was extracted with two 50 mL portions of DCM. The combined organic layers were washed with brine, dried over anhydrous Na₂SO₄, and concentrated in vacuo. The resulting material was partitioned between acetonitrile and hexanes. The acetonitrile layer was concentrated in vacuo to provide tert-butyl (3R)-3-(2-oxoimidazolidin-1-yl)piperidine-1-carboxylate (33.9 g, 95.4%). LCMS: C₁₃H₂₃N₃O₃ requires 269, found: m/z=270 [M+H]⁺.

Step 3: tert-butyl (3R)-3-(2-oxoimidazolidin-1-yl)piperidine-1-carboxylate

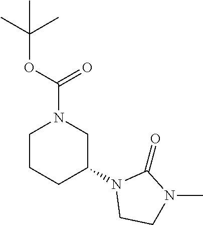 [SMILES: CN1CCN([C@@H]2CCCN(C(=O)OC(C)(C)C)C2)C1=O]

To an ice cooled mixture of tert-butyl (3R)-3-(2-oxoimidazolidin-1-yl)piperidine-1-carboxylate (33.8 g, 126 mmol) in THE (300 mL) was added 60% sodium hydride (10.1 g, 251 mmol) in portions. After 5 minutes, the cooling bath was removed and gas evolution was observed for 1 hour. The mixture was cooled in an ice bath. Methyl iodide (11.7 mL, 26.7 g, 188 mmol) was added over 5 minutes. The cooling bath was allowed to expire. After stirring for 16 hours at room temperature, the reaction was quenched with 75 mL water. The layers were separated. The organic layer was washed with brine. The combined aqueous layers were extracted twice with DCM. The combined organic layers were dried over anhydrous Na₂SO₄ and concentrated. The resulting material was partitioned between acetonitrile and hexane. The acetonitrile layer was filtered and concentrated in vacuo to provide tert-butyl (3R)-3-(3-methyl-2-oxoimidazolidin-1-yl)piperidine-1-carboxylate (38.4 g, quant) which was used crude without further purification. LCMS: C₁₄H₂₅N₃O₃ requires 283, found: m/z=306 [M+Na]⁺.

### Step 4: 1-methyl-3-[(3R)-piperidin-3-yl]imidazolidin-2-one hydrochloride

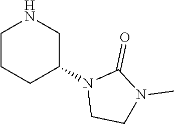

Tert-butyl (3R)-3-(3-methyl-2-oxoimidazolidin-1-yl)piperidine-1-carboxylate (35.1 g, 124 mmol) was stirred in hydrogen chloride 4M solution in dioxane (310 mL, 1.24 mol) for 2 hours. The mixture was concentrated in vacuo to provide 1-methyl-3-[(3R)-piperidin-3-yl]imidazolidin-2-one hydrochloride (35.0 g, quant) which was used crude without further purification. LCMS: C₉H₁₇N₃O requires 183, found: m/z=184 [M+H]⁺.

### Step 5: 3-chloro-5-[(3R)-3-(3-methyl-2-oxoimidazolidin-1-yl)piperidin-1-yl]pyrazine-2-carbonitrile

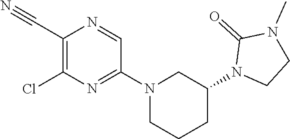 [SMILES: CN1CCN([C@@H]2CCCN(c3cnc(C#N)c(Cl)n3)C2)C1=O]

3,5-dichloropyrazine-2-carbonitrile (21.6 g, 124 mmol) was added to an ice cooled mixture of 1-methyl-3-[(3R)-piperidin-3-yl]imidazolidin-2-one hydrochloride (27.2 g, 1 24 mmol) and N,N-diisopropylethylamine (86.3 mL, 495 mmol) in DMF (300 mL). After 15 minutes, the cooling bath was removed. After stirring for 16 hours, the mixture was diluted with 800 mL water. The mixture was extracted with ethyl acetate. The organic layer was washed twice with water and washed once with brine. The organic layer was dried over anhydrous Na₂SO₄ and concentrated in vacuo. The crude residue was purified by flash chromatography on a 330 g silica gel column eluted with 0 to 3% MeOH/DCM gradient to provide 3-chloro-5-[(3R)-3-(3-methyl-2-oxoimidazolidin-1-yl)piperidin-1-yl]pyrazine-2-carbonitrile (22.1 g, 55.6%). LCMS: C14H₁₇ClN6O requires 320, found: m/z=320 [M+H]⁺.

### Step 6: tert-butyl 4-[4-({3-cyano-6-[(3R)-3-(3-methyl-2-oxoimidazolidin-1-yl)piperidin-1-yl]pyrazin-2-yl}amino)phenyl]piperazine-1-carboxylate

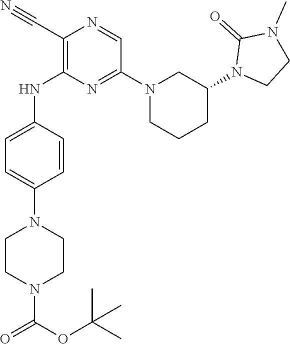 [SMILES: CN1CCN([C@@H]2CCCN(c3cnc(C#N)c(Nc4ccc(N5CCN(C(=O)OC(C)(C)C)CC5)cc4)n3)C2)C1=O]

3-chloro-5-[(3R)-3-(3-methyl-2-oxoimidazolidin-1-yl)piperidin-1-yl]pyrazine-2-carbonitrile (9.57 g, 29.8 mmol), tert-butyl 4-(4-aminophenyl)piperazine-1-carboxylate (8.27 g, 29.8 mmol), and cesium carbonate (29.2 g, 89.5 mmol) were deposited in a 200 mL round bottom flask with dioxane (75 mL). A vacuum was pulled on the flask until the mixture bubbled and the headspace was backfilled with argon for 5 cycles. BINAP (1.86 g, 2.98 mmol) and palladium (II) acetate (670 mg, 2.98 mmol) were added. A vacuum was pulled on the flask and the headspace was backfilled with argon for 5 cycles. The mixture was heated at 100° C. for 3 hours. The mixture was filtered. The solid was washed with DCM. The resulting solution was concentrated in vacuo. The crude residue was purified by flash chromatography on a 330 g silica gel column eluted with 0 to 5% MeOH/DCM gradient to provide tert-butyl 4-[4-({3-cyano-6-[(3R)-3-(3-methyl-2-oxoimidazolidin-1-yl)piperidin-1-yl]pyrazin-2-yl}amino)phenyl]piperazine-1-carboxylate (8.81 g, 52.6%). LCMS: C₂₉H₃₉N₉O₃ requires 561, found: m/z=584 [M+Na]+.

### Step 7: tert-butyl 4-[4-({3-carbamoyl-6-[(3R)-3-(3-methyl-2-oxoimidazolidin-1-yl)piperidin-1-yl]pyrazin-2-yl}amino)phenyl]piperazine-1-carboxylate

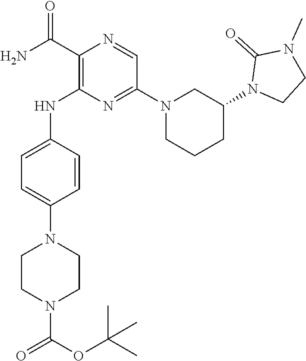 [SMILES: CN1CCN([C@@H]2CCCN(c3cnc(C(N)=O)c(Nc4ccc(N5CCN(C(=O)OC(C)(C)C)CC5)cc4)n3)C2)C1=O]

To a homogeneous solution of tert-butyl 4-[4-({3-cyano-6-[(3R)-3-(3-methyl-2-oxoimidazolidin-1-yl)piperidin-1-yl]pyrazin-2-yl}amino)phenyl]piperazine-1-carboxylate (8.23 g, 14.7 mmol) in DMSO (80 mL) and MeOH (160 mL) was added cesium carbonate (4.77 g, 14.7 mmol). The mixture was cooled in an ice bath. Hydrogen peroxide 30% solution (22.0 mL, 213 mmol) was added in 2 portions. After 5 minutes, the ice bath was removed. After 2 hours at room temperature, the mixture was cooled in an ice bath. 70 mL acetonitrile was added. The ice bath was removed. After 15 minutes, the volatiles were removed in vacuo and the mixture was diluted with 1 L of ethyl acetate. The mixture was washed with three portions of water then washed with brine. The organic layer was dried over anhydrous Na₂SO₄ and concentrated in vacuo. The crude residue was purified by flash chromatography on a 220 g silica gel column eluted with 0 to 10% MeOH/EtOAc gradient to provide tert-butyl 4-[4-({3-carbamoyl-6-[(3R)-3-(3-methyl-2-oxoimidazolidin-1-yl)piperidin-1-yl]pyrazin-2-yl}amino)phenyl]piperazine-1-carboxylate (7.23 g, 85.1%). LCMS: C₂₉H₄₁N₉O₄ requires 579, found m/z=580 [M+H]⁺.

### Step 8: 5-[(3R)-3-(3-methyl-2-oxoimidazolidin-1-yl)piperidin-1-yl]-3-{[4-(piperazin-1-yl)phenyl]amino}pyrazine-2-carboxamide trifluoroacetate

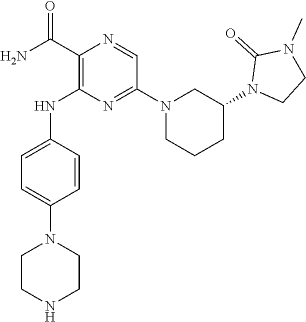 [SMILES: CN1CCN([C@@H]2CCCN(c3cnc(C(N)=O)c(Nc4ccc(N5CCNCC5)cc4)n3)C2)C1=O]

Tert-butyl 4-[4-({3-carbamoyl-6-[(3R)-3-(3-methyl-2-oxoimidazolidin-1-yl)piperidin-1-yl]pyrazin-2-yl}amino)phenyl]piperazine-1-carboxylate (2.65 g, 4.57 mmol) was stirred in DCM (15 mL) and TFA (15 mL). After 30 minutes, the mixture was concentrated to provide 5-[(3R)-3-(3-methyl-2-oxoimidazolidin-1-yl)piperidin-1-yl]-3-{[4-(piperazin-1-yl)phenyl]amino}pyrazine-2-carboxamide trifluoroacetate (2.71 g, 100%). LCMS: C₂₄H₃₃N₉O₂ requires 479, found m/z=480 [M+H]⁺.

### Step 9: tert-butyl 4-[4-({3-cyano-6-[(3R)-3-(3-methyl-2-oxoimidazolidin-1-yl)piperidin-1-yl]pyrazin-2-yl}amino)phenyl]piperidine-1-carboxylate

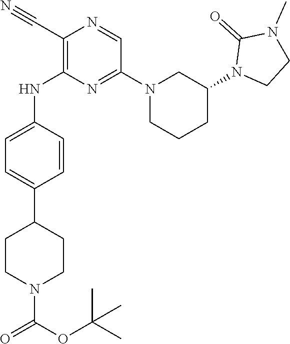 [SMILES: CN1CCN([C@@H]2CCCN(c3cnc(C#N)c(Nc4ccc(C5CCN(C(=O)OC(C)(C)C)CC5)cc4)n3)C2)C1=O]

A mixture of 3-chloro-5-[(3R)-3-(3-methyl-2-oxoimidazolidin-1-yl)piperidin-1-yl]pyrazine-2-carbonitrile (244 mg, 0.76 mmol), tert-butyl 4-(4-aminophenyl)piperidine-1-carboxylate (211 mg, 0.76 mmol), Pd(OAc)₂ (56.4 mg, 0.25 mmol), BINAP (156.3 mg, 0.25 mmol) and Cs₂CO₃ (7434 mg, 2.28 mmol) was degassed and backfilled with N₂ 5 times. The mixture was allowed to stir at 100° C. for 90 min. The mixture was filtered through celite washing with MeOH/EtOAc, concentrated and purified by MPLC (0-100% EtOAc in CH₂Cl₂) to afford tert-butyl 4-[4-({3-cyano-6-[(3R)-3-(3-methyl-2-oxoimidazolidin-1-yl)piperidin-1-yl]pyrazin-2-yl}amino)phenyl]piperidine-1-carboxylate (259 mg, 60.7%). LCMS: C₃₀H₄₀N₈O₃ requires 560, found m/z=561 [M+H]⁺.

### Step 10: tert-butyl 4-[4-({3-carbamoyl-6-[(3R)-3-(3-methyl-2-oxoimidazolidin-1-yl)piperidin-1-yl]pyrazin-2-yl}amino)phenyl]piperidine-1-carboxylate

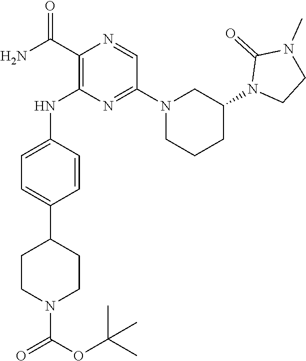 [SMILES: CN1CCN([C@@H]2CCCN(c3cnc(C(N)=O)c(Nc4ccc(C5CCN(C(=O)OC(C)(C)C)CC5)cc4)n3)C2)C1=O]

H₂O₂(30% in water, 2.50 mL, 0.24 mmol) was added to a mixture of tert-butyl 4-[4-({3-cyano-6-[(3R)-3-(3-methyl-2-oxoimidazolidin-1-yl)piperidin-1-yl]pyrazin-2-yl}amino)phenyl]piperidine-1-carboxylate (259 mg, 0.46 mmol), Cs₂CO₃ (150.5 mg, 0.46 mmol), MeOH (9 mL) and DMSO (0.5 mL). The mixture was allowed to stir at rt for 30 min. The mixture was concentrated, EtOAc was added and the organic phase was washed with H₂O and brine. The organic layer was dried with MgSO₄, filtered, concentrated and purified by MPLC (0-10% MeOH in CH₂Cl₂) to afford tert-butyl 4-[4-({3-carbamoyl-6-[(3R)-3-(3-methyl-2-oxoimidazolidin-1-yl)piperidin-1-yl]pyrazin-2-yl}amino)phenyl]piperidine-1-carboxylate (252 mg, 94%). LCMS: C₃₀H₄₂N₈O₄ requires 578, found m/z=579 [M+H]⁺.

### Step 11: 5-[(3R)-3-(3-methyl-2-oxoimidazolidin-1-yl)piperidin-1-yl]-3-f{[4-(piperidin-4-yl)phenyl]amino}pyrazine-2-carboxamide

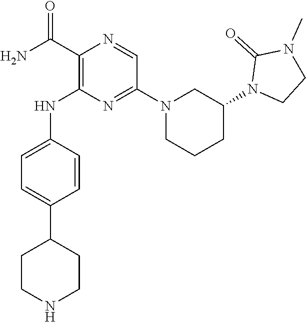 [SMILES: CN1CCN([C@@H]2CCCN(c3cnc(C(N)=O)c(Nc4ccc(C5CCNCC5)cc4)n3)C2)C1=O]

A mixture of tert-butyl 4-[4-({3-carbamoyl-6-[(3R)-3-(3-methyl-2-oxoimidazolidin-1-yl)piperidin-1-yl]pyrazin-2-yl}amino)phenyl]piperidine-1-carboxylate (252 mg, 0.44 mmol), hydrogen chloride (4M in dioxane, 2.72 mL, 10.89 mmol) and THE (2 mL) was allowed to stir at r.t. for 2 h. The volatiles were removed to afford 5-[(3R)-3-(3-methyl-2-oxoimidazolidin-1-yl)piperidin-1-yl]-3-{[4-(piperidin-4-yl)phenyl]amino}pyrazine-2-carboxamide (209 mg, quant).

### Example 2: 5-[(3R)-3-(3-methyl-2-oxoimidazolidin-1-yl)piperidin-1-yl]-3-{[4-(4-methylpiperidin-4-yl)phenyl]amino}pyrazine-2-carboxamide

### Step 1: ethyl 4-hydroxy-4-methylpiperidine-1-carboxylate

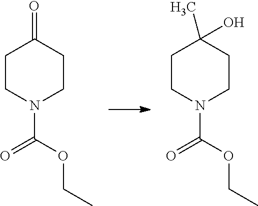

A solution of ethyl 4-oxopiperidine-1-carboxylate (10.00 g, 58.41 mmol) in diethyl ether (100.00 mL) was cooled to −30° C., chloro(methyl)magnesium (23.40 mL, 5.24 g, 70.10 mmol) (3M solution in THF) was added. The resulting mixture was stirred at 0° C. for 2 hrs, and TLC showed no starting material. The reaction was quenched with 50 mL ammonium chloride solution and a white solid precipitated. The solid was filtered and washed with DCM. The aqueous layer of the combined solution was separated and washed twice with DCM. The combined organic solution was dried over Na₂SO₄ and concentrated. The crude product was purified by ISCO silica gel column (40 g) using 0-100% EtOAc/Hexanes. Isolated ethyl 4-hydroxy-4-methylpiperidine-1-carboxylate (8.7 g 79.5% yield). ¹H NMR (500 MHz, Chloroform-d) δ 4.13 (q, J=7.1 Hz, 2H), 3.78 (s, 2H), 3.28 (dt, J=14.2, 7.6 Hz, 2H), 1.56 (d, J=5.3 Hz, 4H), 1.29-1.22 (m, 6H).

### Step 2: ethyl 4-(4-bromophenyl)-4-methylpiperidine-1-carboxylate

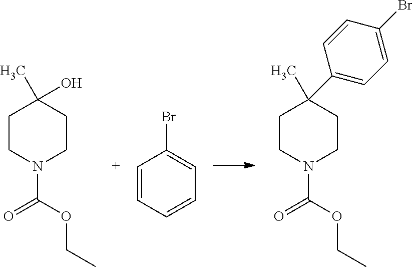 [SMILES: CCOC(=O)N1CCC(C)(O)CC1 ; Brc1ccccc1 ; CCOC(=O)N1CCC(C)(c2ccc(Br)cc2)CC1]

Ethyl 4-hydroxy-4-methylpiperidine-1-carboxylate (3.08 g, 16.45 mmol) in bromobenzene (25.83 g, 164.50 mmol), cooled to 0° C., trifluoromethanesulfonic acid (24.69 g, 164.50 mmol) was added. The resulting mixture was stirred at r.t. for 3 hrs. The solution was poured into ice, basified with 1N NaOH solution and extracted with DCM three times. The combined organic layers were washed with brine, dried over Na₂SO₄, concentrated. The crude oil was purified by ISCO silica gel column (40 g) using EtOAc/hexane (0-50%), obtained ethyl 4-(4-bromophenyl)-4-methylpiperidine-1-carboxylate (4.3 g, 80.1% yield). ¹H NMR (500 MHz, Chloroform-d) δ 7.48-7.43 (m, 2H), 7.23-7.17 (m, 2H), 4.12 (q, J=7.1 Hz, 2H), 3.56-3.48 (m, 2H), 3.46-3.38 (m, 2H), 2.03 (br, 2H), 1.68 (br, 2H), 1.28-1.21 (m, 6H). LCMS: C₁₋₅H₂₀BrNO₂ requires: 325, found: m/z=326 [M+H]⁺.

### Step 3: 4-(4-bromophenyl)-4-methylpiperidine

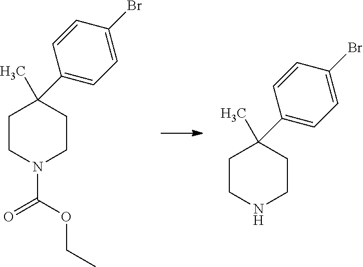 [SMILES: CCOC(=O)N1CCC(C)(c2ccc(Br)cc2)CC1 ; CC1(c2ccc(Br)cc2)CCNCC1]

To a solution of ethyl 4-(4-bromophenyl)-4-methylpiperidine-1-carboxylate (7.00 g, 21.46 mmol) in EtOH (75 mL) was added potassium hydroxide (24.08 g, 429.14 mmol), the solution was heated at 80° C. overnight. LCMS showed no starting material left. The solvent was evaporated by reduced pressure, the residue was dissolved in DCM (50 mL), washed by water (20 mL). The aqueous layer was extracted with DCM (20 mL×5), and the combined organic layers were washed with brine, dried over Na₂SO₄, and concentrated to give 5.45 g of 4-(4-bromophenyl)-4-methylpiperidine as crude product in quantitative yield, which was used directly to the next step without further purification. LCMS: C₁₂H₁₆BrN requires: 253, found: m/z=254 [M+H]⁺.

### Step 4: tert-butyl 4-(4-bromophenyl)-4-methylpiperidine-1-carboxylate

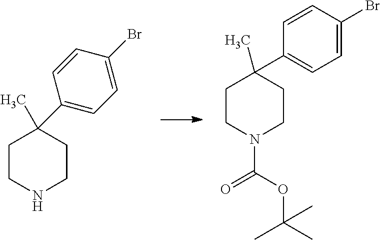 [SMILES: CC1(c2ccc(Br)cc2)CCNCC1 ; CC(C)(C)OC(=O)N1CCC(C)(c2ccc(Br)cc2)CC1]

4-(4-bromophenyl)-4-methylpiperidine (5.40 g, 21.25 mmol) was dissolved in dichloromethane (75.00 mL), di-tert-butyl dicarbonate (7.42 g, 33.99 mmol) was added slowly, and the reaction was stirred at r.t. for 1 h. The reaction solution was washed with water followed by brine, dried over Na₂SO₄, and concentrated. ISCO silica gel column purification, obtained tert-butyl 4-(4-bromophenyl)-4-methylpiperidine-1-carboxylate (7.4 g, 98.3% yield). ¹H NMR (500 MHz, Chloroform-d) δ 7.48-7.42 (m, 2H), 7.27-7.16 (m, 2H), 3.47 (ddd, J=11.8, 7.8, 3.6 Hz, 2H), 3.41-3.33 (m, 2H), 2.00 (br, 2H), 1.71-1.62 (m, 2H), 1.45 (s, 9H), 1.23 (s, 3H) LCMS: C₁₂H₁₆BrN requires: 253, found: m/z=254 [M+H]⁺.

### Step 5: tert-butyl 4-(4-aminophenyl)-4-methylpiperidine-1-carboxylate

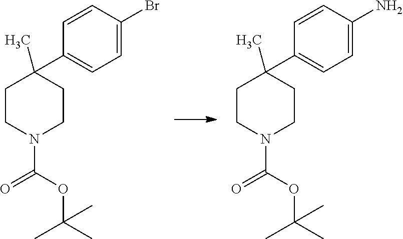 [SMILES: CC(C)(C)OC(=O)N1CCC(C)(c2ccc(Br)cc2)CC1 ; CC(C)(C)OC(=O)N1CCC(C)(c2ccc(N)cc2)CC1]

Tert-butyl 4-(4-bromophenyl)-4-methylpiperidine-1-carboxylate (2.60 g, 7.34 mmol), {[1,1′-biphenyl]-2-yl}dicyclohexylphosphane (65.00 mg, 0.19 mmol), Pd₂(dba)₃ (68.00 mg, 0.07 mmol) and LiHMDS (14.70 mL, 2.46 g, 14.68 mmol) in 15 mL anhydrous THF, the solution was bubbled with nitrogen gas and stirred at 65° C. overnight under N₂ protection. TLC showed no starting material left. The reaction mixture was diluted with DCM, washed by water and brine, dried over Na₂SO₄, concentrated. The crude product was purified by ISCO silica gel column using 0-60% EtOAc/hexane, obtained tert-butyl 4-(4-aminophenyl)-4-methylpiperidine-1-carboxylate (1.42 g, 66.6% yield). ¹H NMR (500 MHz, Chloroform-d) δ 7.14-7.08 (m, 2H), 6.70-6.64 (m, 2H), 3.58 (s, 2H), 3.49-3.44 (m, 2H), 3.39-3.31 (m, 2H), 2.00 (br, 2H), 1.64-1.58 (m, 2H) 1.45 (s, 9H), 1.20 (s, 3H) LCMS: C₁₇H₂₆N₂O₂ requires: 290, found: m/z=291 [M+H]⁺.

### Step 6: tert-butyl 4-[4-({3-cyano-6-[(3R)-3-(3-methyl-2-oxoimidazolidin-1-yl)piperidin-1-yl]pyrazin-2-yl}amino)phenyl]-4-methylpiperidine-1-carboxylate

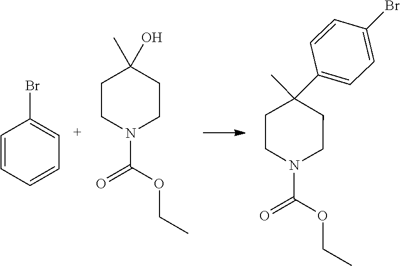

Tert-butyl 4-(4-aminophenyl)-4-methylpiperidine-1-carboxylate (0.64 g, 2.19 mmol), 3-chloro-5-[(3R)-3-(3-methyl-2-oxoimidazolidin-1-yl)piperidin-1-yl]pyrazine-2-carbonitrile (0.61 g, 1.90 mmol), cesium carbonate (1.86 g, 5.70 mmol), palladium acetate (140.89 mg, 0.63 mmol), and [2′-(diphenylphosphanyl)-[1,1′-binaphthalen]-2-yl]diphenylphosphane BINAP (390.76 mg, 0.63 mmol) in 30 mL dioxane, the solution was bubbled with nitrogen gas and heated at 115° C. for 2 hours under nitrogen protection. The reaction mixture was cooled to r.t., diluted with 250 mL EtOAc and filtered. The filtrate was concentrated and purified by ISCO silica gel column using EtOAc/DCM (0-100%), obtained tert-butyl 4-[4-({3-cyano-6-[(3R)-3-(3-methyl-2-oxoimidazolidin-1-yl)piperidin-1-yl]pyrazin-2-yl}amino)phenyl]-4-methylpiperidine-1-carboxylate (1.09 g, 100% yield). LCMS: C₃₁H₄₂N₈O₃ requires: 574, found: m/z=575 [M+H]⁺.

### Step 7: tert-butyl 4-[4-({3-carbamoyl-6-[(3R)-3-(3-methyl-2-oxoimidazolidin-1-yl)piperidin-1-yl]pyrazin-2-yl}amino)phenyl]-4-methylpiperidine-1-carboxylate

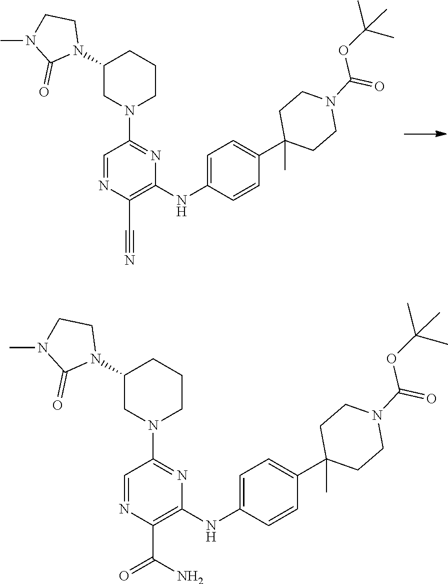

Tert-butyl 4-[4-({3-cyano-6-[(3R)-3-(3-methyl-2-oxoimidazolidin-1-yl)piperidin-1-yl]pyrazin-2-yl}amino)phenyl]-4-methylpiperidine-1-carboxylate (1.09 g, 1.9 mmol) was dissolved in methanol (25.00 mL) and DMSO (5.00 mL), cesium carbonate (325 mg, 1.0 mmol) was added, and then 30% H₂O₂ solution (2.31 g, 3 mL, 20.36 mmol) was added. Stirred at room temperature for 30 min. LCMS showed no starting material left. 10 mL acetonitrile was added, stirred for 5 min, evaporated all the solvent. The residue was dissolved in 200 mL EtOAc, washed with water three times, dried over Na₂SO₄, concentrated. ISCO silica gel column (24 g) purification using 30-100% EtOAc/Hexane, followed by 0-10% MeOH/DCM. Obtained tert-butyl 4-[4-({3-carbamoyl-6-[(3R)-3-(3-methyl-2-oxoimidazolidin-1-yl)piperidin-1-yl]pyrazin-2-yl}amino)phenyl]-4-methylpiperidine-1-carboxylate (1.00 g, 82.9% yield). ¹H NMR (500 MHz, Chloroform-d) δ 10.84 (s, 1H), 7.60 (d, J=8.8 Hz, 2H), 7.49 (s, 1H), 7.26 (d, J=8.8 Hz, 2H), 5.18 (s, 1H), 4.36 (t, J=11.6 Hz, 2H), 3.81 (m, 1H), 3.49 (br, 2H), 3.43-3.26 (m, 5H), 3.08 (t, J=11.7 Hz, 1H), 2.98-2.92 (m, 1H), 2.82 (s, 3H), 2.05 (d, J=9.1 Hz, 2H), 2.02-1.97 (m, 1H), 1.90 (dt, J=13.3, 3.3 Hz, 1H), 1.76 (td, J=11.7, 3.5 Hz, 1H), 1.82-1.65 (m, 3H), 1.45 (s, 9H), 1.25 (s, 3H). LCMS: C₃₁H₄₄N₈O₄ requires: 592, found: m/z=593 [M+H]⁺.

### Step 8: 5-[(3R)-3-(3-methyl-2-oxoimidazolidin-1-yl)piperidin-1-yl]-3-{[4-(4-methylpiperidin-4-yl)phenyl]amino}pyrazine-2-carboxamide

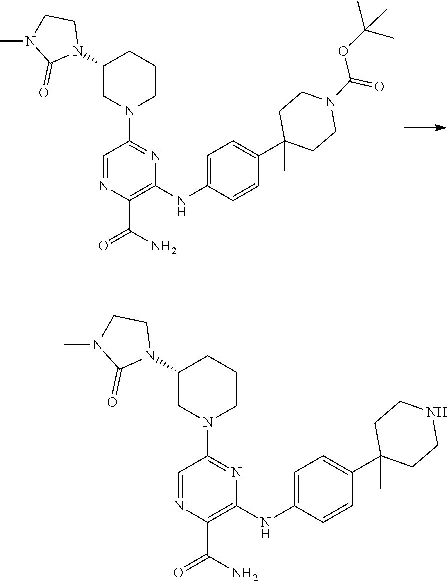

Tert-butyl 4-[4-({3-carbamoyl-6-[(3R)-3-(3-methyl-2-oxoimidazolidin-1-yl)piperidin-1-yl]pyrazin-2-yl}amino)phenyl]-4-methylpiperidine-1-carboxylate (200.00 mg, 0.34 mmol) was dissolved in 4N HCl in dioxane (2 mL), stirred at r.t. for 30 min, evaporated solvent to give 5-[(3R)-3-(3-methyl-2-oxoimidazolidin-1-yl)piperidin-1-yl]-3-{[4-(4-methylpiperidin-4-yl)phenyl]amino}pyrazine-2-carboxamide as crude product in quantitative yield, which was used directly to the next step without further purification. LCMS: C₂₆H₃₆N₈O₂ requires: 492, found: m/z=493 [M+H]⁺.

### Example 3: Synthesis of 3-{[4-(azetidin-3-yl)phenyl]amino}-5-[(3R)-3-(3-methyl-2-oxoimidazolidin-1-yl)piperidin-1-yl]pyrazine-2-carboxamide

### Step 1: tert-butyl 3-[4-({3-cyano-6-[(3R)-3-(3-methyl-2-oxoimidazolidin-1-yl)piperidin-1-yl]pyrazin-2-yl}amino)phenyl]azetidine-1-carboxylate

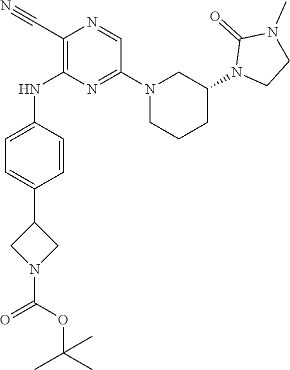 [SMILES: CN1CCN([C@@H]2CCCN(c3cnc(C#N)c(Nc4ccc(C5CN(C(=O)OC(C)(C)C)C5)cc4)n3)C2)C1=O]

3-chloro-5-[(3R)-3-(3-methyl-2-oxoimidazolidin-1-yl)piperidin-1-yl]pyrazine-2-carbonitrile (207 mg, 0.65 mmol), tert-butyl 3-(4-aminophenyl)azetidine-1-carboxylate (160 mg, 0.65 mmol), and cesium carbonate (847 mg, 2.60 mmol) were deposited in a vial with dioxane (5 mL). A vacuum was pulled on the vial until the mixture bubbled then the headspace was backfilled with argon for 5 cycles. BINAP (80.4 mg, 0.13 mmol) and palladium (II) acetate (29.0 mg, 0.13 mmol) were added. A vacuum was pulled on the vial and the headspace was backfilled with argon for 5 cycles. The mixture was heated at 90° C. overnight. The mixture was diluted with water and extracted twice with DCM. The combined organic layers were dried over anhydrous Na₂SO₄ and concentrated in vacuo. The crude residue was purified by flash chromatography on a 24 g silica gel column eluted with 0 to 10% MeOH/ethyl acetate gradient to provide tert-butyl 3-[4-({3-cyano-6-[(3R)-3-(3-methyl-2-oxoimidazolidin-1-yl)piperidin-1-yl]pyrazin-2-yl}amino)phenyl]azetidine-1-carboxylate (257 mg, 74.8%). LCMS: C₂₈H₃₆N₈O₃ requires 532, found: m/z=533 [M+H]⁺.

### Step 2: tert-butyl 3-[4-({3-carbamoyl-6-[(3R)-3-(3-methyl-2-oxoimidazolidin-1-yl)piperidin-1-yl]pyrazin-2-yl}amino)phenyl]azetidine-1-carboxylate

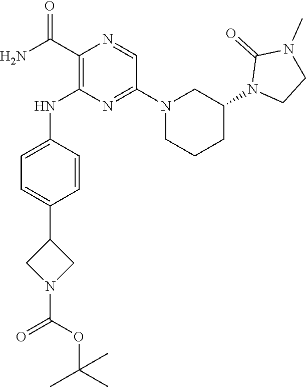 [SMILES: CN1CCN([C@@H]2CCCN(c3cnc(C(N)=O)c(Nc4ccc(C5CN(C(=O)OC(C)(C)C)C5)cc4)n3)C2)C1=O]

Tert-butyl 3-[4-({3-cyano-6-[(3R)-3-(3-methyl-2-oxoimidazolidin-1-yl)piperidin-1-yl]pyrazin-2-yl}amino)phenyl]azetidine-1-carboxylate (257 mg, 0.48 mmol) was dissolved in MeOH (6 mL) and DMSO (3 mL). Cesium carbonate (157 mg, 0.48 mmol) was added followed by 1.5 mL 35% H₂O₂. After 3 hours, 4 mL ACN was added. After 20 minutes, the mixture was diluted with ethyl acetate and washed 3× with water. The organic layer was dried over Na₂SO₄ and concentrated in vacuo. The crude residue was purified by flash chromatography on a 24 g silica gel column eluted with 0 to 10% MeOH/ethyl acetate gradient to provide tert-butyl 3-[4-({3-carbamoyl-6-[(3R)-3-(3-methyl-2-oxoimidazolidin-1-yl)piperidin-1-yl]pyrazin-2-yl}amino)phenyl]azetidine-1-carboxylate (261 mg, 98.2%). LCMS: C₂₈H₃₈N₈O₄ requires 550, found m/z=551 [M+H]⁺.

### Step 3: 3-{[4-(azetidin-3-yl)phenyl]amino}-5-[(3R)-3-(3-methyl-2-oxoimidazolidin-1-yl)piperidin-1-yl]pyrazine-2-carboxamide

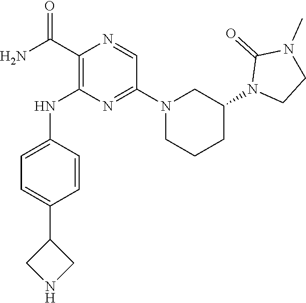 [SMILES: CN1CCN([C@@H]2CCCN(c3cnc(C(N)=O)c(Nc4ccc(C5CNC5)cc4)n3)C2)C1=O]

Tert-butyl 3-[4-({3-carbamoyl-6-[(3R)-3-(3-methyl-2-oxoimidazolidin-1-yl)piperidin-1-yl]pyrazin-2-yl}amino)phenyl]azetidine-1-carboxylate (261.00 mg, 0.47 mmol) was stirred in DCM (1 mL) and TFA (1 mL) for 15 minutes and was concentrated in vacuo then lyophilized to provide 3-{[4-(azetidin-3-yl)phenyl]amino}-5-[(3R)-3-(3-methyl-2-oxoimidazolidin-1-yl)piperidin-1-yl]pyrazine-2-carboxamide (265 mg, 100%). LCMS: C₂₃H₃₀N₈O₂ requires 451, found: m/z=451 [M+H]⁺.

### Example 4: Synthesis of (R)-3-((4-(2,6-diazaspiro[3.3]heptan-2-yl)phenyl)amino)-5-(3-(3-methyl-2-oxoimidazolidin-1-yl)piperidin-1-yl)pyrazine-2-carboxamide

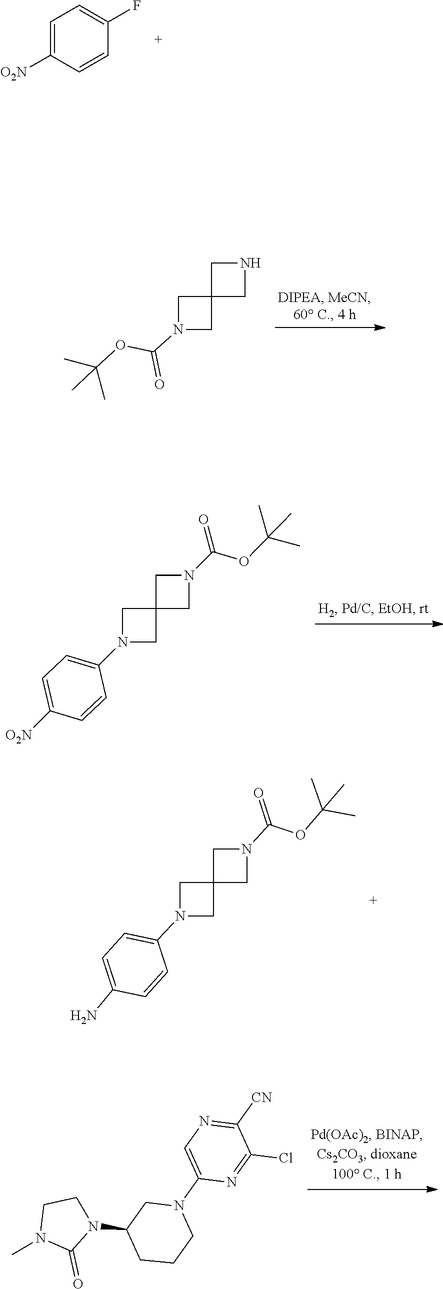

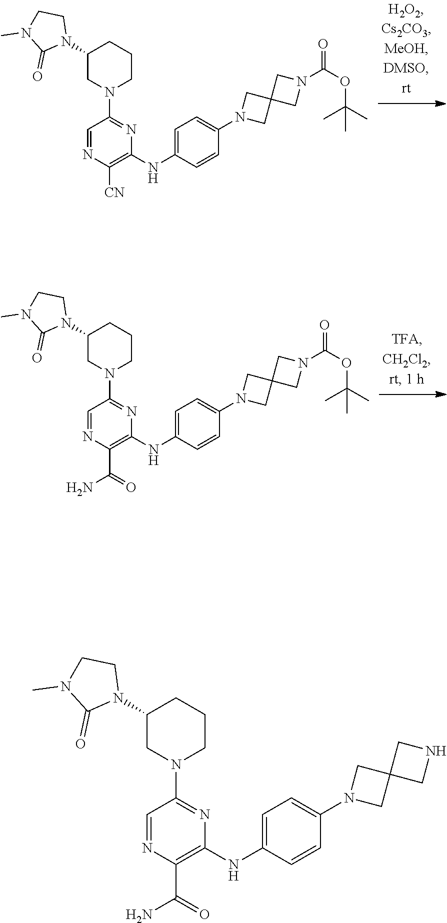

### Step 1: tert-butyl 6-(4-nitrophenyl)-2,6-diazaspiro[3.3]heptane-2-carboxylate

A mixture of tert-butyl 2,6-diazaspiro[3.3]heptane-2-carboxylate (515 mg, 2.60 mmol), MeCN (2 mL), ethylbis(propan-2-yl)amine (1.81 mL, 10.4 mmol) and 4-fluoronitrobenzene (367 mg, 2.60 mmol) was allowed to stir at 60° C. for 4 h. EtOAc and H₂O were added. The organic layer was dried with MgSO₄, filtered, concentrated and purified by MPLC (0-50% EtOAc in hexanes) to afford tert-butyl 6-(4-nitrophenyl)-2,6-diazaspiro[3.3]heptane-2-carboxylate (491 mg, 59.2%). LCMS: C₁₆H₂₁N₃O₄ requires 319, found: m/z=320 [M+H]⁺.

### Step 2: tert-butyl 6-(4-aminophenyl)-2,6-diazaspiro[3.3]heptane-2-carboxylate

A mixture of Pd/C (16 mg, 0.15 mmol), EtOH (15 mL), tert-butyl 6-(4-nitrophenyl)-2,6-diazaspiro[3.3]heptane-2-carboxylate (491 mg, 1.54 mmol) was evacuated and backfilled with H₂ 5 times. The mixture was allowed to stir at r.t. for 2 h. The mixture was filtered through celite washing with EtOAc/MeOH, concentrated to afford tert-butyl 6-(4-aminophenyl)-2,6-diazaspiro[3.3]heptane-2-carboxylate (439 mg, 98.7%). LCMS: C₁₆H₂₃N₃O₂ requires 289, found: m/z=290 [M+H]⁺.

### Step 3: tert-butyl (R)-6-(4-((3-cyano-6-(3-(3-methyl-2-oxoimidazolidin-1-yl)piperidin-1-yl)pyrazin-2-yl)amino)phenyl)-2,6-diazaspiro[3.3]heptane-2-carboxylate

A mixture of tert-butyl 6-(4-aminophenyl)-2,6-diazaspiro[3.3]heptane-2-carboxylate (245 mg, 0.85 mmol),3-chloro-5-[(3R)-3-(3-methyl-2-oxoimidazolidin-1-yl)piperidin-1-yl]pyrazine-2-carbonitrile (272 mg, 0.85 mmol), Pd(OAc)₂ (62.8 mg, 0.28 mmol), [2′-(diphenylphosphanyl)-[1,1′-binaphthalen]-2-yl]diphenylphosphane (174 mg, 0.28 mmol) and cesium carbonate (829 mg, 2.54 mmol) was degassed and backfilled with N₂ 5 times. Dioxane (4 mL) was added. The mixture was allowed to stir at 100° C. for 90 min. The mixture was filtered through celite washing with MeOH/EtOAc, concentrated and purified by MPLC (0-100% EtOAc in CH₂C₁₂) to afford tert-butyl 6-[4-({3-cyano-6-[(3R)-3-(3-methyl-2-oxoimidazolidin-1-yl)piperidin-1-yl]pyrazin-2-yl}amino)phenyl]-2,6-diazaspiro[3.3]heptane-2-carboxylate (272 mg, 55.9%). LCMS: C₃₀H₃₉N₉O₃ requires 573, found: m/z=574 [M+H]⁺.

### Step 4: tert-butyl (R)-6-(4-((3-carbamoyl-6-(3-(3-methyl-2-oxoimidazolidin-1-yl)piperidin-1-yl)pyrazin-2-yl)amino)phenyl)-2,6-diazaspiro[3.3]heptane-2-carboxylate

H₂O₂(30% in H₂O, 0.80 mL, 0.08 mmol) was added to a mixture of tert-butyl 6-[4-({3-cyano-6-[(3R)-3-(3-methyl-2-oxoimidazolidin-1-yl)piperidin-1-yl]pyrazin-2-yl}amino)phenyl]-2,6-diazaspiro[3.3]heptane-2-carboxylate (272 mg, 0.47 mmol), cesium carbonate (154 mg, 0.47 mmol), MeOH (10 mL) and DMSO (0.5 mL). The mixture was allowed to stir at r.t. for 30 min. The mixture was concentrated. EtOAc was added and the organic phase was washed with H₂O and brine. The organic layer was dried with MgSO₄, filtered, concentrated and purified by MPLC (0-10% MeOH in CH₂Cl₂) to afford tert-butyl 6-[4-({3-carbamoyl-6-[(3R)-3-(3-methyl-2-oxoimidazolidin-1-yl)piperidin-1-yl]pyrazin-2-yl}amino)phenyl]-2,6-diazaspiro[3.3]heptane-2-carboxylate (142 mg, 50.6%). LCMS: C₃₀H₄₁N₉O₄ requires 591, found: m/z=592 [M+H]⁺.

### Step 5: (R)-3-((4-(2,6-diazaspiro[3.3]heptan-2-yl)phenyl)amino)-5-(3-(3-methyl-2-oxoimidazolidin-1-yl)piperidin-1-yl)pyrazine-2-carboxamide

A mixture of tert-butyl 6-[4-({3-carbamoyl-6-[(3R)-3-(3-methyl-2-oxoimidazolidin-1-yl)piperidin-1-yl]pyrazin-2-yl}amino)phenyl]-2,6-diazaspiro[3.3]heptane-2-carboxylate (142 mg, 0.24 mmol), CH₂C₁₂ (2 mL) and TFA (0.4 mL) was allowed to stir at r.t. for 1 h. The volatiles were removed to afford 3-[(4-{2,6-diazaspiro[3.3]heptan-2-yl}phenyl)amino]-5-[(3R)-3-(3-methyl-2-oxoimidazolidin-1-yl)piperidin-1-yl]pyrazine-2-carboxamide (117 mg, 99.2%). LCMS: C₂₅H₃₃N₉O₂ requires 491, found: m/z=492 [M+H]⁺.

### Example 5: Synthesis of (R)-3-((4-(3,9-diazaspiro[5.5]undecan-3-yl)phenyl)amino)-5-(3-(3-methyl-2-oxoimidazolidin-1-yl)piperidin-1-yl)pyrazine-2-carboxamide

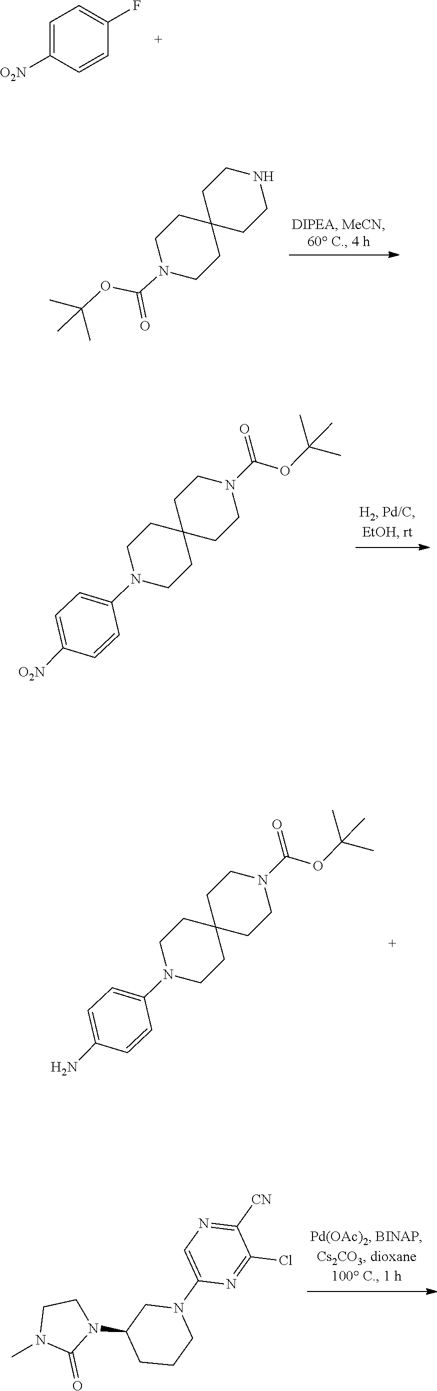

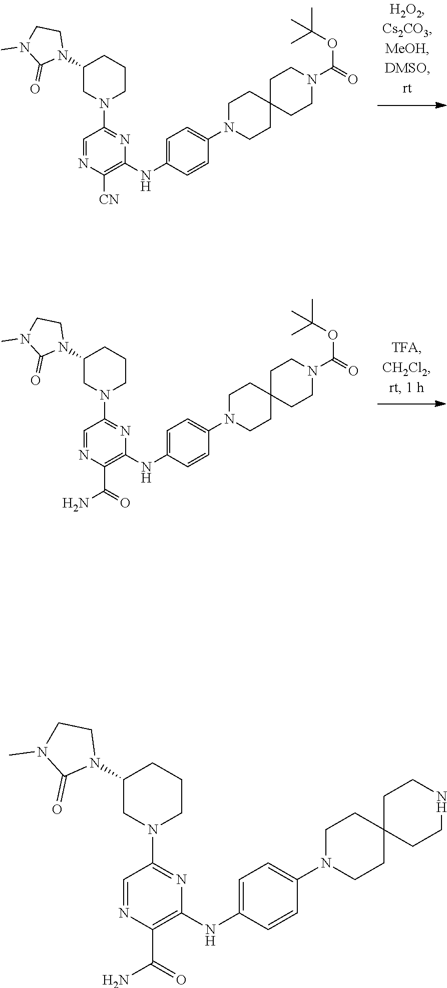

### Step 1: tert-butyl 9-(4-nitrophenyl)-3,9-diazaspiro[5.5]undecane-3-carboxylate

A mixture of 4-fluoronitrobenzene (554.7 mg, 3.93 mmol), DMF (20 mL), ethylbis(propan-2-yl)amine (2.74 mL, 15.7 mmol) and tert-butyl 3,9-diazaspiro[5.5]undecane-3-carboxylate (1000 mg, 3.93 mmol) was allowed to stir at 90° C. overnight. EtOAc and H₂O were added. The organic layer was dried with MgSO₄, filtered, concentrated and purified by MPLC (0-50% EtOAc in hexanes) to afford tert-butyl 9-(4-nitrophenyl)-3,9-diazaspiro[5.5]undecane-3-carboxylate (1287.00 mg, 87.2%). C₂₀H₂₉N₃O₄ requires 375, found: m/z=376 [M+H]⁺.

### Step 2: tert-butyl 9-(4-aminophenyl)-3,9-diazaspiro[5.5]undecane-3-carboxylate

A mixture of tert-butyl 9-(4-nitrophenyl)-3,9-diazaspiro[5.5]undecane-3-carboxylate (1.29 g, 3.43 mmol), Pd/C (36 mg, 0.34 mmol), EtOH (30 mL) was evacuated and backfilled with H₂ 5 times. The mixture was allowed to stir at r.t. for 2 h. The mixture was filtered through celite washing with EtOAc/MeOH, concentrated to afford tert-butyl 9-(4-aminophenyl)-3,9-diazaspiro[5.5]undecane-3-carboxylate (871 mg, 73.5%). LCMS: C₂₀H₃₁N₃O₂ requires 345, found: m/z=346 [M+H]⁺.

### Step 3: tert-butyl (R)-9-(4-((3-cyano-6-(3-(3-methyl-2-oxoimidazolidin-1-yl)piperidin-1-yl)pyrazin-2-yl)amino)phenyl)-3,9-diazaspiro[5.5]undecane-3-carboxylate

A mixture of tert-butyl 9-(4-aminophenyl)-3,9-diazaspiro[5.5]undecane-3-carboxylate (162.6 mg, 0.47 mmol), 3-chloro-5-[(3R)-3-(3-methyl-2-oxoimidazolidin-1-yl)piperidin-1-yl]pyrazine-2-carbonitrile (151 mg, 0.47 mmol), Pd(OAc)₂ (34.9 mg, 0.16 mmol), [2′-(diphenylphosphanyl)-[1,1′-binaphthalen]-2-yl]diphenylphosphane (96.7 mg, 0.16 mmol) and cesium carbonate (460 mg, 1.41 mmol) was degassed and backfilled with N₂ 5 times. The mixture was allowed to stir at 100° C. for 90 min. The mixture was filtered through celite washing with MeOH/EtOAc, concentrated and purified by MPLC (0-100% EtOAc in CH₂C₁₂) to afford tert-butyl 9-[4-({3-cyano-6-[(3R)-3-(3-methyl-2-oxoimidazolidin-1-yl)piperidin-1-yl]pyrazin-2-yl}amino)phenyl]-3,9-diazaspiro[5.5]undecane-3-carboxylate (204 mg, 68.8%). LCMS: C₃₄H₄₇N₉O₃ requires 629, found: m/z=630 [M+H]⁺.

### Step 4: tert-butyl (R)-9-(4-((3-carbamoyl-6-(3-(3-methyl-2-oxoimidazolidin-1-yl)piperidin-1-yl)pyrazin-2-yl)amino)phenyl)-3,9-diazaspiro[5.5]undecane-3-carboxylate

H₂O₂(30% in H₂O, 0.55 mL, 0.00 g, 0.05 mmol) was added to a mixture of tert-butyl 9-[4-({3-cyano-6-[(3R)-3-(3-methyl-2-oxoimidazolidin-1-yl)piperidin-1-yl]pyrazin-2-yl}amino)phenyl]-3,9-diazaspiro[5.5]undecane-3-carboxylate (204 mg, 0.32 mmol), cesium carbonate (106 mg, 0.32 mmol), MeOH (6 mL) and DMSO (0.3 mL). The mixture was allowed to stir at r.t. for 30 min. The mixture was concentrated. EtOAc was added and the organic phase was washed with H₂O and brine. The organic layer was dried with MgSO₄, filtered, concentrated and purified by MPLC (0-10% MeOH in CH₂Cl₂) to afford tert-butyl 9-[4-({3-carbamoyl-6-[(3R)-3-(3-methyl-2-oxoimidazolidin-1-yl)piperidin-1-yl]pyrazin-2-yl}amino)phenyl]-3,9-diazaspiro[5.5]undecane-3-carboxylate (95.00 mg, 45%). LCMS: C₃₄H₄₉N₉O₄ requires 647, found: m/z=648 [M+H]⁺.

### Step 5: (R)-3-((4-(3,9-diazaspiro[5.5]undecan-3-yl)phenyl)amino)-5-(3-(3-methyl-2-oxoimidazolidin-1-yl)piperidin-1-yl)pyrazine-2-carboxamide

A mixture of tert-butyl 9-[4-({3-carbamoyl-6-[(3R)-3-(3-methyl-2-oxoimidazolidin-1-yl)piperidin-1-yl]pyrazin-2-yl}amino)phenyl]-3,9-diazaspiro[5.5]undecane-3-carboxylate (25 mg, 0.04 mmol), CH₂C₁₂ (1 mL) and TFA (0.2 mL) was allowed to stir at r.t. for 1 h. The volatiles were removed to afford 3-[(4-{3,9-diazaspiro[5.5]undecan-3-yl}phenyl)amino]-5-[(3R)-3-(3-methyl-2-oxoimidazolidin-1-yl)piperidin-1-yl]pyrazine-2-carboxamide (21.00 mg, 99.4%). LCMS: C₂₉H₄₁N₉O₂ requires 547, found: m/z=548 [M+H]⁺.

### Example 6: Synthesis of (R)-3-((4-(2,9-diazaspiro[5.5]undecan-9-yl)phenyl)amino)-5-(3-(3-methyl-2-oxoimidazolidin-1-yl)piperidin-1-yl)pyrazine-2-carboxamide

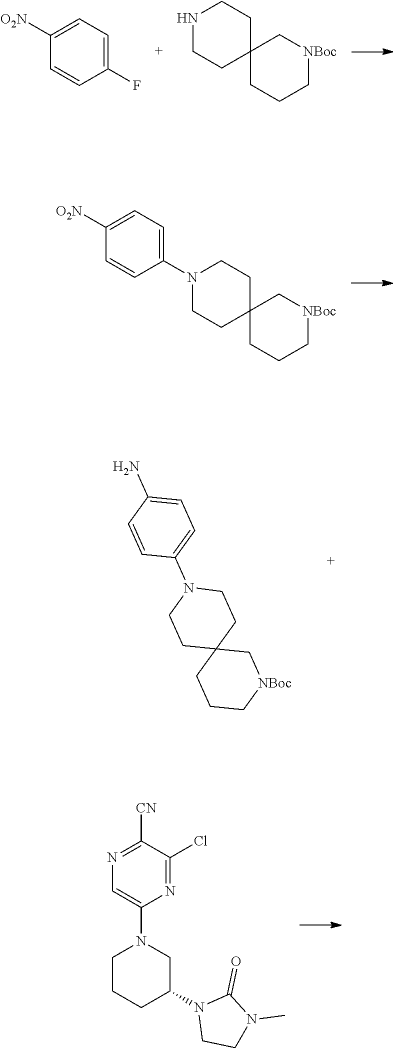 [SMILES: O=[N+]([O-])c1ccc(F)cc1 ; CC(C)(C)OC(=O)N1CCCC2(CCNCC2)C1 ; CC(C)(C)OC(=O)N1CCCC2(CCN(c3ccc([N+](=O)[O-])cc3)CC2)C1 ; CC(C)(C)OC(=O)N1CCCC2(CCN(c3ccc(N)cc3)CC2)C1 ; CN1CCN([C@@H]2CCCN(c3cnc(C#N)c(Cl)n3)C2)C1=O]

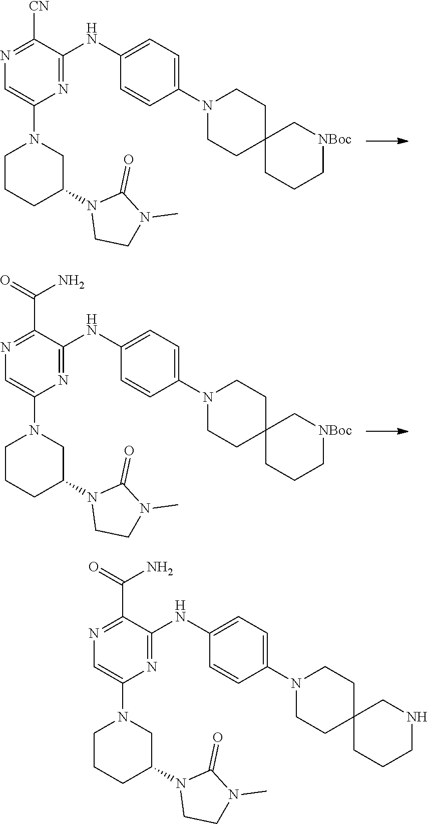

### Step 1: tert-butyl 9-(4-nitrophenyl)-2,9-diazaspiro[5.5]undecane-2-carboxylate

Para-fluoronitrobenzene (1 eq) and spirocyclic amine (1 eq) were combined in DMF, followed by addition of potassium carbonate (2 eq). The reaction mixture was stirred at 65° C. for 5 h, then cooled to room temperature. The reaction mixture was then partitioned between ethyl acetate and water, and the organic layer was separated, dried over magnesium sulfate, and filtered. This solution was concentrated to afford tert-butyl 9-(4-nitrophenyl)-2,9-diazaspiro[5.5]undecane-2-carboxylate. LCMS C₂₀H₂₉N₃O₄ requires: 375.5, found: m/z=376.6 [M+H]⁺.

### Step 2: tert-butyl 9-(4-aminophenyl)-2,9-diazaspiro[5.5]undecane-2-carboxylate

The crude material from step 1 of this Example 6 was dissolved in ethanol and water (10:1). Ammonium chloride (3.5 eq) and iron (3 eq) were added, followed by vigorous stirring and heating to 90° C. for 4 h. The reaction was then filtered with Celite while still hot, and the Celite was further washed with ethyl acetate. The resulting solution was partitioned between ethyl acetate and water. The water layer was separated and re-extracted with ethyl acetate. The combined organic layers were washed with brine, dried over magnesium sulfate, and concentrated. Silica gel chromatography provided tert-butyl 9-(4-aminophenyl)-2,9-diazaspiro[5.5]undecane-2-carboxylate (52% over 2 steps). LCMS C₂₀H₃₁N₃O₂ requires: 345.59, found: m/z=346.5 [M+H]⁺.

### Step 3: tert-butyl (R)-9-(4-((3-cyano-6-(3-(3-methyl-2-oxoimidazolidin-1-yl)piperidin-1-yl)pyrazin-2-yl)amino)phenyl)-2,9-diazaspiro[5.5]undecane-2-carboxylate

Chloropyrimidine intermediate (R)-3-chloro-5-(3-(3-methyl-2-oxoimidazolidin-1-yl)piperidin-1-yl)pyrazine-2-carbonitrile, tert-butyl 9-(4-aminophenyl)-2,9-diazaspiro[5.5]undecane-2-carboxylate, Pd(OAc)₂ (0.15 eq), BINAP (0.15 eq), and cesium carbonate (2 eq) were combined in a microwave tube, followed by addition of dioxane (0.25 M). Nitrogen was bubbled through for 30 seconds, followed by capping. Heating to 9 0° C., followed by maintaining that temperature for 3 h provided a dark reaction mixture which was monitored by LCMS. The reaction was then cooled, and filtered through Celite, washing with ethyl acetate/methanol. The crude material was loaded onto silica and chromatographed (silica, 0-10% methanol in DCM), to provide tert-butyl (R)-9-(4-((3-cyano-6-(3-(3-methyl-2-oxoimidazolidin-1-yl)piperidin-1-yl)pyrazin-2-yl)amino)phenyl)-2,9-diazaspiro[5.5]undecane-2-carboxylate (40%). LCMS C₃₄H₄₇N₉O₃ requires: 629.81, found: m/z=630.7 [M+H]⁺.

### Step 4: tert-butyl (R)-9-(4-((3-carbamoyl-6-(3-(3-methyl-2-oxoimidazolidin-1-yl)piperidin-1-yl)pyrazin-2-yl)amino)phenyl)-2,9-diazaspiro[5.5]undecane-2-carboxylate

This material was then dissolved in methanol/DMSO (10:1) and a pellet of NaOH was added. The reaction was stirred for 5 minutes, followed by addition of 35% peroxide solution (2 mL of solution per mmol of reactant). This reaction mixture was stirred for 3 h, then partitioned between ethyl acetate and water. The organic layer was separated and dried over magnesium sulfate. Chromatography (0-10% methanol in DCM) provided (R)-3-((4-(2,9-diazaspiro[5.5]undecan-9-yl)phenyl)amino)-5-(3-(3-methyl-2-oxoimidazolidin-1-yl)piperidin-1-yl)pyrazine-2-carboxamide (90% yield). LCMS C₃₄H₄₉N₉O₄ requires: 647.8, found: m/z=648.7[M+H]⁺.

### Step 5: (R)-3-((4-(2,9-diazaspiro[5.5]undecan-9-yl)phenyl)amino)-5-(3-(3-methyl-2-oxoimidazolidin-1-yl)piperidin-1-yl)pyrazine-2-carboxamide

(R)-3-((4-(2,9-diazaspiro[5.5]undecan-9-yl)phenyl)amino)-5-(3-(3-methyl-2-oxoimidazolidin-1-yl)piperidin-1-yl)pyrazine-2-carboxamide was dissolved in DCM:TFA (5:1 ratio, 0.2M) and the reaction was stirred for 4 h. The reaction mixture was concentrated by rotary evaporator, followed by suspension in diethyl ether. This suspension was sonicated, followed by concentration by rotary evaporator and further drying for 16 h to afford (R)-3-((4-(2,9-diazaspiro[5.5]undecan-9-yl)phenyl)amino)-5-(3-(3-methyl-2-oxoimidazolidin-1-yl)piperidin-1-yl)pyrazine-2-carboxamide. LCMS C₂₉H₄₁N₉O₂ requires: 547.7, found: m/z=548.6 [M+H]⁺.

### Example 7: Synthesis of (R)-5-(3-(3-methyl-2-oxoimidazolidin-1-yl)piperidin-1-yl)-3-((1,2,3,4-tetrahydroisoquinolin-6-yl)amino)pyrazine-2-carboxamide

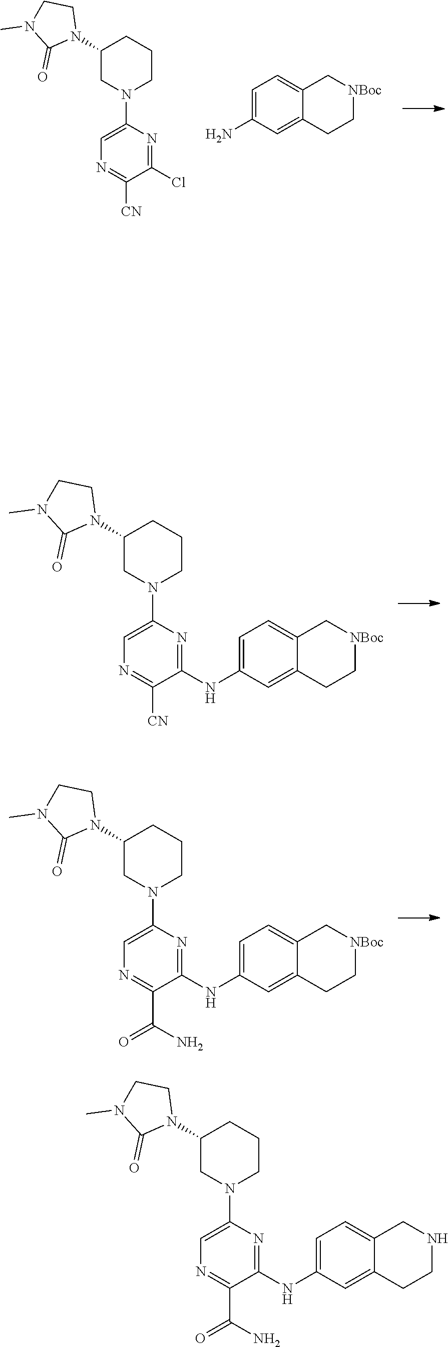

### Step 1: tert-butyl (R)-6-((3-cyano-6-(3-(3-methyl-2-oxoimidazolidin-1-yl)piperidin-1-yl)pyrazin-2-yl)amino)-3,4-dihydroisoquinoline-2(1H)-carboxylate

(R)-3-chloro-5-(3-(3-methyl-2-oxoimidazolidin-1-yl)piperidin-1-yl)pyrazine-2-carbonitrile, aniline, Pd(OAc)₂ (0.15 eq), BINAP (0.15 eq), and cesium carbonate (2 eq) were combined in a microwave tube, followed by addition of dioxane (0.25 M). Nitrogen was bubbled through for 30 seconds, followed by capping. Heating to 90° C., followed by maintaining that temperature for 3 h provided a dark reaction mixture which was monitored by LCMS. The reaction was then cooled, and filtered through Celite, washing with ethyl acetate/methanol. The crude material was loaded onto silica and chromatographed (silica, 0-10% methanol in DCM), to provide tert-butyl (R)-6-((3-cyano-6-(3-(3-methyl-2-oxoimidazolidin-1-yl)piperidin-1-yl)pyrazin-2-yl)amino)-3,4-dihydroisoquinoline-2(1H)-carboxylate. LCMS C₂₈H₃₆N₈O₃ requires: 532.7, found: m/z=533.5 [M+H]⁺.

### Step 2: tert-butyl (R)-6-((3-carbamoyl-6-(3-(3-methyl-2-oxoimidazolidin-1-yl)piperidin-1-yl)pyrazin-2-yl)amino)-3,4-dihydroisoquinoline-2(1H)-carboxylate

Tert-butyl (R)-6-((3-cyano-6-(3-(3-methyl-2-oxoimidazolidin-1-yl)piperidin-1-yl)pyrazin-2-yl)amino)-3,4-dihydroisoquinoline-2(1H)-carboxylate was dissolved in methanol/DMSO (10:1) and a pellet of NaOH was added. The reaction was stirred for 5 minutes, followed by addition of 35% peroxide solution (2 mL of solution per mmol of reactant). This reaction mixture was stirred for 3 h, then partitioned between ethyl acetate and water. The organic layer was separated and dried over magnesium sulfate. Chromatography (0-10% methanol in DCM) provided tert-butyl (R)-6-((3-carbamoyl-6-(3-(3-methyl-2-oxoimidazolidin-1-yl)piperidin-1-yl)pyrazin-2-yl)amino)-3,4-dihydroisoquinoline-2(1H)-carboxylate (18% over 2 steps). LCMS C₂₈H₃₈N₈O₄ requires: 550.7, found: m/z=551.7 [M+H]⁺.

### Step 3: (R)-5-(3-(3-methyl-2-oxoimidazolidin-1-yl)piperidin-1-yl)-3-((1,2,3,4-tetrahydroisoquinolin-6-yl)amino)pyrazine-2-carboxamide

Tert-butyl (R)-6-((3-carbamoyl-6-(3-(3-methyl-2-oxoimidazolidin-1-yl)piperidin-1-yl)pyrazin-2-yl)amino)-3,4-dihydroisoquinoline-2(1H)-carboxylate was dissolved in DCM:TFA (5:1 ratio, 0.2M) and the reaction was stirred for 4 h. The reaction mixture was concentrated by rotary evaporator, followed by suspension in diethyl ether. This suspension was sonicated, followed by concentration by rotary evaporator and further drying for 16 h, then used in the next step. LCMS C₂₃H₃₀N₈O₂ requires: 450.5, found: m/z=451 [M+H]⁺.

### Example 8: Synthesis of (R)-3-((2-(azetidin-3-yl)-1,2,3,4-tetrahydroisoquinolin-6-yl)amino)-5-(3-(3-methyl-2-oxoimidazolidin-1-yl)piperidin-1-yl)pyrazine-2-carboxamide

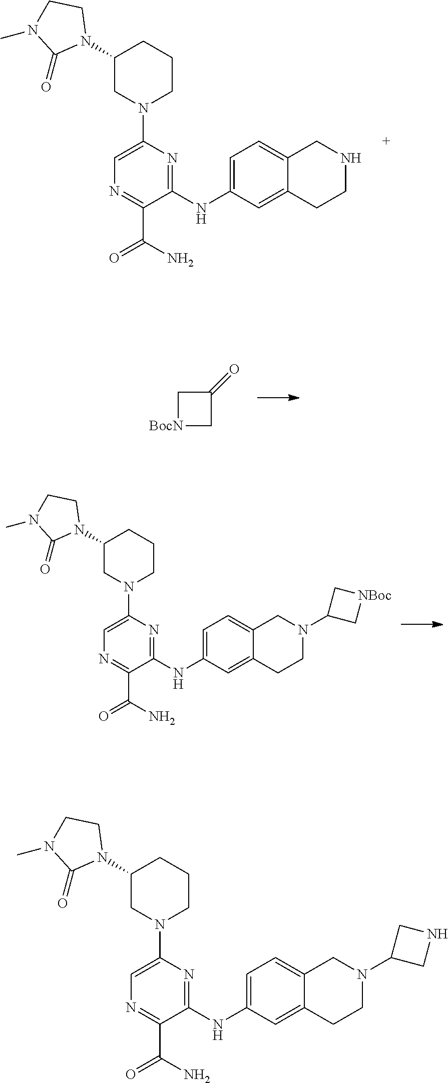 [SMILES: CN1CCN([C@@H]2CCCN(c3cnc(C(N)=O)c(Nc4ccc5c(c4)CCNC5)n3)C2)C1=O ; CC(C)(C)OC(=O)N1CC(=O)C1 ; CN1CCN([C@@H]2CCCN(c3cnc(C(N)=O)c(Nc4ccc5c(c4)CCN(C4CN(C(=O)OC(C)(C)C)C4)C5)n3)C2)C1=O ; CN1CCN([C@@H]2CCCN(c3cnc(C(N)=O)c(Nc4ccc5c(c4)CCN(C4CNC4)C5)n3)C2)C1=O]

### Step 1: tert-butyl (R)-3-(6-((3-carbamoyl-6-(3-(3-methyl-2-oxoimidazolidin-1-yl)piperidin-1-yl)pyrazin-2-yl)amino)-3,4-dihydroisoquinolin-2(1H)-yl)azetidine-1-carboxylate

(R)-5-(3-(3-methyl-2-oxoimidazolidin-1-yl)piperidin-1-yl)-3-((1,2,3,4-tetrahydroisoquinolin-6-yl)amino)pyrazine-2-carboxamide was combined with tert-butyl 3-oxoazetidine-1-carboxylate (1 equiv) and stirred in a solution of DCE and TEA (10:1, 0.1M) for 5 minutes. Sodium triacetoxyborohydride (5 equiv.) was then added, and the reaction was stirred at room temperature for 5 h. The reaction was then partitioned between ethyl acetate and water. The organic layer was separated, washed with brine, dried over magnesium sulfate, and filtered to afford tert-butyl (R)-3-(6-((3-carbamoyl-6-(3-(3-methyl-2-oxoimidazolidin-1-yl)piperidin-1-yl)pyrazin-2-yl)amino)-3,4-dihydroisoquinolin-2(1H)-yl)azetidine-1-carboxylate (90% yield). LCMS C₃₁H₄₃N₉O₄ requires: 605, found: m/z=606 [M+H]⁺.

### Step 2: (R)-3-((2-(azetidin-3-yl)-1,2,3,4-tetrahydroisoquinolin-6-yl)amino)-5-(3-(3-methyl-2-oxoimidazolidin-1-yl)piperidin-1-yl)pyrazine-2-carboxamide

Tert-butyl (R)-3-(6-((3-carbamoyl-6-(3-(3-methyl-2-oxoimidazolidin-1-yl)piperidin-1-yl)pyrazin-2-yl)amino)-3,4-dihydroisoquinolin-2(1H)-yl)azetidine-1-carboxylate was dissolved in DCM:TFA (5:1 ratio, 0.2M) and the reaction was stirred for 4 h. The reaction mixture was concentrated followed by suspension in diethyl ether. This suspension was sonicated, followed by concentration and drying for 16 h to afford (R)-3-((2-(azetidin-3-yl)-1,2,3,4-tetrahydroisoquinolin-6-yl)amino)-5-(3-(3-methyl-2-oxoimidazolidin-1-yl)piperidin-1-yl)pyrazine-2-carboxamide (95% yield). LCMS C₂₆H₃₅N₉O₂ requires: 505, found: m/z=506 [M+H]⁺.

### Example 9: Synthesis of (R)-3-((4-(1-(azetidin-3-yl)piperidin-4-yl)phenyl)amino)-5-(3-(3-methyl-2-oxoimidazolidin-1-yl)piperidin-1-yl)pyrazine-2-carboxamide

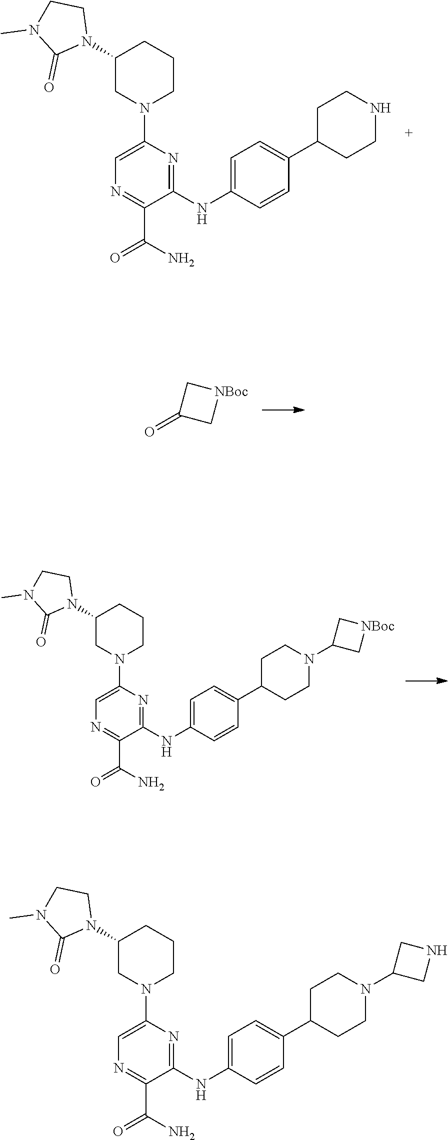 [SMILES: CN1CCN([C@@H]2CCCN(c3cnc(C(N)=O)c(Nc4ccc(C5CCNCC5)cc4)n3)C2)C1=O ; CC(C)(C)OC(=O)N1CC(=O)C1 ; CN1CCN([C@@H]2CCCN(c3cnc(C(N)=O)c(Nc4ccc(C5CCN(C6CN(C(=O)OC(C)(C)C)C6)CC5)cc4)n3)C2)C1=O ; CN1CCN([C@@H]2CCCN(c3cnc(C(N)=O)c(Nc4ccc(C5CCN(C6CNC6)CC5)cc4)n3)C2)C1=O]

The amine intermediate was combined with tert-butyl 3-oxoazetidine-1-carboxylate (1 equiv.) and stirred in a solution of DCE and TEA (10:1, 0.1M) for 5 min. Sodium triacetoxyborohydride (5 equiv.) was then added, and the reaction was stirred at room temperature for 5 h. The reaction was then partitioned between ethyl acetate and water. The organic layer was separated, washed with brine, then dried over magnesium sulfate and filtered. The crude intermediate was then dissolved in DCM:TFA (5:1 ratio, 0.2M) and the reaction was stirred for 4 h. The reaction mixture was concentrated by rotary evaporator, followed by suspension in diethyl ether. This suspension was sonicated, followed by concentration by rotary evaporator and further drying for 16 h to afford tert-butyl 3-oxoazetidine-1-carboxylate (95% over 2 steps). LCMS C₄₁H₄₇N₁₁O₆ requires: 789.9, found: m/z=790.7 [M+H]⁺.

### Example 10: Synthesis of (R)-3-((3-aminophenyl)amino)-5-(3-(3-methyl-2-oxoimidazolidin-1-yl)piperidin-1-yl)pyrazine-2-carboxamide

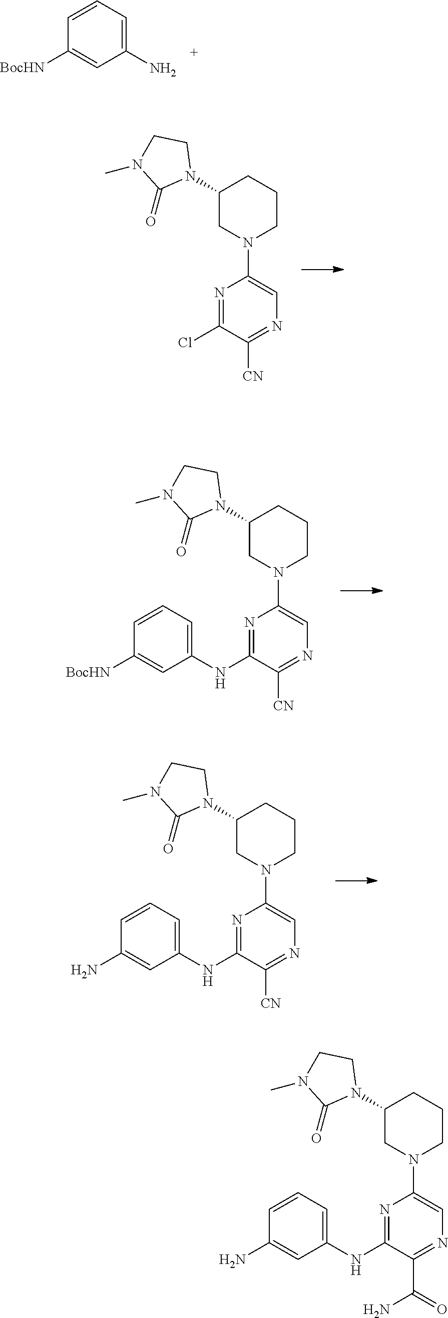 [SMILES: CC(C)(C)OC(=O)Nc1cccc(N)c1 ; CN1CCN([C@@H]2CCCN(c3cnc(C#N)c(Cl)n3)C2)C1=O ; CN1CCN([C@@H]2CCCN(c3cnc(C#N)c(Nc4cccc(NC(=O)OC(C)(C)C)c4)n3)C2)C1=O ; CN1CCN([C@@H]2CCCN(c3cnc(C#N)c(Nc4cccc(N)c4)n3)C2)C1=O ; CN1CCN([C@@H]2CCCN(c3cnc(C(N)=O)c(Nc4cccc(N)c4)n3)C2)C1=O]

### Step 1: tert-butyl (R)-(3-((3-cyano-6-(3-(3-methyl-2-oxoimidazolidin-1-yl)piperidin-1-yl)pyrazin-2-yl)amino)phenyl)carbamate

(R)-3-chloro-5-(3-(3-methyl-2-oxoimidazolidin-1-yl)piperidin-1-yl)pyrazine-2-carbonitrile, tert-butyl (3-aminophenyl)carbamate (1 equiv.), Pd(OAc)₂ (0.15 equiv.), BINAP (0.15 equiv.), and cesium carbonate (2 equiv.) were combined in a microwave tube, followed by addition of dioxane (0.25 M). Nitrogen was bubbled through for 30 seconds, followed by capping. The mixture was allowed to stir at 90° C. for 3 h. The reaction was then cooled, and filtered through Celite, washing with ethyl acetate/methanol. The crude material was purified by MPLC (0-10% MeOH in CH₂Cl₂) to afford tert-butyl (R)-(3-((3-cyano-6-(3-(3-methyl-2-oxoimidazolidin-1-yl)piperidin-1-yl)pyrazin-2-yl)amino)phenyl)carbamate (45% yield). LCMS C₂₅H₃₂N₈O₃ requires: 492.6, found: m/z=493.6 [M+H]⁺.

### Step 2: (R)-3-((3-aminophenyl)amino)-5-(3-(3-methyl-2-oxoimidazolidin-1-yl)piperidin-1-yl)pyrazine-2-carbonitrile

Tert-butyl (R)-(3-((3-cyano-6-(3-(3-methyl-2-oxoimidazolidin-1-yl)piperidin-1-yl)pyrazin-2-yl)amino)phenyl)carbamate was dissolved in DCM:TFA (5:1 ratio, 0.2M) and the reaction was stirred for 4 h. The reaction mixture was concentrated by rotary evaporator, followed by suspension in diethyl ether. This suspension was sonicated, followed by concentration by rotary evaporator and further drying for 16 h to afford (R)-3-((3-aminophenyl)amino)-5-(3-(3-methyl-2-oxoimidazolidin-1-yl)piperidin-1-yl)pyrazine-2-carbonitrile (100% crude yield). LCMS C₂₀H₂₄N₈O requires: 392.5, found: m/z=393.5 [M+H]⁺.

### Step 3: (R)-3-((3-aminophenyl)amino)-5-(3-(3-methyl-2-oxoimidazolidin-1-yl)piperidin-1-yl)pyrazine-2-carboxamide

(R)-3-((3-aminophenyl)amino)-5-(3-(3-methyl-2-oxoimidazolidin-1-yl)piperidin-1-yl)pyrazine-2-carbonitrile was dissolved in methanol/DMSO (10:1) and a pellet of NaOH was added. The reaction was stirred for 5 min, followed by addition of 35% peroxide solution (2 mL of solution per mmol of reactant). This reaction mixture was stirred for 3 h, then partitioned between ethyl acetate and water. The organic layer was separated, dried over magnesium sulfate and concentrated under vacuum, and purified by MPLC (0-10% methanol in DCM) to provide (R)-3-((3-aminophenyl)amino)-5-(3-(3-methyl-2-oxoimidazolidin-1-yl)piperidin-1-yl)pyrazine-2-carboxamide. LCMS C₂₀H₂₆N₈O₂ requires: 410.5, found: m/z=411.5 [M+H]⁺.

### Example 11: Synthesis of (R)-3-((4-(3-aminopropoxy)phenyl)amino)-5-(3-(3-methyl-2-oxoimidazolidin-1-yl)piperidin-1-yl)pyrazine-2-carboxamide

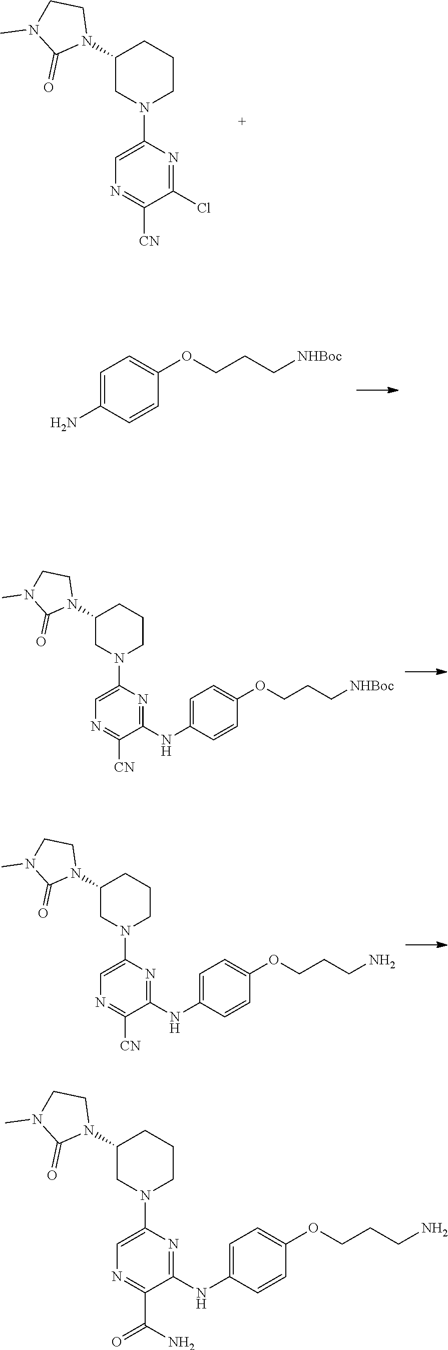 [SMILES: CN1CCN([C@@H]2CCCN(c3cnc(C#N)c(Cl)n3)C2)C1=O ; CC(C)(C)OC(=O)NCCCOc1ccc(N)cc1 ; CN1CCN([C@@H]2CCCN(c3cnc(C#N)c(Nc4ccc(OCCCNC(=O)OC(C)(C)C)cc4)n3)C2)C1=O ; CN1CCN([C@@H]2CCCN(c3cnc(C#N)c(Nc4ccc(OCCCN)cc4)n3)C2)C1=O ; CN1CCN([C@@H]2CCCN(c3cnc(C(N)=O)c(Nc4ccc(OCCCN)cc4)n3)C2)C1=O]

### Step 1: tert-butyl (R)-(3-(4-((3-cyano-6-(3-(3-methyl-2-oxoimidazolidin-1-yl)piperidin-1-yl)pyrazin-2-yl)amino)phenoxy)propyl)carbamate

(R)-3-chloro-5-(3-(3-methyl-2-oxoimidazolidin-1-yl)piperidin-1-yl)pyrazine-2-carbonitrile, tert-butyl (3-(4-aminophenoxy)propyl)carbamate (1 equiv), Pd(OAc)₂ (0.15 equiv), BINAP (0.15 equiv), and cesium carbonate (2 equiv) were combined in a microwave tube, followed by addition of dioxane (0.25 M). Nitrogen was bubbled through for 30 seconds, followed by capping. The mixture was allowed to stir at 90° C. for 3 h. The reaction was then cooled, and filtered through Celite, washing with ethyl acetate/methanol. The crude material was purified by MPLC (0-10% MeOH in CH₂Cl₂) to afford tert-butyl (R)-(3-(4-((3-cyano-6-(3-(3-methyl-2-oxoimidazolidin-1-yl)piperidin-1-yl)pyrazin-2-yl)amino)phenoxy)propyl)carbamate (52% yield). LCMS C₂₈H₃₈N₈O₄ requires: 550, found: m/z=551.7 [M+H]⁺.

### Step 2: (R)-3-((4-(3-aminopropoxy)phenyl)amino)-5-(3-(3-methyl-2-oxoimidazolidin-1-yl)piperidin-1-yl)pyrazine-2-carbonitrile

Tert-butyl (R)-(3-(4-((3-cyano-6-(3-(3-methyl-2-oxoimidazolidin-1-yl)piperidin-1-yl)pyrazin-2-yl)amino)phenoxy)propyl)carbamate was dissolved in DCM:TFA (5:1 ratio, 0.2M) and the reaction was stirred for 4 h. The reaction mixture was concentrated by rotary evaporator, followed by suspension in diethyl ether. This suspension was sonicated, followed by concentration by rotary evaporator and further drying for 16 h to afford (R)-3-((4-(3-aminopropoxy)phenyl)amino)-5-(3-(3-methyl-2-oxoimidazolidin-1-yl)piperidin-1-yl)pyrazine-2-carbonitrile (100% crude yield). LCMS C₂₃H₃₀N₈O₂ requires: 450.6, found: m/z=451.6 [M+H]⁺.

### Step 3: (R)-3-((4-(3-aminopropoxy)phenyl)amino)-5-(3-(3-methyl-2-oxoimidazolidin-1-yl)piperidin-1-yl)pyrazine-2-carboxamide

(R)-3-((4-(3-aminopropoxy)phenyl)amino)-5-(3-(3-methyl-2-oxoimidazolidin-1-yl)piperidin-1-yl)pyrazine-2-carbonitrile was dissolved in methanol/DMSO (10:1) and a pellet of NaOH was added. The reaction was stirred for 5 minutes, followed by addition of 35% peroxide solution (2 mL of solution per mmol of reactant). This reaction mixture was stirred for 3 h, then partitioned between ethyl acetate and water. The organic layer was separated, dried over magnesium sulfate and concentrated under vacuum, and purified by MPLC (0-10% methanol in DCM) to provide (R)-3-((4-(3-aminopropoxy)phenyl)amino)-5-(3-(3-methyl-2-oxoimidazolidin-1-yl)piperidin-1-yl)pyrazine-2-carboxamide (77% yield). LCMS C₂₃H₃₂N₈O₃ requires: 468.6, found: m/z=469.6 [M+H]⁺.

### Example 12: General Synthetic Scheme for Examples 12A-12G

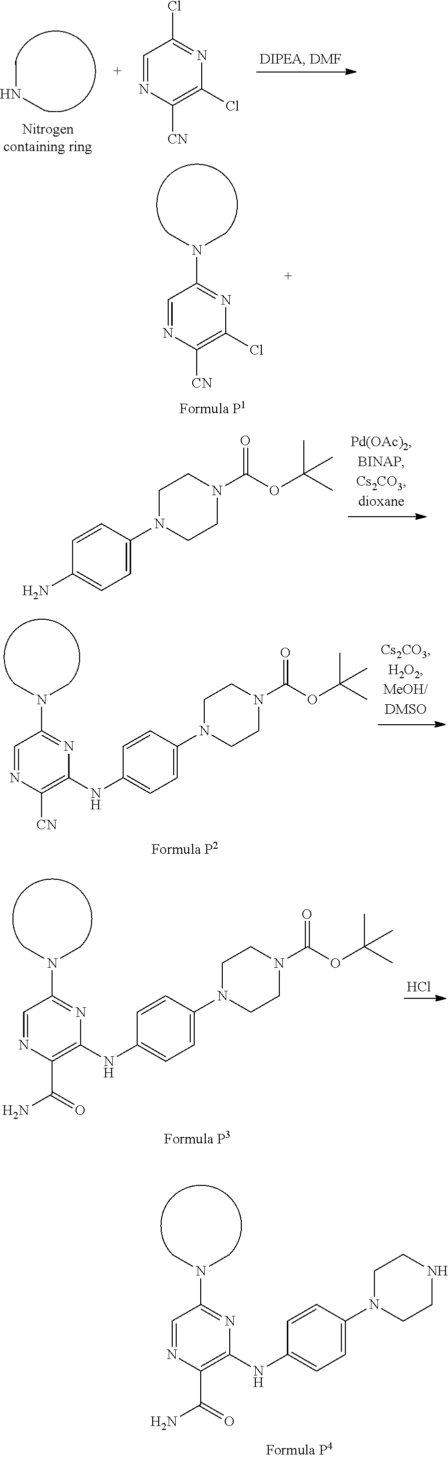

Step 1: Coupling of a Nitrogen Containing Ring to Provide a Compound of Formula P¹.

In a typical synthesis, a mixture of 3,5-dichloropyrazine-2-carbonitrile (˜1 eq.), a cyclic compound containing a ring nitrogen (˜1 eq.), ethylbis(propan-2-yl)amine (˜2 eq.) and a polar aprotic solvent such as DMF was allowed to stir at r.t. for 2 h. An organic solvent such as EtOAc was then added to the reaction mixture, and then washed with water. The organic layer was dried with MgSO₄, filtered, concentrated and purified by a procedure such as crystallization or MPLC (0-100% EtOAc in hexanes) to afford the purified product.

Step 2: Coupling of the Aniline Derivative to Provide a Compound of Formula P².

In a typical synthesis, a mixture of a compound of Formula P¹ (˜1 eq.), tert-butyl 4-(4-aminophenyl)piperazine-1-carboxylate (˜1 eq.), Pd(OAc)₂ (˜0.3 eq.), BINAP (˜0.3 eq.) and Cs₂CO₃ (˜3 eq.) was degassed and backfilled with N₂ 5 times. The mixture was allowed to stir at about 100° C. for about 90 min. The mixture was filtered, e.g. filtered through celite, the cake was washed with a solvent system such as MeOH/EtOAc. The crude product was then concentrated and purified using a technique such as crystallization or MPLC.

Step 3: Conversion of the Cyano Group to Acetamide to Produce a Compound of Formula P³.

H₂O₂(30% in water; ˜0.17 eq.) was added to a mixture of a compound of Formula P² (˜1 eq.), Cs₂CO₃ (˜1 eq.), and a mixture of 36:1 MeOH:DMSO as the solvent. The mixture was allowed to stir at r.t. for about 30 min. The mixture was concentrated and diluted with an organic solvent such as EtOAc, and the organic phase was washed with water, and then brine. The organic layer was further dried with MgSO₄, filtered, concentrated and purified using a technique such as crystallization or MPLC.

### Step 4: Removal of the BOC Group to Provide a Compound of Formula P⁴

In a typical synthesis, a mixture of a compound of Formula P³ was dissolved in a 2.5:1 mixture of 4M hydrogen chloride in dioxane to THF, and was allowed to stir at r.t. for 2 h. The volatiles were removed to afford the product.

### Example 12A: Synthesis of 3-((4-(piperazin-1-yl)phenyl)amino)-5-(piperidin-1-yl)pyrazine-2-carboxamide

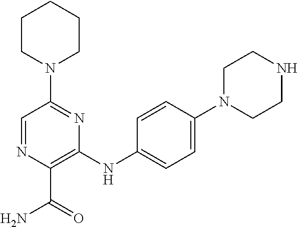 [SMILES: NC(=O)c1ncc(N2CCCCC2)nc1Nc1ccc(N2CCNCC2)cc1]

### Step 1: 3-chloro-5-(piperidin-1-yl)pyrazine-2-carbonitrile

A mixture of 3,5-dichloropyrazine-2-carbonitrile (850 mg, 4.89 mmol), piperidine (0.48 mL, 4.89 mmol), ethylbis(propan-2-yl)amine (1.70 mL, 9.77 mmol) and DMF (20 mL) was allowed to stir at r.t. for 2 h. EtOAc and H₂O were added. The organic layer was dried with MgSO₄, filtered, concentrated and purified by MPLC (0-100% EtOAc in hexanes) to afford 3-chloro-5-(piperidin-1-yl)pyrazine-2-carbonitrile (1079.6 mg, 99.2%). LCMS: C₁₀H₁₁ClN₄ requires: 222, found: m/z=223 [M+H]⁺.

### Step 2: tert-butyl 4-(4-{[3-cyano-6-(piperidin-1-yl)pyrazin-2-yl]amino}phenyl)piperazine-1-carboxylate

A mixture of 3-chloro-5-(piperidin-1-yl)pyrazine-2-carbonitrile (534 mg, 2.40 mmol), tert-butyl 4-(4-aminophenyl)piperazine-1-carboxylate (665 mg, 2.40 mmol), Pd(OAc)₂ (177 mg, 0.79 mmol), BINAP (493 mg, 0.79 mmol) and Cs₂CO₃ (2345 mg, 7.20 mmol) was degassed and backfilled with N₂ 5 times. The mixture was allowed to stir at 100° C. for 90 min. The mixture was filtered through celite washing with MeOH/EtOAc, concentrated and purified by MPLC (0-100% EtOAc in CH₂C₁₂) to afford tert-butyl 4-(4-{[3-cyano-6-(piperidin-1-yl)pyrazin-2-yl]amino}phenyl)piperazine-1-carboxylate (833 mg, 74.9%). LCMS: C₂₅H₃₃N₇O₂ requires: 463, found: m/z=464 [M+H]⁺.

### Step 3: tert-butyl 4-(4-((3-carbamoyl-6-(piperidin-1-yl)pyrazin-2-yl)amino)phenyl)piperazine-1-carboxylate

H₂O₂(30% in water, 3.03 mL, 0.30 mmol) was added to a mixture of tert-butyl 4-(4-{[3-cyano-6-(piperidin-1-yl)pyrazin-2-yl]amino}phenyl)piperazine-1-carboxylate (833 mg, 1.80 mmol), Cs₂CO₃ (586 mg, 1.80 mmol), MeOH (35 mL) and DMSO (1 mL). The mixture was allowed to stir at r.t. for 30 min. The mixture was concentrated. EtOAc was added and the organic phase was washed with H₂O and brine. The organic layer was dried with MgSO₄, filtered, concentrated and purified by MPLC (0-10% MeOH in CH₂Cl₂) to afford tert-butyl 4-(4-((3-carbamoyl-6-(piperidin-1-yl)pyrazin-2-yl)amino)phenyl)piperazine-1-carboxylate (809 mg, 93.5%). LCMS: C₂₅H₃₅N₇O₃ requires: 481, found: m/z=482 [M+H]⁺.

### Step 4: 3-{[4-(piperazin-1-yl)phenyl]amino}-5-(piperidin-1-yl)pyrazine-2-carboxamide

A mixture of tert-butyl 4-(4-{[3-carbamoyl-6-(piperidin-1-yl)pyrazin-2-yl]amino}phenyl)piperazine-1-carboxylate (20 mg, 0.04 mmol), hydrogen chloride (4M in dioxane, 0.26 mL, 1.04 mmol) and THE (0.1 mL) was allowed to stir at r.t. for 2 h. The volatiles were removed to afford 3-{[4-(piperazin-1-yl)phenyl]amino}-5-(piperidin-1-yl)pyrazine-2-carboxamide (15 mg, 95%). LCMS: C₂₀H₂₇N₇O requires: 381, found: m/z=382 [M+H]⁺.

### Example 12B: Synthesis of (R)-5-(3-(hydroxymethyl)piperidin-1-yl)-3-((4-(piperidin-4-yl)phenyl)amino)pyrazine-2-carboxamide

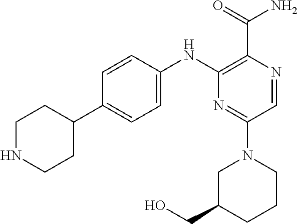 [SMILES: NC(=O)c1ncc(N2CCC[C@@H](CO)C2)nc1Nc1ccc(C2CCNCC2)cc1]

Step 1: (R)-3-chloro-5-(3-(hydroxymethyl)piperidin-1-yl)pyrazine-2-carbonitrile using (R)-piperidin-3-ylmethanol as the amine. LCMS C₁₁H₁₃ClN₄O requires: 252 found: m/z=253 [M+H]⁺.

Step 2: tert-butyl (R)-4-(4-((3-cyano-6-(3-(hydroxymethyl)piperidin-1-yl)pyrazin-2-yl)amino)phenyl)piperidine-1-carboxylate. LCMS: C₂₇H₃₆N₆O₃ requires: 492 found: m/z=493 [M+H]⁺.

Step 3: tert-butyl (R)-4-(4-((3-carbamoyl-6-(3-(hydroxymethyl)piperidin-1-yl)pyrazin-2-yl)amino)phenyl)piperidine-1-carboxylate. LCMS C₂₇H₃₈N₆O₄ requires: 510 found: m/z=511 [M+H]⁺.

Step 4: (R)-5-(3-(hydroxymethyl)piperidin-1-yl)-3-((4-(piperidin-4-yl)phenyl)amino)pyrazine-2-carboxamide. LCMS C₂₂H₃₀N₆O₂ requires: 410 found: m/z=411 [M+H]⁺.

### Example 12C: Synthesis of 5-(4,4-difluoro-3-(hydroxymethyl)piperidin-1-yl)-3-((4-(piperidin-4-yl)phenyl)amino)pyrazine-2-carboxamide

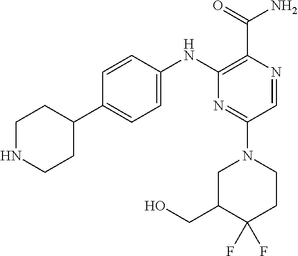 [SMILES: NC(=O)c1ncc(N2CCC(F)(F)C(CO)C2)nc1Nc1ccc(C2CCNCC2)cc1]

Step 1: 3-chloro-5-(4,4-difluoro-3-(hydroxymethyl)piperidin-1-yl)pyrazine-2-carbonitrile using (4,4-difluoropiperidin-3-yl)methanol as the amine. LCMS C₁₁H₁₁ClF₂N₄O requires: 288 found: m/z=289 [M+H]⁺.

Step 2: tert-butyl 4-(4-((3-cyano-6-(4,4-difluoro-3-(hydroxymethyl)piperidin-1-yl)pyrazin-2-yl)amino)phenyl)piperidine-1-carboxylate. LCMS C₂₇H₃₄F₂N₆O₃ requires: 528 found: m/z=529 [M+H]⁺.

Step 3: tert-butyl 4-(4-((3-carbamoyl-6-(4,4-difluoro-3-(hydroxymethyl)piperidin-1-yl)pyrazin-2-yl)amino)phenyl)piperidine-1-carboxylate. LCMS C₂₇H₃₆F₂N₆O₄ requires: 546 found: m/z=547 [M+H]⁺.

Step 4: 5-(4,4-difluoro-3-(hydroxymethyl)piperidin-1-yl)-3-((4-(piperidin-4-yl)phenyl)amino)pyrazine-2-carboxamide. LCMS C₂₂H₂₈F₂N₆O₂ requires: 446 found: m/z=447 [M+H]⁺.

### Example 12D: Synthesis of 3-((4-(piperidin-4-yl)phenyl)amino)-5-(2-oxa-6-azaspiro[3.5]nonan-6-yl)pyrazine-2-carboxamide

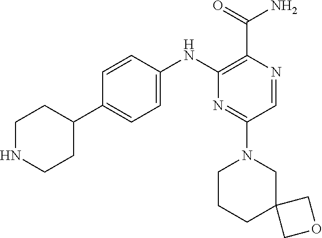 [SMILES: NC(=O)c1ncc(N2CCCC3(COC3)C2)nc1Nc1ccc(C2CCNCC2)cc1]

Step 1: 3-chloro-5-(2-oxa-6-azaspiro[3.5]nonan-6-yl)pyrazine-2-carbonitrile using 2-oxa-6-azaspiro[3.5]nonane as the amine. LCMS C₁₂H₁₃ClN₄O requires: 264 found: m/z=265 [M+H]⁺.
Step 2: tert-butyl 4-(4-((3-cyano-6-(2-oxa-6-azaspiro[3.5]nonan-6-yl)pyrazin-2-yl)amino)phenyl)piperidine-1-carboxylate. LCMS C₂₈H₃₆N₆O₃ requires: 504 found: m/z=505 [M+H]⁺.
Step 3: tert-butyl 4-(4-((3-carbamoyl-6-(2-oxa-6-azaspiro[3.5]nonan-6-yl)pyrazin-2-yl)amino)phenyl)piperidine-1-carboxylate. LCMS C₂₈H₃₈N₆O₄ requires: 522 found: m/z=523 [M+H]⁺.

Step 4: 3-((4-(piperidin-4-yl)phenyl)amino)-5-(2-oxa-6-azaspiro[3.5]nonan-6-yl)pyrazine-2-carboxamide. LCMS C₂₃H₃₀N₆O₂ requires: 422 found: m/z=423 [M+H]⁺.

### Example 12E: Synthesis of 3-((4-(piperidin-4-yl)phenyl)amino)-5-(2-oxa-6-azaspiro[3.4]octan-6-yl)pyrazine-2-carboxamide

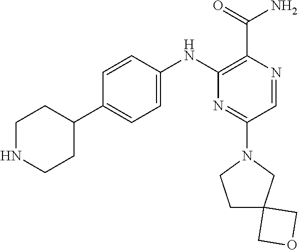 [SMILES: NC(=O)c1ncc(N2CCC3(COC3)C2)nc1Nc1ccc(C2CCNCC2)cc1]

Step 1: 3-chloro-5-(2-oxa-6-azaspiro[3.4]octan-6-yl)pyrazine-2-carbonitrile using 2-oxa-6-azaspiro[3.4]octane as the amine. LCMS CnHnClN₄O requires: 250 found: m/z=251 [M+H]⁺.

Step 2: tert-butyl 4-(4-((3-cyano-6-(2-oxa-6-azaspiro[3.4]octan-6-yl)pyrazin-2-yl)amino)phenyl)piperidine-1-carboxylate. LCMS C₂₇H₃₄N₆O₃ requires: 490 found: m/z=491 [M+H]⁺.

Step 3: tert-butyl 4-(4-((3-carbamoyl-6-(2-oxa-6-azaspiro[3.4]octan-6-yl)pyrazin-2-yl)amino)phenyl)piperidine-1-carboxylate. LCMS C₂₇H₃₆N₆O₄ requires: 508 found: m/z=509 [M+H]⁺.

Step 4: 3-((4-(piperidin-4-yl)phenyl)amino)-5-(2-oxa-6-azaspiro[3.4]octan-6-yl)pyrazine-2-carboxamide. LCMS C₂₂H₂₈N₆O₂ requires: 408 found: m/z=409 [M+H]⁺.

### Example 12F: Synthesis of 3-((4-(piperidin-4-yl)phenyl)amino)-5-(1H-pyrazol-1-yl)pyrazine-2-carboxamide

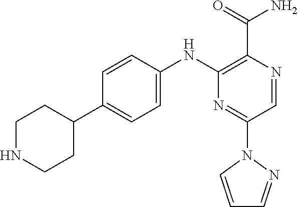 [SMILES: NC(=O)c1ncc(-n2cccn2)nc1Nc1ccc(C2CCNCC2)cc1]

Step 1: 3-chloro-5-(1H-pyrazol-1-yl)pyrazine-2-carbonitrile using pyrazole as the amine. LCMS C₈H₄ClN₅ requires: 205 found: m/z=206 [M+H]⁺.

Step 2: tert-butyl 4-(4-((3-cyano-6-(1H-pyrazol-1-yl)pyrazin-2-yl)amino)phenyl)piperidine-1-carboxylate. LCMS C₂₄H₂₇N₇O₂ requires: 445 found: m/z=446 [M+H]⁺.

Step 3: tert-butyl 4-(4-((3-carbamoyl-6-(1H-pyrazol-1-yl)pyrazin-2-yl)amino)phenyl)piperidine-1-carboxylate. LCMS C₂₄H₂₉N₇O₃ requires: 463 found: m/z=464 [M+H]⁺.

Step 4: 3-((4-(piperidin-4-yl)phenyl)amino)-5-(1H-pyrazol-1-yl)pyrazine-2-carboxamide. LCMS C₁₉H₂₁N₇O requires: 363 found: m/z=364 [M+H]⁺.

### Example 12G: Synthesis of 3-((4-(piperidin-4-yl)phenyl)amino)-5-(3-(trifluoromethyl)-1H-pyrazol-1-yl)pyrazine-2-carboxamide

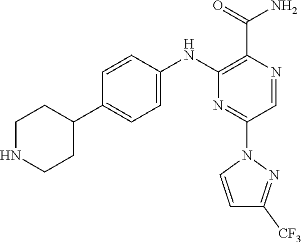 [SMILES: NC(=O)c1ncc(-n2ccc(C(F)(F)F)n2)nc1Nc1ccc(C2CCNCC2)cc1]

Step 1: 3-chloro-5-(3-(trifluoromethyl)-1H-pyrazol-1-yl)pyrazine-2-carbonitrile using 3-trifluoromethylpyrazole as the amine. LCMS C₉H₃C₁F₃N₅ requires: 273 found: m/z=274 [M+H]⁺.

Step 2: tert-butyl 4-(4-((3-cyano-6-(3-(trifluoromethyl)-1H-pyrazol-1-yl)pyrazin-2-yl)amino)phenyl)piperidine-1-carboxylate. LCMS C₂₅H₂₆F₃N₇O₂ requires: 513 found: m/z=514 [M+H]⁺.

Step 3: tert-butyl 4-(4-((3-carbamoyl-6-(3-(trifluoromethyl)-1H-pyrazol-1-yl)pyrazin-2-yl)amino)phenyl)piperidine-1-carboxylate. LCMS C₂₅H₂₈F₃N₇O₃ requires: 531 found: m/z=532 [M+H]⁺.

Step 4: 3-((4-(piperidin-4-yl)phenyl)amino)-5-(3-(trifluoromethyl)-1H-pyrazol-1-yl)pyrazine-2-carboxamide. LCMS C₂₀H₂₀F₃N₇O requires: 431 found: m/z=432 [M+H]⁺.

### Example 13: Synthesis of (R)-4-((3-carbamoyl-6-(3-(3-methyl-2-oxoimidazolidin-1-yl)piperidin-1-yl)pyrazin-2-yl)amino)benzoic acid

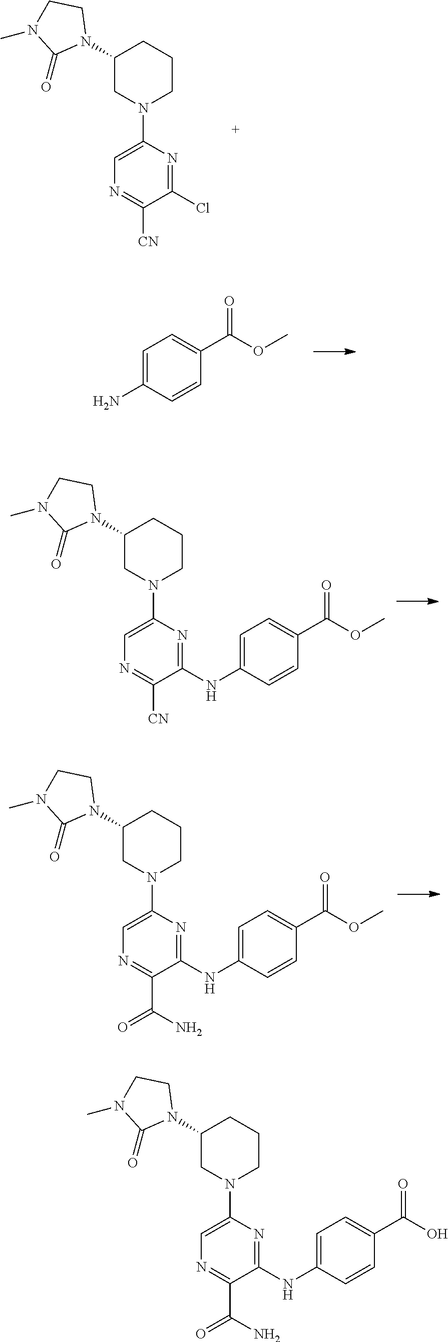

### Step 1: methyl (R)-4-((3-cyano-6-(3-(3-methyl-2-oxoimidazolidin-1-yl)piperidin-1-yl)pyrazin-2-yl)amino)benzoate

(R)-3-chloro-5-(3-(3-methyl-2-oxoimidazolidin-1-yl)piperidin-1-yl)pyrazine-2-carbonitrile, methyl 4-aminobenzoate (1 equiv.), Pd(OAc)₂ (0.15 equiv.), BINAP (0.15 equiv.), and cesium carbonate (2 equiv.) were combined in a microwave tube, followed by addition of dioxane (0.25 M). Nitrogen was bubbled through for 30 seconds, followed by capping. The mixture was allowed to stir at 90° C. for 3 h. The reaction was then cooled, and filtered through Celite, washing with ethyl acetate/methanol. The crude material was purified by MPLC (0-10% MeOH in CH₂Cl₂) to afford methyl (R)-4-((3-cyano-6-(3-(3-methyl-2-oxoimidazolidin-1-yl)piperidin-1-yl)pyrazin-2-yl)amino)benzoate (74% yield). LCMS C₂₂H₂₅N₇O₃ requires: 435.5, found: m/z=436.6 [M+H]⁺.

### Step 2: methyl (R)-4-((3-carbamoyl-6-(3-(3-methyl-2-oxoimidazolidin-1-yl)piperidin-1-yl)pyrazin-2-yl)amino)benzoate

Methyl (R)-4-((3-cyano-6-(3-(3-methyl-2-oxoimidazolidin-1-yl)piperidin-1-yl)pyrazin-2-yl)amino)benzoate was dissolved in methanol/DMSO (10:1) and a pellet of NaOH was added. The reaction was stirred for 5 min, followed by addition of 35% peroxide solution (2 mL of solution per mmol of reactant). This reaction mixture was stirred for 3 h, then partitioned between ethyl acetate and water. The organic layer was separated and dried over magnesium sulfate. Chromatography (0-10% methanol in DCM) provided methyl (R)-4-((3-carbamoyl-6-(3-(3-methyl-2-oxoimidazolidin-1-yl)piperidin-1-yl)pyrazin-2-yl)amino)benzoate in 48% yield. LCMS C₂₂H₂₇N₇O₄ requires: 453.5, found: m/z=454.6 [M+H]⁺.

### Step 3: (R)-4-((3-carbamoyl-6-(3-(3-methyl-2-oxoimidazolidin-1-yl)piperidin-1-yl)pyrazin-2-yl)amino)benzoic acid

Starting material was dissolved in THE (0.1 M) followed by addition of 2N LiGH (aq, 25% by volume of THF). The reaction was stirred at 80° C. for 4 h. The reaction was then poured into ethyl acetate/2N HCl in a separatory funnel. The organic layer was separated, and the aqueous layer was further extracted with methylene chloride/methanol (10%). Both organic layers were dried over magnesium sulfate, filtered, and concentrated to provide (R)-4-((3-carbamoyl-6-(3-(3-methyl-2-oxoimidazolidin-1-yl)piperidin-1-yl)pyrazin-2-yl)amino)benzoic acid (88% yield) with no further purification. LCMS C₂₁H₂₅N₇O₄ requires: 439.5, found: m/z=440.6 [M+H]⁺.

### Example 14: General Procedure A

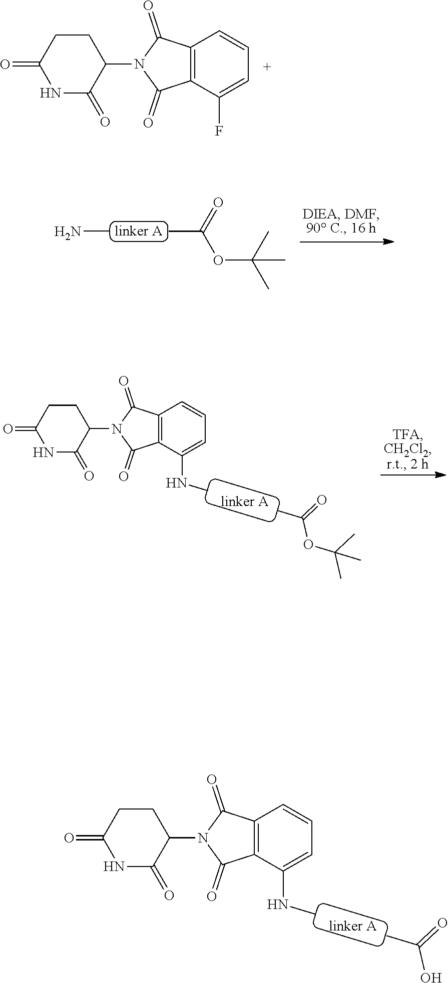

As used in General Procedure A, “linker A” is —X²—X³—X⁴—X⁵—, wherein each of X², X³, X⁴, and X⁵ are defined above for the compound of Formula (A).

Step 1: A mixture of 2-(2,6-dioxopiperidin-3-yl)-4-fluoro-2,3-dihydro-1H-isoindole-1,3-dione (0.26 mmol), aminoester (0.26 mmol), ethylbis(propan-2-yl)amine (0.52 mmol) and DMF (1 mL) was allowed to stir at 90° C. overnight. The mixture was cooled and purified by HPLC (5-95% MeCN in H₂O with 0.1% TFA) to afford the tert-butylester intermediate.

Step 2: A mixture of tert-butyl 4-{[2-(2,6-dioxopiperidin-3-yl)-1,3-dioxo-2,3-dihydro-1H-isoindol-4-yl]amino}butanoate (0.10 mmol), CH₂Cl₂ (1 mL), and TFA (1 mL) was allowed to stir at r.t. for 2 h. The mixture was concentrated to afford the carboxylic acid product.

### Example 14A: Synthesis of 3-(2-((2-(2,6-dioxopiperidin-3-yl)-1,3-dioxoisoindolin-4-yl)amino)ethoxy)propanoic acid

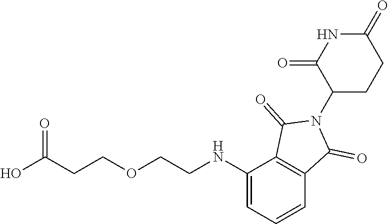 [SMILES: O=C(O)CCOCCNc1cccc2c1C(=O)N(C1CCC(=O)NC1=O)C2=O]

### Step 1 product: tert-butyl 3-[2-[[2-(2,6-dioxo-3-piperidyl)-1,3-dioxo-isoindolin-4-yl]amino]ethoxy]propanoate (1.8 g, 51.9%). LCMS; C₂₂H₂₇N₃O₇ requires: 445, found: m/z=468 [M+Na]⁺

Step 2 product: 3-[2-[[2-(2,6-dioxo-3-piperidyl)-1,3-dioxo-isoindolin-4-yl]amino]ethoxy]propanoic acid (526.8 mg, 32%). LCMS; C₁₈H₁₉N₃O₇ requires: 389, found: m/z=390 [M+H]⁺.

### Example 14B: Synthesis of 3-(2-(2-(2-((2-(2,6-dioxopiperidin-3-yl)-1,3-dioxoisoindolin-4-yl)amino)ethoxy)ethoxy)ethoxy)propanoic acid

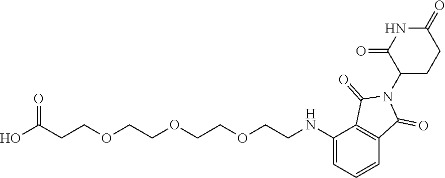 [SMILES: O=C(O)CCOCCOCCOCCNc1cccc2c1C(=O)N(C1CCC(=O)NC1=O)C2=O]

Step 1 product: tert-butyl 3-[2-[2-[2-[[2-(2,6-dioxo-3-piperidyl)-1,3-dioxoisoindolin-4-yl]amino]ethoxy]ethoxy]ethoxy]propanoate (1.6 g, 41%). LCMS; C₂₆H₃₅N₃O₉ requires: 533, found: m/z=534 [M+H]⁺.

Step 2 product: 3-[2-[2-[2-[[2-(2,6-dioxo-3-piperidyl)-1,3-dioxo-isoindolin-4-yl]amino]ethoxy]ethoxy]ethoxy]propanoic acid (1.2 g, 73.62%). LCMS; C₂₂H₂₇N₃O₉ requires: 477, found: m/z=478 [M+H]⁺.

### Example 14C: Synthesis of trans-4-((2-(2,6-dioxopiperidin-3-yl)-1,3-dioxoisoindolin-4-yl)amino)cyclohexane-1-carboxylic acid

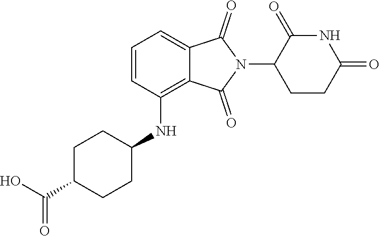 [SMILES: O=C1CCC(N2C(=O)c3cccc(N[C@H]4CC[C@H](C(=O)O)CC4)c3C2=O)C(=O)N1]

Step 1 product: trans-tert-butyl 4-((2-(2,6-dioxopiperidin-3-yl)-1,3-dioxoisoindolin-4-yl)amino)cyclohexane-1-carboxylate (43.40 mg, 47.0%).

Step 2 product: trans-4-((2-(2,6-dioxopiperidin-3-yl)-1,3-dioxoisoindolin-4-yl)amino)cyclohexane-1-carboxylic acid (38 mg, 99%).

### Example 14D: Synthesis of 3-(3-((2-(2,6-dioxopiperidin-3-yl)-1-oxoisoindolin-4-yl)amino)-3-oxopropoxy)propanoic acid

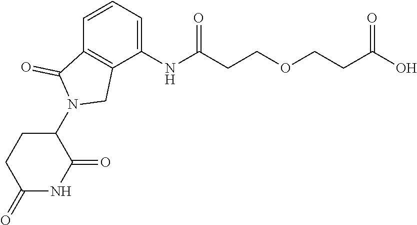 [SMILES: O=C(O)CCOCCC(=O)Nc1cccc2c1CN(C1CCC(=O)NC1=O)C2=O]

Step 1: A mixture of lenalidomide (270 mg, 1.04 mmol), 3-[3-(tert-butoxy)-3-oxopropoxy]propanoic acid (250 mg, 1.15 mmol), HATU (515 mg, 1.35 mmol), ethylbis(propan-2-yl)amine (0.73 mL, 4.17 mmol) and DMF (5 mL) was allowed to stir at rt for 6 h. EtOAc and H₂O were added. The organic layer was dried with MgSO₄, filtered, concentrated and purified by MPLC (20-100% EtOAc in hexanes) to afford tert-butyl 3-(2-{[2-(2,6-dioxopiperidin-3-yl)-1-oxo-3H-isoindol-4-yl]carbamoyl}ethoxy)propanoate (307 mg, 64%). LCMS: C₂₃H₂₉N₃O₇ requires: 459, found: m/z=460 [M+H]⁺.

Step 2: A mixture of tert-butyl 3-(2-{[2-(2,6-dioxopiperidin-3-yl)-1-oxo-3H-isoindol-4-yl]carbamoyl}ethoxy)propanoate (307 mg, 0.67 mmol), CH₂Cl₂ (5 mL), and TFA (1 mL) was allowed to stir at r.t. for 2 h. The mixture was concentrated to afford 3-(3-((2-(2,6-dioxopiperidin-3-yl)-1-oxoisoindolin-4-yl)amino)-3-oxopropoxy)propanoic acid (269 mg, 99%). LCMS: C₁₉H₂₁N₃O₇ requires: 403, found: m/z=404 [M+H]⁺.

### Example 15: General Procedure B

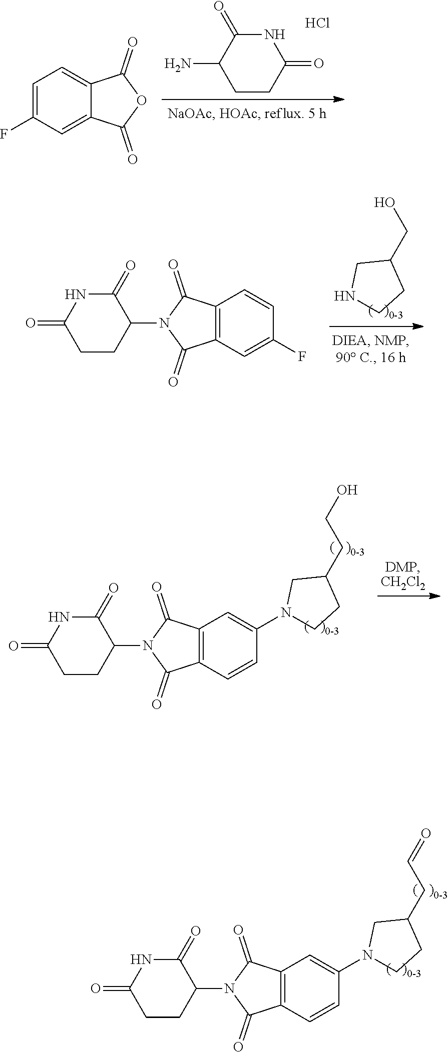

### Step 1: 2-(2,6-dioxopiperidin-3-yl)-5-fluoroisoindoline-1,3-dione

A mixture of 5-fluoro-1,3-dihydro-2-benzofuran-1,3-dione (5.0 g, 30.10 mmol), 3-aminopiperidine-2,6-dione hydrochloride (6.9 g, 42.14 mmol) and NaOAc (4.2 g, 51.17 mmol) in HOAc (50 mL) was stirred at 120° C. for 5 h before concentrated under vacuum. The residue was washed with water and the solid was collected by filtration. The crude product was washed with water twice and ethyl acetate twice and dried under oven to afford 2-(2,6-dioxopiperidin-3-yl)-5-fluoroisoindoline-1,3-dione (7.7 g, 92%) as a light brown solid. ¹H NMR (300 MHz, DMSO-d₆) δ 11.16 (s, 1H), 8.03-8.00 (m, 1H), 7.87-7.85 (m, 1H), 7.75-7.70 (m, 1H), 5.19-5.15 (m, 1H), 2.94-2.86 (m, 1H), 2.63-2.48 (m, 2H), 2.12-2.06 (m, 1H). F NMR (300 MHz, DMSO-d₆) δ-102.078.

### Step 2: Amine Displacement of Aryl Fluoride

To a solution of 2-(2,6-dioxopiperidin-3-yl)-5-fluoro-2,3-dihydro-1H-isoindole-1,3-dione (1.0 g, 3.62 mmol) in N-Methyl pyrrolidone (10 mL) were added the amine (3.60 mmol) and DIEA (1.4 g, 10.83 mmol). The resulting solution was stirred at 80° C. for 16 h. The reaction mixture was cooled down to room temperature and purified by reverse phase flash chromatography to afford the corresponding final product.

### Step 3: Alcohol Oxidation to the Aldehyde

To a mixture of the alcohol (1.06 mmol) in CH₂Cl₂(10 mL) was added Dess-Martin periodinane (2.12 mmol). The mixture was allowed to stir at room temperature for 1 h. The mixture was purified by column chromatography to afford the desired aldehyde.

### Example 15A: Synthesis of 2-(1-(2-(2,6-dioxopiperidin-3-yl)-1,3-dioxoisoindolin-5-yl)piperidin-4-yl)acetaldehyde

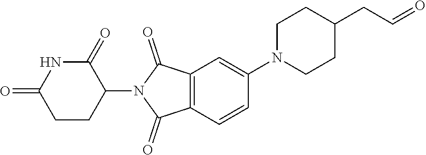 [SMILES: O=CCC1CCN(c2ccc3c(c2)C(=O)N(C2CCC(=O)NC2=O)C3=O)CC1]

Step 2: Followed General Procedure B with 2-(piperidin-4-yl)ethan-1-ol to afford 2-(2,6-dioxopiperidin-3-yl)-5-(4-(2-hydroxyethyl)piperidin-1-yl)isoindoline-1,3-dione (822.8 mg, 59%) as a yellow solid. ¹H NMR (300 MHz, DMSO-d₆) δ 11.09 (s, 1H), 7.65 (d, J 8.4 Hz, 1H), 7.30 (d, J 2.4 Hz, 1H), 7.23 (dd, J 8.4, 2.4 Hz, 1H), 5.07 (dd, J 12.6, 5.4 Hz, 1H), 4.40 (t, J=5.1 Hz, 1H), 4.04 (d, J 13.2 Hz, 2H), 3.64-3.40 (m, 2H), 3.09-2.79 (m, 3H), 2.70-2.51 (m, 2H), 2.07-1.94 (m, 1H), 1.77-1.66 (m, 3H), 1.41-1.34 (m, 2H), 1.24-1.12 (m, 2H). MS (ESI) calc'd for (C₂₀H₂₃N₃O₅) [M+H]⁺, 386.2; found 386.1.

Step 3: 2-(1-(2-(2,6-dioxopiperidin-3-yl)-1,3-dioxoisoindolin-5-yl)piperidin-4-yl)acetaldehyde LCMS C₂₀H₂₁N₃O₅ requires: 383, found: m/z=384 [M+H]⁺.

### Example 15B: Synthesis of 1-(2-(2,6-dioxopiperidin-3-yl)-1,3-dioxoisoindolin-5-yl)azetidine-3-carbaldehyde

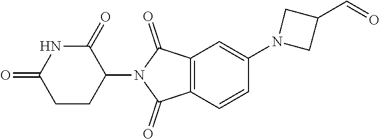 [SMILES: O=CC1CN(c2ccc3c(c2)C(=O)N(C2CCC(=O)NC2=O)C3=O)C1]

Step 2: Followed General Procedure B with azetidin-3-ylmethanol hydrochloride to afford 2-(2,6-dioxopiperidin-3-yl)-5-(3-(hydroxymethyl)azetidin-1-yl)isoindoline-1,3-dione (1.85 g, 68%) as a yellow solid. ¹H NMR (400 MHz, DMSO-d₆) δ 11.09 (s, 1H), 7.63 (d, J 8.4 Hz, 1H), 6.76 (d, J 2.0 Hz, 1H), 6.62 (dd, J 8.4, 2.0 Hz, 1H), 5.06 (dd, J 12.4, 5.2 Hz, 1H), 4.86 (t, J 5.2 Hz, 1H), 4.05 (t, J 8.4 Hz, 2H), 3.77 (dd, J 8.4, 5.2 Hz, 2H), 3.60 (t, J 5.2 Hz, 2H), 3.00-2.81 (m, 2H), 2.65-2.53 (m, 2H), 2.06-1.96 (m, 1H). MS (ESI) calc'd for (C₁₇H₁₇N₃O₅) [M+H]⁺, 344.1; found 344.4.

Step 3: 1-(2-(2,6-dioxopiperidin-3-yl)-1,3-dioxoisoindolin-5-yl)azetidine-3-carbaldehyde LCMS C₁₇H₁₅N₃O₅ requires: 341, found: m/z=343 [M+H]⁺.

### Example 15C: Synthesis of 2-(1-(2-(2,6-dioxopiperidin-3-yl)-1,3-dioxoisoindolin-5-yl)azetidin-3-yl)acetaldehyde

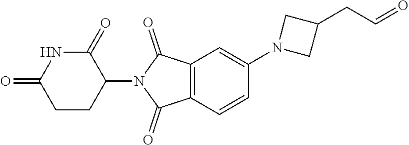 [SMILES: O=CCC1CN(c2ccc3c(c2)C(=O)N(C2CCC(=O)NC2=O)C3=O)C1]

Step 2: Followed General Procedure B with 2-(azetidin-3-yl)ethan-1-ol hydrochloride to afford 2-(2,6-dioxopiperidin-3-yl)-5-(3-(2-hydroxyethyl)azetidin-1-yl)isoindoline-1,3-dione (584.5 mg, 30%) as a yellow solid. ¹H NMR (300 MHz, DMSO-d₆) δ 11.09 (s, 1H), 7.63 (d, J 8.4 Hz, 1H), 6.75 (d, J 2.1 Hz, 1H), 6.62 (dd, J 8.4, 2.1 Hz, 1H), 5.06 (dd, J 12.6, 5.4 Hz, 1H), 4.51 (t, J 5.1 Hz, 1H), 4.14 (t, J 8.1 Hz, 2H), 3.71-3.67 (m, 2H), 3.47-3.40 (m, 2H), 2.99-2.75 (m, 2H), 2.61-2.58 (m, 1H), 2.52-2.46 (m, 1H), 2.10-1.95 (m, 1H), 1.82-1.76 (m, 2H). MS (ESI) calc'd for (C₁₈H₁₉N₃O₅) [M+H]⁺, 358.1; found 358.4.

Step 3: 2-(1-(2-(2,6-dioxopiperidin-3-yl)-1,3-dioxoisoindolin-5-yl)azetidin-3-yl)acetaldehyde LCMS C₁₈H₁₇N₃O₅ requires: 355, found: m/z=356 [M+H]⁺.

### Example 15D: Synthesis of (3R)-1-(2-(2,6-dioxopiperidin-3-yl)-1,3-dioxoisoindolin-5-yl)piperidine-3-carbaldehyde

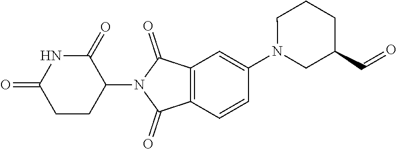 [SMILES: O=C[C@@H]1CCCN(c2ccc3c(c2)C(=O)N(C2CCC(=O)NC2=O)C3=O)C1]

Step 2: Followed General Procedure B with (R)-piperidin-3-ylmethanol hydrochloride to afford 2-(2,6-dioxopiperidin-3-yl)-5-((R)-3-(hydroxymethyl)piperidin-1-yl)isoindoline-1,3-dione (916.3 mg, 45%) as a yellow solid. ¹H NMR (400 MHz, DMSO-d₆/D₂O) δ 7.62 (d, J 8.4 Hz, 1H), 7.22 (d, J 2.4 Hz, 1H), 7.16 (dd, J 8.4, 2.4 Hz, 1H), 4.99 (dd, J 12.8, 5.2 Hz, 1H), 3.98-3.76 (m, 2H), 3.42-3.22 (m, 2H), 3.08-2.90 (m, 1H), 2.89-2.71 (m, 2H), 2.61-2.43 (m, 2H), 2.02-1.99 (m, 1H), 1.73-1.69 (m, 3H), 1.49-1.40 (m, 1H), 1.26-1.18 (m, 1H). MS (ESI) calc'd for (C₁₉H₂₁N₃O₅) [M+H]⁺, 372.1; found 372.4.

Step 3: (3R)-1-(2-(2,6-dioxopiperidin-3-yl)-1,3-dioxoisoindolin-5-yl)piperidine-3-carbaldehyde LCMS C₁₉H₁₉N₃O₅ requires: 369, found: m/z=370 [M+H]⁺.

### Example 15E: Synthesis of (3S)-1-(2-(2,6-dioxopiperidin-3-yl)-1,3-dioxoisoindolin-5-yl)piperidine-3-carbaldehyde

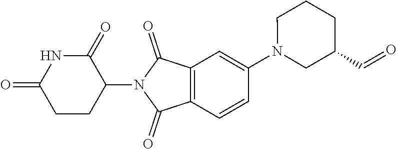 [SMILES: O=C[C@H]1CCCN(c2ccc3c(c2)C(=O)N(C2CCC(=O)NC2=O)C3=O)C1]

Step 2: Followed General Procedure B with (S)-piperidin-3-ylmethanol hydrochloride to afford 2-(2,6-dioxopiperidin-3-yl)-5-((S)-3-(hydroxymethyl)piperidin-1-yl)isoindoline-1,3-dione (493.1 mg, 73%) as a yellow solid. ¹H NMR (300 MHz, DMSO-d₆/D₂O) δ 7.65 (d, J 8.4 Hz, 1H), 7.26 (d, J 2.1 Hz, 1H), 7.19 (dd, J 8.4, 2.1 Hz, 1H), 5.04 (dd, J=12.9, 5.4 Hz, 1H), 4.00-3.90 (m, 2H), 3.38-3.32 (m, 2H), 3.13-2.71 (m, 3H), 2.67-2.44 (m, 2H), 2.03-1.98 (m, 1H), 1.76-1.67 (m, 3H), 1.57-1.38 (m, 1H), 1.34-1.10 (m, 1H). MS (ESI) calc'd for (C₁₉H₂₁N₃O₅) [M+H]⁺, 372.1; found 372.1.

Step 3: (3S)-1-(2-(2,6-dioxopiperidin-3-yl)-1,3-dioxoisoindolin-5-yl)piperidine-3-carbaldehyde LCMS C₁₉H₁₉N₃O₅ requires: 369, found: m/z=370 [M+H]⁺.

### Example 15F: Synthesis of (3R)-1-(2-(2,6-dioxopiperidin-3-yl)-1,3-dioxoisoindolin-5-yl)pyrrolidine-3-carbaldehyde

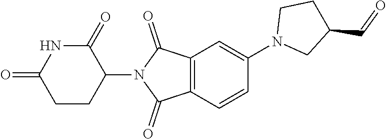 [SMILES: O=C[C@@H]1CCN(c2ccc3c(c2)C(=O)N(C2CCC(=O)NC2=O)C3=O)C1]

Step 2: Followed General Procedure B with (R)-pyrrolidin-3-ylmethanol to afford 2-(2,6-dioxopiperidin-3-yl)-5-((R)-3-(hydroxymethyl)pyrrolidin-1-yl)isoindoline-1,3-dione (480.6 mg, 74%) as a yellow solid. ¹H NMR (300 MHz, DMSO-d₆) δ 11.08 (s, 1H), 7.64 (d, J 8.4 Hz, 1H), 6.89 (d, J 2.1 Hz, 1H), 6.80 (dd, J 8.4, 2.1 Hz, 1H), 5.06 (dd, J 12.9, 5.4 Hz, 1H), 4.78 (s, 1H), 3.65-3.36 (m, 5H), 3.22-3.17 (m, 1H), 2.95-2.83 (m, 1H), 2.67-2.44 (m, 3H), 2.11-1.89 (m, 2H), 1.87-1.78 (m, 1H). MS (ESI) calc'd for (C₁₈H₁₉N₃O₅) [M+H]⁺, 358.1; found 358.1.

Step 3: (3R)-1-(2-(2,6-dioxopiperidin-3-yl)-1,3-dioxoisoindolin-5-yl)pyrrolidine-3-carbaldehyde LCMS C₁₈H₁₇N₃O₅ requires: 355, found: m/z=356 [M+H]⁺.

### Example 15G: Synthesis of (3S)-1-(2-(2,6-dioxopiperidin-3-yl)-1,3-dioxoisoindolin-5-yl)pyrrolidine-3-carbaldehyde

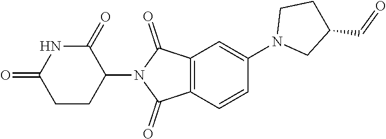 [SMILES: O=C[C@H]1CCN(c2ccc3c(c2)C(=O)N(C2CCC(=O)NC2=O)C3=O)C1]

Step 2: Followed General Procedure B with (S)-pyrrolidin-3-ylmethanol to afford 2-(2,6-dioxopiperidin-3-yl)-5-((S)-3-(hydroxymethyl)pyrrolidin-1-yl)isoindoline-1,3-dione (643.1 mg, 33%) as a yellow solid. ¹H NMR (300 MHz, DMSO-d₆) δ 11.08 (s, 1H), 7.64 (d, J 8.4 Hz, 1H), 6.89 (d, J 2.1 Hz, 1H), 6.80 (dd, J 8.4, 2.1 Hz, 1H), 5.06 (dd, J 12.9, 5.4 Hz, 1H), 4.78 (t, J 5.4 Hz, 1H), 3.59-3.41 (m, 5H), 3.22-3.17 (m, 1H), 2.95-2.83 (m, 1H), 2.67-2.44 (m, 3H), 2.12-1.88 (m, 2H), 1.87-1.76 (m, 1H). MS (ESI) calc'd for (C₁₈H₁₉N₃O₅) [M+H]⁺, 358.1; found 358.1.

Step 3: (3S)-1-(2-(2,6-dioxopiperidin-3-yl)-1,3-dioxoisoindolin-5-yl)pyrrolidine-3-carbaldehyde LCMS C₁₈H₁₇N₃O₅ requires: 355, found: m/z=356 [M+H]⁺.

### Example 15H: Synthesis of 2-(2,6-dioxopiperidin-3-yl)-5-((1R,5S,6r)-6-(hydroxymethyl)-3-aza-bicyclo[3.1.0]hexan-3-yl)isoindoline-1,3-dione

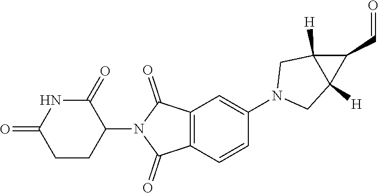 [SMILES: O=C[C@@H]1[C@H]2CN(c3ccc4c(c3)C(=O)N(C3CCC(=O)NC3=O)C4=O)C[C@@H]12]

Step 2: Followed General Procedure B with ((1R,5S,6r)-3-azabicyclo[3.1.0]hexan-6-yl)methanol to afford 2-(2,6-dioxopiperidin-3-yl)-5-((1R,5S,6r)-6-(hydroxymethyl)-3-aza-bicyclo[3.1.0]hexan-3-yl)isoindoline-1,3-dione (315.8 mg, 21%) as a yellow solid. ¹H NMR (300 MHz, DMSO-d₆) δ 11.08 (s, 1H), 7.63 (d, J 8.4 Hz, 1H), 6.92 (d, J 2.1 Hz, 1H), 6.82 (dd, J 8.4, 2.1 Hz, 1H), 5.06 (dd, J 12.6, 5.4 Hz, 1H), 4.59 (t, J 5.4 Hz, 1H), 3.64-3.60 (m, 2H), 3.50-3.35 (m, 4H), 3.00-2.76 (m, 1H), 2.58-2.44 (m, 2H), 2.07-1.91 (m, 1H), 1.69 (s, 2H), 0.86-0.79 (m, 1H). MS (ESI) calc'd for (C₁₉H₁₉N₃O₅) [M+H]⁺, 370.1; found 370.1.

Step 3: (1R,5S,6r)-3-(2-(2,6-dioxopiperidin-3-yl)-1,3-dioxoisoindolin-5-yl)-3-azabicyclo[3.1.0]hexane-6-carbaldehyde LCMS C₁₉H₁₇N₃O₅ requires: 367, found: m/z=368 [M+H]⁺.

### Example 16: General Procedure C

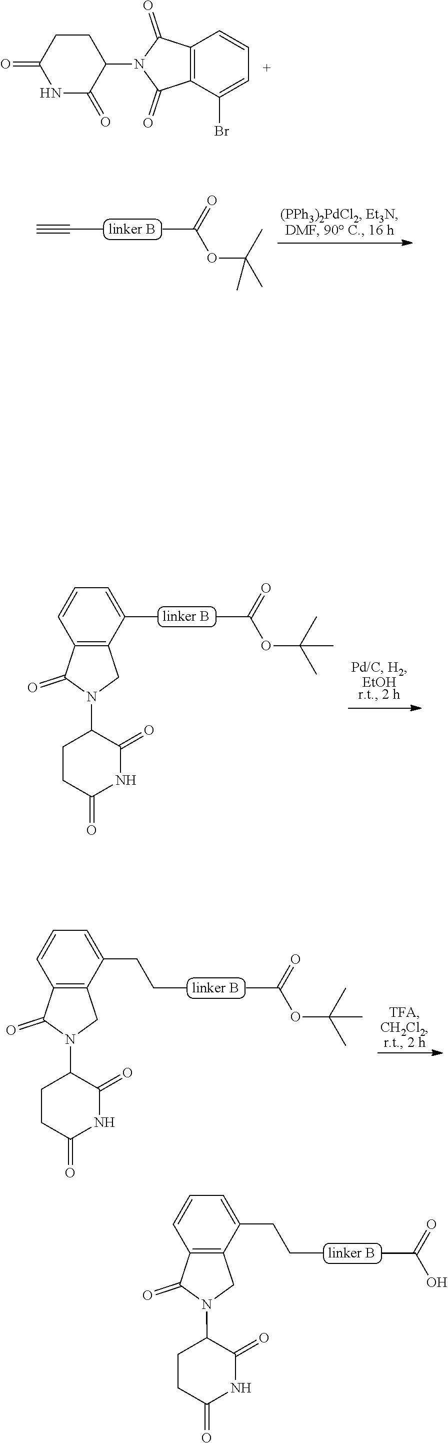

As used in General Procedure B, “linker B” is —(CH₂—CH₂—O)ₓ—, wherein x is an integer from 1 to 3.

Step 1: A mixture of 3-(4-bromo-1-oxo-2,3-dihydro-1H-isoindol-2-yl)piperidine-2,6-dione (2.52 mmol), (PPh₃)₂PdCl₂ (0.15 mmol), CuI (0.25 mmol), alkyne ester (5.04 mmol) were added to a vial. The vial was evacuated and backfilled with N₂ 5 times. DMF and triethylamine (30.3 mmol) were added and the mixture was allowed to stir at 90° C. overnight. The mixture was filtered through celite, washing with MeOH and EtOAc. EtOAc and saturated aqueous NaCl were added. The organic layer was dried with MgSO₄, filtered, concentrated and purified by reverse phase MPLC (5-100% MeCN in H₂O on C18 column) to afford the product.

Step 2: A mixture of disubstituted alkyne (0.81 mmol), Pd/C 10 wt % (0.08 mmol) and EtOH were mixed in a flask. The flask was evacuated and backfilled with H₂ 5 times and allowed to stir at r.t. for 2 h. The mixture was filtered through celite washing with MeOH and EtOAc, concentrated and carried to the next step.

Step 3: A mixture of tert-butylester (0.81 mmol), CH₂C₁₂ (2 mL), and TFA (2 mL) was allowed to stir at r.t. for 2 h. The mixture was concentrated to afford the carboxylic acid product.

### Example 16A: Synthesis of 3-(3-(2-(2,6-dioxopiperidin-3-yl)-1-oxoisoindolin-4-yl)propoxy)propanoic acid

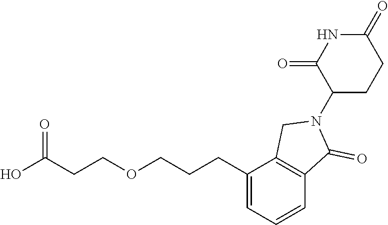 [SMILES: O=C(O)CCOCCCc1cccc2c1CN(C1CCC(=O)NC1=O)C2=O]

Step 1 product: tert-butyl 3-((3-(2-(2,6-dioxopiperidin-3-yl)-1-oxoisoindolin-4-yl)prop-2-yn-1-yl)oxy)propanoate (347 mg, 32.3%). LCMS: C₂₃H₂₆N₂O₆ requires: 426, found: m/z=427 [M+H]⁺.

Step 2 product: tert-butyl 3-(3-(2-(2,6-dioxopiperidin-3-yl)-1-oxoisoindolin-4-yl)propoxy)propanoate (350 mg, 99%). LCMS: C₂₃H₃₀N₂O₆ requires: 430, found: m/z=431 [M+H]⁺.

Step 3 product: 3-(3-(2-(2,6-dioxopiperidin-3-yl)-1-oxoisoindolin-4-yl)propoxy)propanoic acid (304 mg, 99%). LCMS: C₁₉H₂₂N₂O₆ requires: 374, found: m/z=375 [M+H]⁺.

### Example 17: Synthesis of (1s,3s)-3-(3-(2-(2,6-dioxopiperidin-3-yl)-1-oxoisoindolin-4-yl)propoxy)cyclobutane-1-carboxylic acid

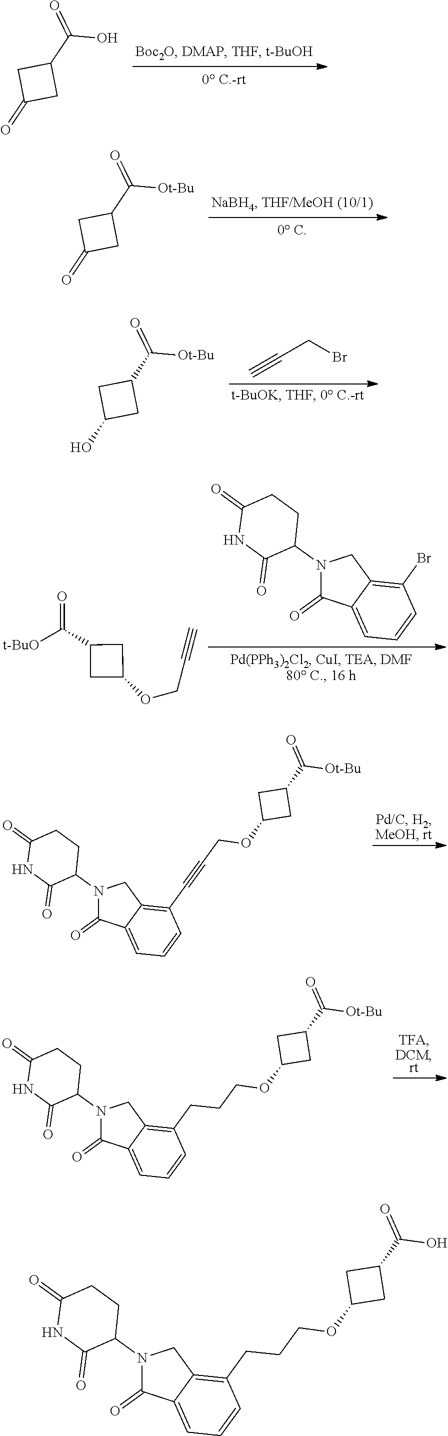

### Step 1: tert-butyl 3-oxocyclobutane-1-carboxylate

To a solution of 3-oxocyclobutane-1-carboxylic acid (5.0 g, 43.82 mmol) and DMAP (2.7 g, 21.91 mmol) in t-BuOH (20 mL) and THF (20 mL) was added a solution of Boc₂O (14.3 g, 65.73 mmol) in THF (10 mL) dropwise at 0° C. under nitrogen atmosphere. The mixture was stirred at room temperature for 16 h. The reaction was quenched by the addition of water and extracted with ethyl acetate. The combined organic layer was washed with brine, dried over anhydrous Na₂SO₄ and concentrated under vacuum. The residue was purified by flash column chromatography with 0-15% ethyl acetate in petroleum ether to afford tert-butyl 3-oxocyclobutane-1-carboxylate (6.2 g, 83%) as colorless oil. MS (ESI) calc'd for (C₉H₁₄O₃) [M+1]+, 171.1; found, 171.2. ¹H NMR (300 MHz, Chloroform-d) δ 3.39-2.98 (m, 5H), 1.44 (s, 9H).

### Step 2: tert-butyl (1s,3s)-3-hydroxycyclobutane-1-carboxylate

To a solution of tert-butyl 3-oxocyclobutane-1-carboxylate (5.1 g, 29.96 mmol) in THF (50 mL) and MeOH (5 mL) was added NaBH₄ (566.8 mg, 14.98 mmol) in portions at 0° C. under nitrogen atmosphere. The resulting mixture was stirred at 0° C. for 30 min. The reaction was then quenched by the addition of ice water and extracted with ethyl acetate. The combined organic layer was washed with brine, dried over anhydrous Na₂SO₄ and concentrated under vacuum to afford tert-butyl (1s,3s)-3-hydroxycyclobutane-1-carboxylate (4.8 g, 93%) as a light yellow oil, which was used for the next step without further purification. MS (ESI) calc'd for (C₉H₁₆O₃) [M+1]+, 173.1; found, 173.0. ¹H NMR (300 MHz, Chloroform-d) δ 4.20-4.07 (m, 1H), 2.61-2.43 (m, 3H), 2.30 (s, 1H), 2.17-2.20 (m, 2H), 1.43 (s, 9H).

### Step 3: tert-butyl (1s,3s)-3-(prop-2-yn-1-yloxy)cyclobutane-1-carboxylate

To a solution of tert-butyl (1s,3s)-3-hydroxycyclobutane-1-carboxylate (5.0 g, 29.03 mmol) and 3-bromoprop-1-yne (3.8 g, 31.94 mmol) in THE was added t-BuOK (32 mL, 1 M in THF, 32.0 mmol) dropwise at 0° C. under nitrogen atmosphere. The mixture was stirred at room temperature for 16 h. The reaction was then quenched by the addition of ice water and extracted with ethyl acetate. The combined organic layer was washed with brine, dried over anhydrous Na₂SO₄ and concentrated under vacuum. The residue was purified by flash column chromatography with 0˜20% ethyl acetate in petroleum ether to afford tert-butyl (1s,3s)-3-(prop-2-yn-1-yloxy)cyclobutane-1-carboxylate (3.2 g, 52%) as a light yellow oil. MS (ESI) calc'd for (C₁₂H₁₈O₃) [M+1]⁺, 211.1; found, 211.3. ¹H NMR (300 MHz, Chloroform-d) δ 4.17-3.99 (m, 3H), 2.63-2.43 (m, 3H), 2.29-2.11 (m, 2H), 2.04 (s, 1H), 1.44 (s, 9H).

### Step 4: tert-butyl (1s,3s)-3-((3-(2-(2,6-dioxopiperidin-3-yl)-1-oxoisoindolin-4-yl)prop-2-yn-1-yl)oxy)cyclobutane-1-carboxylate

A mixture of 3-(4-bromo-1-oxoisoindolin-2-yl)piperidine-2,6-dione (3.3 g, 10.21 mmol), tert-butyl (1s,3s)-3-(prop-2-yn-1-yloxy)cyclobutane-1-carboxylate (3.2 g, 15.32 mmol), Pd(PPh₃)₂Cl₂ (1.1 g, 1.53 mmol) and CuI (486.2 mg, 2.55 mmol) in triethylamine (30 mL) and DMF (30 mL) was stirred at 80° C. for 16 h under nitrogen atmosphere. After cooled down to room temperature, the reaction was diluted with saturated NH₄C₁ aqueous solution and then extracted with ethyl acetate. The combined organic layer was washed with brine, dried over anhydrous Na₂SO₄ and concentrated under vacuum. The residue was purified by flash column chromatography with 0-10% ethyl acetate in methanol to afford tert-butyl (1s,3s)-3-((3-(2-(2,6-dioxopiperidin-3-yl)-1-oxoisoindolin-4-yl)prop-2-yn-1-yl)oxy)cyclobutane-1-carboxylate (1.5 g, 27%) as a light yellow solid. MS (ESI) calc'd for (C₂₅H₂₈N₂O₆) [M+1]⁺, 453.2; found, 453.3.

### Step 5: tert-butyl (1s,3s)-3-(3-(2-(2,6-dioxopiperidin-3-yl)-1-oxoisoindolin-4-yl)propoxy)cyclobutane-1-carboxylate

To a solution of tert-butyl (1s,3s)-3-((3-(2-(2,6-dioxopiperidin-3-yl)-1-oxoisoindolin-4-yl)prop-2-yn-1-yl)oxy)cyclobutane-1-carboxylate (1.5 g, 2.75 mmol) in MeOH (20 mL) was added Pd/C (10%, 200 mg) under nitrogen atmosphere. The mixture was stirred at room temperature for 16 h under hydrogen atmosphere (2 atm). The solid was filtered out through a Celite pad and the filtrate was concentrated under vacuum. The residue was purified by reverse phase flash column chromatography with 10˜70% acetonitrile in water to afford tert-butyl (1s,3s)-3-(3-(2-(2,6-dioxopiperidin-3-yl)-1-oxoisoindolin-4-yl)propoxy)cyclobutane-1-carboxylate (650 mg, 43%) as a light yellow solid. MS (ESI) calc'd for (C₂₅H₃₂N₂O₆) [M+1]⁺, 457.2; found, 457.3.

### Step 6: (1s,3s)-3-(3-(2-(2,6-dioxopiperidin-3-yl)-1-oxoisoindolin-4-yl)propoxy)cyclobutane-1-carboxylic acid

A mixture of tert-butyl (1s,3s)-3-(3-(2-(2,6-dioxopiperidin-3-yl)-1-oxoisoindolin-4-yl)propoxy)cyclobutane-1-carboxylate (1.2 g, 2.63 mmol) in TFA (4 mL) and DCM (12 mL) was stirred at room temperature for 2 h before concentrated under vacuum. The residue was purified by reverse phase flash column chromatography to afford (1s,3s)-3-(3-(2-(2,6-dioxopiperidin-3-yl)-1-oxoisoindolin-4-yl)propoxy)cyclobutane-1-carboxylic acid (968.7 mg, 92%). MS (ESI) calc'd for (C₂₁H₂₄N₂O₆) [M+1]⁺, 401.2; found, 400.8. ¹H NMR (300 MHz, DMSO-d₆) δ 12.12 (s, 1H), 10.97 (s, 1H), 7.59-7.53 (m, 1H), 7.48-7.41 (m, 2H), 5.15-5.09 (m, 1H), 4.45 (d, J=17.1 Hz, 1H), 4.29 (d, J=17.1 Hz, 1H), 3.89-3.69 (m, 1H), 3.27 (t, J=6.3 Hz, 2H), 2.97-2.85 (m, 1H), 2.77-2.51 (m, 4H), 2.46-2.28 (m, 3H), 2.14-1.73 (m, 3H), 1.86-1.73 (m, 2H).

### Example 18: Synthesis of 3-(4-bromo-1-oxoisoindolin-2-yl)piperidine-2,6-dione

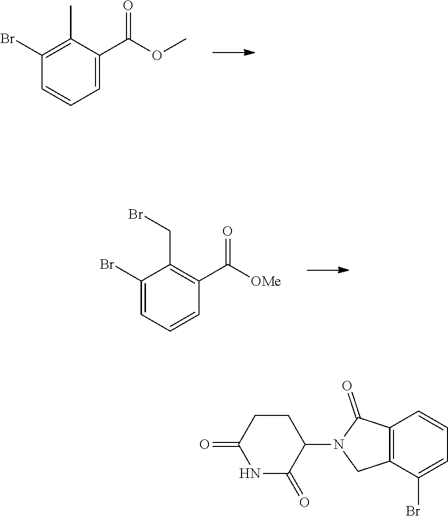 [SMILES: COC(=O)c1cccc(Br)c1C ; COC(=O)c1cccc(Br)c1CBr ; O=C1CCC(N2Cc3c(Br)cccc3C2=O)C(=O)N1]

### Step 1: methyl 3-bromo-2-(bromomethyl)benzoate

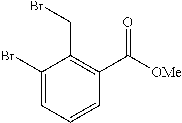 [SMILES: COC(=O)c1cccc(Br)c1CBr]

To a solution of methyl 3-bromo-2-methyl-benzoate (50 g, 218.27 mmol, 1 eq), NBS (46.62 g, 261.93 mmol, 1.2 eq) in CHCl₃ (400 mL) was added AIBN (3.58 g, 21.83 mmol, 0.1 equiv.). The mixture was stirred at 70° C. for 12 h. The reaction mixture was concentrated in vacuum, diluted with DCM (400 mL), washed with H₂O (100 mL) and brine (100 mL), extracted with DCM (100 mL), and washed with brine (50 mL) again. The organic phase was combined, dried over Na₂SO₄, and concentrated in vacuum. The residue was purified by flash silica gel chromatography (Petroleum ether/Ethyl acetate=100/1) to yield 3-bromo-2-(bromomethyl)benzoate (63 g, 204.57 mmol, 93.72% yield) as a light yellow solid.

### Step 2: 3-(4-bromo-1-oxo-isoindolin-2-yl)piperidine-2,6-dione

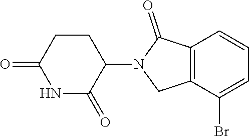 [SMILES: O=C1CCC(N2Cc3c(Br)cccc3C2=O)C(=O)N1]

To a solution of methyl 3-bromo-2-(bromomethyl)benzoate (88.2 g, 286.39 mmol, 1 eq) in ACN (600 mL) was added DIEA (49.23 g, 380.91 mmol, 66.35 mL, 1.33 equiv.) and 3-aminopiperidine-2,6-dione hydrochloride (51.01 g, 309.94 mmol, 1.08 eq). The mixture was stirred at 80° C. for 16 hr. The reaction mixture was filtered. The filter cake was triturated by a mixture solution (EtOAc: H₂O=100 mL: 200 mL) to yield 3-(4-bromo-1-oxo-isoindolin-2-yl)piperidine-2,6-dione (56.5 g, 174.85 mmol, 61.05% yield) as a purple powder.

### Example 19: Synthesis of 2-(4-(3-(2-(2,6-dioxopiperidin-3-yl)-1-oxoisoindolin-4-yl)propyl)piperazin-1-yl)acetic acid

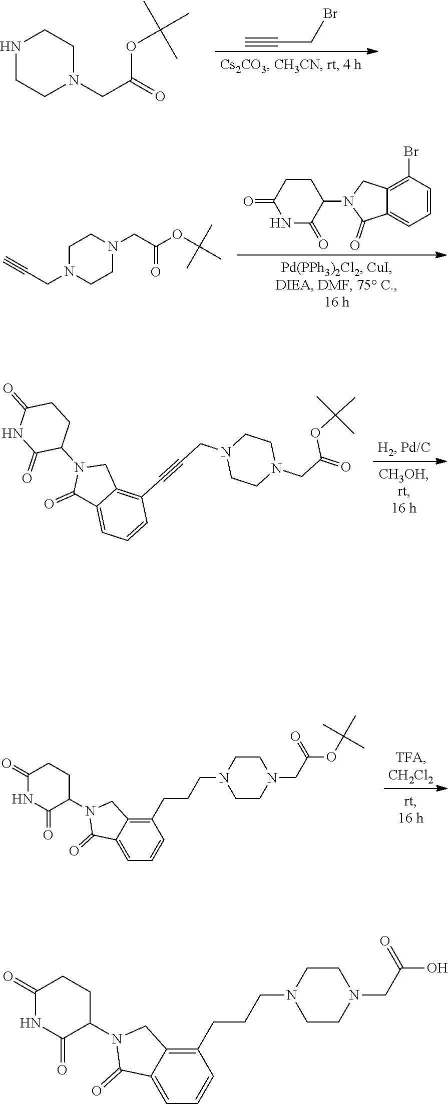

### Step 1: tert-butyl 2-(4-(prop-2-ynyl)piperazin-1-yl)acetate

To a solution of tert-butyl 2-(piperazin-1-yl)acetate (1.5 g, 7.49 mmol) in acetonitrile (50 mL) were added 3-bromoprop-1-yne (892.5 mg, 7.50 mmol) and Cs₂CO₃ (2.4 g, 7.50 mmol). The resulting solution was stirred at room temperature for 4 h. The solids were filtered out and the filtrate was evaporated under vacuum. The residue was purified by phase flash column chromatography with 0-30% ethyl acetate in petroleum ether to afford tert-butyl 2-(4-(prop-2-ynyl)piperazin-1-yl)acetate (1.1 g, 62%) as a yellow oil. MS (ESI) calculated for (C₁₃H₂₂N₂O₂) [M+H]⁺, 239.2; found, 239.1.

### Step 2: tert-butyl 2-(4-(3-(2-(2,6-dioxopiperidin-3-yl)-1-oxoisoindolin-4-yl)prop-2-ynyl)piperazin-1-yl)acetate

To a degassed solution of 3-(4-bromo-1-oxoisoindolin-2-yl)piperidine-2,6-dione (1.5 g, 4.64 mmol) in N,N-dimethylformamide (30 mL) were added tert-butyl 2-(4-(prop-2-ynyl)piperazin-1-yl)acetate (1.5 g, 6.29 mmol), Pd(PPh₃)₂Cl₂ (489.0 mg, 0.70 mmol), DIEA (20 mL) and CuI (221.7 mg, 1.16 mmol). The resulting solution was stirred at 75° C. for 16 h under nitrogen. The reaction was quenched by the addition of water, and then extracted with ethyl acetate. The combined organic layer was washed with brine, dried over anhydrous sodium sulfate and filtered. The filtrate was evaporated under vacuum. The residue was purified by flash column chromatography with 0-10% methanol in dichloromethane to afford tert-butyl 2-(4-(3-(2-(2,6-dioxopiperidin-3-yl)-1-oxoisoindolin-4-yl)prop-2-ynyl)piperazin-1-yl)acetate (1.5 g, 68%) as a yellow solid. MS (ESI) calculated for (C₂₆H₃₂N₄O₅) [M+H]⁺, 481.2; found, 481.1.

### Step 3: tert-butyl 2-(4-(3-(2-(2,6-dioxopiperidin-3-yl)-1-oxoisoindolin-4-yl)propyl)piperazin-1-yl)acetate

To a solution of tert-butyl 2-(4-(3-(2-(2,6-dioxopiperidin-3-yl)-1-oxoisoindolin-4-yl)prop-2-ynyl)piperazin-1-yl)acetate (2.2 g, 4.58 mmol) in methanol (50 mL) was added Pd/C (dry, 0.44 g). The resulting solution was stirred at room temperature for 16 h under hydrogen (2 atm). The solids were filtered out. The filtrate was evaporated under vacuum to afford tert-butyl 2-(4-(3-(2-(2,6-dioxopiperidin-3-yl)-1-oxoisoindolin-4-yl)propyl)piperazin-1-yl)acetate (1.4 g, crude) as a yellow oil, which was used in the next step without further purification. MS (ESI) calculated for (C₂₆H₃₆N₄O₅) [M+H]⁺, 485.3; found, 485.2.

### Step 4: 2-(4-(3-(2-(2,6-dioxopiperidin-3-yl)-1-oxoisoindolin-4-yl)propyl)piperazin-1-yl)acetic acid TFA salt

To a solution of tert-butyl 2-(4-(3-(2-(2,6-dioxopiperidin-3-yl)-1-oxoisoindolin-4-yl)propyl)piperazin-1-yl)acetate (1.4 g, 2.89 mmol) in dichloromethane (20 mL) was added trifluoroacetic acid (20 mL). The resulting solution was stirred at room temperature for 16 h before concentrated under vacuum. The residue was purified by HPLC (MeCN/H₂O with TFA) to afford 2-(4-(3-(2-(2,6-dioxopiperidin-3-yl)-1-oxoisoindolin-4-yl)propyl)piperazin-1-yl)acetic acid TFA salt (434.3 mg, 35%) as a yellow solid. MS (ESI) calculated for (C₂₂H₂₈N₄O₅) [M+H]⁺, 429.2; found, 429.0. ¹H NMR (300 MHz, DMSO-d₆) δ 11.08 (s, 1H), 7.60-7.65 (m, 1H), 7.52-7.47 (m, 2H), 5.20-5.13 (m, 1H), 4.52-4.46 (m, 1H), 4.35-4.29 (m, 1H), 3.51 (s, 3H), 3.47-2.84 (m, 9H), 2.72-2.50 (m, 4H), 2.49-2.31 (m, 1H), 2.05-1.97 (m, 3H).

### Example 20: Synthesis of 2-(4-(2-(2-(2,6-dioxopiperidin-3-yl)-1-oxoisoindolin-4-yl)ethyl)piperazin-1-yl)acetic acid

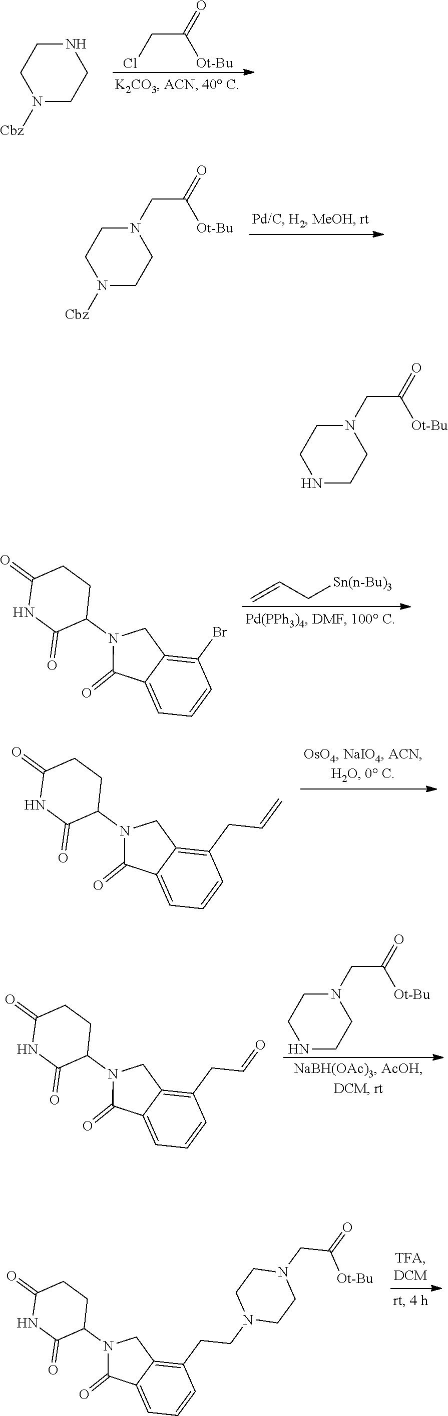

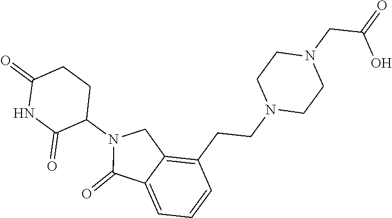 [SMILES: O=C(O)CN1CCN(CCc2cccc3c2CN(C2CCC(=O)NC2=O)C3=O)CC1]

### Step 1: benzyl 4-(2-(tert-butoxy)-2-oxoethyl)piperazine-1-carboxylate

To a solution of benzyl piperazine-1-carboxylate (10.0 g, 45.4 mmol) and K₂CO₃ (12.6 g, 90.8 mmol) in acetonitrile (150 mL) was added tert-butyl 2-chloroacetate (7.5 g, 49.9 mmol). The resulting solution was stirred at 40° C. for 16 h under nitrogen atmosphere. The solids were filtered out and the filtrate was concentrated under vacuum. The residue was purified by flash column chromatography with 0˜50% ethyl acetate in petroleum ether to afford benzyl 4-(2-(tert-butoxy)-2-oxoethyl)piperazine-1-carboxylate (9.6 g, 63%) as a light yellow oil. MS (ESI) calculated for (C₁₈H₂₆N₂O₄) [M+H]⁺, 335.2; found, 335.3.

### Step 2: tert-butyl 2-(piperazin-1-yl)acetate

To a solution of benzyl 4-(2-(tert-butoxy)-2-oxoethyl)piperazine-1-carboxylate (9.6 g, 28.7 mmol) in methanol (100 mL) was added Pd/C (10%, 2.0 g) under nitrogen atmosphere. The mixture was stirred at room temperature for 16 h under hydrogen atmosphere (2 atm). The solids were filtered out and the filtrate was concentrated under vacuum to afford tert-butyl 2-(piperazin-1-yl)acetate (6.2 g, crude) as a light yellow oil, which was used in the next step without further purification. MS (ESI) calculated for (C₁₀H₂₀N₂O₂) [M+H]⁺, 201.2; found, 201.0.

### Step 3: 3-(4-allyl-1-oxoisoindolin-2-yl)piperidine-2,6-dione

A degassed mixture of 3-(4-bromo-1-oxo-2,3-dihydro-1H-isoindol-2-yl)piperidine-2,6-dione (10.0 g, 30.9 mmol), allyltributylstannane (15.4 g, 46.4 mmol) and Pd(PPh₃)₄ (3.6 g, 3.1 mmol) in DMF (80 mL) was stirred at 100° C. for 16 h under nitrogen atmosphere. When the reaction was completed by LCMS, the resulting mixture was diluted with water and extracted with ethyl acetate. The combined organic layer was washed with brine, dried over anhydrous Na₂SO₄, filtered and concentrated under vacuum. The residue was purified by flash column chromatography with 0-10% methanol in dichloromethane to afford 3-(4-allyl-1-oxoisoindolin-2-yl)piperidine-2,6-dione (7.0 g, 79%) as a white solid. MS (ESI) calculated for (C₁₆H₁₆N₂O₃) [M+H]⁺, 285.1; found, 285.2. ¹H NMR (400 MHz, DMSO-d₆) δ 10.99 (s, 1H), 7.62-7.60 (m, 1H), 7.52-7.27 (m, 2H), 6.02-5.92 (m, 1H), 5.16-5.09 (m, 3H), 4.45 (d, J=17.2 Hz, 1H), 4.30 (d, J=17.2 Hz, 1H), 3.46-3.44 (m, 2H), 2.97-2.86 (m, 1H), 2.70-2.57 (m, 1H), 2.04-1.99 (m, 1H), 1.68-1.55 (m, 1H).

### Step 4: 2-(2-(2,6-dioxopiperidin-3-yl)-1-oxoisoindolin-4-yl)acetaldehyde

A mixture of 3-(4-allyl-1-oxoisoindolin-2-yl)piperidine-2,6-dione (7.0 g, 24.6 mmol), OsO₄ (625 mg, 2.5 mmol) and NaIO₄ (10.5 g, 49.2 mmol) in MeCN (60 mL) and H₂O (20 mL) was stirred at 0° C. for 6 h. When the reaction was completed, the resulting mixture was diluted with water and extracted with ethyl acetate. The combined organic layer was washed with brine, dried over anhydrous Na₂SO₄, filtered and concentrated under vacuum to afford 2-(2-(2,6-dioxopiperidin-3-yl)-1-oxoisoindolin-4-yl)acetaldehyde (4.0 g, crude) as a brown solid, which was used in the next step without further purification. MS (ESI) calculated for (C₁₋₅H₁₄N₂O₄) [M+H]⁺, 287.1; found, 287.2.

### Step 5: tert-butyl 2-(4-(2-(2-(2,6-dioxopiperidin-3-yl)-1-oxoisoindolin-4-yl)ethyl)piperazin-1-yl)acetate

A mixture of 2-[2-(2,6-dioxopiperidin-3-yl)-1-oxo-2,3-dihydro-1H-isoindol-4-yl]acetaldehyde (4.0 g, 13.9 mmol), tert-butyl 2-(piperazin-1-yl)acetate(3.4 g, 16.8 mmol), AcOH (1 mL) and NaBH(OAc)₃ (5.9 g, 27.9 mmol) in dichloromethane (50 mL) was stirred at room temperature for 16 h. The resulting mixture was diluted with water and extracted with ethyl acetate. The combined organic layer was washed with brine, dried over anhydrous Na₂SO₄, filtered and concentrated under vacuum. The crude residue was purified by reverse phase flash column chromatography with 10˜50% acetonitrile in water to afford tert-butyl 2-(4-(2-(2-(2,6-dioxopiperidin-3-yl)-1-oxoisoindolin-4-yl)ethyl)piperazin-1-yl)acetate (2.5 g, 22% over two steps) as a light brown syrup. MS (ESI) calculated for (C₂₅H₃₄N₄O₅) [M+H]⁺, 471.2; found, 471.0.

### Step 6: 2-(4-(2-(2-(2,6-dioxopiperidin-3-yl)-1-oxoisoindolin-4-yl)ethyl)piperazin-1-yl)acetic acid TFA salt

To a solution of tert-butyl 2-(4-(2-(2-(2,6-dioxopiperidin-3-yl)-1-oxoisoindolin-4-yl)ethyl)piperazin-1-yl)acetate (2.5 g, 5.3 mmol) in dichloromethane (20 mL) was added trifluoroacetic acid (20 mL). The resulting mixture was stirred at room temperature for 16 h before concentrated under vacuum. The residue was purified by reverse phase flash column chromatography with 5˜30% acetonitrile in water to afford 2-(4-(2-(2-(2,6-dioxopiperidin-3-yl)-1-oxoisoindolin-4-yl)ethyl)piperazin-1-yl)acetic acid (1.7214 g, 78%) as a light brown solid. MS (ESI) calculated for (C₂₁H₂₆N₄O₅) [M+H]⁺, 415.2; found, 415.4. ¹H NMR (300 MHz, DMSO-d₆) δ 11.08 (s, 1H), 7.66-7.62 (m, 1H), 7.54-7.47 (m, 2H), 5.14-5.08 (m, 1H), 4.54-4.48 (m, 1H), 4.40-4.31 (m, 1H), 3.76 (s, 2H), 3.60-3.10 (m, 10H), 3.10-2.78 (m, 3H), 2.68-2.54 (m, 1H), 2.40-2.31 (m, 1H), 2.10-1.94 (m, 1H).

### Example 21: Synthesis of 2-(4-(3-(2-(2,6-dioxopiperidin-3-yl)-1,3-dioxoisoindolin-4-yl)propyl)piperazin-1-yl)acetic acid

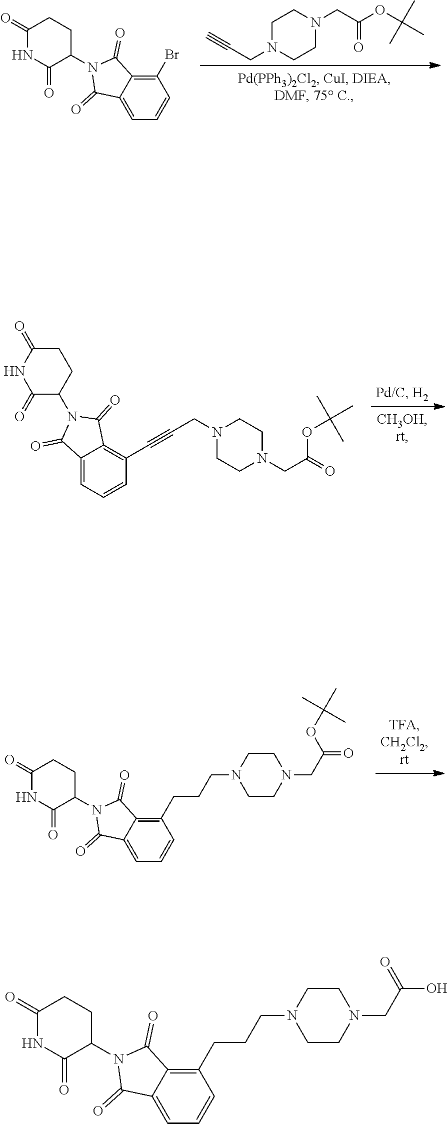 [SMILES: O=C1CCC(N2C(=O)c3cccc(Br)c3C2=O)C(=O)N1 ; C#CCN1CCN(CC(=O)OC(C)(C)C)CC1 ; CC(C)(C)OC(=O)CN1CCN(CC#Cc2cccc3c2C(=O)N(C2CCC(=O)NC2=O)C3=O)CC1 ; CC(C)(C)OC(=O)CN1CCN(CCCc2cccc3c2C(=O)N(C2CCC(=O)NC2=O)C3=O)CC1 ; O=C(O)CN1CCN(CCCc2cccc3c2C(=O)N(C2CCC(=O)NC2=O)C3=O)CC1]

### Step 1: tert-butyl 2-(4-(3-(2-(2,6-dioxopiperidin-3-yl)-1,3-dioxoisoindolin-4-yl)prop-2-yn-1-yl)piperazin-1-yl)acetate

To a degassed solution of 4-bromo-2-(2,6-dioxopiperidin-3-yl)isoindoline-1,3-dione (1.3 g, 3.86 mmol) in N,N-dimethylformamide (18 mL) were added tert-butyl 2-(4-(prop-2-ynyl)piperazin-1-yl)acetate (1.4 g, 5.57 mmol), Pd(PPh₃)₂Cl₂ (423.3 mg, 0.60 mmol), DIEA (12 mL) and CuI (251.1 mg, 1.32 mmol). The resulting solution was stirred at 75° C. for 4 h under nitrogen. The reaction was quenched by the addition of water, and then extracted with ethyl acetate. The combined organic layer was washed with brine, dried over anhydrous sodium sulfate and filtered. The filtrate was evaporated under vacuum. The residue was purified by flash column chromatography with 0˜10% methanol in dichloromethane to afford tert-butyl 2-(4-(3-(2-(2,6-dioxopiperidin-3-yl)-1,3-dioxoisoindolin-4-yl)prop-2-yn-1-yl)piperazin-1-yl)acetate (2.5 g, 70%) as a yellow solid. MS (ESI) calculated for (C₂₆H₃₀N₄O₆) [M+H]⁺, 495.2; found, 495.1.

### Step 2: tert-butyl 2-(4-(3-(2-(2,6-dioxopiperidin-3-yl)-1,3-dioxoisoindolin-4-yl)propyl)piperazin-1-yl)acetate

To a solution of tert-butyl 2-(4-(3-(2-(2,6-dioxopiperidin-3-yl)-1,3-dioxoisoindolin-4-yl)prop-2-ynyl)piperazin-1-yl)acetate (2.1 g, 4.25 mmol) in methanol (50 mL) was added Pd/C (dry, 0.42 g). The resulting solution was stirred at room temperature for 16 h under hydrogen (2 atm). The solids were filtered out and the filtrate was evaporated under vacuum to afford tert-butyl 2-(4-(3-(2-(2,6-dioxopiperidin-3-yl)-1,3-dioxoisoindolin-4-yl)propyl)piperazin-1-yl)acetate (1.6 g, crude) as a yellow solid, which was used in the next step without further purification. MS (ESI) calculated for (C₂₆H₃₄N₄O₆) [M+H]⁺, 499.2; found, 499.0.

### Step 3: 2-(4-(3-(2-(2,6-dioxopiperidin-3-yl)-1,3-dioxoisoindolin-4-yl)propyl)piperazin-1-yl)acetic acid TFA salt

To a solution of tert-butyl 2-(4-(3-(2-(2,6-dioxopiperidin-3-yl)-1,3-dioxoisoindolin-4-yl)propyl)piperazin-1-yl)acetate (2.1 g, 4.21 mmol) in dichloromethane (20 mL) was added trifluoroacetic acid (20 mL). The resulting solution was stirred at room temperature for 4 h before concentrated under vacuum. The residue was purified by Pre-HPLC with the following conditions: [Column: XSelect CSH Prep C18 OBD Column, 5 um, 19*150 mm; Mobile Phase A: Water (0.05% TFA), Mobile Phase B: ACN; Flow rate: 25 mL/min; Gradient: 5% B to 20% B in 7 min; 254/220 nm] to afford 2-(4-(3-(2-(2,6-dioxopiperidin-3-yl)-1,3-dioxoisoindolin-4-yl)propyl)piperazin-1-yl)acetic acid TFA salt (398.0 mg, 21%) as a yellow solid. MS (ESI) calculated for (C₂₂H₂₆N₄O₆) [M+H]⁺, 443.2; found, 442.9. ¹H NMR (300 MHz, DMSO-d₆) δ 11.15 (s, 1H), 7.95-7.73 (m, 3H), 5.17-5.11 (m, 1H), 3.74-3.29 (m, 3H), 3.25-2.73 (m, 11H), 2.64 (s, 1H), 2.60-2.52 (m, 1H), 2.46-2.45 (m, 1H), 2.11-1.92 (m, 3H).

### Example 22: General Procedure D

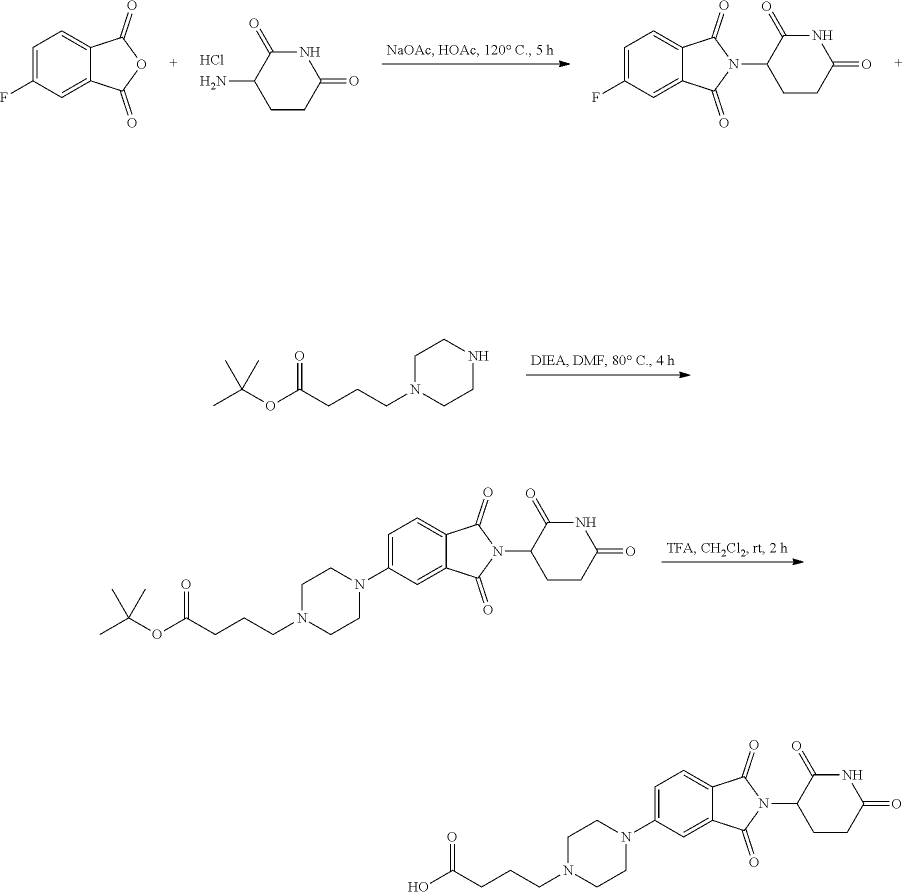

Step 1: To a solution of fluoro-benzofuran-1, 3-di one (27.16 mmol) in HOAc (50 mL) were added sodium acetate (46.17 mmol) and 3-aminopiperidine-2,6-di one hydrochloride (38.02 mmol). The reaction mixture was stirred at 120° C. for 5 h. The mixture was cooled to room temperature and diluted with water. The solids were collected by filtration and dried to afford the fluoroimide intermediate.

Step 2: To a solution of fluoroimide (0.68 mmol) in DMF (30 mL) was added tert-butyl 4-(piperazin-1-yl)butanoate (0.68 mmol) and N-ethyl-N-isopropylpropan-2-amine (1.4 mmol). The reaction mixture was stirred at 80° C. for 4 h. The resulting mixture was cooled to room temperature and diluted with water. The aqueous phase was extracted with ethyl acetate. The combined organic layer was washed with brine and water, dried over anhydrous sodium sulfate, filtered and concentrated under vacuum to afford the tert-butyl ester intermediate (3.3 g, crude) which was used in the next step without further purification.

Step 3: To a solution of the tert-butyl ester intermediate (6.57 mmol) in dichloromethane (20 mL) was added trifluoroacetic acid (10 mL). The reaction mixture was stirred at room temperature for 2 h. The solvent was removed under vacuum. The residue was purified by reverse phase flash column chromatography (20-80% acetonitrile in water) to afford the acid product (38% over 2 steps).

### Example 22A: Synthesis of 4-(4-(2-(2,6-dioxopiperidin-3-yl)-1,3-dioxoisoindolin-5-yl)piperazin-1-yl)butanoic acid

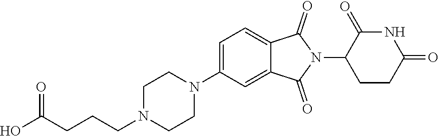 [SMILES: O=C(O)CCCN1CCN(c2ccc3c(c2)C(=O)N(C2CCC(=O)NC2=O)C3=O)CC1]

Step 1 product: 2-(2,6-dioxopiperidin-3-yl)-5-fluoroisoindoline-1,3-dione (3.0 g, 50%). LCMS: C₁₃H₉FN₂O₄ requires: 276, found: m/z=277 [M+H]⁺.

Step 2 product: tert-butyl 4-(4-(2-(2,6-dioxopiperidin-3-yl)-1,3-dioxoisoindolin-5-yl)piperazin-1-yl)butanoate (4.4 g, 84%). LCMS: C₂₅H₃₂N₄O₆ requires: 484, found: m/z=485 [M+H]⁺.

Step 3 product: 4-(4-(2-(2,6-dioxopiperidin-3-yl)-1,3-dioxoisoindolin-5-yl)piperazin-1-yl)butanoic acid TFA salt (3.35 g, 56%). LCMS: C₂₁H₂₄N₄O₆ requires: 428, found: m/z=429 [M+H]⁺.

### Example 23: General Procedure E

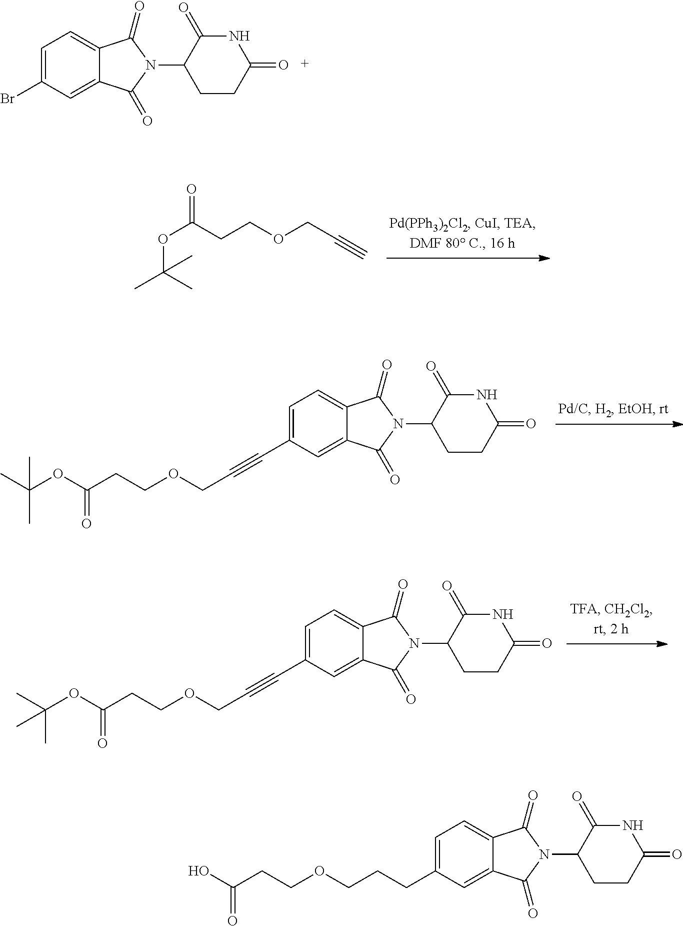 [SMILES: O=C1CCC(N2C(=O)c3ccc(Br)cc3C2=O)C(=O)N1 ; C#CCOCCC(=O)OC(C)(C)C ; CC(C)(C)OC(=O)CCOCC#Cc1ccc2c(c1)C(=O)N(C1CCC(=O)NC1=O)C2=O ; CC(C)(C)OC(=O)CCOCC#Cc1ccc2c(c1)C(=O)N(C1CCC(=O)NC1=O)C2=O ; O=C(O)CCOCCCc1ccc2c(c1)C(=O)N(C1CCC(=O)NC1=O)C2=O]

Step 1: 5-bromo-2-(2,6-dioxopiperidin-3-yl)isoindole-1,3-dione (347 mg, 1.03 mmol), (PPh₃)₃PdCl₂ (43.4 mg, 0.06 mmol), CuI (19.6 mg, 0.10 mmol) were added to a vial. The vial was evacuated and backfilled with N₂ 5 times. DMF (0.00 g, 1.03 mmol), tert-butyl 3-(prop-2-yn-1-yloxy)propanoate (190 mg, 1.03 mmol) and triethylamine (1.72 mL, 12.4 mmol) were added and the mixture was allowed to stir at 90° C. overnight. The mixture was filtered through SiO₂ washing with EtOAc/MeOH, concentrated and purified by HPLC (5-95% MeCN in H₂O with 0.1% TFA) to afford tert-butyl 3-({3-[2-(2,6-dioxopiperidin-3-yl)-1,3-dioxoisoindol-5-yl]prop-2-yn-1-yl}oxy)propanoate (173 mg, 38.2%).

Step 2: A mixture of tert-butyl 3-({3-[2-(2,6-dioxopiperidin-3-yl)-1,3-dioxoisoindol-5-yl]prop-2-yn-1-yl}oxy)propanoate (173 mg, 0.39 mmol), Pd/C 10 wt % (4.0 mg, 0.04 mmol) and EtOH (5 mL) were mixed in a flask. The flask was evacuated and backfilled with H₂ 5 times and allowed to stir at r.t. for 2 h. The mixture was filtered through celite washing with MeOH and EtOAc, concentrated and carried to the next step.

Step 3: A mixture of tert-butyl 3-{3-[2-(2,6-dioxopiperidin-3-yl)-1,3-dioxoisoindol-5-yl]propoxy}propanoate (174 mg, 0.39 mmol), CH₂C₁₂ (3 mL) and trifluoroacetic acid (1 mL) was allowed to stir at r.t. for 2 h. The volatiles were removed to afford 3-{3-[2-(2,6-dioxopiperidin-3-yl)-1,3-dioxoisoindol-5-yl]propoxy}propanoic acid (151 mg, 99.3%).

### Example No. 24: General Procedure F

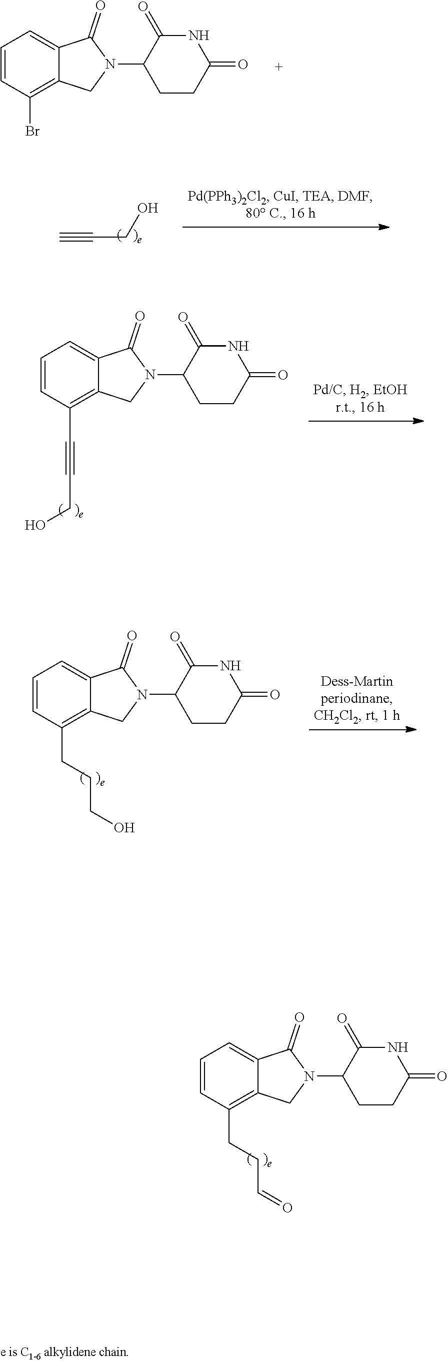

Step 1: 3-(4-bromo-1-oxo-2,3-dihydro-1H-isoindol-2-yl)piperidine-2,6-dione (1.79 mmol), (PPh₃)₂PdCl₂ (0.11 mmol), CuI (0.15 mmol) were added to a vial. The vial was evacuated and backfilled with N₂ 5 times. DMF (5 mL), alkyne (4.37 mmol) and triethylamine (18.05 mmol) were added and the mixture was allowed to stir at 90° C. overnight. The mixture was filtered through celite and purified by HPLC (5-95% MeCN in H₂O with 0.1% TFA) to afford the aryl alkyne (58%).

Step 2: A mixture of aryl alkyne (1.04 mmol), Pd/C 10 wt % (0.12 mmol) and EtOH (015 mL) were mixed in a flask. The flask was evacuated and backfilled with H₂ 5 times and allowed to stir at r.t. for 16 h. The mixture was filtered through celite washing with MeOH and EtOAc and concentrated to afford the alkyl alcohol (74%).

Step 3: Dess-Martin periodinane (1.54 mmol) was added to a mixture of the alkyl alcohol (0.77 mmol) and CH₂Cl₂ (10 mL). The mixture was allowed to stir at r.t. for 1 h. CH₂Cl₂ and aqueous Na₂SO₃ were added. The organic layer was dried with MgSO₄, filtered, concentrated and purified by MPLC (20-100% EtOAc in hexanes) to afford the aldehyde.

### Example 24A: Synthesis of 3-(2-(2,6-dioxopiperidin-3-yl)-1-oxoisoindolin-4-yl)propanal

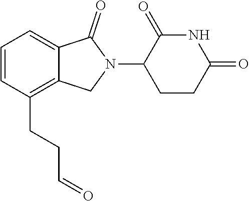 [SMILES: O=CCCc1cccc2c1CN(C1CCC(=O)NC1=O)C2=O]

Step 1 product: 3-(4-(3-hydroxyprop-1-yn-1-yl)-1-oxoisoindolin-2-yl)piperidine-2,6-dione (127.6 mg, 23.1%). LCMS; C₁₆H₁₄N₂O₄ requires: 298, found: m/z=299 [M+H]⁺.

Step 2 product: 3-(4-(3-hydroxypropyl)-1-oxoisoindolin-2-yl)piperidine-2,6-dione (129 mg, 99%). LCMS; C₁₆H₁₈N₂O₄ requires: 302, found: m/z=303 [M+H]⁺.

Step 3 product: 3-(2-(2,6-dioxopiperidin-3-yl)-1-oxoisoindolin-4-yl)propanal (29 mg, 99%). LCMS; C₁₆H₁₆N₂O₄ requires: 300, found: m/z=301 [M+H]⁺.

### Example 24B: Synthesis of 4-(2-(2,6-dioxopiperidin-3-yl)-1-oxoisoindolin-4-yl)butanal

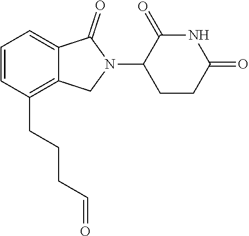 [SMILES: O=CCCCc1cccc2c1CN(C1CCC(=O)NC1=O)C2=O]

Step 1 product: 3-(4-(4-hydroxybut-1-yn-1-yl)-1-oxoisoindolin-2-yl)piperidine-2,6-dione (325 mg, 58.1%). LCMS; C₁₇H₁₆N₂O₄ requires: 312, found: m/z=313 [M+H]⁺.

Step 2 product: 3-(4-(4-hydroxybutyl)-1-oxoisoindolin-2-yl)piperidine-2,6-dione (244 mg, 74.1%). LCMS; C₁₇H₂₀N₂O₄ requires: 316, found: m/z=317 [M+H]⁺.

Step 3 product: 4-(2-(2,6-dioxopiperidin-3-yl)-1-oxoisoindolin-4-yl)butanal (178 mg, 73.4%). LCMS; C₁₇H₁₈N₂O₄ requires: 314, found: m/z=315 [M+H]⁺.

### Example 25: Synthesis of 2-(3-(2-(2,6-dioxopiperidin-3-yl)-1,3-dioxoisoindolin-4-yl)propoxy)acetaldehyde

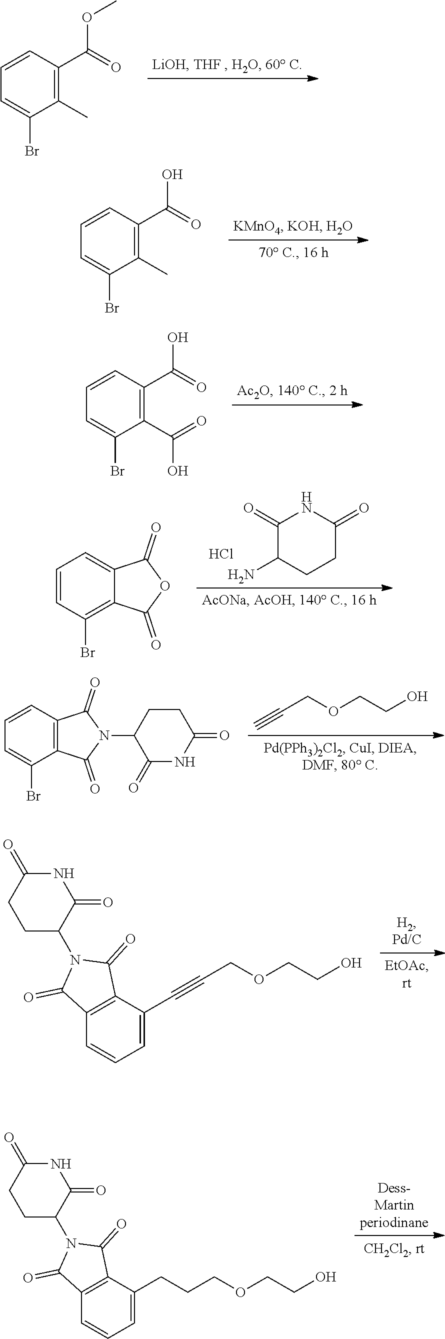

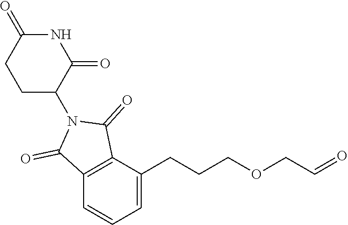 [SMILES: O=CCOCCCc1cccc2c1C(=O)N(C1CCC(=O)NC1=O)C2=O]

### Step 1: 3-bromo-2-methylbenzoic acid

A mixture of methyl 3-bromo-2-methylbenzoate (35.0 g, 152.79 mmol) and LiOH (10.9 g, 453.79 mmol) in THF (300 mL) and H₂O (50 mL) was stirred at 60° C. for 16 h and then concentrated under vacuum. The residue was diluted with water (80 mL) and the mixture was then acidified to pH 4 with 2N HCl. The precipitated solids were collected by filtration and washed with water. The solids were dried under vacuum to afford 3-bromo-2-methylbenzoic acid (30 g, 91%) as a white solid. MS (ESI) calculated for (C₈H₇BrO₂) [M+H]⁺, 214.9, 216.9; found, 215.0, 217.0.

### Step 2: 3-bromophthalic acid

To a solution of KOH (78.3 g, 1395.58 mmol) in H₂O (2.5 L) was added 3-bromo-2-methylbenzoic acid (50.0 g, 232.51 mmol) at room temperature. The mixture was stirred for 5 min and then to the mixture was added KMnO₄ (73.5 g, 465.02 mmol). The resulting mixture was stirred at 70° C. for 16 h. The mixture was cooled to room temperature and then diluted with ethanol (1.0 L). The resulting mixture was stirred for another 30 min before filtration. The filtrate was acidified to pH 4 with HCl (3 N) and extracted with ethyl acetate (1 L×3). The combined organic layers were washed with brine, dried over anhydrous Na₂SO₄, filtered and concentrated under vacuum to afford 3-bromophthalic acid (55 g, 96%) as an off-white solid. MS (ESI) calculated for (C₈H₅BrO₄) [M+H]⁺, 244.9, 246.9; found, 245.1, 247.1.

### Step 3: 4-bromoisobenzofuran-1,3-dione

A mixture of 3-bromophthalic acid (55.0 g, crude) in Ac₂O (500 mL) was stirred at 140° C. for 2 h before concentrated under vacuum. The residue was purified by trituration with ethyl acetate/petroleum ether (1/5) to afford 4-bromoisobenzofuran-1,3-dione (45 g, crude) as a light yellow solid. MS (ESI) calculated for (C₈H₃BrO₃) [M+H]⁺, 226.9, 228.9; found, 227.1, 229.1.

### Step 4: tert-butyl 3-bromo-5-methyl-1H-pyrazolo[4,3-b]pyridine-1-carboxylate

A mixture of 4-bromoisobenzofuran-1,3-dione (15.0 g, 66.08 mmol), 3-aminopiperidine-2,6-dione hydrochloride (15.2 g, 92.50 mmol) and NaOAc (9.2 g, 112.33 mmol) in AcOH (200 mL) was stirred at 140° C. for 8 h under nitrogen atmosphere. The mixture was cooled to room temperature. The solids were collected by filtration and then washed with water and ethyl acetate. The solids was dried under vacuum to afford 4-bromo-2-(2,6-dioxopiperidin-3-yl)isoindoline-1,3-dione (20 g, 89%) as an off-white solid. MS (ESI) calculated for (C₁₃H₉BrN₂O₄) [M+H]⁺, 336.9, 338.9.

### Step 5: 2-(2,6-dioxopiperidin-3-yl)-4-(3-(2-hydroxyethoxy)prop-1-yn-1-yl)isoindoline-1,3-dione

To a degassed solution of 4-bromo-2-(2,6-dioxopiperidin-3-yl)isoindoline-1,3-dione (10.0 g, 29.66 mmol) in dry N,N-dimethylformamide (160 mL) were added [1,1′-Bis(diphenylphosphino)ferrocene]dichloropalladium (II) dichloromethane adduct (3.1 g, 4.44 mmol), copper(I) iodide (1.4 g, 7.36 mmol), N-ethyl-N-isopropylpropan-2-amine (100 mL) and 2-(prop-2-yn-1-yloxy)ethan-1-ol (4.4 g, 44.34 mmol). The resulting mixture was stirred at 80° C. for 16 h under nitrogen. The reaction mixture was diluted with water and extracted with ethyl acetate. The combined organic layer was washed with water and brine, dried over anhydrous sodium sulfate, filtered and concentrated under vacuum. The residue was purified by flash column chromatography with 0-100% ethyl acetate in petroleum ether to afford 2-(2,6-dioxopiperidin-3-yl)-4-(3-(2-hydroxyethoxy)prop-1-yn-1-yl)isoindoline-1,3-dione (3.0 g, 28%) as a gray solid. MS (ESI) calc'd for (C₁₈H₁₆N₂O₆) [M+H]⁺, 357.1; found, 357.0.

### Step 6: 2-(2,6-dioxopiperidin-3-yl)-4-(3-(2-hydroxyethoxy)propyl)isoindoline-1,3-dione

A mixture of 2-(2,6-dioxopiperidin-3-yl)-4-(3-(2-hydroxyethoxy)prop-1-yn-1-yl)isoindoline-1,3-dione (2.8 g, 7.87 mmol) and Palladium/C (0.7 g, 10%) in ethyl acetate (50 mL) was stirred at room temperature for 16 h under H₂. The solids were filtered. The filtrate was concentrated under vacuum to afford the crude product. The residue was purified by reversed phase flash column chromatography with 5˜50% acetonitrile in water to afford 2-(2,6-dioxopiperidin-3-yl)-4-(3-(2-hydroxyethoxy)propyl)isoindoline-1,3-dione (882.2 mg, 31%) as a white solid. MS (ESI) calc'd for (C₁₈H₂₀N₂O₆) [M+H]⁺361.1; found, 361.1.

### Step 7: 2-(3-(2-(2,6-dioxopiperidin-3-yl)-1,3-dioxoisoindolin-4-yl)propoxy)acetaldehyde

1,1-bis(acetyloxy)-3-oxo-3H-1lambda5,2-benziodaoxol-1-yl acetate (90 mg, 0.21 mmol) was added to a mixture of 3-[4-(3-hydroxypropyl)-1-oxo-2,3-dihydro-1H-isoindol-2-yl]piperidine-2,6-dione (32 mg, 0.11 mmol) and CH₂C₁₂ (1 mL). The mixture was allowed to stir at r.t. for 1 h. The mixture was purified by MPLC (10-100% EtOAc in hexanes) to afford 2-(3-(2-(2,6-dioxopiperidin-3-yl)-1,3-dioxoisoindolin-4-yl)propoxy)acetaldehyde (35 mg, 97%).

### Example 26: Synthesis of 4-(5-(2-(2,6-dioxopiperidin-3-yl)-1-oxoisoindolin-4-ylamino)-1,3,4-oxadiazol-2-yl)butanoic acid

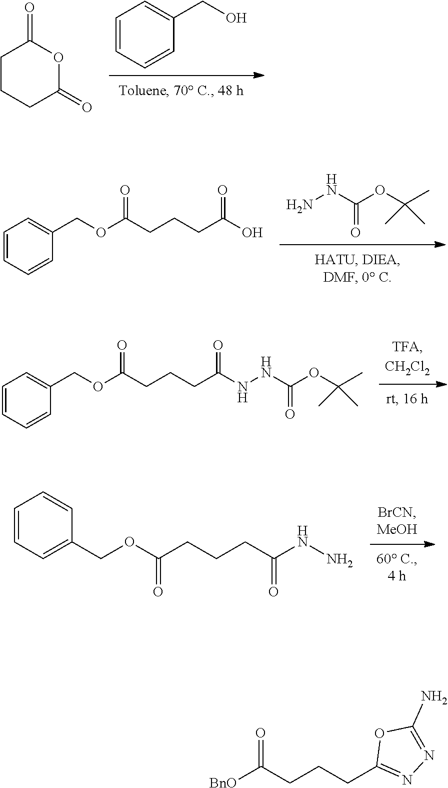

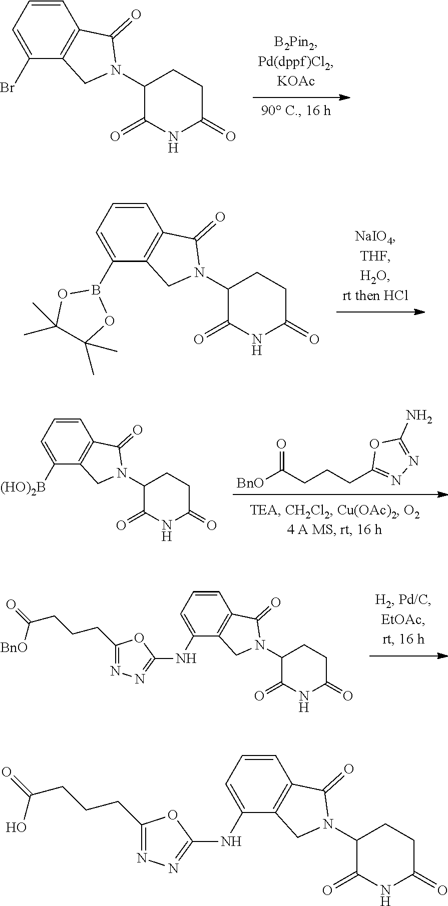

### Step 1: 5-(benzyloxy)-5-oxopentanoic acid

To a solution of dihydro-3H-pyran-2,6-dione (50.0 g, 438.21 mmol) in toluene (500 mL) was added phenylmethanol (52.1 g, 482.40 mmol). The resulting solution was stirred at 70° C. for 48 h. After the reaction was completed, the resulting mixture was concentrated under vacuum to afford 5-(benzyloxy)-5-oxopentanoic acid (90 g, crude) as colorless oil, which was used for the next step without further purification. MS (ESI) calculated for (C₁₂H₁₄O₄) [M+H]⁺, 223.1; found, 223.0.

### Step 2: tert-butyl 2-(5-(benzyloxy)-5-oxopentanoyl)hydrazinecarboxylate

To a solution of 5-(benzyloxy)-5-oxopentanoic acid (20.0 g, 89.99 mmol) in DMF (500 mL) were added tert-butyl hydrazinecarboxylate (11.9 g, 89.99 mmol), DIEA (58.1 g, 449.96 mmol) and HATU (68.4 g, 179.99 mmol). The mixture was stirred at 0° C. for 1.5 h. After the reaction was completed, the resulting mixture was diluted with ethyl acetate. The organic phase was washed with water and brine, dried over anhydrous sodium sulfate and filtered. The filtrate was concentrated under vacuum. The residue was purified by silica gel flash column chromatography with 0-50% ethyl acetate in petroleum ether to afford tert-butyl 2-(5-(benzyloxy)-5-oxopentanoyl)hydrazinecarboxylate (28.0 g, 92%) as yellow oil. MS (ESI) calculated for (C₁₇H₂₄N₂O₅) [M+H]⁺, 337.2; found [M+Na]⁺, 359.2.

### Step 3: benzyl 5-hydrazinyl-5-oxopentanoate

To a solution of tert-butyl 2-(5-(benzyloxy)-5-oxopentanoyl)hydrazinecarboxylate (18.0 g, 54.05 mmol) in CH₂C₁₂ (100 mL) was added TFA (50 mL). The mixture was stirred at room temperature for 16 h. After the reaction was completed, the reaction solution was concentrated under vacuum. The residue was dissolved in saturated NaHCO₃ aqueous solution and extracted with dichloromethane. The combined organic phase was washed with water and brine, dried over anhydrous sodium sulfate and filtered. The filtrate was concentrated under vacuum to afford benzyl 5-hydrazinyl-5-oxopentanoate (12.0 g, crude) as yellow oil, which was used for the next step without further purification. MS (ESI) calculated for (C₁₂H₁₆N₂O₃) [M+H]⁺, 237.1; found, 237.1.

### Step 4: benzyl 4-(5-amino-1,3,4-oxadiazol-2-yl)butanoate

To a solution of benzyl 5-hydrazinyl-5-oxopentanoate (13.5 g, 57.14 mmol) in MeOH (200 mL) was added carbononitridic bromide (7.3 g, 68.56 mmol). The mixture was stirred at 60° C. for 4 h. After the reaction was completed, the mixture was concentrated under vacuum. The residue was dissolved in saturated NaHCO₃ aqueous solution and extracted with dichloromethane. The combined organic phase was washed with water and brine, dried over anhydrous sodium sulfate and filtered. The filtrate was concentrated under vacuum. The residue was purified by silica gel flash column chromatography with 0-100% ethyl acetate in petroleum ether to afford benzyl 4-(5-amino-1,3,4-oxadiazol-2-yl)butanoate (8.0 g, 53%) as a white solid. MS (ESI) calculated for (C₁₃H₁₅N₃O₃) [M+H]⁺, 262.1; found, 262.1.

### Step 5: 3-(1-oxo-4-(4,4,5,5-tetramethyl-1,3,2-dioxaborolan-2-yl)isoindolin-2-yl)piperidine-2,6-dione

To a degassed solution of 3-(4-bromo-1-oxoisoindolin-2-yl)piperidine-2,6-dione (1.0 g, 3.09 mmol) in dioxane (10 mL) were added 4,4,5,5-tetramethyl-2-(4,4,5,5-tetramethyl-1,3,2-dioxaborolan-2-yl)-1,3,2-dioxaborolane (1.57 g, 6.19 mmol), Pd(dppf)C₁₂ (226 mg, 0.31 mmol) and KOAc (607 mg, 6.19 mmol). The mixture was stirred at 90° C. for 16 h under nitrogen. After the reaction was completed, the solid was filtered. The filtrate was concentrated under vacuum. The residue was purified by silica gel flash column chromatography with 0-100% ethyl acetate in petroleum ether to afford 3-(1-oxo-4-(4,4,5,5-tetramethyl-1,3,2-dioxaborolan-2-yl)-isoindolin-2-yl)piperidine-2,6-dione(1.2 g, 83%) as a yellow solid. MS (ESI) calculated for (C₁₉H₂₃BN₂O₅) [M+H]⁺, 370.2; found, 370.1.

### Step 6: 2-(2,6-dioxopiperidin-3-yl)-1-oxoisoindolin-4-ylboronic acid

To a solution of 3-[1-oxo-4-(4,4,5,5-tetramethyl-1,3,2-dioxaborolan-2-yl)-2,3-dihydro-1H-isoindol-2-yl]piperidine-2,6-dione (1.0 g, 2.75 mmol) in THF (48 mL) and H₂O (12 mL) was added NaIO₄ (2.1 g, 10.00 mmol). The mixture was stirred at room temperature for 30 min. Then 1N HCl (1.9 mL, 1.90 mmol) was added to the above mixture and stirred at room temperature for another 4 h. After the reaction was completed, the solid was filtered. The filtrate was concentrated under vacuum. The residue was purified by reverse phase FC with 5˜60% MeCN in H₂O to afford 2-(2,6-dioxopiperidin-3-yl)-1-oxoisoindolin-4-ylboronic acid (410 mg, 51%) as an off-white solid. MS (ESI) calculated for (C₁₃H₁₃BN₂O₅) [M+H]⁺, 289.1; found, 289.1

### Step 7: benzyl 4-(5-(2-(2,6-dioxopiperidin-3-yl)-1-oxoisoindolin-4-ylamino)-1,3,4-oxadi-azol-2-yl)butanoate

To a solution of 2-(2,6-dioxopiperidin-3-yl)-1-oxoisoindolin-4-ylboronic acid (310 mg, 1.07 mmol) in CH₂C₁₂ (6 mL) were added benzyl 4-(5-amino-1,3,4-oxadiazol-2-yl)butanoate (525 mg, 2.01 mmol), Cu(OAc)₂ (224 mg, 1.24 mmol), TEA (1.5 mL) and 4A MS (100 mg). The mixture was stirred at room temperature for 16 h under oxygen. After the reaction was completed, the solid was filtered. The filtrate was concentrated under vacuum. The residue was purified by reverse phase FC with 5˜65% MeCN in H₂O to afford benzyl 4-(5-(2-(2,6-dioxopiperidin-3-yl)-1-oxoisoindolin-4-ylamino)-1,3,4-oxadiazol-2-yl)butanoate (370 mg, 68%) as a yellow solid. MS (ESI) calculated for (C₂₆H₂₅N₅O₆) [M+H]⁺, 504.2; found, 504.4.

### Step 8: 4-(5-(2-(2,6-dioxopiperidin-3-yl)-1-oxoisoindolin-4-ylamino)-1,3,4-oxadiazol-2-yl)-butanoic acid

To a solution of benzyl 4-(5-(2-(2,6-dioxopiperidin-3-yl)-1-oxoisoindolin-4-ylamino)-1,3,4-oxadiazol-2-yl)butanoate (360 mg, 0.71 mmol) in ethyl acetate (5 mL) was added Pd/C (dry, 50 mg). The mixture was stirred at room temperature for 16 h under hydrogen. After the reaction was completed, the solid was filtered out. The filtrate was concentrated under vacuum. The residue was purified by reverse phase FC with 5˜55% MeCN in H₂O and then further purified by prep-HPLC with the following conditions: [Column: Sunfire prep C18 column 30*150, Sum; Mobile Phase A: Mobile Phase B: ACN; Flow rate: 60 mL/min; Gradient: 10% B to 26% B in 7 min; 254 nm] to afford 4-(5-(2-(2,6-dioxopiperidin-3-yl)-1-oxoisoindolin-4-ylamino)-1,3,4-oxadiazol-2-yl)-butanoic acid (25 mg, 8%) as a white solid. MS (ESI) calculated for (C₁₉H₁₉N₅O₆) [M+H]⁺, 414.1; found, 414.4. ¹H NMR (400 MHz, DMSO-d₆) δ 12.17 (s, 1H), 11.04 (s, 1H), 10.23 (s, 1H), 8.17-8.13 (m, 1H), 7.54 (t, J=7.6 Hz, 1H), 7.42 (d, J=7.6 Hz, 1H), 5.17-5.12 (m, 1H), 4.63-4.28 (m, 2H), 2.97-2.91 (m, 1H), 2.84-2.79 (m, 2H), 2.66-2.59 (m, 1H), 2.41-2.24 (m, 3H), 2.13-2.00 (m, 1H), 1.96-1.85 (m, 2H).

### Example 27: General Procedure G

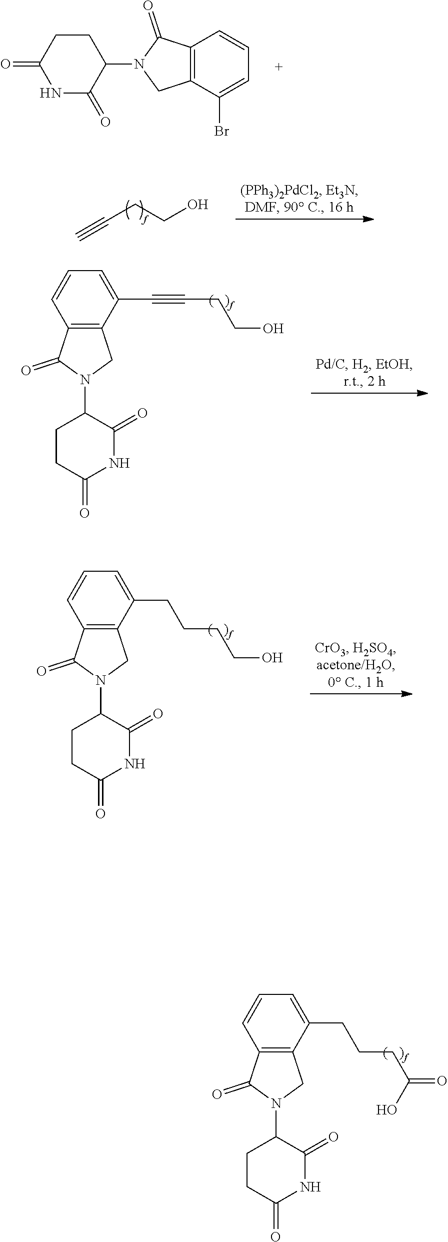

f is C₁₋₅ alkylidene chain.

Step 1: 3-(4-Bromo-1-oxoisoindolin-2-yl)piperidine-2,6-dione (4.6 mmol), copper iodide (177 mg) and bis-triphenylphosphine-palladium dichloride (326 mg) were evacuated and flushed with nitrogen 3 times. DMF (5 mL), triethylamine (6.5 mL) and the alkyne (27.9 mmol) were added and the vial was flushed with nitrogen, sealed and heated to 80° C. for 20 h. The mixture was cooled to room temperature and was diluted with DCM/ethyl acetate (1:1, 20 mL) and the solid was filtered over a pad of Celite. The solid was stirred with acetonitrile for 16 h. The solids were filtered and concentrated to give the disubstituted alkyne product.

Step 2: The disubstituted alkyne (2.2 mmol) was dissolved in methanol (40 mL). Palladium over charcoal (10%, 235 mg) was added and the flask was filled with hydrogen at 65 psi for 3 h. The mixture was filtered over Celite and washed with methanol to give the alcohol product.

Step 3: Chromic acid (360 mg, 3.6 mmol) was added to 3 M sulfuric acid (3 mL) to make a solution of chromium oxidant (Jones' reagent). The alcohol (1.2 mmol) was suspend in acetone (2.5 mL) and 3 M sulfuric acid (0.5 mL) and the suspension was cooled to 0° C. The Jones' reagent was slowly added to the alcohol suspension and allowed to stir for 1 h. The mixture was poured into of iced water (20 mL) and the solid was filtered and washed with water. The aqueous solution was extracted with (2×20 mL) EtOAc, washed with brine and concentrated. The organic fractions were combined with the solid and the mixture was purified by flash column chromatography (0-25% methanol in DCM) to afford the acid product.

### Example 27A: Synthesis of 5-(2-(2,6-dioxopiperidin-3-yl)-1-oxoisoindolin-4-yl)pentanoic acid

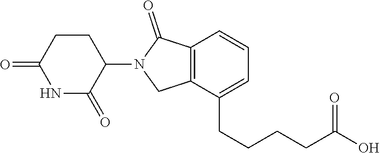 [SMILES: O=C(O)CCCCc1cccc2c1CN(C1CCC(=O)NC1=O)C2=O]

Step 1 product: 3-(4-(5-hydroxypent-1-yn-1-yl)-1-oxoisoindolin-2-yl)piperidine-2,6-dione (325 mg, 58%). LCMS; C₁₈H₁₈N₂O₄ requires: 326, found: m/z=349 [M+Na]⁺.

Step 2 product: 3-(4-(5-hydroxypentyl)-1-oxoisoindolin-2-yl)piperidine-2,6-dione (160 mg, 99%). LCMS; C₁₈H₂₂N₂O₄ requires: 330, found: m/z=353 [M+Na]⁺.

Step 3 product: 5-(2-(2,6-dioxopiperidin-3-yl)-1-oxoisoindolin-4-yl)pentanoic acid (74 mg, 60%). LCMS; C₁₈H₂₀N₂O₅ requires: 344, found: m/z=367 [M+Na]⁺.

### Example 28: Synthesis of 3-(3-(2-(2,6-dioxopiperidin-3-yl)-3-oxoisoindolin-5-yl)propoxy)propanoic acid

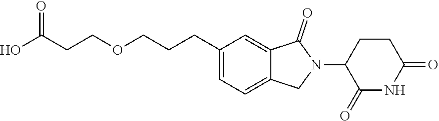 [SMILES: O=C(O)CCOCCCc1ccc2c(c1)C(=O)N(C1CCC(=O)NC1=O)C2]

### Step 1: methyl 5-bromo-2-(bromomethyl)benzoate

To a solution of methyl 5-bromo-2-methylbenzoate (24.5 g, 107.4 mmol) in CCl₄ (300 mL) were added NBS (17.1 g, 96.7 mmol) and BPO (4.8 g, 19.8 mmol). The mixture was stirred at 80° C. for 16 h under N₂. The resulting mixture was cooled down to room temperature and then filtered. The filtrate was concentrated under vacuum and the residue was purified by silica gel flash column chromatography with 0˜5% ethyl acetate in petroleum ether to afford methyl 5-bromo-2-(bromomethyl)benzoate (23.5 g, 76%) as yellow oil. 1H NMR (300 MHz, DMSO-d₆) δ 7.98 (d, J=2.1 Hz, 1H), 7.81 (dd, J=8.4, 2.1 Hz, 1H), 7.56 (d, J=8.4 Hz, 1H), 4.98 (s, 2H), 3.88 (s, 3H).

### Step 2: 3-(6-bromo-1-oxoisoindolin-2-yl)piperidine-2,6-dione

To a mixture of methyl 5-bromo-2-(bromomethyl)benzoate (23.5 g, 76.8 mmol) in MeCN (250 mL) were added 3-aminopiperidine-2,6-dione hydrochloride (19.0 g, 115.8 mmol) and TEA (31.0 g, 306.9 mmol). The mixture was stirred at 80° C. for 16 h. The resulting mixture was cooled down to room temperature and then filtered. The filtrate was concentrated under vacuum and the crude residue was purified by trituration with methanol and acetonitrile to afford 3-(6-bromo-1-oxoisoindolin-2-yl)piperidine-2,6-dione (5.8 g, 23%) as a dark blue solid. ¹H NMR (300 MHz, DMSO-d₆) δ 11.01 (s, 1H), 7.91-7.78 (m, 2H), 7.61 (d, J=8.1 Hz, 1H), 5.13 (dd, J=13.2, 5.1 Hz, 1H), 4.46 (d, J=17.7 Hz, 1H), 4.32 (d, J=17.7 Hz, 1H), 2.98-2.86 (m, 1H), 2.67-2.54 (m, 1H), 2.47-2.33 (m, 1H), 2.08-1.99 (m, 1H). MS (ESI) calc'd for (C₁₃H₁₁BrN₂O₃) [M+H]⁺, 323.0/325.0; found 322.9/324.9.

### Step 3: tert-butyl 3-((3-(2-(2,6-dioxopiperidin-3-yl)-3-oxoisoindolin-5-yl)prop-2-yn-1-yl)oxy)propanoate

3-(6-bromo-1-oxo-3H-isoindol-2-yl)piperidine-2,6-dione (511 mg, 1.58 mmol), (PPh₃)₂PdCl₂ (66.6 mg, 0.09 mmol), CuI (30.1 mg, 0.16 mmol) were added to a vial. The vial was evacuated and backfilled with N₂ 5 times. DMF (5 mL), tert-butyl 3-(prop-2-yn-1-yloxy)propanoate (437 mg, 2.37 mmol) and triethylamine (2.64 mL, 19.0 mmol) were added and the mixture was allowed to stir at 90° C. overnight. The mixture was filtered through celite washing with MeOH and EtOAc. The volatiles were removed under vacuum. EtOAc and H₂O were added. The organic layer as washed with brine, dried with MgSO₄, filtered, concentrated and purified by MPLC (0-10% MeOH in CH₂Cl₂) to afford tert-butyl 3-({3-[2-(2,6-dioxopiperidin-3-yl)-3-oxo-1H-isoindol-5-yl]prop-2-yn-1-yl}oxy)propanoate (107 mg, 15.9%). LCMS; C₂₃H₂₆N₂O₆ requires: 426, found: m/z=427 [M+H]⁺.

### Step 4: tert-butyl 3-(3-(2-(2,6-dioxopiperidin-3-yl)-3-oxoisoindolin-5-yl)propoxy)propanoate

A mixture of tert-butyl 3-({3-[2-(2,6-dioxopiperidin-3-yl)-3-oxo-1H-isoindol-5-yl]prop-2-yn-1-yl}oxy)propanoate (107 mg, 0.25 mmol), Pd/C 10 wt % (2.5 mg, 0.03 mmol) and EtOH (4 mL) were mixed in a flask. The flask was evacuated and backfilled with H₂ 5 times and allowed to stir at r.t. for 2 h. The mixture was filtered through celite washing with MeOH and EtOAc, concentrated and carried to the next step. LCMS; C₂₃H₃₀N₂O₆ requires: 430, found: m/z=431 [M+H]⁺.

### Step 5: 3-(3-(2-(2,6-dioxopiperidin-3-yl)-3-oxoisoindolin-5-yl)propoxy)propanoic acid

A mixture of tert-butyl 3-{3-[2-(2,6-dioxopiperidin-3-yl)-3-oxo-1H-isoindol-5-yl]propoxy}propanoate (106 mg, 0.25 mmol), CH₂C₁₂ (2 mL) and trifluoroacetic acid (0.4 mL) was allowed to stir at r.t. for 2 h. The volatiles were removed to afford 3-{3-[2-(2,6-dioxopiperidin-3-yl)-3-oxo-1H-isoindol-5-yl]propoxy}propanoic acid (60 mg, 65.1% over 2 steps). LCMS; C₁₉H₂₂N₂O₆ requires: 374, found: m/z=375 [M+H]⁺.

### Example 29: Synthesis of 4-((2-(2-(2-(2-aminoethoxy)ethoxy)ethoxy)ethyl)amino)-2-(2,6-dioxopiperidin-3-yl)isoindoline-1,3-dione

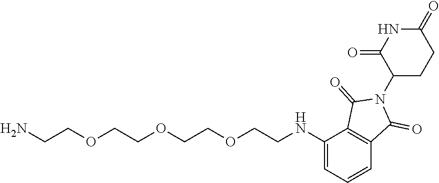 [SMILES: NCCOCCOCCOCCNc1cccc2c1C(=O)N(C1CCC(=O)NC1=O)C2=O]

Step 1: A solution of tert-butyl N-[2-[2-[2-(2-aminoethoxy)ethoxy]ethoxy]ethyl]carbamate (3 g, 10.26 mmol, 1 eq), i-Pr₂NEt (2.65 g, 20.52 mmol, 3.57 mL, 2 eq) and 2-(2,6-dioxo-3-piperidyl)-4-fluoro-isoindoline-1,3-dione (2.89 g, 10.26 mmol, 1 eq) in DMSO (40 mL) was stirred at 90° C. for 6 h. The reaction mixture was diluted with H₂O (60 mL) and extracted with EtOAc. The combined organic layer was washed with brine, dried over Na₂SO₄, and concentrated under reduced pressure. The residue was purified by reverse MPLC column (0.1% FA in H₂O). tert-butyl N-[2-[2-[2-[2-[[2-(2,6-dioxo-3-piperidyl)-1,3-dioxo-isoindolin-4-yl]amino]ethoxy]ethoxy]ethoxy]ethyl]carbamate (2.9 g, 5.29 mmol, 51.5%) was obtained as a blue oil. LCMS: C₂₆H₃₆N₄O₉ requires: 548, found: m/z=549 [M+H]⁺.

Step 2: A solution of tert-butyl N-[2-[2-[2-[2-[[2-(2,6-dioxo-3-piperidyl)-1,3-dioxo-isoindolin-4-yl]amino]ethoxy]ethoxy]ethoxy]ethyl]carbamate (2.9 g, 5.29 mmol, 1 equiv.) and HCl (4 M in dioxane, 30 mL, 22.7 equiv.) was stirred at 25° C. for 2 h under N₂. The reaction mixture was concentrated under reduced pressure. The residue was purified by HPLC (1-30% MeCN in H₂O with 0.05% HCl). 4-[2-[2-[2-(2-aminoethoxy)ethoxy]ethoxy]ethylamino]-2-(2,6-dioxo-3-piperidyl)isoindoline-1,3-dione (1.1 g, 2.07 mmol, 39.1%, 2HCl) was obtained as a yellow solid. LCMS: C₂₁H₂₈N₄O₇ requires: 448, found: m/z=449 [M+H]⁺.

### Example 30: Synthesis of 3-[4-[3-[2-[2-(2-aminoethoxy)ethoxy]ethoxy]propyl]-1-oxo-isoindolin-2-yl]piperidine-2,6-dione

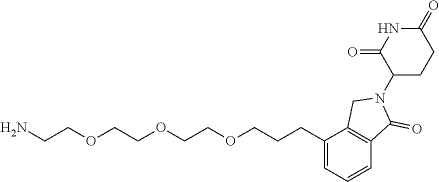 [SMILES: NCCOCCOCCOCCCc1cccc2c1CN(C1CCC(=O)NC1=O)C2=O]

Step 1: A mixture of 3-(4-bromo-1-oxo-2,3-dihydro-1H-isoindol-2-yl)piperidine-2,6-dione (2.52 mmol), (PPh₃)₂PdCl₂ (0.15 mmol), CuI (0.25 mmol), alkyne (5.04 mmol) were added to a vial. The vial was evacuated and backfilled with N₂ 5 times. DMF and triethylamine (30.3 mmol) were added and the mixture was allowed to stir at 90° C. overnight. The mixture was filtered through celite, washing with MeOH and EtOAc. EtOAc and saturated aqueous NaCl were added. The organic layer was dried with MgSO₄, filtered, concentrated and purified by reverse phase MPLC (5-100% MeCN in H₂O on C18 column) to afford the product.

Step 2: A mixture of disubstituted alkyne (0.81 mmol), Pd/C 10 wt % (0.08 mmol) and EtOH were mixed in a flask. The flask was evacuated and backfilled with H₂ 5 times and allowed to stir at r.t. for 2 h. The mixture was filtered through celite washing with MeOH and EtOAc, concentrated and carried to the next step.

Step 3: A mixture of tert-butylcarbamate (0.81 mmol), CH₂C₁₂ (2 mL), and TFA (2 mL) was allowed to stir at r.t. for 2 h. The mixture was concentrated to afford the amine product.

Step 1 product: tert-butyl N-[2-[2-[2-[3-[2-(2,6-dioxo-3-piperidyl)-1-oxo-isoindolin-4-yl]prop-2-ynoxy]ethoxy]ethoxy]ethyl]carbamate (1.45 g, 58.1%). LCMS: C₂₇H₃₅N₃O₈ requires: 529, found: m/z=552 [M+Na]⁺.

Step 2 product: tert-butyl N-[2-[2-[2-[3-[2-(2,6-dioxo-3-piperidyl)-1-oxo-isoindolin-4-yl]propoxy]ethoxy]ethoxy]ethyl]carbamate (960 mg, 92.75%). LCMS: C₂₇H₃₉N₃O₈ requires: 533, found: m/z=556 [M+Na]⁺.

Step 3 product: 3-[4-[3-[2-[2-(2-aminoethoxy)ethoxy]ethoxy]propyl]-1-oxo-isoindolin-2-yl]piperidine-2,6-dione (576.82 mg, 74.15%). LCMS: C₂₂H₃₁N₃O₆ requires: 433, found: m/z=434 [M+H]⁺.

### Example 31: Synthesis of 5-[(3R)-3-(3-methyl-2-oxoimidazolidin-1-yl)piperidin-1-yl]-3-{[4-(piperazin-1-yl)phenyl]amino}pyrazine-2-carboxamide

### Step 1: 2-(2,6-dioxopiperidin-3-yl)-5-[(2S)-2-(hydroxymethyl)pyrrolidin-1-yl]isoindole-1,3-dione

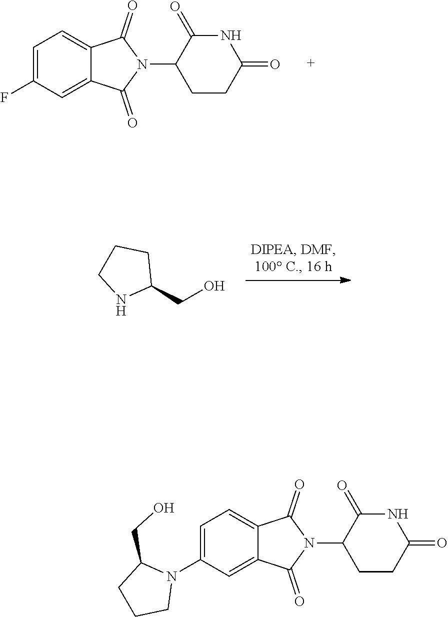 [SMILES: O=C1CCC(N2C(=O)c3ccc(F)cc3C2=O)C(=O)N1 ; OC[C@@H]1CCCN1 ; O=C1CCC(N2C(=O)c3ccc(N4CCC[C@H]4CO)cc3C2=O)C(=O)N1]

A mixture of 2-(2,6-dioxopiperidin-3-yl)-5-fluoroisoindole-1,3-dione (373 mg, 1.35 mmol), DMF (8 mL), ethylbis(propan-2-yl)amine (0.94 mL, 5.40 mmol) and prolinol (137 mg, 1.35 mmol) was allowed to stir at 90° C. for 16 h. CH₂Cl₂ and H₂O were added. The organic layer was dried with MgSO₄, filtered, concentrated and purified by MPLC (0-10% MeOH in CH₂Cl₂) to afford 2-(2,6-dioxopiperidin-3-yl)-5-[(2S)-2-(hydroxymethyl)pyrrolidin-1-yl]isoindole-1,3-dione (386.00 mg, 80.0%).

### Step 2: (2S)-1-[2-(2,6-dioxopiperidin-3-yl)-1-oxo-3H-isoindol-5-yl]pyrrolidine-2-carbaldehyde

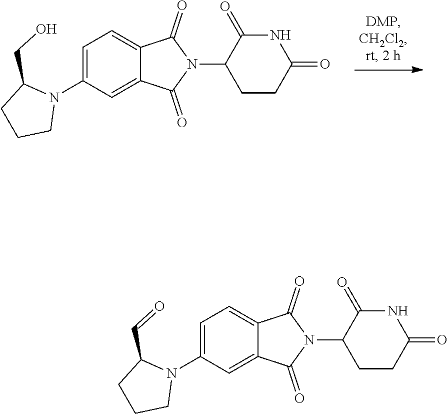 [SMILES: O=C1CCC(N2C(=O)c3ccc(N4CCC[C@H]4CO)cc3C2=O)C(=O)N1 ; O=C[C@@H]1CCCN1c1ccc2c(c1)C(=O)N(C1CCC(=O)NC1=O)C2=O]

1,1-bis(acetyloxy)-3-oxo-1lambda5,2-benziodaoxol-1-yl acetate (548 mg, 1.29 mmol) was added to a mixture of 3-{5-[(2S)-2-(hydroxymethyl)pyrrolidin-1-yl]-1-oxo-3H-isoindol-2-yl}piperidine-2,6-dione (222 mg, 0.65 mmol) and CH₂Cl₂ (10 mL). The mixture was allowed to stir at rt for 1 h. The mixture was purified by MPLC (10-100% EtOAc in hexanes) to afford (2S)-1-[2-(2,6-dioxopiperidin-3-yl)-1-oxo-3H-isoindol-5-yl]pyrrolidine-2-carbaldehyde (67 mg, 30%).

### Example 32: Synthesis of 5-[(3R)-3-(3-methyl-2-oxoimidazolidin-1-yl)piperidin-1-yl]-3-{[6-(piperazin-1-yl)pyridin-3-yl]amino}pyrazine-2-carboxamide

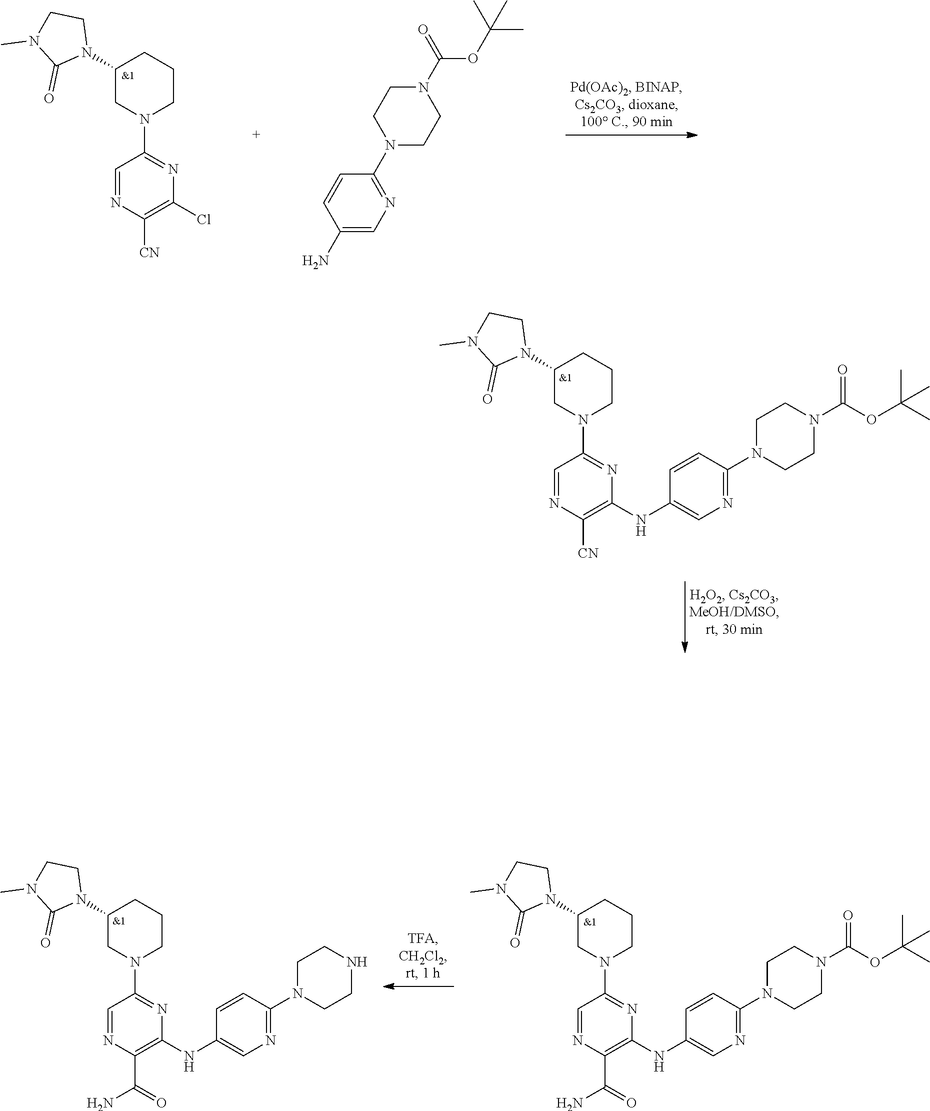 [SMILES: CN1CCN([C@@H]2CCCN(c3cnc(C#N)c(Cl)n3)C2)C1=O ; CC(C)(C)OC(=O)N1CCN(c2ccc(N)cn2)CC1 ; CN1CCN([C@@H]2CCCN(c3cnc(C#N)c(Nc4ccc(N5CCN(C(=O)OC(C)(C)C)CC5)nc4)n3)C2)C1=O ; CN1CCN([C@@H]2CCCN(c3cnc(C(N)=O)c(Nc4ccc(N5CCNCC5)nc4)n3)C2)C1=O ; CN1CCN([C@@H]2CCCN(c3cnc(C(N)=O)c(Nc4ccc(N5CCN(C(=O)OC(C)(C)C)CC5)nc4)n3)C2)C1=O]

### Step 1: tert-butyl 4-[5-({3-cyano-6-[(3R)-3-(3-methyl-2-oxoimidazolidin-1-yl)piperidin-1-yl]pyrazin-2-yl}amino)pyridin-2-yl]piperazine-1-carboxylate

A mixture of 3-chloro-5-[(3R)-3-(3-methyl-2-oxoimidazolidin-1-yl)piperidin-1-yl]pyrazine-2-carbonitrile (322 mg, 1.00 mmol), tert-butyl 4-(5-aminopyridin-2-yl)piperazine-1-carboxylate (293 mg, 1.05 mmol), (acetyloxy)palladio acetate (74 mg, 0.33 mmol), [2′-(diphenylphosphanyl)-[1,1′-binaphthalen]-2-yl]diphenylphosphane (206.27 mg, 0.33 mmol) and Cs₂CO₃ (981 mg, 3.01 mmol) was degassed and backfilled with N₂ 5 times. The mixture was allowed to stir at 100° C. for 90 min. The mixture was filtered through Celite washing with MeOH/EtOAc, concentrated and purified by MPLC (0-100% EtOAc in CH₂Cl₂) to afford tert-butyl 4-[5-({3-cyano-6-[(3R)-3-(3-methyl-2-oxoimidazolidin-1-yl)piperidin-1-yl]pyrazin-2-yl}amino)pyridin-2-yl]piperazine-1-carboxylate (0.2920 g, 51.7%).

### Step 2: tert-butyl 4-[5-({3-carbamoyl-6-[(3R)-3-(3-methyl-2-oxoimidazolidin-1-yl)piperidin-1-yl]pyrazin-2-yl}amino)pyridin-2-yl]piperazine-1-carboxylate

H₂O₂(30% in water, 0.88 mL, 0.09 mmol) was added to a mixture of rac-tert-butyl 4-[5-({3-cyano-6-[(3R)-3-(3-methyl-2-oxoimidazolidin-1-yl)piperidin-1-yl]pyrazin-2-yl}amino)pyridin-2-yl]piperazine-1-carboxylate (292 mg, 0.52 mmol), Cs₂CO₃ (169 mg, 0.52 mmol), DMSO (0.5 mL) and MeOH (10 mL). The mixture was allowed to stir at rt for 30 min. The mixture was concentrated. EtOAc was added and the organic phase was washed with H₂O and brine. The organic layer was dried with MgSO₄, filtered, concentrated and purified by MPLC (0-10% MeOH in CH₂Cl₂) to afford tert-butyl 4-[5-({3-carbamoyl-6-[(3R)-3-(3-methyl-2-oxoimidazolidin-1-yl)piperidin-1-yl]pyrazin-2-yl}amino)pyridin-2-yl]piperazine-1-carboxylate (0.279 g, 92.6%).

### Step 3: 5-[(3R)-3-(3-methyl-2-oxoimidazolidin-1-yl)piperidin-1-yl]-3-{[6-(piperazin-1-yl)pyridin-3-yl]amino}pyrazine-2-carboxamide

A mixture of tert-butyl 4-[5-({3-carbamoyl-6-[(3R)-3-(3-methyl-2-oxoimidazolidin-1-yl)piperidin-1-yl]pyrazin-2-yl}amino)pyridin-2-yl]piperazine-1-carboxylate (279 mg, 0.48 mmol), CH₂C₁₂ (5 mL) and TFA (1 mL) was allowed to stir at rt for 2 h. The volatiles were removed. The mixture was filtered through a NaHCO₃ cartridge, concentrated and purified by reverse phase MPLC (5-90% MeCN in H₂O) to afford 5-[(3R)-3-(3-methyl-2-oxoimidazolidin-1-yl)piperidin-1-yl]-3-{[6-(piperazin-1-yl)pyridin-3-yl]amino}pyrazine-2-carboxamide (0.085 g, 37%).

### Example 33: Synthesis of 1-(2-(2,6-dioxopiperidin-3-yl)-1-oxoisoindolin-5-yl)azetidine-3-carbaldehyde

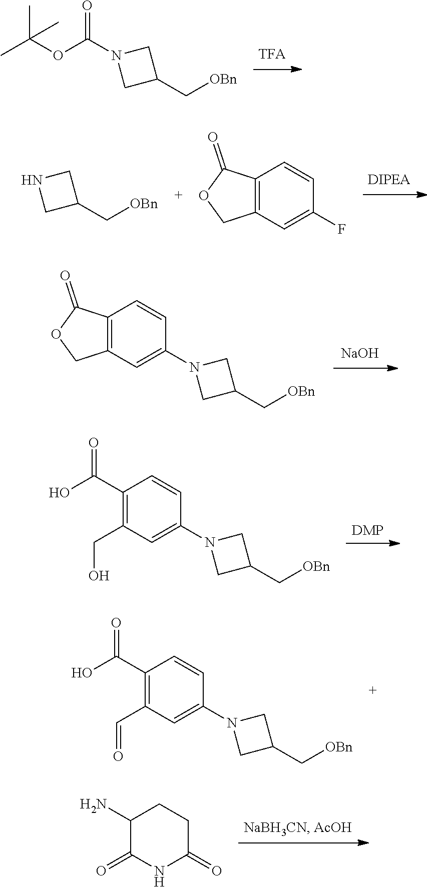

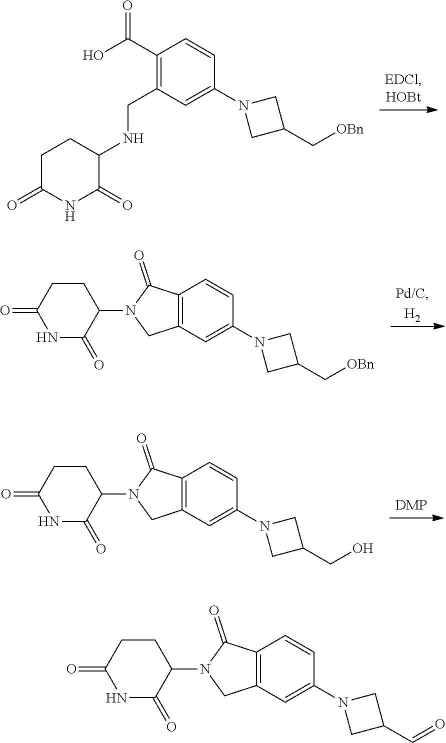

Step 1: TFA (1 mL) was added to a solution of tert-butyl 3-[(benzyloxy)methyl]azetidine-1-carboxylate (1 g, 3.62 mmol, 1.1 eq) in CH₂Cl₂ (1 mL). After stirring for 30 mins, the reaction mixture was concentrated under reduced pressure, and carried to the next step.

Step 2: 5-fluoro-3H-2-benzofuran-1-one (500 mg, 3.29 mmol, 1 eq) and i-Pr₂NEt (2.86 mL, 16.4 mmol, 5 eq) were added sequentially to a solution of crude amine in NMP (4 mL). After stirring at 100° C. for 16 hrs, the reaction was quenched with H₂O. The resulting mixture was extracted with EtOAc, dried over Na₂SO₄, concentrated under reduced pressure. MPLC (0-30% EtOAc in hexanes) afforded the desired product (848 mg, 2.74 mmol, 83% yield). LCMS: C₁₉H₁₉NO₃ requires: 309, found: m/z=310 [M+H]⁺.

Step 3: A solution of NaOH (439 mg, 11 mmol, 4 eq) in H₂O (1.8 mL) was added to a solution of 5-{3-[(benzyloxy)methyl]azetidin-1-yl}-3H-2-benzofuran-1-one (848 mg, 2.74 mmol, 1 eq.) in MeOH (3.4 mL) and THE (3.4 mL). After stirring for 1 hr, the volatile was removed. The resulting mixture was diluted with H₂O, extracted with EtOAc. The aqueous phase was acidified with aqueous 1.5 N HCl to pH 6, extracted with EtOAc, dried over Na₂SO₄, concentrated under reduced pressure, and carried to the next step. (659 mg, 2.01 mmol, 73% yield). LCMS: C₁₉H₂₁NO₄ requires: 327, found: m/z=328 [M+H]⁺.

Step 4: Dess-Martin periodinane (774 mg, 1.83 mmol, 1.1 eq) was added to a solution of 4-{3-[(benzyloxy)methyl]azetidin-1-yl}-2-(hydroxymethyl)benzoic acid (543 mg, 1.66 mmol, 1 eq) in CH₂C₁₂ (8.3 mL). After stirring for 1 hr, the reaction was quenched with an equal mixture of saturated aqueous NaHCO₃ and 10 wt. % aqueous Na₂S₂O₃. After stirring for 30 min, the resulting mixture was extracted with CH₂Cl₂, dried over Na₂SO₄, and concentrated under reduced pressure. MPLC (0-5% MeOH in CH₂Cl₂) afforded the desired product (435 mg, 1.34 mmol, 81% yield). LCMS: C₁₉H₁₉NO₄ requires: 325, found: m/z=326 [M+H]⁺.

Step 5: NaOAc (203 mg, 2.48 mmol, 1.5 eq.) and NaBH₃CN (311 mg, 4.94 mmol, 3 eq.) were added sequentially to a solution of 3-aminopiperidine-2,6-dione hydrochloride (407 mg, 2.48 mmol, 1.5 eq.) and 4-(3-((benzyloxy)methyl)azetidin-1-yl)-2-formylbenzoic acid (536 mg, 1.65 mmol, 1 eq.) in MeOH (8.2 mL). After stirring for 30 mins, the reaction mixture was concentrated under reduced pressure. Reverse phase MPLC (0-70% MeCN in H₂O) afforded the desired product (600 mg, 1.37 mmol, 83% yield). LCMS: C₂₄H₂₇N₃O₅ requires: 437, found: m/z=438 [M+H]⁺.

Step 6: A mixture of 4-(3-((benzyloxy)methyl)azetidin-1-yl)-2-(((2,6-dioxopiperidin-3-yl)amino)methyl)benzoic acid (331 mg, 0.76 mmol, 1 eq), EDCI (176 mg, 1.13 mmol, 1.5 eq), HOBt (174 mg, 1.13 mmol, 1.5 eq) Et₃N (316 μL, 2.27 mmol, 3 eq) in CH₂Cl₂ was allowed to stir at r.t. for 16 hrs. The reaction mixture was washed with H₂O and saturated aqueous NaHCO₃, and concentrated under reduced pressure. MPLC (0-3% MeOH in CH₂C₁₂) afforded the desired product (254 mg, 0.61 mmol, 80% yield). LCMS: C₂₄H₂₅N₃O₄ requires: 419, found: m/z=420 [M+H]⁺.

Step 7: A solution of 3-(5-(3-(hydroxymethyl)azetidin-1-yl)-1-oxoisoindolin-2-yl)piperidine-2,6-dione (100 mg, 0.24 mmol, 1 eq.) in an equal mixture of CH₂C₁₂ (5 mL) and EtOH (5 mL) was stirred with Pd/C (20 mg, 20 wt. %) under a balloon of H₂. After stirring for 16 hrs, the reaction mixture was filtered through Celite, concentrated under reduced pressure, and carried to the next step (79 mg, 0.24 mmol, quantitative). LCMS: C₁₇H₁₉N₃O₄ requires: 329, found: m/z=330 [M+H]⁺.

Step 8: Prepared using general procedure starting from 3-(5-(3-(hydroxymethyl)azetidin-1-yl)-1-oxoisoindolin-2-yl)piperidine-2,6-dione (60 mg, 0.16 mmol) to afford the desired product (23.9 mg, 0.08 mmol, 46%). LCMS: C₁₇H₁₇N₃O₄ requires: 327, found: m/z=328 [M+H]⁺.

### Example 34: Synthesis of (R)-5-(3-(3-methyl-2-oxoimidazolidin-1-yl)piperidin-1-yl)-3-((1-(piperidin-4-yl)-1H-pyrazol-4-yl)amino)pyrazine-2-carboxamide

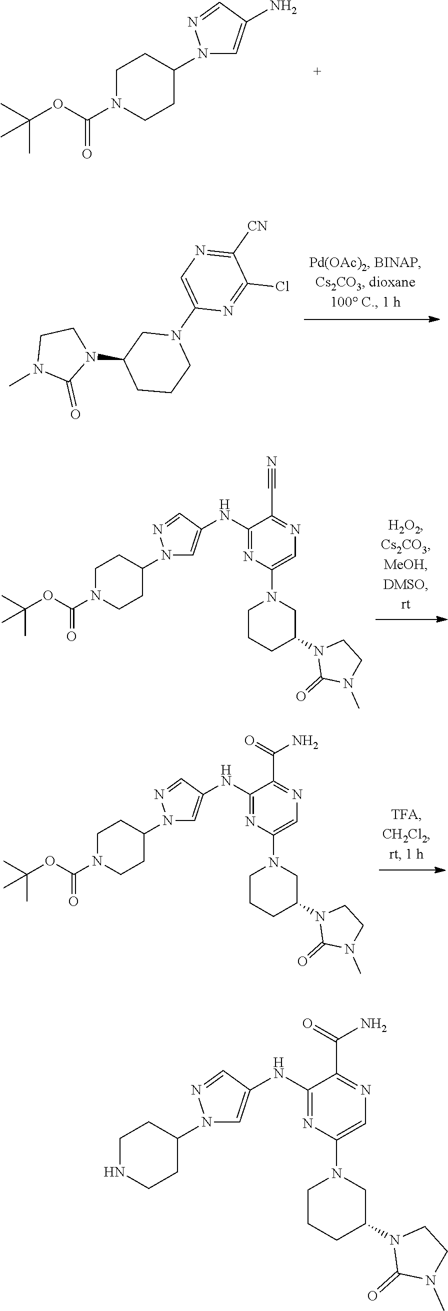 [SMILES: CC(C)(C)OC(=O)N1CCC(n2cc(N)cn2)CC1 ; CN1CCN([C@@H]2CCCN(c3cnc(C#N)c(Cl)n3)C2)C1=O ; CN1CCN([C@@H]2CCCN(c3cnc(C#N)c(Nc4cnn(C5CCN(C(=O)OC(C)(C)C)CC5)c4)n3)C2)C1=O ; CN1CCN([C@@H]2CCCN(c3cnc(C(N)=O)c(Nc4cnn(C5CCN(C(=O)OC(C)(C)C)CC5)c4)n3)C2)C1=O ; CN1CCN([C@@H]2CCCN(c3cnc(C(N)=O)c(Nc4cnn(C5CCNCC5)c4)n3)C2)C1=O]

Prepared in a similar fashion as 5-[(3R)-3-(3-methyl-2-oxoimidazolidin-1-yl)piperidin-1-yl]-3-{[4-(piperazin-1-yl)phenyl]amino}pyrazine-2-carboxamide as described in Example 32.

Step 1: Obtained tert-butyl 4-[4-({3-cyano-6-[(3R)-3-(3-methyl-2-oxoimidazolidin-1-yl)piperidin-1-yl]pyrazin-2-yl}amino)pyrazol-1-yl]piperidine-1-carboxylate (1.45 g, 2.63 mmol, 85%). LCMS: C₂₇H₃₈N₁₀O₃ requires: 550, found: m/z=551 [M+H]⁺.

Step 2: Obtained tert-butyl (R)-4-(4-((3-carbamoyl-6-(3-(3-methyl-2-oxoimidazolidin-1-yl)piperidin-1-yl)pyrazin-2-yl)amino)-1H-pyrazol-1-yl)piperidine-1-carboxylate (1.19 g, 2.09 mmol, 80%). LCMS: C₂₇H₄₀N₁₀O₄ requires: 569, found: m/z=570 [M+H]⁺.

Step 3: Obtained (R)-5-(3-(3-methyl-2-oxoimidazolidin-1-yl)piperidin-1-yl)-3-((1-(piperidin-4-yl)-1H-pyrazol-4-yl)amino)pyrazine-2-carboxamide (106 mg, 0.23 mmol, quantitative). LCMS: C₂₂H₃₂N₁₀O₂ requires: 468, found: m/z=469 [M+H]⁺.

### Example 35: Synthesis of 3-(2-(2,6-dioxopiperidin-3-yl)-3-oxoisoindolin-5-yl)propiolaldehyde

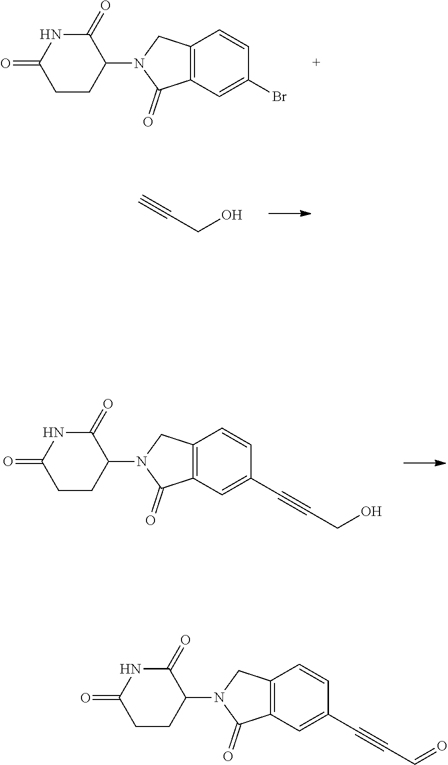

Step 1: Prepared in a similar fashion as tert-butyl 3-({3-[2-(2,6-dioxopiperidin-3-yl)-1,3-dioxoisoindol-5-yl]prop-2-yn-1-yl}oxy)propanoate to afford 3-(6-(3-hydroxyprop-1-yn-1-yl)-1-oxoisoindolin-2-yl)piperidine-2,6-dione (30.7 mg, 0.1 mmol, 11.1%). LCMS: C₁₆H₁₄N₂O₄ requires: 298, found: m/z=299 [M+H]⁺.

Step 2: Prepared in a similar fashion as 1-(2-(2,6-dioxopiperidin-3-yl)-1,3-dioxoisoindolin-5-yl)piperidine-4-carbaldehyde to afford 3-(2-(2,6-dioxopiperidin-3-yl)-3-oxoisoindolin-5-yl)propiolaldehyde. LCMS: C₁₆H₁₂N₂O₄ requires: 296, found: m/z=297 [M+H]⁺.

### Example 36: Synthesis of 3-((4-(1-((1s,3s)-3-aminocyclobutyl)-4-methylpiperidin-4-yl)phenyl)amino)-5-(3-(3-methyl-2-oxoimidazolidin-1-yl)piperidin-1-yl)pyrazine-2-carboxamide and 3-((4-(1-((1r,3r)-3-aminocyclobutyl)-4-methylpiperidin-4-yl)phenyl)amino)-5-(3-(3-methyl-2-oxoimidazolidin-1-yl)piperidin-1-yl)pyrazine-2-carboxamide

### Step 1: tert-butyl ((1s,3s)-3-(4-(4-((3-carbamoyl-6-(3-(3-methyl-2-oxoimidazolidin-1-yl)piperidin-1-yl)pyrazin-2-yl)amino)phenyl)-4-methylpiperidin-1-yl)cyclobutyl)carbamate and tert-butyl ((1r,3r)-3-(4-(4-((3-carbamoyl-6-(3-(3-methyl-2-oxoimidazolidin-1-yl)piperidin-1-yl)pyrazin-2-yl)amino)phenyl)-4-methylpiperidin-1-yl)cyclobutyl)carbamate

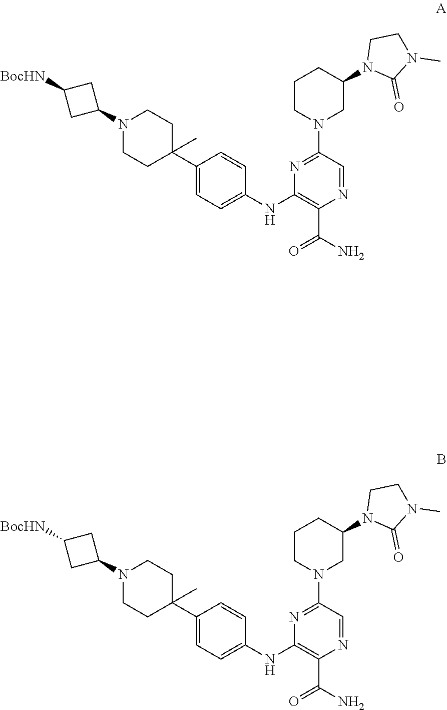 [SMILES: CN1CCN([C@@H]2CCCN(c3cnc(C(N)=O)c(Nc4ccc(C5(C)CCN([C@H]6C[C@@H](NC(=O)OC(C)(C)C)C6)CC5)cc4)n3)C2)C1=O ; CN1CCN([C@@H]2CCCN(c3cnc(C(N)=O)c(Nc4ccc(C5(C)CCN([C@H]6C[C@H](NC(=O)OC(C)(C)C)C6)CC5)cc4)n3)C2)C1=O]

i-Pr₂NEt (115 μL, 0.66 mmol) and tert-butyl N-(3-oxocyclobutyl)carbamate (44 mg, 0.24 mmol) was add to a suspension of 5-[(3R)-3-(3-methyl-2-oxoimidazolidin-1-yl)piperidin-1-yl]-3-{[4-(4-methylpiperidin-4-yl)phenyl]amino}pyrazine-2-carboxamide trifluoroacetic acid (80 mg, 0.13 mmol) in DCE (2 mL). After stirring at RT for 30 min, NaBH(OAc)₃ (37 mg, 0.17 mmol) was added. After stirring at RT overnight, additional NaBH(OAc)₃ (25 mg, 0.12 mmol) was added. After stirring for 3-4 hrs, additional tert-butyl N-(3-oxocyclobutyl)carbamate (10 mg, 0.054 mmol) and NaBH(OAc)₃ was added. After stirring at RT overnight, the yellow reaction mixture was diluted with DCM (50 mL), washed with water, dried over Na₂SO₄, filtered, and concentrated to give a crude yellow liquid. MPLC (0-10% MeOH in DCM) gave separation of two isomers:

Isomer-A (26 mg as a yellow film, 30%). ¹H NMR (500 MHz, CDCl₃) δ 10.92 (s, 1H), 7.64 (d, J=8.4 Hz, 2H), 7.52 (s, 1H), 7.48-7.41 (m, 1H), 7.29 (s, OH), 7.25 (d, J=8.4 Hz, 2H), 5.40-5.16 (m, 2H), 4.38 (dd, J=26.0, 12.8 Hz, 2H), 3.96 (d, J=11.6 Hz, 1H), 3.91-3.79 (m, 1H), 3.51 (s, 1H), 2.84 (s, 3H), 1.44 (s, 10H), 1.27 (s, 4H). LCMS: C₃₅H₅₀N₉O₄ requires: 660, found: m/z=662 [M+H]⁺.

Isomer-B (17 mg of a yellow film, 20%). ¹H NMR (500 MHz, CDCl₃) δ 10.87 (s, 1H), 7.65-7.57 (m, 2H), 7.51 (s, 1H), 7.45 (s, 1H), 7.27 (dd, J=9.7, 2.9 Hz, 2H), 5.29 (s, 1H), 4.80 (s, 1H), 4.37 (d, J=13.3 Hz, 2H), 4.05 (s, 1H), 3.93-3.78 (m, 1H), 3.50 (s, 3H), 3.41 (dd, J=7.5, 5.8 Hz, 1H), 3.38-3.27 (m, 3H), 3.11 (dd, J=12.8, 10.5 Hz, 1H), 3.03-2.93 (m, 2H), 2.84 (s, 3H), 2.44 (t, J=52.0 Hz, 5H), 2.20 (s, 2H), 2.12-1.98 (m, OH), 1.97-1.63 (m, 2H), 1.24 (s, 3H). LCMS: C₃₅H₅₀N₉O₄ requires: 660, found: m/z=662 [M+H]⁺.

### Step 2: 3-((4-(1-((1s,3s)-3-aminocyclobutyl)-4-methylpiperidin-4-yl)phenyl)amino)-5-(3-(3-methyl-2-oxoimidazolidin-1-yl)piperidin-1-yl)pyrazine-2-carboxamide and 3-((4-(1-((1r,3r)-3-aminocyclobutyl)-4-methylpiperidin-4-yl)phenyl)amino)-5-(3-(3-methyl-2-oxoimidazolidin-1-yl)piperidin-1-yl)pyrazine-2-carboxamide

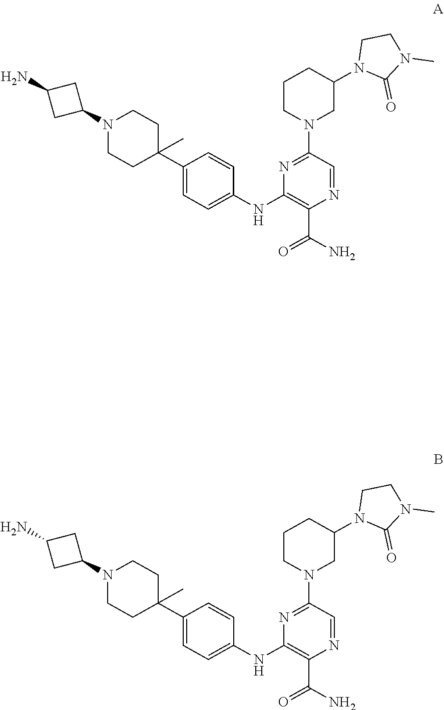 [SMILES: CN1CCN(C2CCCN(c3cnc(C(N)=O)c(Nc4ccc(C5(C)CCN([C@H]6C[C@@H](N)C6)CC5)cc4)n3)C2)C1=O ; CN1CCN(C2CCCN(c3cnc(C(N)=O)c(Nc4ccc(C5(C)CCN([C@H]6C[C@H](N)C6)CC5)cc4)n3)C2)C1=O]

To a solution of tert-butyl N-[(1R,3R)-3-{4-[4-({3-carbamoyl-6-[(3R)-3-(3-methyl-2-oxoimidazolidin-1-yl)piperidin-1-yl]pyrazin-2-yl}amino)phenyl]-4-methylpiperidin-1-yl}cyclobutyl]carbamate (17 mg, 0.03 mmol) in DCM (1 mL) was added trifluoroacetic acid (65.38 μL, 0.10 g, 0.85 mmol). After stirring for 30 min at RT, the reaction mixture was concentrated, re-dissolved in DCM and re-concentrated.

### Example 37: Synthesis of tert-butyl 7-(4-nitrophenyl)-2,7-diazaspiro[3.5]nonane-2-carboxylate

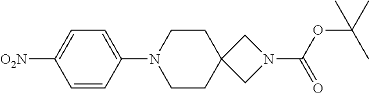 [SMILES: CC(C)(C)OC(=O)N1CC2(CCN(c3ccc([N+](=O)[O-])cc3)CC2)C1]

Tert-butyl 2,7-diazaspiro[3.5]nonane-2-carboxylate (419 mg, 1.85 mmol), 4-fluoronitrobenzene (261 mg, 1.85 mmol) and potassium carbonate (511 mg, 3.70 mmol) were stirred in DMF (5.00 mL) at 90° C. overnight. 30 mL water was added. The resulting solid was filtered and washed with water then air dried overnight to provide tert-butyl 7-(4-nitrophenyl)-2,7-diazaspiro[3.5]nonane-2-carboxylate (606 mg, 94.2%). LCMS: C₁₈H₂₅N₃O₄ requires 347, found: m/z=348 [M+H]⁺.

### Example 38: Synthesis of tert-butyl 7-(4-aminophenyl)-2,7-diazaspiro[3.5]nonane-2-carboxylate

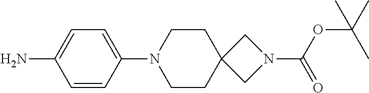 [SMILES: CC(C)(C)OC(=O)N1CC2(CCN(c3ccc(N)cc3)CC2)C1]

Tert-butyl 7-(4-nitrophenyl)-2,7-diazaspiro[3.5]nonane-2-carboxylate (606 mg, 1.74 mmol) and 10% Pd/C (50 mg, mmol) were stirred in EtOH (3.00 mL) and ethyl acetate (3.00 mL) under a balloon of H₂. After 2 hours, 10% Pd/C (50 mg, mmol) was added. The mixture stirred under a balloon of H₂ overnight then was filtered through a plug of celite and concentrated to provide tert-butyl 7-(4-aminophenyl)-2,7-diazaspiro[3.5]nonane-2-carboxylate (545 mg, 98.4%). LCMS: C₁₈H₂₇N₃O₂ requires 317, found: m/z=318 [M+H]⁺.

### Example 39: Synthesis of tert-butyl 7-[4-({3-cyano-6-[(3R)-3-(3-methyl-2-oxoimidazolidin-1-yl)piperidin-1-yl]pyrazin-2-yl}amino)phenyl]-2,7-diazaspiro[3.5]nonane-2-carboxylate

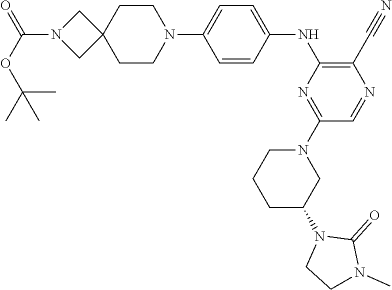 [SMILES: CN1CCN([C@@H]2CCCN(c3cnc(C#N)c(Nc4ccc(N5CCC6(CC5)CN(C(=O)OC(C)(C)C)C6)cc4)n3)C2)C1=O]

3-Chloro-5-[(3R)-3-(3-methyl-2-oxoimidazolidin-1-yl)piperidin-1-yl]pyrazine-2-carbonitrile (209 mg, 0.65 mmol), tert-butyl 7-(4-aminophenyl)-2,7-diazaspiro[3.5]nonane-2-carboxylate (207 mg, 0.65 mmol), and cesium carbonate (0.85 g, 2.61 mmol) were deposited in a vial with dioxane (6.00 mL). A vacuum was pulled on the vial until the mixture bubbled and the headspace was backfilled with argon 5 times. Palladium (II) acetate (29 mg, 0.13 mmol) and BINAP (81 mg, 0.13 mmol) were added. A vacuum was pulled on the vial and the headspace was backfilled with argon for 5 cycles. The mixture was heated at 90° C. overnight. Water was added and the mixture was extracted twice with DCM. The combined organic layers were concentrated then purified by flash chromatography on a 24 g column eluted with 0 to 10% MeOH/ethyl acetate to provide tert-butyl 7-[4-({3-cyano-6-[(3R)-3-(3-methyl-2-oxoimidazolidin-1-yl)piperidin-1-yl]pyrazin-2-yl}amino)phenyl]-2,7-diazaspiro[3.5]nonane-2-carboxylate (258 mg, 65.8%). LCMS: C₃₂H₄₃N₉O₃ requires 601, found: m/z=602 [M+H]⁺.

### Example 40: Synthesis of tert-butyl 7-[4-({3-carbamoyl-6-[(3R)-3-(3-methyl-2-oxoimidazolidin-1-yl)piperidin-1-yl]pyrazin-2-yl}amino)phenyl]-2,7-diazaspiro[3.5]nonane-2-carboxylate

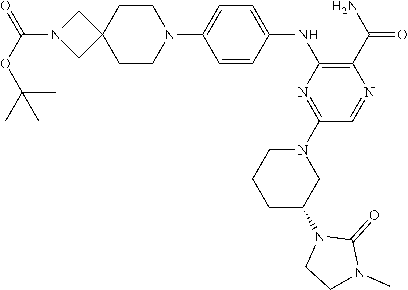 [SMILES: CN1CCN([C@@H]2CCCN(c3cnc(C(N)=O)c(Nc4ccc(N5CCC6(CC5)CN(C(=O)OC(C)(C)C)C6)cc4)n3)C2)C1=O]

Tert-butyl 7-[4-({3-cyano-6-[(3R)-3-(3-methyl-2-oxoimidazolidin-1-yl)piperidin-1-yl]pyrazin-2-yl}amino)phenyl]-2,7-diazaspiro[3.5]nonane-2-carboxylate (258 mg, 0.43 mmol) was dissolved in MeOH (6.00 mL) and DMSO (3.00 mL). Cesium carbonate (140 mg, 0.43 mmol) and 1 mL 35% H₂O₂ were added. After 1 hour, 3 mL ACN was added. After 5 minutes, the mixture got hot. Water and ethyl acetate were added. The organic layer was washed with 2 more portions of water. The organic layer was dried over Na₂SO₄ and concentrated to provide tert-butyl 7-[4-({3-carbamoyl-6-[(3R)-3-(3-methyl-2-oxoimidazolidin-1-yl)piperidin-1-yl]pyrazin-2-yl}amino)phenyl]-2,7-diazaspiro[3.5]nonane-2-carboxylate (267 mg, 100%). LCMS: C₃₂H₄₅N₉O₄ requires 619, found: m/z=620 [M+H]⁺.

### Example 41: Synthesis of 3-[(4-{2,7-diazaspiro[3.5]nonan-7-yl}phenyl)amino]-5-[(3R)-3-(3-methyl-2-oxoimidazolidin-1-yl)piperidin-1-yl]pyrazine-2-carboxamide

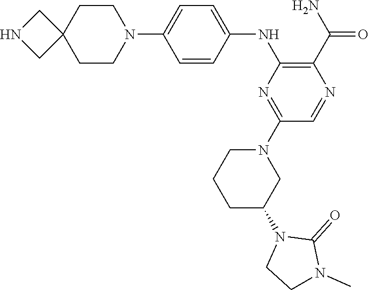 [SMILES: CN1CCN([C@@H]2CCCN(c3cnc(C(N)=O)c(Nc4ccc(N5CCC6(CC5)CNC6)cc4)n3)C2)C1=O]

Tert-butyl 7-[4-({3-carbamoyl-6-[(3R)-3-(3-methyl-2-oxoimidazolidin-1-yl)piperidin-1-yl]pyrazin-2-yl}amino)phenyl]-2,7-diazaspiro[3.5]nonane-2-carboxylate (267 mg, 0.43 mmol) was stirred in DCM (2.00 mL) and TFA (2.00 mL) for 15 minutes. The mixture was concentrated to provide 3-[(4-{2,7-diazaspiro[3.5]nonan-7-yl}phenyl)amino]-5-[(3R)-3-(3-methyl-2-oxoimidazolidin-1-yl)piperidin-1-yl]pyrazine-2-carboxamide (223 mg, 100%). LCMS: C₂₇H₃₇N₉O₂ requires 519, found: m/z=520 [M+H]⁺.

### Example 42: Synthesis of 3-[(4-{2,6-diazaspiro[3.5]nonan-6-yl}phenyl)amino]-5-[(3R)-3-(3-methyl-2-oxoimidazolidin-1-yl)piperidin-1-yl]pyrazine-2-carboxamide

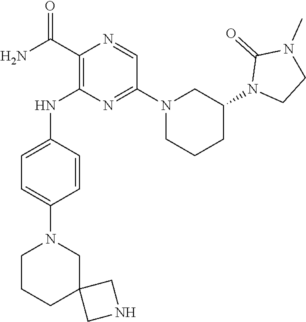 [SMILES: CN1CCN([C@@H]2CCCN(c3cnc(C(N)=O)c(Nc4ccc(N5CCCC6(CNC6)C5)cc4)n3)C2)C1=O]

3-[(4-{2,6-diazaspiro[3.5]nonan-6-yl}phenyl)amino]-5-[(3R)-3-(3-methyl-2-oxoimidazolidin-1-yl)piperidin-1-yl]pyrazine-2-carboxamide was made in an analogous fashion to 3-[(4-{2,7-diazaspiro[3.5]nonan-7-yl}phenyl)amino]-5-[(3R)-3-(3-methyl-2-oxoimidazolidin-1-yl)piperidin-1-yl]pyrazine-2-carboxamide starting with tert-butyl 2,6-diazaspiro[3.5]nonane-2-carboxylate (428 mg, 1.89 mmol). LCMS: C₂₇H₃₇N₉O₂ requires 519, found: m/z=520 [M+H]⁺.

### Example 43: Synthesis of tert-butyl 4-[6-({3-cyano-6-[(3R)-3-(3-methyl-2-oxoimidazolidin-1-yl)piperidin-1-yl]pyrazin-2-yl]amino)pyridin-3-yl}piperazine-1-carboxylate

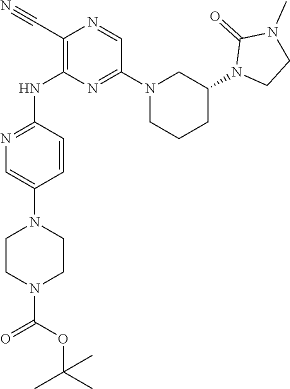 [SMILES: CN1CCN([C@@H]2CCCN(c3cnc(C#N)c(Nc4ccc(N5CCN(C(=O)OC(C)(C)C)CC5)cn4)n3)C2)C1=O]

3-Chloro-5-[(3R)-3-(3-methyl-2-oxoimidazolidin-1-yl)piperidin-1-yl]pyrazine-2-carbonitrile (218 mg, 0.68 mmol), tert-butyl 4-(6-aminopyridin-3-yl)piperazine-1-carboxylate (284 mg, 1.02 mmol), and cesium carbonate (886 mg, 2.72 mmol) were deposited in a vial with dioxane (5.00 mL). A vacuum was pulled on the vial and the headspace was backfilled with argon for 5 cycles. Palladium (II) acetate (31 mg, 0.14 mmol) and BINAP (85 mg, 0.14 mmol) were added. A vacuum was pulled and the headspace was backfilled with argon for 5 cycles. The mixture was next heated at 90° C. overnight. The mixture was cooled, diluted with DCM, filtered, and concentrated. The crude residue was purified by flash chromatography on a 24 g column eluted with 0 to 10% MeOH/DCM to provide tert-butyl 4-[6-({3-cyano-6-[(3R)-3-(3-methyl-2-oxoimidazolidin-1-yl)piperidin-1-yl]pyrazin-2-yl}amino)pyridin-3-yl]piperazine-1-carboxylate (282 mg, 73.7%). LCMS: C₂₈H₃₈N₁₀O₃ requires 562, found: m/z=563 [M+H]⁺.

### Example 44: Synthesis of tert-butyl 4-[6-({3-carbamoyl-6-[(3R)-3-(3-methyl-2-oxoimidazolidin-1-yl)piperidin-1-yl]pyrazin-2-yl]amino)pyridin-3-yl}piperazine-1-carboxylate

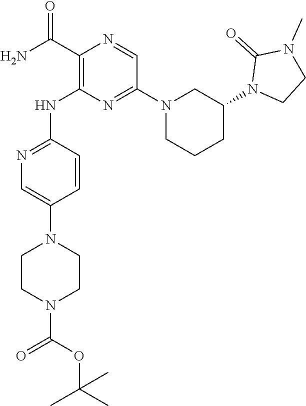 [SMILES: CN1CCN([C@@H]2CCCN(c3cnc(C(N)=O)c(Nc4ccc(N5CCN(C(=O)OC(C)(C)C)CC5)cn4)n3)C2)C1=O]

To a mixture of tert-butyl 4-[6-({3-cyano-6-[(3R)-3-(3-methyl-2-oxoimidazolidin-1-yl)piperidin-1-yl]pyrazin-2-yl}amino)pyridin-3-yl]piperazine-1-carboxylate (280 mg, 0.50 mmol) and cesium carbonate (162 mg, 0.50 mmol) in MeOH (6.00 mL) and DMSO (3.00 mL) was added 1 mL 35% hydrogen peroxide. After 5 hours, 3 mL acetonitrile was added. After 5 minutes, the mixture became hot. The mixture was transferred to a separatory funnel with ethyl acetate and was washed with water 3×. The organic layer was dried over Na₂SO₄ and concentrated. The crude residue was purified on a 24 g flash column eluted with 0 to 20% MeOH/ethyl acetate to provide tert-butyl 4-[6-({3-carbamoyl-6-[(3R)-3-(3-methyl-2-oxoimidazolidin-1-yl)piperidin-1-yl]pyrazin-2-yl}amino)pyridin-3-yl]piperazine-1-carboxylate (191 mg, 66.1%). LCMS: C₂₈H₄₀N₁₀O₄ requires 580, found: m/z=581 [M+H]⁺.

### Example 45: 5-[(3R)-3-(3-methyl-2-oxoimidazolidin-1-yl)piperidin-1-yl]-3-{[5-(piperazin-1-yl)pyridin-2-yl]amino}pyrazine-2-carboxamide

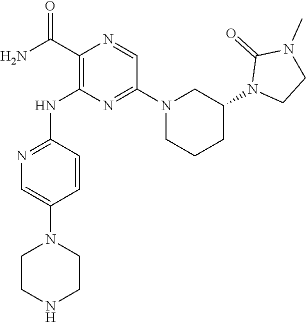 [SMILES: CN1CCN([C@@H]2CCCN(c3cnc(C(N)=O)c(Nc4ccc(N5CCNCC5)cn4)n3)C2)C1=O]

To tert-butyl 4-[6-({3-carbamoyl-6-[(3R)-3-(3-methyl-2-oxoimidazolidin-1-yl)piperidin-1-yl]pyrazin-2-yl}amino)pyridin-3-yl]piperazine-1-carboxylate (40 mg, 0.07 mmol) was added 4M hydrogen chloride solution in dioxane(1.00 mL, 0.15 g, 4.00 mmol) and DCM (1.00 mL). After 20 minutes, the mixture was concentrated to provide 5-[(3R)-3-(3-methyl-2-oxoimidazolidin-1-yl)piperidin-1-yl]-3-{[5-(piperazin-1-yl)pyridin-2-yl]amino}pyrazine-2-carboxamide (33 mg, 100%). LCMS: C₂₃H₃₂N₁₀O₂ requires 480, found: m/z=481 [M+H]⁺.

### Example 46: Synthesis of tert-butyl 2-({3-cyano-6-[(3R)-3-(3-methyl-2-oxoimidazolidin-1-yl)piperidin-1-yl]pyrazin-2-yl}amino)-4H,6H,7H-pyrazolo[1,5-a]pyrazine-5-carboxylate

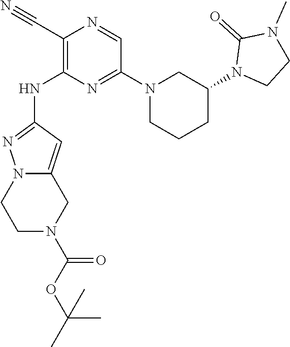 [SMILES: CN1CCN([C@@H]2CCCN(c3cnc(C#N)c(Nc4cc5n(n4)CCN(C(=O)OC(C)(C)C)C5)n3)C2)C1=O]

3-chloro-5-[(3R)-3-(3-methyl-2-oxoimidazolidin-1-yl)piperidin-1-yl]pyrazine-2-carbonitrile (299 mg, 0.93 mmol), tert-butyl 2-amino-4H,6H,7H-pyrazolo[1,5-a]pyrazine-5-carboxylate (222 mg, 0.93 mmol), and cesium carbonate (1.21 g, 3.73 mmol) were deposited in a vial with dioxane (6.00 mL). A vacuum was pulled on the vial until the mixture bubbled then the headspace was backfilled with argon for 5 cycles. BINAP (116 mg, 0.19 mmol) and palladium (II) acetate (42 mg, 0.19 mmol) were added. A vacuum was pulled on the vial until the mixture bubbled then the headspace was backfilled with argon for 5 cycles. The mixture was heated at 90° C. overnight. The mixture was diluted with DCM and filtered. The resulting solution was concentrated and purified by flash chromatography on a 40 g column eluted with 0 to 10% MeOH/ethyl acetate to provide tert-butyl 2-({3-cyano-6-[(3R)-3-(3-methyl-2-oxoimidazolidin-1-yl)piperidin-1-yl]pyrazin-2-yl}amino)-4H,6H,7H-pyrazolo[1,5-a]pyrazine-5-carboxylate (0.285 g, 58.5%). LCMS: C₂₅H₃₄N₁₀O₃ requires 522 found: m/z=523 [M+H]⁺.

### Example 47: Synthesis of tert-butyl 2-({3-carbamoyl-6-[(3R)-3-(3-methyl-2-oxoimidazolidin-1-yl)piperidin-1-yl]pyrazin-2-yl}amino)-4H,6H,7H-pyrazolo[1,5-a]pyrazine-5-carboxylate

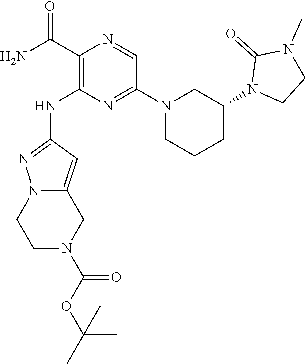 [SMILES: CN1CCN([C@@H]2CCCN(c3cnc(C(N)=O)c(Nc4cc5n(n4)CCN(C(=O)OC(C)(C)C)C5)n3)C2)C1=O]

To a mixture of tert-butyl 2-({3-cyano-6-[(3R)-3-(3-methyl-2-oxoimidazolidin-1-yl)piperidin-1-yl]pyrazin-2-yl}amino)-4H,6H,7H-pyrazolo[1,5-a]pyrazine-5-carboxylate (285 mg, 0.55 mmol) in MeOH (6.00 mL) and DMSO (3.00 mL) was added cesium carbonate (178 mg, 0.55 mmol) followed by 35% hydrogen peroxide (0.10 mL, 0.04 g, 1.09 mmol). After 40 minutes, the reaction was quenched with 3 mL acetonitrile. The mixture was transferred to a separatory funnel with ethyl acetate and was washed twice with water. The organic layer was dried over Na₂SO₄ and concentrated to provide tert-butyl 2-({3-carbamoyl-6-[(3R)-3-(3-methyl-2-oxoimidazolidin-1-yl)piperidin-1-yl]pyrazin-2-yl}amino)-4H,6H,7H-pyrazolo[1,5-a]pyrazine-5-carboxylate (0.295 g, 100%). LCMS: C₂₅H₃₆N₁₀O₄ requires 540, found: m/z=541 [M+H]⁺.

### Example 48: Synthesis of 5-[(3R)-3-(3-methyl-2-oxoimidazolidin-1-yl)piperidin-1-yl]-3-{4H,5H,6H,7H-pyrazolo[1,5-a]pyrazin-2-ylamino}pyrazine-2-carboxamide

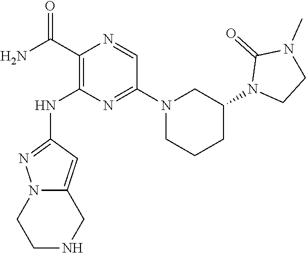 [SMILES: CN1CCN([C@@H]2CCCN(c3cnc(C(N)=O)c(Nc4cc5n(n4)CCNC5)n3)C2)C1=O]

tert-butyl 2-({3-carbamoyl-6-[(3R)-3-(3-methyl-2-oxoimidazolidin-1-yl)piperidin-1-yl]pyrazin-2-yl}amino)-4H,6H,7H-pyrazolo[1,5-a]pyrazine-5-carboxylate (24 mg, 0.04 mmol) was stirred in DCM (1.00 mL) and TFA (1.00 mL) for 20 minutes. The mixture was concentrated to provide 5-[(3R)-3-(3-methyl-2-oxoimidazolidin-1-yl)piperidin-1-yl]-3-{4H,5H,6H,7H-pyrazolo[1,5-a]pyrazin-2-ylamino}pyrazine-2-carboxamide (0.020 g, 100%). LCMS: C₂₀H₂₈N₁₀O₂ requires 440, found: m/z=441 [M+H]⁺.

### Example 49: Synthesis of 5-((R)-3-(3-methyl-2-oxoimidazolidin-1-yl)piperidin-1-yl)-3-((4-(octahydro-2,7-naphthyridin-2(1H)-yl)phenyl)amino)pyrazine-2-carboxamide

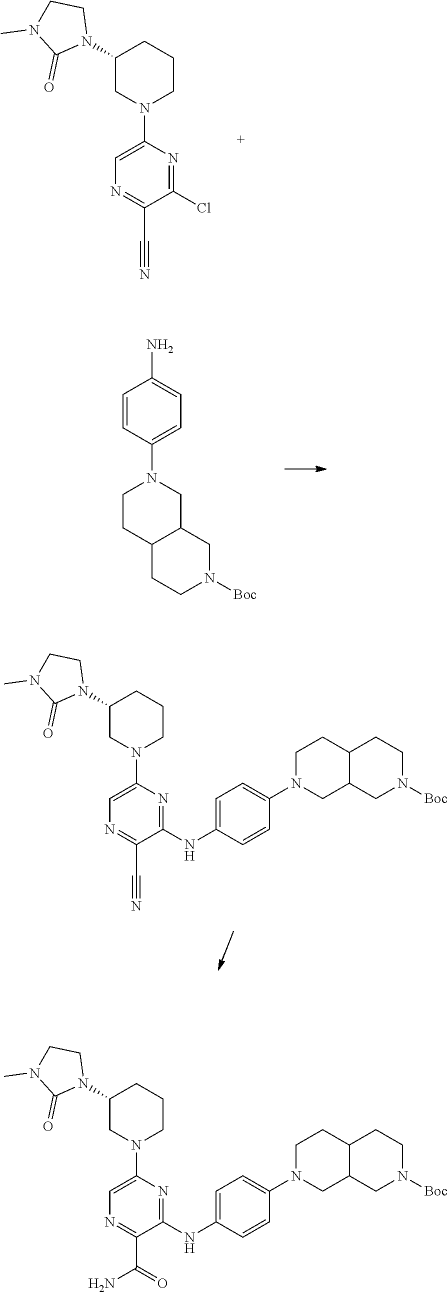 [SMILES: CN1CCN([C@@H]2CCCN(c3cnc(C#N)c(Cl)n3)C2)C1=O ; CC(C)(C)OC(=O)N1CCC2CCN(c3ccc(N)cc3)CC2C1 ; CN1CCN([C@@H]2CCCN(c3cnc(C#N)c(Nc4ccc(N5CCC6CCN(C(=O)OC(C)(C)C)CC6C5)cc4)n3)C2)C1=O ; CN1CCN([C@@H]2CCCN(c3cnc(C(N)=O)c(Nc4ccc(N5CCC6CCN(C(=O)OC(C)(C)C)CC6C5)cc4)n3)C2)C1=O]

Step 1: Under argon, Pd(OAc)₂ (105 mg, 0.47 mmol) was added to a degassed dioxane (10.00 mL) solution containing cesium carbonate (1523.56 mg, 4.68 mmol), tert-butyl 7-(4-aminophenyl)-octahydro-2,7-naphthyridine-2-carboxylate (517 mg, 1.56 mmol), BINAP (291 mg, 0.47 mmol), and 3-chloro-5-[(3R)-3-(3-methyl-2-oxoimidazolidin-1-yl)piperidin-1-yl]pyrazine-2-carbonitrile (500 mg, 1.56 mmol). The mixture was then stirred at 100 deg. C. for 16 h. The mixture was then partition with water and ethyl acetate, dried over sodium sulfate, and concentrated. The resulting residue was then purified by reverse phase preparative HPLC (Waters 5 mM CSH C18 column, 50×50 mm), eluting with solvent with acetonitrile in water with 0.1% TFA, using a 10-95% gradient over 9 min. The desired fractions were combined and concentrated to give product. This material was dissolved in a MeOH/DMSO solution (2 mL) 10:1 with one NaOH pellet. After 2 min. a 30% aqueous hydrogen peroxide solution (0.5 mL) was added and the reaction continued stirring at room temp for 1 h. The reaction was quenched with the addition of ACN. After concentration the crude reaction mixture was then purified by reverse phase preparative HPLC (Waters 5 mM CSH C18 column, 50×50 mm), eluting with solvent with acetonitrile in water with 0.1% TFA, using a 10-95% gradient over 9 min. The desired fractions were combined and concentrated to give product. LCMS C₂₅H₃₄N₆O₃ requires 633, found: m/z=634 [M+H]⁺. PGP-478 C₃

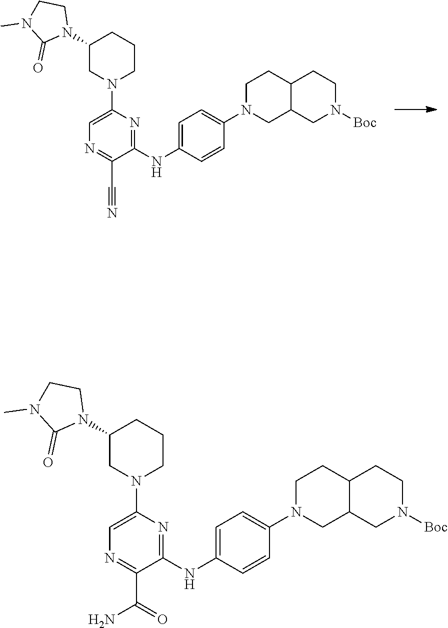 [SMILES: CN1CCN([C@@H]2CCCN(c3cnc(C#N)c(Nc4ccc(N5CCC6CCN(C(=O)OC(C)(C)C)CC6C5)cc4)n3)C2)C1=O ; CN1CCN([C@@H]2CCCN(c3cnc(C(N)=O)c(Nc4ccc(N5CCC6CCN(C(=O)OC(C)(C)C)CC6C5)cc4)n3)C2)C1=O]

Step 2: Tert-butyl 7-[4-({3-carbamoyl-6-[(3R)-3-(3-methyl-2-oxoimidazolidin-1-yl)piperidin-1-yl]pyrazin-2-yl}amino)phenyl]-octahydro-2,7-naphthyridine-2-carboxylate (240 mg, 0.38 mmol) was dissolved in 1/1 DCM/TFA solution 2 mL and stirred at room temp for 1 h. The reaction was then concentrated. This material was used in the next step without further purification.

### Example 50A: Synthesis of (R)-3-((6-(3,9-diazaspiro[5.5]undecan-3-yl)pyridin-3-yl)amino)-5-(3-(3-methyl-2-oxoimidazolidin-1-yl)piperidin-1-yl)pyrazine-2-carboxamide

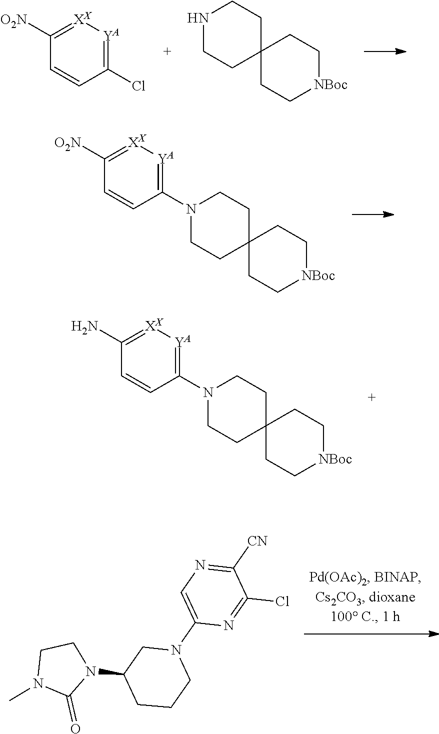

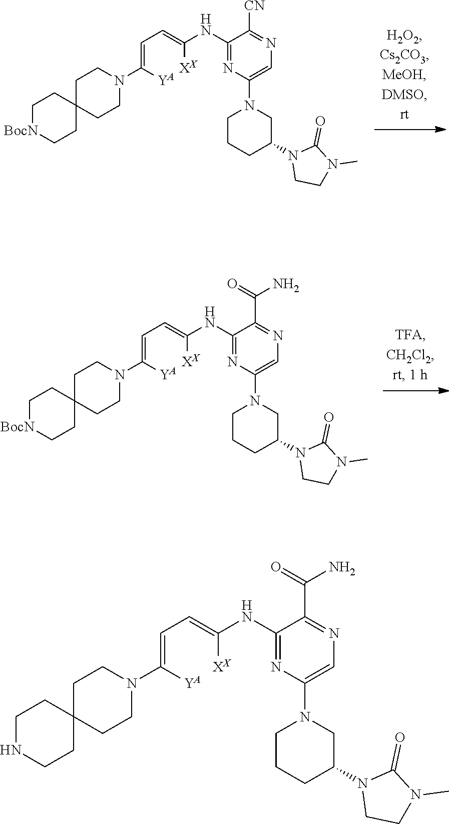

wherein X^{X} and Y^{A} are each independently CH or N.

Step 1: 2-Chloro-5-nitropyridine (1 eq; Y^{A} is N and X^{X} is CH) and tert-butyl 3,9-diazaspiro[5.5]undecane-3-carboxylate (1 eq) were combined in DMF:DIEA solution (10:1 ratio, 0.1M). The reaction mixture was stirred at 70° C. for 16 h, then cooled to room temperature. The reaction mixture was then partitioned between ethyl acetate and water, and the organic layer was separated, dried over magnesium sulfate, and filtered. This solution was concentrated onto silica gel and chromatographed by silica (0-100% ethyl acetate in hexane) to afford tert-butyl 9-(5-nitropyridin-2-yl)-3,9-diazaspiro[5.5]undecane-3-carboxylate (90%). LCMS C₁₉H₂₈N₄O₄ requires: 376.5 found: m/z=377.4 [M+H]⁺.

Step 2: The purified material from step 1 was dissolved in ethanol and water (10:1). Ammonium chloride (3.5 eq) and iron (3 eq) were added, followed by vigorous stirring and heating to 90° C. for 9 h. The reaction was then filtered with Celite while still hot, and the Celite was further washed with ethyl acetate. The resulting solution was partitioned between ethyl acetate and water. The water layer was separated and re-extracted with ethyl acetate. The combined organic layers were washed with brine, dried over magnesium sulfate, and concentrated. Silica gel chromatography provided tert-butyl 9-(5-aminopyridin-2-yl)-3,9-diazaspiro [5.5]undecane-3-carboxylate (85%). LCMS C₁₉H₃₀N₄O₂ requires: 346.5, found: m/z=347.4 [M+H]⁺.

Step 3: Pd(OAc)₂ (73 mg) was added to a degassed dioxane (10.00 mL) solution containing cesium carbonate (1.78 g), tert-butyl 9-(5-aminopyridin-2-yl)-3,9-diazaspiro [5.5]undecane-3-carboxylate (1.134 g), BINAP (200 mg), and 3-chloro-5-[(3R)-3-(3-methyl-2-oxoimidazolidin-1-yl)piperidin-1-yl]pyrazine-2-carbonitrile (700 mg). The mixture was then stirred at 100° C. for 4 h. The mixture was filtered and purified by MPLC (0-10% MeOH in CH₂Cl₂) to afford tert-butyl (R)-9-(5-((3-cyano-6-(3-(3-methyl-2-oxoimidazolidin-1-yl)piperidin-1-yl)pyrazin-2-yl)amino)pyridin-2-yl)-3,9-diazaspiro[5.5]undecane-3-carboxylate (800 mg). LCMS C₃₃H₄₆N₁₀O₃ requires: 630, found: m/z=631 [M+H]⁺.

Step 4: Starting material was dissolved in methanol/DMSO, followed by addition of 3 pellets of NaOH (solid). The reaction was stirred for 1 minute before addition of 5 mL of 30% aq hydrogen peroxide solution. The reaction was stirred for 1 h. The reaction mixture was then partitioned between ethyl acetate and water. The organic layer was separated, and washed with water, then brine. The mixture was purified by MPLC (0-10% MeOH in CH₂C₁₂) to afford tert-butyl (R)-9-(5-((3-carbamoyl-6-(3-(3-methyl-2-oxoimidazolidin-1-yl)piperidin-1-yl)pyrazin-2-yl)amino)pyridin-2-yl)-3,9-diazaspiro[5.5]undecane-3-carboxylate (750 mg, 91%). LCMS C₃₃H₄₈N₁₀O₄ requires: 648, found: m/z=649 [M+H]⁺.

Step 5: A mixture of TFA (2 mL), CH₂Cl₂(15 mL) and tert-butyl (R)-9-(5-((3-carbamoyl-6-(3-(3-methyl-2-oxoimidazolidin-1-yl)piperidin-1-yl)pyrazin-2-yl)amino)pyridin-2-yl)-3,9-diazaspiro[5.5]undecane-3-carboxylate (750 mg) was allowed to stir at rt for 1 h. The volatiles were removed and the material was carried to the next step. LCMS C₂₈H₄₀N₁₀O₂ requires: 548, found: m/z=549 [M+H]⁺.

### Example 50B: Synthesis of (R)-3-((5-(3,9-diazaspiro[5.5]undecan-3-yl)pyridin-2-yl)amino)-5-(3-(3-methyl-2-oxoimidazolidin-1-yl)piperidin-1-yl)pyrazine-2-carboxamide

The title compound was synthesized using the procedure in Example 50A, but substituting 5-Chloro-2-nitropyridine, wherein Y^{A} is CH and X^{X} is N, for 2-chloro-5-nitropyridine. LCMS C₂₈H₄₀N₁₀O₂ requires: 548, found: m/z=549 [M+H]⁺.

### Example 51: Synthesis of 3-((4-(hexahydropyrrolo[3,4-c]pyrrol-2(1H)-yl)phenyl)amino)-5-((R)-3-(3-methyl-2-oxoimidazolidin-1-yl)piperidin-1-yl)pyrazine-2-carboxamide

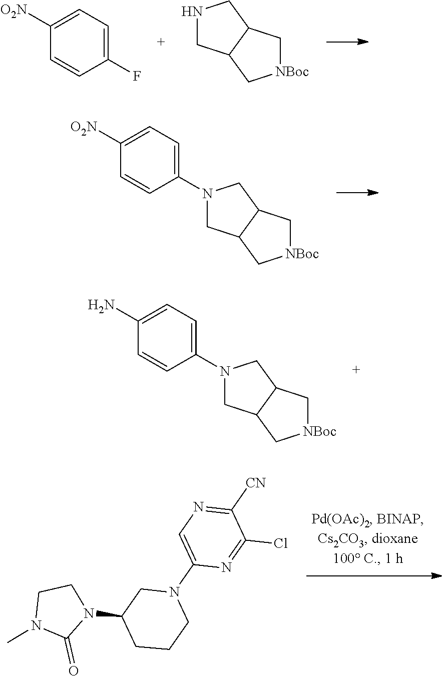 [SMILES: O=[N+]([O-])c1ccc(F)cc1 ; CC(C)(C)OC(=O)N1CC2CNCC2C1 ; CC(C)(C)OC(=O)N1CC2CN(c3ccc([N+](=O)[O-])cc3)CC2C1 ; CC(C)(C)OC(=O)N1CC2CN(c3ccc(N)cc3)CC2C1 ; CN1CCN([C@@H]2CCCN(c3cnc(C#N)c(Cl)n3)C2)C1=O]

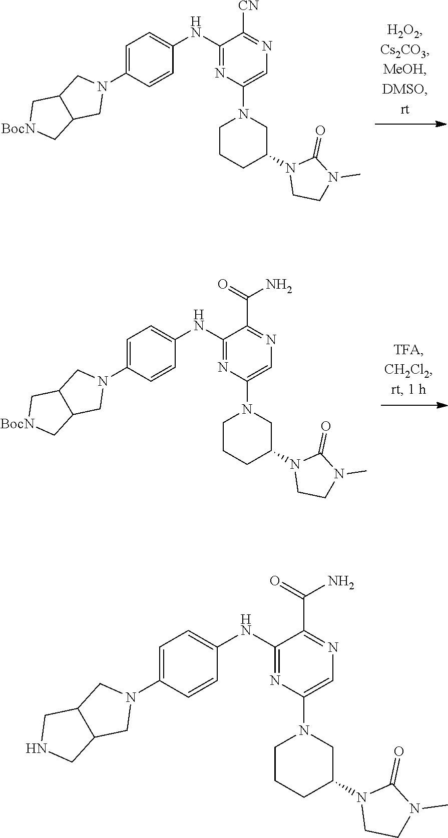 [SMILES: CN1CCN([C@@H]2CCCN(c3cnc(C#N)c(Nc4ccc(N5CC6CN(C(=O)OC(C)(C)C)CC6C5)cc4)n3)C2)C1=O ; CN1CCN([C@@H]2CCCN(c3cnc(C(N)=O)c(Nc4ccc(N5CC6CN(C(=O)OC(C)(C)C)CC6C5)cc4)n3)C2)C1=O ; CN1CCN([C@@H]2CCCN(c3cnc(C(N)=O)c(Nc4ccc(N5CC6CNCC6C5)cc4)n3)C2)C1=O]

Steps 1-5: Para-fluoronitrobenzene (1 eq) and amine (1 eq) were combined in DMF, followed by addition of potassium carbonate (2 eq). The reaction mixture was stirred at 90° C. for 3 h, then cooled to room temperature. The reaction mixture was then partitioned between ethyl acetate and water, and the organic layer was separated, dried over magnesium sulfate, and filtered. The crude material from was dissolved in ethanol and water (10:1). Ammonium chloride (3.5 eq) and iron (3 eq) were added, followed by vigorous stirring and heating to 90° C. for 5 h. The reaction was then filtered with Celite, and the Celite was further washed with ethyl acetate. The resulting solution was partitioned between ethyl acetate and water. The water layer was separated and re-extracted with ethyl acetate. The combined organic layers were washed with brine, dried over magnesium sulfate, and concentrated to provide tert-butyl 5-(4-aminophenyl)-hexahydropyrrolo[3,4-c]pyrrole-2-carboxylate (37% over 2 steps). LCMS C₁₇H₂₅N₃O₂ requires: 303.4, found: m/z=304.3 [M+H]⁺. tert-butyl 5-(4-aminophenyl)-hexahydropyrrolo[3,4-c]pyrrole-2-carboxylate was carried forward to 3-((4-(hexahydropyrrolo[3,4-c]pyrrol-2(1H)-yl)phenyl)amino)-5-((R)-3-(3-methyl-2-oxoimidazolidin-1-yl)piperidin-1-yl)pyrazine-2-carboxamide in a similar fashion as 5-[(3R)-3-(3-methyl-2-oxoimidazolidin-1-yl)piperidin-1-yl]-3-{[5-(piperazin-1-yl)pyridin-2-yl]amino}pyrazine-2-carboxamide.

### Example 52: Synthesis of 3-[(4-{1-[(1-{4-[1-(2,6-dioxopiperidin-3-yl)-4-methyl-5-oxo-1,2,4-triazol-3-yl]phenyl}azetidin-3-yl)methyl]piperidin-4-yl}phenyl)amino]-5-[(3R)-3-(3-methyl-2-oxoimidazolidin-1-yl)piperidin-1-yl]pyrazine-2-carboxamide

To a mixture of 5-[(3R)-3-(3-methyl-2-oxoimidazolidin-1-yl)piperidin-1-yl]-3-{[4-(piperidin-4-yl)phenyl]amino}pyrazine-2-carboxamide (60 mg, 0.13 mmol) in DCE (1.00 mL) was added N,N-diisopropylethylamine (0.27 mL, 1.5 mmol), 1-{4-[1-(2,6-dioxopiperidin-3-yl)-4-methyl-5-oxo-1,2,4-triazol-3-yl]phenyl}azetidine-3-carbaldehyde (55 mg, 0.15 mmol), and sodium triacetoxyborohydride (80 mg, 0.38 mmol). After 90 minutes, water was added and the mixture was extracted twice with DCM. The combined organic layers were concentrated then purified by preparative TLC eluted with 10% MeOH/DCM to provide 3-[(4-{1-[(1-{4-[1-(2,6-dioxopiperidin-3-yl)-4-methyl-5-oxo-1,2,4-triazol-3-yl]phenyl}azetidin-3-yl)methyl]piperidin-4-yl}phenyl)amino]-5-[(3R)-3-(3-methyl-2-oxoimidazolidin-1-yl)piperidin-1-yl]pyrazine-2-carboxamide (0.034 g, 29%).

### Example 53: Synthesis of 3-[(4-{1-[(1-{4-[1-(2,6-dioxopiperidin-3-yl)-4-methyl-5-oxo-1,2,4-triazol-3-yl]phenyl}azetidin-3-yl)methyl]piperidin-4-yl}phenyl)amino]-5-[(3R)-3-(2-oxopyrrolidin-1-yl)piperidin-1-yl]pyrazine-2-carboxamide

To a mixture of 5-[(3R)-3-(2-oxopyrrolidin-1-yl)piperidin-1-yl]-3-{[4-(piperidin-4-yl)phenyl]amino}pyrazine-2-carboxamide (16.2 mg, 0.04 mmol) in DCE (1.00 mL) was added N,N-diisopropylethylamine (0.06 mL, 0.42 mmol). 1-{4-[1-(2,6-dioxopiperidin-3-yl)-4-methyl-5-oxo-1,2,4-triazol-3-yl]phenyl}azetidine-3-carbaldehyde (12.9 mg, 0.04 mmol) was added followed by sodium triacetoxyborohydride (22 mg, 0.10 mmol). After 90 minutes, water was added and the mixture was extracted twice with DCM. The combined organic layers were purified by prep TLC eluted with 10% MeOH/DCM to provide 3-[(4-{1-[(1-{4-[1-(2,6-dioxopiperidin-3-yl)-4-methyl-5-oxo-1,2,4-triazol-3-yl]phenyl}azetidin-3-yl)methyl]piperidin-4-yl}phenyl)amino]-5-[(3R)-3-(2-oxopyrrolidin-1-yl)piperidin-1-yl]pyrazine-2-carboxamide (0.0094 g, 31%).

### Example 54: Synthesis of 2-(2,6-dioxopiperidin-3-yl)-5-(4-{[4-(4-{[4-methyl-3-oxo-6-(1,3-thiazol-2-yl)pyrazin-2-yl]amino}Phenyl)piperidin-1-yl]methyl}piperidin-1-yl)isoindole-1,3-dione

To a mixture of 1-methyl-3-{[4-(piperidin-4-yl)phenyl]amino}-5-(1,3-thiazol-2-yl)pyrazin-2-one (24 mg, 0.065 mmol) and 1-[2-(2,6-dioxopiperidin-3-yl)-1,3-dioxoisoindol-5-yl]piperidine-4-carbaldehyde (24 mg, 0.065 mmol) in 1,2-dichloroethane (1.0 mL) was added sodium triacetoxyborohydride (41 mg, 0.20 mmol). After 30 minutes, additional portions of 1-[2-(2,6-dioxopiperidin-3-yl)-1,3-dioxoisoindol-5-yl]piperidine-4-carbaldehyde (24 mg, 0.065 mmol) and sodium triacetoxyborohydride (41 mg, 0.20 mmol) were added. After 30 more minutes, water was added and the mixture was extracted twice with dichloromethane. The combined organic layers were concentrated then purified by preparative TLC eluted with 10% MeOH/DCM to provide 2-(2,6-dioxopiperidin-3-yl)-5-(4-{[4-(4-{[4-methyl-3-oxo-6-(1,3-thiazol-2-yl)pyrazin-2-yl]amino}phenyl)piperidin-1-yl]methyl}piperidin-1-yl)isoindole-1,3-dione (0.017 g, 35%).

### Example 55: Synthesis of (R)-3-((4-(3-aminopropoxy)phenyl)amino)-5-(3-(3-methyl-2-oxoimidazolidin-1-yl)piperidin-1-yl)pyrazine-2-carbonitrile

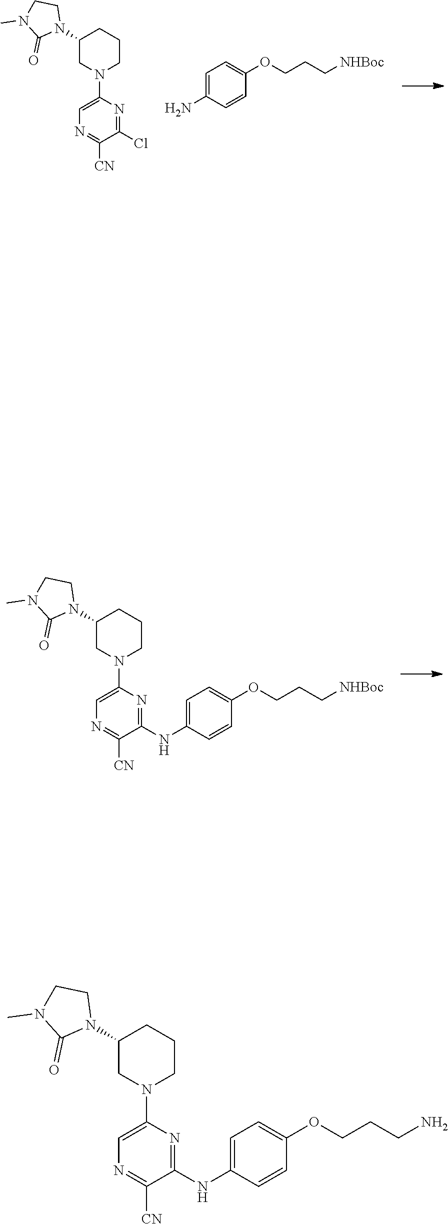 [SMILES: CN1CCN([C@@H]2CCCN(c3cnc(C#N)c(Cl)n3)C2)C1=O ; CC(C)(C)OC(=O)NCCCOc1ccc(N)cc1 ; CN1CCN([C@@H]2CCCN(c3cnc(C#N)c(Nc4ccc(OCCCNC(=O)OC(C)(C)C)cc4)n3)C2)C1=O ; CN1CCN([C@@H]2CCCN(c3cnc(C#N)c(Nc4ccc(OCCCN)cc4)n3)C2)C1=O]

Step 1. (R)-3-chloro-5-(3-(3-methyl-2-oxoimidazolidin-1-yl)piperidin-1-yl)pyrazine-2-carbonitrile, the aniline compound, Pd(OAc)₂ (0.15 eq), BINAP (0.15 eq), and cesium carbonate (2 eq) were combined in a microwave tube, followed by addition of dioxane (0.25 M). Nitrogen was bubbled through for 30 seconds, followed by capping. Heating to 90° C., followed by maintaining that temperature for 3 h provided a dark reaction mixture which was monitored by LCMS. The reaction was then cooled, and filtered through Celite, washing with ethyl acetate/methanol. The crude material was loaded onto silica and chromatographed (silica, 0-10% methanol in DCM), to provide the desired intermediate compound in 52% yield. Obtained tert-butyl (R)-(3-(4-((3-cyano-6-(3-(3-methyl-2-oxoimidazolidin-1-yl)piperidin-1-yl)pyrazin-2-yl)amino)phenoxy)propyl)carbamate. LCMS C₂₈H₃₈N₈O₄ requires: 550, found: m/z=551.7 [M+H]⁺.

Step 2. The intermediate from step 1 was dissolved in DCM:TFA (5:1 ratio, 0.2M) and the reaction was stirred for 4 h. The reaction mixture was concentrated by rotary evaporator, followed by chromatography (0-20% methanol in DCM) to provide desired amine in 92% yield. Obtained (R)-3-((4-(3-aminopropoxy)phenyl)amino)-5-(3-(3-methyl-2-oxoimidazolidin-1-yl)piperidin-1-yl)pyrazine-2-carbonitrile. LCMS C₂₃H₃₀N₈O₂ requires: 450.6, found: m/z=451.6 [M+H]⁺.

### Example 56: Synthesis of (R)-4-((3-carbamoyl-6-(3-(3-methyl-2-oxoimidazolidin-1-yl)piperidin-1-yl)pyrazin-2-yl)amino)benzoic acid

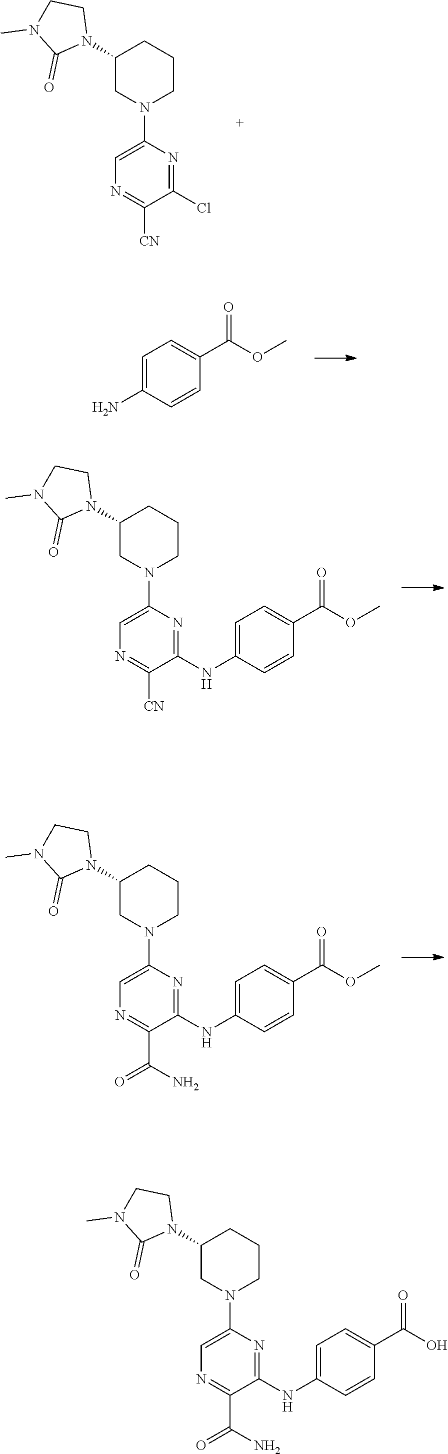

Step 1. (R)-3-chloro-5-(3-(3-methyl-2-oxoimidazolidin-1-yl)piperidin-1-yl)pyrazine-2-carbonitrile, the aniline compound, Pd(OAc)₂ (0.15 eq), BINAP (0.15 eq), and cesium carbonate (2 eq) were combined in a microwave tube, followed by addition of dioxane (0.25 M). Nitrogen was bubbled through for 30 seconds, followed by capping. Heating to 90° C., followed by maintaining that temperature for 3 h provided a dark reaction mixture which was monitored by LCMS. The reaction was then cooled, and filtered through Celite, washing with ethyl acetate/methanol. The crude material was loaded onto silica and chromatographed (silica, 0-10% methanol in DCM), to provide the desired intermediate compound in 74% yield. Obtained methyl (R)-4-((3-cyano-6-(3-(3-methyl-2-oxoimidazolidin-1-yl)piperidin-1-yl)pyrazin-2-yl)amino)benzoate. LCMS C₂₂H₂₅N₇O₃ requires: 435.5, found: m/z=436.6 [M+H]⁺.

Step 2. This material was then dissolved in methanol/DMSO (10:1) and a pellet of NaOH was added. The reaction was stirred for 5 minutes, followed by addition of 35% peroxide solution (2 mL of solution per mmol of reactant). This reaction mixture was stirred for 3 h, then partitioned between ethyl acetate and water. The organic layer was separated, and dried over magnesium sulfate. Chromatography (0-10% methanol in DCM) provided desired product in 48% yield. Obtained methyl (R)-4-((3-carbamoyl-6-(3-(3-methyl-2-oxoimidazolidin-1-yl)piperidin-1-yl)pyrazin-2-yl)amino)benzoate. LCMS C₂₂H₂₇N₇O₄ requires: 453.5, found: m/z=454.6 [M+H]⁺.

Step 3. Starting material was dissolved in THE (0.1 M) followed by addition of 2N LiGH (aq, 25% by volume of TIF). The reaction was stirred at 80° C. for 4 h. The reaction was then poured into ethyl acetate/2N HCl in a separatory funnel. The organic layer was separated, and the aqueous layer was further extracted with methylene chloride/methanol (10%). Both organic layers were dried over magnesium sulfate and filtered, followed by concentration by rotory evaporator, to provide desired carboxylic acid in 88% with no further purification. Obtained (R)-4-((3-carbamoyl-6-(3-(3-methyl-2-oxoimidazolidin-1-yl)piperidin-1-yl)pyrazin-2-yl)amino)benzoic acid. LCMS C₂₁H₂₅N₇O₄ requires: 439.5, found: m/z=440.6 [M+H]⁺.

### Example 57: General strategy for ring attachment

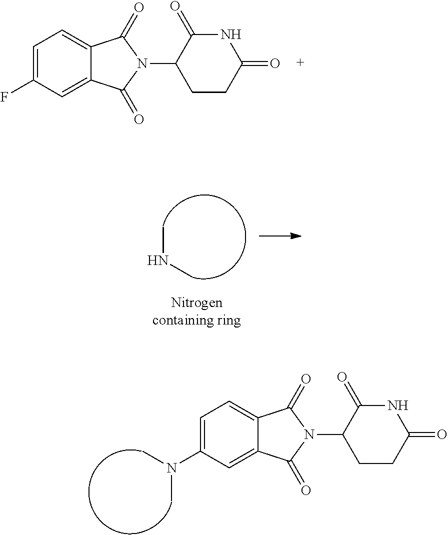

In a typical procedure, to a mixture of 2-(2,6-dioxopiperidin-3-yl)-5-fluoroisoindole-1,3-dione (˜1 eq.) and a cyclic compound containing a ring nitrogen (˜1 eq.) in a polar aprotic solvent such as N-methyl-2 pyrrolidinone (NMP), DMF, or DMSO, was added an organic base such as N,N-diisopropylethylamine (˜3 eq.). The resulting mixture was heated at a temperature ranging from about 50 to about 120° C. (e.g. 90° C.) for a time period of about 2 hours to about 24 hours (e.g. 16 hours). The mixture was diluted with an organic solvent such as ethyl acetate and washed with water. The organic layer was 1) dried using a simple procedure such as washing with brine and pouring over anhydrous Na₂SO₄, and then 2) concentrated in vacuo to provide a crude product that could be further purified using methods such as crystallization or flash chromatography.

### Example 57A: Synthesis of 2-(2,6-dioxopiperidin-3-yl)-5-(4-oxopiperidin-1-yl)isoindole-1,3-dione

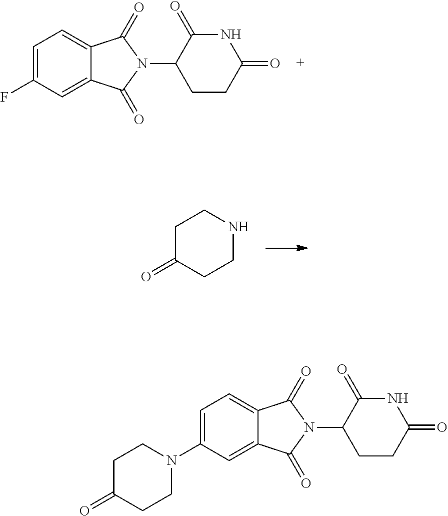 [SMILES: O=C1CCC(N2C(=O)c3ccc(F)cc3C2=O)C(=O)N1 ; O=C1CCNCC1 ; O=C1CCN(c2ccc3c(c2)C(=O)N(C2CCC(=O)NC2=O)C3=O)CC1]

To a mixture of 2-(2,6-dioxopiperidin-3-yl)-5-fluoroisoindole-1,3-dione (500 mg, 1.81 mmol) and 4-piperidinone hydrochloride (245 mg, 1.81 mmol) in 3 mL NMP was added N,N-diisopropylethylamine (703 mg, 5.43 mmol). The mixture was heated at 90° C. for 16 h. The mixture was diluted with ethyl acetate and washed with 2 portions of water. The organic layer was washed with brine, dried over anhydrous Na₂SO₄, and concentrated in vacuo. The crude residue was purified by flash chromatography eluted with 10 to 100% ethyl acetate/hexanes gradient to provide 2-(2,6-dioxopiperidin-3-yl)-5-(4-oxopiperidin-1-yl)isoindole-1,3-dione (131 mg, 20.4%). LCMS: C₁₈H₁₇N₃O₅ requires 355, found: m/z=356 [M+H]⁺.

### Example 58: Synthesis of 3-{[4-(1-{1-[2-(2,6-dioxopiperidin-3-yl)-1,3-dioxoisoindol-5-yl]piperidin-4-yl}azetidin-3-yl)phenyl]amino}-5-[(3R)-3-(3-methyl-2-oxoimidazolidin-1-yl)piperidin-1-yl]pyrazine-2-carboxamide

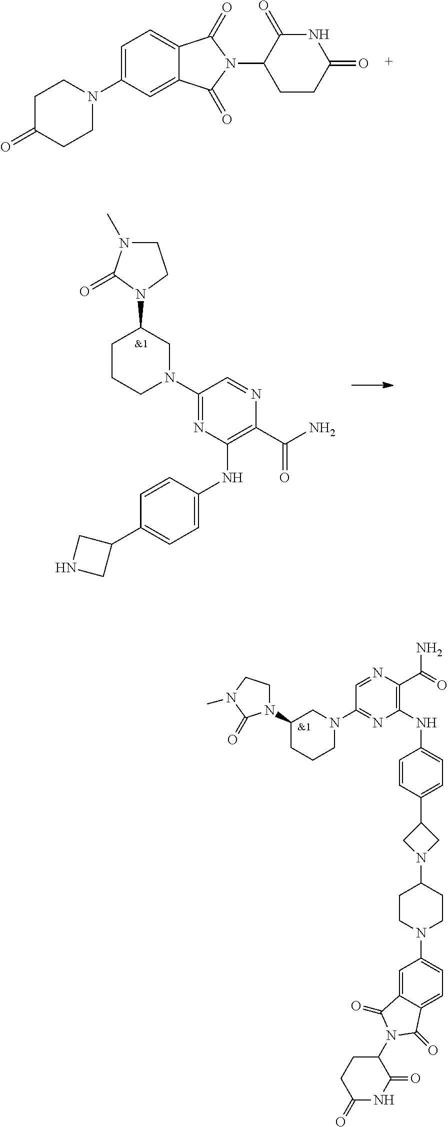 [SMILES: O=C1CCN(c2ccc3c(c2)C(=O)N(C2CCC(=O)NC2=O)C3=O)CC1 ; CN1CCN([C@@H]2CCCN(c3cnc(C(N)=O)c(Nc4ccc(C5CNC5)cc4)n3)C2)C1=O ; CN1CCN([C@@H]2CCCN(c3cnc(C(N)=O)c(Nc4ccc(C5CN(C6CCN(c7ccc8c(c7)C(=O)N(C7CCC(=O)NC7=O)C8=O)CC6)C5)cc4)n3)C2)C1=O]

Followed general procedure 2 starting from 3-{[4-(azetidin-3-yl)phenyl]amino}-5-[(3R)-3-(3-methyl-2-oxoimidazolidin-1-yl)piperidin-1-yl]pyrazine-2-carboxamide (55.0 mg, 0.12 mmol) and 2-(2,6-dioxopiperidin-3-yl)-5-(4-oxopiperidin-1-yl)isoindole-1,3-dione (43.4 mg, 0.12 mmol) to afford 3-{[4-(1-{1-[2-(2,6-dioxopiperidin-3-yl)-1,3-dioxoisoindol-5-yl]piperidin-4-yl}azetidin-3-yl)phenyl]amino}-5-[(3R)-3-(3-methyl-2-oxoimidazolidin-1-yl)piperidin-1-yl]pyrazine-2-carboxamide (9.50 mg, 9.9%). ¹H NMR (500 MHz, DMSO-d₆) δ 11.26 (s, 1H), 11.08 (s, 1H), 7.77 (d, J=2.9 Hz, 1H), 7.70-7.64 (m, 2H), 7.55 (d, J=8.3 Hz, 2H), 7.37-7.29 (m, 2H), 7.30-7.23 (m, 3H), 5.08 (dd, J=12.8, 5.4 Hz, 1H), 4.40 (d, J=12.6 Hz, 1H), 4.30 (d, J=13.6 Hz, 1H), 3.88 (dt, J=13.5, 4.5 Hz, 2H), 3.67-3.49 (m, 4H), 3.41-3.22 (m, 2H), 3.16 (ddd, J=13.0, 9.8, 3.1 Hz, 2H), 3.10-2.83 (m, 5H), 2.72 (s, 3H), 2.65-2.53 (m, 2H), 2.42-2.33 (m, 1H), 2.08-1.96 (m, 1H), 1.86-1.71 (m, 6H), 1.60-1.50 (m, 1H), 1.33-1.22 (m, 3H). LCMS: C₄₁H₄₇N₁₁O₆ requires 789, found: m/z=790 [M+H]⁺.

### Example 59: Synthesis of (R)-3-((4-(2,8-diazaspiro[4.5]decan-8-yl)phenyl)amino)-5-(3-(3-methyl-2-oxoimidazolidin-1-yl)piperidin-1-yl)pyrazine-2-carboxamide

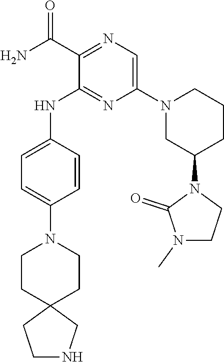 [SMILES: CN1CCN([C@@H]2CCCN(c3cnc(C(N)=O)c(Nc4ccc(N5CCC6(CCNC6)CC5)cc4)n3)C2)C1=O]

(R)-3-((4-(2,8-diazaspiro[4.5]decan-8-yl)phenyl)amino)-5-(3-(3-methyl-2-oxoimidazolidin-1-yl)piperidin-1-yl)pyrazine-2-carboxamide was made in an analogous fashion to (R)-3-((4-(2,9-diazaspiro[5.5]undecan-9-yl)phenyl)amino)-5-(3-(3-methyl-2-oxoimidazolidin-1-yl)piperidin-1-yl)pyrazine-2-carboxamide starting with tert-butyl 2,8-diazaspiro[4.5]decane-2-carboxylate (see Example 6). LCMS: C₂₈H₃₉N₉O₂ requires 519, found: m/z=520 [M+H]⁺.

### Example 60: Synthesis of 5-morpholino-3-[4-(4-piperidyl)anilino]pyrazine-2-carboxamide

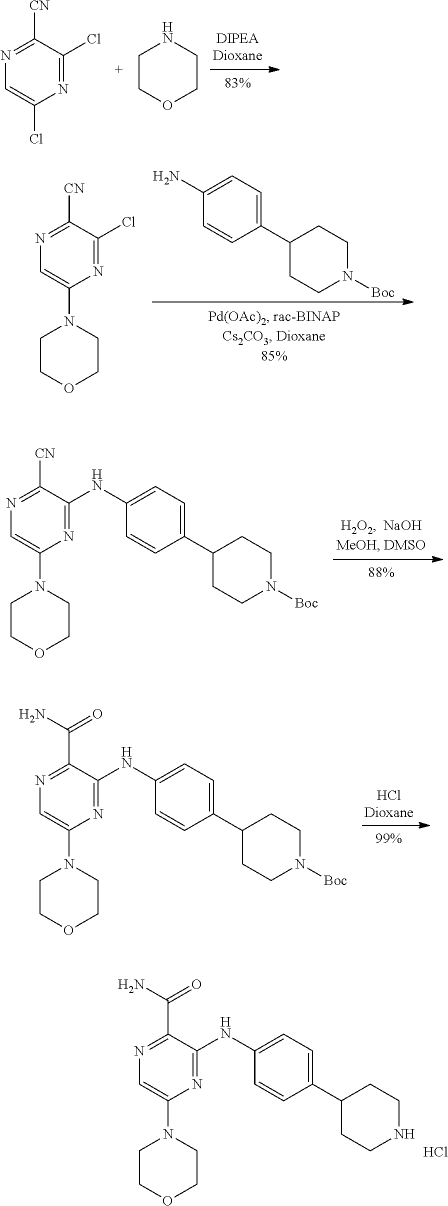

### Step 1: 3-chloro-5-morpholino-pyrazine-2-carbonitrile

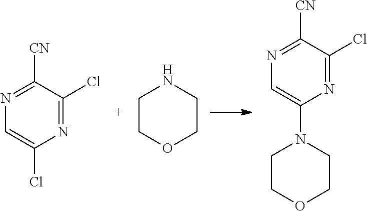 [SMILES: N#Cc1ncc(Cl)nc1Cl ; C1COCCN1 ; N#Cc1ncc(N2CCOCC2)nc1Cl]

To a solution of morpholine (2.65 mL, 30.4 mmol) and 3,5-dichloropyrazine-2-carbonitrile 1 (6.2 g, 35.5 mmol) in anhydrous DMF (40.0 mL) at rt, was added DIPEA (6.4 mL, 36.5 mmol). The reaction solution was stirred at rt for 4 h. The mixture was diluted with water (100 mL) and EtOAc (100 mL). The layers were separated, and the aqueous layer was extracted with EtOAc (3×20 mL). The combined organic layers were washed with water (2×20 mL) and brine (3×20 mL), then dried (Na₂SO₄), filtered and concentrated under reduced pressure to afford title compound (5.7 g, 83%) as a solid. MS (ESI) [M+H]⁺225.1.

### Step 2: Tert-butyl 4-[4-[(3-cyano-6-morpholino-pyrazin-2-yl)amino]phenyl]piperidine-1-carboxylate

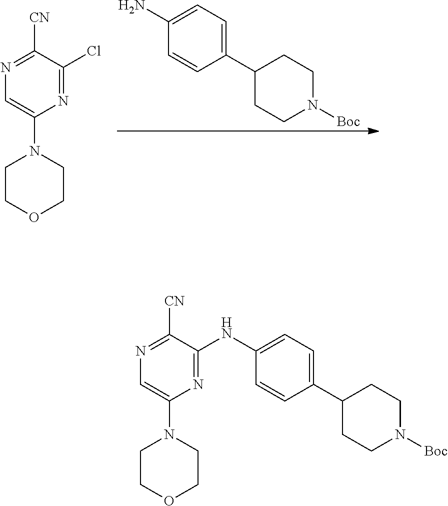 [SMILES: N#Cc1ncc(N2CCOCC2)nc1Cl ; CC(C)(C)OC(=O)N1CCC(c2ccc(N)cc2)CC1 ; CC(C)(C)OC(=O)N1CCC(c2ccc(Nc3nc(N4CCOCC4)cnc3C#N)cc2)CC1]

The mixture of 3-chloro-5-morpholino-pyrazine-2-carbonitrile (4.06 g, 18.1 mmol), tert-butyl 4-(4-aminophenyl)piperidine-1-carboxylate (5.0 g, 18.1 mmol), rac-BINAP (1.13 g, 1.81 mmol) and Cs₂CO₃ (17.7 g, 54.3 mmol) in anhydrous dioxane (60.0 mL) was degassed with N₂ for 10 min. Pd(OAc)₂ (406 mg, 1.81 mmol) was then added and the resulting mixture was heated at 80° C. for 16 h. The mixture was cooled to rt, the suspension was filtered on Celite and was washed with DCM (100 mL). The filtrate was concentrated under reduced pressure. The material was suspended in MeOH (50 mL) and sonicated for 2 min. The resulting solid filtered and dried to afford the title compound (7.1 g, 85%) as a solid. MS (ESI) [M-Boc+H]⁺ 365.3.

### Step 3: Tert-butyl 4-[4-[(3-carbamoyl-6-morpholino-pyrazin-2-yl)amino]phenyl]piperidine-1-carboxylate

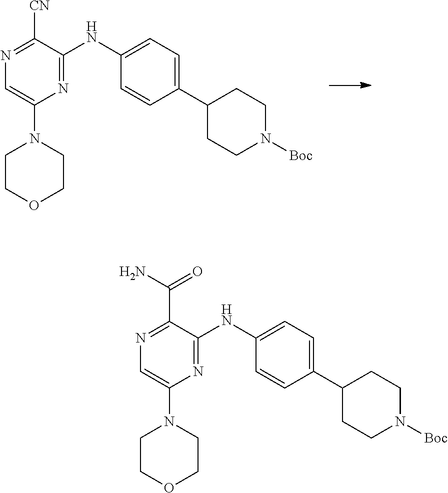 [SMILES: CC(C)(C)OC(=O)N1CCC(c2ccc(Nc3nc(N4CCOCC4)cnc3C#N)cc2)CC1 ; CC(C)(C)OC(=O)N1CCC(c2ccc(Nc3nc(N4CCOCC4)cnc3C(N)=O)cc2)CC1]

To a solution of tert-butyl 4-[4-[(3-cyano-6-morpholino-pyrazin-2-yl)amino]phenyl]piperidine-1-carboxylate (7.1 g, 15.3 mmol) in MeOH (100.0 mL) and DMSO (10.0 mL) at rt, was added NaOH (4 M in water, 7.64 mL 30.6 mmol) followed by H₂O₂(30% in water, 6.93 mL, 61.1 mmol). The reaction mixture was stirred at for 3.5 h. The mixture was diluted with acetonitrile (10 mL) and EtOAc (100 mL). The layers were separated, and the aqueous layer was extracted with EtOAc (3×50 mL). The combined organic layers were washed brine (2×30 mL), then dried (Na₂SO₄), filtered and concentrated under reduced pressure to afford the title compound as a solid (6.51 g, 88%). MS (ESI) [M−H]⁻ 481.4.

### Step 4: 5-morpholino-3-[4-(4-piperidyl)anilino]pyrazine-2-carboxamide hydrochloride

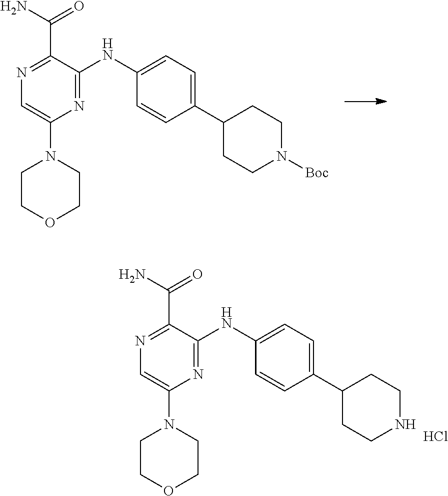 [SMILES: CC(C)(C)OC(=O)N1CCC(c2ccc(Nc3nc(N4CCOCC4)cnc3C(N)=O)cc2)CC1 ; NC(=O)c1ncc(N2CCOCC2)nc1Nc1ccc(C2CCNCC2)cc1 ; Cl]

To a solution of tert-butyl 4-[4-[(3-carbamoyl-6-morpholino-pyrazin-2-yl)amino]phenyl]piperidine-1-carboxylate (6.51 g, 13.5 mmol) in anhydrous DCM (20.0 mL) at rt, was added HCl (35.0 mL, 140 mmol, 4 M in dioxane) and the reaction mixture was stirred at rt for 2 h. The resulting solid was filtered, washed with acetonitrile (100 mL) and DCM (100 mL), then dried under reduced pressure to afford title compound (5.6 g, 99%) as a yellow solid. ¹H NMR (400 MHz, DMSO) δ 11.35 (s, 1H), 9.13-8.93 (m, 2H), 7.91-7.71 (m, 1H), 7.67 (s, 1H), 7.55 (d, J=8.6 Hz, 2H), 7.49-7.29 (m, 1H), 7.17 (d, J=8.6 Hz, 2H), 3.76-3.69 (m, 4H), 3.67-3.61 (m, 4H), 3.33 (d, J=12.7 Hz, 2H), 3.02-2.90 (m, 2H), 2.84-2.73 (m, 1H), 1.94-1.81 (m, 4H). MS (ESI) [M+H]⁺ 383.2.

### Example 61: 5-(4-methylpiperazin-1-yl)-3-[4-(4-piperidyl)anilino]pyrazine-2-carboxamide

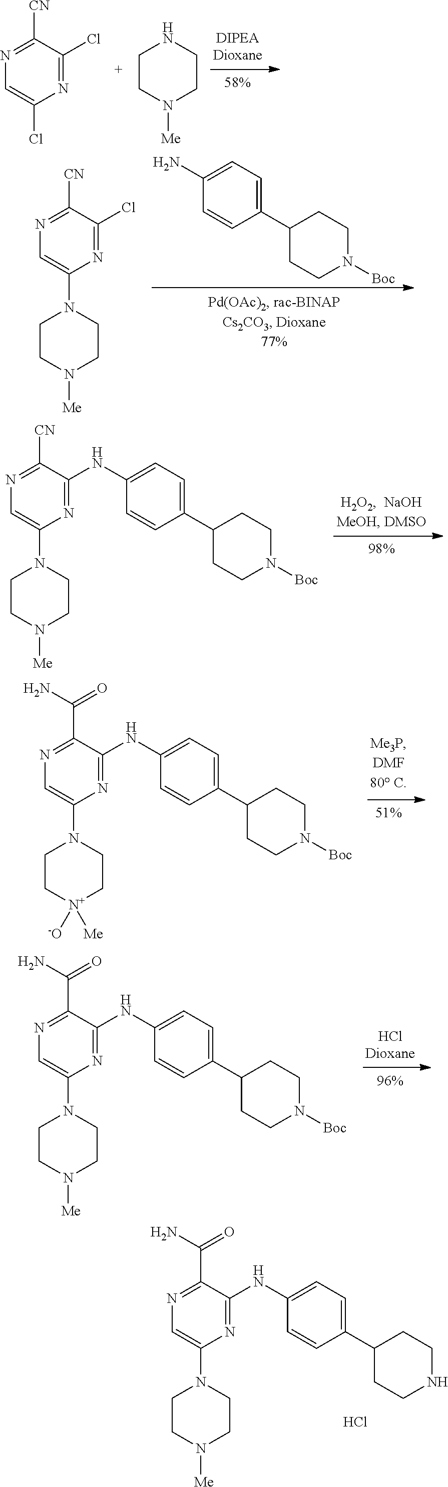

### Step 1: 3-chloro-5-(4-methylpiperazin-1-yl)pyrazine-2-carbonitrile

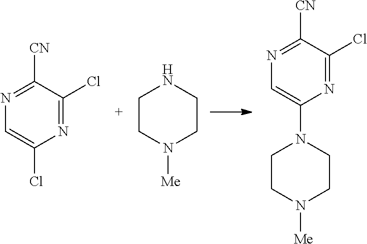 [SMILES: N#Cc1ncc(Cl)nc1Cl ; CN1CCNCC1 ; CN1CCN(c2cnc(C#N)c(Cl)n2)CC1]

To a solution of 1-methylpiperazine (3.3 mL, 30.0 mmol) and 3,5-dichloropyrazine-2-carbonitrile (6.1 g, 34.9 mmol) in anhydrous DMF (40 mL) at rt, was added DIPEA (6.26 mL, 35.9 mmol) and the reaction mixture was stirred at rt for 4 h. The mixture was diluted with water (100 mL) and EtOAc (100 mL). The layers were separated, and the aqueous layer was extracted with EtOAc (3×20 mL). The combined organic layers were washed with water (2×20 mL) and brine (3×20 mL), then dried (Na₂SO₄), filtered and concentrated under reduced pressure to afford the title compound (4.1 g, 58%) as a solid. MS (ESI) [M+H]⁺ 238.1.

### Step 2: Tert-butyl 4-[4-[[3-cyano-6-(4-methylpiperazin-1-yl)pyrazin-2-yl]amino]phenyl]piperidine-1-carboxylate

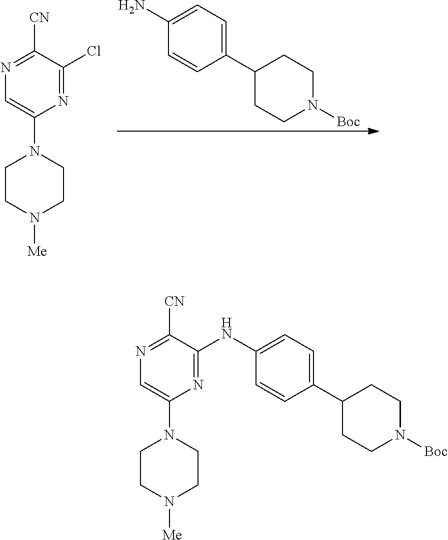 [SMILES: CN1CCN(c2cnc(C#N)c(Cl)n2)CC1 ; CC(C)(C)OC(=O)N1CCC(c2ccc(N)cc2)CC1 ; CN1CCN(c2cnc(C#N)c(Nc3ccc(C4CCN(C(=O)OC(C)(C)C)CC4)cc3)n2)CC1]

A mixture of 3-chloro-5-(4-methylpiperazin-1-yl)pyrazine-2-carbonitrile (4.1 g, 17.2 mmol), tert-butyl 1-(4-aminophenyl)piperidine-4-carboxylate (5.0 g, 18.1 mmol), rac-BINAP (1.13 g, 1.81 mmol), and Cs₂CO₃ (17.7 g, 54.3 mmol) in anhydrous dioxane (60.0 mL) was degassed with N₂ for 10 min. Pd(OAc)₂ (406 mg, 1.81 mmol) was then added and the resulting mixture was heated at 80° C. for 16 h. The mixture was cooled to rt, the suspension was filtered on Celite and was washed with DCM (100 mL). The filtrate was concentrated under reduced pressure. The material was suspended in MeOH (50 mL) and sonicated for 2 min. The resulting solid filtered and dried under reduced pressure to afford the title compound (6.68 g, 77%) as a solid. MS (ESI) [M-Boc+2H]⁺ 378.3.

### Step 3: Tert-butyl 4-[4-[[3-carbamoyl-6-(4-methyl-4-oxido-piperazin-4-ium-1-yl)pyrazin-2-yl]amino]phenyl]piperidine-1-carboxylate

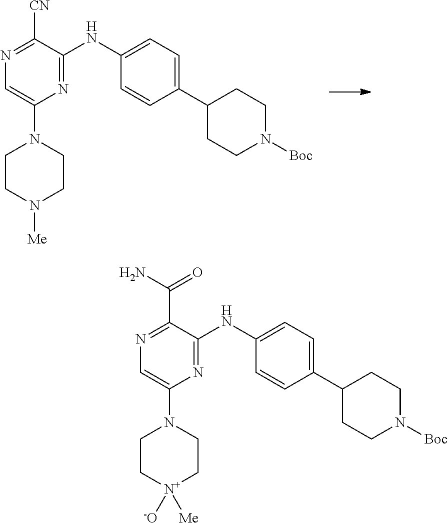

To a suspension of tert-butyl 4-[4-[[3-cyano-6-(4-methylpiperazin-1-yl)pyrazin-2-yl]amino]phenyl]piperidine-1-carboxylate (6.80 g, 14.2 mmol) in MeOH (100.0 mL) and DMSO (10.0 mL) at rt, was added aqueous NaOH (4 M in water, 7.1 mL 28.5 mmol), followed by H₂O₂(30% in water, 6.5 mL, 57.3 mmol) at rt. The reaction mixture was stirred at for 3.5 h. The mixture was diluted with cold water (50 mL). The resulting solid was filtered and washed with water (50 mL) and cold MeOH (40 mL) to afford the title compound (7.10 g, 98%) as a solid. ¹H NMR (400 MHz, DMSO) δ 11.30 (s, 1H), 7.82-7.73 (m, 1H), 7.67 (s, 1H), 7.51 (d, J=8.6 Hz, 2H), 7.41-7.31 (m, 1H), 7.18 (d, J=8.5 Hz, 2H), 4.13-3.98 (m, 2H), 3.73-3.58 (m, 4H), 2.90-2.71 (m, 2H), 2.69-2.57 (m, 1H), 2.46-2.35 (m, 4H), 2.22 (s, 3H), 1.79-1.67 (m, 2H), 1.52-1.43 (m, 2H), 1.42 (s, 9H). MS (ESI) [M−H]⁺ 510.5.

### Step 4: Tert-butyl 4-[4-[[3-carbamoyl-6-(4-methylpiperazin-1-yl)pyrazin-2-yl]amino]phenyl]piperidine-1-carboxylate

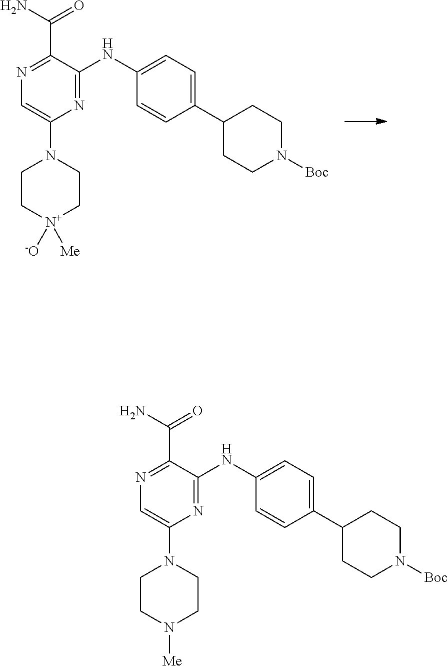

To solution of tert-butyl 4-[4-[[3-carbamoyl-6-(4-methyl-4-oxido-piperazin-4-ium-1-yl)pyrazin-2-yl]amino]phenyl]piperidine-1-carboxylate (4.50 g, 8.80 mmol) in anhydrous DMF (50.0 mL) at rt, was added trimethylphosphane (44.0 mL, 44.0 mmol, 1.0 M in THF) and the resulting mixture was heated at 80° C. for 4 h. The mixture was diluted with EtOAc (100 mL) and water (200 mL). The layers were separated, and the aqueous layer was extracted with EtOAc (3×50 mL). The combined organic layers were washed with brine (3×50 mL), then dried (Na₂SO₄), filtered and concentrated under reduced pressure to afford title compound (2.20 g, 51%) as a solid. MS (ESI) [M−H]⁻ 494.5.

### Step 5: 5-(4-methylpiperazin-1-yl)-3-[4-(4-piperidyl)anilino]pyrazine-2-carboxamide dihydrochloride

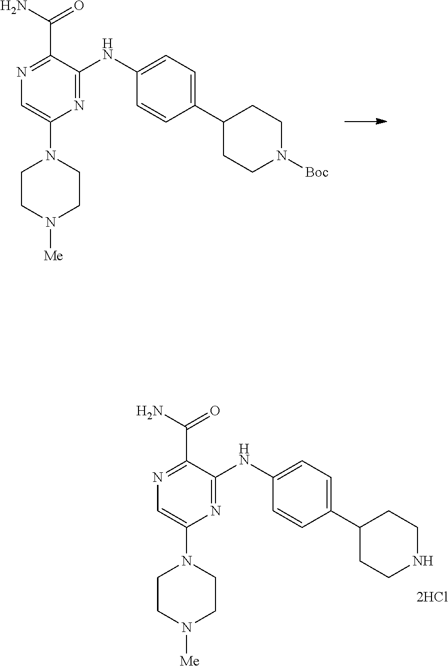 [SMILES: CN1CCN(c2cnc(C(N)=O)c(Nc3ccc(C4CCN(C(=O)OC(C)(C)C)CC4)cc3)n2)CC1 ; CN1CCN(c2cnc(C(N)=O)c(Nc3ccc(C4CCNCC4)cc3)n2)CC1]

To a solution of tert-butyl 4-[4-[[3-carbamoyl-6-(4-methylpiperazin-1-yl)pyrazin-2-yl]amino]phenyl]piperidine-1-carboxylate (2.60 g, 5.25 mmol) in anhydrous DCM (40.0 mL) at rt, was added HCl (15.0 mL, 60.0 mmol, 4.0 M in dioxane) and the resulting suspension was stirred at rt for 1 h. The resulting solid was filtered, washed with DCM (100 mL) and dried under reduced pressure to afford the title compound (2.35 g, 96%) as a yellow-orange solid. ¹H NMR (500 MHz, DMSO) δ 11.55-11.40 (m, 1H), 11.38 (s, 1H), 9.21-8.99 (m, 2H), 7.96-7.85 (m, 1H), 7.78 (s, 1H), 7.55 (d, J=8.4 Hz, 2H), 7.51-7.42 (m, 1H), 7.20 (d, J=8.4 Hz, 2H), 4.49 (d, J=14.3 Hz, 2H), 3.55-3.44 (m, 4H), 3.39-3.29 (m, 2H), 3.20-3.06 (m, 2H), 3.04-2.90 (m, 2H), 2.85-2.72 (m, 4H), 1.97-1.79 (m, 4H). MS (ESI) [M+H]⁺ 396.3.

### Example 62: Synthesis of 5-[4-(hydroxymethyl)-1-piperidyl]-3-[4-(4-piperidyl)anilino]pyrazine-2-carboxamide

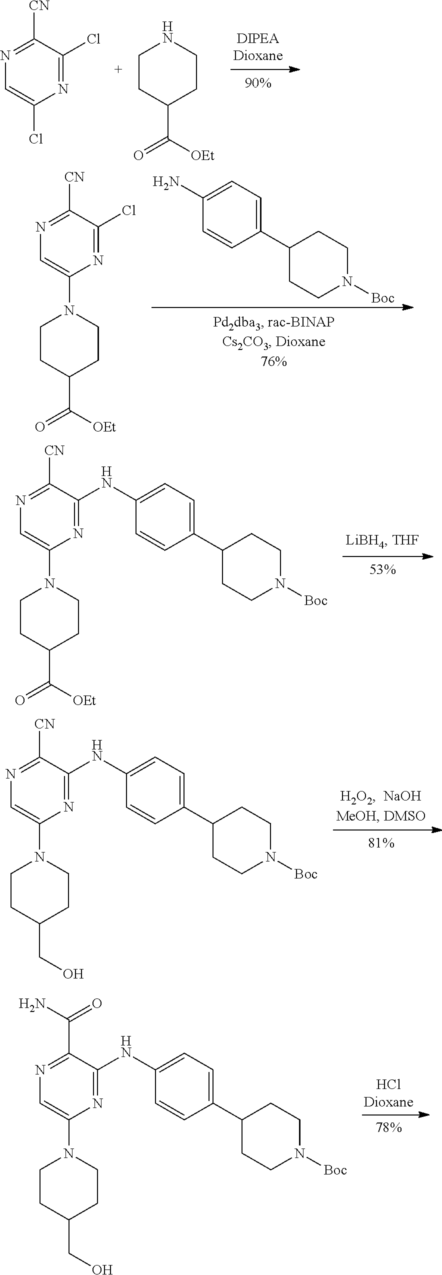

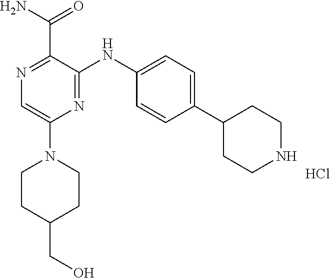 [SMILES: NC(=O)c1ncc(N2CCC(CO)CC2)nc1Nc1ccc(C2CCNCC2)cc1 ; Cl]

### Step 1: Ethyl 1-(6-chloro-5-cyano-pyrazin-2-yl)piperidine-4-carboxylate

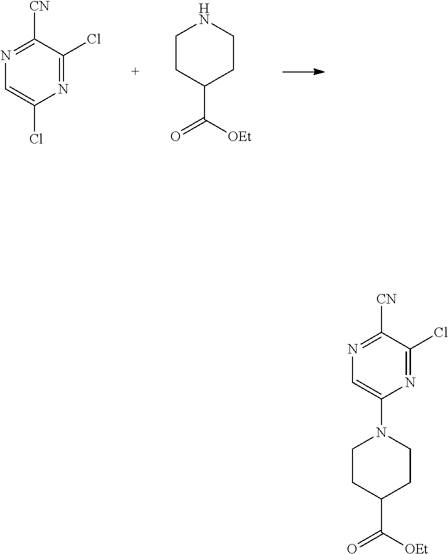 [SMILES: N#Cc1ncc(Cl)nc1Cl ; CCOC(=O)C1CCNCC1 ; CCOC(=O)C1CCN(c2cnc(C#N)c(Cl)n2)CC1]

To a solution of 3,5-dichloropyrazine-2-carbonitrile (6.96 g, 40.0 mmol) in anhydrous 1,4-dioxane (60.0 mL) at rt, was added ethyl piperidine-4-carboxylate (6.8 mL, 44 mmol) followed by DIPEA (14.0 mL, 80 mmol) and the reaction mixture was stirred for 1 h at rt. The volatiles were evaporated under reduced pressure. The material was purified by column chromatography on silica gel (dry loading, 220 g) using a gradient of 0-40% EtOAc in hexane to afford the title compound (11.8 g, 90%) as an oil. MS (ESI) [M+H]⁺ 295.2.

### Step 2: Tert-butyl 4-[4-[[3-cyano-6-(4-ethoxycarbonyl-1-piperidyl)pyrazin-2-yl]amino]phenyl]piperidine-1-carboxylate

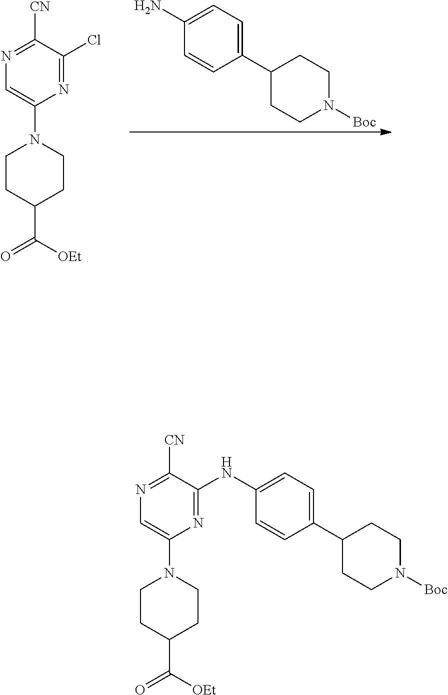 [SMILES: CCOC(=O)C1CCN(c2cnc(C#N)c(Cl)n2)CC1 ; CC(C)(C)OC(=O)N1CCC(c2ccc(N)cc2)CC1 ; CCOC(=O)C1CCN(c2cnc(C#N)c(Nc3ccc(C4CCN(C(=O)OC(C)(C)C)CC4)cc3)n2)CC1]

To a mixture of tert-butyl 4-(4-aminophenyl)piperidine-1-carboxylate (9.95 g, 36.0 mmol), Pd₂DBA₃ (1.65 g, 1.80 mmol), rac-BINAP (2.24 g, 3.60 mmol) and Cs₂CO₃ (29.3 g, 90.0 mmol) at rt, was added a degassed solution of ethyl 1-(6-chloro-5-cyano-pyrazin-2-yl)piperidine-4-carboxylate (11.8 g, 36.0 mmol) in anhydrous 1,4-dioxane (120.0 mL). The resulting mixture was further sparged with N₂ for 10 min and then stirred at 90° C. for 1.5 h, and was further stirred at 60° C. for 72 h. The mixture was cooled to rt, the suspension was filtered and washed with EtOAc. The filtrate was concentrated under reduced pressure, and the residue was diluted with MeOH (50 mL). The resulting suspension was filtered, and the collected solid was washed with MeOH (20 mL) to afford the title compound (9.1 g). The filtrate was concentrated under reduced pressure, then diluted with MeOH (25 mL). The resulting suspension was filtered, and the collected solid was washed with MeOH (10 mL) to afford the title compound (2.7 g). The filtrate was concentrated under reduced pressure and the material was purified by column chromatography on silica gel (dry loading, 120 g) using a gradient of 0-50% EtOAc in hexane to afford crude title compound. The material was diluted with MeOH (20 mL). The resulting suspension was filtered, and the collected solid was washed with MeOH (10 mL) to afford the title compound (2.9 g, total of 14.7 g, 76%) as a yellow solid. ¹H NMR (400 MHz, DMSO) δ 8.94 (s, 1H), 7.84 (s, 1H), 7.46 (d, J=8.6 Hz, 2H), 7.16 (d, J=8.7 Hz, 2H), 4.27-4.16 (m, 2H), 4.12-4.01 (m, 4H), 3.17-3.08 (m, 2H), 2.86-2.58 (m, 4H), 1.95-1.84 (m, 2H), 1.78-1.70 (m, 2H), 1.60-1.38 (m, 13H), 1.18 (t, J=7.1 Hz, 3H). MS (ESI) [M-Boc+2H]⁺ 435.4.

### Step 3: Tert-butyl 4-[4-[[3-cyano-6-[4-(hydroxymethyl)-1-piperidyl]pyrazin-2-yl]amino]phenyl]piperidine-1-carboxylate

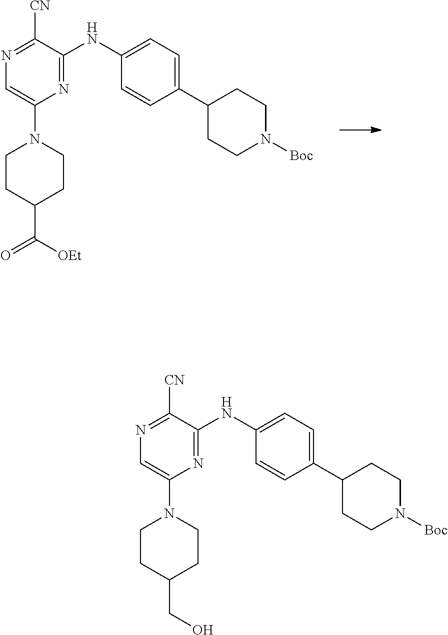 [SMILES: CCOC(=O)C1CCN(c2cnc(C#N)c(Nc3ccc(C4CCN(C(=O)OC(C)(C)C)CC4)cc3)n2)CC1 ; CC(C)(C)OC(=O)N1CCC(c2ccc(Nc3nc(N4CCC(CO)CC4)cnc3C#N)cc2)CC1]

To a solution of tert-butyl 4-[4-[[3-cyano-6-(4-ethoxycarbonyl-1-piperidyl)pyrazin-2-yl]amino]phenyl]piperidine-1-carboxylate (6.95 g, 13.0 mmol) in anhydrous THE (65 mL) at 0° C., was added LiBH₄ (2.0 M in THF, 13.0 mL, 26 mmol) and the resulting mixture was stirred at rt for 60 h. The mixture was diluted with EtOAc (65 mL) and saturated NH₄Cl (20 mL) and water (50 mL) [Note: caution: hydrogen evolution]. The layers were separated, and the aqueous phase was extracted with EtOAc (2×100 mL). The combined organic layers were washed with brine (50 mL), then dried (MgSO₄), filtered and concentrated under reduced pressure. The material was purified by column chromatography on silica gel (dry loading, 120 g) using a gradient of 0-65% EtOAc in hexane to afford the title compound (3.4 g, 53%) as a solid. ¹H NMR (500 MHz, DMSO) δ 8.90 (s, 1H), 7.83 (s, 1H), 7.47 (d, J=8.7 Hz, 2H), 7.15 (d, J=8.7 Hz, 2H), 4.48 (t, J=5.3 Hz, 1H), 4.34 (d, J=13.2 Hz, 2H), 4.12-4.01 (m, 2H), 3.26 (t, J=5.7 Hz, 2H), 2.94 (t, J=12.5 Hz, 2H), 2.78 (s, 2H), 2.63 (tt, J=12.3, 3.5 Hz, 1H), 1.77-1.65 (m, 5H), 1.51-1.43 (m, 2H), 1.41 (s, 9H), 1.11 (qd, J=12.0, 11.2, 3.4 Hz, 2H). MS (ESI) [M-Boc+2H]⁺ 393.3.

### Step 4: Tert-butyl 4-[4-[[3-carbamoyl-6-[4-(hydroxymethyl)-1-piperidyl]pyrazin-2-yl]amino]phenyl]piperidine-1-carboxylate

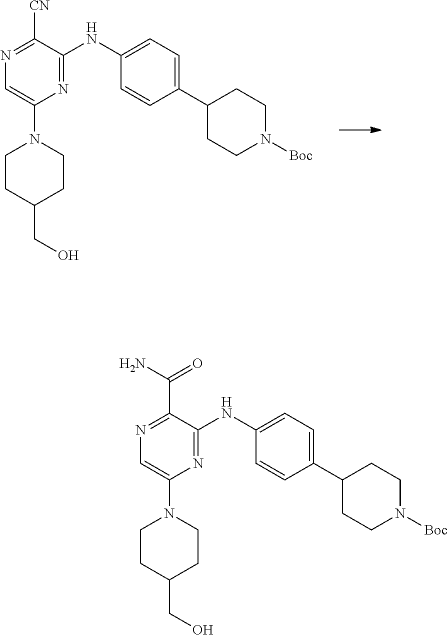 [SMILES: CC(C)(C)OC(=O)N1CCC(c2ccc(Nc3nc(N4CCC(CO)CC4)cnc3C#N)cc2)CC1 ; CC(C)(C)OC(=O)N1CCC(c2ccc(Nc3nc(N4CCC(CO)CC4)cnc3C(N)=O)cc2)CC1]

To a solution of tert-butyl 4-[4-[[3-cyano-6-[4-(hydroxymethyl)-1-piperidyl]pyrazin-2-yl]amino]phenyl]piperidine-1-carboxylate (3.40 g, 6.90 mmol) in MeOH (50.0 mL) and DMSO (5.0 mL) at rt, was added KOH (426 mg, 7.59 mmol) followed by 30% aqueous H₂O₂(0.85 mL, 8.3 mmol). The resulting mixture was stirred at rt for 4 h, and then cooled to 0° C. MeCN (2.0 mL) was added dropwise and the resulting mixture was then concentrated under reduced pressure. The residue was diluted with EtOAc (100 mL) and water (50 mL). The layers were separated, and the aqueous layer was extracted with EtOAc (2×50 mL). The combined organic layers were washed with water (50 mL) and brine (50 mL), then dried (MgSO₄), filtered and concentrated under reduced pressure to afford the title compound (2.84 g, 81%) as a solid. ¹H NMR (500 MHz, DMSO) δ 11.29 (s, 1H), 7.73 (d, J=3.2 Hz, 1H), 7.66 (s, 1H), 7.52 (d, J=8.7 Hz, 2H), 7.31 (d, J=3.0 Hz, 1H), 7.17 (d, J=8.7 Hz, 2H), 4.49 (t, J=5.4 Hz, 1H), 4.40 (d, J=13.1 Hz, 2H), 4.11-4.01 (m, 2H), 3.28 (t, J=5.8 Hz, 2H), 2.98 (td, J=13.0, 2.6 Hz, 2H), 2.90-2.67 (m, 2H), 2.62 (tt, J=11.7, 3.3 Hz, 1H), 1.80-1.66 (m, 5H), 1.51-1.43 (m, 2H), 1.41 (s, 9H), 1.20-1.12 (m, 2H). MS (ESI) [M−H]⁻ 509.5.

### Step 5: 5-[4-(hydroxymethyl)-1-piperidyl]-3-[4-(4-piperidyl)anilino]pyrazine-2-carboxamide; hydrochloride

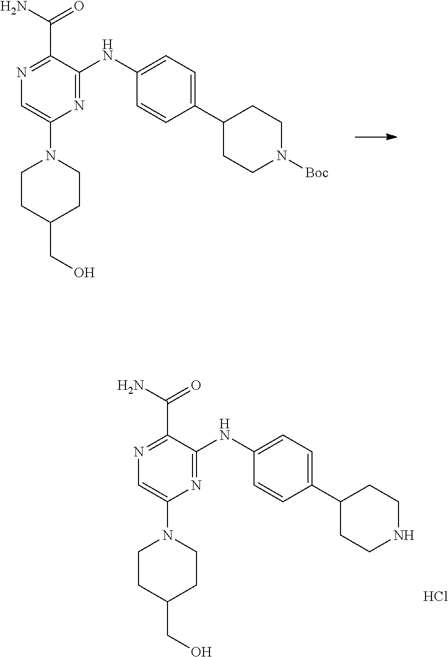 [SMILES: CC(C)(C)OC(=O)N1CCC(c2ccc(Nc3nc(N4CCC(CO)CC4)cnc3C(N)=O)cc2)CC1 ; NC(=O)c1ncc(N2CCC(CO)CC2)nc1Nc1ccc(C2CCNCC2)cc1 ; Cl]

To a solution of tert-butyl 4-[4-[[3-carbamoyl-6-[4-(hydroxymethyl)-1-piperidyl]pyrazin-2-yl]amino]phenyl]piperidine-1-carboxylate (2.84 g, 5.56 mmol) in anhydrous 1,4-dioxane (30 mL) at rt, HCl (4 M in dioxane, 11 mL, 44 mmol) was added, and the resulting mixture was stirred for 1 h at rt. Additional HCl (4 M in dioxane, 11 mL, 44 mmol) was added, and the resulting mixture was stirred for 4 h at rt. The resulting suspension was diluted with Et₂O (100 mL). The resulting solid was filtered and washed with MeCN (25 mL), DCM (25 mL) and Et₂O (25 mL). The solid was suspended in MeOH (50 mL) and stirred for 30 min and then Et₂O (100 mL) was added. The resulting suspension was filtered, and the solid was washed with Et₂O (25 mL) and the dried under reduced pressure to afford the title compound (2.19 g, 78%) of as a bright yellow solid. ¹H NMR (500 MHz, DMSO) δ 11.31 (s, 1H), 9.23-9.13 (m, 1H), 9.13-9.02 (m, 1H), 7.67 (s, 1H), 7.56 (d, J=8.7 Hz, 2H), 7.30 (s, 2H), 7.16 (d, J=8.7 Hz, 2H), 4.40 (d, J=13.2 Hz, 2H), 3.32 (d, J=12.5 Hz, 2H), 3.27 (d, J=6.1 Hz, 2H), 3.03-2.91 (m, 4H), 2.84-2.73 (m, 1H), 1.93-1.82 (m, 4H), 1.79-1.65 (m, 3H), 1.23-1.11 (m, 2H). *The —OH signal was not observed. MS (ESI) [M+H]⁺ 411.3.

### Example 63: Synthesis of 1-[5-carbamoyl-6-[4-(4-piperidyl)anilino]pyrazin-2-yl]piperidine-4-carboxylic acid

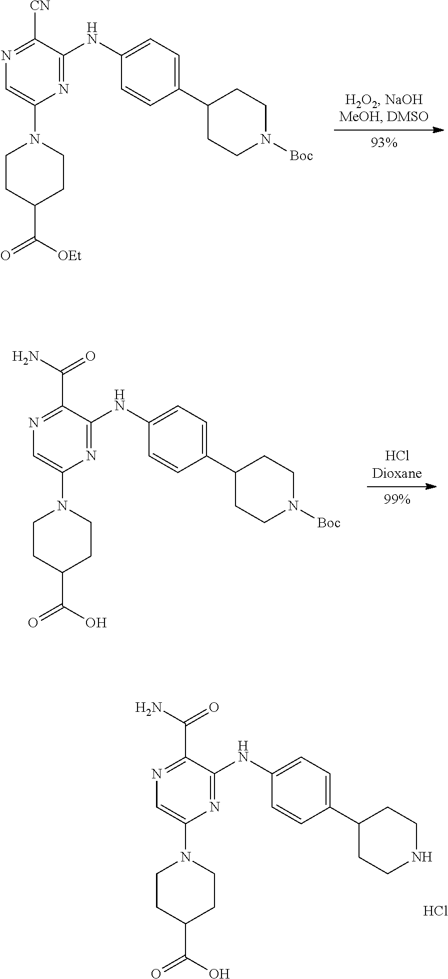 [SMILES: CCOC(=O)C1CCN(c2cnc(C#N)c(Nc3ccc(C4CCN(C(=O)OC(C)(C)C)CC4)cc3)n2)CC1 ; CC(C)(C)OC(=O)N1CCC(c2ccc(Nc3nc(N4CCC(C(=O)O)CC4)cnc3C(N)=O)cc2)CC1 ; NC(=O)c1ncc(N2CCC(C(=O)O)CC2)nc1Nc1ccc(C2CCNCC2)cc1 ; Cl]

### Step 1: 1-[6-[4-(1-tert-butoxycarbonyl-4-piperidyl)anilino]-5-carbamoyl-pyrazin-2-yl]piperidine-4-carboxylic acid

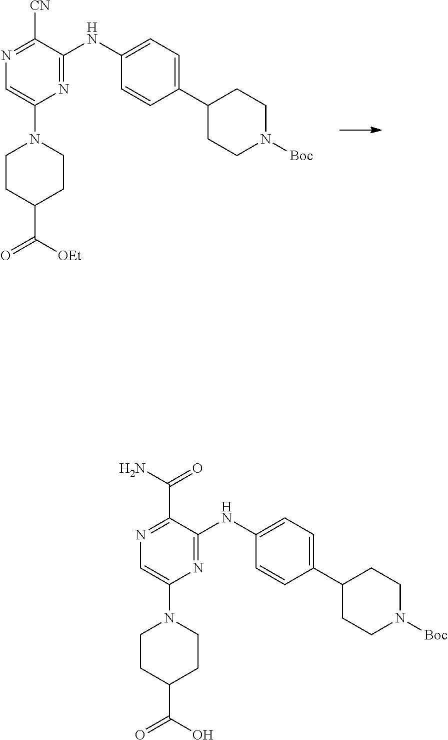 [SMILES: CCOC(=O)C1CCN(c2cnc(C#N)c(Nc3ccc(C4CCN(C(=O)OC(C)(C)C)CC4)cc3)n2)CC1 ; CC(C)(C)OC(=O)N1CCC(c2ccc(Nc3nc(N4CCC(C(=O)O)CC4)cnc3C(N)=O)cc2)CC1]

To a solution of tert-butyl 4-[4-[[3-cyano-6-(4-ethoxycarbonyl-1-piperidyl)pyrazin-2-yl]amino]phenyl]piperidine-1-carboxylate in MeOH (75.0 mL) and DMSO (5.0 mL) at rt, was added KOH (1.41 g, 25.1 mmol) followed by 30% aq. H₂O₂(1.3 mL, 13 mmol). The resulting mixture was stirred at rt 2 h, and then additional KOH (512 mg, 9.13 mmol) and 30% aq. H₂O₂(0.70 mL, 6.9 mmol) were added. The reaction mixture was stirred for 2 h at rt. The mixture was cooled to 0° C., and MeCN (2.0 mL) was added dropwise. The volatiles were evaporated under reduced pressure, and the residue was diluted with water (50 mL). 2 M aqueous NaHSO₄ (50 mL) was then added, and the resulting suspension was filtered. The collected solid was washed with water (3×50 mL) and dried under reduced pressure to afford title compound (5.56 g, 93%) as a solid. ¹H NMR (500 MHz, DMSO) δ 11.85 (s, 1H), 11.29 (s, 1H), 7.75 (d, J=2.5 Hz, 1H), 7.68 (s, 1H), 7.52 (d, J=8.7 Hz, 2H), 7.33 (d, J=2.8 Hz, 1H), 7.19 (d, J=8.5 Hz, 2H), 4.27 (d, J=13.7 Hz, 2H), 4.06 (d, J=9.8 Hz, 2H), 3.16 (t, J=10.9 Hz, 2H), 2.79 (s, 2H), 2.68-2.56 (m, 2H), 2.01-1.88 (m, 2H), 1.75 (d, J=12.5 Hz, 2H), 1.62-1.53 (m, 2H), 1.51-1.43 (m, 2H), 1.41 (s, 9H). MS (ESI) [M−H]⁻ 523.5.

### Step 2: 1-[5-carbamoyl-6-[4-(4-piperidyl)anilino]pyrazin-2-yl]piperidine-4-carboxylic acid hydrochloride

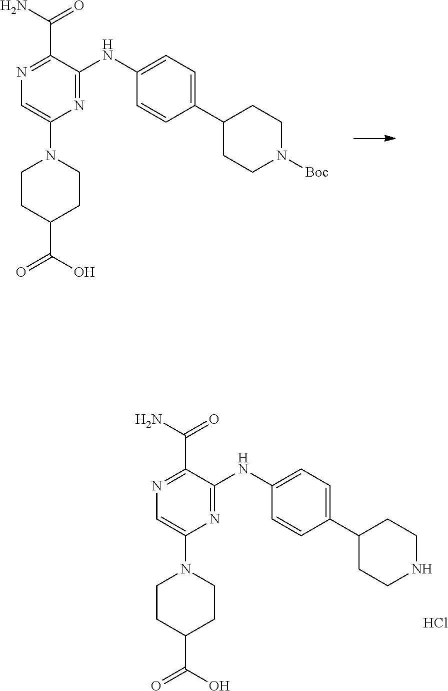 [SMILES: CC(C)(C)OC(=O)N1CCC(c2ccc(Nc3nc(N4CCC(C(=O)O)CC4)cnc3C(N)=O)cc2)CC1 ; NC(=O)c1ncc(N2CCC(C(=O)O)CC2)nc1Nc1ccc(C2CCNCC2)cc1 ; Cl]

To a solution of 1-[6-[4-(1-tert-butoxycarbonyl-4-piperidyl)anilino]-5-carbamoyl-pyrazin-2-yl]piperidine-4-carboxylic acid (5.56 g, 10.6 mmol) in anhydrous 1,4-dioxane (50.0 mL) at rt, was added HCl (4 M in dioxane, 21 mL, 84 mmol) and the resulting mixture was stirred for 1 h at rt. Additional HCl (4 M in dioxane, 21 mL, 84 mmol) was added, and the resulting mixture was further stirred for 4 h at rt. The resulting suspension was diluted with Et₂O (100 mL). The resulting solid was filtered and washed with MeCN (50 mL), DCM (50 mL) and Et₂O (50 mL). The solid was suspended in MeOH (100 mL) and stirred for 30 min and then Et₂O (100 mL) was added. The resulting suspension was filtered, and the solid was washed with Et₂O (50 mL) and then dried under reduced pressure to afford the title compound (5.17 g, 99%) as a bright yellow solid. ¹H NMR (500 MHz, DMSO) δ 11.32 (s, 1H), 9.19-9.09 (m, 1H), 9.00 (d, J=10.4 Hz, 1H), 7.85 (s, 1H), 7.69 (s, 1H), 7.55 (d, J=8.7 Hz, 2H), 7.34 (s, 1H), 7.18 (d, J=8.7 Hz, 2H), 4.27 (d, J=13.1 Hz, 2H), 3.32 (d, J=12.5 Hz, 2H), 3.21-3.12 (m, 2H), 3.02-2.90 (m, 2H), 2.84-2.74 (m, 1H), 2.65-2.56 (m, 1H), 1.99-1.78 (m, 6H), 1.64-1.53 (m, 2H). *The —COOH signal was not observed. MS (ESI) [M+H]⁺ 425.3.

### Example 64: Synthesis of 3-((4-(piperidin-4-yl)phenyl)amino)-5-(pyrrolidin-1-yl)pyrazine 2-carboxamide

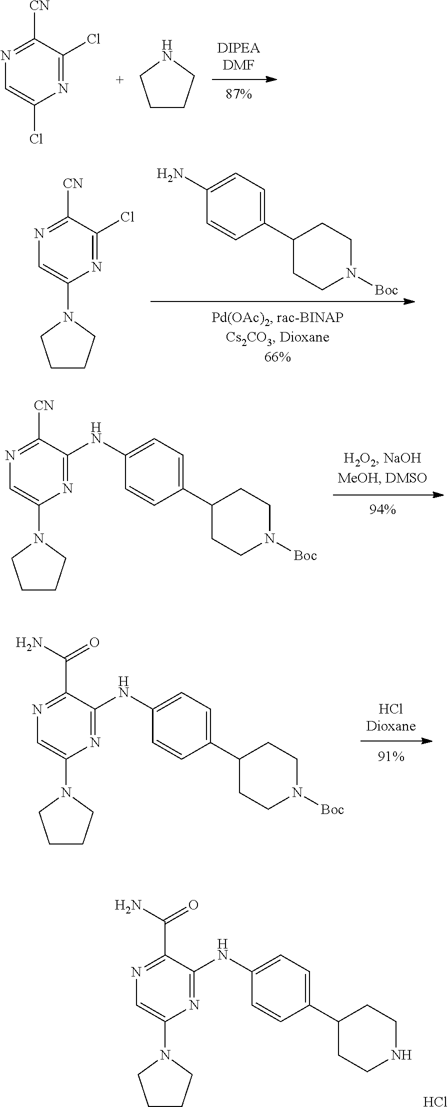

### Step 1: 3-chloro-5-pyrrolidin-1-vl-pyrazine-2-carbonitrile

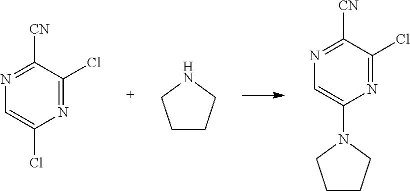 [SMILES: N#Cc1ncc(Cl)nc1Cl ; C1CCNC1 ; N#Cc1ncc(N2CCCC2)nc1Cl]

To a solution of pyrrolidine (1.52 mL, 18.2 mmol) and 3,5-dichloropyrazine-2-carbonitrile (3.17 g, 18.2 mmol) in anhydrous DMF (20.0 mL) at rt, was added DIPEA (3.81 mL, 21.9 mmol). The resulting solution was stirred at rt for 1 h. The mixture was diluted with water (100 mL) and the resulting solid was collected by filtration then dried under reduced pressure to afford title compound (3.3 g, 87%) as a solid. MS (ESI) [M+H]⁺ 209.1.

### Step 2: Tert-butyl 4-[4-[(3-cyano-6-pyrrolidin-1-yl-pyrazin-2-yl)amino]phenyl]piperidine-1-carboxylate

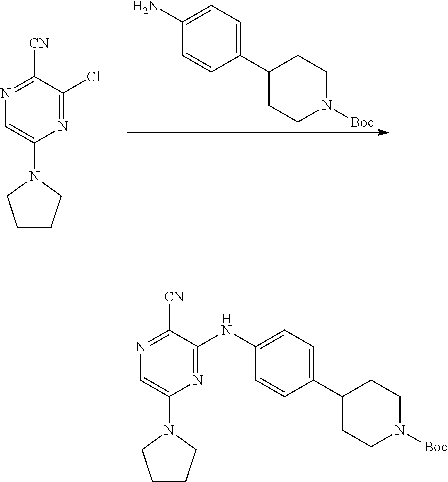 [SMILES: N#Cc1ncc(N2CCCC2)nc1Cl ; CC(C)(C)OC(=O)N1CCC(c2ccc(N)cc2)CC1 ; CC(C)(C)OC(=O)N1CCC(c2ccc(Nc3nc(N4CCCC4)cnc3C#N)cc2)CC1]

A mixture of 3-chloro-5-pyrrolidin-1-yl-pyrazine-2-carbonitrile (3.40 g, 16.3 mmol), tert-butyl 4-(4-aminophenyl)piperidine-1-carboxylate (4.50 g, 16.3 mmol), rac-BINAP (1.13 g, 1.81 mmol) and Cs₂CO₃ (15.9 g, 48.9 mmol) in anhydrous dioxane (60.0 mL) was degassed with N₂ for 10 min. Pd(OAc)₂ (406 mg, 1.81 mmol) was then added and the resulting mixture was heated at 80° C. for 16 h. The mixture was cooled to rt, the suspension was filtered on Celite and was washed with DCM (100 mL). The filtrate was concentrated reduced pressure. The material was suspended in MeOH (50 mL) and sonicated for 2 min. The resulting solid was filtered and dried under reduced pressure to afford the title compound (4.80 g, 66%) as a solid. ¹H NMR (400 MHz, DMSO) δ 8.83 (s, 1H), 7.59 (d, J=8.7 Hz, 2H), 7.50 (s, 1H), 7.15 (d, J=8.6 Hz, 2H), 4.13-3.96 (m, 2H), 3.52-3.42 (m, 4H), 2.88-2.70 (m, 2H), 2.67-2.58 (m, 1H), 2.01-1.84 (m, 4H), 1.79-1.68 (m, 2H), 1.52-1.42 (m, 2H), 1.41 (s, 9H). MS (ESI) [M-Boc+2H]⁺ 349.3.

### Step 3: Tert-butyl 4-[4-[(3-carbamoyl-6-pyrrolidin-1-yl-pyrazin-2-yl)amino]phenyl]piperidine-1-carboxylate

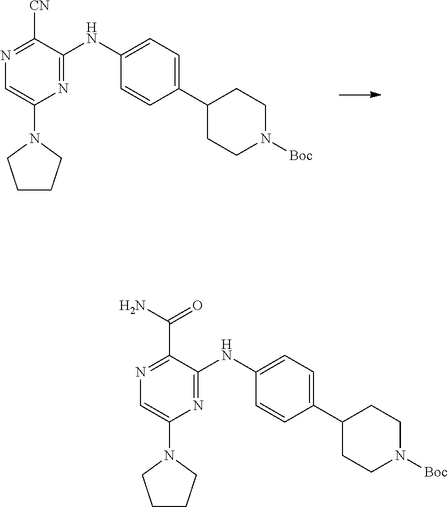 [SMILES: CC(C)(C)OC(=O)N1CCC(c2ccc(Nc3nc(N4CCCC4)cnc3C#N)cc2)CC1 ; CC(C)(C)OC(=O)N1CCC(c2ccc(Nc3nc(N4CCCC4)cnc3C(N)=O)cc2)CC1]

To a solution of tert-butyl 4-[4-[(3-cyano-6-pyrrolidin-1-yl-pyrazin-2-yl)amino]phenyl]piperidine-1-carboxylate (4.80 g, 10.7 mmol) in MeOH (100.0 mL) and DMSO (10.0 mL) at rt, was added NaOH (4 M in water, 5.35 mL 21.4 mmol) followed by H₂O₂(30% in water, 4.85 mL, 42.8 mmol) at rt. The reaction mixture was stirred at for 18 h, and then water (100 mL) was added. The resulting solid was filtered and dried under reduced pressure to afford the title compound (4.70 g, 94%) as a solid. ¹H NMR (500 MHz, DMSO) δ 11.40 (s, 1H), 7.75-7.71 (m, 1H), 7.64 (d, J=8.6 Hz, 2H), 7.35 (s, 1H), 7.31-7.25 (m, 1H), 7.18 (d, J=8.6 Hz, 2H), 4.15-4.01 (m, 2H), 3.59-3.49 (m, 4H), 2.95-2.70 (m, 2H), 2.67-2.59 (m, 1H), 2.04-1.93 (m, 4H), 1.79-1.72 (m, 2H), 1.51-1.44 (m, 2H), 1.42 (s, 9H).

### Step 4: 3-[4-(4-piperidyl)anilino]-5-pyrrolidin-1-yl-pyrazine-2-carboxamide hydrochloride

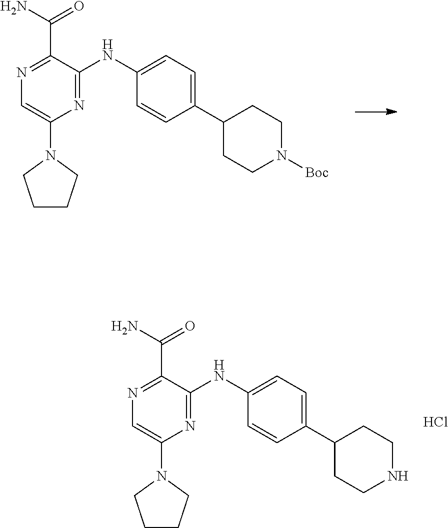 [SMILES: CC(C)(C)OC(=O)N1CCC(c2ccc(Nc3nc(N4CCCC4)cnc3C(N)=O)cc2)CC1 ; NC(=O)c1ncc(N2CCCC2)nc1Nc1ccc(C2CCNCC2)cc1 ; Cl]

To a solution of tert-butyl 4-[4-[(3-carbamoyl-6-pyrrolidin-1-yl-pyrazin-2-yl)amino]phenyl]piperidine-1-carboxylate (4.80 g, 10.3 mmol) in DCM (75 mL) and MeOH (25 mL) at rt, was added HCl (20.0 mL, 80.0 mmol, 4.0 M in dioxane) and the resulting mixture was stirred at rt for 2 h. The suspension was filtered, washed with DCM and then dried under reduced pressure to afford title compound (3.78 g, 91%) as yellow-orange solid. ¹H NMR (400 MHz, DMSO) δ 11.42 (s, 1H), 8.97-8.76 (m, 2H), 7.82-7.59 (m, 1H), 7.68 (d, J=8.5 Hz, 2H), 7.35 (s, 1H), 7.33-7.19 (m, 1H), 7.16 (d, J=8.5 Hz, 2H), 3.56-3.47 (m, 4H), 3.38-3.30 (m, 2H), 3.03-2.90 (m, 2H), 2.83-2.73 (m, 1H), 2.03-1.96 (m, 4H), 1.92-1.77 (m, 4H). MS (ESI) [M+H]⁺ 367.2.

### Example 65: Synthesis of (S)-1-(4-(4-methylthiazol-5-yl)phenyl)ethan-1-amine hydrogen chloride [Intermediate 1]

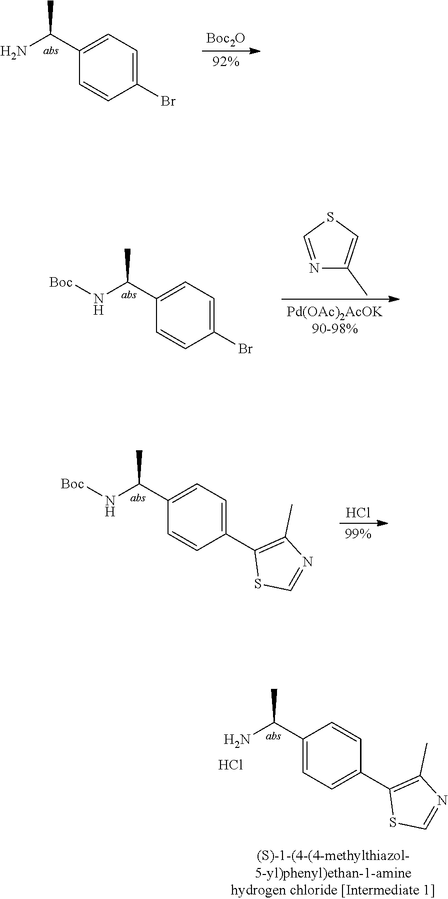

### Step 1: Tert-butyl N-[(1S)-1-(4-bromophenyl)ethyl]carbamate

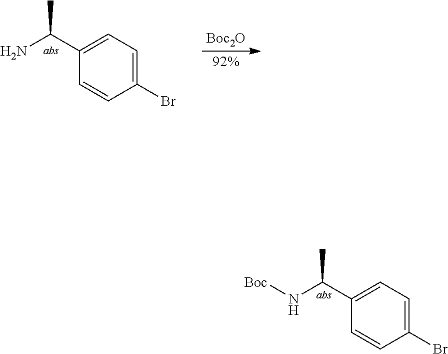

To a solution of (1S)-1-(4-bromophenyl)ethanamine (25.0 g, 125 mmol) and BOC anhydride (32.7 g, 150 mmol) in DCM (250 mL) at 0° C., TEA (34.8 mL, 250 mmol) was added. The reaction mixture was stirred at 0° C. for 15 min and then 18 h at rt. The mixture was diluted with water (250 mL) and the layers were separated. The aqueous layer was extracted with EtOAc (2×100 mL). The combined organic layers were washed with brine (100 mL) then dried (Na₂SO₄), filtered, and concentrated under reduced pressure. The resulting solid was triturated with hexanes (400 mL), filtered and washed with hexanes (500 mL) to afford the title compound as a solid (34.5 g, 92%). MS (ESI) [M−tBu]⁺ 244.0, 246.0.

### Step 2: Tert-butyl N-[(1S)-1-[4-(4-methylthiazol-5-yl)phenyl]ethyl]carbamate

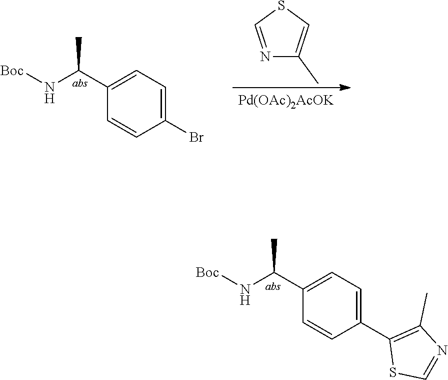

A mixture of tert-butyl N-[(1S)-1-(4-bromophenyl)ethyl]carbamate (15.0 g, 50.0 mmol), potassium acetate (9.81 g, 100 mmol) and Pd(OAc)₂ (112 mg, 0.50 mmol) in DMA (100 mL) at rt, was added 4-methylthiazole (9.10 mL, 100 mmol). The mixture was purged with nitrogen and put under vacuum (3×cycle) and then stirred at 120° C. for 2 h. The mixture was cooled to rt and diluted with water (250 mL). The resulting solid was filtered and washed with water (500 mL). The solid was dried in a vacuum oven at 65° C. for 18 h to afford the title compound 15.6 g, 98%). MS (ESI) [M+H]⁺ 319.2.

### Step 3: (1S)-1-[4-(4-methylthiazol-5-yl)phenyl]ethanamine hydrochloride [Intermediate 1]

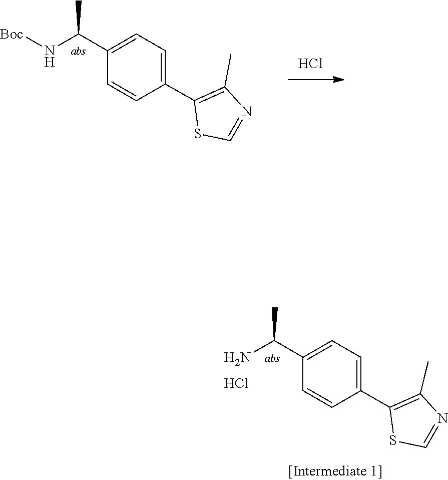 [SMILES: Cc1ncsc1-c1ccc([C@H](C)NC(=O)OC(C)(C)C)cc1 ; Cl ; Cl ; Cc1ncsc1-c1ccc([C@H](C)N)cc1]

To a solution of tert-butyl N-[(1S)-1-[4-(4-methylthiazol-5-yl)phenyl]ethyl]carbamate (17.4 g, 54.6 mmol) in DCM (200 mL) at 0° C., was added HCl (4M in dioxane, 200 mL, 800 mmol) and the mixture was warmed to rt and stirred for 3 h. The mixture was diluted with ether (50 mL) and the resulting solid was filtered. The solid was washed with ether (500 mL) and dried to afford the title compound as a solid (15.0 g, quant). ¹H NMR (400 MHz, DMSO) δ 9.11 (s, 1H), 8.69 (br s, 3H), 7.67-7.62 (m, 2H), 7.58-7.53 (m, 2H), 4.44 (dt, J=11.9, 5.9 Hz, 1H), 2.47 (s, 3H), 1.55 (d, J=6.8 Hz, 3H). MS (ESI) [M+H]⁺ 202.2.

### Example 66: Synthesis of (2S,4R)-1-((S)-2-amino-3,3-dimethylbutanoyl)-4-hydroxy-N—((S)-1-(4-(4-methylthiazol-5-yl)phenyl)ethyl)pyrrolidine-2-carboxamide hydrochloride [Intermediate 2]

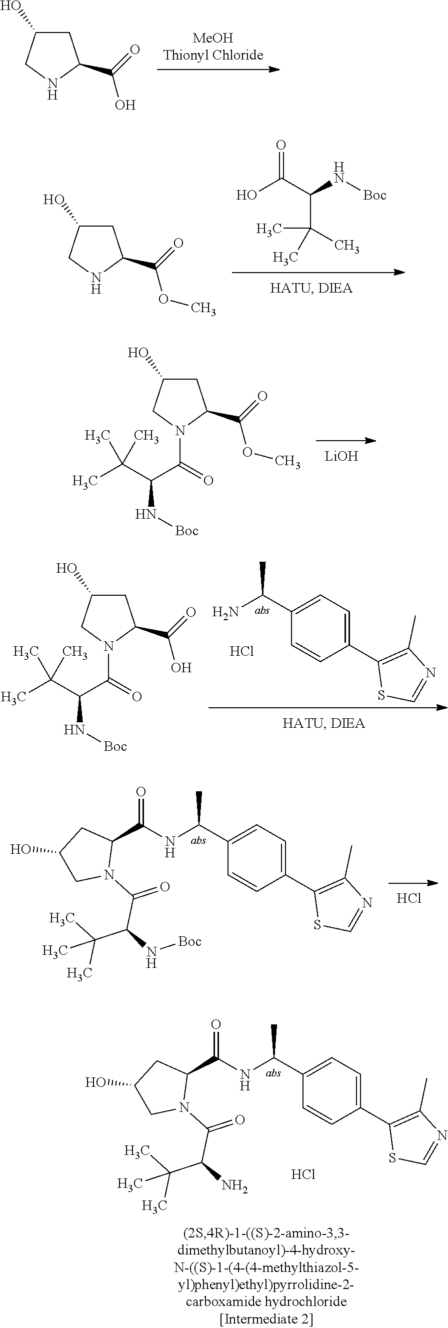

### Step 1: methyl (2S,4R)-4-hydroxypyrrolidine-2-carboxylate

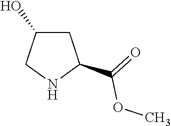

To a solution of (2S,4R)-4-hydroxypyrrolidine-2-carboxylic acid (10.0 g, 76.3 mmol) in MeOH (300 mL) at 0° C., was added SOCl₂ (10.0 mL, 137 mmol) under nitrogen. The mixture was warmed to rt and stirred for 18 h. The volatiles were evaporated under reduced pressure to afford the title compound, which was used in the next step without further purification.

### Step 2: methyl (2S,4R)-1-((S)-2-((tert-butoxycarbonyl)amino)-3,3-dimethylbutanoyl)-4-hydroxypyrrolidine-2-carboxylate

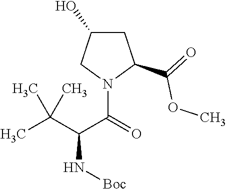 [SMILES: COC(=O)[C@@H]1C[C@@H](O)CN1C(=O)[C@@H](NC(=O)OC(C)(C)C)C(C)(C)C]

To a solution of methyl (2S,4R)-4-hydroxypyrrolidine-2-carboxylate in DCM (250 mL) at rt, were sequentially added (2R)-2-[(tert-butoxycarbonylamino)methyl]-3,3-dimethyl-butanoic acid (18.7 g, 80.9 mmol) and HATU (43.5 g, 114 mmol). The mixture was cooled to 0° C. and then DIEA (65 mL, 380 mmol) was slowly added over 15 min. The reaction mixture was warmed to rt and stirred for 20 h. The mixture was diluted with 5% citric acid (400 mL) and DCM (200 mL) and the layers were separated. The aqueous layer was extracted with DCM (300 mL). The combined organic layers were washed with 1M NaOH (2×200 mL) and brine (200 mL), then dried (Na₂SO₄), filtered and concentrated under reduce pressure to afford the title compound, which was used in the next step without further purification. MS (ESI) [M-BOC]⁺259.3.

### Step 3: (2S,4R)-1-((S)-2-((tert-butoxycarbonyl)amino)-3,3-dimethylbutanoyl)-4-hydroxypyrrolidine-2-carboxylic acid

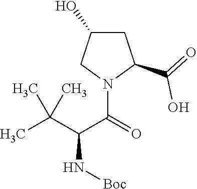 [SMILES: CC(C)(C)OC(=O)N[C@H](C(=O)N1C[C@H](O)C[C@H]1C(=O)O)C(C)(C)C]

To a solution of methyl (2S,4R)-1-[(2S)-2-(tert-butoxycarbonylamino)-3,3-dimethyl-butanoyl]-4-hydroxy-pyrrolidine-2-carboxylate (27.4 g, 76.4 mmol) in MeOH (372 mL) and THF (372 mL) at rt, was added lithium hydroxide monohydrate (7.40 g, 176 mmol) and the mixture was stirred at rt for 48 h. The volatiles were evaporated under reduced pressure. The residue was diluted with 1M NaOH (300 mL) and washed with ether (250 mL). The aqueous layer was acidified to pH 4 and extracted with EtOAc (2×300 mL). The pH was then adjusted to 1 and the mixture was extracted with EtOAc (3×300 mL). The combined organic layers were washed with brine (300 mL), then dried (Na₂SO₄), filtered and reduced under reduced pressure to afford title compound as a foam (31 g), which was used in the next step without further purification. MS (ESI) [M−tBu]⁺289.1.

### Step 4: Tert-butyl N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamate

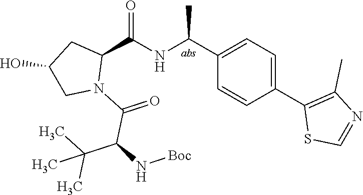 [SMILES: Cc1ncsc1-c1ccc([C@H](C)NC(=O)[C@@H]2C[C@@H](O)CN2C(=O)[C@@H](NC(=O)OC(C)(C)C)C(C)(C)C)cc1]

To a mixture of (2S,4R)-1-[(2S)-2-(tert-butoxycarbonylamino)-3,3-dimethyl-butanoyl]-4-hydroxy-pyrrolidine-2-carboxylic acid (27.3 g, 79.2 mmol), (1S)-1-[4-(4-methylthiazol-5-yl)phenyl]ethanamine hydrochloride (20.2 g, 79.2 mmol) and HATU (45.2 g, 119 mmol) in DCM (775 mL) at 0° C., was slowly added DIEA (68.0 mL, 396 mmol) and the mixture was stirred for 20 h. The mixture was then diluted with 5% citric acid (500 mL) and the layers were separated. The organic layer was washed with 1M NaOH (2×300 mL) and brine (300 mL) then dried (Na₂SO₄), filtered and concentrated under reduced pressure. The resulting solid was dissolved into minimal amount of MeOH and then water was added until precipitation is observed. The resulting solid were filtered, washed with ether (400 mL) and then dried in a vacuum oven at 60° C. to afford the title compound as a solid (34 g, 79%). MS (ESI) [M+H]⁺ 545.3.

### Step 5: (2S,4R)-1-[(2S)-2-amino-3,3-dimethyl-butanoyl]-4-hydroxy-N-[(1S)-1-[4-(4-methylthiazol-5-yl)phenyl]ethyl]pyrrolidine-2-carboxamide hydrochloride

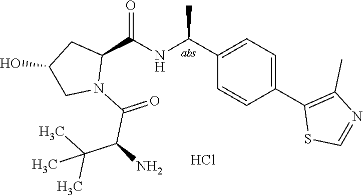 [SMILES: Cc1ncsc1-c1ccc([C@H](C)NC(=O)[C@@H]2C[C@@H](O)CN2C(=O)[C@@H](N)C(C)(C)C)cc1 ; Cl]

To a solution of tert-butyl N-[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]carbamate (34.0 g, 62.0 mmol) in DCM (200 mL) at 0° C., was added an HCl solution (4M in dioxane, 200 mL, 800 mmol) and the mixture was warmed to rt and stirred for 15 min. The mixture was diluted with MeOH (150 mL) and the mixture was stirred for 30 min. The volatiles were evaporated under reduced pressure and coevaporated with PhMe (2×100 mL) to afford the title compound as a solid (30.6 g, 92%, contains 9% PhMe by weight). ¹H NMR (500 MHz, DMSO) δ 9.04 (s, 1H), 8.59 (d, J=7.8 Hz, 1H), 8.09 (d, J=4.3 Hz, 3H), 7.47-7.43 (m, 2H), 7.42-7.37 (m, 2H), 4.93 (p, J=7.0 Hz, 1H), 4.55 (t, J=8.4 Hz, 1H), 4.33 (br s, 1H), 3.91 (q, J=5.7 Hz, 1H), 3.73 (d, J=10.6 Hz, 1H), 3.50 (dd, J=10.9, 3.9 Hz, 1H), 2.70 (s, 1H), 2.47 (s, 3H), 2.12 (dd, J=12.9, 7.7 Hz, 1H), 1.81-1.72 (m, 1H), 1.39 (d, J=7.0 Hz, 3H), 1.03 (s, 9H). MS (ESI) [M+H]⁺ 445.2.

### Example 67: Synthesis of 3-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropoxyl)propanoic acid

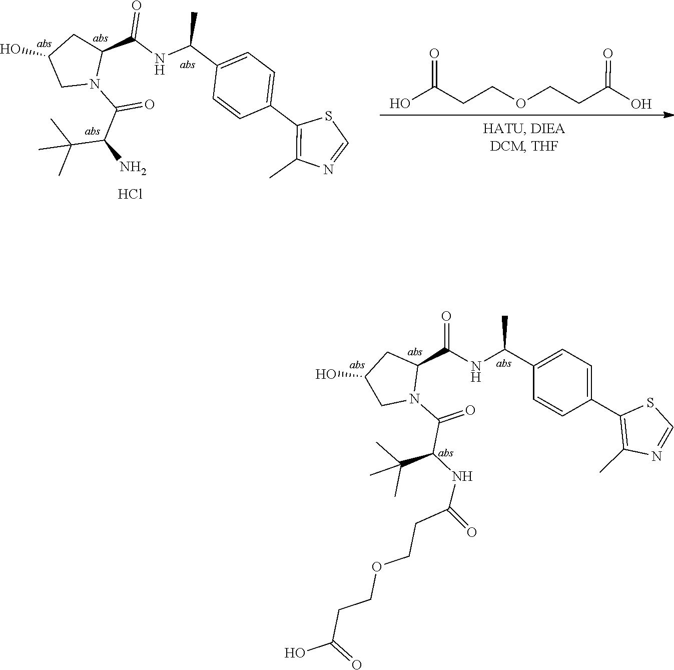 [SMILES: Cc1ncsc1-c1ccc([C@H](C)NC(=O)[C@@H]2C[C@@H](O)CN2C(=O)[C@@H](N)C(C)(C)C)cc1 ; O=C(O)CCOCCC(=O)O ; Cl ; Cc1ncsc1-c1ccc([C@H](C)NC(=O)[C@@H]2C[C@@H](O)CN2C(=O)[C@@H](NC(=O)CCOCCC(=O)O)C(C)(C)C)cc1]

To a solution of 3-(2-carboxyethoxy)propanoic acid (1.5 g, 9.4 mmol) and HATU (2.6 g, 6.9 mmol) in DCM (30 mL) was slowly added DIEA (5.3 mL, 31 mmol) and the solution was stirred for 5 min at rt. To the mixture was added Intermediate 2 (3.0 g, 6.2 mmol) and the reaction mixture was stirred for 30 min. The mixture was diluted with 1M NaOH (5.0 mL) and stirred for 5 min. The mixture was then acidified to pH 5 using 5% citric acid. The layers were separated, and the aqueous layer was extracted with EtOAc (7×50 mL) and DCM (3×50 mL). The combined organic layers were dried (Na₂SO₄), filtered and concentrated under reduced pressure. The material was purified by reverse phase chromatography on C18 using a 10-30% gradient of MeCN and water (contains 0.1% ammonium formate/formic acid) to afford 3-(3-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-3-oxopropoxy)propanoic acid as a solid (1.28 g, 35%). ¹H NMR (500 MHz, DMSO) δ 8.99 (s, 1H), 8.39 (d, J=7.8 Hz, 1H), 7.87 (d, J=9.3 Hz, 1H), 7.47-7.41 (m, 2H), 7.39 (s, 2H), 4.92 (p, J=7.0 Hz, 1H), 4.53 (d, J=9.4 Hz, 1H), 4.44 (t, J=8.0 Hz, 1H), 4.28 (s, 1H), 3.65-3.49 (m, 6H), 2.46 (s, 3H), 2.37 (t, J=6.7 Hz, 2H), 2.39-2.31 (m, 1H), 2.05-1.99 (m, 1H), 1.80 (ddd, J=12.9, 8.4, 4.7 Hz, 1H), 1.37 (t, J=8.2 Hz, 3H), 0.94 (s, 9H). MS (ESI) [M+H]⁺ 589.3.

### Example 68: Synthesis of 3-[2-[2-[2-[2-[3-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-3-oxo-propoxy]ethoxy]ethoxy]ethoxy]ethoxy]propanoic acid

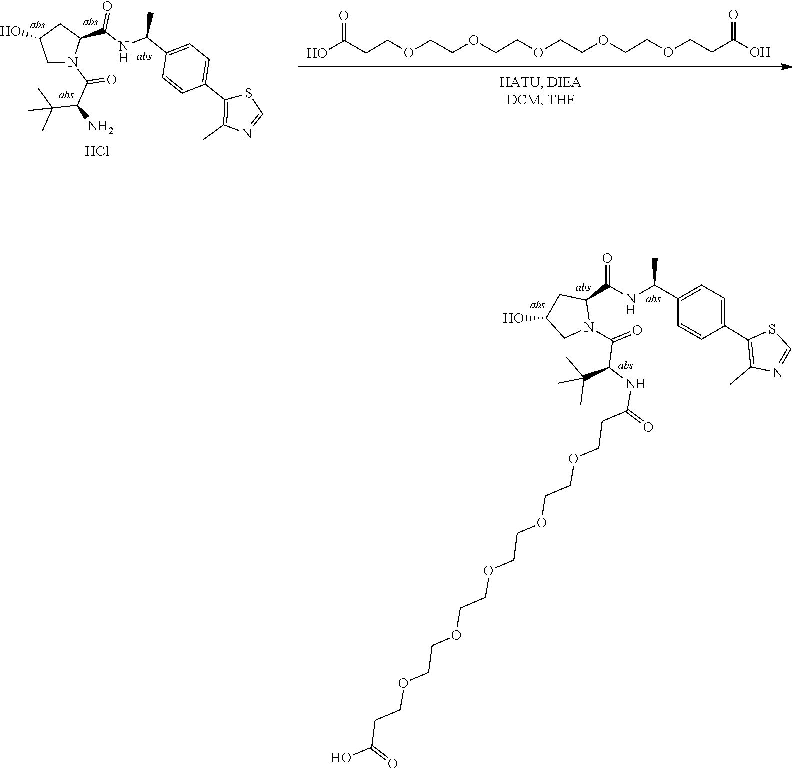 [SMILES: Cc1ncsc1-c1ccc([C@H](C)NC(=O)[C@@H]2C[C@@H](O)CN2C(=O)[C@@H](N)C(C)(C)C)cc1 ; O=C(O)CCOCCOCCOCCOCCOCCC(=O)O ; Cl ; Cc1ncsc1-c1ccc([C@H](C)NC(=O)[C@@H]2C[C@@H](O)CN2C(=O)[C@@H](NC(=O)CCOCCOCCOCCOCCOCCC(=O)O)C(C)(C)C)cc1]

To a solution of 3-[2-[2-[2-[2-(2-carboxyethoxy)ethoxy]ethoxy]ethoxy]ethoxy]propanoic acid (2.0 g, 5.9 mmol) and HATU (1.65 g, 4.34 mmol) in DCM (20 mL) was slowly added DIEA (3.38 mL, 19.7 mmol) and the solution was stirred for 5 min at rt. To the mixture was added Intermediate 2 (1.9 g, 4.0 mmol) and the reaction mixture was stirred for 30 min. The mixture was diluted with 1M NaOH (10 mL) and stirred for 5 min. The mixture was then acidified to pH 5 using 5% citric acid and the layers were separated. The aqueous layer was extracted with EtOAc (7×50 mL) and DCM (3×50 mL). The combined organic layers were dried (Na₂SO₄), filtered and concentrated under reduced pressure. The material was purified by reverse phase chromatography on C18 using a 10-30% gradient of ACN and water (contains 0.1% ammonium formate/formic acid) to afford (S)-21-((2S,4R)-4-hydroxy-2-(((S)-1-(4-(4-methylthiazol-5-yl)phenyl)ethyl)carbamoyl)pyrrolidine-1-carbonyl)-22,22-dimethyl-19-oxo-4,7,10,13,16-pentaoxa-20-azatricosanoic acid as a solid (1.38 g, 46%). ¹H NMR (500 MHz, DMSO) δ 8.99 (s, 1H), 8.43 (s, 1H), 8.39 (d, J=7.4 Hz, 1H), 7.87 (d, J=9.1 Hz, 1H), 7.46-7.42 (m, 2H), 7.41-7.36 (m, 2H), 4.98-4.84 (m, 1H), 4.53 (d, J=9.7 Hz, 1H), 4.43 (t, J=7.7 Hz, 1H), 4.30-4.26 (m, 1H), 3.60 (dd, J=11.9, 5.5 Hz, 6H), 3.54-3.41 (m, 16H), 2.57-2.53 (m, 1H), 2.46 (s, 3H), 2.42 (t, J=6.9 Hz, 2H), 2.36-2.31 (m, 1H), 2.05-1.98 (m, 1H), 1.83-1.76 (m, 1H), 1.38 (d, J=7.1 Hz, 3H), 0.94 (s, 9H). MS (ESI) [M+H]⁺ 765.4.

### Example 69: Synthesis of 7-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-7-oxo-heptanoic acid

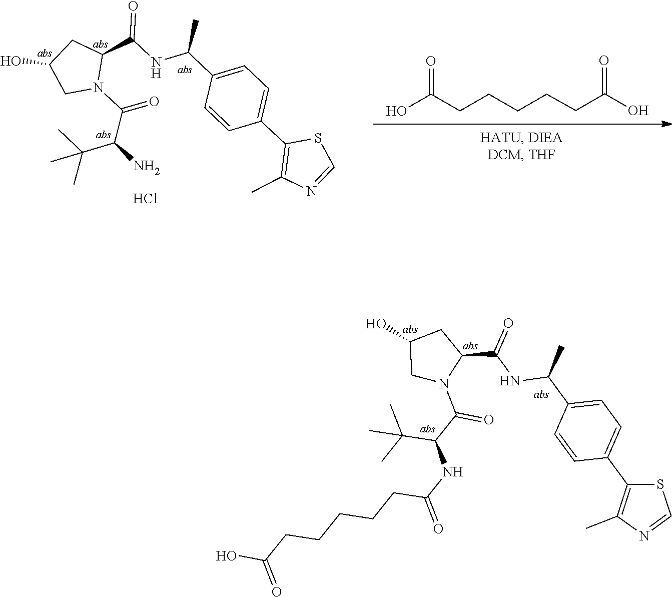 [SMILES: Cc1ncsc1-c1ccc([C@H](C)NC(=O)[C@@H]2C[C@@H](O)CN2C(=O)[C@@H](N)C(C)(C)C)cc1 ; O=C(O)CCCCCC(=O)O ; Cl ; Cc1ncsc1-c1ccc([C@H](C)NC(=O)[C@@H]2C[C@@H](O)CN2C(=O)[C@@H](NC(=O)CCCCCC(=O)O)C(C)(C)C)cc1]

To a solution of Intermediate 2 (1.75 g, 3.64 mmol), heptanedioic acid (874 mg, 5.46 mmol) and HATU (1.94 g, 5.09 mmol) in DCM (70.0 mL) at 0° C., was added DIEA (3.11 mL, 18.2 mmol) and the reaction mixture was stirred for 2 h. The mixture was diluted with 1M NaOH (50 mL) and stirred for 1 h. The layers were separated, and the organic layer was extracted with 1M NaOH (2×30 mL). The combined aqueous layers were acidified to pH 5-6 and extracted with EtOAc (5×50 mL). The combined organic layers were dried (Na₂SO₄), filtered and concentrated under reduced pressure. The material was further purified by reverse phase chromatography on C18 using a 10-60% gradient of MeCN and water (contains 0.1% ammonium formate/formic acid) to afford 7-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-7-oxoheptanoic acid as a solid (0.924 g, 43%). ¹H NMR (500 MHz, DMSO) δ 8.99 (s, 1H), 8.37 (d, J=7.8 Hz, 1H), 7.79 (d, J=9.3 Hz, 1H), 7.46-7.41 (m, 2H), 7.40-7.36 (m, 2H), 4.92 (p, J=7.0 Hz, 1H), 4.52 (d, J=9.4 Hz, 1H), 4.43 (t, J=8.1 Hz, 1H), 4.30-4.26 (m, 1H), 3.65-3.57 (m, 2H), 3.46-3.33 (m, 1H), 2.46 (s, 3H), 2.28-2.20 (m, 1H), 2.18 (t, J=7.4 Hz, 2H), 2.15-2.06 (m, 1H), 2.04-1.97 (m, 1H), 1.80 (ddd, J=12.9, 8.5, 4.7 Hz, 1H), 1.54-1.42 (m, 4H), 1.38 (d, J=7.0 Hz, 3H), 1.28-1.20 (m, 2H), 0.94 (s, 9H). MS (ESI) [M+H]⁺ 587.3.

### Example 70: 9-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-9-oxo-nonanoic acid

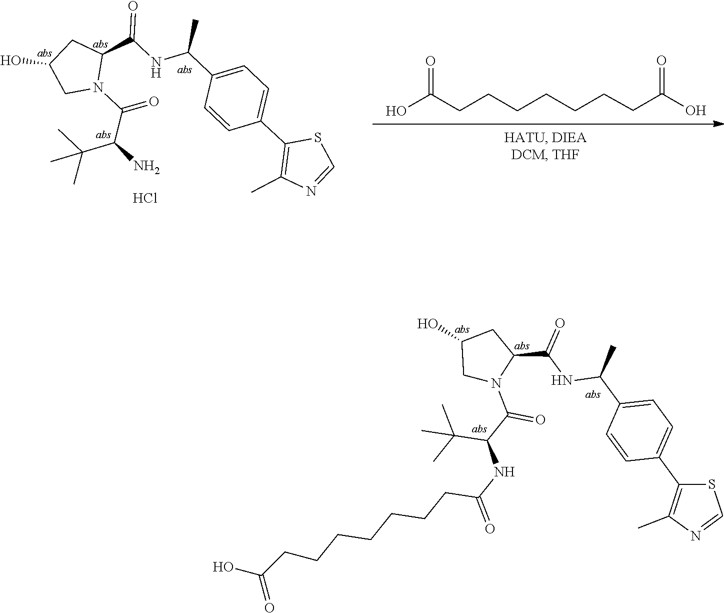 [SMILES: Cc1ncsc1-c1ccc([C@H](C)NC(=O)[C@@H]2C[C@@H](O)CN2C(=O)[C@@H](N)C(C)(C)C)cc1 ; O=C(O)CCCCCCCC(=O)O ; Cl ; Cc1ncsc1-c1ccc([C@H](C)NC(=O)[C@@H]2C[C@@H](O)CN2C(=O)[C@@H](NC(=O)CCCCCCCC(=O)O)C(C)(C)C)cc1]

To a solution of Intermediate 2 (2.0 g, 4.2 mmol), nonanedioic acid (1.2 g, 6.2 mmol) and HATU (2.1 g, 5.4 mmol) in DCM (20 mL) and THF (20 mL) at 0° C., was added DIEA (3.56 mL, 20.8 mmol) and the reaction mixture was stirred for 2 h. The mixture was diluted with 1M NaOH (50 mL) and stirred for 1 h. The mixture was acidified to pH 5 and the aqueous layer was extracted with EtOAc (5×50 mL). The combined organic layers were dried (Na₂SO₄), filtered and concentrated under reduced pressure. The material was purified by reverse phase chromatography on C18 using a10-40% gradient of MeCN and water (contains 0.1% ammonium formate/formic acid) to afford 9-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-9-oxononanoic acid as a solid (1.00 g, 39%). ¹H NMR (500 MHz, DMSO) δ 8.99 (s, 1H), 8.37 (d, J=7.8 Hz, 1H), 7.78 (d, J=9.3 Hz, 1H), 7.47-7.42 (m, 2H), 7.40-7.36 (m, 2H), 5.10 (br s, 1H), 4.97-4.88 (m, 1H), 4.52 (d, J=9.3 Hz, 1H), 4.43 (t, J=8.0 Hz, 1H), 4.33-4.24 (m, 1H), 3.66-3.54 (m, 2H), 2.46 (s, 3H), 2.28-2.22 (m, 1H), 2.19 (t, J=7.4 Hz, 2H), 2.14-2.07 (m, 1H), 2.04-1.98 (m, 1H), 1.83-1.76 (m, 1H), 1.54-1.41 (m, 4H), 1.38 (d, J=7.0 Hz, 3H), 1.31-1.19 (m, 6H), 0.94 (s, 9H). MS (ESI) [M+H]⁺ 615.7.

### Example 71: 11-[[(1S)-1-[(2S,4R)-4-hydroxy-2-[[(1S)-1-[4-(4-methylthiazol-5-yl)phenyl]ethyl]carbamoyl]pyrrolidine-1-carbonyl]-2,2-dimethyl-propyl]amino]-11-oxo-undecanoic acid

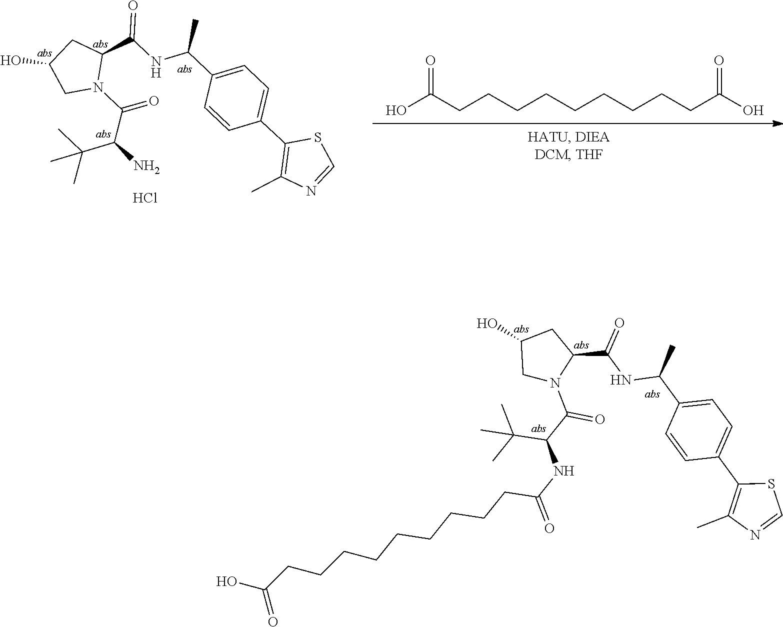 [SMILES: Cc1ncsc1-c1ccc([C@H](C)NC(=O)[C@@H]2C[C@@H](O)CN2C(=O)[C@@H](N)C(C)(C)C)cc1 ; O=C(O)CCCCCCCCCC(=O)O ; Cl ; Cc1ncsc1-c1ccc([C@H](C)NC(=O)[C@@H]2C[C@@H](O)CN2C(=O)[C@@H](NC(=O)CCCCCCCCCC(=O)O)C(C)(C)C)cc1]

To a solution of Intermediate 2 (2.0 g, 4.2 mmol), undecanedioic acid (1.4 g, 6.2 mmol) and HATU (2.4 g, 6.2 mmol) in DCM (20 mL) and THE (20 mL) at 0° C., was added DIEA (3.56 mL, 20.8 mmol) and the reaction mixture was stirred for 2 h. The mixture was diluted with 1M NaOH (50 mL) and stirred for 1 h. The mixture was acidified to pH 5 and the aqueous layer was extracted with EtOAc (5×50 mL). The combined organic layers were dried (Na₂SO₄), filtered and concentrated under reduced pressure. The material was purified by reverse phase chromatography on C18 using a10-40% gradient of MeCN and water (contains 0.1% ammonium formate/formic acid) to afford 11-(((S)-1-((2S,4R)-4-hydroxy-2-(((S)-1-(4-(4-methylthiazol-5-yl)phenyl)ethyl)carbamoyl)pyrrolidin-1-yl)-3,3-dimethyl-1-oxobutan-2-yl)amino)-11-oxoundecanoic acid as a solid (832 mg, 31%). ¹H NMR (500 MHz, DMSO) δ 8.99 (s, 1H), 8.37 (d, J=7.8 Hz, 1H), 7.77 (d, J=9.4 Hz, 1H), 7.46-7.42 (m, 2H), 7.41-7.36 (m, 2H), 5.09 (br s, 1H), 4.95-4.88 (m, 1H), 4.52 (d, J=9.4 Hz, 1H), 4.43 (t, J=8.0 Hz, 1H), 4.31-4.25 (m, 1H), 3.67-3.54 (m, 2H), 2.46 (s, 3H), 2.30-2.21 (m, 1H), 2.19 (t, J=7.4 Hz, 2H), 2.14-2.06 (m, 1H), 2.04-1.98 (m, 1H), 1.80 (ddd, J=12.9, 8.4, 4.6 Hz, 1H), 1.54-1.42 (m, 4H), 1.38 (d, J=7.0 Hz, 3H), 1.30-1.18 (m, 10H), 0.94 (s, 9H). MS (ESI) [M+H]⁺ 643.4.

### Example 72: Preparation of 3-(3-(((3S,5S)-1-((S)-2-((S)-2-((tert-butoxycarbonyl)(methyl)amino)propanamido)-2-cyclohexylacetyl)-5-(((R)-1,2,3,4-tetrahydronaphthalen-1-yl)carbamoyl)pyrrolidin-3-yl)amino)-3-oxopropoxy)propanoic acid

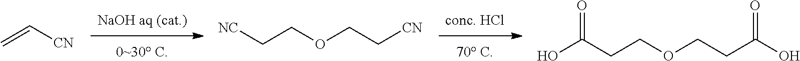

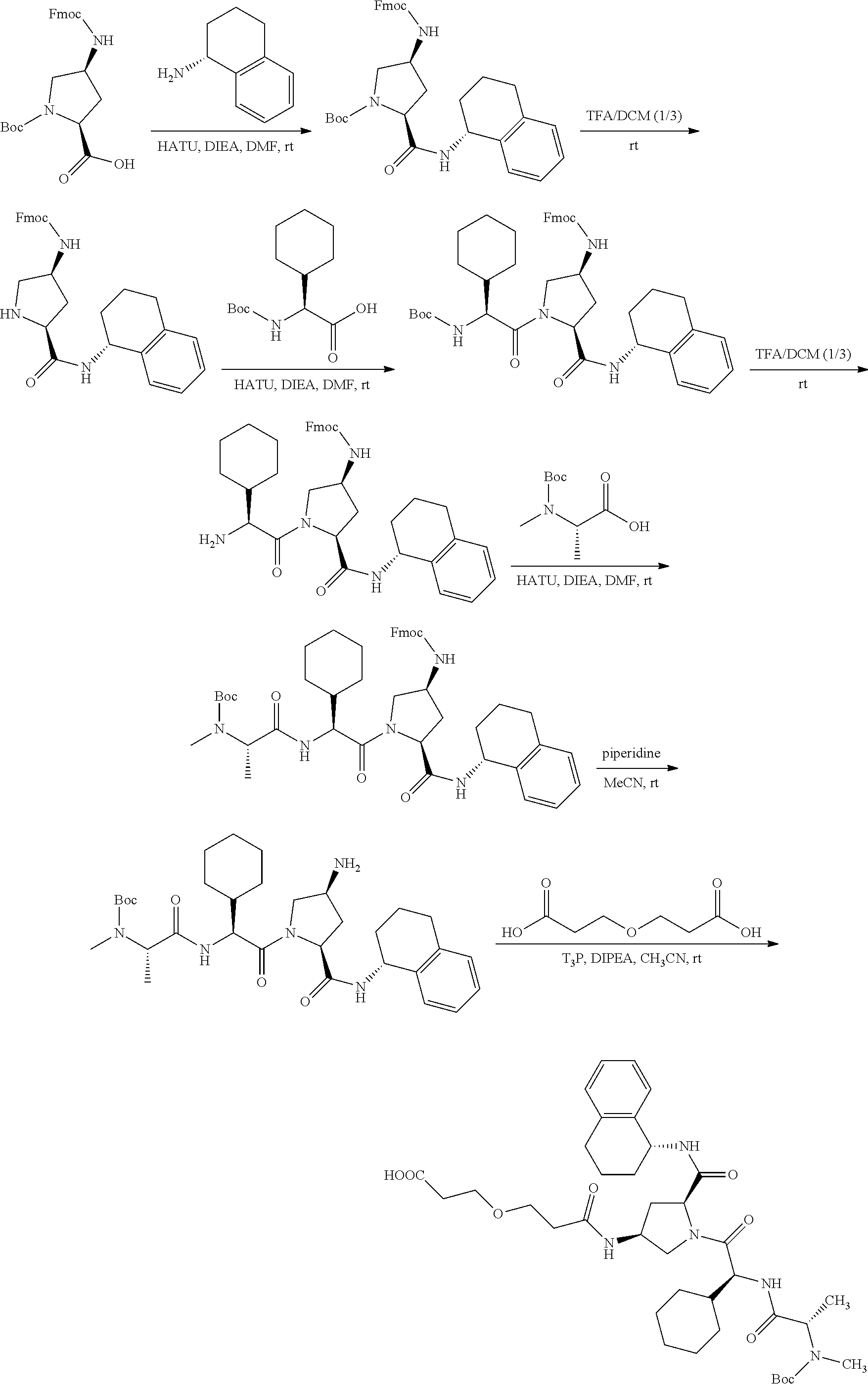

### Step 1: 3,3′-oxydipropanenitrile

To a stirred solution of NaOH aqueous (3 mL, 40% wt) was added acrylonitrile (17.5 g, 330 mmol) dropwise at 0° C. The solution was stirred at 30° C. for 16 h. When the reaction was completed, the reaction was diluted with 100 mL H₂O and neutralized to pH 7 by HCl (2 N). The aqueous solution was extracted with ethyl acetate (100 mL×3). The combined organic solution was dried over anhydrous Na₂SO₄ and concentrated to give 3,3′-oxydipropanenitrile (4.1 g, crude) as yellow oil, which was used for the next step without further purification. ¹H NMR (300 MHz, Chloroform-d) δ 3.74 (t, J=6.3 Hz, 4H), 2.65 (t, J=6.3 Hz, 4H).

### Step 2: 3,3′-oxydipropionic acid

A mixture of 3,3′-oxydipropanenitrile (4.1 g, 33 mmol) and concentrated HCl (38 mL) was stirred at 70° C. for 16 h. After cooled to room temperature, the solids were filtered and the filtrate was concentrated under vacuum. The crude residue was purified by flash column chromatography with 30˜100% ethyl acetate in petroleum ether to afford 3,3′-oxydipropionic acid (3.2 g, 12% over 2 steps) as yellow oil. ¹H NMR (400 MHz, DMSO-d₆) δ 12.20 (s, 2H), 3.62-3.55 (m, 4H), 2.42-2.40 (m, 4H).

### Step 3: (2S,4S)-tert-butyl 4-(((9H-fluoren-9-yl)methoxy)carbonylamino)-2-((R)-1,2,3,4-tetrahydronaphthalen-1-ylcarbamoyl)pyrrolidine-1-carboxylate

To a solution of (2S,4S)-4-(((9H-fluoren-9-yl)methoxy)carbonylamino)-1-(tert-butoxycarbonyl)pyrrolidine-2-carboxylic acid (10 g, 22.2 mmol), (R)-1,2,3,4-tetrahydronaphthalen-1-amine (3.26 g, 22.2 mmol) and DIEA (14.28 g, 111 mmol) in DMF (100 mL) was added HATU (9.26 g, 24.4 mmol). The solution was stirred at room temperature for 3 h. The reaction was quenched by the addition of 200 mL H₂O and then extracted with ethyl acetate (200 mL×3). The combined organic layer was washed with brine, dried over anhydrous Na₂SO₄ and concentrated under vacuum. The crude residue was purified by flash column chromatography with 10˜50% ethyl acetate in petroleum ether to afford (2S,4S)-tert-butyl 4-(((9H-fluoren-9-yl)methoxy)carbonylamino)-2-((R)-1,2,3,4-tetrahydronaphthalen-1-ylcarbamoyl)pyrrolidine-1-carboxylate (12.0 g, 93%) as a white solid. MS (ESI) calculated for (C₃₅H₃₉N₃O₅) [M+H]⁺, 582.3; found, 582.0.

### Step 4: (9H-fluoren-9-yl)methyl ((3S,5S)-5-(((R)-1,2,3,4-tetrahydronaphthalen-1-yl)carbamoyl)pyrrolidin-3-yl)carbamate TFA salt

To a stirred solution of (2S,4S)-tert-butyl 4-(((9H-fluoren-9-yl)methoxy)carbonylamino)-2-((R)-1,2,3,4-tetrahydronaphthalen-1-ylcarbamoyl)pyrrolidine-1-carboxylate (12 g, 26.54 mmol) in DCM (120 mL) was added TFA (40 mL) at room temperature. The resulting mixture was stirred at room temperature overnight. The solvent was removed under vacuum to afford (9H-fluoren-9-yl)methyl ((3S,5S)-5-(((R)-1,2,3,4-tetrahydronaphthalen-1-yl)carbamoyl)pyrrolidin-3-yl)carbamate TFA salt (13 g, crude) as yellow oil, which was used for the next step without further purification. MS (ESI) calculated for (C₃₀H₃₁N₃O₃) [M+H]⁺, 482.2; found, 482.0.

### Step 5: 9H-fluoren-9-ylmethyl N-[(3S,5S)-1-[(2S)-2-[[(tert-butoxy)carbonyl]amino]-2-cyclohexylacetyl]-5-[[(1R)-1,2,3,4-tetrahydronaphthalen-1-yl]carbamoyl]pyrrolidin-3-yl]carbamate

To a stirred solution of (9H-fluoren-9-yl)methyl (3S,5S)-5-((R)-1,2,3,4-tetrahydronaphthalen-1-ylcarbamoyl)pyrrolidin-3-ylcarbamate TFA salt (13 g, 27.0 mmol), DIEA (17.44 g, 135 mmol) and (S)-2-(tert-butoxycarbonylamino)-2-cyclohexylacetic acid (6.95 g, 27.0 mmol) in DMF (150 mL) was added HATU (12.33 g, 32.4 mmol). The resulting mixture was stirred at room temperature for 4 h. The reaction was quenched by the addition of 200 mL H₂O and extracted with ethyl acetate (200 mL×3). The combined organic layer was washed with brine, dried over anhydrous Na₂SO₄ and concentrated under vacuum. The crude residue was purified by flash column chromatography with 10-40% ethyl acetate in petroleum ether to afford 9H-fluoren-9-ylmethyl N-[(3S,5S)-1-[(2S)-2-[[(tert-butoxy)carbonyl]amino]-2-cyclohexylacetyl]-5-[[(1R)-1,2,3,4-tetrahydronaphthalen-1-yl]carbamoyl]pyrrolidin-3-yl]carbamate (5.2 g, 27%) as colorless oil. MS (ESI) calculated for (C₄₃H₅₂N₄O₆) [M+H]⁺, 721.4; found, 721.0.

### Step 6: (9H-fluoren-9-yl)methyl (3S,5S)-1-((S)-2-amino-2-cyclohexylacetyl)-5-((R)-1,2,3,4-tetrahydronaphthalen-1-ylcarbamoyl)pyrrolidin-3-ylcarbamate TFA salt

To a stirred solution of 9H-fluoren-9-ylmethyl N-[(3S,5S)-1-[(2S)-2-[[(tert-butoxy)carbonyl]amino]-2-cyclohexylacetyl]-5-[[(1R)-1,2,3,4-tetrahydronaphthalen-1-yl]carbamoyl]pyrrolidin-3-yl]carbamate (5.2 g, 7.22 mmol) in DCM (90 mL) was added TFA (30 mL). The solution was stirred at room temperature overnight. The solvents were removed under vacuum to afford (9H-fluoren-9-yl)methyl (3S,5S)-1-((S)-2-amino-2-cyclohexylacetyl)-5-((R)-1,2,3,4-tetrahydronaphthalen-1-ylcarbamoyl)pyrrolidin-3-ylcarbamate TFA salt (4.48 g, crude) as yellow oil. MS (ESI) calculated for (C₃₈H₄₄N₄O₄) [M+H]⁺, 621.3; found, 621.0.

### Step 7: 9H-fluoren-9-ylmethyl N-[(3S,5S)-1-[(2S)-2-[(2S)-2-[[(tert-butoxy)carbonyl](methyl)amino]propanamido]-2-cyclohexylacetyl]-5-[[(1R)-1,2,3,4-tetrahydronaphthalen-1-yl]carbamoyl]pyrrolidin-3-yl]carbamate

To a stirred solution of (9H-fluoren-9-yl)methyl (3S,5S)-1-((S)-2-amino-2-cyclohexylacetyl)-5-((R)-1,2,3,4-tetrahydronaphthalen-1-ylcarbamoyl)pyrrolidin-3-ylcarbamate (4.48 g, 7.22 mmol), DIEA (4.66 g, 36.1 mmol) and (S)-2-(tert-butoxycarbonyl(methyl)amino)propanoic acid (1.46 g, 7.22 mmol) in DMF (50 mL) was added HATU (3.3 g, 8.68 mmol). The resulting mixture was stirred at room temperature for 3 h. The reaction was quenched by the addition of 100 mL H₂O and extracted with ethyl acetate (100 mL×3). The combined organic layer was washed with brine, dried over anhydrous Na₂SO₄ and concentrated under vacuum. The crude residue was purified by flash column chromatography with 20-60% ethyl acetate in petroleum ether to afford 9H-fluoren-9-ylmethyl N-[(3S,5S)-1-[(2S)-2-[(2S)-2-[[(tert-butoxy)carbonyl](methyl)amino]propanamido]-2-cyclohexylacetyl]-5-[[(1R)-1,2,3,4-tetrahydronaphthalen-1-yl]carbamoyl]pyrrolidin-3-yl]carbamate (5.2 g, 89%) as colorless oil. MS (ESI) calculated for (C₄₇H₅₉N₅O₇) [M+H]⁺, 806.4; found, 806.0.

### Step 8: tert-butyl (S)-1-((S)-2-((2S,4S)-4-amino-2-((R)-1,2,3,4-tetrahydronaphthalen-1-ylcarbamoyl)pyrrolidin-1-yl)-1-cyclohexyl-2-oxoethylamino)-1-oxopropan-2-yl(methyl)carbamate

To a stirred solution of 9H-fluoren-9-ylmethyl N-[(3S,5S)-1-[(2S)-2-[(2S)-2-[[(tert-butoxy)carbonyl](methyl)amino]propanamido]-2-cyclohexylacetyl]-5-[[(1R)-1,2,3,4-tetrahydronaphthalen-1-yl]carbamoyl]pyrrolidin-3-yl]carbamate (5.2 g, 6.46 mmol) in acetonitrile (80 mL) was added piperidine (5.2 mL). The mixture was stirred at room temperature for 1 h. The solids were filtered out by filtration and the filtrate was concentrated under vacuum. The crude residue was purified by reverse phase flash column chromatography with 5˜95% acetonitrile in water to afford tert-butyl (S)-1-((S)-2-((2S,4S)-4-amino-2-((R)-1,2,3,4-tetrahydronaphthalen-1-ylcarbamoyl)pyrrolidin-1-yl)-1-cyclohexyl-2-oxoethylamino)-1-oxopropan-2-yl(methyl)carbamate (3.1656 g, 84%) as a white solid. ¹H NMR (300 MHz, DMSO-d6) δ 8.45-8.12 (m, 1H), 7.71 (m, 1H), 7.39-6.99 (m, 4H), 4.94-4.91 (m, 1H), 4.61-4.45 (m, 1H), 4.34-4.19 (m, 2H), 3.90-3.88 (m, 1H), 3.29-3.16 (m, 1H), 2.75-2.72 (m, 5H), 2.50-2.27 (m, 1H), 2.01-1.82 (m, 4H), 1.81-1.50 (m, 9H), 1.41 (s, 9H), 1.29-0.85 (m, 9H). MS (ESI) calculated for (C₃₂H₄₉N₅O₅) [M+H]⁺, 584.4; found, 584.4.

### Step 9: 3-(3-(((3S,5S)-1-((S)-2-((S)-2-((tert-butoxycarbonyl)(methyl)amino)propanamido)-2-cyclohexylacetyl)-5-(((R)-1,2,3,4-tetrahydronaphthalen-1-yl)carbamoyl)pyrrolidin-3-yl)amino)-3-oxopropoxy)propanoic acid

To a stirred solution of tert-butyl ((S)-1-(((S)-2-((2S,4S)-4-amino-2-(((R)-1,2,3,4-tetrahydronaphthalen-1-yl)carbamoyl)pyrrolidin-1-yl)-1-cyclohexyl-2-oxoethyl)amino)-1-oxopropan-2-yl)(methyl)carbamate (1.5 g, 2.57 mmol), 3,3′-oxydipropionic acid (2.78 g, 12.86 mmol) and DIEA (1.65 g, 12.86 mmol) in acetonitrile (30 mL) was added T₃P (12.3 g, 10.28 mmol, 50% in ethyl acetate) under nitrogen. The solution was stirred at 20° C. for 16 h. When the reaction was completed, the reaction was quenched by the addition of 50 mL H₂O and the aqueous solution was extracted with ethyl acetate (50 mL×3). The combined organic layer was washed with brine, dried over anhydrous Na₂SO₄ and concentrated under vacuum. The crude residue was purified by reverse phase flash column chromatography with 5˜50% acetonitrile in water to afford 3-(3-(((3S,5S)-1-((S)-2-((S)-2-((tert-butoxycarbonyl)(methyl)amino)propanamido)-2-cyclohexylacetyl)-5-(((R)-1,2,3,4-tetrahydronaphthalen-1-yl)carbamoyl)pyrrolidin-3-yl)amino)-3-oxopropoxy)propanoic acid (1.0929 g, 58%) as a white solid. ¹H NMR (400 MHz, Methanol-d₄) δ 7.48-7.36 (m, 1H), 7.23-7.03 (m, 3H), 5.07-5.06 (m, 1H), 4.63-4.30 (m, 4H), 4.21-4.18 (m, 1H), 3.72-3.67 (m, 4H), 3.55-3.51 (m, 1H), 2.91 (s, 3H), 2.91-2.73 (m, 2H), 2.67-2.41 (m, 5H), 2.04-1.61 (m, 11H), 1.49 (s, 9H), 1.38-1.00 (m, 8H). MS (ESI) calculated for (C₃₈H₅₇N₅O₉) [M+H]⁺, 728.4; found, 728.7.

### Example 73: 3-(2-(2-(3-(((3S,5S)-1-((S)-2-((S)-2-((tert-butoxycarbonyl)(methyl)amino)propanamido)-2-cyclohexylacetyl)-5-(((R)-1,2,3,4-tetrahydronaphthalen-1-yl)carbamoyl)pyrrolidin-3-yl)amino)-3-oxopropoxy)ethoxy)ethoxy)propanoic acid

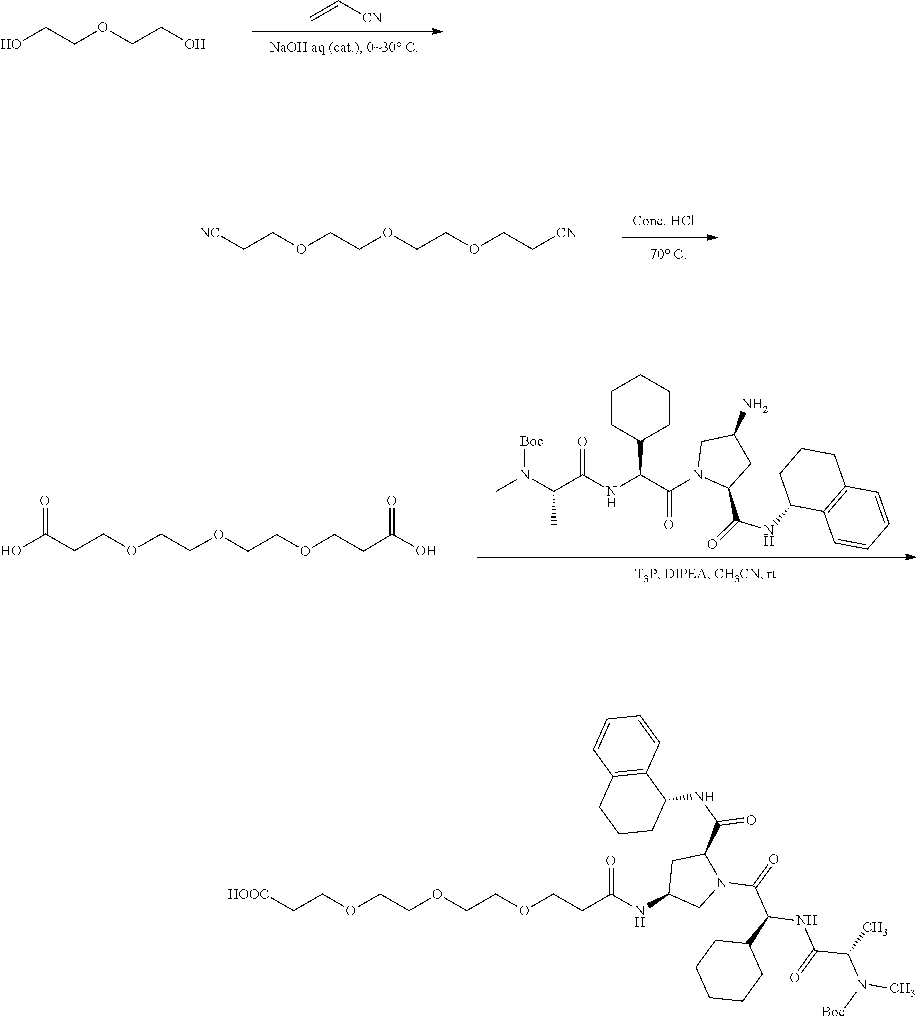

### Step 1: 3,3′-((oxybis(ethane-2,1-diyl))bis(oxy))dipropanenitrile

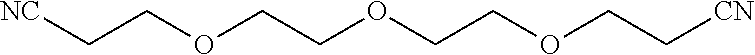 [SMILES: N#CCCOCCOCCOCCC#N]

To a stirred solution of 2,2′-oxybis(ethan-1-ol) (15 g, 141 mmol) and NaOH aqueous (1.7 mL, 40% wt) was added acrylonitrile (17.25 g, 325 mmol) dropwise at 0° C. The solution was stirred at 30° C. for 16 h. When the reaction was completed, the reaction was diluted with 100 mL H₂O and neutralized to pH 7 by HCl (2 N). The aqueous solution was extracted with ethyl acetate. The combined organic layer was dried over anhydrous Na₂SO₄ and concentrated under vacuum to afford 3,3′-((oxybis(ethane-2,1-diyl))bis(oxy))dipropanenitrile (26 g, crude) as yellow oil, which was used for the next step without further purification. ¹H NMR (300 MHz, Chloroform-d) δ 3.72 (t, J=6.3 Hz, 4H), 3.67 (s, 8H), 2.62 (t, J=6.3 Hz, 4H).

### Step 2: 3,3′-((oxybis(ethane-2,1-diyl))bis(oxy))dipropionic acid

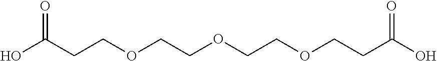 [SMILES: O=C(O)CCOCCOCCOCCC(=O)O]

A mixture of 3,3′-((oxybis(ethane-2,1-diyl))bis(oxy))dipropanenitrile (26 g, 123 mmol) and concentrated HCl (140 mL) was stirred at 70° C. overnight. After cooled to room temperature, the solids were filtered out by filtration and the filtrate was concentrated under vacuum. The crude residue was purified by flash column chromatography with 30˜100% ethyl acetate in petroleum ether to afford 3,3′-((oxybis(ethane-2,1-diyl))bis(oxy))dipropionic acid (20.9 g, 70% over 2 steps) as yellow oil. ¹H NMR (400 MHz, DMSO-d6) δ 12.16 (s, 2H), 3.61-3.57 (m, 4H), 3.51-3.47 (m, 8H), 2.44 (t, J=6.3 Hz, 4H).

### Step 3: 3-(2-(2-(3-(((3S,5S)-1-((S)-2-((S)-2-((tert-butoxycarbonyl)(methyl)amino)propanamido)-2-cyclohexylacetyl)-5-(((R)-1,2,3,4-tetrahydronaphthalen-1-yl)carbamoyl)pyrrolidin-3-yl)amino)-3-oxopropoxy)ethoxy)ethoxy)propanoic acid

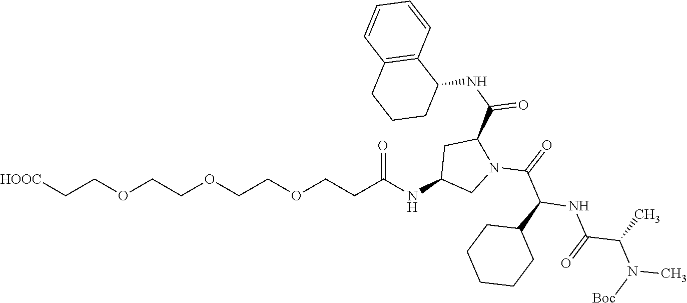 [SMILES: C[C@@H](C(=O)N[C@H](C(=O)N1C[C@@H](NC(=O)CCOCCOCCOCCC(=O)O)C[C@H]1C(=O)N[C@@H]1CCCc2ccccc21)C1CCCCC1)N(C)C(=O)OC(C)(C)C]

To a stirred solution of tert-butyl ((S)-1-(((S)-2-((2S,4S)-4-amino-2-(((R)-1,2,3,4-tetrahydronaphthalen-1-yl)carbamoyl)pyrrolidin-1-yl)-1-cyclohexyl-2-oxoethyl)amino)-1-oxopropan-2-yl)(methyl)carbamate (1.0 g, 1.71 mmol), 3,3′-((oxybis(ethane-2,1-diyl))bis(oxy))dipropionic acid (1.15 g, 3.43 mmol) and DIEA (1.1 g, 8.57 mmol) in acetonitrile (20 mL), was added T₃P (8.66 g, 6.86 mmol, 50% in EtOAc) under nitrogen. The resulting mixture was stirred at room temperature for 16 h. When the reaction was completed, the reaction was quenched by the addition of 50 mL H₂O. The aqueous solution was extracted with ethyl acetate. The combined organic layer was washed with brine, dried over anhydrous Na₂SO₄ and concentrated under vacuum. The residue was purified by pre-HPLC with the following conditions: [(Column: X Bridge Prep OBD C18 Column 30×150 mm Sum; Mobile Phase A: Water (10 mmol/L NH₄HCO₃), Mobile Phase B: ACN; Flow rate: 60 mL/min; Gradient: 28% B to 44% B in 7 min; 254/220 nm] to afford 3-(2-(2-(3-(((3S,5S)-1-((S)-2-((S)-2-((tert-butoxycarbonyl)(methyl)amino)propanamido)-2-cyclohexylacetyl)-5-(((R)-1,2,3,4-tetrahydronaphthalen-1-yl)carbamoyl)pyrrolidin-3-yl)amino)-3-oxopropoxy)ethoxy)ethoxy)propanoic acid (215.4 mg, 15%) as a white solid. ¹H NMR (300 MHz, DMSO-d₆) δ 8.39 (d, J=8.7 Hz, 1H), 8.22 (d, J=7.5 Hz, 1H), 7.80-7.70 (m, 1H), 7.32 (d, J=7.2 Hz, 1H), 7.22-6.98 (m, 3H), 4.94-4.92 (m, 1H), 4.51-4.49 (m, 1H), 4.28-4.26 (m, 3H), 4.09 (t, J=8.7 Hz, 1H), 3.60-3.58 (m, 4H), 3.49 (s, 8H), 2.75-2.73 (m, 5H), 2.35-2.31 (m, 5H), 1.99-1.50 (m, 11H), 1.40 (s, 9H), 1.30-0.82 (m, 9H). MS (ESI) calculated for (C₄₂H₆₅N₅O₁₁) [M+H]⁺, 816.5; found, 816.5.

### Example 74: (S)-2-((S)-2-((tert-butoxycarbonyl)(methyl)amino) propanamido)-2-cyclohexylacetic acid

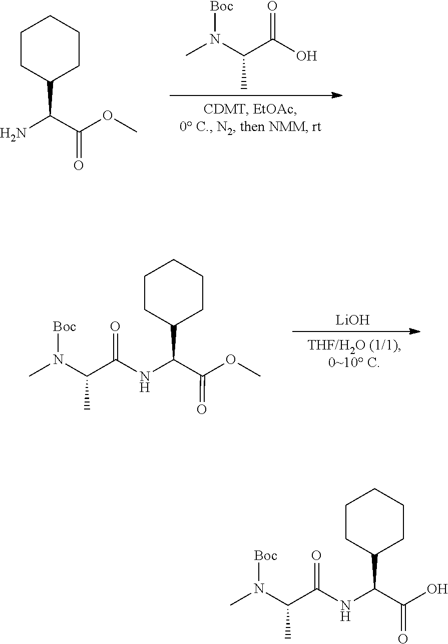

### Step 1: methyl (S)-2-((S)-2-((tert-butoxycarbonyl)(methyl)amino)propanamido)-2-cyclohexylacetate

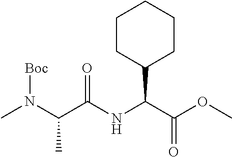

A solution of (S)-methyl-2-amino cyclohexyl acetate hydrochloride (70.0 g, 0.34 mol) and (S)-2-(tert-butoxycarbonyl(methyl)amino)propanoic acid (69.0 g, 0.34 mol) in ethyl acetate (300 mL) was treated with 2-chloro-4,6-dimethoxy-1,3,5-triazine (CDMT) (64.7 g, 0.37 mol) under nitrogen. The reaction mixture was cooled to 0° C. and treated with N-methylmorpholine (85.8 g, 0.85 mol). The reaction mixture was warmed to room temperature and stirred for 4 h. The solid precipitate was filtered out and rinsed with ethyl acetate. The filtrate was washed with saturated NaHCO₃ aqueous solution and then 10% citric acid and brine, dried over anhydrous sodium sulfate and concentrated under vacuum to afford methyl (S)-2-((S)-2-((tert-butoxycarbonyl)(methyl)amino)propanamido)-2-cyclohexylacetate (85.0 g, 71%) as an off-white solid. MS (ESI) calculated for (C₁₈H₃₂N₂O₅) [M+H]⁺, 357.2; found, 357.0.

### Step 2: (S)-2-((S)-2-((tert-butoxycarbonyl)(methyl)amino)propanamido)-2-cyclohexylacetic acid

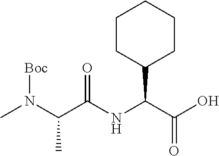 [SMILES: C[C@@H](C(=O)N[C@H](C(=O)O)C1CCCCC1)N(C)C(=O)OC(C)(C)C]

To a solution of methyl (S)-2-((S)-2-((tert-butoxycarbonyl)(methyl)amino) propanamido)-2-cyclohexylacetate (85.0 g, 0.24 mol) in THE (1.2 L) was added a solution of LiOH—H₂O (25.2 g, 0.60 mol) in water (1.2 L) maintained the temperature at 0-10° C. under nitrogen. The resulting mixture was stirred at 0-10° C. for 3 h. The organic solvent was removed under vacuum and the pH value of aqueous phase was adjusted to ˜3 by citric acid. The mixture was extracted with ethyl acetate twice. The combined organic layer was washed with brine, dried over anhydrous sodium sulfate and concentrated under vacuum to afford (S)-2-((S)-2-((tert-butoxycarbonyl)(methyl)amino)propanamido)-2-cyclohexylacetic acid (100 g, crude) as colorless oil, which was used for the next step without further purification. MS (ESI) calculated for (C₁₇H₃₀N₂O₅) [M−H], 341.2; found, 341.0.

### Example 75: tert-butyl ((S)-1-(((S)-1-cyclohexyl-2-((S)-2-(4-(3-hydroxybenzoyl)thiazol-2-yl)pyrrolidin-1-yl)-2-oxoethyl)amino)-1-oxopropan-2-yl)(methyl)carbamate

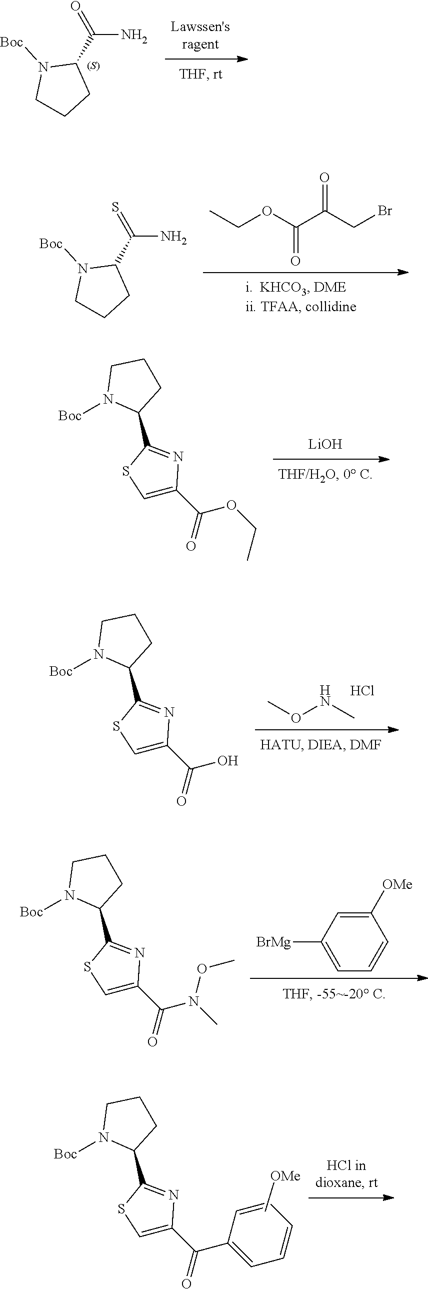

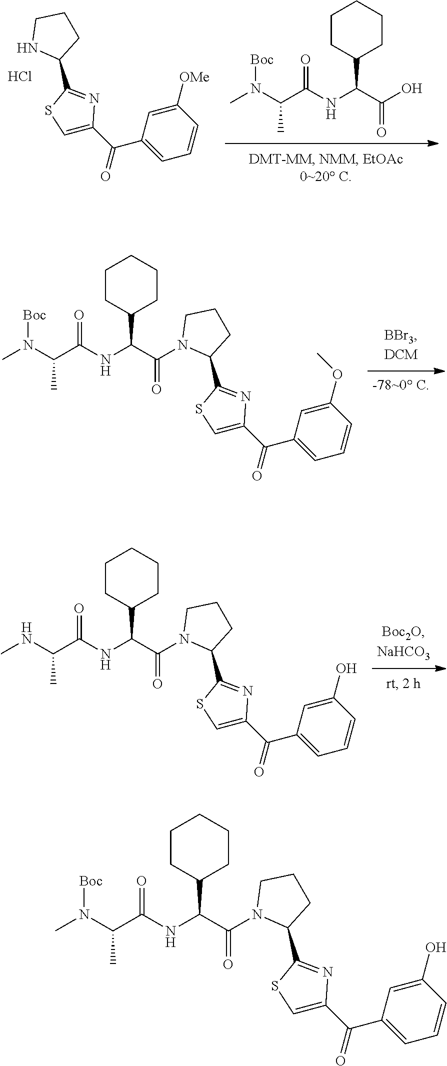

### Step 1: tert-butyl (S)-2-carbamothioylpyrrolidine-1-carboxylate

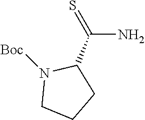

To a solution of tert-butyl (2S)-2-carbamoylpyrrolidine-1-carboxylate (100 g, 466.72 mmol) in tetrahydrofuran (1.2 L) was added lawesson's reagent (113 g, 279.70 mmol). The resulting mixture was stirred at room temperature for 16 h. The mixture was then diluted with saturated NaHCO₃ aqueous solution and extracted with ethyl acetate. The combined organic layer was washed with brine, dried over anhydrous sodium sulfate and concentrated under vacuum to afford tert-butyl (S)-2-carbamothioylpyrrolidine-1-carboxylate (110 g, crude) as a white solid, which was used for the next step without further purification. MS (ESI) calculated for (C₁₀H₁₈N₂O₂S) [M+H]⁺, 231.1; found, 231.0.

### Step 2: ethyl (S)-2-(1-(tert-butoxycarbonyl)pyrrolidin-2-yl)thiazole-4-carboxylate

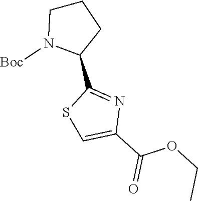 [SMILES: CCOC(=O)c1csc([C@@H]2CCCN2C(=O)OC(C)(C)C)n1]

To a mixture of tert-butyl (S)-2-carbamothioylpyrrolidine-1-carboxylate (100.0 g, 0.44 mol) and potassium bicarbonate (348.0 g, 3.48 mol) in dimethoxyethane (1.5 L) was added ethyl 3-bromo-2-oxopropanoate (253.1 g, 1.30 mol) dropwise at room temperature. The resulting mixture was stirred at room temperature for 1 h and then cooled to 0° C. And then trifluoroacetic acid (365.4 g, 1.74 mol) and collidine (298.2 g, 2.78 mol) were added dropwise to the above solution at 0° C. The resulting mixture was stirred at room temperature for 8 h. The reaction was quenched by the addition of water and the aqueous phase was extracted with dichloromethane. The combined organic layer was washed with HCl (0.5 N) and brine, dried over anhydrous sodium sulfate and concentrated under vacuum. The crude residue was purified by flash column chromatography with 10-30% ethyl acetate in petroleum ether to afford ethyl (S)-2-(1-(tert-butoxycarbonyl)pyrrolidin-2-yl)thiazole-4-carboxylate (51.5 g, 34% over two steps) as a brown solid. MS (ESI) calculated for (C₁₋₅H₂₂N₂O₄S) [M+H]⁺, 327.1; found, 327.0.

### Step 3: (S)-2-(1-(tert-butoxycarbonyl)pyrrolidin-2-yl)thiazole-4-carboxylic acid

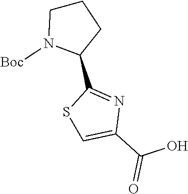 [SMILES: CC(C)(C)OC(=O)N1CCC[C@H]1c1nc(C(=O)O)cs1]

To a mixture of ethyl (S)-2-(1-(tert-butoxycarbonyl)pyrrolidin-2-yl)thiazole-4-carboxylate (51.5 g, 0.16 mol) in THF (300 mL) and water (200 mL) was added a solution of lithium hydroxide hydrate (26.5 g, 0.63 mol) in water (100 mL) dropwise at 0° C. The resulting mixture was stirred at 0° C. for 5 h. The organic layer was removed under vacuum. The residue was diluted with 200 mL of water and the pH value was adjusted to 3 by HCl (6 N). The solution was extracted with ethyl acetate. The combined organic layer was washed with brine, dried over anhydrous sodium sulfate and concentrated under vacuum to afford (S)-2-(1-(tert-butoxycarbonyl)pyrrolidin-2-yl)thiazole-4-carboxylic acid (45.0 g, 95%) as a light brown solid. MS (ESI) calculated for (C₁₃H₁₈N₂O₄S) [M−H], 297.1; found, 297.0.

### Step 4: tert-butyl (S)-2-(4-(methoxy(methyl)carbamoyl)thiazol-2-yl)pyrrolidine-1-carboxylate

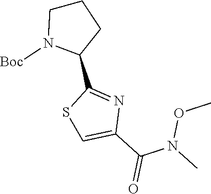 [SMILES: CON(C)C(=O)c1csc([C@@H]2CCCN2C(=O)OC(C)(C)C)n1]

A mixture of (S)-2-(1-(tert-butoxycarbonyl)pyrrolidin-2-yl)thiazole-4-carboxylic acid (90.0 g, 0.30 mol), methoxy(methyl)amine hydrogen chloride (43.6 g, 0.45 mol), HATU (114.0 g, 0.30 mol) and DIEA (96.7 g, 0.75 mol) in DMF (500 mL) was stirred at room temperature for 16 h. The mixture was diluted with water and the aqueous phase was extracted with ethyl acetate. The combined organic layer was washed with brine, dried over anhydrous sodium sulfate and concentrated under vacuum. The crude residue was purified by flash column chromatography with 40-80% ethyl acetate in petroleum ether to afford tert-butyl (S)-2-(4-(methoxy(methyl)carbamoyl)thiazol-2-yl)pyrrolidine-1-carboxylate (60.0 g, 59%) as a light yellow oil. MS (ESI) calculated for (C₁₋₅H₂₃N₃O₄S) [M+H]⁺, 342.1; found, 342.0.

### Step 5: tert-butyl (S)-2-(4-(3-methoxybenzoyl)thiazol-2-yl)pyrrolidine-1-carboxylate

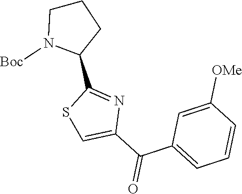 [SMILES: COc1cccc(C(=O)c2csc([C@@H]3CCCN3C(=O)OC(C)(C)C)n2)c1]

To a solution of tert-butyl (S)-2-(4-(methoxy(methyl)carbamoyl)thiazol-2-yl)pyrrolidine-1-carboxylate (30.0 g, 88.0 mmol) in anhydrous THF (300 mL) was added (3-methoxyphenyl)magnesium bromide (1M in THF, 530 mL, 0.53 mol) dropwise at −55° C. under nitrogen. The resulting mixture was stirred for 4 h below −20° C. The reaction was then quenched by the addition of saturated NH₄Cl aqueous solution at 0° C. cautiously. The mixture was extracted with ethyl acetate. The combined organic layer was washed with brine, dried over anhydrous sodium sulfate and concentrated under vacuum. The crude residue was purified by flash column chromatography with 10˜50% ethyl acetate in petroleum ether to afford tert-butyl (S)-2-(4-(3-methoxybenzoyl)thiazol-2-yl)pyrrolidine-1-carboxylate (24 g, 70%) as light yellow oil. MS (ESI) calculated for (C₂₀H₂₄N₂O₄S) [M+H]⁺, 389.1; found, 389.0.

### Step 6: (S)-(3-methoxyphenyl)(2-(pyrrolidin-2-yl)thiazol-4-yl)methanone HCl salt

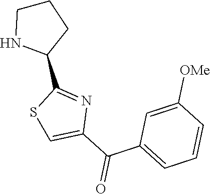 [SMILES: COc1cccc(C(=O)c2csc([C@@H]3CCCN3)n2)c1]

A mixture of tert-butyl (S)-2-(4-(3-methoxybenzoyl)thiazol-2-yl)pyrrolidine-1-carboxylate (24 g, 61.8 mmol) in HCl (4 M in dioxane, 200 mL) was stirred at room temperature for 2 h. The solvent was removed under vacuum to afford (S)-(3-methoxyphenyl)(2-(pyrrolidin-2-yl)thiazol-4-yl)methanone HCl salt (26 g, crude) as yellow oil, which was used for the next step without further purification. MS (ESI) calculated for (C₂₀H₁₆N₂O₂S) [M+H]⁺, 289.1; found, 289.0.

### Step 7: tert-butyl ((S)-1-(((S)-1-cyclohexyl-2-((S)-2-(4-(3-methoxybenzoyl)thiazol-2-yl)pyrrolidin-1-yl)-2-oxoethyl)amino)-1-oxopropan-2-yl)(methyl)carbamate

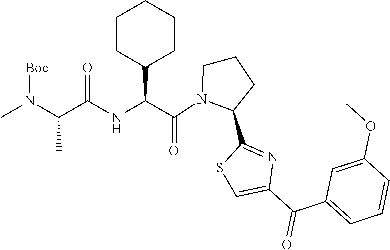 [SMILES: COc1cccc(C(=O)c2csc([C@@H]3CCCN3C(=O)[C@@H](NC(=O)[C@H](C)N(C)C(=O)OC(C)(C)C)C3CCCCC3)n2)c1]

To a solution of 4-[(3-methoxyphenyl)carbonyl]-2-[(2S)-pyrrolidin-2-yl]-1,3-thiazole (25 g, 86.70 mmol) and (2S)-2-[(2S)-2-[[(tert-butoxy)carbonyl](methyl)amino]propanamido]-2-cyclohexylacetic acid (29.7 g, 86.73 mmol) in ethyl acetate (400 mL) were added 4-(4,6-dmethoxy-1,3,5-triazine-2-yl)-4-methyl morpholinium chloride (DMT-MM) (26.35 g, 95.47 mmol) and 4-methylmorpholine (21.9 g, 216.83 mmol) at 0° C. The resulting mixture was stirred at room temperature for 3 h. The reaction was then quenched by the addition of water and the aqueous phase was extracted with ethyl acetate. The combined organic layer was washed with brine, dried over anhydrous sodium sulfate and concentrated under vacuum. The crude residue was purified by flash column chromatography with 0-30% ethyl acetate in petroleum ether to afford tert-butyl ((S)-1-(((S)-1-cyclohexyl-2-((S)-2-(4-(3-methoxybenzoyl)thiazol-2-yl)pyrrolidin-1-yl)-2-oxoethyl)amino)-1-oxopropan-2-yl)(methyl)carbamate (24 g, 46%) as light yellow oil. MS (ESI) calculated for (C₃₂H₄₄N₄O₆S) [M+H]⁺, 613.3; found, 613.0.

### Step 8: (S)—N—((S)-1-cyclohexyl-2-((S)-2-(4-(3-hydroxybenzoyl)thiazol-2-yl)pyrrolidin-1-yl)-2-oxoethyl)-2-(methylamino)propanamide

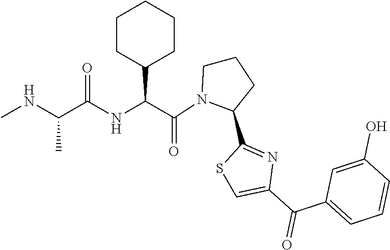 [SMILES: CN[C@@H](C)C(=O)N[C@H](C(=O)N1CCC[C@H]1c1nc(C(=O)c2cccc(O)c2)cs1)C1CCCCC1]

To a solution of tert-butyl ((S)-1-(((S)-1-cyclohexyl-2-((S)-2-(4-(3-methoxybenzoyl)thiazol-2-yl)pyrrolidin-1-yl)-2-oxoethyl)amino)-1-oxopropan-2-yl)(methyl)carbamate (9.0 g, 14.69 mmol) in dichloromethane (120 mL) was added BBr₃ (10.9 g, 44.1 mmol) dropwise at −78° C. The resulting mixture was stirred below 0° C. for 4 h under nitrogen. The reaction was then quenched by the addition of water cautiously and the aqueous phase was extracted with dichloromethane. The combined organic layer was washed with brine, dried over anhydrous sodium sulfate and concentrated under vacuum to afford (S)—N—((S)-1-cyclohexyl-2-((S)-2-(4-(3-hydroxybenzoyl)thiazol-2-yl)pyrrolidin-1-yl)-2-oxoethyl)-2-(methylamino)propanamide (9 g, crude) as light brown oil, which was used for the next step without further purification. MS (ESI) calculated for (C₂₆H₃₄N₄O₄S) [M+H]⁺, 499.2; found, 499.0.

### Step 9: tert-butyl ((S)-1-(((S)-1-cyclohexyl-2-((S)-2-(4-(3-hydroxybenzoyl)thiazol-2-yl)pyrrolidin-1-yl)-2-oxoethyl)amino)-1-oxopropan-2-yl)(methyl)carbamate

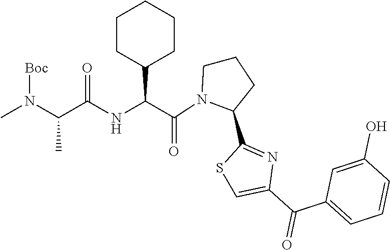 [SMILES: C[C@@H](C(=O)N[C@H](C(=O)N1CCC[C@H]1c1nc(C(=O)c2cccc(O)c2)cs1)C1CCCCC1)N(C)C(=O)OC(C)(C)C]

To a solution of (S)—N—((S)-1-cyclohexyl-2-((S)-2-(4-(3-hydroxybenzoyl)thiazol-2-yl)pyrrolidin-1-yl)-2-oxoethyl)-2-(methylamino)propanamide (10 g, 20.05 mmol) and sodium bicarbonate (3.6 g, 43.21 mmol) in dioxane (120 mL) was added a solution of Boc₂O (5.6 g, 25.48 mmol) in dioxane (30 mL) dropwise at 0° C. The mixture was stirred at room temperature for 2 h. The reaction was diluted with water and the aqueous phase was extracted with ethyl acetate. The combined organic layer was washed with brine, dried over anhydrous sodium sulfate and concentrated under vacuum. The crude residue was purified by flash column chromatography with 10˜50% ethyl acetate in petroleum ether to afford tert-butyl ((S)-1-(((S)-1-cyclohexyl-2-((S)-2-(4-(3-hydroxybenzoyl)thiazol-2-yl)pyrrolidin-1-yl)-2-oxoethyl)amino)-1-oxopropan-2-yl)(methyl)carbamate (5.2 g, 59% over 2 steps) as light yellow oil. MS (ESI) calculated for (C₃₁H₄₂N₄O₆S) [M+H]⁺, 599.3; found, 599.3. ¹H NMR (300 MHz, Chloroform-d) δ 8.60 (br, 1H), 8.09 (d, J=2.0 Hz, 1H), 7.78-7.54 (m, 2H), 7.38-7.34 (m, 1H), 7.11-7.08 (m, 1H), 6.79 (br, 1H), 5.68-5.47 (m, 1H), 4.85-4.64 (m, 2H), 4.00-3.59 (m, 2H), 2.80 (s, 3H), 2.58-2.09 (m, 4H), 1.87-1.58 (m, 6H), 1.50 (s, 9H), 1.36 (d, J=7.1 Hz, 3H), 1.18-0.81 (m, 5H).

### Example 76: Synthesis of 3-(2-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)ethoxy)propanoic acid

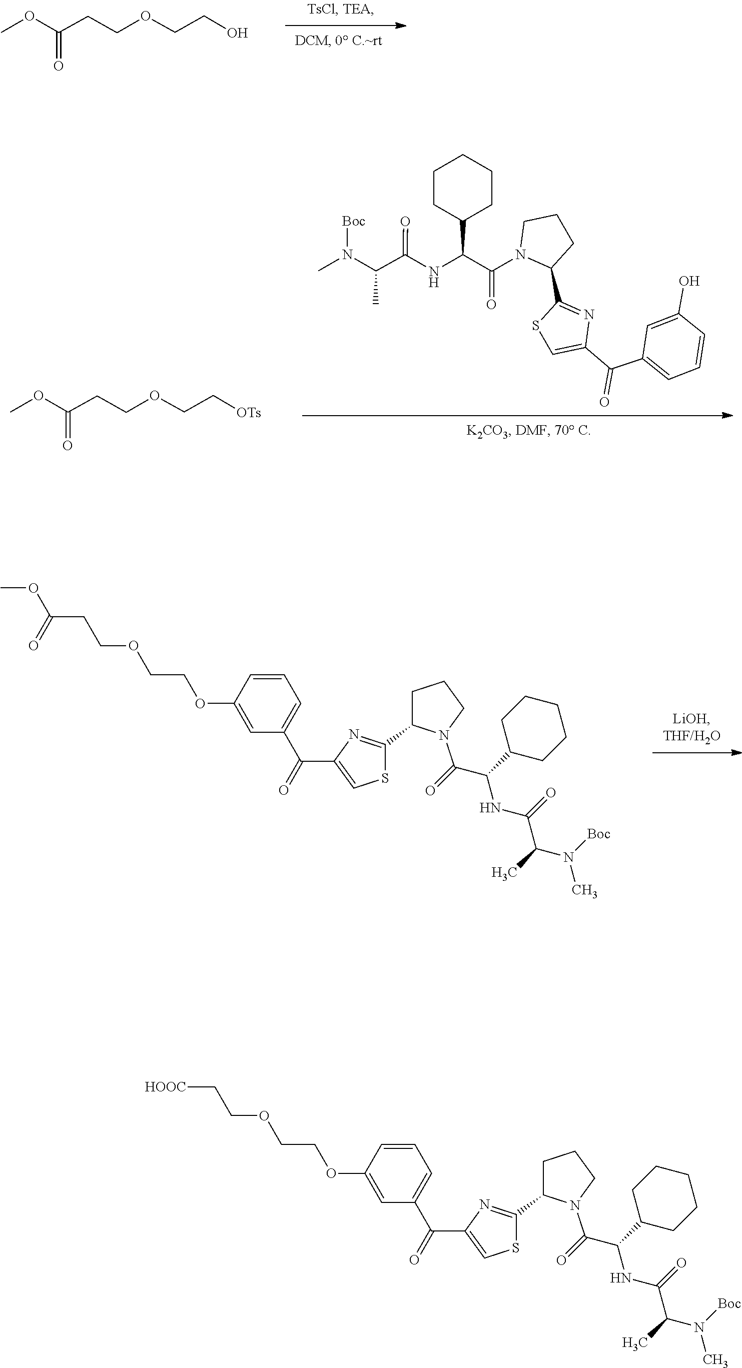

### Step 1: methyl 3-[2-[(4-methylbenzenesulfonyl)oxy]ethoxy]propanoate

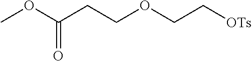 [SMILES: COC(=O)CCOCCOS(=O)(=O)c1ccc(C)cc1]

To a solution of methyl 3-(2-hydroxyethoxy)propanoate (1.00 g, 6.7 mmol) in dichloromethane (15 mL) was added triethylamine (1.72 g, 13.2 mmol) and p-TsCl (1.54 g, 8.1 mmol) at room temperature. The mixture was stirred at room temperature for 16 h. The reaction mixture was quenched by the addition of water and the aqueous phase was extracted with dichloromethane. The combined organic layer was washed with brine, dried over anhydrous sodium sulfate, filtered and concentrated under vacuum. The residue was purified by flash column chromatography with 0-50% ethyl acetate in petroleum ether to afford methyl 3-[2-[(4-methylbenzenesulfonyl)oxy]ethoxy]propanoate (1.05 g, 51%) as yellow oil. MS (ESI) calculated for (C₁₃H₁₈O₆S) [M+H]⁺, 303.1; found, 303.0.

### Step 2: methyl 3-(2-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)ethoxy)propanoate

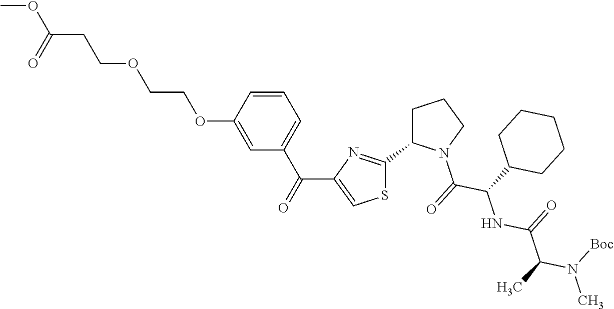 [SMILES: COC(=O)CCOCCOc1cccc(C(=O)c2csc([C@@H]3CCCN3C(=O)[C@@H](NC(=O)[C@H](C)N(C)C(=O)OC(C)(C)C)C3CCCCC3)n2)c1]

To a solution of methyl 3-[2-[(4-methylbenzenesulfonyl)oxy]ethoxy]propanoate (1.05 g, 3.5 mmol) in N,N-dimethylformamide (10 mL) was added tert-butyl ((S)-1-(((S)-1-cyclohexyl-2-((S)-2-(4-(3-hydroxybenzoyl)thiazol-2-yl)pyrrolidin-1-yl)-2-oxoethyl)amino)-1-oxopropan-2-yl)(methyl)carbamate (1.32 g, 2.2 mmol) and potassium carbonate (607 mg, 4.4 mmol). The mixture was stirred at 70° C. for 16 h. After cooled to room temperature, the reaction mixture was diluted with water and the aqueous phase was extracted with ethyl acetate. The combined organic layer was washed with brine, dried over anhydrous sodium sulfate, filtered and concentrated under vacuum. The residue was purified by flash column chromatography with 0˜50% ethyl acetate in petroleum ether to afford methyl 3-(2-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)ethoxy)propanoate (1.0 g, 62%) as light yellow oil. MS (ESI) calculated for (C₃₆H₅₀N₄O₉S) [M+H]⁺, 715.3; found, 715.0.

### Step 3: 3-(2-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)ethoxy)propanoic acid

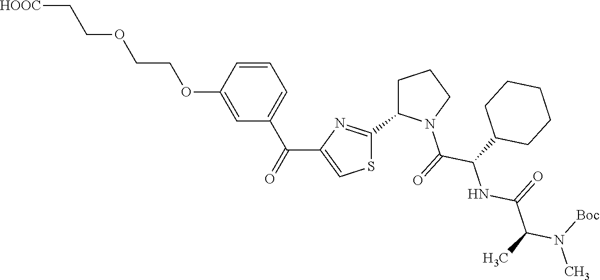 [SMILES: C[C@@H](C(=O)N[C@H](C(=O)N1CCC[C@H]1c1nc(C(=O)c2cccc(OCCOCCC(=O)O)c2)cs1)C1CCCCC1)N(C)C(=O)OC(C)(C)C]

To a solution of 3-(2-(3-(2-((S)-1-((S)-2-((S)-2-((tert-butoxycarbonyl)(methyl)amino) propanamido)-2-cyclohexylacetyl)pyrrolidin-2-yl)thiazole-4-carbonyl)phenoxy)ethoxy) propanoic acid (1.0 g, 1.37 mmol) in tetrahydrofuran (5 mL) and H₂O (5 mL) was added lithium hydroxide hydrate (115 mg, 2.75 mmol). The mixture was stirred at room temperature for 5 h. The reaction mixture was diluted with water and adjusted the pH to ˜3 by HCl (2 N). The mixture was extracted with ethyl acetate. The combined organic layer was washed with brine, dried over anhydrous sodium sulfate and concentrated under vacuum. The crude residue was purified by reverse phase flash column chromatography with 5˜55% acetonitrile in water to afford 3-(2-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)ethoxy)propanoic acid (733.9 mg, 75%) as a white solid. ¹H NMR (300 MHz, Methanol-d4) δ 8.33 (s, 1H), 7.77-7.65 (m, 2H), 7.52-7.39 (m, 1H), 7.26-7.24 (m, 1H), 5.70-5.46 (m, 1H), 4.71-4.42 (m, 2H), 4.28-4.16 (m, 2H), 4.05-3.72 (m, 6H), 2.80 (s, 3H), 2.49 (t, J=7.2 Hz, 2H), 2.44-2.03 (m, 4H), 1.89-1.55 (m, 6H), 1.49 (s, 9H), 1.37-1.35 (m, 3H), 1.30-0.95 (m, 5H). MS (ESI) calculated for (C₃₆H₅₀N₄O₉S) [M+H]⁺, 715.3; found, 715.5.

### Preparation of 3-(2-(2-(2-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)ethoxy)ethoxy)ethoxy)propanoic acid

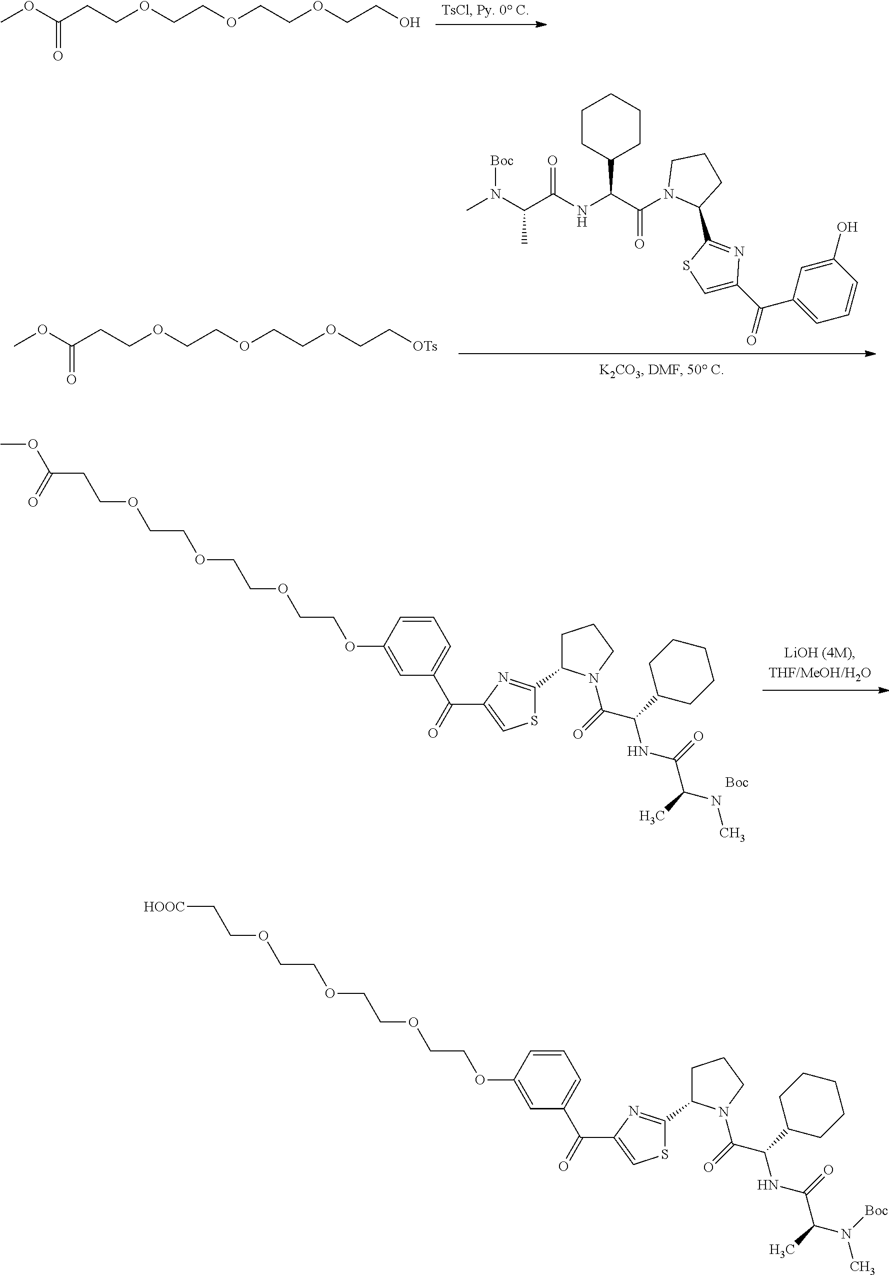

### Step 1: methyl 3-(2-(2-(2-(tosyloxy)ethoxy)ethoxy)ethoxy)propanoate

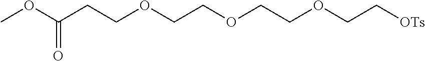 [SMILES: COC(=O)CCOCCOCCOCCOS(=O)(=O)c1ccc(C)cc1]

To a solution of methyl 3-(2-(2-(2-hydroxyethoxy)ethoxy)ethoxy)propanoate (1 g, 4.23 mmol) in pyridine (10 mL) was added p-TsCl (1.2 g, 6.29 mmol). The mixture was stirred at room temperature for 16 h. The reaction mixture was diluted with water and the aqueous phase was extracted with ethyl acetate. The combined organic layer was washed with brine, dried over anhydrous sodium sulfate, filtered and concentrated under vacuum. The residue was purified by flash column chromatography with 0-50% ethyl acetate in petroleum ether to afford methyl 3-(2-(2-(2-(tosyloxy)ethoxy)ethoxy)ethoxy)propanoate (1.0 g, 60%) as light yellow oil. MS (ESI) calculated for (C₁₇H₂₆O₈S) [M+H]⁺, 391.1; found, 391.0.

### Step 2: methyl 3-(2-(2-(2-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)ethoxy)ethoxy)ethoxy)propanoate

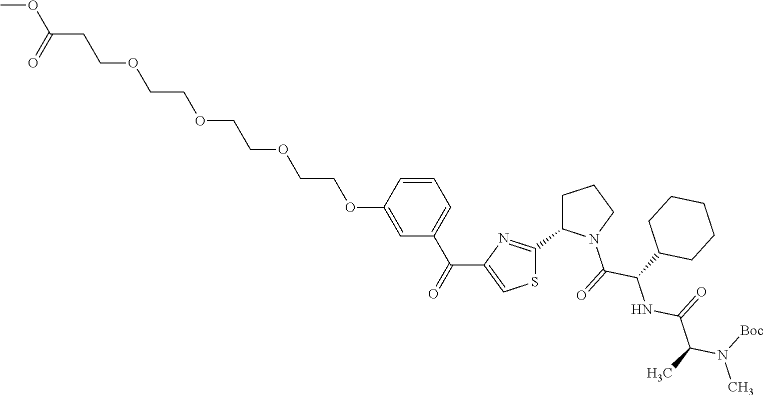 [SMILES: COC(=O)CCOCCOCCOCCOc1cccc(C(=O)c2csc([C@@H]3CCCN3C(=O)[C@@H](NC(=O)[C@H](C)N(C)C(=O)OC(C)(C)C)C3CCCCC3)n2)c1]

To a solution of methyl 3-(2-(2-(2-(tosyloxy)ethoxy)ethoxy)ethoxy)propanoate (1.0 g, 2.56 mmol) in N,N-dimethylformamide (10 mL) was added tert-butyl ((S)-1-(((S)-1-cyclohexyl-2-((S)-2-(4-(3-hydroxybenzoyl)thiazol-2-yl)pyrrolidin-1-yl)-2-oxoethyl)amino)-1-oxopropan-2-yl)(methyl)carbamate (1.2 g, 2.00 mmol) and potassium carbonate (400 mg, 2.89 mmol). The mixture was stirred at 50° C. for 16 h. The reaction mixture was then diluted with water and the aqueous phase was extracted with ethyl acetate. The combined organic layer was washed with brine, dried over anhydrous sodium sulfate, filtered and concentrated under vacuum. The crude residue was purified by flash column chromatography with 10˜60% ethyl acetate in petroleum ether to afford methyl 3-(2-(2-(2-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)ethoxy)ethoxy)ethoxy)propanoate (1.0 g, 61%) as a light yellow solid. MS (ESI) calculated for (C₄₁H₆₀N₄O₁₁S) [M+H]⁺, 817.4; found, 817.0.

### Step 3: 3-(2-(2-(2-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)ethoxy)ethoxy)ethoxy)propanoic acid

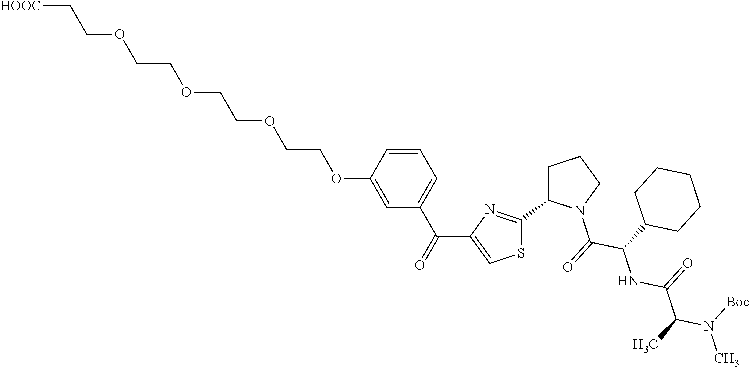 [SMILES: C[C@@H](C(=O)N[C@H](C(=O)N1CCC[C@H]1c1nc(C(=O)c2cccc(OCCOCCOCCOCCC(=O)O)c2)cs1)C1CCCCC1)N(C)C(=O)OC(C)(C)C]

To a solution of methyl 3-(2-[2-[2-(3-[2-[(2S)-1-[(2S)-2-[(2S)-2-[[(tert-butoxy)carbonyl](methyl)amino]propanamido]-2-cyclohexylacetyl]pyrrolidin-2-yl]-1,3-thiazole-4-carbonyl]phenoxy)ethoxy]ethoxy]ethoxy)propanoate (1.0 g, 1.22 mmol) in tetrahydrofuran (4 mL) and MeOH (4 mL) was added lithium hydroxide solution (4 M, 2 mL). The resulting mixture was stirred at room temperature for 6 h. The mixture was diluted with water and adjusted the pH to ˜3 by HCl (2 N). The aqueous phase was extracted with ethyl acetate. The combined organic layer was dried over anhydrous sodium sulfate, filtered and concentrated under vacuum. The residue was purified by flash column chromatography with 10˜80% acetonitrile in water to afford 3-(2-(2-(2-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)ethoxy)ethoxy)ethoxy)propanoic acid (573.3 mg, 58%) as a white solid. ¹H NMR (300 MHz, Methanol-d4) δ 8.35 (s, 1H), 7.79-7.71 (m, 2H), 7.47-7.44 (m, 1H), 7.28-7.26 (m, 1H), 5.73-5.46 (m, 1H), 4.69-4.35 (m, 2H), 4.28-4.18 (m, 2H), 4.05-3.86 (m, 4H), 3.78-3.56 (m, 10H), 2.88 (s, 3H), 2.54 (t, J=6.3 Hz, 2H), 2.47-2.11 (m, 4H), 1.86-1.53 (m, 6H), 1.48 (s, 9H), 1.36 (d, J=7.2 Hz, 3H), 1.28-0.99 (m, 5H). MS (ESI) calculated for (C₄₀H₅₈N₄O₁₁S) [M+H]⁺, 803.4; found, 803.7.

### Example 77: Synthesis of 1-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)-3,6,9,12-tetraoxapentadecan-15-oic acid

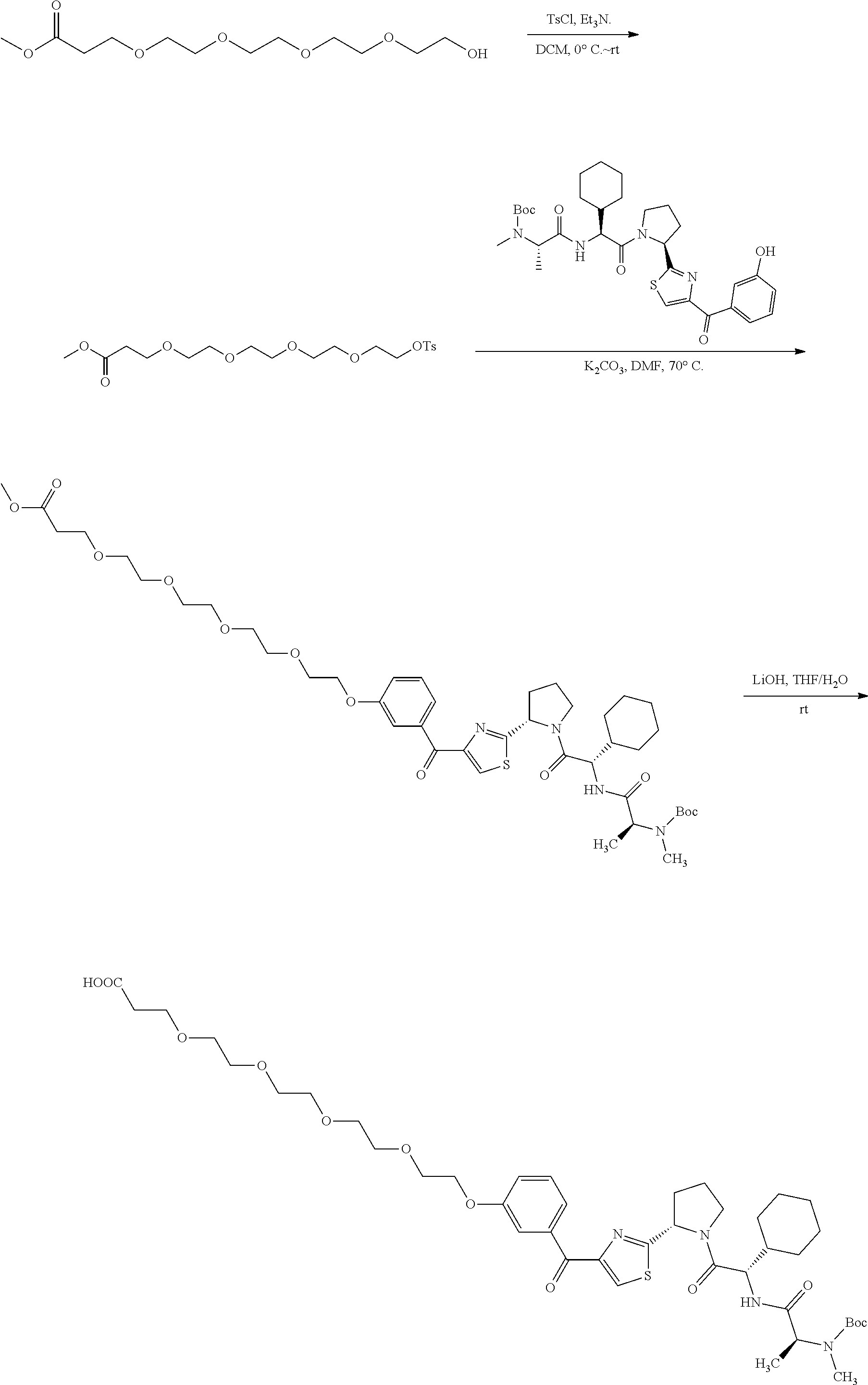 [SMILES: COC(=O)CCOCCOCCOCCOCCO ; C[C@@H](C(=O)N[C@H](C(=O)N1CCC[C@H]1c1nc(C(=O)c2cccc(O)c2)cs1)C1CCCCC1)N(C)C(=O)OC(C)(C)C ; COC(=O)CCOCCOCCOCCOCCOS(=O)(=O)c1ccc(C)cc1 ; COC(=O)CCOCCOCCOCCOCCOc1cccc(C(=O)c2csc([C@@H]3CCCN3C(=O)[C@@H](NC(=O)[C@H](C)N(C)C(=O)OC(C)(C)C)C3CCCCC3)n2)c1 ; C[C@@H](C(=O)N[C@H](C(=O)N1CCC[C@H]1c1nc(C(=O)c2cccc(OCCOCCOCCOCCOCCC(=O)O)c2)cs1)C1CCCCC1)N(C)C(=O)OC(C)(C)C]

### Step 1: methyl 1-[(4-methylbenzenesulfonyl)oxy]-3,6,9,12-tetraoxapentadecan-15-oate

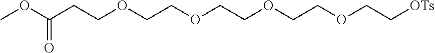 [SMILES: COC(=O)CCOCCOCCOCCOCCOS(=O)(=O)c1ccc(C)cc1]

To a solution of methyl 1-hydroxy-3,6,9,12-tetraoxapentadecan-15-oate (970 mg, 3.46 mmol) in dichloromethane (20 mL) was added triethylamine (700 mg, 6.93 mmol) and p-TsCl (990 mg, 5.19 mmol). The mixture was stirred at room temperature for 16 h. The reaction mixture was diluted with water and the aqueous phase was extracted with ethyl acetate. The combined organic layer was washed with brine, dried over anhydrous sodium sulfate, filtered and concentrated under vacuum. The residue was purified by flash column chromatography with 0-50% ethyl acetate in petroleum ether to afford methyl 1-[(4-methylbenzenesulfonyl)oxy]-3,6,9,12-tetraoxapentadecan-15-oate (1.28 g, 85%) as light yellow oil. MS (ESI) calculated for (C₁₉H₃₀O₉S) [M+H]⁺, 435.2; found, 435.0.

### Step 2: methyl 1-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)-3,6,9,12-tetraoxapentadecan-15-oate

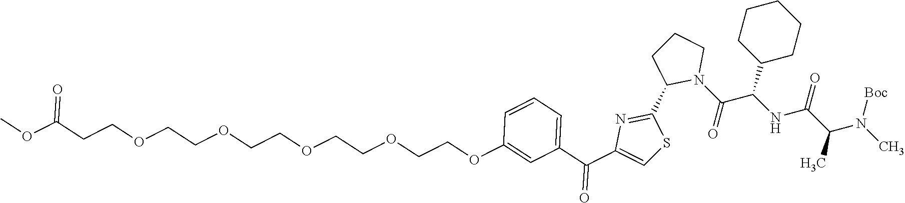

To a solution of methyl 1-[(4-methylbenzenesulfonyl)oxy]-3,6,9,12-tetraoxapentadecan-15-oate (1.28 g, 2.95 mmol) in N,N-dimethylformamide (10 mL) was added tert-butyl ((S)-1-(((S)-1-cyclohexyl-2-((S)-2-(4-(3-hydroxybenzoyl)thiazol-2-yl)pyrrolidin-1-yl)-2-oxoethyl)amino)-1-oxopropan-2-yl)(methyl)carbamate (1.17 g, 1.96 mmol) and potassium carbonate (370 mg, 2.68 mmol). The mixture was stirred at 50° C. for 16 h. The reaction mixture was then diluted with water and the aqueous phase was extracted with ethyl acetate. The combined organic layer was washed with brine, dried over anhydrous sodium sulfate, filtered and concentrated under vacuum. The residue was purified by flash column chromatography with 0˜50% ethyl acetate in petroleum ether to afford methyl 1-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)-3,6,9,12-tetraoxapentadecan-15-oate (1.2 g, 60%) as light yellow oil. MS (ESI) calculated for (C₄₃H₆₄N₄O₁₂S) [M+H]⁺, 861.4; found, 861.0.

### Step 3: 1-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)-3,6,9,12-tetraoxapentadecan-15-oic acid

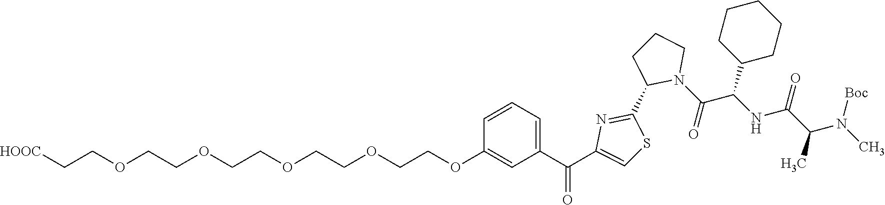 [SMILES: C[C@@H](C(=O)N[C@H](C(=O)N1CCC[C@H]1c1nc(C(=O)c2cccc(OCCOCCOCCOCCOCCC(=O)O)c2)cs1)C1CCCCC1)N(C)C(=O)OC(C)(C)C]

To a solution of 1-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)-3,6,9,12-tetraoxapentadecan-15-oate (1.2 g, 1.39 mmol) in tetrahydrofuran (10 mL) and H₂O (10 mL) was added lithium hydroxide hydrate (140 mg, 3.33 mmol). The mixture was stirred at room temperature for 16 h. The reaction mixture was diluted with water and the pH was adjusted to ˜3 by HCl (2 N). The aqueous phase was extracted with ethyl acetate. The combined organic layer was washed with brine, dried over anhydrous sodium sulfate, filtered and concentrated under vacuum. The residue was purified by flash column chromatography with 10-80% acetonitrile in water to afford 1-(3-(2-((S)-1-((S)-2-((S)-2-((tert-butoxycarbonyl)(methyl)amino)propanamido)-2-cyclohexylacetyl)pyrrolidin-2-yl)thiazole-4-carbonyl)phenoxy)-3,6,9,12-tetraoxapentadecan-15-oic acid (805.2 mg, 68%) as a white solid. ¹H NMR (300 MHz, Methanol-d₄) δ 8.35 (s, 1H), 7.80-7.69 (m, 2H), 7.50-7.42 (m, 1H), 7.26-7.23 (m, 1H), 5.50-5.45 (m, 1H), 4.56-4.53 (m, 2H), 4.32-4.16 (m, 2H), 4.05-3.85 (m, 4H), 3.77-3.66 (m, 6H), 3.66-3.56 (m, 8H), 2.80 (s, 3H), 2.45 (t, J=7.2 Hz, 2H), 2.41-2.08 (m, 3H), 1.88-1.55 (m, 6H), 1.49 (s, 9H), 1.36 (d, J=7.2 Hz, 3H), 1.08-1.04 (m, 6H). MS (ESI) calculated for (C₄₂H₆₂N₄O₁₂S) [M+H]⁺, 847.4; found, 847.8.

### Example 78: 16-(((3S,5S)-1-((S)-2-((S)-2-((tert-butoxycarbonyl)(methyl)amino)propanamido)-2-cyclohexylacetyl)-5-(((R)-1,2,3,4-tetrahydronaphthalen-1-yl)carbamoyl)pyrrolidin-3-yl)amino)-16-oxo-4,7,10,13-tetraoxahexadecanoic acid

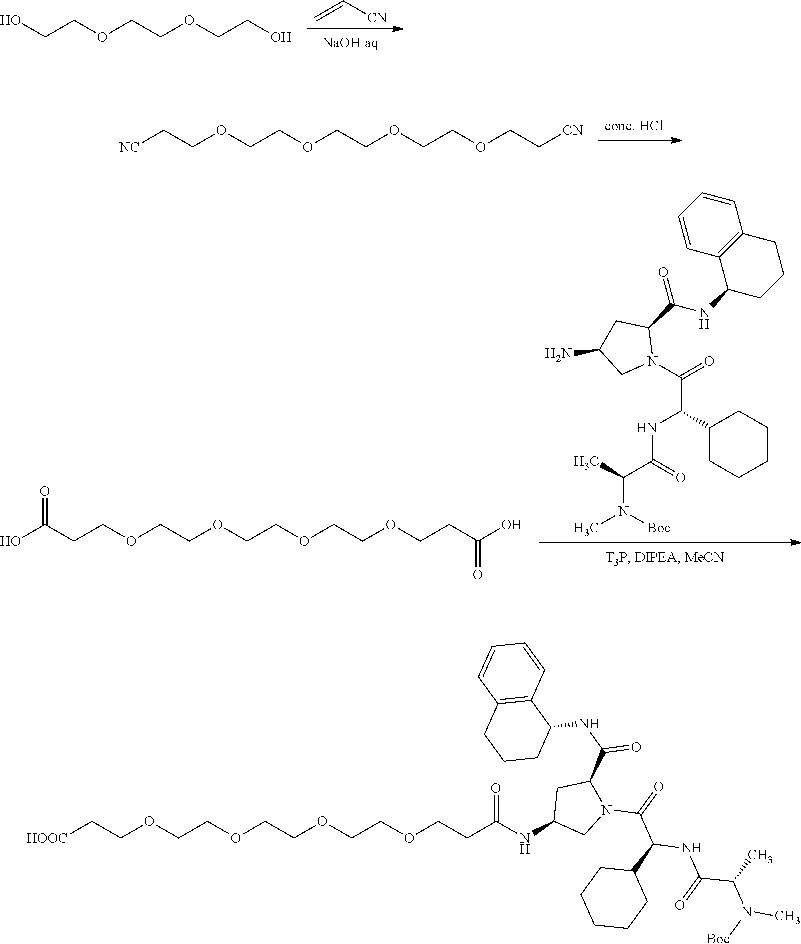

### Step 1: 4,7,10,13-tetraoxahexadecanedinitrile

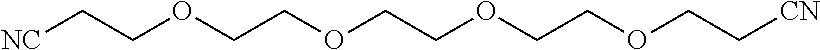 [SMILES: N#CCCOCCOCCOCCOCCC#N]

To a stirred solution of 2,2′-(ethane-1,2-diylbis(oxy))bis(ethan-1-ol) (15 g, 99.90 mmol) and NaOH aqueous (1.2 mL, 40% wt) was added acrylonitrile (12.2 g, 230 mmol) dropwise at 0° C. The solution was stirred at 30° C. for 16 h. When the reaction was complete, the reaction was quenched by the addition of 100 mL H₂O, neutralized to pH 7 by HCl (1 N). The aqueous solution was extracted with ethyl acetate (100 mL×3). The combined organic solution was dried over Na₂SO₄ and concentrated to afford 4,7,10,13-tetraoxahexadecanedinitrile (15 g, 59%) as yellow oil. ¹H NMR (300 MHz, Chloroform-d) δ 3.72 (t, J=6.3 Hz, 4H), 3.69-3.62 (m, 12H), 2.62 (t, J=6.3 Hz, 4H).

### Step 2: 4,7,10,13-tetraoxahexadecanedioic acid

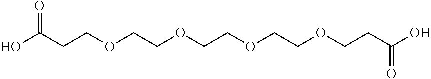 [SMILES: O=C(O)CCOCCOCCOCCOCCC(=O)O]

Concentrated HCl (68 mL) was added to 4,7,10,13-tetraoxahexadecanedinitrile (15 g, 58.60 mmol). The solution was stirred at 70° C. overnight. When the reaction was completed, the mixture was filtered, and the filtrate was concentrated to afford the residue, which was purified by flash column chromatography with 30˜100% ethyl acetate in petroleum ether to afford 4,7,10,13-tetraoxahexadecanedioic acid (10.0 g, crude) as yellow oil. ¹H NMR (400 MHz, DMSO-d₆) δ 12.18 (s, 2H), 3.60 (t, J=6.4 Hz, 4H), 3.51-3.48 (m, 12H), 2.44 (t, J=6.4 Hz, 4H).

### Step 3: 16-(((3S,5S)-1-((S)-2-((S)-2-((tert-butoxycarbonyl)(methyl)amino)propanamido)-2-cyclohexylacetyl)-5-(((R)-1,2,3,4-tetrahydronaphthalen-1-yl)carbamoyl)pyrrolidin-3-yl)amino)-16-oxo-4,7,10,13-tetraoxahexadecanoic acid

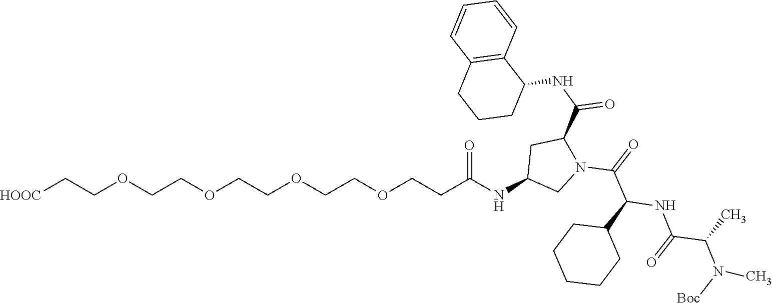 [SMILES: C[C@@H](C(=O)N[C@H](C(=O)N1C[C@@H](NC(=O)CCOCCOCCOCCOCCC(=O)O)C[C@H]1C(=O)N[C@@H]1CCCc2ccccc21)C1CCCCC1)N(C)C(=O)OC(C)(C)C]

To a stirred solution of tert-butyl ((S)-1-(((S)-2-((2S,4S)-4-amino-2-(((R)-1,2,3,4-tetrahydronaphthalen-1-yl)carbamoyl)pyrrolidin-1-yl)-1-cyclohexyl-2-oxoethyl)amino)-1-oxopropan-2-yl)(methyl)carbamate (1.5 g, 2.57 mmol), 4,7,10,13-tetraoxahexadecanedioic acid (5.0 g, 12.90 mmol) and DIEA (1.7 g, 12.86 mmol) in acetonitrile (30 mL) under nitrogen was added T₃P (6.5 g, 10.28 mmol). The solution was stirred at 20° C. for 16 h. When the reaction was completed, the reaction was quenched by the addition of 50 mL H₂O. The aqueous solution was extracted with ethyl acetate (30 mL×3). The combined organic solution was dried over Na₂SO₄ to give the residue which was purified by reverse phase FC with 5˜50% acetonitrile in H₂O to afford 16-(((3S,5S)-1-((S)-2-((S)-2-((tert-butoxycarbonyl)(methyl)amino)propanamido)-2-cyclohexylacetyl)-5-(((R)-1,2,3,4-tetrahydronaphthalen-1-yl)carbamoyl)pyrrolidin-3-yl)amino)-16-oxo-4,7,10,13-tetraoxahexadecanoic acid (511.2 mg, 23%) as white solid. ¹H NMR (400 MHz, Methanol-d₄) δ 7.44-7.38 (m, 1H), 7.21-7.12 (m, 2H), 7.12-7.06 (m, 1H), 5.06 (t, J=6.0 Hz, 1H), 4.67-4.38 (m, 4H), 4.22-4.18 (m, 1H), 3.74-3.70 (m, 4H), 3.68-3.59 (m, 11H), 3.55-3.50 (m, 1H), 2.91-2.73 (m, 5H), 2.60-2.41 (m, 5H), 1.93-1.83 (m, 6H), 1.82-1.66 (m, 5H), 1.49 (s, 9H), 1.39-0.98 (m, 9H). MS (ESI) calculated for (C₄₄H₆₉N₅O₁₂) [M+1]⁺, 860.7; found, 860.7.

### Example 79: General Procedures for CTM syntheses using coupling chemistry

General Coupling 1:

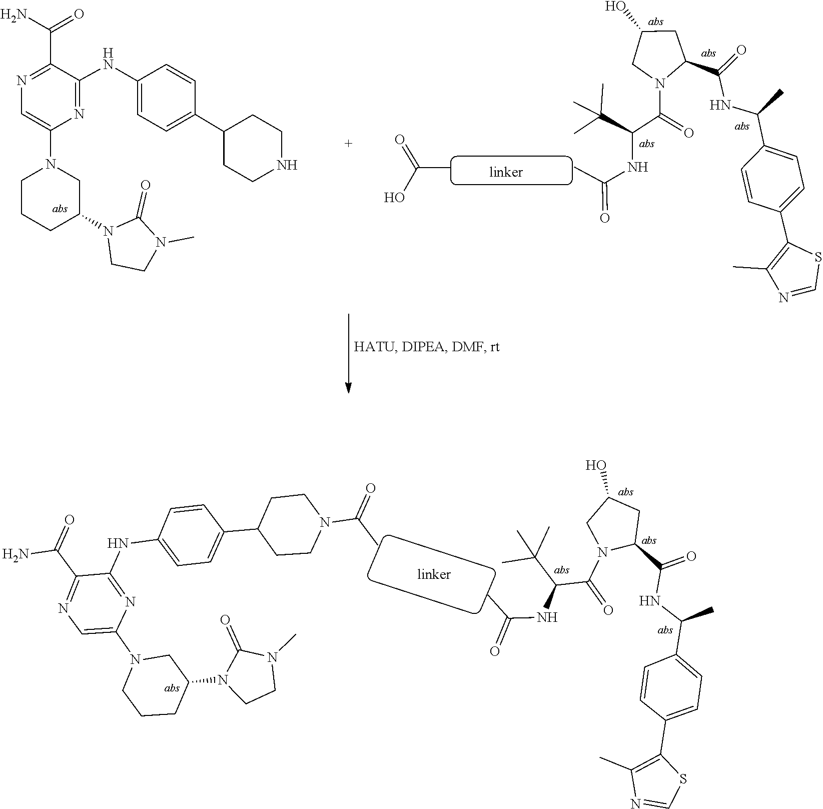

In a typical procedure, a mixture of amine (1 equiv), acid (1.1 equiv), HATU (1.2 equiv), DIPEA (3 equiv) and DMF (0.2 M) was allowed to stir at room temperature for 1 hour. The mixture was purified by HPLC (5-95% MeCN in H₂O with 0.1% TFA) to afford the desired product.

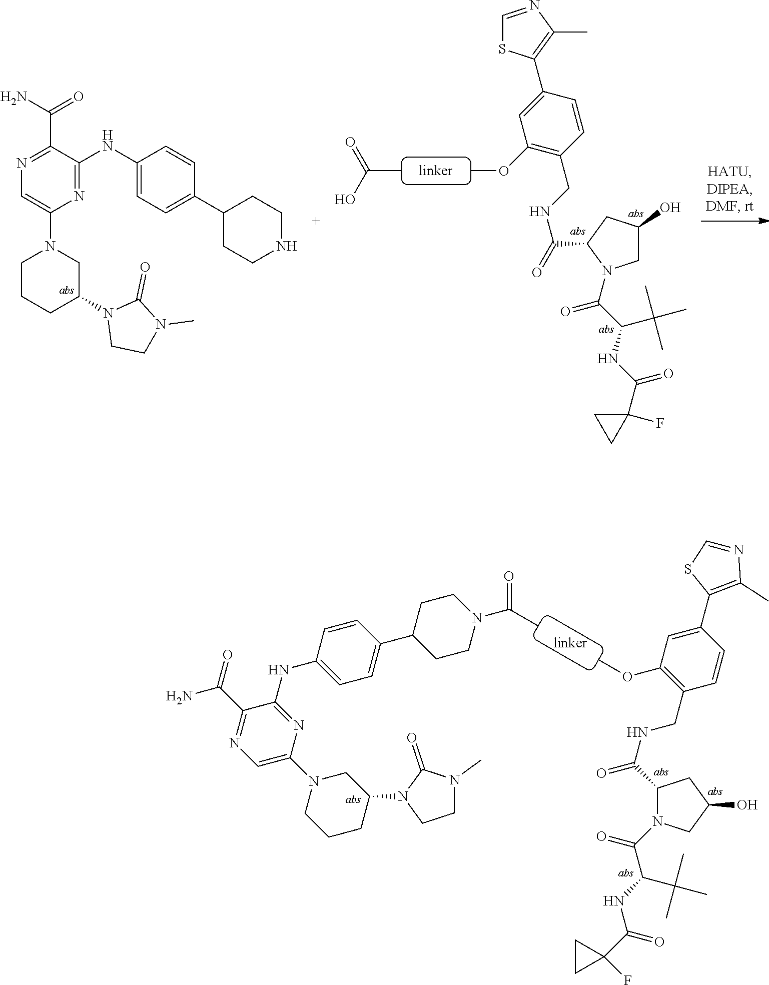

In a typical procedure, a mixture of amine (1 equiv), acid (1.1 equiv), HATU (1.2 equiv), DIPEA (3 equiv) and DMF (0.2 M) was allowed to stir at room temperature for 1 hour. The mixture was purified by HPLC (5-95% MeCN in H₂O with 0.1% TFA) to afford the desired product.

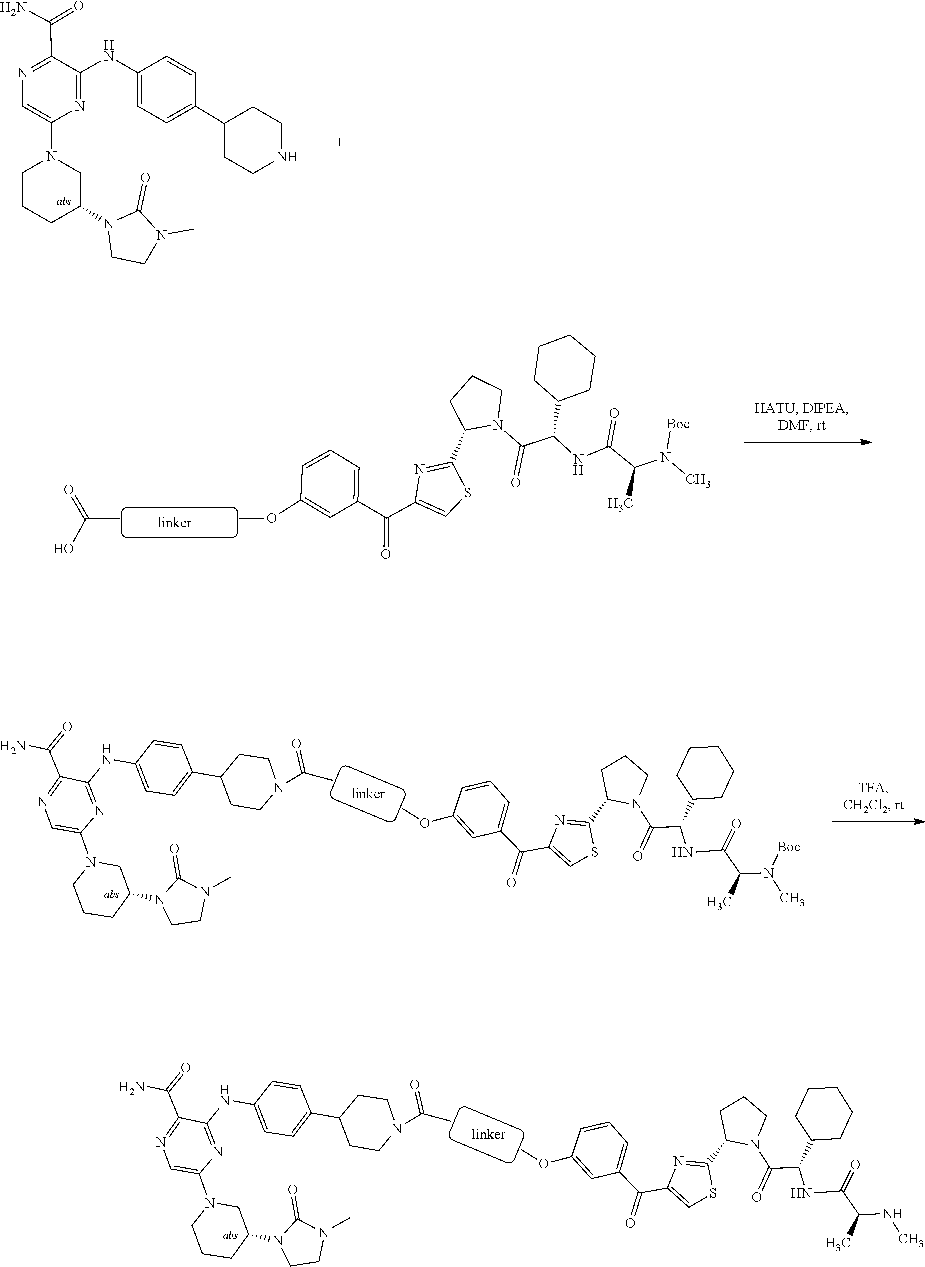

In a typical procedure, a mixture of amine (1 equiv), acid (1.1 equiv), HATU (1.2 equiv), DIPEA (3 equiv) and DMF (0.2 M) was allowed to stir at room temperature for 1 hour. EtOAc and H₂O were added. The organic layer was dried with MgSO₄, filtered, concentrated and carried to the next step.

TFA (20 equiv) and CH₂C₁₂ (0.1 M) were added and the mixture was allowed to stir at room temperature for 1 h. The volatiles were removed and the mixture was purified by HPLC (5-95% MeCN in H₂O with 0.1% TFA) to afford the desired product.

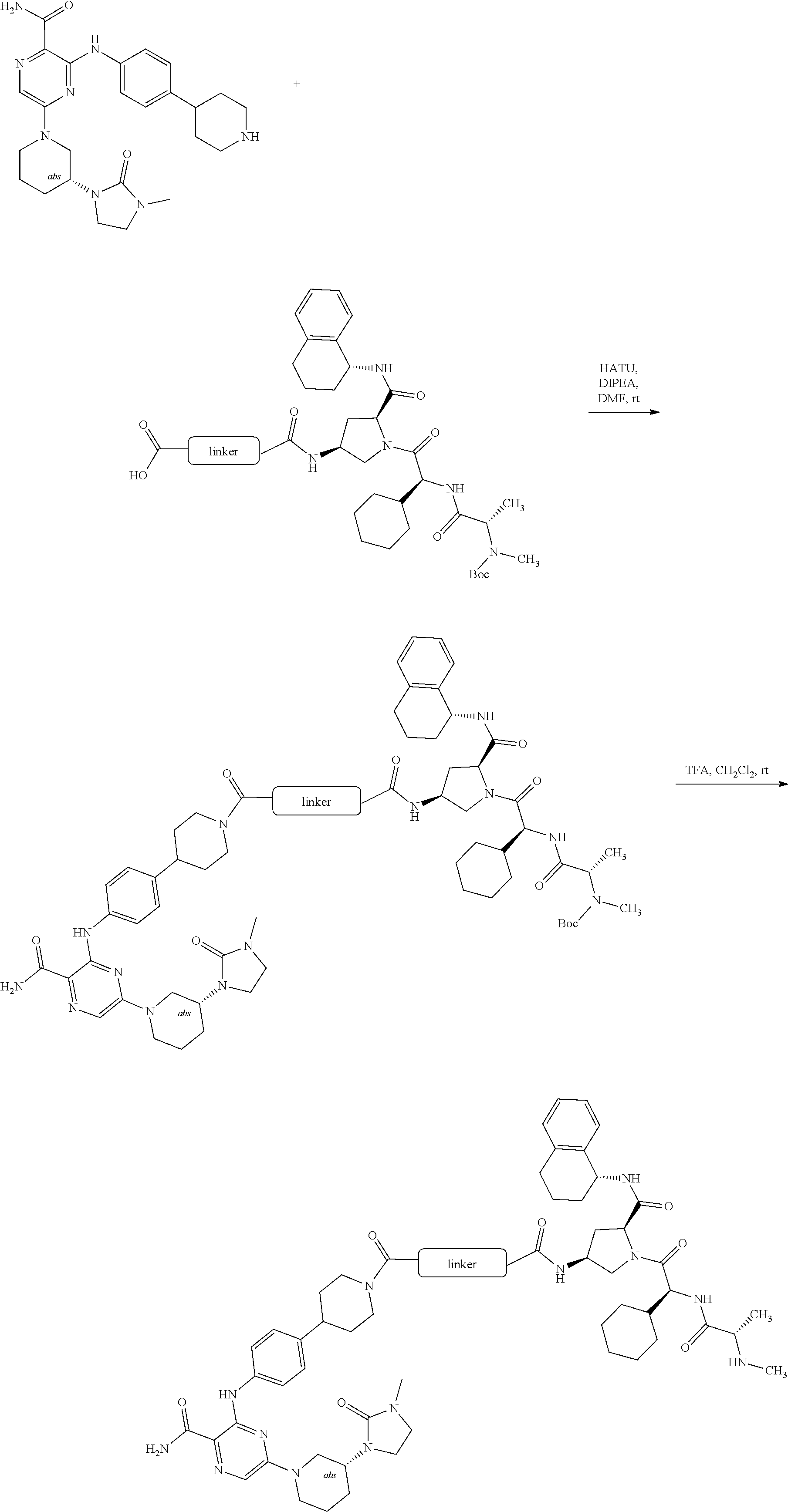

A mixture of amine (1 equiv), acid (1.1 equiv), HATU (1.2 equiv), DIPEA (3 equiv) and DMF (0.2 M) was allowed to stir at room temperature for 1 hour. EtOAc and H₂O were added. The organic layer was dried with MgSO₄, filtered, concentrated and carried to the next step.

TFA (20 equiv) and CH₂C₁₂ (0.1 M) were added and the mixture was allowed to stir at room temperature for 1 h. The volatiles were removed and the mixture was purified by HPLC (5-95% MeCN in H₂O with 0.1% TFA) to afford the desired product.

Physical Data for Example Compounds of Table 1.

The ¹H NMR spectra and mass spectrometry (LCMS) data were obtained for the example compounds reported in Table 1. These experimental data are provided in Table 2.

TABLE 2
Physical Data for the Example Compounds of Table 1.

| Compound | | Mass Spec |
|---|---|---|
| No. | ¹H NMR | (LCMS) |
| 1 | ¹H NMR (500 MHz, DMSO-d₆) δ 11.24 (d, J = 13.2 | LCMS: |
| | Hz, 1H), 11.09 (s, 1H), 7.78 (s, 1H), 7.74-7.63 (m, | C₃₅H₃₈N₁₀O₆ |
| | 0H), 7.68 (s, 1H), 7.60 (t, J = 7.8 Hz, 1H), 7.57- | requires: 694, |
| | 7.47 (m, 3H), 7.35 (s, 1H), 7.23 (d, J = 8.1 Hz, 2H), | found: m/z = 695 |
| | 7.16 (dd, J = 16.8, 8.4 Hz, 2H), 7.05 (d, J = 7.0 Hz, | [M + H]⁺. |
| | 1H), 6.58 (d, J = 6.3 Hz, 1H), 5.05 (dd, J = 12.8, 5.4 | |
| | Hz, 1H), 4.33 (s, 3H), 3.55 (d, J = 6.8 Hz, 2H), 3.25 | |
| | (s, 1H), 3.18 (s, 2H), 2.98 (d, J = 12.7 Hz, 1H), 2.92- | |
| | 2.83 (m, 3H), 2.76-2.67 (m, 4H), 2.61 (s, 1H), | |
| | 2.48 (s, 10H), 2.06-1.99 (m, 1H), 1.83 (d, J = 12.1 | |
| | Hz, 4H), 1.77 (s, 1H), 1.57 (d, J = 11.3 Hz, 2H), | |
| | 1.38 (d, J = 7.4 Hz, 1H), 1.31-1.23 (m, 2H). | |
| 2 | ¹H NMR (500 MHz, Acetonitrile-d₃) δ 11.25 (s, | LCMS |
| | 1H), 9.03 (s, 1H), 7.67-7.61 (m, 2H), 7.58 (dd, J = | C₄₃H₅₄N₁₀O₉ |
| | 8.8, 1.3 Hz, 2H), 7.49 (d, J = 1.0 Hz, 1H), 7.45 (s, | requires: 855, |
| | 2H), 7.42-7.32 (m, 2H), 6.94 (t, J = 5.9 Hz, 1H), | found: m/z = 856 |
| | 6.87 (dd, J = 16.7, 7.8 Hz, 2H), 6.31 (s, 1H), 5.82 (s, | [M + H]⁺. |
| | 1H), 5.54 (dd, J = 6.1, 2.3 Hz, 5H), 5.48 (dd, J = | |
| | 6.0, 2.1 Hz, 4H), 5.34 (dd, J = 6.0, 2.1 Hz, 2H), 4.84 | |
| | (ddd, J = 12.3, 5.4, 3.4 Hz, 1H), 4.30 (d, J = 12.9 | |
| | Hz, 1H), 4.20 (t, J = 7.1 Hz, 1H), 3.76 (dddd, J = | |
| | 15.7, 13.2, 8.1, 6.0 Hz, 25H), 3.65-3.41 (m, 16H), | |
| | 3.41 (q, J = 5.4 Hz, 2H), 3.32-3.13 (m, 6H), 2.96 | |
| | (t, J = 11.8 Hz, 1H), 2.88 (t, J = 12.9 Hz, 1H), 2.72- | |
| | 2.60 (m, 1H), 2.63-2.50 (m, 2H), 2.05-1.73 (m, | |
| | 19H), 1.81 (s, 13H), 1.76-1.52 (m, 24H), 1.19 (s, | |
| | 1H), 1.09 (d, J = 18.8 Hz, 1H). | |
| 3 | ¹H NMR (500 MHz, DMSO-d₆) δ 11.49 (s, 1H), | LCMS |
| | 11.02 (s, 1H), 8.24 (t, J = 5.6 Hz, 1H), 7.80-7.70 | C₄₂H₅₁N₁₁O₁₀ |
| | (m, 3H), 7.67 (s, 1H), 7.58 (dd, J = 9.1, 2.5 Hz, 2H), | requires: 870, |
| | 7.53-7.45 (m, 1H), 7.36 (d, J = 2.9 Hz, 1H), 7.08- | found: m/z = 871 |
| | 7.00 (m, 1H), 7.00-6.90 (m, 1H), 6.55-6.45 (m, | [M + H]⁺. |
| | 1H), 4.98 (dd, J = 12.7, 5.4 Hz, 1H), 4.26 (s, 2H), | |
| | 3.53 (dt, J = 10.7, 4.9 Hz, 5H), 3.19 (dd, J = 9.3, 7.3 | |
| | Hz, 3H), 3.03 (t, J = 11.8 Hz, 2H), 2.92 (t, J = 12.6 | |
| | Hz, 2H), 2.81 (ddd, J = 17.0, 13.8, 5.5 Hz, 1H), 2.64 | |
| | (s, 3H), 2.47 (s, 2H), 2.02-1.91 (m, 1H), 1.80- | |
| | 1.59 (m, 4H), 1.51 (d, J = 12.6 Hz, 1H). | |
| 4 | ¹H NMR (500 MHz, DMSO-d₆) δ 11.02 (s, 1H), | LCMS |
| | 10.98 (s, 1H), 9.77 (s, 1H), 8.01 (t, J = 5.7 Hz, 1H), | C₄₄H₅₄N₁₂O₈ |
| | 7.77-7.57 (m, 3H), 7.55 (s, 1H), 7.41 (d, J = 9.0 | requires: 879, |
| | Hz, 4H), 7.30-7.14 (m, 2H), 6.79 (d, J = 8.6 Hz, | found: m/z = 880 |
| | 2H), 5.09-4.88 (m, 1H), 4.41-4.02 (m, 5H), 3.88 | [M + H]⁺. |
| | (t, J = 6.3 Hz, 3H), 3.70-3.44 (m, 7H), 3.16 (ddt, | |
| | J = 22.1, 14.8, 8.7 Hz, 11H), 3.08-3.00 (m, 5H), | |
| | 3.00-2.77 (m, 5H), 2.64 (s, 4H), 2.15 (t, J = 7.0 | |
| | Hz, 3H), 1.96 (dt, J = 12.3, 5.7 Hz, 2H), 1.89-1.62 | |
| | (m, 9H), 1.54-1.40 (m, 1H). | |
| 5 | ¹H NMR (500 MHz, DMSO-d₆) δ 11.04 (s, 1H), | LCMS |
| | 10.96 (s, 1H), 7.78 (t, J = 5.6 Hz, 1H), 7.74-7.64 | C₄₄H₅₆N₁₂O₇ |
| | (m, 3H), 7.62 (dd, J = 6.9, 2.0 Hz, 1H), 7.55 (s, 1H), | requires: 865, |
| | 7.44-7.33 (m, 2H), 7.22 (s, 1H), 6.85-6.72 (m, | found: m/z = 866 |
| | 2H), 5.06 (dd, J = 12.8, 5.4 Hz, 1H), 4.23 (dd, J = | [M + H]⁺. |
| | 33.8, 12.8 Hz, 2H), 3.86 (t, J = 6.3 Hz, 3H), 3.34- | |
| | 3.07 (m, 10H), 3.05-2.75 (m, 6H), 2.63 (s, 3H), | |
| | 2.58-2.46 (m, 3H), 2.38 (ddd, J = 9.8, 7.7, 2.1 Hz, | |
| | 2H), 2.20 (ddd, J = 8.4, 6.5, 4.4 Hz, 2H), 1.98 (ddt, | |
| | J = 15.2, 9.4, 3.3 Hz, 2H), 1.87 (qd, J = 8.4, 2.7 Hz, | |
| | 2H), 1.80-1.66 (m, 7H), 1.47 (d, J = 11.3 Hz, 1H). | |
| 6 | ¹H NMR (500 MHz, DMSO-d₆) δ 10.96 (s, 1H), | LCMS |
| | 10.92 (s, 1H), 7.82 (t, J = 5.6 Hz, 1H), 7.65 (s, 1H), | C₄₀H₄₈N₁₀O₇ |
| | 7.55 (s, 1H), 7.50 (dd, J = 6.2, 2.4 Hz, 1H), 7.43- | requires: 781, |
| | 7.31 (m, 4H), 7.22 (s, 1H), 6.87-6.72 (m, 2H), 5.05 | found: m/z = 782 |
| | (dd, J = 13.3, 5.2 Hz, 1H), 4.46-4.11 (m, 4H), 3.86 | [M + H]⁺. |
| | (t, J = 6.3 Hz, 2H), 3.28-3.07 (m, 8H), 3.01-2.79 | |
| | (m, 3H), 2.62 (d, J = 1.8 Hz, 3H), 2.56 (dd, J = 9.6, | |
| | 6.7 Hz, 3H), 2.34 (dd, J = 13.2, 4.5 Hz, 3H), 2.05 (t, | |
| | J = 7.4 Hz, 2H), 1.94 (dd, J = 12.9, 6.1 Hz, 1H), | |
| | 1.82-1.65 (m, 7H), 1.54-1.40 (m, 1H). | |
| 7 | ¹H NMR (500 MHz, DMSO-d₆) δ 11.17 (s, 1H), | LCMS: |
| | 10.95 (s, 1H), 10.14 (s, 1H), 8.09 (d, J = 8.1 Hz, | C₄₅H₅₃N₁₃O₇ |
| | 1H), 7.69 (s, 1H), 7.60 (s, 1H), 7.47 (t, J = 8.7 Hz, | requires: 887, |
| | 3H), 7.35 (d, J = 7.5 Hz, 1H), 7.24 (t, J = 10.8 Hz, | found: m/z = 888 |
| | 3H), 5.07 (dd, J = 13.3, 5.1 Hz, 1H), 4.46-4.15 (m, | [M + H]⁺. |
| | 4H), 3.47-3.33 (m, 1H), 3.33-3.12 (m, 5H), 3.01- | |
| | 2.77 (m, 3H), 2.74 (t, J = 7.4 Hz, 2H), 2.63 (d, J = | |
| | 1.7 Hz, 3H), 2.60-2.51 (m, 1H), 2.33-2.15 (m, | |
| | 1H), 2.05-1.40 (m, 9H), 1.15 (s, 3H). | |
| 8 | ¹H NMR (500 MHz, DMSO-d₆) δ 11.49 (s, 1H), | LCMS |
| | 11.01 (s, 1H), 8.26 (t, J = 5.7 Hz, 1H), 7.78-7.70 | C₃₈H₄₃N₁₁O₇ |
| | (m, 3H), 7.67 (s, 1H), 7.58 (d, J = 8.7 Hz, 2H), 7.50 | requires: 766, |
| | (dd, J = 8.6, 7.1 Hz, 1H), 7.36 (s, 1H), 7.05 (d, J = | found: m/z = 767 |
| | 8.6 Hz, 1H), 6.94 (d, J = 7.0 Hz, 1H), 6.51 (s, 1H), | [M + H]⁺. |
| | 4.98 (dd, J = 12.8, 5.5 Hz, 1H), 4.27 (s, 2H), 3.31- | |
| | 3.14 (m, 6H), 3.03 (t, J = 11.8 Hz, 1H), 2.92 (t, J = | |
| | 12.1 Hz, 1H), 2.81 (ddd, J = 16.8, 13.8, 5.4 Hz, 1H), | |
| | 2.63 (s, 3H), 1.95 (ddt, J = 12.9, 5.4, 3.3 Hz, 1H), | |
| | 1.80-1.66 (m, 3H), 1.58-1.48 (m, 6H). | |
| 9 | ¹H NMR (500 MHz, DMSO-d₆) δ 11.50 (s, 1H), | LCMS |
| | 10.91 (s, 1H), 8.21 (t, J = 5.6 Hz, 1H), 7.77 (s, 1H), | C₃₈H₄₄N₁₀O₇ |
| | 7.74-7.66 (m, 3H), 7.61-7.55 (m, 2H), 7.50 (d, J = | requires: 753, |
| | 7.6 Hz, 1H), 7.44 (d, J = 7.6 Hz, 1H), 7.38-7.31 | found: m/z = 754 |
| | (m, 2H), 5.05 (dd, J = 13.3, 5.1 Hz, 1H), 4.42 (dd, | [M + H]⁺. |
| | J = 17.1, 1.8 Hz, 1H), 4.32-4.24 (m, 3H), 3.68- | |
| | 3.54 (m, 1H), 3.44 (t, J = 6.1 Hz, 1H), 3.36-3.14 | |
| | (m, 4H), 3.04 (t, J = 11.7 Hz, 1H), 2.93 (t, J = 12.1 | |
| | Hz, 1H), 2.90-2.77 (m, 3H), 2.63 (s, 3H), 2.52 (d, | |
| | J = 18.9 Hz, 1H), 2.38-2.29 (m, 1H), 1.92 (ddd, J = | |
| | 12.2, 6.2, 3.7 Hz, 1H), 1.73 (dtd, J = 18.4, 11.8, | |
| | 10.7, 5.3 Hz, 3H), 1.51 (d, J = 11.6 Hz, 1H). | |
| 10 | ¹H NMR (500 MHz, DMSO-d₆) δ 11.30-11.13 (m, | LCMS: |
| | 1H), 11.04 (s, 1H), 7.72 (dd, J = 7.7, 2.2 Hz, 1H), | C₄₅H₅₄N₁₀O₈ |
| | 7.68 (d, J = 6.6 Hz, 1H), 7.59 (d, J = 3.6 Hz, 2H), | requires: 862, |
| | 7.50-7.43 (m, 2H), 7.31-7.15 (m, 3H), 5.11- | found: m/z = 863 |
| | 5.00 (m, 1H), 4.26 (dd, J = 50.5, 13.4 Hz, 2H), 3.63- | [M + H]⁺. |
| | 3.08 (m, 9H), 3.00-2.76 (m, 2H), 2.72 (t, J = 7.6 | |
| | Hz, 2H), 2.63 (d, J = 1.2 Hz, 2H), 2.60-2.45 (m, | |
| | 3H), 2.05-1.39 (m, 11H), 1.14 (s, 3H). | |
| 11 | ¹H NMR (500 MHz, DMSO-d₆) δ 11.31-11.16 (m, | LCMS: |
| | 1H), 11.02 (s, 1H), 9.61 (s, 1H), 7.70 (d, J = 8.3 Hz, | C₄₇H₅₈N₁₂O₇ |
| | 2H), 7.61 (d, J = 7.0 Hz, 1H), 7.53-7.47 (m, 2H), | requires: 902, |
| | 7.42 (d, J = 2.2 Hz, 1H), 7.33-7.19 (m, 4H), 5.03 | found: m/z = 903 |
| | (dd, J = 12.8, 5.5 Hz, 1H), 4.41-4.06 (m, 4H), 3.60- | [M + H]⁺. |
| | 3.33 (m, 4H), 3.33-3.02 (m, 11H), 3.01-2.73 | |
| | (m, 3H), 2.64 (d, J = 2.5 Hz, 3H), 2.62-2.46 (m, | |
| | 2H), 2.02-1.41 (m, 12H), 1.16 (d, J = 1.8 Hz, 3H). | |
| 12 | ¹H NMR (500 MHz, DMSO-d₆) δ 10.98 (d, J = 6.3 | LCMS: |
| | Hz, 2H), 7.66 (s, 1H), 7.61-7.54 (m, 2H), 7.45 (d, | C₄₅H₅₇N₁₃O₆ |
| | J = 6.9 Hz, 2H), 7.43-7.38 (m, 2H), 7.22 (s, 1H), | requires: 875, |
| | 6.89-6.84 (m, 2H), 5.10 (dd, J = 13.3, 5.2 Hz, 1H), | found: m/z = 876 |
| | 4.45 (d, J = 17.0 Hz, 1H), 4.36-4.14 (m, 3H), 3.61- | [M + H]⁺. |
| | 3.44 (m, 5H), 3.34-3.14 (m, 4H), 3.11-2.80 (m, | |
| | 9H), 2.64 (s, 3H), 2.61-2.53 (m, 1H), 2.33-2.24 | |
| | (m, 1H), 2.04-1.91 (m, 1H), 1.80-1.64 (m, 4H), | |
| | 1.48 (d, J = 12.8 Hz, 1H). | |
| 13 | ¹H NMR (500 MHz, DMSO-d₆) δ 11.24 (dd, J = | LCMS: |
| | 42.3, 5.8 Hz, 1H), 10.96 (s, 1H), 7.71 (s, 1H), 7.62 | C₄₇H₆₀N₁₂O₆ |
| | (d, J = 7.0 Hz, 1H), 7.57 (d, J = 6.5 Hz, 1H), 7.53- | requires: 888, |
| | 7.49 (m, 2H), 7.44 (d, J = 7.9 Hz, 2H), 7.31-7.20 | found: m/z = 889 |
| | (m, 3H), 5.10 (dd, J = 13.3, 5.1 Hz, 1H), 4.44 (d, J = | [M + H]⁺. |
| | 17.1 Hz, 1H), 4.37-4.18 (m, 2H), 3.34-3.10 (m, | |
| | 4H), 3.04-2.78 (m, 4H), 2.67-2.53 (m, 4H), 2.29 | |
| | (p, J = 1.9 Hz, 1H), 1.94 (d, J = 33.6 Hz, 3H), 1.82- | |
| | 1.40 (m, 7H), 1.17 (d, J = 1.9 Hz, 3H). | |
| 14 | ¹H NMR (500 MHz, DMSO-d₆) δ 11.22 (d, J = 56.8 | LCMS: |
| | Hz, 1H), 10.94 (s, 1H), 9.73 (s, 1H), 7.77-7.66 (m, | C₄₅H₅₅N₁₁O₈ |
| | 2H), 7.60 (d, J = 5.3 Hz, 1H), 7.44 (ddd, J = 20.1, | requires: 877, |
| | 15.6, 8.0 Hz, 4H), 7.25 (d, J = 13.9 Hz, 1H), 7.23- | found: m/z = 878 |
| | 7.16 (m, 2H), 5.07 (dd, J = 13.3, 5.1 Hz, 1H), 4.38- | [M + H]⁺. |
| | 4.15 (m, 5H), 3.63 (t, J = 6.3 Hz, 2H), 3.60-3.48 | |
| | (m, 2H), 3.39 (d, J = 8.8 Hz, 1H), 3.35-3.11 (m, | |
| | 5H), 3.01-2.77 (m, 3H), 2.63 (s, 3H), 2.60-2.47 | |
| | (m, 4H), 2.34-2.20 (m, 1H), 2.01-1.85 (m, 1H), | |
| | 1.85-1.63 (m, 3H), 1.60-1.39 (m, 0H), 1.10 (d, | |
| | J = 3.3 Hz, 3H). | |
| 15 | ¹H NMR (500 MHz, Methanol-d₄) δ 7.71 (dd, J = | LCMS: |
| | 7.2, 1.5 Hz, 1H), 7.60 (s, 1H), 7.58-7.51 (m, 4H), | C₄₀H₄₉N₁₁O₅ |
| | 7.01-6.95 (m, 2H), 5.20 (dd, J = 13.3, 5.2 Hz, 1H), | requires: 763, |
| | 4.59-4.45 (m, 2H), 4.36 (dd, J = 33.5, 12.9 Hz, | found: m/z = 764 |
| | 2H), 3.83-3.60 (m, 3H), 3.52-3.32 (m, 6H), 3.12 | [M + H]⁺. |
| | (dd, J = 12.8, 10.6 Hz, 1H), 3.05-2.88 (m, 4H), | |
| | 2.87-2.81 (m, 2H), 2.78 (d, J = 8.1 Hz, 4H), 2.65 | |
| | (s, 1H), 2.51 (dd, J = 13.2, 4.6 Hz, 1H), 2.24-2.11 | |
| | (m, 4H), 2.03-1.79 (m, 4H). | |
| 16 | ¹H NMR (500 MHz, Methanol-d₄) δ 7.73-7.51 (m, | LCMS: |
| | 4H), 7.49-7.45 (m, 1H), 7.35-7.27 (m, 2H), 5.18 | C₄₂H₅₂N₁₀O₅ |
| | (ddd, J = 19.2, 13.4, 5.2 Hz, 1H), 4.43 (ddd, J = | requires: 776, |
| | 53.8, 36.6, 16.1 Hz, 4H), 3.83-3.70 (m, 1H), 3.60- | found: m/z = 777 |
| | 3.32 (m, 3H), 3.16-2.69 (m, 9H), 2.67-2.38 (m, | [M + H]⁺. |
| | 2H), 2.26-1.55 (m, 9H), 1.39 (s, 1H), 1.25 (s, 2H). | |
| 17 | ¹H NMR (500 MHz, DMSO-d₆) δ 10.91 (d, J = 6.2 | LCMS: |
| | Hz, 2H), 7.64 (d, J = 2.8 Hz, 1H), 7.53 (s, 1H), 7.50 | C₄₁H₅₁N₁₁O₅ |
| | (dd, J = 5.8, 2.8 Hz, 1H), 7.43-7.37 (m, 2H), 7.37- | requires: 777, |
| | 7.32 (m, 2H), 7.20 (d, J = 2.9 Hz, 1H), 6.82-6.76 | found: m/z = 778 |
| | (m, 2H), 5.06 (dd, J = 13.3, 5.1 Hz, 1H), 4.40 (d, J = | [M + H]⁺. |
| | 17.1 Hz, 1H), 4.33-4.14 (m, 3H), 3.58-3.49 (m, | |
| | 3H), 3.04-2.79 (m, 8H), 2.65-2.47 (m, 7H), 2.33- | |
| | 2.24 (m, 2H), 2.15 (t, J = 7.4 Hz, 1H), 1.94 (dd, | |
| | J = 11.4, 6.0 Hz, 1H), 1.80-1.65 (m, 6H), 1.63- | |
| | 1.36 (m, 5H), 1.17 (s, 3H). | |
| 18 | ¹H NMR (500 MHz, DMSO-d₆) δ 11.20 (d, J = 16.7 | LCMS: |
| | Hz, 1H), 11.05 (d, J = 6.4 Hz, 2H), 9.15 (d, J = 83.4 | C₄₄H₅₄N₁₀O₇ |
| | Hz, 1H), 7.77-7.57 (m, 6H), 7.54-7.38 (m, 3H), | requires: 834, |
| | 7.31-7.14 (m, 4H), 5.05 (dt, J = 12.7, 5.2 Hz, 2H), | found: m/z = 835 |
| | 4.37-4.01 (m, 3H), 3.09-2.72 (m, 8H), 2.62 (s, | [M + H]⁺. |
| | 4H), 2.07-1.63 (m, 12H), 1.28-1.04 (m, 5H). | |
| 19 | ¹H NMR (500 MHz, DMSO-d₆) δ 10.99 (d, J = 30.5 | LCMS: |
| | Hz, 1H), 7.65 (s, 1H), 7.58-7.47 (m, 2H), 7.37 (dd, | C₄₂H₅₀N₁₂O₈ |
| | J = 8.8, 4.4 Hz, 2H), 7.22 (s, 1H), 7.07 (d, J = 8.7 | requires: 850, |
| | Hz, 1H), 6.97 (d, J = 7.1 Hz, 1H), 6.82 (d, J = 8.8 | found: m/z = 851 |
| | Hz, 2H), 6.52 (s, 1H), 4.98 (dd, J = 13.0, 5.4 Hz, | [M + H]⁺. |
| | 1H), 4.24 (dd, J = 28.7, 12.9 Hz, 2H), 3.68-3.45 | |
| | (m, 23H), 3.40 (d, J = 5.1 Hz, 2H), 3.31-3.13 (m, | |
| | 3H), 3.05-2.71 (m, 7H), 2.63 (d, J = 1.6 Hz, 2H), | |
| | 2.60-2.53 (m, 2H), 1.93 (dd, J = 12.8, 5.9 Hz, 1H), | |
| | 1.79-1.62 (m, 2H), 1.48 (d, J = 12.9 Hz, 1H). | |
| 20 | ¹H NMR (500 MHz, DMSO-d₆) δ 11.20 (d, J = 6.4 | LCMS: |
| | Hz, 1H), 10.95 (s, 1H), 7.71 (s, 1H), 7.61 (s, 1H), | C₄₈H₆₂N₁₂O₆ |
| | 7.52 (ddd, J = 21.3, 6.8, 2.8 Hz, 3H), 7.43 (d, J = | requires: 902, |
| | 3.6 Hz, 2H), 7.30-7.21 (m, 3H), 5.09 (dd, J = 13.3, | found: m/z = 903 |
| | 5.2 Hz, 1H), 4.41 (d, J = 17.0 Hz, 1H), 4.24 (dd, J = | [M + H]⁺. |
| | 16.1, 10.8 Hz, 2H), 3.32-3.11 (m, 4H), 3.08-2.79 | |
| | (m, 4H), 2.67-2.51 (m, 8H), 2.34-2.24 (m, 1H), | |
| | 2.04-1.82 (m, 5H), 1.81-1.41 (m, 8H), 1.16 (d, | |
| | J = 2.0 Hz, 3H). | |
| 21 | ¹H NMR (500 MHz, DMSO-d₆) δ 11.14 (s, 1H), | LCMS: |
| | 10.91 (s, 1H), 7.68 (d, J = 2.8 Hz, 1H), 7.60 (d, J = | C₄₃H₅₄N₁₀O₅ |
| | 4.8 Hz, 1H), 7.52-7.43 (m, 3H), 7.38 (d, J = 4.4 | requires: 790, |
| | Hz, 2H), 7.26 (d, J = 2.8 Hz, 1H), 7.19 (d, J = 8.4 | found: m/z = 791 |
| | Hz, 2H), 5.06 (dd, J = 13.3, 5.1 Hz, 1H), 4.38 (d, J = | [M + H]⁺. |
| | 17.1 Hz, 1H), 4.22 (dd, J = 15.5, 10.4 Hz, 3H), 3.60- | |
| | 3.47 (m, 1H), 3.21-3.11 (m, 2H), 3.05-2.73 (m, | |
| | 3H), 2.65-2.48 (m, 6H), 2.37-2.10 (m, 4H), 1.98- | |
| | 1.83 (m, 3H), 1.82-1.30 (m, 11H), 1.07 (s, 3H). | |
| 22 | ¹H NMR (500 MHz, DMSO-d₆) δ 11.20 (d, J = 6.5 | LCMS: |
| | Hz, 1H), 11.06 (s, 1H), 7.77-7.65 (m, 4H), 7.61 (s, | C₄₈H₆₀N₁₂O₇ |
| | 1H), 7.50 (dd, J = 8.8, 2.4 Hz, 2H), 7.30-7.21 (m, | requires: 916, |
| | 3H), 5.06 (dd, J = 12.8, 5.5 Hz, 1H), 4.27 (dd, J = | found: m/z = 917 |
| | 46.1, 13.1 Hz, 2H), 3.59-3.15 (m, 8H), 3.07-2.77 | [M + H]⁺. |
| | (m, 7H), 2.68-2.47 (m, 5H), 2.07-1.83 (m, 6H), | |
| | 1.81-1.41 (m, 6H), 1.16 (d, J = 1.9 Hz, 3H). | |
| 23 | ¹H NMR (500 MHz, DMSO-d₆) δ 11.13 (d, J = 4.1 | LCMS: |
| | Hz, 1H), 10.92 (s, 1H), 7.69 (s, 1H), 7.59 (s, 1H), | C₄₃H₅₂N₁₀O₆ |
| | 7.50 (dd, J = 6.2, 2.4 Hz, 1H), 7.46-7.37 (m, 4H), | requires: 804, |
| | 7.26 (s, 1H), 7.07 (d, J = 8.4 Hz, 2H), 5.07 (dd, J = | found: m/z = 805 |
| | 13.3, 5.2 Hz, 1H), 4.43 (dd, J = 25.5, 15.3 Hz, 2H), | [M + H]⁺. |
| | 4.34-4.18 (m, 2H), 3.89 (d, J = 13.4 Hz, 1H), 3.31- | |
| | 3.11 (m, 4H), 3.06-2.79 (m, 4H), 2.70-2.46 (m, | |
| | 8H), 2.40-2.21 (m, 3H), 2.00-1.89 (m, 1H), 1.83- | |
| | 1.18 (m, 12H). | |
| 24 | ¹H NMR (500 MHz, DMSO-d₆) δ 11.21 (d, J = 4.4 | LCMS: |
| | Hz, 1H), 11.09 (s, 1H), 7.76 (s, 1H), 7.66 (s, 1H), | C₄₇H₅₉N₁₁O₁₀ |
| | 7.58 (dd, J = 8.6, 7.1 Hz, 1H), 7.50 (d, J = 8.2 Hz, | requires: 937, |
| | 2H), 7.33 (s, 1H), 7.19-7.11 (m, 3H), 7.04 (d, J = | found: m/z = 938 |
| | 7.0 Hz, 1H), 6.60 (s, 1H), 5.06 (dd, J = 12.7, 5.4 Hz, | [M + H]⁺. |
| | 1H), 4.53 (d, J = 12.8 Hz, 1H), 4.32 (dd, J = 32.0, | |
| | 12.9 Hz, 2H), 3.98 (d, J = 13.4 Hz, 1H), 3.68-3.48 | |
| | (m, 11H), 3.38-3.16 (m, 4H), 3.10-2.82 (m, 4H), | |
| | 2.70 (s, 3H), 2.63-2.53 (m, 3H), 2.08-1.97 (m, | |
| | 1H), 1.86-1.68 (m, 5H), 1.63-1.28 (m, 3H). | |
| 25 | ¹H NMR (500 MHz, DMSO-d₆) δ 11.13 (d, J = 2.7 | LCMS: |
| | Hz, 1H), 11.02 (s, 1H), 7.68 (s, 1H), 7.59 (s, 1H), | C₄₃H₅₁N₁₁O₈ |
| | 7.51 (dd, J = 8.5, 7.1 Hz, 1H), 7.44-7.38 (m, 2H), | requires: 849, |
| | 7.26 (s, 1H), 7.07 (dd, J = 8.6, 6.7 Hz, 3H), 6.97 (d, | found: m/z = 850 |
| | J = 7.0 Hz, 1H), 6.53 (s, 1H), 4.97 (dd, J = 12.9, 5.4 | [M + H]⁺. |
| | Hz, 1H), 4.45 (d, J = 12.9 Hz, 1H), 4.34-4.14 (m, | |
| | 2H), 3.91 (d, J = 13.4 Hz, 1H), 3.63 (t, J = 6.6 Hz, | |
| | 2H), 3.58-3.44 (m, 8H), 3.44-3.34 (m, 3H), 3.29- | |
| | 3.09 (m, 4H), 3.04-2.75 (m, 4H), 2.62 (s, 4H), | |
| | 1.92 (qd, J = 6.9, 4.2, 3.5 Hz, 1H), 1.78-1.20 (m, | |
| | 7H). | |
| 26 | ¹H NMR (500 MHz, DMSO-d₆) δ 11.17 (d, J = 5.1 | LCMS: |
| | Hz, 1H), 10.92 (s, 1H), 7.69 (s, 1H), 7.60 (s, 1H), | C₄₄H₅₄N₁₀O₆ |
| | 7.52-7.45 (m, 3H), 7.40-7.36 (m, 2H), 7.30- | requires: 818, |
| | 7.18 (m, 3H), 5.06 (dd, J = 13.3, 5.1 Hz, 1H), 4.45- | found: m/z = 819 |
| | 4.16 (m, 4H), 3.32-3.11 (m, 5H), 3.01-2.80 (m, | [M + H]⁺. |
| | 3H), 2.68-2.50 (m, 6H), 2.38-2.20 (m, 3H), 1.98- | |
| | 1.63 (m, 4H), 1.61-1.40 (m, 8H), 1.13 (s, 3H). | |
| 27 | ¹H NMR (500 MHz, DMSO-d₆) δ 11.17 (d, J = 4.1 | LCMS: |
| | Hz, 1H), 11.02 (s, 1H), 7.69 (s, 1H), 7.59 (s, 1H), | C₄₈H₆₁N₁₁O₁₀ |
| | 7.53-7.45 (m, 3H), 7.31-7.18 (m, 3H), 7.05 (d, J = | requires: 951, |
| | 8.6 Hz, 1H), 6.96 (d, J = 7.0 Hz, 1H), 6.52 (s, 1H), | found: m/z = 952 |
| | 4.98 (dd, J = 12.8, 5.4 Hz, 1H), 4.27 (dd, J = 52.2, | [M + H]⁺. |
| | 13.5 Hz, 2H), 3.32-3.11 (m, 4H), 2.99-2.75 (m, | |
| | 3H), 2.63 (d, J = 1.2 Hz, 2H), 2.60-2.45 (m, 4H), | |
| | 2.02-1.43 (m, 10H), 1.13 (s, 3H). | |
| 28 | ¹H NMR (500 MHz, DMSO-d₆) δ 11.16 (d, J = 2.7 | LCMS: |
| | Hz, 1H), 11.02 (s, 1H), 7.69 (s, 1H), 7.60 (s, 1H), | C₄₄H₅₃N₁₁O₈ |
| | 7.51-7.43 (m, 4H), 7.26 (s, 1H), 7.22-7.18 (m, | requires: 863, |
| | 2H), 7.05 (d, J = 8.6 Hz, 1H), 6.96 (d, J = 7.0 Hz, | found: m/z = 864 |
| | 1H), 6.50 (t, J = 5.8 Hz, 1H), 4.98 (dd, J = 12.7, 5.4 | [M + H]⁺. |
| | Hz, 1H), 4.27 (dd, J = 49.9, 13.2 Hz, 2H), 3.29- | |
| | 3.13 (m, 4H), 2.99-2.75 (m, 3H), 2.63 (s, 3H), 2.59- | |
| | 2.47 (m, 3H), 2.00-1.43 (m, 7H), 1.12 (s, 3H). | |
| 29 | ¹H NMR (500 MHz, DMSO-d₆) δ 11.08 (s, 1H), | LCMS |
| | 7.77 (s, 1H), 7.70-7.62 (m, 4H), 7.57 (s, 1H), 7.39 | C₄₂H₄₉N₁₁O₆ |
| | (s, 2H), 7.33 (d, J = 16.2 Hz, 3H), 5.07 (dd, J = 12.8, | requires: 804, |
| | 5.4 Hz, 2H), 4.36 (d, J = 12.3 Hz, 2H), 4.30 (d, J = | found: m/z = 805 |
| | 13.3 Hz, 2H), 3.62 (s, 4H), 3.05 (s, 2H), 3.01-2.85 | [M + H]⁺. |
| | (m, 6H), 2.67 (s, 6H), 2.41 (s, 3H), 2.05-1.99 (m, | |
| | 2H), 1.83 (d, J = 11.8 Hz, 5H), 1.77 (d, J = 9.7 Hz, | |
| | 3H), 1.69 (s, 5H), 1.58 (s, 3H), 1.25 (s, 1H). | |
| 30 | ¹H NMR (500 MHz, DMSO-d₆) δ 11.44 (s, 1H), | LCMS: |
| | 11.09 (d, J = 10.0 Hz, 1H), 10.81 (s, 1H), 7.83 (s, | C₃₉H₄₃N₁₁O₆ |
| | 1H), 7.79-7.63 (m, 3H), 7.41 (s, 2H), 7.16 (d, J = | expected: 761.8. |
| | 8.2 Hz, 1H), 6.93 (s, 1H), 6.84-6.73 (m, 1H), 5.08 | Found [M + H}: 762.7 |
| | (ddd, J = 15.5, 10.1, 5.4 Hz, 1H), 4.65-4.02 (m, | |
| | 10H), 3.28 (dd, J = 9.9, 7.2 Hz, 3H), 3.10 (d, J = | |
| | 15.2 Hz, 5H), 3.04-2.74 (m, 4H), 2.71 (s, 4H), | |
| | 2.60 (d, J = 19.6 Hz, 2H), 2.04 (dq, J = 11.9, 6.0, | |
| | 4.8 Hz, 1H), 1.81 (td, J = 18.8, 16.1, 10.1 Hz, 3H), | |
| | 1.59 (d, J = 11.5 Hz, 1H). | |
| 31 | ¹H NMR (500 MHz, DMSO-d₆) δ 11.44 (d, J = 9.7 | LCMS |
| | Hz, 1H), 11.09 (s, 1H), 9.58 (s, 1H), 7.83 (s, 1H), | C₄₂H₄₉N₁₁O₆ |
| | 7.80-7.62 (m, 3H), 7.51-7.25 (m, 4H), 7.16 (d, J = | requires: 804, |
| | 8.5 Hz, 1H), 5.08 (dd, J = 12.8, 5.4 Hz, 1H), 4.58 | found: m/z = 805 |
| | (d, J = 15.3 Hz, 1H), 4.43-4.22 (m, 3H), 4.13 (d, J = | [M + H]⁺. |
| | 13.2 Hz, 2H), 3.10-2.85 (m, 8H), 2.70 (d, J = 2.7 | |
| | Hz, 4H), 2.15-1.68 (m, 7H), 1.59 (d, J = 12.4 Hz, | |
| | 1H), 1.31 (qd, J = 17.9, 16.1, 8.7 Hz, 2H). | |
| 32 | ¹H NMR (500 MHz, DMSO-d₆) δ 11.20 (s, 1H), | LCMS: |
| | 11.07 (s, 1H), 7.76 (s, 1H), 7.67 (t, J = 4.3 Hz, 2H), | C₄₄H₅₃N₁₁O₆ |
| | 7.52 (d, J = 8.3 Hz, 2H), 7.32 (d, J = 21.4 Hz, 2H), | requires: 831, |
| | 7.21 (t, J = 8.3 Hz, 3H), 5.06 (dd, J = 12.9, 5.4 Hz, | found: m/z = 832 |
| | 1H), 4.44-4.25 (m, 2H), 3.89 (s, 3H), 3.62 (d, J = | [M + H]⁺. |
| | 11.2 Hz, 1H), 3.10-2.81 (m, 6H), 2.73 (s, 3H), | |
| | 2.69-2.57 (m, 2H), 2.41-2.26 (m, 1H), 2.22- | |
| | 1.45 (m, 19H), 1.26 (d, J = 17.1 Hz, 4H). | |
| 33 | ¹H NMR (500 MHz, DMSO-d₆) δ 11.01 (s, 1H), | LCMS: |
| | 10.91 (s, 1H), 7.70 (s, 1H), 7.61-7.54 (m, 2H), | C₄₁H₄₉N₁₁O₅ |
| | 7.50-7.44 (m, 2H), 7.40-7.34 (m, 2H), 7.26 (d, J = | requires: 775, |
| | 2.8 Hz, 1H), 6.38 (d, J = 8.7 Hz, 2H), 5.15 (dd, J = | found: m/z = 776 |
| | 13.3, 5.2 Hz, 1H), 4.48 (d, J = 17.1 Hz, 1H), 4.41- | [M + H]⁺. |
| | 4.19 (m, 3H), 3.81 (s, 4H), 3.60 (d, J = 5.6 Hz, | |
| | 1H), 3.24 (s, 4H), 2.96 (dt, J = 36.5, 13.5 Hz, 4H), | |
| | 2.72 (s, 3H), 2.65 (dd, J = 9.7, 5.5 Hz, 3H), 2.39- | |
| | 2.30 (m, 2H), 2.07-1.99 (m, 1H), 1.87-1.70 (m, | |
| | 3H), 1.62 (t, J = 7.5 Hz, 2H), 1.24 (d, J = 9.4 Hz, | |
| | 1H). | |
| 34 | ¹H NMR (500 MHz, DMSO-d₆) δ 11.08 (s, 1H), | LCMS: |
| | 10.94 (s, 1H), 7.73-7.65 (m, 2H), 7.60 (s, 1H), | C₃₈H₄₁N₁₁O₆ |
| | 7.41 (d, J = 8.7 Hz, 2H), 7.27 (d, J = 3.0 Hz, 1H), | requires: 747, |
| | 6.84 (d, J = 2.0 Hz, 1H), 6.70 (dd, J = 8.4, 2.1 Hz, | found: m/z = 748 |
| | 1H), 6.44 (d, J = 8.6 Hz, 2H), 5.07 (dd, J = 12.8, 5.4 | [M + H]⁺. |
| | Hz, 1H), 4.37 (d, J = 12.4 Hz, 1H), 4.26 (s, 4H), | |
| | 3.99 (s, 4H), 3.05-2.83 (m, 3H), 2.73 (s, 3H), 2.67- | |
| | 2.55 (m, 2H), 2.08-1.97 (m, 1H), 1.91-1.69 (m, | |
| | 3H), 1.56 (d, J = 12.9 Hz, 1H). | |
| 35 | ¹H NMR (500 MHz, DMSO-d₆) δ 11.27 (s, 1H), | LCMS: |
| | 11.07 (s, 1H), 7.77 (d, J = 2.8 Hz, 1H), 7.70-7.62 | C₄₁H₄₇N₁₁O₆ |
| | (m, 2H), 7.55 (d, J = 8.5 Hz, 2H), 7.35 (d, J = 2.8 | requires 789, |
| | Hz, 1H), 7.27 (d, J = 8.5 Hz, 2H), 6.91 (d, J = 2.2 | found: m/z = 790 |
| | Hz, 1H), 6.83 (dd, J = 8.6, 2.2 Hz, 1H), 5.06 (dd, J = | [M + H]⁺. |
| | 12.9, 5.4 Hz, 1H), 4.39 (d, J = 12.4 Hz, 1H), 4.30 (d, | |
| | J = 13.3 Hz, 1H), 3.76-3.44 (m, 6H), 3.44-3.19 | |
| | (m, 5H), 3.18-2.83 (m, 6H), 2.73 (s, 3H), 2.66- | |
| | 2.30 (m, 5H), 2.20-1.96 (m, 2H), 1.91-1.68 (m, | |
| | 4H), 1.61-1.54 (m, 1H). | |
| 36 | ¹H NMR (500 MHz, DMSO-d₆) δ 11.28 (s, 1H), | LCMS: |
| | 11.01 (s, 1H), 7.78 (d, J = 2.8 Hz, 1H), 7.68 (s, 1H), | C₄₀H₄₈N₁₀O₅ |
| | 7.62-7.53 (m, 3H), 7.50-7.46 (m, 2H), 7.36 (d, J = | requires 748, |
| | 2.9 Hz, 1H), 7.27 (d, J = 8.1 Hz, 2H), 5.15 (dd, J = | found: m/z = 749 |
| | 13.3, 5.1 Hz, 1H), 4.48 (d, J = 17.1 Hz, 1H), 4.34 | [M + H]⁺. |
| | (dd, J = 24.1, 12.5 Hz, 3H), 3.69-3.54 (m, 1H), | |
| | 3.42-3.19 (m, 4H), 3.09-2.88 (m, 3H), 2.76- | |
| | 2.23 (m, 14H), 2.06-1.99 (m, 1H), 1.89-1.72 (m, | |
| | 3H), 1.69-1.54 (m, 3H), 1.48-1.32 (m, 2H). | |
| 37 | ¹H NMR (500 MHz, DMSO-d₆) δ 11.26 (s, 1H), | LCMS: |
| | 11.08 (s, 1H), 7.77 (d, J = 2.9 Hz, 1H), 7.70-7.64 | C₄₁H₄₇N₁₁O₆ |
| | (m, 2H), 7.55 (d, J = 8.3 Hz, 2H), 7.37-7.29 (m, | requires 789, |
| | 2H), 7.30-7.23 (m, 3H), 5.08 (dd, J = 12.8, 5.4 Hz, | found: m/z = 790 |
| | 1H), 4.40 (d, J = 12.6 Hz, 1H), 4.30 (d, J = 13.6 Hz, | [M + H]⁺. |
| | 1H), 3.88 (dt, J = 13.5, 4.5 Hz, 2H), 3.67-3.49 (m, | |
| | 4H), 3.41-3.22 (m, 2H), 3.16 (ddd, J = 13.0, 9.8, | |
| | 3.1 Hz, 2H), 3.10-2.83 (m, 5H), 2.72 (s, 3H), 2.65- | |
| | 2.53 (m, 2H), 2.42-2.33 (m, 1H), 2.08-1.96 (m, | |
| | 1H), 1.86-1.71 (m, 6H), 1.60-1.50 (m, 1H), 1.33- | |
| | 1.22 (m, 3H). | |
| 38 | ¹H NMR (500 MHz, DMSO-d₆) δ 11.06 (s, 1H), | LCMS: |
| | 10.99 (s, 1H), 7.71 (d, J = 3.0 Hz, 1H), 7.65-7.58 | C₄₃H₅₀N₁₂O₆ |
| | (m, 2H), 7.42 (d, J = 8.8 Hz, 2H), 7.27 (d, J = 2.9 | requires: 830, |
| | Hz, 1H), 6.94-6.86 (m, 2H), 6.89-6.79 (m, 2H), | found: m/z = 831 |
| | 5.05 (dd, J = 12.9, 5.4 Hz, 1H), 4.35 (d, J = 12.1 Hz, | [M + H]⁺. |
| | 1H), 4.27 (d, J = 13.4 Hz, 1H), 3.68 (d, J = 10.3 Hz, | |
| | 2H), 3.60 (ddt, J = 10.9, 7.8, 4.3 Hz, 1H), 3.46 (d, | |
| | J = 9.4 Hz, 2H), 3.38-3.33 (m, 2H), 3.30-3.17 (m, | |
| | 3H), 3.08 (t, J = 4.7 Hz, 4H), 3.05-2.82 (m, 3H), | |
| | 2.70 (s, 3H), 2.65-2.53 (m, 5H), 2.48-2.44 (m, | |
| | 1H), 2.35 (d, J = 6.8 Hz, 2H), 2.01 (ddt, J = 12.5, | |
| | 7.3, 4.4 Hz, 1H), 1.80 (s, 2H), 1.74 (td, J = 12.2, 4.1 | |
| | Hz, 1H), 1.66 (s, 1H), 1.56 (s, 1H), 0.76 (tt, J = 6.5, | |
| | 3.3 Hz, 1H). | |
| 39 | ¹H NMR (500 MHz, DMSO-d₆) δ 11.20 (s, 1H), | LCMS: |
| | 11.06 (s, 1H), 7.75 (s, 1H), 7.67-7.60 (m, 2H), | C₄₄H₅₁N₁₁O₆ |
| | 7.50 (d, J = 8.2 Hz, 2H), 7.33 (s, 1H), 7.16 (d, J = | requires: 829, |
| | 8.2 Hz, 2H), 6.92 (s, 1H), 6.82 (d, J = 8.3 Hz, 1H), | found: m/z = 830 |
| | 5.05 (dd, J = 12.9, 5.4 Hz, 1H), 4.31 (dd, J = 31.1, | [M + H]⁺. |
| | 12.9 Hz, 2H), 3.67 (d, J = 10.2 Hz, 2H), 3.61 (tt, J = | |
| | 10.1, 4.1 Hz, 1H), 3.46 (d, J = 9.5 Hz, 2H), 3.36 (d, | |
| | J = 8.3 Hz, 1H), 3.29-3.24 (m, 3H), 3.12-2.83 | |
| | (m, 5H), 2.71 (s, 3H), 2.56 (td, J = 14.9, 14.4, 4.1 | |
| | Hz, 2H), 2.42 (d, J = 11.9 Hz, 1H), 2.36 (s, 3H), | |
| | 2.09 (s, 2H), 2.00 (dt, J = 12.0, 5.3 Hz, 1H), 1.85- | |
| | 1.70 (m, 4H), 1.70-1.51 (m, 5H), 0.75 (s, 1H). | |
| 40 | ¹H NMR (500 MHz, DMSO-d₆) δ 11.07 (s, 1H), | LCMS: |
| | 10.98 (s, 1H), 7.70 (d, J = 2.9 Hz, 1H), 7.67-7.58 | C₄₄H₅₄N₁₂O₆ |
| | (m, 2H), 7.42 (d, J = 8.8 Hz, 2H), 7.33-7.20 (m, | requires: 846, |
| | 3H), 6.87 (d, J = 8.8 Hz, 2H), 5.06 (dd, J = 12.8, 5.4 | found: m/z = 847 |
| | Hz, 1H), 4.35 (d, J = 12.5 Hz, 1H), 4.27 (d, J = 13.3 | [M + H]⁺. |
| | Hz, 1H), 4.04 (d, J = 13.1 Hz, 2H), 3.60 (ddt, J = | |
| | 11.1, 8.4, 4.3 Hz, 1H), 3.35 (dd, J = 9.9, 5.9 Hz, | |
| | 2H), 3.30-3.21 (m, 5H), 3.09-2.98 (m, 4H), 3.00- | |
| | 2.82 (m, 4H), 2.70 (s, 3H), 2.65-2.53 (m, 3H), | |
| | 2.37 (t, J = 7.4 Hz, 2H), 2.01 (ddt, J = 11.3, 6.3, 4.0 | |
| | Hz, 1H), 1.84-1.70 (m, 5H), 1.64 (s, 2H), 1.43 (q, | |
| | J = 7.2 Hz, 2H), 1.26-1.15 (m, 3H). | |
| 41 | ¹H NMR (500 MHz, DMSO-d₆) δ 11.19 (s, 1H), | LCMS: |
| | 11.07 (s, 1H), 7.75 (d, J = 2.9 Hz, 1H), 7.65 (d, J = | C₄₅H₅₅N₁₁O₆ |
| | 7.7 Hz, 2H), 7.49 (d, J = 8.3 Hz, 2H), 7.34-7.28 | requires: 845, |
| | (m, 2H), 7.23 (d, J = 8.9 Hz, 1H), 7.15 (d, J = 8.2 | found: m/z = 846 |
| | Hz, 2H), 5.06 (dd, J = 12.8, 5.4 Hz, 1H), 4.35 (d, J = | [M + H]⁺. |
| | 12.4 Hz, 1H), 4.28 (d, J = 13.3 Hz, 1H), 4.04 (d, J = | |
| | 13.0 Hz, 2H), 3.62 (dq, J = 11.0, 6.3, 5.1 Hz, 1H), | |
| | 3.38-3.35 (m, 2H), 3.28-3.22 (m, 4H), 3.06- | |
| | 2.93 (m, 5H), 2.95-2.84 (m, 2H), 2.71 (s, 3H), 2.65- | |
| | 2.53 (m, 2H), 2.46-2.35 (m, 3H), 2.00 (s, 2H), | |
| | 1.86-1.71 (m, 6H), 1.60 (p, J = 10.6, 9.1 Hz, 4H), | |
| | 1.43 (t, J = 7.6 Hz, 2H), 1.20 (qd, J = 12.5, 3.9 Hz, | |
| | 2H). | |
| 42 | ¹H NMR (500 MHz, DMSO-d₆) δ 11.07 (s, 1H), | LCMS: |
| | 11.00 (s, 1H), 7.72 (d, J = 2.9 Hz, 1H), 7.68-7.60 | C₄₂H₅₀N₁₂O₆ |
| | (m, 2H), 7.44 (d, J = 8.8 Hz, 2H), 7.29 (d, J = 2.9 | requires: 818, |
| | Hz, 1H), 6.94-6.87 (m, 3H), 6.83 (dd, J = 8.6, 2.2 | found: m/z = 819 |
| | Hz, 1H), 5.06 (dd, J = 12.9, 5.4 Hz, 1H), 4.36 (d, J = | [M + H]⁺. |
| | 12.3 Hz, 1H), 4.28 (d, J = 13.3 Hz, 1H), 3.65-3.55 | |
| | (m, 1H), 3.54-3.48 (m, 1H), 3.47-3.20 (m, 5H), | |
| | 3.17 (dd, J = 10.3, 6.9 Hz, 1H), 3.13-3.07 (m, 4H), | |
| | 3.06-2.83 (m, 3H), 2.76-2.31 (m, 14H), 2.20- | |
| | 2.12 (m, 1H), 2.07-1.98 (m, 1H), 1.87-1.70 (m, | |
| | 3H), 1.59-1.50 (m, 1H). | |
| 43 | ¹H NMR (500 MHz, DMSO-d₆) δ 11.07 (s, 1H), | LCMS: |
| | 10.99 (s, 1H), 7.72 (d, J = 2.8 Hz, 1H), 7.67-7.60 | C₄₂H₅₀N₁₂O₆ |
| | (m, 2H), 7.43 (d, J = 8.9 Hz, 2H), 7.31-7.26 (m, | requires 818, |
| | 1H), 6.89 (d, J = 9.0 Hz, 2H), 6.77 (d, J = 2.1 Hz, | found: m/z = 819 |
| | 1H), 6.64 (dd, J = 8.3, 2.1 Hz, 1H), 5.06 (dd, J = | [M + H]⁺. |
| | 12.8, 5.4 Hz, 1H), 4.36 (d, J = 12.6 Hz, 1H), 4.28 (d, | |
| | J = 13.3 Hz, 1H), 4.16 (t, J = 8.2 Hz, 2H), 3.71 (dd, | |
| | J = 8.3, 5.7 Hz, 2H), 3.66-3.58 (m, 1H), 3.39- | |
| | 3.20 (m, 4H), 3.12-2.78 (m, 8H), 2.71 (s, 3H), 2.65- | |
| | 2.41 (m, 5H), 2.38-2.32 (m, 2H), 2.07-1.94 (m, | |
| | 1H), 1.90-1.70 (m, 6H), 1.59-1.53 (m, 1H). | |
| 44 | ¹H NMR (500 MHz, DMSO-d₆) δ 11.19 (s, 1H), | LCMS: |
| | 11.07 (s, 1H), 7.76 (d, J = 2.8 Hz, 1H), 7.70-7.63 | C₄₃H₅₁N₁₁O₆ |
| | (m, 2H), 7.51 (d, J = 8.5 Hz, 2H), 7.34 (d, J = 2.7 | requires: 817, |
| | Hz, 1H), 7.18 (d, J = 8.5 Hz, 2H), 6.91 (d, J = 2.2 | found: m/z = 818 |
| | Hz, 1H), 6.83 (dd, J = 8.6, 2.2 Hz, 1H), 5.07 (dd, J = | [M + H]⁺. |
| | 12.9, 5.4 Hz, 1H), 4.38 (d, J = 12.3 Hz, 1H), 4.30 (d, | |
| | J = 13.2 Hz, 1H), 3.69-3.47 (m, 2H), 3.33 (s, 4H), | |
| | 3.18 (dd, J = 10.3, 6.8 Hz, 1H), 3.10-2.82 (m, 3H), | |
| | 2.75 (s, 3H), 2.51 (p, J = 1.8 Hz, 9H), 2.19-1.97 | |
| | (m, 4H), 1.90-1.47 (m, 10H). | |
| 45 | ¹H NMR (500 MHz, DMSO-d₆) δ 11.20 (s, 1H), | LCMS: |
| | 11.07 (s, 1H), 7.76 (d, J = 2.7 Hz, 1H), 7.69-7.62 | C₄₃H₅₁N₁₁O₆ |
| | (m, 2H), 7.57-7.48 (m, 2H), 7.34 (d, J = 2.9 Hz, | requires: 817, |
| | 1H), 7.17 (d, J = 8.5 Hz, 2H), 6.77 (d, J = 2.1 Hz, | found: m/z = 818 |
| | 1H), 6.64 (dd, J = 8.4, 2.1 Hz, 1H), 5.05 (dd, J = | [M + H]⁺. |
| | 13.0, 5.3 Hz, 1H), 4.37 (d, J = 12.3 Hz, 1H), 4.30 (d, | |
| | J = 13.2 Hz, 1H), 4.16 (t, J = 8.2 Hz, 2H), 3.70 (dd, | |
| | J = 8.4, 5.7 Hz, 2H), 3.67-3.57 (m, 1H), 3.40- | |
| | 3.23 (m, 4H), 3.10-2.75 (m, 5H), 2.73 (s, 3H), 2.66- | |
| | 2.36 (m, 6H), 2.33 (t, J = 7.1 Hz, 2H), 2.05-1.94 | |
| | (m, 3H), 1.88-1.69 (m, 7H), 1.67-1.56 (m, 1H). | |
| 46 | ¹H NMR (500 MHz, DMSO-d₆) δ 11.08 (s, 1H), | LCMS: |
| | 10.99 (s, 1H), 7.74-7.70 (m, 1H), 7.67 (d, J = 8.5 | C₄₃H₅₂N₁₂O₆ |
| | Hz, 1H), 7.62 (s, 1H), 7.44 (d, J = 8.5 Hz, 2H), 7.29 | requires: 832, |
| | (d, J = 4.4 Hz, 2H), 7.22 (dd, J = 8.6, 2.3 Hz, 1H), | found: m/z = 833 |
| | 6.90 (d, J = 8.5 Hz, 2H), 5.07 (dd, J = 13.0, 5.3 Hz, | [M + H]⁺. |
| | 1H), 4.32 (dd, J = 41.4, 13.0 Hz, 2H), 3.89 (dd, J = | |
| | 24.1, 13.2 Hz, 2H), 3.62 (dd, J = 12.1, 7.9 Hz, 1H), | |
| | 3.27 (dt, J = 17.7, 7.6 Hz, 2H), 3.12 (t, J = 4.9 Hz, | |
| | 5H), 3.06-2.81 (m, 4H), 2.70 (s, 3H), 2.40-2.29 | |
| | (m, 1H), 2.18 (dd, J = 12.2, 5.8 Hz, 1H), 2.08-1.45 | |
| | (m, 7H). | |
| 47 | ¹H NMR (500 MHz, DMSO-d₆) δ 11.22 (s, 1H), | LCMS |
| | 11.09 (s, 1H), 7.76 (d, J = 2.7 Hz, 1H), 7.74-7.63 | C₃₈H₄₂N₁₀O₆ |
| | (m, 2H), 7.52 (d, J = 8.5 Hz, 2H), 7.43-7.27 (m, | requires: 735, |
| | 3H), 7.16 (d, J = 8.3 Hz, 2H), 5.09 (dd, J = 13.0, 5.4 | found: m/z = 736 |
| | Hz, 1H), 4.42-4.17 (m, 4H), 3.60 (d, J = 11.1 Hz, | [M + H]⁺. |
| | 1H), 3.26-3.16 (m, 3H), 3.16-3.04 (m, 2H), 3.04- | |
| | 2.78 (m, 5H), 2.61 (s, 5H), 2.07-1.98 (m, 1H), | |
| | 1.91-1.71 (m, 5H), 1.71-1.46 (m, 3H). | |
| 48 | ¹H NMR (500 MHz, DMSO-d₆) δ 11.10 (d, J = 8.5 | LCMS |
| | Hz, 2H), 9.37 (s, 1H), 7.82-7.66 (m, 2H), 7.65 (s, | C₄₃H₅₂N₁₂O₆ |
| | 1H), 7.60-7.46 (m, 2H), 7.41 (d, J = 2.3 Hz, 1H), | requires: 833, |
| | 7.39-7.27 (m, 2H), 6.98 (d, J = 8.7 Hz, 2H), 5.09 | found: m/z = 833.3 |
| | (dd, J = 12.7, 5.4 Hz, 1H), 4.33 (dd, J = 26.6, 12.8 | [M + H]⁺. |
| | Hz, 2H), 4.02 (dd, J = 63.9, 12.6 Hz, 2H), 3.73 (t, | |
| | J = 13.8 Hz, 4H), 3.33-3.07 (m, 10H), 3.07-2.77 | |
| | (m, 7H), 2.72 (s, 3H), 2.18 (d, J = 10.2 Hz, 1H), | |
| | 2.10-1.96 (m, 1H), 1.96-1.88 (m, 1H), 1.86- | |
| | 1.76 (m, 4H), 1.69-1.53 (m, 2H), 1.34 (q, J = 11.7 | |
| | Hz, 1H). | |
| 49 | ¹H NMR (500 MHz, DMSO-d₆) δ 11.08 (s, 1H), | LCMS |
| | 11.00 (s, 1H), 7.72 (s, 1H), 7.66 (d, J = 8.6 Hz, 1H), | C₄₃H₅₂N₁₂O₆ |
| | 7.62 (s, 1H), 7.46-7.37 (m, 2H), 7.32 (d, J = 2.3 | requires: 833, |
| | Hz, 1H), 7.31-7.22 (m, 2H), 6.92-6.86 (m, 2H), | found: m/z = 833.6 |
| | 5.77 (s, 1H), 5.07 (dd, J = 12.9, 5.4 Hz, 1H), 4.36 | [M + H]⁺. |
| | (d, J = 12.4 Hz, 1H), 4.28 (d, J = 13.0 Hz, 1H), 4.06 | |
| | (d, J = 13.2 Hz, 2H), 3.62 (s, 1H), 3.42-3.32 (m, | |
| | 4H), 3.28 (dd, J = 13.1, 7.0 Hz, 2H), 3.08 (s, 3H), | |
| | 3.06-2.85 (m, 4H), 2.71 (s, 3H), 2.63-2.54 (m, | |
| | 2H), 2.21 (d, J = 7.0 Hz, 2H), 2.02 (d, J = 12.2 Hz, | |
| | 1H), 1.83 (d, J = 13.2 Hz, 8H), 1.56 (d, J = 12.2 Hz, | |
| | 1H), 1.21 (dd, J = 24.7, 13.6 Hz, 2H), 1.10 (t, J = | |
| | 7.0 Hz, 4H). | |
| 50 | ¹H NMR (500 MHz, DMSO-d₆) δ 11.20 (s, 1H), | LCMS |
| | 11.07 (s, 1H), 7.77 (s, 1H), 7.67 (t, J = 4.3 Hz, 2H), | C₄₄H₅₃N₁₁O₆ |
| | 7.52 (d, J = 8.1 Hz, 2H), 7.32 (d, J = 24.4 Hz, 2H), | requires: 832, |
| | 7.21 (t, J = 9.3 Hz, 3H), 5.07 (dd, J = 12.9, 5.4 Hz, | found: m/z = 833 |
| | 1H), 4.34 (dd, J = 40.2, 12.9 Hz, 2H), 3.90 (s, 2H), | [M + H]⁺. |
| | 3.62 (d, J = 10.6 Hz, 2H), 3.07-2.78 (m, 10H), | |
| | 2.73 (s, 4H), 1.88-1.65 (m, 11H), 1.57 (d, J = 20.1 | |
| | Hz, 2H), 1.28 (d, J = 11.0 Hz, 2H). | |
| 51 | ¹H NMR (500 MHz, DMSO-d₆) δ 11.20 (s, 1H), | LCMS |
| | 11.08 (s, 1H), 7.76 (s, 1H), 7.71-7.61 (m, 2H), | C₄₄H₅₃N₁₁O₆ |
| | 7.54-7.48 (m, 2H), 7.37-7.30 (m, 2H), 7.25 (dd, J = | requires: 832, |
| | 8.9, 2.4 Hz, 1H), 7.18 (d, J = 8.5 Hz, 2H), 5.08 | found: m/z = 833 |
| | (dd, J = 12.8, 5.4 Hz, 1H), 4.34 (dd, J = 39.9, 12.8 | [M + H]⁺. |
| | Hz, 2H), 4.06 (d, J = 13.1 Hz, 2H), 3.62 (d, J = 11.3 | |
| | Hz, 1H), 3.11-2.80 (m, 8H), 2.73 (s, 4H), 2.69- | |
| | 2.55 (m, 3H), 2.20 (s, 2H), 2.09-1.96 (m, 3H), 1.88- | |
| | 1.70 (m, 9H), 1.70-1.48 (m, 4H), 1.17 (d, J = | |
| | 12.6 Hz, 2H). | |
| 52 | ¹H NMR (500 MHz, DMSO-d₆) δ 11.19 (s, 1H), | LCMS |
| | 11.08 (s, 1H), 7.76 (d, J = 2.8 Hz, 1H), 7.66 (d, J = | C₄₃H₅₁N₁₁O₆ |
| | 9.5 Hz, 2H), 7.51 (d, J = 8.2 Hz, 2H), 7.34 (d, J = | requires: 818, |
| | 3.0 Hz, 1H), 7.18 (d, J = 8.2 Hz, 2H), 6.91 (d, J = | found: m/z = 819 |
| | 2.2 Hz, 1H), 6.82 (dd, J = 8.5, 2.2 Hz, 1H), 5.07 (dd, | [M + H]⁺. |
| | J = 12.9, 5.4 Hz, 1H), 4.38 (d, J = 12.5 Hz, 1H), | |
| | 4.29 (d, J = 13.3 Hz, 1H), 3.57 (dddd, J = 38.9, | |
| | 22.7, 9.5, 5.5 Hz, 4H), 3.17 (dd, J = 10.4, 6.7 Hz, | |
| | 2H), 3.11-2.82 (m, 6H), 2.75 (s, 3H), 2.68-2.56 | |
| | (m, 3H), 2.44-2.34 (m, 3H), 2.20-2.05 (m, 2H), | |
| | 1.91-1.46 (m, 10H). | |
| 53 | ¹H NMR (500 MHz, DMSO-d₆) δ 11.30 (s, 1H), | LCMS |
| | 11.10 (s, 1H), 10.08 (s, 1H), 7.86-7.74 (m, 2H), | C₄₁H₄₇N₁₁O₆ |
| | 7.69 (s, 1H), 7.58 (d, J = 8.3 Hz, 2H), 7.36 (s, 1H), | requires: 790, |
| | 7.19 (d, J = 8.2 Hz, 2H), 6.95 (s, 1H), 6.80 (d, J = | found: m/z = 791 |
| | 8.3 Hz, 1H), 5.09 (dd, J = 12.8, 5.3 Hz, 1H), 4.36 (d, | [M + H]⁺. |
| | J = 44.5 Hz, 7H), 2.73 (s, 3H), 2.08 (d, J = 37.7 Hz, | |
| | 3H), 1.90-1.72 (m, 6H), 1.58 (d, J = 13.4 Hz, 1H). | |
| 54 | ¹H NMR (500 MHz, DMSO-d₆) δ 11.20 (s, 1H), | LCMS: |
| | 11.08 (s, 1H), 7.81-7.72 (m, 1H), 7.69-7.61 (m, | C₄₂H₄₉N₁₁O₆ |
| | 2H), 7.51 (d, J = 8.2 Hz, 2H), 7.34 (d, J = 2.8 Hz, | requires: 804, |
| | 1H), 7.18 (d, J = 8.5 Hz, 2H), 6.79 (d, J = 2.1 Hz, | found: m/z = 805 |
| | 1H), 6.66 (dd, J = 8.3, 2.1 Hz, 1H), 5.07 (dd, J = | [M + H]⁺. |
| | 12.8, 5.4 Hz, 1H), 4.33 (dd, J = 34.4, 12.7 Hz, 2H), | |
| | 4.16 (t, J = 8.2 Hz, 2H), 3.81-3.68 (m, 2H), 3.63 (t, | |
| | J = 10.9 Hz, 1H), 3.09-2.82 (m, 7H), 2.73 (s, 3H), | |
| | 2.68-2.59 (m, 4H), 2.05 (ddd, 20.5, 11.7, 8.1 | |
| | Hz, 3H), 1.92-1.71 (m, 6H), 1.60 (q, J = 12.2 Hz, | |
| | 4H). | |
| 55 | ¹H NMR (500 MHz, DMSO-d₆) δ 11.09 (s, 2H), | LCMS: |
| | 7.81 (s, 2H), 7.75-7.59 (m, 4H), 7.43-7.32 (m, | C₄₂H₄₉N₁₁O₆ |
| | 4H), 7.27 (dd, J = 8.7, 2.3 Hz, 2H), 5.08 (dd, J = | requires: 803, |
| | 12.8, 5.4 Hz, 2H), 4.34 (dd, J = 24.7, 13.0 Hz, 5H), | found: m/z = 804 |
| | 3.58 (dt, J = 52.0, 4.9 Hz, 9H), 3.43-3.20 (m, 8H), | [M + H]⁺. |
| | 3.14-2.81 (m, 6H), 2.72 (s, 5H), 2.67-2.54 (m, | |
| | 3H), 2.03 (ddd, J = 12.6, 5.7, 3.1 Hz, 2H), 1.92- | |
| | 1.48 (m, 30H). | |
| 56 | ¹H NMR (500 MHz, DMSO-d₆) δ 11.13 (s, 2H), | LCMS: |
| | 11.03 (s, 1H), 9.00 (s, 1H), 7.76 (s, 1H), 7.70-7.59 | C₄₆H₅₉N₁₁O₅ |
| | (m, 2H), 7.56-7.46 (m, 4H), 7.32 (s, 1H), 5.17 (dd, | requires: 845, |
| | J = 13.3, 5.1 Hz, 1H), 4.55-4.24 (m, 4H), 3.63 (dq, | found: m/z = 846 |
| | J = 11.2, 5.3, 4.6 Hz, 1H), 3.43-2.88 (m, 18H), | [M + H]⁺. |
| | 2.71 (s, 5H), 2.67-2.59 (m, 1H), 2.44-2.33 (m, | |
| | 1H), 2.04 (ddd, J = 7.4, 5.3, 2.4 Hz, 1H), 1.92-1.42 | |
| | (m, 17H). | |
| 57 | ¹H NMR (500 MHz, DMSO-d₆) δ 11.00 (s, 2H), | LCMS: |
| | 7.76 (d, J = 48.5 Hz, 6H), 7.59 (dd, J = 5.1, 3.5 Hz, | C₄₈H₆₁N₁₁O₇ |
| | 2H), 7.48 (d, J = 3.4 Hz, 4H), 5.16 (dd, J = 13.3, 5.2 | requires: 903, |
| | Hz, 2H), 4.53-4.26 (m, 6H), 3.61 (q, J = 6.2 Hz, | found: m/z = 904 |
| | 5H), 3.53-3.19 (m, 22H), 3.13-2.87 (m, 5H), 2.77- | [M + H]⁺. |
| | 2.55 (m, 15H), 2.09-1.28 (m, 34H). | |
| 58 | ¹H NMR (500 MHz, DMSO-d₆) δ 11.25 (s, 1H), | LCMS |
| | 11.08 (s, 1H), 7.97 (s, 1H), 7.83-7.72 (m, 1H), | C₃₉H₄₄N₁₀O₆ |
| | 7.67 (d, J = 9.2 Hz, 2H), 7.57 (d, J = 8.4 Hz, 2H), | requires: 749, |
| | 7.34 (d, J = 7.5 Hz, 4H), 7.26 (dd, J = 8.6, 2.4 Hz, | found: m/z = 750 |
| | 1H), 5.07 (dd, J = 12.9, 5.4 Hz, 1H), 4.35 (dd, J = | [M + H]⁺. |
| | 56.3, 12.9 Hz, 2H), 3.70-3.52 (m, 4H), 3.00 (dt, J = | |
| | 26.6, 12.2 Hz, 3H), 2.90 (s, 7H), 2.73 (d, J = 12.7 | |
| | Hz, 9H), 2.15-1.95 (m, 4H), 1.79 (dt, J = 20.2, | |
| | 13.5 Hz, 6H), 1.69-1.46 (m, 2H). | |
| 59 | ¹H NMR (500 MHz, DMSO-d₆) δ 11.09 (d, J = 14.6 | LCMS |
| | Hz, 2H), 9.46 (s, 1H), 7.83-7.68 (m, 2H), 7.64 (s, | C₄₂H₅₀N₁₂O₆ |
| | 1H), 7.51 (d, J = 8.8 Hz, 2H), 7.30 (s, 1H), 7.02- | requires: 819, |
| | 6.91 (m, 3H), 6.85 (dd, J = 8.7, 2.2 Hz, 1H), 5.06 | found: m/z = 820 |
| | (dd, J = 12.8, 5.4 Hz, 1H), 4.31 (dd, J = 26.3, 12.6 | [M + H]⁺. |
| | Hz, 2H), 3.86-3.54 (m, 12H), 3.34-3.15 (m, | |
| | 13H), 3.13-2.81 (m, 9H), 2.71 (s, 4H), 2.59 (d, J = | |
| | 18.6 Hz, 3H), 2.07-1.95 (m, 1H), 1.90-1.75 (m, | |
| | 5H), 1.67-1.51 (m, 1H). | |
| 60 | ¹H NMR (500 MHz, DMSO-d₆) δ 11.09 (d, J = 12.6 | LCMS |
| | Hz, 2H), 9.71 (s, 1H), 7.74 (s, 1H), 7.69 (d, J = 8.2 | C₄₁H₄₈N₁₂O₆ |
| | Hz, 1H), 7.64 (s, 1H), 7.51 (d, J = 8.9 Hz, 2H), 7.30 | requires: 805, |
| | (s, 1H), 6.97 (d, J = 8.9 Hz, 2H), 6.83 (d, J = 2.1 Hz, | found: m/z = 806 |
| | 1H), 6.70 (dd, J = 8.4, 2.1 Hz, 1H), 5.07 (dd, J = | [M + H]⁺. |
| | 12.8, 5.5 Hz, 1H), 4.27 (t, J = 8.2 Hz, 4H), 3.87 (dd, | |
| | J = 8.5, 5.7 Hz, 3H), 3.39-3.17 (m, 11H), 3.12- | |
| | 2.81 (m, 7H), 2.71 (s, 4H), 2.67-2.57 (m, 2H), 2.02 | |
| | (dq, J = 9.7, 4.5, 4.0 Hz, 1H), 1.88-1.73 (m, 3H), | |
| | 1.64-1.50 (m, 1H). | |
| 61 | ¹H NMR (500 MHz, DMSO-d₆) δ 10.94 (d, J = 33.3 | LCMS: |
| | Hz, 2H), 7.65 (s, 1H), 7.55 (d, J = 2.2 Hz, 1H), 7.48 | C₄₃H₅₃N₁₁O₇ |
| | (d, J = 1.4 Hz, 1H), 7.45-7.35 (m, 4H), 7.22 (s, | requires: 835, |
| | 1H), 6.86 (d, J = 8.3 Hz, 2H), 5.03 (dd, J = 13.3, 5.1 | found: m/z = 836 |
| | Hz, 1H), 4.39-4.12 (m, 5H), 3.55 (t, J = 6.4 Hz, | [M + H]⁺. |
| | 8H), 3.36-3.13 (m, 6H), 3.09-2.77 (m, 7H), 2.72- | |
| | 2.49 (m, 7H), 1.98-1.36 (m, 9H). | |
| 62 | ¹H NMR (500 MHz, Methanol-d₄) δ 7.89 (q, J = 2.2 | LCMS |
| | Hz, 1H), 7.75 (d, J = 8.5 Hz, 1H), 7.61 (s, 1H), 7.51- | C₄₁H₄₈N₁₂O₇ |
| | 7.39 (m, 2H), 7.34 (dd, J = 8.4, 2.4 Hz, 1H), 7.23 | requires: 821, |
| | (t, J = 8.1 Hz, 1H), 7.12-7.01 (m, 1H), 5.09 (dd, J = | found: m/z = 822 |
| | 12.4, 5.5 Hz, 1H), 4.34 (dd, J = 38.9, 13.0 Hz, | [M + H]⁺. |
| | 3H), 4.17 (s, 1H), 3.79-3.68 (m, 2H), 3.46-3.34 | |
| | (m, 6H), 3.03 (t, J = 12.2 Hz, 2H), 2.86 (ddd, J = | |
| | 17.9, 14.2, 5.3 Hz, 2H), 2.77 (s, 4H), 2.75-2.69 (m, | |
| | 1H), 2.66 (t, J = 6.5 Hz, 2H), 2.13 (tt, J = 10.2, 5.9 | |
| | Hz, 3H), 1.96-1.80 (m, 3H), 1.66 (q, J = 12.6, 11.9 | |
| | Hz, 1H). | |
| 63 | ¹H NMR (500 MHz, Methanol-d₄) δ 8.00 (d, J = | LCMS |
| | 18.8 Hz, 1H), 7.79-7.70 (m, 2H), 7.70-7.57 (m, | C₄₁H₄₆N₁₀O₈ |
| | 2H), 7.28 (ddd, J = 8.1, 2.1, 1.1 Hz, 1H), 7.20 (t, J = | requires: 807, |
| | 8.0 Hz, 1H), 7.14-7.02 (m, 1H), 5.12 (ddd, J = | found: m/z = 808 |
| | 12.6, 5.5, 1.4 Hz, 1H), 4.38 (dd, J = 40.7, 13.3 Hz, | [M + H]⁺. |
| | 2H), 3.92 (p, J = 7.6 Hz, 1H), 3.72 (ddd, J = 14.8, | |
| | 10.5, 4.0 Hz, 1H), 3.41 (ddt, J = 10.2, 7.1, 3.0 Hz, | |
| | 4H), 3.35-3.25 (m, 31H), 3.18 (dd, J = 9.2, 6.1 Hz, | |
| | 4H), 3.01 (t, J = 12.1 Hz, 1H), 2.89-2.78 (m, 1H), | |
| | 2.79-2.66 (m, 6H), 2.47 (dt, J = 13.8, 6.7 Hz, 2H), | |
| | 2.31-2.07 (m, 3H), 2.01-1.86 (m, 4H), 1.81 (qd, J = | |
| | 12.8, 11.7, 4.8 Hz, 1H), 1.67 (dt, J = 16.0, 11.9 | |
| | Hz, 1H). | |
| 64 | ¹H NMR (500 MHz, DMSO-d₆) δ 11.11-10.93 (m, | LCMS |
| | 2H), 7.66 (s, 1H), 7.56 (s, 1H), 7.50 (d, J = 8.3 Hz, | C₃₆H₄₀N₁₀O₇ |
| | 1H), 7.41 (dd, J = 8.9, 1.8 Hz, 3H), 7.19 (d, J = 42.2 | requires: 725, |
| | Hz, 2H), 6.92 (d, J = 2.1 Hz, 1H), 6.89-6.74 (m, | found: m/z = 726 |
| | 4H), 4.96 (dd, J = 12.7, 5.4 Hz, 1H), 4.23 (dd, J = | [M + H]⁺. |
| | 36.2, 13.0 Hz, 3H), 3.93 (dt, J = 45.1, 6.2 Hz, 3H), | |
| | 3.22-3.10 (m, 6H), 3.02-2.67 (m, 5H), 2.62 (d, J = | |
| | 10.8 Hz, 5H), 2.07-1.86 (m, 4H), 1.83-1.62 (m, | |
| | 5H). | |
| 65 | ¹H NMR (500 MHz, DMSO-d₆) δ 11.14 (s, 1H), | LCMS |
| | 10.99 (s, 1H), 7.69 (s, 1H), 7.59 (s, 1H), 7.49 (d, J = | C₃₆H₄₀N₁₀O₆ |
| | 8.3 Hz, 1H), 7.48-7.42 (m, 2H), 7.26 (s, 1H), 7.09 | requires: 709, |
| | (t, J = 12.2 Hz, 3H), 6.88 (d, J = 2.1 Hz, 1H), 6.77 | found: m/z = 710 |
| | (dd, J = 8.3, 2.1 Hz, 1H), 4.96 (dd, J = 12.8, 5.4 Hz, | [M + H]⁺. |
| | 1H), 4.24 (dd, J = 28.7, 12.7 Hz, 2H), 3.18-3.09 | |
| | (m, 5H), 3.02-2.76 (m, 4H), 2.59 (s, 5H), 1.98- | |
| | 1.87 (m, 1H), 1.87-1.63 (m, 6H), 1.48 (d, J = 12.4 | |
| | Hz, 1H). | |
| 66 | ¹H NMR (500 MHz, DMSO-d₆) δ 11.25 (d, J = 22.6 | LCMS |
| | Hz, 1H), 11.03 (d, J = 23.2 Hz, 1H), 7.72 (s, 1H), | C₄₃H₅₁N₁₁O₆ |
| | 7.67-7.44 (m, 4H), 7.33-7.19 (m, 3H), 7.07 (dd, J = | requires: 818, |
| | 17.4, 7.1 Hz, 1H), 6.84 (dd, J = 28.1, 8.6 Hz, 1H), | found: m/z = 819 |
| | 6.63 (d, J = 5.3 Hz, 1H), 6.60-6.31 (m, 2H), 4.99 | [M + H]⁺. |
| | (ddd, J = 23.5, 12.9, 5.5 Hz, 1H), 4.27 (d, J = 24.4 | |
| | Hz, 2H), 4.15-3.89 (m, 2H), 3.83 (s, 1H), 3.07- | |
| | 2.69 (m, 6H), 2.64 (d, J = 10.6 Hz, 5H), 2.21 (s, | |
| | 2H), 2.09-1.87 (m, 3H), 1.73 (q, J = 14.5, 13.9 Hz, | |
| | 4H), 1.51 (d, J = 10.3 Hz, 1H), 1.25 (s, 2H), 1.12 (s, | |
| | 2H). | |
| 67 | ¹H NMR (500 MHz, DMSO-d₆) δ 11.15 (s, 1H), | LCMS: |
| | 11.00 (s, 1H), 7.71 (s, 1H), 7.64 (d, J = 2.5 Hz, 1H), | C₃₉H₄₇N₉O₆ |
| | 7.58 (dd, J = 5.5, 3.2 Hz, 1H), 7.54-7.42 (m, 4H), | requires: 737, |
| | 7.29 (s, 1H), 6.99 (s, 2H), 5.15 (dd, J = 13.3, 5.1 Hz, | found: m/z = 738 |
| | 1H), 4.54-4.23 (m, 2H), 3.64 (dt, J = 22.8, 5.9 Hz, | [M + H]⁺. |
| | 11H), 3.39 (t, J = 6.2 Hz, 2H), 3.10 (d, J = 31.4 Hz, | |
| | 4H), 2.93 (ddd, J = 17.1, 13.7, 5.4 Hz, 1H), 2.66 (dt, | |
| | J = 32.6, 6.8 Hz, 5H), 2.02 (dtd, J = 12.8, 5.3, 2.3 | |
| | Hz, 1H), 1.84 (td, J = 7.6, 6.8, 3.3 Hz, 2H), 1.73- | |
| | 1.45 (m, 6H). | |
| 68 | ¹H NMR (500 MHz, DMSO-d₆) δ 11.28 (s, 1H), | LCMS: |
| | 11.07 (s, 1H), 7.73 (d, J = 2.9 Hz, 1H), 7.67-7.61 | C₃₈H₄₅N₁₃O₆ |
| | (m, 2H), 7.33 (d, J = 2.9 Hz, 1H), 6.93 (d, J = 2.1 | requires 779, |
| | Hz, 1H), 6.84 (dd, J = 8.6, 2.2 Hz, 1H), 6.43 (s, 1H), | found: m/z = 780 |
| | 5.05 (dd, J = 12.9, 5.4 Hz, 1H), 4.45 (d, J = 12.3 Hz, | [M + H]⁺ |
| | 1H), 4.28 (d, J = 13.2 Hz, 1H), 3.98 (t, J = 5.5 Hz, | |
| | 2H), 3.75-3.48 (m, 6H), 3.43 (dt, J = 10.1, 7.5 Hz, | |
| | 1H), 3.27-3.14 (m, 3H), 3.04-2.83 (m, 5H), 2.76- | |
| | 2.54 (m, 8H), 2.23-2.13 (m, 1H), 2.06-1.97 (m, | |
| | 1H), 1.86-1.69 (m, 5H), 1.58-1.51 (m, 1H). | |
| 69 | 1H NMR (500 MHz, Acetonitrile-d3) δ 11.23 (d, J = | LCMS: |
| | 5.8 Hz, 1H), 10.34 (s, 1H), 8.86 (s, 1H), 8.10 (d, J = | C₄₃H₅₁N₁₁O₅ |
| | 9.0 Hz, 1H), 7.69 (dd, J = 8.8, 2.7 Hz, 2H), 7.60 (s, | requires: 802, |
| | 1H), 7.50-7.36 (m, 2H), 7.31 (d, J = 8.4 Hz, 2H), | found: m/z = 802.7 |
| | 7.16 (dd, J = 9.1, 2.4 Hz, 1H), 7.06 (d, J = 7.5 Hz, | [M + H]+ |
| | 1H), 6.94 (d, J = 2.4 Hz, 1H), 6.47 (d, J = 7.5 Hz, | |
| | 1H), 5.85 (s, 1H), 4.67-4.27 (m, 4H), 4.27-4.14 | |
| | (m, 2H), 4.03 (dd, J = 31.8, 11.0 Hz, 4H), 3.82- | |
| | 3.68 (m, 1H), 3.52-3.20 (m, 6H), 3.15 (t, J = 6.5 | |
| | Hz, 2H), 3.13-2.99 (m, 3H), 2.99-2.86 (m, 4H), | |
| | 2.26-2.13 (m, 2H), 1.92-1.75 (m, 7H), 1.75- | |
| | 1.58 (m, 2H), 1.46-1.29 (m, 3H). | |
| 70 | ¹H NMR (500 MHz, Acetonitrile-d₃) δ 11.08 (s, | LCMS: |
| | 1H), 8.89 (s, 1H), 7.65 (d, J = 8.3 Hz, 1H), 7.60- | C₄₀H₄₄N₁₀O₇ |
| | 7.52 (m, 3H), 7.50-7.37 (m, 1H), 7.23 (d, J = 8.3 | requires: 776.9, |
| | Hz, 2H), 6.82 (d, J = 2.1 Hz, 1H), 6.66 (dd, J = 8.3, | found: m/z = 777.6 |
| | 2.2 Hz, 1H), 5.81 (s, 1H), 4.96 (dd, J = 12.3, 5.4 Hz, | [M + H]⁺ |
| | 1H), 4.47 (d, J = 13.0 Hz, 1H), 4.31 (t, J = 8.0 Hz, | |
| | 3H), 4.16 (t, J = 7.7 Hz, 2H), 3.90 (dd, J = 8.4, 5.2 | |
| | Hz, 2H), 3.83-3.52 (m, 3H), 3.38 (p, J = 6.1 Hz, | |
| | 1H), 3.19-2.92 (m, 4H), 2.84-2.61 (m, 3H), 2.61- | |
| | 2.53 (m, 1H), 2.16 (s, 27H), 2.07-2.00 (m, 3H), | |
| | 1.90-1.77 (m, 5H), 1.70 (tt, J = 12.9, 6.6 Hz, 3H). | |
| 71 | ¹H NMR (500 MHz, DMSO-d₆) δ 11.23 (s, 1H), | LCMS: |
| | 11.07 (s, 1H), 7.74-7.60 (m, 3H), 7.55 (d, J = 8.2 | C₄₄H₅₃N₁₁O₆ |
| | Hz, 3H), 7.30 (d, J = 8.3 Hz, 3H), 6.90 (d, J = 2.1 | requires: 832, |
| | Hz, 1H), 6.87-6.77 (m, 1H), 5.06 (dd, J = 12.9, 5.4 | found: m/z = 832.7 |
| | Hz, 1H), 4.36 (dd, J = 59.2, 13.0 Hz, 3H), 3.70- | [M + H]⁺. |
| | 3.61 (m, 2H), 3.55 (t, J = 8.8 Hz, 4H), 3.46-3.37 | |
| | (m, 5H), 3.14 (t, J = 8.5 Hz, 2H), 3.10-2.81 (m, | |
| | 5H), 2.73 (s, 4H), 2.39 (d, J = 62.2 Hz, 8H), 2.19- | |
| | 1.95 (m, 7H), 1.95-1.67 (m, 9H), 1.58 (d, J = 10.3 | |
| | Hz, 2H), 1.19 (s, 4H). | |
| 72 | ¹H NMR (500 MHz, DMSO-d₆) δ 11.30 (d, J = 6.9 | LCMS: |
| | Hz, 1H), 11.01 (s, 1H), 8.89 (s, 1H), 8.00 (d, 8.9 | C₄₅H₅₅N₁₁O₅ |
| | Hz, 1H), 7.80 (s, 1H), 7.69 (s, 1H), 7.58 (d, J = 8.1 | requires: 830, |
| | Hz, 2H), 7.36 (s, 1H), 7.29 (t, J = 8.3 Hz, 1H), 7.21 | found: m/z = 830.7 |
| | (t, J = 9.1 Hz, 3H), 7.00 (s, 1H), 6.49 (d, J = 7.5 Hz, | [M + H]⁺ |
| | 1H), 4.31 (d, J = 18.7 Hz, 2H), 4.03 (d, J = 12.9 Hz, | |
| | 2H), 3.64 (s, 4H), 3.08 (d, J = 11.3 Hz, 6H), 2.94 (t, | |
| | J = 12.9 Hz, 6H), 2.73 (s, 3H), 2.62 (d, J = 14.2 Hz, | |
| | 3H), 2.11-1.69 (m, 11H), 1.59 (d, J = 12.2 Hz, | |
| | 1H), 1.43-1.25 (m, 2H). | |
| 73 | ¹H NMR (500 MHz, DMSO-d₆) δ 11.26 (s, 1H), | LCMS: |
| | 11.08 (s, 1H), 7.84-7.75 (m, 1H), 7.75-7.59 (m, | C₄₂H₄₈N₁₀O₇ |
| | 2H), 7.51 (d, J = 8.2 Hz, 2H), 7.40-7.31 (m, 1H), | requires: 804.9, |
| | 7.19 (d, J = 8.2 Hz, 2H), 6.98-6.75 (m, 2H), 5.06 | found: m/z = 805.5 |
| | (dd, J = 12.9, 5.4 Hz, 1H), 4.41 (d, J = 12.8 Hz, 1H), | [M + H]⁺ |
| | 4.31 (t, J = 8.0 Hz, 3H), 3.61 (dt, J = 15.9, 8.2 Hz, | |
| | 7H), 3.16 (d, J = 10.7 Hz, 6H), 3.02 (dt, J = 20.5, | |
| | 10.8 Hz, 4H), 2.92-2.85 (m, 1H), 2.64 (d, J = 28.8 | |
| | Hz, 3H), 2.40 (d, J = 7.5 Hz, 3H), 2.15 (d, J = 6.9 | |
| | Hz, 1H), 2.14-1.97 (m, 4H), 1.83 (ddd, J = 43.1, | |
| | 32.8, 8.9 Hz, 7H), 1.63 (dd, J = 30.3, 13.7 Hz, 4H). | |
| 74 | ¹H NMR (500 MHz, DMSO-d₆) δ 11.34 (d, J = 33.7 | LCMS: |
| | Hz, 1H), 11.10 (s, 1H), 10.14 (d, J = 31.6 Hz, 1H), | C₄₂H₄₉N₁₁O₆ |
| | 7.89-7.53 (m, 5H), 7.35 (d, J = 12.0 Hz, 3H), 6.93 | requires: 803.9, |
| | (d, J = 11.3 Hz, 1H), 6.78 (t, J = 11.2 Hz, 1H), 5.09 | found: m/z = 804.7 |
| | (dd, J = 12.8, 5.4 Hz, 1H), 4.31 (t, J = 46.0 Hz, 8H), | [M + H]⁺ |
| | 3.15-2.77 (m, 4H), 2.72 (s, 4H), 2.04 (dt, J = 32.2, | |
| | 10.9 Hz, 3H), 1.82 (q, J = 13.1 Hz, 4H), 1.58 (d, J = | |
| | 12.2 Hz, 1H), 1.30 (dd, J = 80.8, 21.9 Hz, 4H). | |
| 75 | ¹H NMR (500 MHz, DMSO-d₆) δ 11.32 (s, 1H), | LCMS: |
| | 11.15 (s, 1H), 8.07-7.84 (m, 3H), 7.80 (s, 1H), | C₄₃H₅₁N₁₁O₆ |
| | 7.69 (s, 1H), 7.60 (d, J = 8.3 Hz, 2H), 7.39-7.22 | requires: 818, |
| | (m, 3H), 5.18 (dd, J = 12.8, 5.4 Hz, 1H), 4.35 (dd, | found: m/z = 818.7 |
| | J = 32.7, 13.3 Hz, 4H), 3.41-3.23 (m, 8H), 3.17- | [M + H]⁺ |
| | 2.85 (m, 7H), 2.71 (s, 4H), 2.63 (d, J = 22.5 Hz, | |
| | 2H), 2.13-2.03 (m, 2H), 1.82 (dd, J = 28.5, 10.7 | |
| | Hz, 6H), 1.67-1.52 (m, 1H), 1.24 (s, 3H). | |
| 76 | ¹H NMR (500 MHz, DMSO-d₆) δ 11.13 (s, 1H), | LCMS: |
| | 10.91 (s, 1H), 7.96-7.78 (m, 3H), 7.70 (d, J = 2.9 | C₄₀H₄₅N₁₁O₆ |
| | Hz, 1H), 7.60 (s, 1H), 7.46-7.35 (m, 2H), 7.26 (d, | requires: 775.9, |
| | J = 2.9 Hz, 1H), 6.63 (d, J = 8.9 Hz, 2H), 5.15 (dd, | found: m/z = 776.6 |
| | J = 12.8, 5.4 Hz, 1H), 4.32 (dd, J = 41.0, 12.7 Hz, | [M + H]⁺ |
| | 2H), 3.78 (s, 2H), 3.70-3.50 (m, 2H), 3.24 (dd, J = | |
| | 11.8, 5.2 Hz, 3H), 3.11-2.84 (m, 8H), 2.70 (d, J = | |
| | 10.6 Hz, 5H), 2.61 (dd, J = 18.3, 3.3 Hz, 3H), 2.45 | |
| | (d, J = 9.1 Hz, 3H), 2.13-1.94 (m, 1H), 1.87-1.68 | |
| | (m, 3H), 1.55 (d, J = 11.2 Hz, 1H). | |
| 77 | ¹H NMR (500 MHz, DMSO-d₆) δ 11.08 (s, 1H), | LCMS: |
| | 10.85 (s, 1H), 7.97 (s, 1H), 7.73-7.64 (m, 2H), | C₄₀H₄₉N₁₃O₆ |
| | 7.60 (s, 1H), 7.53 (s, 1H), 7.34 (d, J = 2.3 Hz, 1H), | requires: 807, |
| | 7.31-7.21 (m, 2H), 5.08 (dd, J = 12.8, 5.4 Hz, 1H), | found: m/z = 808 |
| | 4.34 (dd, J = 50.4, 13.0 Hz, 2H), 4.16-3.99 (m, | [M + H]⁺. |
| | 3H), 3.60 (dt, J = 10.4, 5.8 Hz, 1H), 3.13-2.80 (m, | |
| | 7H), 2.72-2.56 (m, 5H), 2.39-2.23 (m, 3H), 2.01 | |
| | (dd, J = 20.0, 10.3 Hz, 3H), 1.94-1.69 (m, 8H), | |
| | 1.54 (dd, J = 24.9, 12.3 Hz, 3H). | |
| 78 | ¹H NMR (500 MHz, DMSO-d₆) δ 11.08 (s, 1H), | LCMS: |
| | 10.86 (s, 1H), 7.98 (s, 1H), 7.74-7.68 (m, 1H), | C₃₉H₄₇N₁₃O₆ |
| | 7.65 (d, J = 8.3 Hz, 1H), 7.61 (s, 1H), 7.56 (s, 1H), | requires: 793, |
| | 7.29 (d, J = 2.9 Hz, 1H), 6.80 (d, J = 2.1 Hz, 1H), | found: m/z = 794 |
| | 6.67 (dd, J = 8.4, 2.1 Hz, 1H), 5.06 (dd, J = 12.7, | [M + H]⁺. |
| | 5.4 Hz, 1H), 4.34 (dd, J = 46.2, 13.0 Hz, 2H), 4.16 | |
| | (s, 3H), 3.79-3.54 (m, 3H), 3.14-2.81 (m, 4H), | |
| | 2.76-2.56 (m, 5H), 2.24-1.69 (m, 10H), 1.57 (d, | |
| | J = 12.8 Hz, 1H). | |
| 79 | ¹H NMR (500 MHz, DMSO-d₆) δ 11.07 (s, 1H), | LCMS: |
| | 10.87 (s, 1H), 7.80-7.64 (m, 2H), 7.58 (s, 1H), | C₃₉H₄₃N₁₁O₆ |
| | 7.39 (d, J = 8.5 Hz, 2H), 7.25 (s, 1H), 6.97 (d, J = | requires: 761.8, |
| | 2.1 Hz, 1H), 6.87 (dd, J = 8.6, 2.2 Hz, 1H), 6.55 (d, | found: m/z = 762.5 |
| | J = 8.7 Hz, 2H), 5.06 (dd, J = 12.9, 5.3 Hz, 1H), | [M + H]⁺ |
| | 4.30 (dd, J = 39.1, 12.9 Hz, 2H), 3.75 (d, J = 4.5 Hz, | |
| | 2H), 3.42 (d, J = 10.7 Hz, 4H), 3.35-3.15 (m, | |
| | 11H), 3.09-2.83 (m, 4H), 2.69 (s, 4H), 2.02 (qd, | |
| | J = 7.5, 6.9, 3.9 Hz, 1H), 1.87-1.71 (m, 3H), 1.60- | |
| | 1.47 (m, 1H). | |
| 80 | ¹H NMR (500 MHz, DMSO-d₆) δ 11.09 (s, 1H), | LCMS: |
| | 10.86 (s, 1H), 8.19 (d, J = 2.6 Hz, 1H), 7.88 (dd, J = | C₄₁H₄₈N₁₂O₆ |
| | 9.0, 2.7 Hz, 1H), 7.77-7.58 (m, 3H), 7.40-7.16 | requires: 804.9, |
| | (m, 3H), 6.79 (d, J = 9.1 Hz, 1H), 5.08 (dd, J = 12.9, | found: m/z = 805.7 |
| | 5.4 Hz, 1H), 4.27 (d, J = 11.3 Hz, 2H), 3.68-3.37 | [M + H]⁺ |
| | (m, 10H), 3.37-3.22 (m, 8H), 3.18 (d, J = 4.5 Hz, | |
| | 2H), 3.03 (t, J = 11.8 Hz, 1H), 2.99-2.83 (m, 2H), | |
| | 2.70 (s, 3H), 2.59 (td, J = 14.4, 3.9 Hz, 3H), 2.07- | |
| | 1.98 (m, 1H), 1.79 (q, J = 13.4, 10.1 Hz, 3H), 1.59 | |
| | (dt, J = 17.9, 5.7 Hz, 9H). | |
| 81 | ¹H NMR (500 MHz, Methanol-d₄) δ 8.16 (dd, J = | LCMS: |
| | 9.8, 3.2 Hz, 1H), 8.12-7.99 (m, 2H), 7.85-7.65 | C₄₁H₄₈N₁₂O₆ |
| | (m, 2H), 7.37 (d, J = 2.4 Hz, 1H), 7.24 (dd, J = 8.8, | requires: 804.9, |
| | 2.6 Hz, 1H), 5.09 (dd, J = 12.4, 5.5 Hz, 2H), 4.59 (d, | found: m/z = 805.6 |
| | J = 13.3 Hz, 1H), 4.35 (d, J = 13.6 Hz, 1H), 3.82 (q, | [M + H]⁺. |
| | J = 11.2, 9.1 Hz, 1H), 3.65-3.50 (m, 5H), 3.16 (dt, | |
| | J = 16.2, 12.9 Hz, 2H), 2.95-2.80 (m, 4H), 2.82- | |
| | 2.64 (m, 2H), 2.20-1.93 (m, 4H), 1.93-1.54 (m, | |
| | 9H). | |
| 82 | ¹H NMR (500 MHz, CD₃CN) δ 10.95 (s, 1H), 8.80 | LCMS: |
| | (s, 1H), 7.60-7.51 (m, 4H), 7.40 (s, 1H), 6.97 (d, J = | C₄₁H₅₀N₁₂O₅ |
| | 8.8 Hz, 2H), 6.53 (d, J = 10.0 Hz, 2H), 5.79 (s, | requires: 790, |
| | 1H), 5.04 (dd, J = 13.4, 5.1 Hz, 1H), 4.47-4.41 (m, | found: m/z = 791 |
| | 1H), 4.29 (dt, J = 22.6, 16.5 Hz, 3H), 4.19 (t, J = 7.8 | [M + H]⁺. |
| | Hz, 2H), 3.80-3.75 (m, 2H), 3.71 (ddt, J = 11.3, | |
| | 8.3, 4.1 Hz, 1H), 3.59 (br, 2H), 3.48 (d, J = 7.1 Hz, | |
| | 2H), 3.44-3.23 (m, 4H), 3.16 (br, 2H), 3.08-2.94 | |
| | (m, 2H), 2.84 (ddd, J = 18.4, 13.5, 5.4 Hz, 1H), 2.77 | |
| | (s, 2H), 2.76-2.74 (m, 1H), 2.40 (dp, J = 13.5, 5.4, | |
| | 5.0 Hz, 2H), 2.11 (ddd, J = 12.8, 7.6, 5.0 Hz, 1H), | |
| | 1.94-1.77 (m, 4H), 1.71-1.61 (m, 1H). | |
| 83 | ¹H NMR (500 MHz, DMSO-d₆) δ 11.08 (s, 1H), | LCMS: |
| | 10.85 (s, 1H), 7.98 (s, 1H), 7.73-7.64 (m, 2H), | C₄₁H₅₁N₁₃O₆ |
| | 7.60 (s, 1H), 7.55 (s, 1H), 7.36-7.21 (m, 3H), 5.07 | requires: 821, |
| | (dd, J = 12.8, 5.4 Hz, 1H), 4.34 (dd, J = 49.6, 13.1 | found: m/z = 822 |
| | Hz, 2H), 4.07 (t, J = 11.4 Hz, 3H), 3.61 (dt, J = | [M + H]⁺. |
| | 10.6, 6.0 Hz, 1H), 3.16-2.81 (m, 8H), 2.73-2.56 | |
| | (m, 4H), 2.19 (d, J = 6.8 Hz, 2H), 2.12-1.71 (m, | |
| | 14H), 1.27-1.09 (m, 2H). | |
| 84 | ¹H NMR (500 MHz, DMSO-d₆) δ 10.99 (s, 1H), | LCMS: |
| | 10.85 (s, 1H), 7.97 (s, 1H), 7.70 (d, J = 2.9 Hz, 1H), | C₃₈H₄₈N₁₂O₅ |
| | 7.63-7.56 (m, 2H), 7.54 (s, 1H), 7.52-7.45 (m, | requires: 752, |
| | 2H), 7.29 (d, J = 2.8 Hz, 1H), 5.15 (dd, J = 13.3, 5.1 | found: m/z = 753 |
| | Hz, 1H), 4.51 (d, J = 17.1 Hz, 1H), 4.34 (dt, J = | [M + H]⁺. |
| | 28.7, 13.7 Hz, 3H), 4.08 (td, J = 11.0, 5.5 Hz, 1H), | |
| | 3.60 (tt, J = 10.3, 4.8 Hz, 1H), 3.25 (t, J = 7.2 Hz, | |
| | 2H), 3.14-2.85 (m, 6H), 2.74-2.57 (m, 6H), 2.41- | |
| | 2.24 (m, 2H), 2.11-1.67 (m, 14H). | |
| 85 | ¹H NMR (500 MHz, DMSO-d₆) δ 11.26 (s, 1H), | LCMS: |
| | 11.08 (s, 1H), 7.77 (d, J = 2.7 Hz, 1H), 7.70-7.62 | C₄₀H₄₅N₁₁O₆ |
| | (m, 2H), 7.57-7.51 (m, 2H), 7.35 (d, J = 2.8 Hz, | requires 775, |
| | 1H), 7.28-7.23 (m, 2H), 6.78 (d, J = 2.1 Hz, 1H), | found: m/z = 776 |
| | 6.65 (dd, J = 8.4, 2.1 Hz, 1H), 5.09-4.96 (m, 1H), | [M + H]⁺ |
| | 4.39 (d, J = 12.4 Hz, 1H), 4.30 (d, J = 13.3 Hz, 1H), | |
| | 4.11 (t, J = 8.0 Hz, 2H), 3.76-3.54 (m, 5H), 3.45- | |
| | 3.17 (m, 3H), 3.13-2.93 (m, 4H), 2.92-2.68 (m, | |
| | 6H), 2.66-2.20 (m, 3H), 2.04-1.98 (m, 1H), 1.87- | |
| | 1.72 (m, 3H), 1.61-1.51 (m, 1H), 1.40-1.07 (m, | |
| | 2H). | |
| 86 | ¹H NMR (500 MHz, DMSO-d₆) δ 11.22 (s, 1H), | LCMS: |
| | 11.00 (s, 1H), 7.83-7.63 (m, 3H), 7.52 (q, J = 21.0, | C₄₂H₅₁N₁₁O₅ |
| | 15.4 Hz, 4H), 7.42-7.30 (m, 1H), 7.17 (d, J = 8.2 | requires: 789.9, |
| | Hz, 2H), 5.13 (dd, J = 13.4, 5.1 Hz, 1H), 4.46 (d, J = | found: m/z = 790.7 |
| | 17.2 Hz, 1H), 4.40-4.27 (m, 3H), 3.67-3.57 (m, | [M + H]⁺ |
| | 2H), 3.40 (d, J = 7.0 Hz, 9H), 3.18 (d, J = 5.2 Hz, | |
| | 1H), 3.08 (d, J = 14.6 Hz, 3H), 3.04-2.77 (m, 7H), | |
| | 2.73 (d, J = 6.1 Hz, 4H), 2.66-2.58 (m, 2H), 2.46- | |
| | 2.32 (m, 2H), 2.08-1.94 (m, 2H), 1.94-1.70 (m, | |
| | 7H), 1.58 (d, J = 15.9 Hz, 3H), 1.19 (t, J = 7.2 Hz, | |
| | 3H). | |
| 87 | ¹H NMR (500 MHz, DMSO-d₆) δ 11.19 (s, 1H), | LCMS: |
| | 11.08 (s, 1H), 7.76 (d, J = 2.8 Hz, 1H), 7.69-7.64 | C₄₃H₅₁N₁₁O₆ |
| | (m, 2H), 7.54-7.48 (m, 2H), 7.34 (d, J = 2.9 Hz, | requires: 817, |
| | 1H), 7.18 (d, J = 8.2 Hz, 2H), 7.04 (d, J = 9.6 Hz, | found: m/z = 818 |
| | 1H), 6.89 (d, J = 8.7 Hz, 1H), 5.06 (dd, J = 12.8, 5.4 | [M + H]⁺. |
| | Hz, 1H), 4.34 (dd, J = 41.1, 12.8 Hz, 2H), 4.16 (s, | |
| | 1H), 3.69-3.49 (m, 2H), 3.19 (s, 1H), 3.11-2.83 | |
| | (m, 4H), 2.73 (s, 3H), 2.68-2.55 (m, 2H), 2.46- | |
| | 2.29 (m, 2H), 2.25-1.93 (m, 7H), 1.90-1.49 (m, | |
| | 7H), 1.25 (s, 2H). | |
| 88 | ¹H NMR (500 MHz, DMSO-d₆) δ 11.44 (s, 1H), | LCMS: |
| | 11.00 (s, 1H), 8.08 (d, J = 9.0 Hz, 1H), 7.98 (d, J = | C₄₀H₅₀N₁₂O₅ |
| | 3.0 Hz, 1H), 7.77 (d, J = 2.7 Hz, 1H), 7.71 (s, 1H), | requires 778, |
| | 7.62-7.54 (m, 1H), 7.51-7.44 (m, 2H), 7.38- | found: m/z = 779 |
| | 7.31 (m, 2H), 5.14 (dd, J = 13.3, 5.2 Hz, 1H), 4.48 | [M + H]⁺. |
| | (d, J = 17.1 Hz, 1H), 4.42-4.28 (m, 3H), 3.68- | |
| | 3.59 (m, 1H), 3.44-3.21 (m, 5H), 3.13-2.87 (m, | |
| | 7H), 2.72-2.66 (m, 5H), 2.65-2.56 (m, 1H), 2.55- | |
| | 2.40 (m, 5H), 2.39-2.32 (m, 2H), 2.07-1.98 (m, | |
| | 1H), 1.88-1.72 (m, 3H), 1.72-1.46 (m, 4H). | |
| 89 | ¹H NMR (500 MHz, DMSO-d₆) δ 11.45 (s, 1H), | LCMS: |
| | 11.07 (s, 1H), 8.09 (d, J = 9.1 Hz, 1H), 8.01 (d, J = | C₄₁H₄₉N₁₃O₆ |
| | 3.0 Hz, 1H), 7.77 (d, J = 2.8 Hz, 1H), 7.71 (s, 1H), | requires 819, |
| | 7.65 (d, J = 8.4 Hz, 1H), 7.40-7.33 (m, 2H), 6.92 | found: m/z = 820 |
| | (d, J = 2.2 Hz, 1H), 6.83 (dd, J = 8.6, 2.2 Hz, 1H), | [M + H]⁺ |
| | 5.03 (d, J = 11.0 Hz, 1H), 4.40 (d, J = 12.5 Hz, 1H), | |
| | 4.31 (d, J = 13.3 Hz, 1H), 3.67-3.56 (m, 2H), 3.54- | |
| | 3.50 (m, 1H), 3.47-3.22 (m, 6H), 3.21-2.93 (m, | |
| | 7H), 2.87 (t, J = 14.0 Hz, 1H), 2.75-2.32 (m, 12H), | |
| | 2.20-2.13 (m, 1H), 2.04-1.97 (m, 1H), 1.90- | |
| | 1.73 (m, 3H), 1.65-1.49 (m, 2H). | |
| 90 | ¹H NMR (500 MHz, DMSO-d₆) δ 11.58 (s, 1H), | LCMS: |
| | 11.16 (s, 1H), 8.02 (d, J = 50.3 Hz, 3H), 7.86 (s, | C₃₉H₄₃N₁₁O₇ |
| | 1H), 7.76 (s, 1H), 7.69 (d, J = 8.1 Hz, 2H), 7.44 (d, | requires: 777.8, |
| | J = 7.0 Hz, 3H), 5.20 (dd, J = 12.8, 5.5 Hz, 1H), | found: m/z = 778.6 |
| | 4.35 (dd, J = 28.1, 12.6 Hz, 3H), 3.17-2.80 (m, | [M + H]⁺ |
| | 6H), 2.70 (s, 3H), 2.14-2.00 (m, 1H), 1.82 (q, J = | |
| | 11.2, 9.6 Hz, 3H), 1.59 (d, J = 12.0 Hz, 1H). | |
| 91 | ¹H NMR (500 MHz, DMSO-d₆) δ 11.07 (s, 1H), | LCMS: |
| | 10.90 (s, 1H), 7.75-7.67 (m, 1H), 7.67-7.58 (m, | C₄₃H₅₀N₁₂O₆ |
| | 2H), 7.39 (d, J = 8.8 Hz, 2H), 7.26 (d, J = 2.6 Hz, | requires: 831, |
| | 1H), 6.77 (d, J = 2.1 Hz, 1H), 6.68-6.59 (m, 3H), | found: m/z = 831.7 |
| | 5.77 (s, 1H), 5.05 (dd, J = 12.7, 5.5 Hz, 1H), 4.32 | [M + H]⁺ |
| | (dd, J = 42.2, 13.2 Hz, 2H), 4.13 (t, J = 8.2 Hz, 2H), | |
| | 3.75-3.57 (m, 3H), 3.38 (dd, J = 14.1, 7.1 Hz, 3H), | |
| | 3.08-2.77 (m, 8H), 2.70 (d, J = 14.7 Hz, 9H), 2.44- | |
| | 2.28 (m, 8H), 2.12-1.93 (m, 2H), 1.89-1.67 (m, | |
| | 4H), 1.56 (d, J = 12.1 Hz, 1H). | |
| 92 | ¹H NMR (500 MHz, DMSO-d₆) δ 11.24 (s, 1H), | LCMS: |
| | 11.08 (s, 1H), 7.78 (s, 1H), 7.67 (q, J = 10.7, 8.1 Hz, | C₄₀H₄₅N₁₁O₆ |
| | 2H), 7.57 (s, 1H), 7.36 (s, 1H), 7.16 (d, J = 8.2 Hz, | requires: 775.9, |
| | 1H), 6.98 (d, J = 8.4 Hz, 1H), 6.80 (s, 1H), 6.66 (d, | found: m/z = 776.7 |
| | J = 8.4 Hz, 1H), 5.07 (dd, J = 12.9, 5.5 Hz, 1H), | [M + H]⁺ |
| | 4.34 (dd, J = 43.8, 11.9 Hz, 2H), 4.17 (t, J = 8.4 Hz, | |
| | 2H), 3.75 (t, J = 6.8 Hz, 2H), 3.64 (d, J = 11.4 Hz, | |
| | 1H), 3.55 (s, 2H), 3.08 (dd, J = 24.9, 13.1 Hz, 2H), | |
| | 3.03-2.85 (m, 3H), 2.77 (t, J = 6.3 Hz, 3H), 2.70 | |
| | (s, 5H), 2.11-1.96 (m, 1H), 1.81 (q, J = 21.3, 17.4 | |
| | Hz, 3H), 1.59 (d, J = 14.4 Hz, 1H). | |
| 93 | ¹H NMR (500 MHz, DMSO-d₆) δ 11.13 (s, 1H), | LCMS: |
| | 10.97 (s, 1H), 7.92-7.81 (m, 2H), 7.83-7.77 (m, | C₄₁H₄₇N₁₁O₆ |
| | 2H), 7.71 (d, J = 3.0 Hz, 1H), 7.61 (s, 1H), 7.41 (d, | requires 789, |
| | J = 8.9 Hz, 2H), 7.28 (d, J = 2.9 Hz, 1H), 6.88 (d, J = | found: m/z = 790 |
| | 8.9 Hz, 2H), 5.15 (dd, J = 12.8, 5.4 Hz, 1H), 4.70 | [M + H]⁺ |
| | (s, 1H), 4.36 (d, J = 12.5 Hz, 1H), 4.27 (d, J = 13.4 | |
| | Hz, 1H), 3.81 (s, 2H), 3.69-3.55 (m, 1H), 3.41- | |
| | 3.20 (m, 2H), 3.10-2.84 (m, 11H), 2.71 (s, 3H), | |
| | 2.65-2.52 (m, 2H), 2.10-2.03 (m, 1H), 1.85- | |
| | 1.71 (m, 7H), 1.57-1.53 (m, 1H). | |
| 94 | ¹H NMR (500 MHz, DMSO-d₆) δ 11.12 (s, 1H), | LCMS: |
| | 11.02 (s, 1H), 7.88 (d, J = 7.5 Hz, 1H), 7.83-7.77 | C₄₁H₄₇N₁₁O₆ |
| | (m, 2H), 7.72 (d, J = 2.9 Hz, 1H), 7.62 (s, 1H), 7.48- | requires 789, |
| | 7.42 (m, 2H), 7.29 (d, J = 3.1 Hz, 1H), 6.91 (d, J = | found: m/z = 790 |
| | 8.9 Hz, 2H), 5.11 (s, 1H), 4.41 (d, J = 12.2 Hz, 1H), | [M + H]⁺ |
| | 4.28 (d, J = 13.5 Hz, 1H), 3.82 (s, 2H), 3.65-3.61 | |
| | (m, 1H), 3.33-3.21 (m, 3H), 3.19-3.09 (m, 4H), | |
| | 3.05-2.80 (m, 7H), 2.72 (s, 3H), 2.67-2.53 (m, | |
| | 2H), 2.10-2.00 (m, 1H), 1.89-1.48 (m, 9H). | |
| 95 | Stereoisomer 1: ¹H NMR (500 MHz, Methanol-d₄) δ | Stereoisomer 1: |
| | 8.08 (s, 1H), 7.99 (q, J = 7.2, 5.2 Hz, 2H), 7.62 (d, J = | LCMS: |
| | 92.6 Hz, 4H), 7.00 (s, 1H), 5.19 (dd, J = 12.6, 5.4 | C₄₂H₄₉N₁₁O₆ |
| | Hz, 1H), 4.55 (s, 2H), 4.40 (d, J = 14.2 Hz, 2H), | requires 804, |
| | 3.77 (dq, J = 10.4, 5.4, 4.2 Hz, 1H), 3.61 (s, 2H), | found: m/z = 804.6 |
| | 3.50 (dt, J = 10.2, 7.5 Hz, 2H), 3.45-3.39 (m, 2H), | [M + H]⁺ |
| | 3.41-3.34 (m, 4H), 3.12 (s, 2H), 3.00 (t, J = 12.8 | Stereoisomer 2: |
| | Hz, 1H), 2.90 (ddd, J = 18.5, 14.0, 5.3 Hz, 2H), 2.80 | LCMS: |
| | (s, 3H), 2.79-2.77 (m, 1H), 2.77-2.70 (m, 2H), | C₄₂H₄₉N₁₁O₆ |
| | 2.39-2.05 (m, 5H), 1.90 (dt, J = 30.1, 12.7 Hz, | requires 804, |
| | 4H), 1.75-1.59 (m, 2H). | found: m/z = 804.7 |
| | Stereoisomer 2: ¹H NMR (500 MHz, Methanol-d₄) δ | [M + H]⁺ |
| | 8.10 (s, 1H), 8.05 (d, J = 7.7 Hz, 1H), 8.01 (d, J = | |
| | 7.5 Hz, 1H), 7.95-7.81 (m, 2H), 7.63 (s, 2H), 7.17 | |
| | (s, 1H), 5.18 (ddd, J = 25.3, 12.6, 5.5 Hz, 2H), 4.66- | |
| | 4.49 (m, 2H), 4.44 (d, J = 12.6 Hz, 1H), 4.35 (d, J = | |
| | 13.7 Hz, 1H), 3.65 (d, J = 12.6 Hz, 1H), 3.53- | |
| | 3.40 (m, 4H), 3.13 (t, J = 11.8 Hz, 3H), 3.03 (t, J = | |
| | 12.8 Hz, 2H), 2.98-2.83 (m, 3H), 2.83-2.69 (m, | |
| | 7H), 2.06 (d, J = 12.9 Hz, 2H), 1.93 (d, J = 23.4 Hz, | |
| | 3H), 1.90-1.80 (m, 1H), 1.64 (d, J = 34.6 Hz, 4H). | |
| 96 | ¹H NMR (500 MHz, DMSO-d₆) δ 11.08-11.01 (m, | LCMS: |
| | 2H), 7.72 (d, J = 2.8 Hz, 1H), 7.64-7.54 (m, 2H), | C₄₀H₄₅N₁₁O₆ |
| | 7.45 (d, J = 8.8 Hz, 2H), 7.29 (d, J = 2.8 Hz, 1H), | requires 775, |
| | 7.12 (d, J = 6.9 Hz, 1H), 6.95 (d, J = 8.9 Hz, 2H), | found: m/z = 776 |
| | 6.83 (d, J = 8.5 Hz, 1H), 5.05 (dd, J = 12.8, 5.4 Hz, | [M + H]⁺ |
| | 1H), 4.39 (d, J = 12.4 Hz, 1H), 4.27 (d, J = 13.4 Hz, | |
| | 1H), 4.09-3.87 (m, 4H), 3.66-3.56 (m, 1H), 3.26- | |
| | 3.13 (m, 5H), 3.02-2.83 (m, 5H), 2.65 (s, 3H), | |
| | 2.62-2.52 (m, 2H), 2.06-1.95 (m, 1H), 1.87- | |
| | 1.63 (m, 8H), 1.58-1.52 (m, 1H). | |
| 97 | ¹H NMR (500 MHz, DMSO-d₆) δ 11.07 (s, 1H), | LCMS: |
| | 11.00 (s, 1H), 7.72 (d, J = 2.8 Hz, 1H), 7.64-7.54 | C₄₀H₄₅N₁₁O₆ |
| | (m, 2H), 7.46-7.41 (m, 2H), 7.29 (d, J = 3.0 Hz, | requires 775, |
| | 1H), 7.12 (d, J = 6.9 Hz, 1H), 6.92 (d, J = 8.9 Hz, | found: m/z = 776 |
| | 2H), 6.81 (d, J = 8.5 Hz, 1H), 5.05 (dd, J = 12.7, 5.5 | [M + H]⁺ |
| | Hz, 1H), 4.37 (d, J = 12.4 Hz, 1H), 4.28 (d, J = 13.3 | |
| | Hz, 1H), 4.00 (s, 4H), 3.64-3.60 (m, 1H), 3.33 (s, | |
| | 4H), 3.15-2.81 (m, 7H), 2.72 (s, 3H), 2.67-2.40 | |
| | (m, 2H), 2.05-1.71 (m, 8H), 1.60-1.53 (m, 1H). | |
| 98 | ¹H NMR (500 MHz, CD₃CN) δ 10.84 (s, 1H), 10.17 | LCMS: |
| | (s, 1H), 7.78 (s, 1H), 7.66 (d, J = 7.8 Hz, 1H), 7.54 | C₄₀H₄₅N₁₁O₅ |
| | (d, J = 7.9 Hz, 1H), 7.50 (s, 1H), 7.46 (d, J = 8.6 Hz, | requires: 759, |
| | 2H), 7.41 (s, 1H), 6.91 (d, J = 8.5 Hz, 2H), 6.22 (s, | found: m/z = 760 |
| | 1H), 5.06 (dd, J = 13.5, 5.2 Hz, 1H), 4.39 (q, J = | [M + H]⁺. |
| | 17.3 Hz, 3H), 4.24 (d, J = 13.8 Hz, 1H), 3.65 (d, J = | |
| | 11.9 Hz, 1H), 3.57 (s, 2H), 3.39-3.20 (m, 5H), | |
| | 3.16 (s, 4H), 2.95 (dt, J = 23.8, 12.3 Hz, 2H), 2.84- | |
| | 2.67 (m, 4H), 2.38-2.27 (m, 3H), 2.10 (dd, J = | |
| | 13.5, 7.1 Hz, 1H), 1.90-1.74 (m, 5H), 1.61 (q, J = | |
| | 12.5 Hz, 1H). | |
| 99 | ¹H NMR (500 MHz, CD₃CN) δ 10.81 (s, 1H), 8.83 | LCMS: |
| | (d, J = 40 Hz, 1H), 7.73 (d, J = 7.4 Hz, 1H), 7.65 (d, | C₄₀H₄₅N₁₁O₅ |
| | J = 7.6 Hz, 1H), 7.54-7.44 (m, 4H), 7.36 (s, 1H), | requires: 759, |
| | 6.94-6.87 (m, 2H), 5.72 (s, 1H), 5.07 (dd, J = 13.3, | found: m/z = 760 |
| | 5.2 Hz, 1H), 4.49-4.33 (m, 3H), ), 4.24 (s, 1H), | [M + H]⁺. |
| | 3.68 (s, 1H), 3.63 (d, J = 2.6 Hz, 2H), 3.37-3.22 | |
| | (m, 5H), 3.17 (t, J = 4.9 Hz, 4H), 3.03-2.90 (m, | |
| | 2H), 2.85-2.61 (m, 4H), 2.45-2.37 (m, 3H), 2.09 | |
| | (s, 1H), 1.87-1.76 (m, 5H), 1.64 (s, 1H). | |
| 100 | ¹H NMR (500 MHz, Acetonitrile-d₃) δ 11.12 (s, | LCMS: |
| | 1H), 10.62 (s, 1H), 8.96 (s, 1H), 8.10 (s, 1H), 8.05- | C₃₉H₄₄N₁₀O₆ |
| | 7.91 (m, 2H), 7.68-7.51 (m, 3H), 7.42 (s, 1H), 7.19 | requires: 748.9, |
| | (d, J = 8.3 Hz, 2H), 5.81 (s, 1H), 5.07 (dd, J = 12.4, | found: m/z = 749.6 |
| | 5.4 Hz, 1H), 4.41 (d, J = 13.4 Hz, 3H), 4.30 (d, J = | [M + H]⁺ |
| | 13.7 Hz, 1H), 3.78-3.61 (m, 1H), 3.56 (d, J = 12.2 | |
| | Hz, 2H), 3.46-3.23 (m, 5H), 3.16-2.95 (m, 5H), | |
| | 2.86-2.72 (m, 8H), 2.22-2.15 (m, 3H), 1.92- | |
| | 1.76 (m, 5H), 1.66 (d, J = 13.1 Hz, 2H). | |
| 101 | ¹H NMR (500 MHz, DMSO-d₆) δ 11.19 (s, 1H), | LCMS: |
| | 11.12 (s, 1H), 7.87 (d, J = 7.6 Hz, 1H), 7.83-7.77 | C₄₂H₄₉N₁₁O₆ |
| | (m, 2H), 7.75 (d, J = 2.7 Hz, 1H), 7.66 (s, 1H), 7.49 | requires: 813, |
| | (d, J = 8.4 Hz, 2H), 7.32 (s, 1H), 7.15 (d, J = 8.3 Hz, | found: m/z = 814 |
| | 2H), 5.14 (dd, J = 12.8, 5.4 Hz, 1H), 4.34 (d, J = | [M + H]⁺. |
| | 13.2 Hz, 1H), 4.28 (d, J = 13.2 Hz, 1H), 3.77 (s, | |
| | 2H), 3.60 (dq, J = 10.4, 5.6, 4.1 Hz, 1H), 3.42 (d, | |
| | J = 5.5 Hz, 2H), 3.37-3.32 (m, 1H), 3.30-3.18 (m, | |
| | 2H), 3.04 (t, J = 11.8 Hz, 1H), 2.99-2.83 (m, 5H), | |
| | 2.80 (d, J = 10.6 Hz, 2H), 2.71 (s, 3H), 2.63 (dt, J = | |
| | 6.8, 2.4 Hz, 1H), 2.59-2.55 (m, 1H), 2.44-2.32 | |
| | (m, 1H), 2.12-1.97 (m, 1H), 1.95-1.67 (m, 8H), | |
| | 1.58 (q, J = 12.0, 11.5 Hz, 3H). | |
| 102 | ¹H NMR (500 MHz, CD₃CN) δ 11.17 (s, 1H), 11.12 | LCMS: |
| | (s, 1H), 10.71 (s, 2H), 8.91 (s, 2H), 7.74-7.55 (m, | C₄₃H₅₁N₁₁O₆ |
| | 6H), 7.44 (s, 2H), 7.34 (dd, J = 11.6, 8.5 Hz, 3H), | requires: 817, |
| | 7.18-7.08 (m, 2H), 7.03-6.99 (m, 1H), 6.94 (d, J = | found: m/z = 818 |
| | 8.6 Hz, 1H), 6.48-6.33 (m, 2H), 5.83 (s, 2H), | [M + H]⁺. |
| | 4.96 (dd, J = 12.4, 5.4 Hz, 2H), 4.48 (s, 2H), 4.32 | |
| | (d, J = 12.8 Hz, 2H), 4.08-3.86 (m, 1H), 3.73 (s, | |
| | 0H), 3.57-3.17 (m, 5H), 3.04 (dt, J = 25.2, 11.9 | |
| | Hz, 4H), 2.81 (d, J = 15.0 Hz, 0H), 2.74-2.63 (m, | |
| | 3H), 2.48 (d, J = 53.6 Hz, 1H), 1.68 (d, J = 12.7 Hz, | |
| | 2H), 1.38 (s, 2H), 1.24 (s, 3H). | |
| 103 | ¹H NMR (500 MHz, CD₃CN) δ 11.14 (d, J = 13.1 | LCMS: |
| | Hz, 1H), 8.93 (s, 1H), 7.72-7.55 (m, 4H), 7.43 (s, | C₄₃H₅₁N₁₁O₆ |
| | 1H), 7.33 (dd, J = 17.9, 8.6 Hz, 2H), 7.14 (dd, J = | requires: 817, |
| | 14.5, 7.1 Hz, 1H), 6.87 (dd, J = 22.9, 8.5 Hz, 1H), | found: m/z = 818 |
| | 6.46 (s, 0H), 6.36 (d, J = 5.2 Hz, 0H), 5.82 (s, 1H), | [M + H]⁺. |
| | 5.05-4.87 (m, 1H), 4.48 (s, 1H), 4.33 (s, 0H), 3.72 | |
| | (s, 2H), 3.52-3.22 (m, 5H), 3.14-2.85 (m, 3H), | |
| | 2.83-2.63 (m, 5H), 2.53 (t, J = 7.6 Hz, 1H), 1.37 | |
| | (s, 1H), 1.25 (d, J = 7.2 Hz, 2H). | |
| 104 | Stereoisomer 2: ¹H NMR (500 MHz, Methanol-d₄) δ | LCMS: |
| | 8.10 (s, 1H), 8.05 (d, J = 7.7 Hz, 1H), 8.01 (d, J = | C₄₂H₄₉N₁₁O₆ |
| | 7.5 Hz, 1H), 7.95-7.81 (m, 2H), 7.63 (s, 2H), 7.17 | requires 804, |
| | (s, 1H), 5.18 (ddd, J = 25.3, 12.6, 5.5 Hz, 2H), 4.66- | found: m/z = 804.7 |
| | 4.49 (m, 2H), 4.44 (d, J = 12.6 Hz, 1H), 4.35 (d, J = | [M + H]⁺ |
| | 13.7 Hz, 1H), 3.65 (d, J = 12.6 Hz, 1H), 3.53- | |
| | 3.40 (m, 4H), 3.13 (t, J = 11.8 Hz, 3H), 3.03 (t, J = | |
| | 12.8 Hz, 2H), 2.98-2.83 (m, 3H), 2.83-2.69 (m, | |
| | 7H), 2.06 (d, J = 12.9 Hz, 2H), 1.93 (d, J = 23.4 Hz, | |
| | 3H), 1.90-1.80 (m, 1H), 1.64 (d, J = 34.6 Hz, 4H). | |
| 105 | ¹H NMR (500 MHz, Methanol-d₄) δ 7.82 (ddd, J = | LCMS: |
| | 8.1, 6.2, 3.9 Hz, 2H), 7.75-7.65 (m, 2H), 7.60 (dd, | C₄₁H₄₇N₁₁O₆ |
| | J = 16.5, 8.8 Hz, 2H), 7.47-7.32 (m, 2H), 5.20- | requires 790, |
| | 5.00 (m, 1H), 4.36 (dt, J = 50.2, 13.1 Hz, 2H), 4.15 | found: m/z = 790.7 |
| | (q, J = 13.1, 11.1 Hz, 1H), 4.03-3.63 (m, 5H), 3.57 | [M + H]⁺ |
| | (d, J = 13.3 Hz, 1H), 3.54-3.36 (m, 3H), 3.36- | |
| | 3.30 (m, 4H), 3.02 (d, J = 14.6 Hz, 3H), 2.81-2.63 | |
| | (m, 5H), 2.58 (d, J = 12.2 Hz, 1H), 2.24-2.00 (m, | |
| | 3H), 2.01-1.79 (m, 3H), 1.71 (d, J = 37.4 Hz, 2H). | |
| 106 | Stereoisomer 2: ¹H NMR (500 MHz, Methanol-d₄) δ | LCMS: |
| | 7.83 (dd, J = 9.2, 3.1 Hz, 2H), 7.72 (d, J = 2.0 Hz, | C₄₁H₄₇N₁₁O₆ |
| | 1H), 7.69 (dd, J = 8.6, 2.5 Hz, 1H), 7.56 (dd, J = | requires 790, |
| | 9.1, 3.2 Hz, 2H), 7.41 (d, J = 2.3 Hz, 1H), 7.28 (dd, | found: m/z = 790.7 |
| | J = 8.5, 2.4 Hz, 1H), 5.08 (ddd, J = 12.6, 5.6, 2.7 | [M + H]⁺ |
| | Hz, 1H), 4.45 (d, J = 12.9 Hz, 1H), 4.34 (d, J = 13.8 | |
| | Hz, 1H), 4.07 (s, 1H), 3.97 (s, 1H), 3.76 (dd, J = | |
| | 24.2, 12.2 Hz, 5H), 3.56 (d, J = 12.2 Hz, 1H), 3.52- | |
| | 3.39 (m, 4H), 3.18-2.99 (m, 5H), 2.91-2.82 (m, | |
| | 2H), 2.80-2.76 (m, 2H), 2.74 (dd, J = 5.5, 3.2 Hz, | |
| | 4H), 2.71 (s, 1H), 2.58 (s, 1H), 2.29 (s, 3H), 2.09 | |
| | (dd, J = 22.6, 14.0 Hz, 3H), 1.94 (s, 5H), 1.72 (s, | |
| | 3H). | |
| 107 | Stereoisomer 1: ¹H NMR (500 MHz, Methanol-d₄) δ | LCMS: |
| | 7.88 (d, J = 8.7 Hz, 2H), 7.75 (s, 1H), 7.72 (d, J = | C₄₁H₄₇N₁₁O₆ |
| | 8.6 Hz, 1H), 7.58 (d, J = 8.8 Hz, 2H), 7.43 (d, J = | requires 790, |
| | 2.2 Hz, 1H), 7.33-7.28 (m, 1H), 5.10 (dd, J = 12.4, | found: m/z = 790.5 |
| | 5.5 Hz, 1H), 4.47 (d, J = 12.7 Hz, 1H), 4.39 (d, J = | [M + H]⁺ |
| | 14.1 Hz, 1H), 4.21 (d, J = 13.5 Hz, 1H), 4.07 (d, J = | |
| | 12.9 Hz, 1H), 3.83-3.65 (m, 4H), 3.56-3.49 (m, | |
| | 1H), 3.46 (t, J = 7.9 Hz, 2H), 3.40 (t, J = 7.8 Hz, | |
| | 2H), 3.18-3.04 (m, 5H), 2.93-2.87 (m, 1H), 2.84 | |
| | (s, 4H), 2.81-2.67 (m, 4H), 2.18-2.06 (m, 2H), | |
| | 1.96 (t, J = 10.2 Hz, 4H), 1.91-1.85 (m, 2H), 1.51 | |
| | (d, J = 12.8 Hz, 2H), 0.92 (t, J = 6.7 Hz, 3H). | |
| 108 | ¹H NMR (500 MHz, DMSO-d₆) δ 11.17 (d, J = 7.9 | LCMS: |
| | Hz, 2H), 9.41 (s, 1H), 8.17 (s, 1H), 8.13-7.97 (m, | C₄₃H₅₁N₁₁O₆ |
| | 2H), 7.77 (s, 1H), 7.67 (s, 1H), 7.55 (d, J = 8.3 Hz, | requires: 818, |
| | 2H), 7.34 (s, 1H), 7.14 (s, 2H), 5.19 (td, J = 13.1, | found: m/z = 818.7 |
| | 5.9 Hz, 1H), 4.54 (d, J = 48.5 Hz, 3H), 4.33 (dd, J = | [M + H]⁺ |
| | 28.6, 12.0 Hz, 4H), 3.45-3.12 (m, 9H), 3.12-2.76 | |
| | (m, 6H), 2.71 (s, 3H), 2.26-1.99 (m, 2H), 1.99- | |
| | 1.73 (m, 7H), 1.58 (dd, J = 17.8, 9.0 Hz, 3H), 1.44- | |
| | 1.23 (m, 1H). | |
| 109 | ¹H NMR (500 MHz, DMSO-d₆) δ 11.07 (s, 1H), | LCMS: |
| | 10.99 (s, 1H), 7.72 (d, J = 2.7 Hz, 1H), 7.66 (d, J = | C₄₁H₄₇N₁₁O₆ |
| | 8.4 Hz, 1H), 7.62 (s, 1H), 7.44 (d, J = 8.6 Hz, 2H), | requires: 789, |
| | 7.29 (d, J = 2.8 Hz, 1H), 6.97 (d, J = 2.1 Hz, 1H), | found: m/z = 790 |
| | 6.93 (d, J = 8.7 Hz, 2H), 6.85 (dd, J = 8.7, 2.2 Hz, | [M + H]⁺ |
| | 1H), 5.06 (dd, J = 12.7, 5.3 Hz, 1H), 4.44-4.21 (m, | |
| | 2H), 3.70-3.48 (m, 3H), 3.29-3.07 (m, 3H), 3.06- | |
| | 2.80 (m, 2H), 2.74-2.60 (m, 6H), 2.07-1.91 (m, | |
| | 3H), 1.87-1.44 (m, 10H). | |
| 110 | ¹H NMR (500 MHz, DMSO-d₆) δ 11.00 (s, 1H), | LCMS: |
| | 10.87 (s, 1H), 8.20 (d, J = 2.7 Hz, 1H), 7.88 (dd, J = | C₄₀H₅₀N₁₂O₅ |
| | 9.1, 2.8 Hz, 1H), 7.73 (s, 1H), 7.64 (s, 1H), 7.61- | requires 778, |
| | 7.54 (m, 1H), 7.51-7.44 (m, 2H), 7.31 (d, J = 2.8 | found: m/z = 779 |
| | Hz, 1H), 6.77 (d, J = 9.1 Hz, 1H), 5.14 (dd, J = 13.3, | [M + H]⁺ |
| | 5.1 Hz, 1H), 4.48 (d, J = 17.2 Hz, 1H), 4.37-4.24 | |
| | (m, 3H), 3.63-3.51 (m, 1H), 3.48-3.14 (m, 7H), | |
| | 3.03 (t, J = 11.7 Hz, 1H), 2.97-2.87 (m, 2H), 2.74- | |
| | 2.22 (m, 13H), 2.05-1.99 (m, 1H), 1.82-1.77 | |
| | (m, 3H), 1.69-1.61 (m, 2H), 1.57-1.49 (m, 4H). | |
| 111 | ¹H NMR (500 MHz, DMSO-d₆) δ 11.16-10.96 (m, | LCMS: |
| | 2H), 7.72 (d, J = 2.9 Hz, 1H), 7.69-7.60 (m, 2H), | C₄₀H₄₅N₁₁O₆ |
| | 7.48-7.42 (m, 2H), 7.29 (d, J = 2.9 Hz, 1H), 6.99- | requires 775, |
| | 6.92 (m, 2H), 6.83 (d, J = 2.1 Hz, 1H), 6.70 (dd, J = | found: m/z = 776 |
| | 8.4, 2.1 Hz, 1H), 5.08-5.01 (m, 1H), 4.38 (d, J = | [M + H]⁺ |
| | 12.5 Hz, 1H), 4.27 (d, J = 13.1 Hz, 1H), 3.85 (d, J = | |
| | 9.0 Hz, 2H), 3.79 (d, J = 8.4 Hz, 2H), 3.63-3.59 | |
| | (m, 1H), 3.26-3.16 (m, 3H), 3.03-2.82 (m, 5H), | |
| | 2.65 (s, 3H), 2.61-2.35 (m, 4H), 2.05-1.98 (m, | |
| | 1H), 1.86-1.63 (m, 8H), 1.60-1.48 (m, 1H). | |
| 112 | ¹H NMR (500 MHz, DMSO-d₆) δ 11.08 (s, 1H), | LCMS: |
| | 11.00 (s, 1H), 7.72 (d, J = 3.0 Hz, 1H), 7.69-7.60 | C₄₀H₄₅N₁₁O₆ |
| | (m, 2H), 7.47-7.41 (m, 2H), 7.29 (s, 1H), 6.96- | requires775, |
| | 6.90 (m, 2H), 6.81 (d, J = 2.1 Hz, 1H), 6.68 (dd, J = | found: m/z = 776 |
| | 8.4, 2.1 Hz, 1H), 5.05 (dd, J = 13.0, 5.4 Hz, 1H), | [M + H]⁺ |
| | 4.37 (d, J = 12.5 Hz, 1H), 4.28 (d, J = 13.5 Hz, 1H), | |
| | 3.84 (s, 4H), 3.40-3.18 (m, 3H), 3.11 (t, J = 5.4 | |
| | Hz, 4H), 3.06-2.83 (m, 3H), 2.72 (s, 3H), 2.65- | |
| | 2.34 (m, 3H), 2.05-1.98 (m, 1H), 1.91 (t, J = 5.4 | |
| | Hz, 4H), 1.86-1.70 (m, 4H), 1.59-1.52 (m, 1H). | |
| 113 | ¹H NMR (500 MHz, DMSO-d₆) δ 11.07 (s, 1H), | LCMS: |
| | 10.88 (s, 1H), 8.21 (s, 1H), 7.90 (d, J = 8.7 Hz, 1H), | C₄₁H₄₉N₁₃O₆ |
| | 7.77-7.70 (m, 1H), 7.69-7.59 (m, 2H), 7.31 (s, | requires: 819, |
| | 1H), 6.92 (s, 1H), 6.87-6.75 (m, 2H), 5.07 (dd, J = | found: m/z = 820 |
| | 12.9, 5.4 Hz, 1H), 4.28 (d, J = 12.9 Hz, 2H), 3.67- | [M + H]⁺. |
| | 3.38 (m, 7H), 3.31-3.13 (m, 3H), 3.10-2.81 (m, | |
| | 3H), 2.73-2.56 (m, 5H), 2.24-1.96 (m, 2H), 1.80 | |
| | (q, J = 14.0, 9.8 Hz, 5H), 1.55 (d, J = 13.2 Hz, 1H). | |
| 114 | ¹H NMR (500 MHz, DMSO-d₆) δ 11.09 (s, 1H), | LCMS: |
| | 10.98 (s, 1H), 9.46 (s, 1H), 7.78-7.64 (m, 2H), | C₄₅H₅₄N₁₂O₆ |
| | 7.61 (s, 1H), 7.45 (dd, J = 9.1, 3.8 Hz, 2H), 7.37 (s, | requires: 859, |
| | 1H), 7.29 (d, J = 4.4 Hz, 2H), 6.70 (dd, J = 17.3, 8.6 | found: m/z = 858.7 |
| | Hz, 2H), 5.13-4.95 (m, 1H), 4.48-4.22 (m, 3H), | [M + H]⁺ |
| | 4.11 (d, J = 13.3 Hz, 2H), 3.99 (s, 2H), 3.01 (d, J = | |
| | 10.9 Hz, 9H), 2.73 (d, J = 4.6 Hz, 4H), 2.62 (s, 4H), | |
| | 2.11-1.93 (m, 3H), 1.82 (d, J = 11.0 Hz, 5H), 1.64- | |
| | 1.50 (m, 1H), 1.30-1.23 (m, 3H). | |
| 115 | ¹H NMR (500 MHz, DMSO-d₆) δ 11.20 (s, 1H), | LCMS: |
| | 10.99 (s, 1H), 7.98 (d, J = 9.2 Hz, 1H), 7.77 (s, 1H), | C₄₄H₅₃N₁₁O₅ |
| | 7.67 (s, 1H), 7.52 (d, J = 8.2 Hz, 2H), 7.34 (s, 1H), | requires: 816, |
| | 7.21 (dd, J = 18.5, 7.8 Hz, 3H), 6.79 (d, J = 9.1 Hz, | found: m/z = 816.9 |
| | 1H), 6.58-6.44 (m, 2H), 4.50-4.23 (m, 3H), 4.04 | [M + H]⁺ |
| | (q, J = 7.1 Hz, 6H), 3.21-2.93 (m, 9H), 2.75 (s, | |
| | 6H), 2.64 (d, J = 19.4 Hz, 7H), 2.40 (d, J = 7.9 Hz, | |
| | 4H), 2.10 (t, J = 15.1 Hz, 5H), 1.88-1.53 (m, 12H). | |
| 116 | ¹H NMR (500 MHz, DMSO-d₆) δ 11.30 (s, 1H), | LCMS: |
| | 11.00 (s, 1H), 8.02 (d, J = 8.9 Hz, 1H), 7.79 (s, 1H), | C₄₃H₅₁N₁₁O₅ |
| | 7.69 (d, J = 3.0 Hz, 1H), 7.58 (d, J = 8.1 Hz, 2H), | requires: 801, |
| | 7.36 (s, 1H), 7.28 (d, J = 7.4 Hz, 1H), 7.19 (d, J = | found: m/z = 802 |
| | 8.3 Hz, 3H), 6.65 (dd, J = 8.8, 2.3 Hz, 1H), 6.53 (s, | [M + H]⁺ |
| | 1H), 6.47 (d, J = 7.7 Hz, 2H), 4.42-4.15 (m, 4H), | |
| | 3.80 (d, J = 7.5 Hz, 2H), 3.69-3.47 (m, 4H), 3.04 | |
| | (dd, J = 61.3, 12.4 Hz, 5H), 2.82-2.57 (m, 6H), | |
| | 1.92 (d, J = 95.5 Hz, 8H), 1.25 (s, 5H). | |
| 117 | ¹H NMR (500 MHz, Acetonitrile-d₃) δ 10.99 (s, | LCMS: |
| | 1H), 8.76 (s, 1H), 7.58-7.48 (m, 4H), 7.36 (d, J = | C₄₃H₅₃N₁₁O₅ |
| | 20.8 Hz, 1H), 7.19 (d, J = 8.1 Hz, 2H), 6.64 (d, J = | requires: 803, |
| | 7.8 Hz, 2H), 5.76 (s, 1H), 5.08-4.88 (m, 2H), 4.42 | found: m/z = 804 |
| | (s, 1H), 4.35-4.11 (m, 3H), 3.69 (s, 1H), 3.59 (s, | [M + H]⁺ |
| | 1H), 3.52 (s, 0H), 3.48 (d, J = 8.2 Hz, 0H), 3.46- | |
| | 3.39 (m, 1H), 3.39-3.24 (m, 2H), 3.15-3.05 (m, | |
| | 1H), 2.99 (dt, J = 24.0, 12.3 Hz, 3H), 2.87 (s, 1H), | |
| | 2.77 (s, 3H), 2.72 (s, 0H), 2.71-2.58 (m, 2H), 2.48 | |
| | (s, 1H), 2.40 (d, J = 7.8 Hz, 2H), 1.87 (s, 2H), 1.79 | |
| | (d, J = 11.0 Hz, 4H), 1.69 (s, 8H), 1.27 (s, 2H), 1.15- | |
| | 1.04 (m, 0H), 0.84 (d, J = 6.6 Hz, 1H). | |
| 118 | ¹H NMR (500 MHz, Acetonitrile-d₃) δ 11.23 (d, J = | LCMS: |
| | 5.4 Hz, 1H), 8.80 (s, 1H), 7.84 (d, J = 2.8 Hz, 1H), | C₃₈H₄₈N₁₂O₅ |
| | 7.73-7.66 (m, 2H), 7.60 (s, 1H), 7.48-7.35 (m, | requires: 752, |
| | 2H), 7.30 (d, J = 8.5 Hz, 2H), 5.84 (t, J = 2.7 Hz, | found: m/z = 753 |
| | 2H), 5.57 (dd, J = 12.3, 5.4 Hz, 1H), 4.61 (s, 2H), | [M + H]⁺ |
| | 4.56-4.30 (m, 3H), 4.28-4.08 (m, 2H), 4.08- | |
| | 3.93 (m, 2H), 3.91 (d, J = 13.5 Hz, 2H), 3.77-3.63 | |
| | (m, 1H), 3.45-3.15 (m, 6H), 3.15-2.88 (m, 7H), | |
| | 2.63-2.49 (m, 6H), 2.14 (ddt, J = 10.5, 5.5, 2.8 Hz, | |
| | 2H), 1.89-1.74 (m, 5H), 1.74-1.57 (m, 2H), 1.51- | |
| | 1.19 (m, 3H). | |
| 119 | ¹H NMR (500 MHz, Acetonitrile-d₃) δ 11.23 (d, J = | LCMS: |
| | 6.0 Hz, 1H), 9.50 (s, 1H), 8.85 (s, 1H), 7.70 (dd, J = | C₄₃H₅₃N₁₃O₅ |
| | 8.6, 4.5 Hz, 2H), 7.61 (s, 1H), 7.57-7.48 (m, 2H), | requires: 831, |
| | 7.45 (s, 1H), 7.37 (d, J = 8.3 Hz, 1H), 7.35-7.28 | found: m/z = 832 |
| | (m, 2H), 7.07 (dd, J = 9.0, 2.0 Hz, 2H), 5.85 (s, 1H), | [M + H]⁺ |
| | 5.04 (dd, J = 12.4, 5.2 Hz, 1H), 4.61 (q, J = 8.2, 7.2 | |
| | Hz, 2H), 4.56-4.40 (m, 2H), 4.40-3.98 (m, 5H), | |
| | 3.88 (d, J = 12.8 Hz, 2H), 3.74-3.67 (m, 1H), 3.43- | |
| | 3.35 (m, 2H), 3.35-3.26 (m, 7H), 3.17 (t, J = 6.5 | |
| | Hz, 3H), 3.13-2.95 (m, 6H), 2.93-2.81 (m, 5H), | |
| | 2.60 (qd, J = 12.6, 5.5 Hz, 3H), 2.23 (dtd, J = 13.6, | |
| | 5.2, 3.5 Hz, 1H), 1.91-1.78 (m, 6H), 1.74-1.59 | |
| | (m, 2H), 1.49-1.37 (m, 2H). | |
| 120 | ¹H NMR (500 MHz, Acetonitrile-d₃) δ 11.13 (s, | LCMS: |
| | 1H), 9.75 (s, 1H), 8.80 (s, 1H), 7.85 (d, J = 2.9 Hz, | C₄₀H₅₂N₁₂O₅ |
| | 1H), 7.71-7.54 (m, 3H), 7.43 (s, 1H), 7.22 (t, J = | requires: 780, |
| | 9.5 Hz, 2H), 5.89-5.72 (m, 2H), 5.57 (dd, J = 12.2, | found: m/z = 781 |
| | 5.3 Hz, 1H), 4.37 (dd, J = 50.3, 13.4 Hz, 3H), 3.92 | [M + H]⁺ |
| | (d, J = 13.4 Hz, 3H), 3.69 (dd, J = 12.1, 8.5 Hz, 4H), | |
| | 3.58-3.16 (m, 7H), 3.16-3.03 (m, 2H), 3.03- | |
| | 2.87 (m, 9H), 2.88-2.82 (m, 2H), 2.71 (dd, J = | |
| | 15.5, 6.1 Hz, 2H), 2.59 (qd, J = 12.9, 4.9 Hz, 5H), | |
| | 2.24-2.03 (m, 10H), 1.95-1.80 (m, 8H), 1.68 (t, J = | |
| | 12.1 Hz, 2H), 1.37 (q, J = 12.6 Hz, 3H). | |
| 121 | ¹H NMR (500 MHz, Methanol-d₄) δ 7.63 (s, 1H), | LCMS: |
| | 7.60-7.49 (m, 4H), 7.20 (d, J = 8.3 Hz, 2H), 7.09 | C₄₀H₅₂N₁₂O₅ |
| | (d, J = 8.6 Hz, 2H), 5.19 (dd, J = 12.6, 5.3 Hz, 2H), | requires: 859, |
| | 4.41 (dd, J = 37.5, 13.0 Hz, 2H), 3.91 (d, J = 12.7 | found: m/z = 860 |
| | Hz, 2H), 3.84-3.70 (m, 1H), 3.54-3.40 (m, 6H), | [M + H]⁺ |
| | 3.40-3.36 (m, 3H), 3.20-3.06 (m, 4H), 3.02 (t, J = | |
| | 12.5 Hz, 2H), 2.96-2.86 (m, 2H), 2.77-2.49 (m, | |
| | 3H), 2.41 (d, J = 6.7 Hz, 2H), 2.28-2.18 (m, 2H), | |
| | 2.01-1.77 (m, 10H), 1.69 (dd, J = 27.1, 14.7 Hz, | |
| | 2H), 1.47-1.32 (m, 3H). | |
| 122 | ¹H NMR (500 MHz, CD₃CN) δ 11.09 (s, 1H), 10.21 | LCMS: |
| | (s, 1H), 8.71 (s, 1H), 7.77 (d, J = 8.7 Hz, 1H), 7.60- | C₄₅H₅₆N₁₀O₅ |
| | 7.53 (m, 3H), 7.40 (s, 1H), 7.19 (d, J = 8.3 Hz, 2H), | requires: 816, |
| | 6.86 (dd, J = 8.9, 2.5 Hz, 1H), 6.74 (d, J = 2.6 Hz, | found: m/z = 817 |
| | 1H), 5.80 (s, 1H), 5.15 (s, 1H), 4.37 (d, J = 12.7 Hz, | [M + H]⁺ |
| | 1H), 4.29 (d, J = 13.6 Hz, 1H), 3.97-3.86 (m, 3H), | |
| | 3.64 (d, J = 12.3 Hz, 2H), 3.43 (tdd, J = 21.4, 11.4, | |
| | 5.2 Hz, 4H), 3.09-2.63 (m, 12H), 2.50 (s, 1H), | |
| | 2.42-2.10 (m, 8H), 2.04-1.97 (m, 3H), 1.89- | |
| | 1.79 (m, 6H), 1.71-1.60 (m, 1H), 1.37 (q, J = 11.0, | |
| | 10.5 Hz, 2H). | |
| 123 | ¹H NMR (500 MHz, DMSO-d₆) δ 11.26 (s, 1H), | LCMS: |
| | 11.17 (s, 1H), 9.55 (s, 1H), 8.17-8.03 (m, 2H), | C₃₉H₄₅N₉O₅ |
| | 7.98 (d, J = 7.7 Hz, 1H), 7.75 (s, 1H), 7.68 (s, 1H), | requires: 719.8, |
| | 7.57 (s, 1H), 7.32 (s, 1H), 7.17 (s, 1H), 5.21 (dd, J = | found: m/z = 720.6 |
| | 13.0, 5.4 Hz, 1H), 4.57 (d, J = 4.9 Hz, 2H), 3.33- | [M + H]⁺ |
| | 3.03 (m, 8H), 2.92 (ddd, J = 18.4, 13.9, 5.4 Hz, 1H), | |
| | 2.74-2.58 (m, 1H), 2.14-2.03 (m, 1H), 1.88 (d, J = | |
| | 44.4 Hz, 3H), 1.76-1.45 (m, 11H). | |
| 124 | ¹H NMR (500 MHz, DMSO-d₆) δ 11.46 (s, 1H), | LCMS: |
| | 11.09 (s, 1H), 7.79 (s, 1H), 7.75-7.65 (m, 3H), | C₃₈H₄₃N₉O₅ |
| | 7.37 (d, J = 9.2 Hz, 2H), 7.28 (d, J = 8.7 Hz, 1H), | requires: 705.8, |
| | 5.08 (dd, J = 12.7, 5.4 Hz, 1H), 2.90 (ddd, J = 18.4, | found: m/z = 706.7 |
| | 13.9, 5.4 Hz, 1H), 2.60 (d, J = 18.7 Hz, 2H), 2.03 | [M + H]⁺ |
| | (dt, J = 11.3, 5.2 Hz, 1H), 1.83 (s, 4H), 1.65 (dq, J = | |
| | 33.5, 5.5 Hz, 10H). | |
| 125 | ¹H NMR (500 MHz, Acetonitrile-d₃) δ 11.34 (s, | LCMS: |
| | 1H), 11.16 (s, 1H), 8.89 (s, 1H), 8.57 (s, 1H), 8.51 | C₃₇H₃₈N₁₀O₅ |
| | (d, J = 2.7 Hz, 1H), 7.87 (s, 1H), 7.76 (s, 1H), 7.69 | requires: 702.8, |
| | (dd, J = 21.8, 8.3 Hz, 3H), 7.34 (d, J = 8.3 Hz, 2H), | found: m/z = 703.7 |
| | 6.98 (d, J = 2.2 Hz, 1H), 6.83 (dd, J = 8.5, 2.1 Hz, | [M + H]⁺ |
| | 1H), 6.62 (t, J = 2.1 Hz, 1H), 6.28 (s, 1H), 4.96 (dd, | |
| | J = 12.0, 5.4 Hz, 1H), 3.90-3.65 (m, 3H), 3.65- | |
| | 3.53 (m, 1H), 3.47 (q, J = 8.7 Hz, 1H), 3.31 (t, J = | |
| | 9.0 Hz, 3H), 3.12-2.83 (m, 4H), 2.83-2.62 (m, | |
| | 3H), 2.53 (s, 1H). | |
| 126 | ¹H NMR (500 MHz, DMSO-d₆) δ 11.36 (d, J = 6.8 | LCMS: |
| | Hz, 1H), 11.09 (s, 1H), 9.08 (d, J = 10.7 Hz, 1H), | C₄₁H₄₇N₉O₆ |
| | 7.78 (d, J = 7.6 Hz, 2H), 7.71 (d, J = 8.4 Hz, 1H), | requires: 761.9, |
| | 7.66 (d, J = 8.2 Hz, 2H), 7.36 (d, J = 11.7 Hz, 1H), | found: m/z = 762.8 |
| | 7.24 (d, J = 8.2 Hz, 2H), 6.97 (d, J = 2.1 Hz, 1H), | [M + H]⁺ |
| | 6.87 (dd, J = 8.6, 2.2 Hz, 1H), 5.08 (dd, J = 12.7, | |
| | 5.5 Hz, 1H), 4.32 (s, 4H), 3.98 (s, 2H), 3.83-3.65 | |
| | (m, 5H), 3.13 (t, J = 10.9 Hz, 3H), 2.88 (ddt, J = | |
| | 24.4, 16.0, 12.6 Hz, 4H), 2.63-2.55 (m, 3H), 2.31 | |
| | (dd, J = 12.3, 6.9 Hz, 1H), 2.04 (q, J = 9.5, 5.9 Hz, | |
| | 3H), 1.92 (h, J = 7.9, 5.9 Hz, 4H), 1.58 (q, J = 7.4, | |
| | 6.0 Hz, 2H). | |
| 127 | ¹H NMR (500 MHz, DMSO-d₆) δ 11.61 (s, 1H), | LCMS: |
| | 11.01 (s, 1H), 8.90 (s, 1H), 8.72 (d, J = 2.6 Hz, 1H), | C₄₀H₄₁N₁₀F₃O₄ |
| | 8.51 (d, J = 16.4 Hz, 2H), 8.10-7.96 (m, 2H), 7.72 | requires: 782.8, |
| | (d, J = 8.2 Hz, 2H), 7.37-7.18 (m, 5H), 7.00 (d, J = | found: m/z = 783.8 |
| | 2.3 Hz, 1H), 6.49 (d, J = 7.5 Hz, 1H), 4.04 (d, J = | [M + H]⁺ |
| | 12.9 Hz, 2H), 3.09 (q, J = 6.5 Hz, 5H), 3.01-2.79 | |
| | (m, 6H), 2.03 (tdd, J = 22.6, 13.7, 10.1 Hz, 6H), | |
| | 1.88 (d, J = 13.2 Hz, 3H), 1.33 (qd, J = 15.6, 15.1, | |
| | 5.7 Hz, 2H). | |
| 128 | ¹H NMR (500 MHz, DMSO-d₆) δ 11.26 (s, 1H), | LCMS: |
| | 11.08 (s, 1H), 7.84-7.75 (m, 1H), 7.75-7.59 (m, | C₃₈H₃₇N₁F₃O₅ |
| | 2H), 7.51 (d, J = 8.2 Hz, 2H), 7.40-7.31 (m, 1H), | requires: 770.8, |
| | 7.19 (d, J = 8.2 Hz, 2H), 6.98-6.75 (m, 2H), 5.06 | found: m/z = 771.8 |
| | (dd, J = 12.9, 5.4 Hz, 1H), 4.41 (d, J = 12.8 Hz, 1H), | [M + H]⁺ |
| | 4.31 (t, J = 8.0 Hz, 3H), 3.61 (dt, J = 15.9, 8.2 Hz, | |
| | 7H), 3.16 (d, J = 10.7 Hz, 6H), 3.02 (dt, J = 20.5, | |
| | 10.8 Hz, 4H), 2.92-2.85 (m, 1H), 2.64 (d, J = 28.8 | |
| | Hz, 3H), 2.40 (d, J = 7.5 Hz, 3H), 2.15 (d, J = 6.9 | |
| | Hz, 1H), 2.14-1.97 (m, 4H), 1.83 (ddd, J = 43.1, | |
| | 32.8, 8.9 Hz, 7H), 1.63 (dd, J = 30.3, 13.7 Hz, 4H). | |
| 129 | ¹H NMR (500 MHz, DMSO-d₆) δ 11.28 (s, 1H), | LCMS: |
| | 10.77 (s, 1H), 8.81 (s, 1H), 7.78 (s, 1H), 7.68 (s, | C₄₁H₅₄N₁₂O₄ |
| | 2H), 7.56 (d, J = 8.0 Hz, 2H), 7.35 (s, 1H), 7.18 (d, | requires: 778, |
| | J = 8.2 Hz, 2H), 7.05 (s, 1H), 6.73 (s, 1H), 6.52 (s, | found: m/z = 779 |
| | 1H), 5.41 (s, 1H), 4.31 (s, 2H), 4.20 (s, 1H), 4.05 (d, | [M + H]⁺ |
| | J = 12.5 Hz, 2H), 3.62 (d, J = 11.3 Hz, 4H), 3.30- | |
| | 3.20 (m, 2H), 3.06 (d, J = 14.2 Hz, 5H), 2.96 (d, J = | |
| | 12.4 Hz, 1H), 2.77 (s, 2H), 2.71 (d, J = 3.6 Hz, 3H), | |
| | 2.66 (d, J = 16.1 Hz, 2H), 2.60 (s, 0H), 2.09 (s, 1H), | |
| | 1.98 (s, 2H), 1.90 (d, J = 18.1 Hz, 2H), 1.79 (d, J = | |
| | 15.6 Hz, 6H), 1.57 (d, J = 13.8 Hz, 2H), 1.26 (d, J = | |
| | 13.6 Hz, 3H). | |
| 130 | ¹H NMR (500 MHz, DMSO-d₆) δ 11.18 (s, 1H), | LCMS: |
| | 10.84 (s, 1H), 8.69 (d, J = 8.3 Hz, 1H), 8.31 (s, 1H), | C₄₂H₅₄N₁₂O₅ |
| | 7.84 (d, J = 8.9 Hz, 1H), 7.75 (s, 1H), 7.66 (s, 1H), | requires: 806, |
| | 7.49 (d, J = 7.9 Hz, 2H), 7.40 (d, J = 9.0 Hz, 1H), | found: m/z = 807 |
| | 7.32 (s, 1H), 7.19-7.13 (m, 2H), 5.75 (s, 1H), 4.74 | [M + H]⁺ |
| | (s, 1H), 4.35 (s, 1H), 4.28 (d, J = 13.1 Hz, 1H), 3.94 | |
| | (d, J = 12.5 Hz, 2H), 3.62 (s, 1H), 3.27 (d, J = 7.7 | |
| | Hz, 2H), 3.07-2.96 (m, 1H), 2.97-2.91 (m, 3H), | |
| | 2.86 (t, J = 12.5 Hz, 2H), 2.81-2.74 (m, 1H), 2.72 | |
| | (s, 3H), 2.42 (s, 1H), 2.18 (d, J = 5.8 Hz, 3H), 2.02- | |
| | 1.93 (m, 4H), 1.85-1.78 (m, 5H), 1.77-1.71 (m, | |
| | 3H), 1.63-1.59 (m, 3H), 1.25-1.16 (m, 3H). | |
| 131 | ¹H NMR (500 MHz, Acetonitrile-d₃) δ 11.17 (s, | LCMS: |
| | 1H), 10.73 (s, 1H), 8.75 (s, 1H), 8.33-8.17 (m, | C₃₆H₄₅N₁₃O₆ |
| | 2H), 7.91 (d, J = 8.8 Hz, 1H), 7.84 (s, 1H), 7.53- | requires: 755, |
| | 7.41 (m, 2H), 7.40-7.24 (m, 2H), 5.76 (s, 1H), 4.72 | found: m/z = 756 |
| | (ddd, J = 13.1, 7.8, 5.5 Hz, 1H), 4.59 (d, J = 45.9 | [M + H]⁺ |
| | Hz, 4H), 4.31-4.08 (m, 3H), 3.93 (d, J = 13.1 Hz, | |
| | 3H), 3.84 (s, 3H), 3.77-3.70 (m, 1H), 3.14 (d, J = | |
| | 49.4 Hz, 6H), 2.89 (t, J = 12.1 Hz, 2H), 2.75 (ddd, | |
| | J = 18.5, 13.4, 5.5 Hz, 1H), 2.67 (s, 1H), 2.33-2.22 | |
| | (m, 1H), 2.13 (qd, J = 13.4, 5.2 Hz, 1H), 2.00 (d, J = | |
| | 11.5 Hz, 1H), 1.92-1.72 (m, 2H), 1.67 (t, J = 12.5 | |
| | Hz, 1H), 1.45-1.30 (m, 2H). | |
| 132 | ¹H NMR (500 MHz, Acetonitrile-d₃) δ 12.38 (s, | LCMS: |
| | 1H), 10.72 (s, 1H), 8.73 (s, 1H), 7.84 (s, 1H), 7.54 | C₃₅H₄₅N₁₃O₅ |
| | (dd, J = 9.6, 2.9 Hz, 1H), 7.47 (s, 1H), 7.45 (s, 1H), | requires: 727, |
| | 7.43 (d, J = 2.8 Hz, 1H), 7.34 (s, 1H), 7.08 (d, J = | found: m/z = 728 |
| | 9.7 Hz, 1H), 5.76 (s, 1H), 4.54 (d, J = 11.6 Hz, 2H), | [M + H]⁺ |
| | 4.18 (d, J = 14.2 Hz, 1H), 4.13 (dd, J = 12.1, 4.9 Hz, | |
| | 1H), 3.99 (d, J = 13.4 Hz, 2H), 3.90 (s, 2H), 3.83 (s, | |
| | 3H), 3.72 (dq, J = 11.0, 5.4, 4.7 Hz, 1H), 3.16 (s, | |
| | 2H), 3.13-2.99 (m, 4H), 2.77-2.64 (m, 3H), 2.31- | |
| | 2.21 (m, 3H), 1.99 (dd, J = 12.0, 5.5 Hz, 3H), 1.92- | |
| | 1.83 (m, 3H), 1.83-1.72 (m, 1H), 1.66 (dd, J = | |
| | 17.1, 8.3 Hz, 1H), 1.35 (qd, J = 12.3, 4.0 Hz, 2H). | |
| 133 | ¹H NMR (500 MHz, Acetonitrile-d₃) δ 11.14 (s, | LCMS: |
| | 1H), 9.42 (s, 1H), 8.82 (s, 1H), 8.07 (d, J = 9.1 Hz, | C₄₁H₅₉N₉O₄ |
| | 1H), 7.61 (d, J = 8.3 Hz, 2H), 7.54 (s, 1H), 7.38 (s, | requires: 731, |
| | 1H), 7.20 (d, J = 8.1 Hz, 2H), 7.17-7.11 (m, 1H), | found: m/z = 732 |
| | 7.03 (d, J = 7.4 Hz, 1H), 6.92 (d, J = 2.7 Hz, 1H), | [M + H]⁺ |
| | 6.45 (d, J = 7.5 Hz, 1H), 5.79-5.75 (m, 1H), 5.23 | |
| | (s, 1H), 3.97 (d, J = 12.9 Hz, 2H), 3.72-3.63 (m, | |
| | 6H), 3.16-3.12 (m, 1H), 3.00-2.88 (m, 5H), 2.82- | |
| | 2.73 (m, 0H), 2.14 (s, 4H), 2.13-2.06 (m, 1H), | |
| | 2.03 (d, J = 14.6 Hz, 2H), 1.71 (d, J = 7.0 Hz, 2H), | |
| | 1.64 (d, J = 7.0 Hz, 5H), 1.40 (d, J = 12.5 Hz, 2H), | |
| | 1.32 (dd, J = 10.7, 6.4 Hz, 3H). | |
| 134 | ¹H NMR (500 MHz, DMSO-d₆) δ 11.20 (s, 1H), | LCMS: |
| | 10.99 (s, 1H), 7.98 (d, J = 9.2 Hz, 1H), 7.77 (s, 1H), | C₄₁H₅₉N₉O₄ |
| | 7.67 (s, 1H), 7.52 (d, J = 8.2 Hz, 2H), 7.34 (s, 1H), | requires: 815, |
| | 7.21 (dd, J = 18.5, 7.8 Hz, 3H), 6.79 (d, J = 9.1 Hz, | found: m/z = 816 |
| | 1H), 6.58-6.44 (m, 2H), 4.50-4.23 (m, 3H), 4.04 | [M + H]⁺ |
| | (q, J = 7.1 Hz, 6H), 3.21-2.93 (m, 9H), 2.75 (s, | |
| | 6H), 2.64 (d, J = 19.4 Hz, 7H), 2.40 (d, J = 7.9 Hz, | |
| | 4H), 2.10 (t, J = 15.1 Hz, 5H), 1.88-1.53 (m, 12H). | |
| 135 | ¹H NMR (500 MHz, DMSO-d₆) δ 11.22 (s, 1H), | LCMS: |
| | 11.03 (s, 1H), 7.80-7.75 (m, 1H), 7.68 (s, 1H), | C₄₃H₅₂N₁₂O₅ |
| | 7.49 (dd, J = 8.6, 2.7 Hz, 4H), 7.37-7.33 (m, 1H), | requires 816, |
| | 7.17 (d, J = 8.2 Hz, 2H), 6.52 (d, J = 8.5 Hz, 2H), | found: m/z = 817 |
| | 5.18 (dd, J = 12.6, 5.3 Hz, 1H), 4.31 (d, J = 12.7 Hz, | [M + H]⁺ |
| | 2H), 4.03 (t, J = 7.6 Hz, 2H), 3.89-3.85 (m, 1H), | |
| | 3.60-3.54 (m, 2H), 3.45-3.37 (m, 2H), 3.30 (s, | |
| | 3H), 3.10 (t, J = 11.8 Hz, 1H), 3.05-2.82 (m, 5H), | |
| | 2.51 (p, J = 1.7 Hz, 5H), 2.34-2.25 (m, 2H), 2.17- | |
| | 2.01 (m, 3H), 2.01-1.93 (m, 2H), 1.87-1.72 (m, | |
| | 5H), 1.70-1.52 (m, 3H). | |
| 136 | ¹H NMR (500 MHz, DMSO-d₆) δ 11.20 (s, 1H), | LCMS: |
| | 11.03 (s, 1H), 7.79-7.74 (m, 1H), 7.67 (s, 1H), | C₄₃H₅₃N₁₃O₅ |
| | 7.53-7.45 (m, 4H), 7.36-7.32 (m, 1H), 7.17 (d, | requires 832, |
| | J = 8.2 Hz, 2H), 6.54-6.48 (m, 2H), 5.18 (dd, J = | found: m/z = 832 |
| | 12.7, 5.2 Hz, 1H), 4.37 (d, J = 12.4 Hz, 1H), 4.30 (d, | [M + H]⁺ |
| | J = 13.3 Hz, 1H), 4.02 (t, J = 7.6 Hz, 2H), 3.67- | |
| | 3.53 (m, 3H), 3.31 (d, J = 13.5 Hz, 8H), 3.09-2.79 | |
| | (m, 6H), 2.73 (s, 3H), 2.65-2.58 (m, 3H), 2.51- | |
| | 2.39 (m, 1H), 2.14-2.02 (m, 2H), 1.89-1.70 (m, | |
| | 5H), 1.61 (dq, J = 23.4, 12.0 Hz, 4H). | |
| 137 | ¹H NMR (500 MHz, Acetonitrile-d₃) δ 10.70 (s, | LCMS: |
| | 1H), 10.54 (s, 1H), 8.88 (s, 1H), 7.74 (s, 1H), 7.70- | C₃₉H₄₆N₁₂O₆ |
| | 7.59 (m, 1H), 7.56 (s, 1H), 7.48 (d, J = 5.3 Hz, 1H), | requires 778, |
| | 7.34 (s, 1H), 7.30 (s, 1H), 7.16 (d, J = 8.6 Hz, 1H), | found: m/z = 779 |
| | 5.83-5.69 (m, 1H), 4.93 (dd, J = 12.3, 5.4 Hz, 1H), | [M + H]⁺ |
| | 4.54-4.37 (m, 1H), 4.30 (s, 1H), 4.22 (d, J = 13.3 | |
| | Hz, 1H), 4.14 (dd, J = 11.1, 5.2 Hz, 1H), 4.09-3.97 | |
| | (m, 3H), 3.95 (d, J = 10.1 Hz, 1H), 3.83 (d, J = 9.4 | |
| | Hz, 3H), 3.47-3.28 (m, 2H), 3.25-3.12 (m, 2H), | |
| | 3.06 (dt, J = 19.7, 12.2 Hz, 2H), 2.97 (t, J = 12.6 Hz, | |
| | 2H), 2.85-2.61 (m, 3H), 2.38 (dt, J = 23.8, 7.2 Hz, | |
| | 2H), 2.19-2.02 (m, 1H), 1.91-1.73 (m, 6H), 1.66 | |
| | (d, J = 13.0 Hz, 1H), 1.47-1.24 (m, 2H). | |
| 138 | ¹H NMR (500 MHz, DMSO-d₆) δ 11.30 (s, 1H), | LCMS: |
| | 11.00 (s, 1H), 8.02 (d, J = 8.9 Hz, 1H), 7.79 (s, 1H), | C₄₃H₅₁N₁₁O₅ |
| | 7.69 (d, J = 3.0 Hz, 1H), 7.58 (d, J = 8.1 Hz, 2H), | requires 801, |
| | 7.36 (s, 1H), 7.28 (d, J = 7.4 Hz, 1H), 7.19 (d, J = | found: m/z = 802 |
| | 8.3 Hz, 3H), 6.65 (dd, J = 8.8, 2.3 Hz, 1H), 6.53 (s, | [M + H]⁺ |
| | 1H), 6.47 (d, J = 7.7 Hz, 2H), 4.42-4.15 (m, 4H), | |
| | 3.80 (d, J = 7.5 Hz, 2H), 3.69-3.47 (m, 4H), 3.04 | |
| | (dd, J = 61.3, 12.4 Hz, 5H), 2.82-2.57 (m, 6H), | |
| | 1.92 (d, J = 95.5 Hz, 8H), 1.25 (s, 5H). | |
| 139 | ¹H NMR (500 MHz, DMSO-d₆) δ 11.22 (s, 1H), | LCMS: |
| | 10.98 (s, 1H), 7.96 (d, J = 9.0 Hz, 1H), 7.77 (s, 1H), | C₄₃H₅₀N₁₀O₅ |
| | 7.67 (s, 1H), 7.51 (d, J = 8.0 Hz, 2H), 7.35 (s, 1H), | requires: 786, |
| | 7.25 (d, J = 7.8 Hz, 3H), 7.15 (d, J = 9.3 Hz, 1H), | found: m/z = 787 |
| | 6.94 (s, 1H), 6.46 (d, J = 7.5 Hz, 1H), 4.35-4.27 | [M + H]⁺ |
| | (m, 2H), 3.95 (d, J = 12.7 Hz, 2H), 3.86 (d, J = 10.1 | |
| | Hz, 1H), 3.61 (d, J = 6.7 Hz, 2H), 3.55 (t, J = 7.7 | |
| | Hz, 1H), 3.40 (q, J = 7.3 Hz, 2H), 3.04 (td, J = 23.5, | |
| | 20.0, 12.5 Hz, 4H), 2.84 (t, J = 12.6 Hz, 3H), 2.61 | |
| | (t, J = 14.6 Hz, 2H), 2.34 (d, J = 7.2 Hz, 3H), 2.28 | |
| | (q, J = 11.6, 9.9 Hz, 2H), 1.96 (dh, J = 17.4, 9.5, 8.4 | |
| | Hz, 3H), 1.79 (d, J = 13.1 Hz, 4H), 1.56 (d, J = 12.1 | |
| | Hz, 2H), 1.21 (q, J = 12.8, 12.4 Hz, 3H). | |
| 140 | ¹H NMR (500 MHz, CD₃CN) δ 11.06 (s, 1H), 8.88 | LCMS: |
| | (s, 1H), 7.63-7.52 (m, 4H), 7.40 (s, 1H), 7.28 (d, J = | C₄₁H₄₆N₁₀O₆ |
| | 8.1 Hz, 2H), 6.92 (s, 1H), 6.77 (d, J = 8.5 Hz, 1H), | requires: 774, |
| | 5.79 (s, 1H), 4.92 (q, J = 5.4 Hz, 1H), 4.38 (d, J = | found: m/z = 775 |
| | 12.8 Hz, 1H), 4.29 (d, J = 13.5 Hz, 1H), 3.95 (d, J = | [M + H]⁺ |
| | 12.4 Hz, 1H), 3.77 (s, 2H), 3.66 (s, 1H), 3.56 (d, J = | |
| | 8.9 Hz, 1H), 3.49 (d, J = 8.3 Hz, 1H), 3.40 (dt, J = | |
| | 17.0, 7.8 Hz, 3H), 3.29-3.20 (m, 2H), 3.17 (d, J = | |
| | 8.8 Hz, 1H), 3.02 (dt, J = 27.3, 12.5 Hz, 2H), 2.81- | |
| | 2.58 (m, 5H), 2.43 (s, 1H), 2.30 (d, J = 8.8 Hz, 2H), | |
| | 2.11 (m, 1H), 1.99 (d, J = 8.5 Hz, 2H), 1.86 (d, J = | |
| | 9.8 Hz, 3H), 1.81 (s, 1H), 1.78 (d, J = 10.4 Hz, 1H), | |
| | 1.66 (s, 1H). | |
| 141 | ¹H NMR (500 MHz, CD₃CN) δ 11.05 (s, 1H), 8.83 | LCMS: |
| | (s, 1H), 7.55 (d, J = 9.8 Hz, 3H), 7.48 (d, J = 8.5 Hz, | C₄₃H₅₂N₁₂O₅ |
| | 2H), 7.40 (s, 1H), 7.28 (d, J = 8.2 Hz, 2H), 7.00 (d, | requires: 816, |
| | J = 8.6 Hz, 2H), 5.79 (s, 1H), 5.00 (d, J = 12.4 Hz, | found: m/z = 817 |
| | 1H), 4.39 (d, J = 13.0 Hz, 1H), 4.29 (d, J = 13.8 Hz, | [M + H]⁺ |
| | 1H), 3.93 (d, J = 11.2 Hz, 1H), 3.82 (d, J = 12.7 Hz, | |
| | 2H), 3.68 (s, 2H), 3.61 (s, 1H), 3.42 (dt, J = 25.7, | |
| | 8.7 Hz, 2H), 3.30 (s, 3H), 3.14 (s, 2H), 3.01 (dt, J = | |
| | 26.3, 12.4 Hz, 2H), 2.83-2.69 (m, 4H), 2.58 (q, J = | |
| | 14.1 Hz, 1H), 2.41 (d, J = 6.7 Hz, 2H), 2.30 (d, J = | |
| | 8.7 Hz, 2H), 2.21 (m, 1H), 2.00 (d, J = 8.0 Hz, 3H), | |
| | 1.83 (d, J = 13.7 Hz, 4H), 1.67 (t, J = 13.1 Hz, 1H), | |
| | 1.55 (d, J = 12.7 Hz, 1H), 1.28 (q, J = 13.4, 13.0 Hz, | |
| | 2H). | |
| 142 | ¹H NMR (500 MHz, DMSO-d₆) δ 11.23 (d, J = 46.5 | LCMS: |
| | Hz, 1H), 11.02 (s, 1H), 7.76 (s, 1H), 7.66 (s, 1H), | C₄₄H₅₅N₁₃O₅ |
| | 7.50 (s, 4H), 7.33 (s, 1H), 7.17 (d, J = 8.3 Hz, 2H), | requires: 845, |
| | 6.62 (d, J = 8.3 Hz, 2H), 5.17 (dd, J = 12.7, 5.3 Hz, | found: m/z = 846 |
| | 1H), 4.45-4.18 (m, 2H), 3.61 (d, J = 10.8 Hz, 1H), | [M + H]⁺ |
| | 3.41 (d, J = 29.2 Hz, 3H), 3.16-2.92 (m, 5H), 2.88 | |
| | (ddd, J = 17.8, 13.3, 5.4 Hz, 1H), 2.73 (s, 3H), 2.61 | |
| | (d, J = 17.5 Hz, 4H), 2.48-2.40 (m, 4H), 2.36 (s, | |
| | 1H), 2.17-1.91 (m, 5H), 1.79 (dd, J = 30.2, 12.0 | |
| | Hz, 7H), 1.70-1.46 (m, 5H). | |
| 143 | ¹H NMR (500 MHz, CD₃CN) δ 11.09 (s, 1H), 8.83 | LCMS: |
| | (s, 1H), 7.58 (d, J = 8.3 Hz, 2H), 7.55 (s, 1H), 7.51 | C₄₅H₅₆N₁₂O₅ |
| | (d, J = 8.4 Hz, 2H), 7.40 (s, 1H), 7.18 (d, J = 8.1 Hz, | requires: 844, |
| | 2H), 7.04 (d, J = 8.5 Hz, 2H), 5.81 (s, 1H), 5.01 (dd, | found: m/z = 845 |
| | J = 12.4, 5.2 Hz, 1H), 4.40-4.33 (m, 1H), 4.29 (d, | [M + H]⁺ |
| | J = 13.5 Hz, 1H), 3.94 (dt, J = 10.7, 6.2 Hz, 1H), | |
| | 3.86 (d, J = 12.7 Hz, 2H), 3.66 (d, J = 12.2 Hz, 2H), | |
| | 3.42 (dq, J = 29.1, 8.3 Hz, 2H), 3.31 (s, 3H), 3.09- | |
| | 2.94 (m, 6H), 2.90-2.74 (m, 4H), 2.58 (qd, J = | |
| | 12.7, 5.6 Hz, 3H), 2.32 (dt, J = 9.5, 4.7 Hz, 2H), | |
| | 2.21 (dt, J = 13.3, 4.7 Hz, 1H), 2.14-1.97 (m, 6H), | |
| | 1.92-1.79 (m, 4H), 1.66 (d, J = 11.6 Hz, 1H), 1.41 | |
| | (qd, J = 12.2, 3.9 Hz, 2H). | |
| 144 | ¹H NMR (500 MHz, CD₃CN) δ 11.09 (s, 1H), 9.05 | LCMS: |
| | (s, 1H), 8.83 (s, 1H), 8.08 (d, J = 9.0 Hz, 1H), 7.63- | C₄₅H₅₄N₁₀O₅ |
| | 7.53 (m, 3H), 7.40 (s, 1H), 7.16 (dd, J = 20.0, 8.6 | requires: 814, |
| | Hz, 3H), 7.03 (d, J = 7.4 Hz, 1H), 6.92 (s, 1H), 6.45 | found: m/z = 815 |
| | (d, J = 7.5 Hz, 1H), 5.81 (s, 1H), 5.24 (s, 1H), 4.37 | [M + H]⁺ |
| | (d, J = 11.7 Hz, 1H), 4.29 (d, J = 13.6 Hz, 1H), 4.01- | |
| | 3.90 (m, 3H), 3.66 (d, J = 12.2 Hz, 2H), 3.42 (dq, | |
| | J = 28.9, 8.0 Hz, 2H), 3.10-2.88 (m, 8H), 2.88- | |
| | 2.50 (m, 7H), 2.32 (td, J = 7.9, 3.3 Hz, 2H), 2.20- | |
| | 1.96 (m, 7H), 1.92-1.79 (m, 6H), 1.66 (d, J = 13.0 | |
| | Hz, 1H), 1.46-1.35 (m, 2H). | |
| 145 | ¹H NMR (500 MHz, Acetonitrile-d₃) δ 10.73 (d, J = | LCMS: |
| | 22.4 Hz, 2H), 8.84 (s, 1H), 8.06 (d, J = 8.9 Hz, 1H), | C₄₀H₄₈N₁₂O₅ |
| | 7.74 (s, 1H), 7.66-7.52 (m, 1H), 7.48 (d, J = 4.8 | requires: 776, |
| | Hz, 1H), 7.35 (s, 1H), 7.12 (dd, J = 9.2, 2.4 Hz, 1H), | found: m/z = 777 |
| | 7.03 (d, J = 7.4 Hz, 1H), 6.90 (d, J = 2.5 Hz, 1H), | [M + H]⁺ |
| | 6.44 (d, J = 7.4 Hz, 1H), 5.76 (s, 1H), 5.23 (s, 1H), | |
| | 4.55 (dd, J = 52.9, 11.5 Hz, 1H), 4.43 (d, J = 14.0 | |
| | Hz, 1H), 4.31 (s, 2H), 4.23 (d, J = 13.3 Hz, 1H), | |
| | 4.14 (dd, J = 11.2, 5.0 Hz, 1H), 4.08-4.00 (m, 1H), | |
| | 3.95 (d, J = 12.6 Hz, 4H), 3.83 (d, J = 7.7 Hz, 3H), | |
| | 3.38 (dq, J = 31.6, 8.2 Hz, 2H), 3.25-3.12 (m, 2H), | |
| | 3.06 (dt, J = 22.9, 11.9 Hz, 2H), 2.88 (t, J = 12.6 Hz, | |
| | 2H), 2.82-2.61 (m, 2H), 2.43-2.28 (m, 2H), 2.18- | |
| | 2.09 (m, 1H), 1.85 (dd, J = 23.8, 14.3 Hz, 4H), | |
| | 1.66 (d, J = 12.6 Hz, 1H), 1.46-1.25 (m, 2H). | |
| 146 | ¹H NMR (500 MHz, CD₃CN) δ 11.09 (s, 1H), 9.86 | LCMS: |
| | (s, 1H), 8.88 (s, 1H), 7.64 (d, J = 8.4 Hz, 1H), 7.58 | C₄₃H₅₀N₁₀O₆ |
| | (d, J = 8.2 Hz, 2H), 7.54 (s, 1H), 7.40 (s, 1H), 7.18 | requires: 802, |
| | (d, J = 8.2 Hz, 2H), 6.94 (d, J = 2.3 Hz, 1H), 6.79 | found: m/z = 803 |
| | (dd, J = 8.6, 2.3 Hz, 1H), 5.81 (s, 1H), 4.93 (dd, J = | [M + H]⁺ |
| | 12.2, 5.4 Hz, 1H), 4.40-4.34 (m, 1H), 4.29 (d, J = | |
| | 13.6 Hz, 1H), 3.94 (tt, J = 10.3, 4.5 Hz, 1H), 3.72 | |
| | (ddd, J = 33.4, 15.6, 9.9 Hz, 3H), 3.55 (td, J = 9.5, | |
| | 8.6, 3.3 Hz, 1H), 3.48-3.34 (m, 3H), 3.29-3.18 | |
| | (m, 3H), 3.02 (dt, J = 31.3, 12.1 Hz, 4H), 2.93- | |
| | 2.63 (m, 4H), 2.32 (td, J = 8.1, 3.4 Hz, 3H), 2.16- | |
| | 1.96 (m, 6H), 1.86 (qd, J = 12.9, 6.4 Hz, 4H), 1.71- | |
| | 1.61 (m, 1H). | |
| 147 | ¹H NMR (500 MHz, Acetonitrile-d₃) δ 11.15 (s, | LCMS: |
| | 1H), 10.73 (s, 1H), 8.82 (s, 1H), 8.07 (d, J = 9.0 Hz, | C₃₉H₄₆N₁₂O₆ |
| | 1H), 7.84 (s, 1H), 7.47 (d, J = 6.3 Hz, 2H), 7.35 (s, | requires: 778, |
| | 1H), 7.13 (dd, J = 9.2, 2.4 Hz, 1H), 7.03 (d, J = 7.4 | found: m/z = 779 |
| | Hz, 1H), 6.91 (d, J = 2.4 Hz, 1H), 6.45 (d, J = 7.4 | [M + H]⁺ |
| | Hz, 1H), 5.76 (s, 1H), 5.23 (s, 1H), 4.59 (d, J = 42.9 | |
| | Hz, 3H), 4.22 (d, J = 21.2 Hz, 3H), 4.03-3.86 (m, | |
| | 3H), 3.84 (s, 3H), 3.74 (d, J = 11.5 Hz, 1H), 3.14 (d, | |
| | J = 49.3 Hz, 6H), 2.89 (t, J = 12.4 Hz, 2H), 2.83- | |
| | 2.69 (m, 2H), 2.14 (dd, J = 9.6, 4.6 Hz, 1H), 2.00 (d, | |
| | J = 13.8 Hz, 1H), 1.89 (d, J = 13.4 Hz, 1H), 1.79 | |
| | (dd, J = 18.5, 6.3 Hz, 2H), 1.67 (t, J = 12.9 Hz, 2H), | |
| | 1.44-1.29 (m, 2H). | |
| 148 | ¹H NMR (500 MHz, DMSO-d₆) δ 11.50 (s, 1H), | LCMS: |
| | 11.07 (s, 1H), 9.02 (s, 1H), 8.68 (s, 1H), 8.54 (s, | C₃₇H₃₇N₉O₅S |
| | 1H), 8.14 (d, J = 3.1 Hz, 1H), 8.09-8.03 (m, 2H), | requires: 719, |
| | 7.77 (d, J = 8.1 Hz, 2H), 7.70 (d, J = 8.2 Hz, 1H), | found: m/z = 720 |
| | 7.31 (d, J = 8.3 Hz, 2H), 6.95 (s, 1H), 6.89-6.83 | [M + H]⁺ |
| | (m, 1H), 5.06 (dd, J = 12.7, 5.4 Hz, 1H), 3.76 (dd, | |
| | J = 18.8, 9.5 Hz, 1H), 3.68 (d, J = 17.4 Hz, 2H), 3.58 | |
| | (t, J = 9.6 Hz, 2H), 3.25 (dd, J = 10.3, 8.2 Hz, 1H), | |
| | 3.15 (d, J = 13.2 Hz, 2H), 2.94-2.83 (m, 3H), 2.59 | |
| | (d, J = 16.0 Hz, 1H), 2.39 (s, 1H), 2.29 (s, 2H), 2.09 | |
| | (d, J = 13.1 Hz, 2H), 2.01 (dd, J = 12.5, 6.3 Hz, 1H), | |
| | 1.89 (dt, J = 32.7, 11.1 Hz, 3H). | |
| 149 | ¹H NMR (500 MHz, DMSO-d₆) δ 11.28 (s, 1H), | LCMS: |
| | 11.06 (s, 1H), 7.73 (d, J = 2.8 Hz, 1H), 7.66 (s, 1H), | C₃₉H₄₅N₉O₅ |
| | 7.64 (d, J = 8.4 Hz, 1H), 7.55-7.48 (m, 2H), 7.31 | requires: 719, |
| | (d, J = 2.9 Hz, 1H), 7.20 (s, 1H), 7.18 (s, 1H), 6.90 | found: m/z = 720 |
| | (d, J = 2.2 Hz, 1H), 6.82 (dd, J = 8.6, 2.2 Hz, 1H), | [M + H]⁺ |
| | 5.05 (dd, J = 12.9, 5.4 Hz, 1H), 3.67 (t, J = 5.5 Hz, | |
| | 4H), 3.56 (dd, J = 10.3, 7.2 Hz, 1H), 3.54-3.46 (m, | |
| | 1H), 3.40 (dt, J = 10.3, 7.5 Hz, 1H), 3.15 (dd, J = | |
| | 10.4, 6.8 Hz, 1H), 3.04 (d, J = 10.9 Hz, 1H), 2.97 (d, | |
| | J = 11.0 Hz, 1H), 2.88 (ddd, J = 17.4, 14.1, 5.5 Hz, | |
| | 1H), 2.67-2.57 (m, 2H), 2.59-2.51 (m, 1H), 2.50- | |
| | 2.40 (m, 1H), 2.37 (d, J = 7.6 Hz, 2H), 2.18-2.08 | |
| | (m, 1H), 2.09-1.96 (m, 3H), 1.82-1.71 (m, 3H), | |
| | 1.71-1.61 (m, 4H), 1.64-1.54 (m, 4H). | |
| 150 | ¹H NMR (500 MHz, DMSO-d₆) δ 11.33 (s, 1H), | LCMS: |
| | 11.17 (s, 1H), 9.47 (s, 1H), 8.63 (s, 1H), 8.50 (s, | C₃₇H₃₇N₉O₅S |
| | 1H), 8.29-7.93 (m, 6H), 7.76-7.58 (m, 2H), 7.12 | requires: 719.8, |
| | (s, 2H), 5.21 (dd, J = 12.9, 5.5 Hz, 1H), 4.57 (d, J = | found: m/z = 720.3 |
| | 5.2 Hz, 2H), 3.38-3.01 (m, 9H), 2.92 (ddd, J = | [M + H]⁺ |
| | 17.7, 13.9, 5.4 Hz, 1H), 2.64 (d, J = 18.1 Hz, 2H), | |
| | 2.19-2.05 (m, 1H), 1.87 (d, J = 57.7 Hz, 5H), 1.56 | |
| | (d, J = 11.3 Hz, 5H). | |
| 151 | ¹H NMR (500 MHz, DMSO-d₆) δ 11.09 (s, 1H), | LCMS: |
| | 8.65 (s, 1H), 8.52 (s, 1H), 8.14 (d, J = 3.2 Hz, 1H), | C₃₆H₃₅N₉O₅S |
| | 8.05 (d, J = 19.0 Hz, 2H), 7.68 (d, J = 8.5 Hz, 2H), | requires: 705.8, |
| | 7.35 (s, 1H), 7.27 (d, J = 8.6 Hz, 1H), 5.08 (dd, J = | found: m/z = 706.3 |
| | 12.7, 5.5 Hz, 1H), 2.99-2.77 (m, 1H), 2.62 (s, 3H), | [M + H]⁺ |
| | 2.13-1.97 (m, 1H), 1.68 (d, J = 42.6 Hz, 7H). | |
| 152 | ¹H NMR (500 MHz, Acetonitrile-d₃) δ 10.99 (s, | LCMS: |
| | 1H), 8.76 (s, 1H), 7.58-7.48 (m, 4H), 7.36 (d, J = | C₄₃H₅₃N₁₁O₅ |
| | 20.8 Hz, 1H), 7.19 (d, J = 8.1 Hz, 2H), 6.64 (d, J = | requires: 803, |
| | 7.8 Hz, 2H), 5.76 (s, 1H), 5.08-4.88 (m, 2H), 4.42 | found: m/z = 804 |
| | (s, 1H), 4.35-4.11 (m, 3H), 3.69 (s, 1H), 3.59 (s, | [M + H]⁺ |
| | 1H), 3.52 (s, 0H), 3.48 (d, J = 8.2 Hz, 0H), 3.46- | |
| | 3.39 (m, 1H), 3.39-3.24 (m, 2H), 3.15-3.05 (m, | |
| | 1H), 2.99 (dt, J = 24.0, 12.3 Hz, 3H), 2.87 (s, 1H), | |
| | 2.77 (s, 3H), 2.72 (s, 0H), 2.71-2.58 (m, 2H), 2.48 | |
| | (s, 1H), 2.40 (d, J = 7.8 Hz, 2H), 1.87 (s, 2H), 1.79 | |
| | (d, J = 11.0 Hz, 4H), 1.69 (s, 8H), 1.27 (s, 2H), 1.15- | |
| | 1.04 (m, 0H), 0.84 (d, J = 6.6 Hz, 1H). | |
| 153 | ¹H NMR (500 MHz, DMSO-d₆) δ 11.37 (d, J = 80.3 | LCMS: |
| | Hz, 2H), 11.09 (s, 1H), 8.75-8.48 (m, 2H), 8.20- | C₄₄H₅₂N₁₀O₆ |
| | 7.98 (m, 2H), 7.93-7.61 (m, 3H), 7.47-7.13 (m, | requires: 816, |
| | 2H), 5.08 (dd, J = 12.8, 5.4 Hz, 1H), 3.47 (m, 27H), | found: m/z = 814 |
| | 2.90 (ddd, J = 16.8, 13.8, 5.4 Hz, 1H), 2.10-1.93 | [M + H]⁺ |
| | (m, 1H), 1.72 (d, J = 53.4 Hz, 8H). | |
| 154 | ¹H NMR (500 MHz, Acetonitrile-d₃) δ 11.12 (s, | LCMS: |
| | 1H), 9.79 (s, 1H), 8.92 (s, 1H), 7.68-7.50 (m, 4H), | C₄₅H₅₅N₁₁O₆ |
| | 7.43 (s, 1H), 7.21 (d, J = 8.2 Hz, 2H), 7.08 (dd, J = | requires: 845, |
| | 21.8, 7.8 Hz, 2H), 6.17 (s, 1H), 5.84 (s, 1H), 4.96 | found: m/z = 846 |
| | (dd, J = 12.4, 5.3 Hz, 1H), 4.36 (dd, J = 48.4, 13.3 | [M + H]⁺. |
| | Hz, 2H), 3.68 (t, J = 14.8 Hz, 3H), 3.52 (s, 1H), | |
| | 3.46-3.22 (m, 3H), 3.16-2.89 (m, 5H), 2.88-2.62 (m, | |
| | 6H), 2.19-2.01 (m, 5H), 1.68 (t, J = 12.6 Hz, 1H), | |
| | 1.46-1.12 (m, 4H). | |
| 155 | ¹H NMR (500 MHz, Acetonitrile-d₃) δ 11.11 (s, | LCMS: |
| | 1H), 10.44 (s, 1H), 8.93 (s, 1H), 7.66-7.52 (m, | C₄₁H₄₉N₁₁O₆ |
| | 4H), 7.43 (s, 1H), 7.20 (d, J = 8.2 Hz, 2H), 7.10 (d, | requires: 791, |
| | J = 7.7 Hz, 2H), 6.47 (s, 1H), 5.83 (s, 1H), 4.97 (dd, | found: m/z = 792 |
| | J = 12.5, 5.4 Hz, 1H), 4.42 (d, J = 12.6 Hz, 1H), | [M + H]⁺. |
| | 4.31 (d, J = 13.6 Hz, 1H), 3.66 (dd, J = 31.4, 11.6 | |
| | Hz, 2H), 3.52-3.22 (m, 8H), 3.22-2.89 (m, 5H), | |
| | 2.89-2.64 (m, 9H), 2.19-2.04 (m, 6H), 1.93- | |
| | 1.56 (m, 4H). | |
| 156 | ¹H NMR (500 MHz, DMSO-d₆) δ 11.20 (s, 1H), | LCMS: |
| | 11.07 (s, 1H), 7.77 (s, 1H), 7.67 (s, 1H), 7.51 (d, J = | C₄₄H₅₆N₁₂O₅ |
| | 8.3 Hz, 2H), 7.36-7.31 (m, 1H), 7.18 (d, J = 8.2 | requires 832, |
| | Hz, 2H), 6.94 (d, J = 8.6 Hz, 1H), 6.86-6.79 (m, | found: m/z = 833 |
| | 1H), 6.65 (dd, J = 8.7, 2.2 Hz, 1H), 5.30 (dd, J = | [M + H]⁺. |
| | 12.9, 5.4 Hz, 1H), 4.34 (dd, J = 39.6, 12.9 Hz, 2H), | |
| | 3.67-3.54 (m, 4H), 3.33 (s, 2H), 3.12-2.84 (m, | |
| | 6H), 2.77-2.58 (m, 7H), 2.29-2.14 (m, 2H), 2.05- | |
| | 1.92 (m, 3H), 1.88-1.49 (m, 14H), 1.33-1.23 | |
| | (m, 3H). | |
| 157 | ¹H NMR (500 MHz, Acetonitrile-d₃) δ 8.92 (s, 1H), | LCMS: |
| | 7.67-7.52 (m, 4H), 7.22 (d, J = 8.4 Hz, 2H), 7.10 | C₄₀H₄₅N₁₁O₇ |
| | (d, J = 7.2 Hz, 1H), 7.03 (d, J = 8.5 Hz, 1H), 5.04- | requires: 791, |
| | 4.93 (m, 1H), 4.76-4.63 (m, 1H), 4.41 (d, J = 12.9 | found: m/z = 792 |
| | Hz, 1H), 4.27 (d, J = 13.4 Hz, 1H), 4.22-4.09 (m, | [M + H]⁺. |
| | 2H), 4.07-3.91 (m, 1H), 3.71 (t, J = 10.8 Hz, 1H), | |
| | 3.48-3.16 (m, 4H), 3.06 (dt, J = 33.2, 12.3 Hz, | |
| | 1H), 2.91-2.70 (m, 6H), 2.19-2.07 (m, 1H), 1.93- | |
| | 1.40 (m, 8H). | |
| 158 | ¹H NMR (500 MHz, Acetonitrile-d₃) δ 9.07 (d, J = | LCMS: |
| | 22.0 Hz, 1H), 7.75 (t, J = 7.9 Hz, 1H), 7.58-7.44 | C₄₀H₄₄N₁₀O₈ |
| | (m, 4H), 7.31 (d, J = 8.5 Hz, 1H), 7.21 (d, J = 8.2 | requires: 792, |
| | Hz, 2H), 5.21-4.92 (m, 3H), 4.59 (d, J = 13.3 Hz, | found: m/z = 793 |
| | 1H), 4.32-4.24 (m, 1H), 4.17 (d, J = 13.5 Hz, 1H), | [M + H]⁺. |
| | 3.94 (d, J = 13.6 Hz, 1H), 3.72 (tt, J = 10.0, 4.0 Hz, | |
| | 1H), 3.48-3.29 (m, 4H), 3.29-3.01 (m, 3H), 2.20- | |
| | 2.07 (m, 1H), 2.02-1.43 (m, 9H). | |
| 159 | ¹H NMR (500 MHz, Acetonitrile-d₃) δ 11.17 (s, | LCMS: |
| | 1H), 10.21 (s, 1H), 8.89 (s, 1H), 7.74-7.55 (m, | C₄₀H₄₅N₉O₇ |
| | 4H), 7.43 (s, 1H), 7.25 (d, J = 8.2 Hz, 2H), 6.98 (d, | requires: 763, |
| | J = 2.2 Hz, 1H), 6.83 (dd, J = 8.5, 2.3 Hz, 1H), | found: m/z = 764 |
| | 5.88-5.77 (m, 1H), 4.96 (dd, J = 12.1, 5.3 Hz, 1H), 4.35 | [M + H]⁺. |
| | (d, J = 13.5 Hz, 2H), 3.85-3.67 (m, 3H), 3.58 (d, J = | |
| | 8.8 Hz, 1H), 3.47 (q, J = 9.4, 8.7 Hz, 1H), 3.35- | |
| | 3.15 (m, 5H), 3.12-2.57 (m, 8H), 1.70 (td, J = | |
| | 14.5, 7.4 Hz, 2H). | |
| 160 | ¹H NMR (500 MHz, Acetonitrile-d₃) δ 11.17 (s, | LCMS: |
| | 1H), 8.89 (s, 1H), 7.66 (dd, J = 13.4, 8.3 Hz, 3H), | C₄₀H₄₇N₉O₆ |
| | 7.59 (d, J = 3.8 Hz, 1H), 7.41 (s, 1H), 7.24 (d, J = | requires: 749, |
| | 8.1 Hz, 2H), 6.98 (d, J = 2.3 Hz, 1H), 6.83 (dd, J = | found: m/z = 750 |
| | 8.6, 2.2 Hz, 1H), 5.80 (s, 1H), 4.96 (dd, J = 12.1, 5.3 | [M + H]⁺. |
| | Hz, 1H), 4.47 (d, J = 12.3 Hz, 3H), 3.88-3.65 (m, | |
| | 3H), 3.59 (t, J = 8.7 Hz, 1H), 3.52-3.18 (m, 5H), | |
| | 3.10-2.62 (m, 8H), 1.38-1.16 (m, 2H). | |
| 161 | ¹H NMR (500 MHz, Acetonitrile-d₃) δ 11.19 (s, | LCMS: |
| | 1H), 10.26 (s, 1H), 8.90 (s, 1H), 7.71-7.60 (m, | C₃₈H₄₃N₉O₆ |
| | 3H), 7.55 (s, 1H), 7.46 (s, 1H), 7.24 (d, J = 8.4 Hz, | requires: 721, |
| | 2H), 6.97 (d, J = 2.3 Hz, 1H), 6.82 (dd, J = 8.5, 2.3 | found: m/z = 722 |
| | Hz, 1H), 5.87 (s, 1H), 4.96 (dd, J = 12.2, 5.4 Hz, | [M + H]⁺. |
| | 1H), 3.86-3.63 (m, 10H), 3.58 (td, J = 9.5, 8.4, 3.3 | |
| | Hz, 1H), 3.47 (q, J = 9.2, 8.7 Hz, 1H), 3.34-3.17 | |
| | (m, 3H), 3.11-2.62 (m, 6H), 2.20-2.00 (m, 4H). | |
| 162 | ¹H NMR (500 MHz, Acetonitrile-d₃) δ 11.28 (s, | LCMS: |
| | 1H), 10.26 (s, 1H), 8.89 (s, 1H), 7.73 (d, J = 8.5 Hz, | C₃₇H₄₁N₉O₅ |
| | 2H), 7.67 (d, J = 8.4 Hz, 1H), 7.42 (s, 1H), 7.26- | requires: 691, |
| | 7.19 (m, 2H), 7.15 (s, 1H), 6.98 (d, J = 2.3 Hz, 1H), | found: m/z = 692 |
| | 6.82 (dd, J = 8.5, 2.3 Hz, 1H), 5.79 (s, 1H), 4.96 | [M + H]⁺. |
| | (dd, J = 12.1, 5.4 Hz, 1H), 4.22 (t, J = 7.5 Hz, 4H), | |
| | 3.87-3.66 (m, 4H), 3.63-3.54 (m, 1H), 3.53- | |
| | 3.17 (m, 5H), 3.11-2.64 (m, 7H), 2.49 (p, J = 7.5 | |
| | Hz, 2H). | |
| 163 | ¹H NMR (500 MHz, DMSO-d₆) δ 11.33 (s, 1H), | LCMS: |
| | 11.01 (s, 1H), 8.82 (s, 1H), 7.75 (s, 1H), 7.68 (s, | C₄₁H₄₉N₉O₄ |
| | 1H), 7.61-7.50 (m, 5H), 7.33 (s, 1H), 7.20 (dd, J = | requires: 732, |
| | 7.6, 2.3 Hz, 3H), 6.57 (d, J = 7.3 Hz, 1H), 3.87 (d, | found: m/z = 733 |
| | J = 12.6 Hz, 2H), 3.68 (t, J = 5.5 Hz, 5H), 3.66-3.60 | [M + H]⁺. |
| | (m, 2H), 3.07 (q, J = 6.0, 5.5 Hz, 3H), 2.82 (dt, J = | |
| | 23.4, 12.1 Hz, 4H), 2.67-2.58 (m, 3H), 2.38-2.35 | |
| | (m, 1H), 2.11-1.83 (m, 5H), 1.69-1.63 (m, 2H), | |
| | 1.59 (p, J = 5.8 Hz, 4H), 1.37 (q, J = 12.0 Hz, 2H). | |
| 164 | ¹H NMR (500 MHz, DMSO-d₆) δ 11.33 (s, 1H), | LCMS: |
| | 10.93 (s, 1H), 8.89 (s, 1H), 7.91 (d, J = 3.1 Hz, 1H), | C₃₇H₄₇N₉O₄ |
| | 7.75 (s, 1H), 7.68 (s, 1H), 7.58 (d, J = 8.5 Hz, 2H), | requires: 682, |
| | 7.46-7.40 (m, 1H), 7.32 (d, J = 5.2 Hz, 1H), 7.20 | found: m/z = 683 |
| | (d, J = 8.4 Hz, 2H), 6.93 (d, J = 9.3 Hz, 1H), 5.04 | [M + H]⁺. |
| | (dd, J = 10.7, 5.0 Hz, 1H), 4.18 (d, J = 13.0 Hz, 2H), | |
| | 3.68 (t, J = 5.4 Hz, 4H), 3.62 (d, J = 12.0 Hz, 2H), | |
| | 3.04 (t, J = 6.3 Hz, 4H), 2.81 (dt, J = 20.5, 12.0 Hz, | |
| | 3H), 2.64 (qt, J = 12.6, 5.9 Hz, 2H), 2.21 (dq, J = | |
| | 12.9, 4.9 Hz, 1H), 2.12 (ddt, J = 13.1, 11.1, 5.6 Hz, | |
| | 2H), 1.97 (q, J = 13.6, 12.5 Hz, 3H), 1.82 (d, J = | |
| | 13.3 Hz, 2H), 1.69-1.63 (m, 2H), 1.59 (t, J = 5.7 | |
| | Hz, 4H), 1.25 (q, J = 12.3, 11.5 Hz, 2H). | |
| 165 | ¹H NMR (500 MHz, DMSO-d₆) δ 11.28 (d, J = 7.6 | LCMS: |
| | Hz, 1H), 10.94 (s, 1H), 8.93 (s, 1H), 7.91 (d, J = 3.0 | C₄₁H₅₃N₁₁O₅ |
| | Hz, 1H), 7.78 (s, 1H), 7.68 (d, J = 2.2 Hz, 1H), 7.56 | requires: 780, |
| | (d, J = 8.0 Hz, 2H), 7.46 (d, J = 9.2 Hz, 1H), 7.35 (s, | found: m/z = 781 |
| | 1H), 7.16 (dd, J = 13.4, 8.1 Hz, 2H), 6.96 (d, J = 9.3 | [M + H]⁺. |
| | Hz, 1H), 5.05 (dd, J = 10.7, 5.0 Hz, 1H), 4.30 (d, J = | |
| | 20.5 Hz, 2H), 4.18 (d, J = 12.9 Hz, 2H), 3.62 (d, J = | |
| | 11.5 Hz, 3H), 3.34 (tt, J = 15.5, 8.8 Hz, 2H), 3.26 (t, | |
| | J = 7.8 Hz, 2H), 3.12-3.01 (m, 4H), 2.97 (t, J = | |
| | 12.4 Hz, 1H), 2.85 (t, J = 12.4 Hz, 1H), 2.77 (d, J = | |
| | 12.4 Hz, 1H), 2.71 (s, 3H), 2.65 (dt, J = 13.7, 5.3 | |
| | Hz, 2H), 2.21 (dd, J = 11.6, 6.4 Hz, 1H), 2.17-2.08 | |
| | (m, 2H), 2.03-1.87 (m, 4H), 1.82 (d, J = 12.4 Hz, | |
| | 4H), 1.76 (s, 1H), 1.57 (d, J = 12.6 Hz, 1H), 1.26 (q, | |
| | J = 12.3, 11.9 Hz, 2H). | |
| 166 | ¹H NMR (500 MHz, CD₃CN) δ 11.10 (s, 1H), 9.12 | LCMS: |
| | (s, 1H), 8.80 (s, 1H), 7.81 (d, J = 9.5 Hz, 1H), 7.59 | C₄₁H₅₄N₁₂O₄ |
| | (d, J = 8.0 Hz, 2H), 7.55 (s, 1H), 7.41 (s, 1H), 7.37 | requires: 779, |
| | (s, 1H), 7.19 (d, J = 7.9 Hz, 2H), 6.96 (d, J = 9.7 Hz, | found: m/z = 780 |
| | 1H), 5.81 (s, 1H), 4.51 (dd, J = 12.3, 5.1 Hz, 1H), | [M + H]⁺. |
| | 4.39 (d, J = 12.9 Hz, 1H), 4.29 (d, J = 13.7 Hz, 1H), | |
| | 3.71-3.62 (m, 3H), 3.52 (d, J = 11.9 Hz, 2H), 3.42- | |
| | 3.33 (m, 1H), 3.35-3.28 (m, 1H), 3.27 (dd, J = | |
| | 10.5, 6.9 Hz, 2H), 3.06 (t, J = 11.7 Hz, 1H), 2.98 | |
| | (dd, J = 15.5, 9.8 Hz, 4H), 2.82 (s, 3H), 2.75 (d, J = | |
| | 1.7 Hz, 4H), 2.75-2.65 (m, 3H), 2.27 (dd, J = 13.0, | |
| | 4.9 Hz, 1H), 2.19-2.10 (m, 1H), 2.10-2.03 (m, | |
| | 3H), 1.97-1.89 (m, 3H), 1.88 (s, 1H), 1.80 (dd, J = | |
| | 24.4, 12.9 Hz, 2H), 1.64 (d, J = 12.5 Hz, 1H), 1.43 | |
| | (d, J = 12.7 Hz, 2H). | |
| 167 | ¹H NMR (500 MHz, CD₃CN) δ 11.13 (s, 1H), 9.63 | LCMS: |
| | (s, 1H), 8.79 (s, 1H), 7.81 (d, J = 9.7 Hz, 1H), 7.61 | C₃₇H₄₈N₁₀O₃ |
| | (d, J = 8.3 Hz, 2H), 7.54 (d, J = 2.5 Hz, 1H), 7.37 (s, | requires: 681, |
| | 2H), 7.19 (d, J = 7.9 Hz, 2H), 6.95 (d, J = 9.7 Hz, | found: m/z = 682 |
| | 1H), 5.78 (s, 1H), 4.52 (dd, J = 12.5, 4.8 Hz, 1H), | [M + H]⁺. |
| | 3.72-3.62 (m, 6H), 3.52 (d, J = 11.7 Hz, 2H), 2.98 | |
| | (d, J = 12.3 Hz, 3H), 2.81 (d, J = 13.0 Hz, 2H), 2.71 | |
| | (q, J = 12.0, 10.8 Hz, 5H), 2.28 (d, J = 12.6 Hz, 1H), | |
| | 2.12 (dt, J = 25.4, 11.2 Hz, 3H), 2.02 (d, J = 14.9 | |
| | Hz, 3H), 1.92 (s, 1H), 1.70 (s, 2H), 1.64 (s, 4H), | |
| | 1.43 (d, J = 12.7 Hz, 2H). | |
| 168 | ¹H NMR (500 MHz, CD₃CN) δ 10.70 (s, 1H), 8.86 | LCMS: |
| | (s, 1H), 7.63 (d, J = 8.4 Hz, 1H), 7.46 (s, 1H), 7.38 | C₄₀H₄₅N₁₁O₆ |
| | (s, 2H), 7.35 (s, 1H), 7.30 (d, J = 2.4 Hz, 1H), 7.19- | requires: 776, |
| | 7.14 (m, 1H), 6.41 (s, 2H), 5.71 (s, 1H), 4.93 (dd, J = | found: m/z = 777 |
| | 12.2, 5.4 Hz, 1H), 4.34 (d, J = 12.8 Hz, 1H), 4.26 | [M + H]⁺. |
| | (d, J = 13.4 Hz, 1H), 4.04 (d, J = 13.1 Hz, 2H), 3.90 | |
| | (s, 1H), 3.54 (s, 2H), 3.42 (q, J = 7.8 Hz, 1H), 3.37 | |
| | (q, J = 8.4, 7.7 Hz, 1H), 2.97 (p, J = 13.0, 12.5 Hz, | |
| | 4H), 2.72 (td, J = 18.2, 11.1 Hz, 3H), 2.29 (t, J = 8.1 | |
| | Hz, 2H), 2.02-1.95 (m, 2H), 1.82 (d, J = 13.7 Hz, | |
| | 6H), 1.64 (s, 2H), 1.25 (d, J = 22.7 Hz, 4H), 0.85 (s, | |
| | 1H). | |
| 169 | ¹H NMR (500 MHz, CD₃CN) δ 10.70 (s, 1H), 8.86 | LCMS: |
| | (s, 1H), 7.63 (d, J = 8.4 Hz, 1H), 7.46 (s, 1H), 7.38 | C₄₁H₄₆N₁₀O₆ |
| | (s, 2H), 7.35 (s, 1H), 7.30 (d, J = 2.4 Hz, 1H), 7.19- | requires: 775, |
| | 7.14 (m, 1H), 6.41 (s, 2H), 5.71 (s, 1H), 4.93 (dd, J = | found: m/z = 776 |
| | 12.2, 5.4 Hz, 1H), 4.34 (d, J = 12.8 Hz, 1H), 4.26 | [M + H]⁺. |
| | (d, J = 13.4 Hz, 1H), 4.04 (d, J = 13.1 Hz, 2H), 3.90 | |
| | (s, 1H), 3.54 (s, 2H), 3.42 (q, J = 7.8 Hz, 1H), 3.37 | |
| | (q, J = 8.4, 7.7 Hz, 1H), 2.97 (p, J = 13.0, 12.5 Hz, | |
| | 4H), 2.72 (td, J = 18.2, 11.1 Hz, 3H), 2.50 (s, 0H), | |
| | 2.29 (t, J = 8.1 Hz, 2H), 2.02-1.95 (m, 2H), 1.82 | |
| | (d, J = 13.7 Hz, 6H), 1.64 (s, 1H), 1.25 (d, J = 22.7 | |
| | Hz, 4H), 0.85 (s, 1H). | |
| 170 | ¹H NMR (500 MHz, DMSO-d₆) δ 11.36 (s, 1H), | LCMS: |
| | 10.85 (s, 1H), 8.98 (s, 1H), 7.77 (s, 1H), 7.68 (s, | C₃₈H₅₀N₁₀O₄ |
| | 1H), 7.64 (d, J = 8.2 Hz, 2H), 7.58-7.40 (m, 2H), | requires: 710.9, |
| | 7.34 (s, 1H), 7.20 (d, J = 8.4 Hz, 2H), 4.47 (d, J = | found: m/z = 711.7 |
| | 13.1 Hz, 1H), 4.34 (s, 2H), 4.25 (d, J = 13.3 Hz, | [M + H]⁺ |
| | 2H), 4.04 (d, J = 12.9 Hz, 4H), 3.64 (d, J = 12.0 Hz, | |
| | 6H), 3.53-3.25 (m, 6H), 3.14-3.01 (m, 6H), 2.92- | |
| | 2.67 (m, 5H), 2.20-2.04 (m, 3H), 2.04-1.85 (m, | |
| | 7H), 1.85-1.61 (m, 4H), 1.49 (dd, J = 17.1, 8.4 Hz, | |
| | 1H), 1.31 (q, J = 11.9, 11.5 Hz, 3H). | |
| 171 | ¹H NMR (500 MHz, Acetonitrile-d₃) δ 11.14 (s, | LCMS: |
| | 1H), 9.39 (s, 1H), 8.77 (s, 1H), 7.92 (d, J = 3.1 Hz, | C₃₈H₄₉N₉O₅ |
| | 1H), 7.67 (d, J = 8.1 Hz, 2H), 7.58 (s, 1H), 7.56- | requires: 711.9, |
| | 7.47 (m, 1H), 7.41 (s, 1H), 7.24 (d, J = 8.2 Hz, 2H), | found: m/z = 712.8 |
| | 6.95 (d, J = 9.6 Hz, 1H), 5.79 (s, 1H), 4.87 (dd, J = | [M + H]⁺ |
| | 10.4, 5.0 Hz, 1H), 4.56 (d, J = 13.1 Hz, 1H), 4.21 (t, | |
| | J = 17.6 Hz, 4H), 3.68 (d, J = 12.1 Hz, 3H), 3.51 | |
| | (dd, J = 10.7, 5.1 Hz, 2H), 3.42 (dd, J = 10.6, 7.7 | |
| | Hz, 2H), 3.26-3.07 (m, 3H), 3.00 (d, J = 13.1 Hz, | |
| | 9H), 2.85 (t, J = 11.8 Hz, 4H), 2.79-2.66 (m, 5H), | |
| | 2.05 (d, J = 14.4 Hz, 4H), 1.82 (d, J = 33.6 Hz, 6H), | |
| | 1.58 (d, J = 11.5 Hz, 3H), 1.36 (td, J = 23.5, 13.1 | |
| | Hz, 5H). | |
| 172 | ¹H NMR (500 MHz, DMSO-d₆) δ 11.35 (s, 1H), | LCMS: |
| | 11.00 (s, 1H), 8.90 (s, 1H), 8.05-7.94 (m, 1H), | C₄₂H₅₁N₉O₅ |
| | 7.83-7.71 (m, 1H), 7.71-7.58 (m, 3H), 7.40- | requires: 761.9, |
| | 7.24 (m, 2H), 7.24-7.15 (m, 3H), 7.00 (d, J = 2.4 | found: m/z = 762.8 |
| | Hz, 1H), 6.48 (d, J = 7.5 Hz, 1H), 4.47 (d, J = 13.3 | [M + H]⁺ |
| | Hz, 1H), 4.25 (d, J = 13.4 Hz, 1H), 4.03 (d, J = 12.8 | |
| | Hz, 3H), 3.63 (s, 7H), 3.39-3.30 (m, 3H), 3.15- | |
| | 3.01 (m, 5H), 3.01-2.68 (m, 5H), 2.61 (d, J = 14.2 | |
| | Hz, 2H), 2.15 (s, 1H), 2.08-1.90 (m, 5H), 1.87 (d, | |
| | J = 12.8 Hz, 2H), 1.83-1.65 (m, 3H), 1.48 (t, J = | |
| | 12.5 Hz, 1H), 1.39-1.31 (m, 2H), 1.28 (dd, J = | |
| | 17.7, 6.5 Hz, 1H). | |
| 173 | ¹H NMR (500 MHz, DMSO-d₆) δ 11.35 (s, 1H), | LCMS: |
| | 10.86 (s, 1H), 9.02 (s, 1H), 8.71 (d, J = 8.2 Hz, 1H), | C₃₉H₅₀N₁₀O₅ |
| | 8.36 (d, J = 2.6 Hz, 1H), 7.88 (d, J = 8.7 Hz, 1H), | requires: 738.9, |
| | 7.76 (s, 1H), 7.73-7.55 (m, 3H), 7.46 (dd, J = 9.0, | found: m/z = 739.8 |
| | 2.6 Hz, 1H), 7.32 (d, J = 17.4 Hz, 1H), 7.20 (d, J = | [M + H]⁺ |
| | 8.1 Hz, 2H), 4.84-4.69 (m, 1H), 4.46 (d, J = 13.2 | |
| | Hz, 1H), 4.25 (d, J = 13.4 Hz, 1H), 4.01 (d, J = 12.8 | |
| | Hz, 3H), 3.45-3.24 (m, 4H), 3.22-3.02 (m, 5H), | |
| | 2.93 (t, J = 12.4 Hz, 3H), 2.81 (qd, J = 14.0, 12.4, | |
| | 4.6 Hz, 3H), 2.19 (qd, J = 15.0, 14.0, 5.3 Hz, 2H), | |
| | 2.09-1.82 (m, 8H), 1.75 (dd, J = 38.7, 21.6 Hz, | |
| | 3H), 1.61-1.42 (m, 1H), 1.34 (dt, J = 16.9, 10.8 | |
| | Hz, 3H). | |
| 174 | ¹H NMR (500 MHz, Acetonitrile-d₃) δ 11.13 (s, | LCMS: |
| | 1H), 10.49 (s, 1H), 8.89 (s, 1H), 7.67 (dd, J = 8.4, | C₄₀H₄₇N₉O₆ |
| | 3.7 Hz, 3H), 7.58 (s, 1H), 7.40 (s, 1H), 7.25 (d, J = | requires: 749.9, |
| | 8.4 Hz, 2H), 6.98 (d, J = 2.3 Hz, 1H), 6.87-6.80 | found: m/z = 750.9 |
| | (m, 1H), 5.79 (s, 1H), 4.96 (dd, J = 12.1, 5.4 Hz, | [M + H]⁺ |
| | 1H), 4.58 (d, J = 13.0 Hz, 1H), 4.24 (d, J = 13.5 Hz, | |
| | 1H), 3.90-3.64 (m, 4H), 3.64-3.41 (m, 6H), 3.34- | |
| | 3.21 (m, 4H), 3.14 (t, J = 12.7 Hz, 2H), 2.94 (d, J = | |
| | 16.1 Hz, 5H), 2.89-2.67 (m, 8H), 1.79 (s, 5H), | |
| | 1.59 (d, J = 12.4 Hz, 1H), 1.42-1.29 (m, 2H). | |
| 175 | ¹H NMR (500 MHz, DMSO-d₆) δ 11.33 (s, 1H), | LCMS: |
| | 11.08 (s, 1H), 7.93-7.81 (m, 1H), 7.74 (s, 1H), | C₄₀H₄₅N₉O₆F₂ |
| | 7.66 (d, J = 8.4 Hz, 1H), 7.58 (d, J = 8.2 Hz, 2H), | requires: 785.9, |
| | 7.46-7.34 (m, 1H), 7.21 (d, J = 8.2 Hz, 2H), 6.93 | found: m/z = 786.8 |
| | (d, J = 2.1 Hz, 1H), 6.84 (dd, J = 8.5, 2.1 Hz, 1H), | [M + H]⁺ |
| | 5.07 (dd, J = 13.0, 5.4 Hz, 1H), 4.94 (t, J = 5.3 Hz, | |
| | 1H), 4.53 (d, J = 13.6 Hz, 1H), 4.20 (d, J = 14.0 Hz, | |
| | 1H), 3.82 (dt, J = 11.0, 4.2 Hz, 1H), 3.59 (t, J = 8.8 | |
| | Hz, 1H), 3.55-3.39 (m, 6H), 3.29-3.23 (m, 1H), | |
| | 3.18 (dd, J = 9.9, 6.4 Hz, 1H), 3.07 (d, J = 10.6 Hz, | |
| | 1H), 2.99 (d, J = 10.8 Hz, 1H), 2.90 (ddd, J = 17.4, | |
| | 14.1, 5.6 Hz, 1H), 2.71-2.60 (m, 3H), 2.40 (d, J = | |
| | 7.7 Hz, 3H), 2.25-2.14 (m, 3H), 2.08-1.98 (m, | |
| | 3H), 1.86-1.60 (m, 5H). | |
| 176 | ¹H NMR (500 MHz, DMSO-d₆) δ 11.47 (d, J = 10.1 | LCMS: |
| | Hz, 1H), 11.09 (s, 1H), 9.10 (s, 1H), 7.79 (s, 1H), | C₄₀H₄₅N₉O₆ |
| | 7.71 (d, J = 8.2 Hz, 3H), 7.36 (d, J = 23.8 Hz, 2H), | requires: 747.9, |
| | 7.24 (d, J = 8.2 Hz, 2H), 6.98 (d, J = 2.1 Hz, 1H), | found: m/z = 748.8 |
| | 6.87 (dd, J = 8.5, 2.2 Hz, 1H), 5.08 (dd, J = 12.8, | [M + H]⁺ |
| | 5.5 Hz, 1H), 4.60 (dd, J = 35.6, 6.2 Hz, 4H), 3.83 (s, | |
| | 2H), 3.78 (dd, J = 10.2, 7.3 Hz, 1H), 3.70 (t, J = | |
| | 15.4 Hz, 3H), 3.65-3.53 (m, 5H), 3.15 (dd, J = | |
| | 20.9, 10.6 Hz, 2H), 2.97-2.75 (m, 4H), 2.62 (d, J = | |
| | 3.4 Hz, 2H), 2.31 (t, J = 7.3 Hz, 3H), 2.14-2.01 | |
| | (m, 3H), 2.01-1.83 (m, 4H). | |
| 177 | ¹H NMR (500 MHz, DMSO-d₆) δ 11.35 (d, J = 5.9 | LCMS: |
| | Hz, 1H), 11.00 (s, 1H), 8.88 (s, 1H), 8.00 (d, J = 9.0 | C₄₃H₅₁N₉O₅ |
| | Hz, 1H), 7.78 (d, J = 7.5 Hz, 2H), 7.65 (d, J = 8.4 | requires: 773.9, |
| | Hz, 2H), 7.35 (d, J = 14.5 Hz, 1H), 7.28 (d, J = 7.5 | found: m/z = 774.9 |
| | Hz, 1H), 7.23 (d, J = 7.7 Hz, 3H), 7.00 (s, 1H), 6.48 | [M + H]⁺ |
| | (d, J = 7.5 Hz, 1H), 4.32 (s, 4H), 4.00 (q, J = 11.5, | |
| | 10.1 Hz, 6H), 3.83-3.39 (m, 23H), 3.13-3.00 (m, | |
| | 4H), 2.93 (t, J = 12.4 Hz, 3H), 2.81 (t, J = 10.4 Hz, | |
| | 1H), 2.61 (d, J = 14.3 Hz, 2H), 2.15 (s, 1H), 2.08- | |
| | 1.82 (m, 9H), 1.58 (d, J = 9.5 Hz, 2H), 1.33 (q, J = | |
| | 12.0 Hz, 2H). | |
| 178 | ¹H NMR (500 MHz, Acetonitrile-d₃) δ 11.11 (s, | LCMS: |
| | 1H), 8.92 (s, 1H), 8.06 (s, 1H), 7.76-7.59 (m, 5H), | C₄₄H₅₂N₁₀O₆ |
| | 7.37-7.17 (m, 4H), 5.06 (dd, J = 12.4, 5.3 Hz, 5H), | requires: 804.0, |
| | 4.37 (s, 4H), 3.99 (s, 2H), 3.85 (d, J = 12.7 Hz, 2H), | found: m/z = 804.8 |
| | 3.69 (d, J = 12.4 Hz, 2H), 3.61 (t, J = 5.6 Hz, 2H), | [M + H]⁺ |
| | 3.35 (s, 3H), 3.08 (p, J = 8.1, 6.9 Hz, 6H), 2.93- | |
| | 2.72 (m, 3H), 2.61 (qd, J = 12.6, 5.5 Hz, 1H), 2.24 | |
| | (dtd, J = 13.7, 5.3, 3.6 Hz, 2H), 2.10 (dp, J = 10.6, | |
| | 5.2, 4.5 Hz, 4H), 1.70-1.53 (m, 4H). | |
| 179 | ¹H NMR (500 MHz, DMSO-d₆) δ 11.29 (s, 1H), | LCMS: |
| | 11.07 (s, 1H), 7.74 (d, J = 2.9 Hz, 1H), 7.67 (s, 1H), | C₄₀H₅₀N₁₀O₄ |
| | 7.53 (d, J = 8.2 Hz, 2H), 7.32 (d, J = 2.9 Hz, 1H), | requires 734, |
| | 7.20 (d, J = 8.2 Hz, 2H), 6.94 (d, J = 8.5 Hz, 1H), | found: m/z = 735 |
| | 6.83 (d, J = 2.1 Hz, 1H), 6.65 (dd, J = 8.7, 2.2 Hz, | [M + H]⁺. |
| | 1H), 5.30 (dd, J = 12.8, 5.4 Hz, 1H), 3.69 (t, J = 5.4 | |
| | Hz, 4H), 3.60 (d, J = 11.8 Hz, 2H), 3.32 (s, 3H), | |
| | 3.03-2.82 (m, 3H), 2.74-2.59 (m, 4H), 2.50- | |
| | 2.42 (m, 1H), 2.21 (d, J = 7.1 Hz, 2H), 2.03-1.95 | |
| | (m, 3H), 1.86-1.72 (m, 4H), 1.71-1.56 (m, 9H), | |
| | 1.33-1.22 (m, 2H). | |
| 180 | ¹H NMR (500 MHz, DMSO-d₆) δ 11.19 (s, 1H), | LCMS: |
| | 10.75 (s, 1H), 7.83-7.74 (m, 2H), 7.67 (s, 1H), | C₄₂H₅₆N₁₂O₄ |
| | 7.50 (d, J = 8.2 Hz, 2H), 7.34 (d, J = 2.9 Hz, 1H), | requires 792, |
| | 7.24-7.15 (m, 3H), 6.75 (d, J = 92 Hz, 1H), 4.67 | found: m/z = 793 |
| | (dd, J = 12.5, 4.8 Hz, 1H), 4.38 (d, J = 12.3 Hz, 1H), | [M + H]⁺. |
| | 4.30 (d, J = 13.4 Hz, 1H), 4.08 (d, J = 12.5 Hz, 2H), | |
| | 3.67-3.59 (m, 1H), 3.31-3.24 (m, 3H), 3.10- | |
| | 2.90 (m, 4H), 2.85-2.60 (m, 11H), 2.30-2.22 (m, | |
| | 1H), 2.18 (d, J = 6.9 Hz, 2H), 1.98 (t, J = 11.4 Hz, | |
| | 2H), 1.93-1.50 (m, 13H), 1.19-1.08 (m, 2H). | |
| 181 | ¹H NMR (500 MHz, DMSO-d₆) δ 11.29 (s, 1H), | LCMS: |
| | 10.75 (s, 1H), 7.81 (d, J = 3.2 Hz, 1H), 7.74 (d, J = | C₃₈H₅₀N₁₀O₃ |
| | 2.8 Hz, 1H), 7.67 (s, 1H), 7.53 (d, J = 8.2 Hz, 2H), | requires 694, |
| | 7.32 (d, J = 2.8 Hz, 1H), 7.24-7.17 (m, 3H), 6.74 | found: m/z = 695 |
| | (d, J = 9.1 Hz, 1H), 4.67 (dd, J = 12.6, 4.9 Hz, 1H), | [M + H]⁺. |
| | 4.08 (d, J = 12.6 Hz, 2H), 3.72-3.66 (m, 4H), 2.95 | |
| | (d, J = 10.9 Hz, 2H), 2.80 (ddd, J = 18.1, 13.5, 5.4 | |
| | Hz, 1H), 2.72-2.61 (m, 6H), 2.26 (qd, J = 12.7, 4.3 | |
| | Hz, 1H), 2.18 (d, J = 7.0 Hz, 2H), 2.02-1.93 (m, | |
| | 2H), 1.92-1.86 (m, 1H), 1.81-1.71 (m, 5H), 1.71- | |
| | 1.57 (m, 9H), 1.13 (dt, J = 12.6, 9.3 Hz, 2H). | |
| 182 | ¹H NMR (500 MHz, DMSO-d₆) δ 11.29 (s, 1H), | LCMS: |
| | 10.85 (s, 1H), 8.74-8.68 (m, 1H), 8.32 (s, 1H), | C₃₈H₄₈N₁₀O₄ |
| | 7.85 (d, J = 9.3 Hz, 1H), 7.74 (s, 1H), 7.68 (s, 1H), | requires 708, |
| | 7.53 (d, J = 8.1 Hz, 2H), 7.42 (d, J = 8.9 Hz, 1H), | found: m/z = 709 |
| | 7.32 (s, 1H), 7.20 (d, J = 8.1 Hz, 2H), 4.75 (s, 1H), | |
| | 3.96 (d, J = 12.7 Hz, 2H), 3.69 (s, 3H), 3.21-3.16 | |
| | (m, 1H), 2.96 (d, J = 10.6 Hz, 2H), 2.88 (t, J = 12.6 | |
| | Hz, 2H), 2.79 (d, J = 15.1 Hz, 1H), 2.19 (s, 3H), | |
| | 2.03-1.95 (m, 3H), 1.83 (d, J = 12.9 Hz, 3H), 1.76 | |
| | (d, J = 12.4 Hz, 3H), 1.67 (s, 3H), 1.61 (s, 5H), 1.27- | |
| | 1.18 (m, 3H). | |
| 183 | ¹H NMR (500 MHz, Acetonitrile-d₃) δ 11.00 (s, | LCMS: |
| | 1H), 9.26 (s, 1H), 7.74 (dd, J = 15.7, 8.0 Hz, 1H), | C₆₄H₉₃N₁₃O₁₁ |
| | 7.64-7.37 (m, 6H), 7.27 (d, J = 7.5 Hz, 1H), 7.22- | requires 1219, |
| | 7.02 (m, 6H), 6.02 (s, 1H), 5.12-4.95 (m, 1H), 4.66 | found: m/z = 1220 |
| | (d, J = 13.5 Hz, 1H), 4.57-4.35 (m, 5H), 4.29 (d, | [M + H]⁺. |
| | J = 13.7 Hz, 1H), 4.04 (d, J = 13.6 Hz, 1H), 3.99- | |
| | 3.85 (m, 2H), 3.72 (dt, J = 23.0, 6.3 Hz, 5H), 3.44- | |
| | 3.23 (m, 4H), 3.21-2.90 (m, 3H), 2.71-2.49 (m, | |
| | 6H), 1.92-1.45 (m, 9H), 1.44-1.35 (m, 3H), 1.31- | |
| | 0.95 (m, 6H).⁻ | |
| 184 | ¹H NMR (500 MHz, Acetonitrile-d₃) δ 8.92 (s, 1H), | LCMS: |
| | 7.67-7.52 (m, 4H), 7.22 (d, J = 8.4 Hz, 2H), 7.10 | C₄₀H₄₅N₁₁O₇ |
| | (d, J = 7.2 Hz, 1H), 7.03 (d, J = 8.5 Hz, 1H), 5.04- | requires: 791, |
| | 4.93 (m, 1H), 4.76-4.63 (m, 1H), 4.41 (d, J = 12.9 | found: m/z = 792 |
| | Hz, 1H), 4.27 (d, J = 13.4 Hz, 1H), 4.22-4.09 (m, | [M + H]⁺. |
| | 2H), 4.07-3.91 (m, 1H), 3.71 (t, J = 10.8 Hz, 1H), | |
| | 3.48-3.16 (m, 4H), 3.06 (dt, J = 33.2, 12.3 Hz, | |
| | 1H), 2.91-2.70 (m, 6H), 2.19-2.07 (m, 1H), 1.93- | |
| | 1.40 (m, 8H). | |
| 185 | ¹H NMR (500 MHz, DMSO-d₆) δ 11.29 (s, 1H), | LCMS |
| | 10.67 (s, 1H), 7.95 (d, J = 2.5 Hz, 1H), 7.74 (d, J = | C₃₈H₄₉N₉O₃ |
| | 2.9 Hz, 1H), 7.67 (s, 1H), 7.53 (d, J = 8.3 Hz, 2H), | requires: 679, |
| | 7.38 (dd, J = 8.7, 2.5 Hz, 1H), 7.32 (d, J = 2.8 Hz, | found: 680 |
| | 1H), 7.20 (d, J = 8.3 Hz, 2H), 6.76 (d, J = 8.8 Hz, | [M + H]⁺. |
| | 1H), 4.23 (d, J = 12.8 Hz, 2H), 3.69 (t, J = 5.4 Hz, | |
| | 4H), 3.06-2.96 (m, 1H), 2.98-2.92 (m, 2H), 2.78- | |
| | 2.67 (m, 3H), 2.60-2.32 (m, 4H), 2.17 (d, J = 6.6 | |
| | Hz, 2H), 2.02-1.93 (m, 2H), 1.82-1.44 (m, 15H), | |
| | 1.14-1.05 (m, 2H). | |
| 186 | ¹H NMR (500 MHz, Acetonitrile-d₃) δ 11.01 (s, | LCMS: |
| | 1H), 9.17 (d, J = 3.4 Hz, 1H), 7.57 (s, 1H), 7.51 (dd, | C₅₉H₇₉FN₁₂O₈S |
| | J = 14.1, 8.1 Hz, 4H), 7.28 (d, J = 9.5 Hz, 1H), 7.16 | requires 1134, |
| | (d, J = 8.2 Hz, 2H), 7.13-7.05 (m, 1H), 7.01 (t, J = | found: m/z = 1135 |
| | 6.9 Hz, 2H), 5.88 (d, J = 10.3 Hz, 1H), 4.66 (d, J = | [M + H]⁺. |
| | 9.6 Hz, 2H), 4.55 (t, J = 8.1 Hz, 1H), 4.51-4.40 | |
| | (m, 3H), 4.38-4.22 (m, 2H), 4.16-3.97 (m, 3H), | |
| | 3.82-3.67 (m, 3H), 3.50-3.07 (m, 3H), 2.68- | |
| | 2.31 (m, 5H), 2.12 (d, J = 10.1 Hz, 2H), 1.93-1.77 | |
| | (m, 6H), 1.64 (dt, J = 48.0, 7.5 Hz, 3H), 1.41-1.16 | |
| | (m, 5H), 0.99 (s, 8H). | |
| 187 | ¹H NMR (500 MHz, Acetonitrile-d₃) δ 11.01 (s, | LCMS: |
| | 1H), 9.17 (d, J = 3.4 Hz, 1H), 7.57 (s, 1H), 7.51 (dd, | C₅₇H₇₅FN₁₂O₈S |
| | J = 14.1, 8.1 Hz, 4H), 7.28 (d, J = 9.5 Hz, 1H), 7.16 | requires 1106, |
| | (d, J = 8.2 Hz, 2H), 7.13-7.05 (m, 1H), 7.01 (t, J = | found: m/z = 1107 |
| | 6.9 Hz, 2H), 5.88 (d, J = 10.3 Hz, 1H), 4.66 (d, J = | [M + H]⁺. |
| | 9.6 Hz, 2H), 4.55 (t, J = 8.1 Hz, 1H), 4.51-4.40 | |
| | (m, 3H), 4.38-4.22 (m, 2H), 4.16-3.97 (m, 3H), | |
| | 3.82-3.67 (m, 3H), 3.50-3.07 (m, 3H), 2.68- | |
| | 2.31 (m, 5H), 2.12 (d, J = 10.1 Hz, 2H), 1.93-1.77 | |
| | (m, 6H), 1.64 (dt, J = 48.0, 7.5 Hz, 3H), 1.41-1.16 | |
| | (m, 5H), 0.99 (s, 8H). | |
| 188 | ¹H NMR (500 MHz, Acetonitrile-d₃) δ 11.02 (s, | LCMS: |
| | 1H), 8.84 (s, 1H), 7.56 (d, J = 7.0 Hz, 3H), 7.49- | C₅₇H₇₈N₁₂O₇S |
| | 7.31 (m, 6H), 7.19 (d, J = 8.0 Hz, 2H), 6.67 (d, J = | requires: 1075, |
| | 9.1 Hz, 1H), 5.85 (s, 1H), 4.97 (q, J = 7.2 Hz, 1H), | found: m/z = 1076 |
| | 4.64 (d, J = 13.4 Hz, 1H), 4.57 (d, J = 9.0 Hz, 1H), | [M + H]⁺. |
| | 4.55-4.35 (m, 3H), 4.27 (d, J = 13.7 Hz, 1H), 4.02 | |
| | (d, J = 14.0 Hz, 1H), 3.84 (d, J = 11.1 Hz, 1H), | |
| | 3.76-3.61 (m, 1H), 3.45-3.24 (m, 4H), 2.77 (s, 4H), | |
| | 2.62 (t, J = 12.7 Hz, 1H), 2.49 (s, 3H), 2.38 (s, 2H), | |
| | 2.23 (td, J = 7.1, 3.1 Hz, 2H), 2.17-2.07 (m, 1H), | |
| | 2.03-1.93 (m, 27H), 1.93-1.77 (m, 2H), 1.73- | |
| | 1.41 (m, 11H), 1.34 (d, J = 8.4 Hz, 7H), 1.01 (s, | |
| | 10H). | |
| 189 | ¹H NMR (500 MHz, Acetonitrile-d₃) δ 11.00 (s, | LCMS: |
| | 1H), 9.07 (s, 1H), 7.58-7.49 (m, 3H), 7.44 (s, 5H), | C₅₅H₇₄N₁₂O₇S |
| | 7.28 (s, 1H), 7.23-7.14 (m, 2H), 6.77 (d, J = 8.9 | requires 1046, |
| | Hz, 1H), 5.92 (s, 1H), 4.97 (p, J = 7.1 Hz, 1H), 4.65 | found: m/z = 1047 |
| | (d, J = 13.3 Hz, 1H), 4.58 (d, J = 8.9 Hz, 1H), 4.55- | [M + H]⁺. |
| | 4.35 (m, 3H), 4.26 (d, J = 13.5 Hz, 1H), 4.02 (d, J = | |
| | 13.6 Hz, 1H), 3.86 (d, J = 11.1 Hz, 1H), 3.78-3.62 | |
| | (m, 2H), 3.06-2.91 (m, 1H), 2.77 (s, 4H), 2.64 (t, | |
| | J = 12.8 Hz, 1H), 2.50 (s, 3H), 2.39 (d, J = 7.5 Hz, | |
| | 2H), 2.25 (tp, J = 13.1, 6.6, 6.1 Hz, 2H), 1.99 (s, | |
| | 13H), 1.94-1.73 (m, 4H), 1.71-1.25 (m, 11H), | |
| | 1.01 (s, 10H). | |
| 190 | ¹H NMR (500 MHz, DMSO-d₆) δ 11.19 (s, 1H), | LCMS: |
| | 10.67 (s, 1H), 7.95 (d, J = 2.4 Hz, 1H), 7.76 (d, J = | C₄₂H₅₅N₁₁O₄ |
| | 2.9 Hz, 1H), 7.67 (s, 1H), 7.50 (d, J = 8.3 Hz, 2H), | requires: 777, |
| | 7.41-7.31 (m, 2H), 7.17 (d, J = 8.3 Hz, 2H), 6.77 | found: m/z = 778 |
| | (d, J = 8.7 Hz, 1H), 4.38 (d, J = 12.2 Hz, 1H), 4.33- | [M + H]⁺. |
| | 4.20 (m, 3H), 3.67-3.53 (m, 1H), 3.32-3.23 (m, | |
| | 3H), 3.10-2.89 (m, 6H), 2.81-2.62 (m, 6H), 2.51 | |
| | (p, J = 1.8 Hz, 6H), 2.17 (d, J = 6.7 Hz, 2H), 2.02- | |
| | 1.94 (m, 2H), 1.89-1.46 (m, 14H), 1.27-1.19 (m, | |
| | 7H), 1.15-1.05 (m, 2H). | |
| 191 | ¹H NMR (500 MHz, Acetonitrile-d₃) δ 11.13 (s, | LCMS: |
| | 1H), 9.92 (s, 1H), 8.73 (s, 1H), 7.61 (d, J = 8.4 Hz, | C₃₆H₄₆N₁₀O₅ |
| | 2H), 7.54 (s, 1H), 7.39 (d, J = 9.1 Hz, 2H), 7.20 (d, | requires: 698, |
| | J = 8.4 Hz, 2H), 7.04 (d, J = 8.3 Hz, 1H), 5.76 (s, | found: m/z = 699 |
| | 1H), 4.71 (q, J = 8.5 Hz, 1H), 4.17 (d, J = 13.1 Hz, | [M + H]⁺. |
| | 2H), 3.72-3.62 (m, 7H), 3.07 (t, J = 12.8 Hz, 2H), | |
| | 2.96 (q, J = 10.8, 8.5 Hz, 4H), 2.76-2.60 (m, 2H), | |
| | 2.19-2.10 (m, 5H), 2.01 (d, J = 14.0 Hz, 2H), 1.70 | |
| | (q, J = 6.0, 5.4 Hz, 2H), 1.64 (p, J = 5.8 Hz, 5H), | |
| | 1.35 (q, J = 12.0 Hz, 2H). | |
| 192 | ¹H NMR (500 MHz, Acetonitrile-d₃) δ 11.10 (s, | LCMS: |
| | 1H), 9.54 (s, 1H), 8.74 (s, 1H), 7.59 (d, J = 8.4 Hz, | C₄₀H₅₂N₁₂O₆ |
| | 2H), 7.55 (s, 1H), 7.40 (s, 2H), 7.19 (d, J = 8.4 Hz, | requires: 796, |
| | 2H), 7.04 (d, J = 8.1 Hz, 1H), 5.80 (s, 1H), 4.71 (q, | found: m/z = 797 |
| | J = 8.5 Hz, 1H), 4.39 (d, J = 13.1 Hz, 1H), 4.29 (d, | [M + H]⁺. |
| | J = 13.7 Hz, 1H), 4.18 (d, J = 13.5 Hz, 2H), 3.70- | |
| | 3.63 (m, 4H), 3.42-3.23 (m, 4H), 3.15-3.01 (m, | |
| | 4H), 3.02-2.93 (m, 6H), 2.86-2.78 (m, 1H), 2.75 | |
| | (s, 3H), 2.72-2.59 (m, 2H), 2.20-2.00 (m, 6H), | |
| | 1.87-1.75 (m, 2H), 1.32 (dd, J = 28.8, 16.0 Hz, | |
| | 3H). | |
| 193 | ¹H NMR (500 MHz, Acetonitrile-d₃) δ 11.13 (s, | LCMS: |
| | 1H), 10.41 (s, 1H), 8.89 (s, 1H), 7.67 (dd, J = 8.4, | C₄₀H₄₇N₉O₆ |
| | 3.7 Hz, 4H), 7.58 (s, 1H), 7.50-7.36 (m, 1H), 7.25 | requires: 749, |
| | (d, J = 8.2 Hz, 2H), 6.98 (d, J = 2.3 Hz, 1H), 6.82 | found: m/z = 750 |
| | (dd, J = 8.6, 2.3 Hz, 1H), 5.79 (s, 1H), 4.96 (dd, J = | [M + H]⁺. |
| | 12.1, 5.4 Hz, 1H), 4.58 (d, J = 13.4 Hz, 1H), 4.24 (d, | |
| | J = 13.7 Hz, 1H), 3.85-3.65 (m, 4H), 3.58 (d, J = | |
| | 8.1 Hz, 2H), 3.54-3.38 (m, 4H), 3.30 (t, J = 9.1 | |
| | Hz, 2H), 3.14 (t, J = 12.0 Hz, 2H), 2.98 (d, J = 41.7 | |
| | Hz, 5H), 2.90-2.68 (m, 8H), 1.80 (d, J = 19.1 Hz, | |
| | 5H), 1.59 (d, J = 11.7 Hz, 2H), 1.48-1.27 (m, 2H). | |

### Example 66: BTK Degradation Assay

Cell Culture

TMD8 cells were obtained from Tokyo Medical and Dental University and were grown in alpha-MEM (Fisher 12571063) supplemented with 1000 heat-inactivated FBS (Coming Premium Fetal Bovine Serum from Fisher, MT35015CV).

Cellular BTK HTRF Assay

Compounds of the present invention were added to 50,000 TMD8 cells in round-bottom 96 well plates with a final DMSO concentration of <0.2% and were incubated at 37° C. 5% CO₂ for four hours. BTK levels were determined using Cisbio Total-BTK HTRF (Homologous Time-Resolved Fluorescence) kit (63ADK064PEG) according to manufacturer's protocol. Briefly, cells were incubated in 1× supplied lysis buffer for 30 minutes. In an opaque white low volume 96 well plate (Cisbio, 66PL96005), cell lysate was combined with two different specific BTK antibodies, one conjugated with Eu³⁺-Cryptate FRET donor and one conjugated with d2 FRET acceptor. Assay controls include wells containing cell lysate with only the Eu³⁺-Cryptate FRET donor antibody and wells containing both HTRF antibodies and lysis buffer without cells or control lysate provided by Cisbio. HTRF ratio was calculated as (acceptor signal at 665 nm/donor signal at 620 nm)×10⁴. Background HTRF levels were determined from the control well containing the donor, but no acceptor, antibody. Background HTRF levels were subtracted from all samples. Readouts were reported as HTRF levels relative to HTRF levels of DMSO-treated cells. Four-parameter non-linear regressions were performed in GraphPad Prism 7.02 to obtain DC₅₀ values. DC₅₀ values are provided in Table 3, wherein A<5.0 nM, 5.0 nM≤B≤15 nM, and 15 nM<C.

TABLE 3
BTK degradation activity.

| | Cellular |
|---|---|
| Compound | BTK HTRF |
| No. | TMD8: DC₅₀ |
| 1 | — |
| 2 | C |
| 3 | C |
| 4 | C |
| 5 | C |
| 6 | C |
| 7 | C |
| 8 | B |
| 9 | C |
| 10 | B |
| 11 | A |
| 12 | B |
| 13 | B |
| 14 | C |
| 15 | A |
| 16 | B |
| 17 | A |
| 18 | B |
| 19 | B |
| 20 | A |
| 21 | B |
| 22 | B |
| 23 | B |
| 24 | B |
| 25 | B |
| 26 | B |
| 27 | B |
| 28 | B |
| 29 | A |
| 30 | B |
| 31 | A |
| 32 | A |
| 33 | A |
| 34 | A |
| 35 | A |
| 36 | B |
| 37 | A |
| 38 | A |
| 39 | A |
| 40 | A |
| 41 | A |
| 42 | A |
| 43 | A |
| 44 | A |
| 45 | A |
| 46 | A |
| 47 | A |
| 48 | A |
| 49 | A |
| 50 | A |
| 51 | A |
| 52 | A |
| 53 | A |
| 54 | A |
| 55 | A |
| 56 | A |
| 57 | A |
| 58 | B |
| 59 | A |
| 60 | A |
| 61 | A |
| 62 | C |
| 63 | C |
| 64 | C |
| 65 | C |
| 66 | A |
| 67 | C |
| 68 | — |
| 69 | A |
| 70 | A |
| 71 | A |
| 72 | A |
| 73 | A |
| 74 | A |
| 75 | A |
| 76 | B |
| 77 | A |
| 78 | A |
| 79 | C |
| 80 | A |
| 81 | C |
| 82 | A |
| 83 | A |
| 84 | C |
| 85 | A |
| 86 | A |
| 87 | A |
| 88 | C |
| 89 | B |
| 90 | B |
| 91 | A |
| 92 | B |
| 93 | A |
| 94 | A |
| 95 | B |
| 96 | C |
| 97 | C |
| 98 | A |
| 99 | A |
| 100 | C |
| 101 | A |
| 102 | A |
| 103 | A |
| 104 | B |
| 105 | C |
| 106 | B |
| 107 | C |
| 108 | B |
| 109 | A |
| 110 | A |
| 111 | B |
| 112 | A |
| 113 | A |
| 114 | A |
| 115 | A |
| 116 | A |
| 117 | A |
| 118 | A |
| 119 | A |
| 120 | A |
| 121 | A |
| 122 | A |
| 123 | B |
| 124 | C |
| 125 | A |
| 126 | A |
| 127 | C |
| 128 | C |
| 129 | A |
| 130 | A |
| 131 | A |
| 132 | A |
| 133 | A |
| 134 | A |
| 135 | A |
| 136 | A |
| 137 | A |
| 138 | A |
| 139 | A |
| 140 | A |
| 141 | A |
| 142 | A |
| 143 | A |
| 144 | A |
| 145 | A |
| 146 | A |
| 147 | A |
| 148 | A |
| 149 | A |
| 150 | B |
| 151 | C |
| 152 | A |
| 153 | A |
| 154 | A |
| 155 | A |
| 156 | A |
| 157 | B |
| 158 | B |
| 159 | B |
| 160 | A |
| 161 | B |
| 162 | A |
| 163 | A |
| 164 | B |
| 165 | B |
| 166 | A |
| 167 | C |
| 168 | A |
| 169 | A |
| 170 | A |
| 171 | A |
| 172 | A |
| 173 | A |
| 174 | A |
| 175 | A |
| 176 | A |
| 177 | A |
| 178 | B |
| 179 | A |
| 180 | C |
| 181 | B |
| 182 | A |
| 183 | C |
| 184 | B |
| 185 | C |
| 186 | C |
| 187 | C |
| 188 | C |
| 189 | C |
| 190 | B |
| 191 | B |
| 192 | A |
| 193 | A |

### Example 67: Aiolos Degradation Assay

Flow Cytometry Assays.

Frozen human peripheral blood mononuclear cells (PBMCs) were thawed and treated with DMSO or compound for 24 hours and then fixed and permeabilized using a Foxp3/Transcription Factor Fixation/Permeabilization Kit (eBioscience, 00-5523). Cells were stained with fluorophore-conjugated antibodies against CD20 (Biolegend 302330), CD3 (BD Pharmingen 552127), and Aiolos (Biolegend 371106). An additional set of DMSO-treated PBMCs was stained for CD20, CD3, and an AlexaFluor 647-conjugated mouse IgGI isotype control antibody (Biolegend 400136). Stained cells were analyzed using an Attune NxT Acoustic Focusing Flow Cytometer (Thermo-Fisher A29004), and data was analyzed using FlowJo (v10.5.3) and GraphPad Prism (v7.00) software. Single lymphocytes were gated for B cells (CD20+CD3−) and T cells (CD3+CD20−), and the geometric mean fluorescence intensity (MFI) of Aiolos was calculated for each population. The MFI of the isotype control was calculated for each population and used to quantify background staining. Percent Aiolos degradation was calculated for each compound-treated sample using the following equation:

$$\% degradation = 100 \times \frac{( Sample MFI - Isotope MFI )}{( DMSO MFI - Isotope MFI}$$

Four-parameter non-linear regressions were performed in GraphPad Prism 7.02 to obtain DC₅₀ values. Aiolos T Cell DC₅₀ values are provided in Table 4, wherein A<10.0 nM, 10.0 nM≤B≤1000 nM, and 1000 nM<C.

TABLE 4
Aiolos degradation activity.

| Compound | Aiolos T Cell |
|---|---|
| No. | DC₅₀ (nM) |
| 14 | C |
| 17 | B |
| 25 | A |
| 29 | C |
| 30 | A |
| 31 | B |
| 34 | B |
| 37 | B |
| 38 | B |
| 51 | B |
| 53 | B |
| 61 | C |
| 64 | B |
| 75 | C |
| 78 | B |
| 92 | A |
| 99 | C |
| 123 | C |
| 155 | B |
| 156 | A |
| 157 | C |
| 166 | C |

### Example 68: Mouse BTK Degradation Assay with Oral Dosing

A method of determining the pharmacodynamic profile of compounds of the present invention (experimental compound) was performed by dosing either CD-1 or BALB/c mice with the compound. The experimental compound was prepared in a suitable formulation and was administered via oral gavage (PO) at a suitable dose level and frequency as informed by prior pharmacokinetic and tolerability studies. Following administration of the experimental compound, BTK levels in blood or splenocytes are measured using flow cytometry or HTRF. For assessment of BTK levels via flow cytometry, either whole blood or spleen were first treated with ACK RBC lysis buffer to facilitate lysing of red blood cells. Remaining cells were then stained with fluorophore-conjugated antibodies against CD45, TCR beta and CD45R (B220). Cell pellets were washed with 1× PBS and fixed and permeabilized for 24 hrs with Foxp3/Transcription Factor Fixation/Permeabilization Kit. Cells were then stained intracellularly with unconjugated BTK antibody and detected with a fluorophore-conjugated secondary antibody. Stained cells were run on an Attune NxT Acoustic Focusing Flow Cytometer (Thermo-Fisher A29004), and data was analyzed using FlowJo (v10.5.3) and GraphPad Prism (v7.00) software. Lymphocytes were gated for B cells defined as CD45+ TCR beta− B220+ and T cells as CD45+ TCR beta+ B220−. The BTK geometric mean fluorescence intensity (MFI) was calculated for B and T cells. Percent BTK degradation for each experimental compound treated sample was calculated using the equation described below:

$$\% degradation = 100 \times \frac{( \begin{matrix}Treated Smpl B Cell BTK MFI - \\ Treated Smpl T cell BTK MFI\end{matrix} )}{( Veh . B Cell BTK MFI - Veh . T Cell BTK MFI )}$$

Experimental compounds, i.e., compounds of the present invention, which demonstrated significant BTK degradation upon oral dosage are summarized in Table 5.

TABLE 5
Oral bioavailability in mouse model.

| Compound | |
|---|---|
| No. | Oral Bioavailability |
| 44 | Yes |
| 70 | Yes |
| 71 | Yes |
| 72 | Yes |
| 73 | Yes |
| 74 | Yes |
| 131 | Yes |
| 150 | Yes |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1-106. (canceled)

107. A compound of Formula (X): 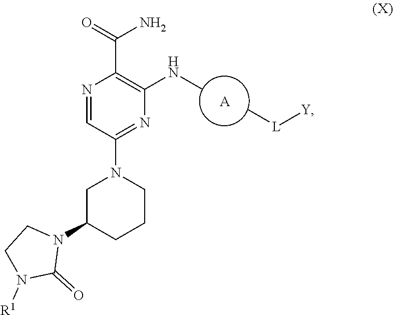
or a pharmaceutically acceptable salt thereof, wherein:
R¹ is C₁₋₃ alkyl;
ring A is phenyl, 5-6 membered partially or fully unsaturated monocyclic heterocycle, 9-10 membered bicyclic aryl, or 9-10 membered bicyclic heteroaryl, wherein the heterocycle and the bicyclic heteroaryl of ring A each independently have 1-3 heteroatoms independently selected from N, O, or S;
L is —X¹—X²—X³—X⁴—X⁵—;
X¹ is C(O) N(R)—, —N(R)—C(O)—, —(O—CH₂—CH₂)ₘ—, —O(C₆H₄)—, —(O—CH₂—CH₂—CH₂)ₘ—, —C₁₋₅ alkyl-, 7-12 membered spiro bicyclic heterocycloalkyl having 1-3 heteroatoms independently selected from N, O, or S wherein the bicyclic heterocycloalkyl of X¹ is optionally substituted with —CH₃, or 4-6 membered monocyclic heterocycloalkyl having 1-2 heteroatoms independently selected from N, O, or S wherein the monocyclic heterocycloalkyl of X is optionally substituted with —CH₃;
X² is a bond —(O—CH₂—CH₂)ₙ—, (CH₂—CH₂—O)ₙ—, —N(R)—C(O)—, —NR)—, —C(O)—, —C₁₋₅ alkyl-, 4-6 membered monocyclic cycloalkyl, or 4-6 membered monocyclic heterocycloalkyl having 1-2 heteroatoms independently selected from N, O, or S;
X³ is a bond, —C₁₋₄ alkyl-, —C≡C—, 4-6 membered cycloalkyl, —N(R)—, —(O—CH₂—CH₂)ₚ—, —(CH₂—CH₂—O)ₚ—, 4-6 membered heterocycloalkyl having 1-2 heteroatoms independently selected from N, O, or S wherein the heterocycloalkyl is optionally substituted with —CH₃ or —N(R)—;
X⁴ is a bond, —CH₂—CH₂—N(R)—, —N(R)—, —C₁₋₄ alkyl-, —(O—CH₂—CH₂—CH₂)ₘ—, a 5-6 membered saturated, partially unsaturated, or fully unsaturated carbocycle, or a 5-6 membered saturated, partially unsaturated, or fully unsaturated heterocycle having 1-3 heteroatoms independently selected from N, O, or S;
X⁵ is a bond, —C₁₋₄ alkyl-, —N(R)—, or —C(O)—N(R)—;
each R is independently —H or —C₁₋₃ alkyl;
each of m, n, and p is independently an integer from 1 to 3; and
Y is 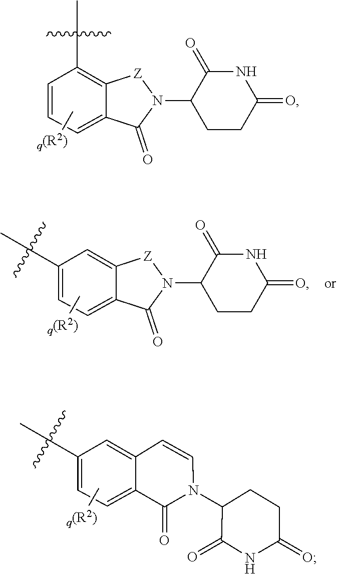
wherein:
each R² is independently halo or C₁₋₄ alkyl;
each Z is —C(R^{A})₂— or —C(O)—;
each R^{A} is independently —H or C₁₋₄ alkyl; and
q is 0, 1, or 2.

108. The compound of claim 107, wherein R¹ is methyl, ethyl, or propyl.

109. The compound of claim 107, ring A is selected from

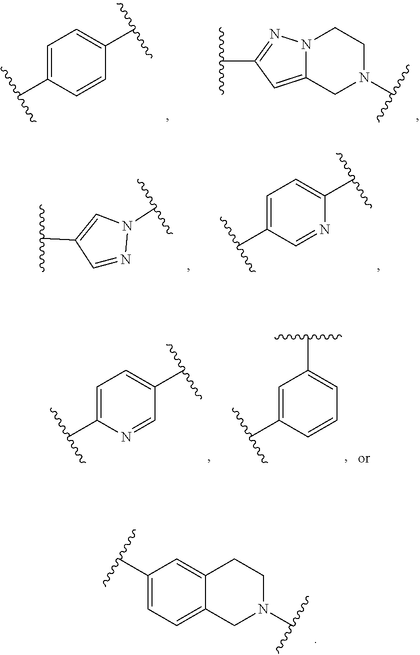

110. The compound of claim 107, wherein at least one of X¹, X², and X⁵ is —C(O)—N(R)— or —CH₂—.

111. The compound of claim 107, wherein X¹ is —C₁₋₅ alkyl-; 7-12 membered spiro bicyclic heterocycloalkyl having 1-3 heteroatoms independently selected from N, O, or S wherein the heterocycloalkyl is optionally substituted with —CH₃; or 4-6 membered heterocycloalkyl having 1-2 heteroatoms independently selected from N, O, or S wherein the heterocycloalkyl is optionally substituted with —C₃.

112. The compound of claim 107, wherein:
X¹ is —CH₂—, —C(O)—, —C(O)—N(R)˜, —(O—CH₂—CH₂—CH₂)ₘ—, 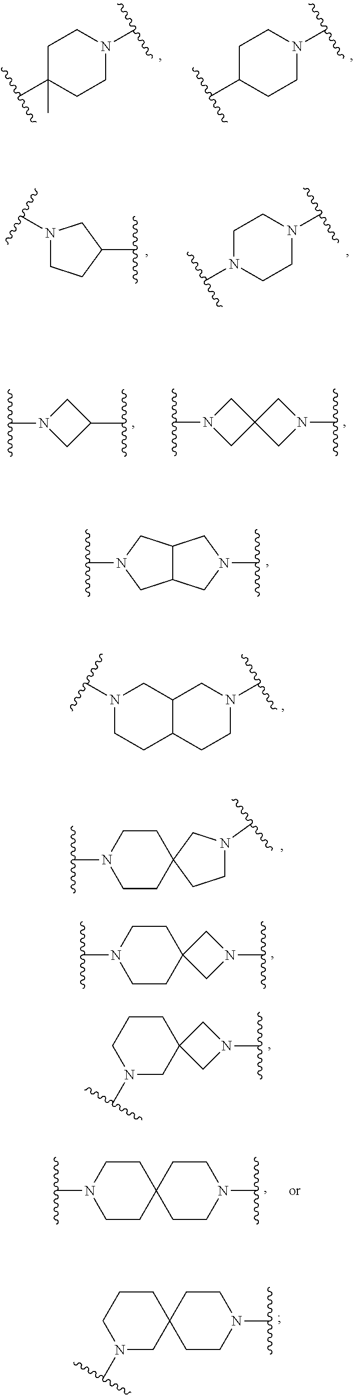
and
when X¹ is —(O—CH₂—CH₂—CH₂)ₘ—, m is 1 and X² is —C(O)—N(R)—.

113. The compound of claim 107, wherein X² is —(O—CH₂—CH₂)ₙ—, —(CH₂—CH₂—O)ₙ—, or —C₁₋₅ alkyl-.

114. The compound of claim 107, wherein X² is a bond, —C(O)—, —CH₂—, —CH₂—CH₂—, —CH₂—CH₂—CH₂—,

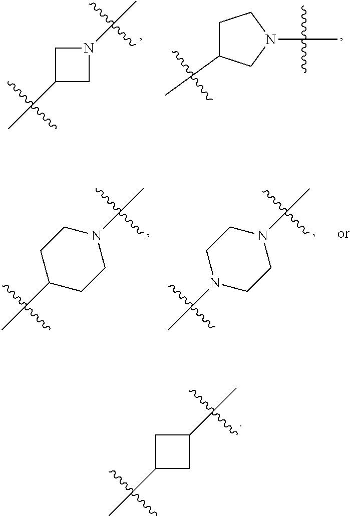 [SMILES: *C1CN(*)C1 ; *C1CCN(*)C1 ; *C1CCN(*)CC1 ; *N1CCN(*)CC1 ; *C1CC(*)C1]

115. The compound of claim 107, wherein X³ is a bond, —C≡C—, —N(R)—, —C₁₋₄ alkyl-, 4-6 membered cycloalkyl, 4-6 membered heterocycloalkyl having 1-2 heteroatoms independently selected from N, O, or S wherein the heterocycloalkyl is optionally substituted with —CH₃.

116. The compound of claim 107, wherein X³ is

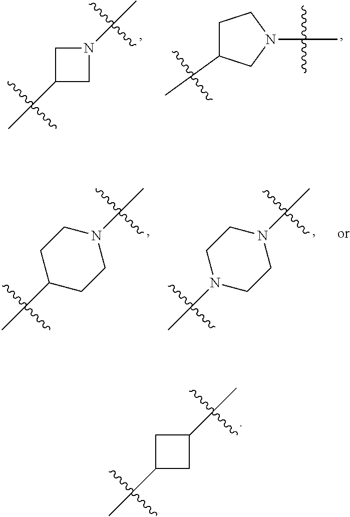 [SMILES: *C1CN(*)C1 ; *C1CCN(*)C1 ; *C1CCN(*)CC1 ; *N1CCN(*)CC1 ; *C1CC(*)C1]

117. The compound of claim 107, wherein X⁴ is a bond, —C₁₋₄ alkyl-, —(O—CH₂—CH₂—CH₂)ₘ—, or 5-6 membered saturated, partially unsaturated, or fully unsaturated carbocycle having 0-3 heteroatoms independently selected from N, O, or S.

118. The compound of claim 107, wherein X⁴ is a bond, 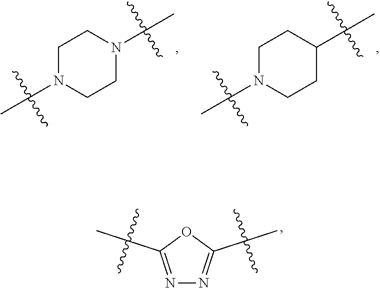 [SMILES: *N1CCN(*)CC1 ; *C1CCN(*)CC1 ; *c1nnc(*)o1]
—CH₂—, —CH₂—CH₂—N(R)—, or —N(R)—.

119. The compound of claim 107, wherein X⁵ is a bond, —C₁₋₄ alkyl-, —N(R)—, or —C(O)—N(R)—.

120. The compound of claim 107, wherein L is

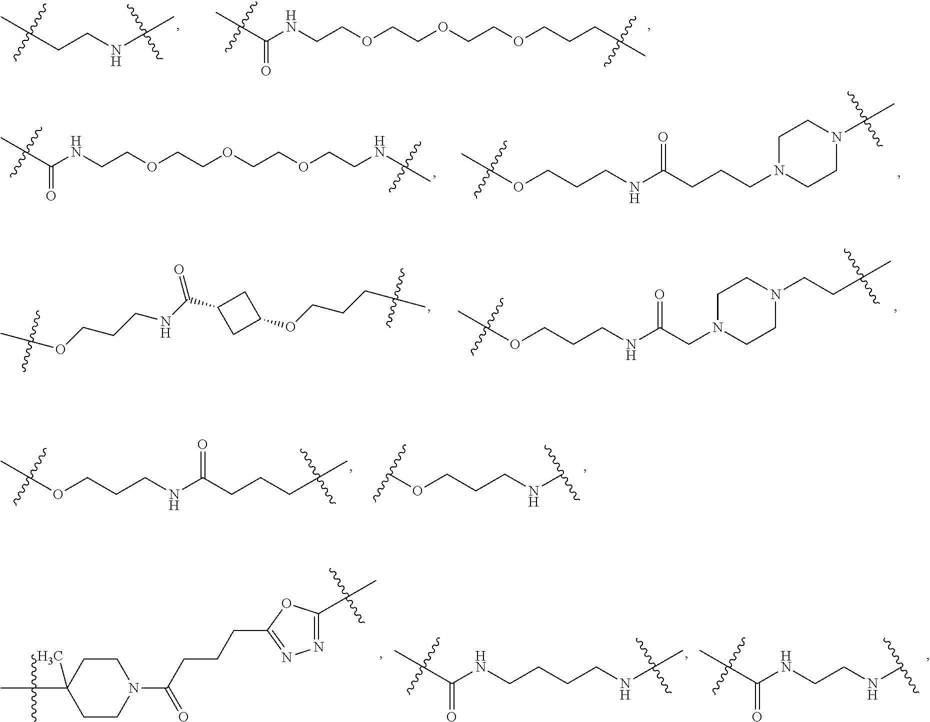

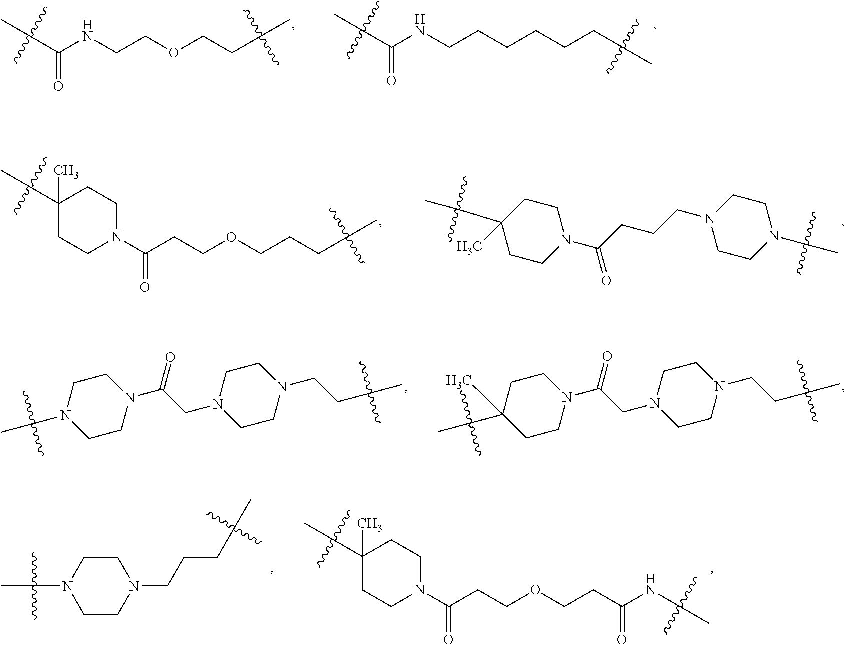

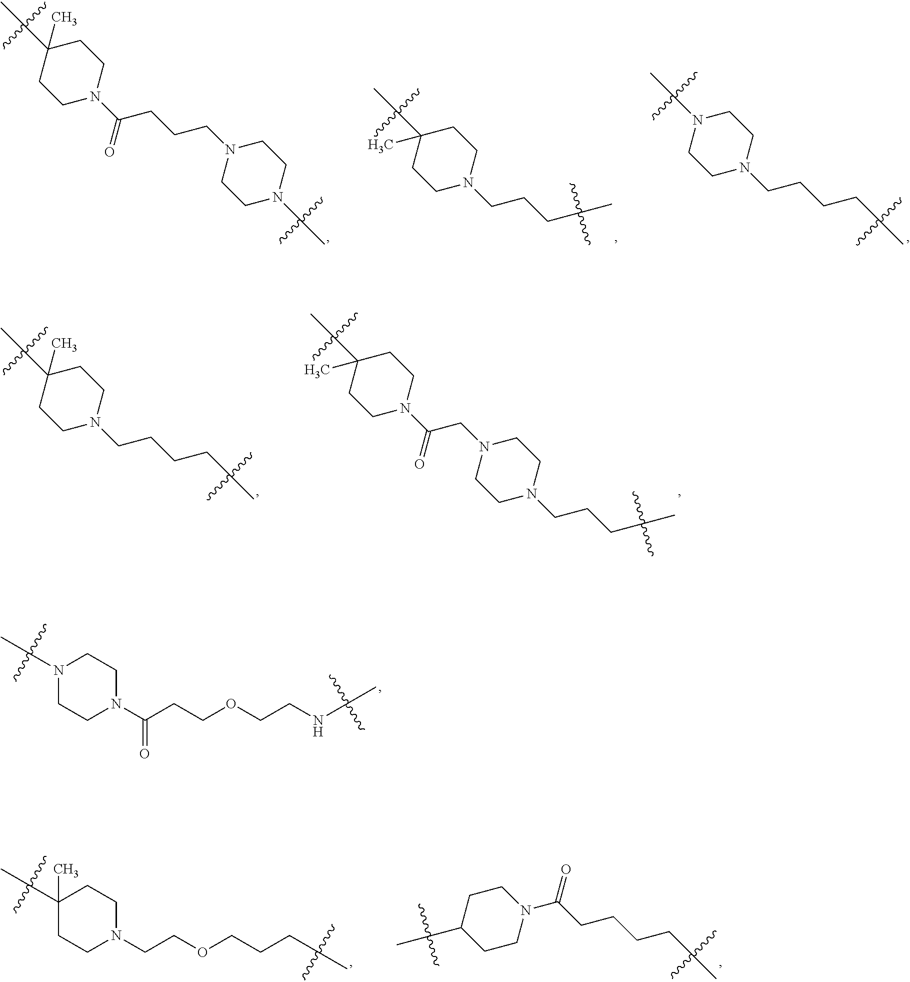

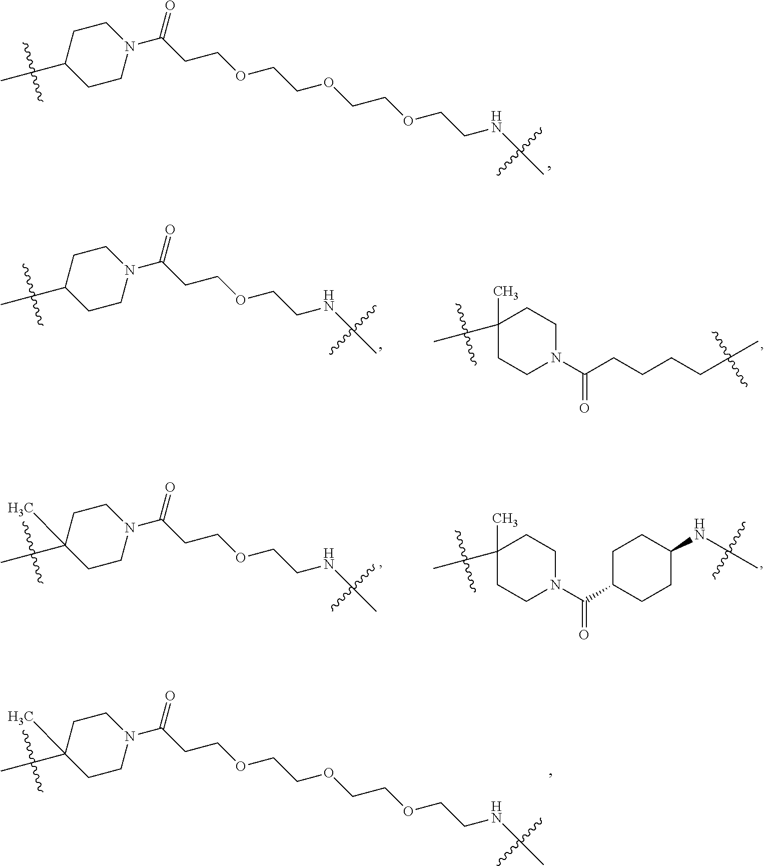 [SMILES: *NCCOCCOCCOCCC(=O)N1CCC(*)CC1 ; *NCCOCCC(=O)N1CCC(*)CC1 ; *CCCCC(=O)N1CCC(*)(C)CC1 ; *NCCOCCC(=O)N1CCC(*)(C)CC1 ; *N[C@H]1CC[C@H](C(=O)N2CCC(*)(C)CC2)CC1 ; *NCCOCCOCCOCCC(=O)N1CCC(*)(C)CC1]

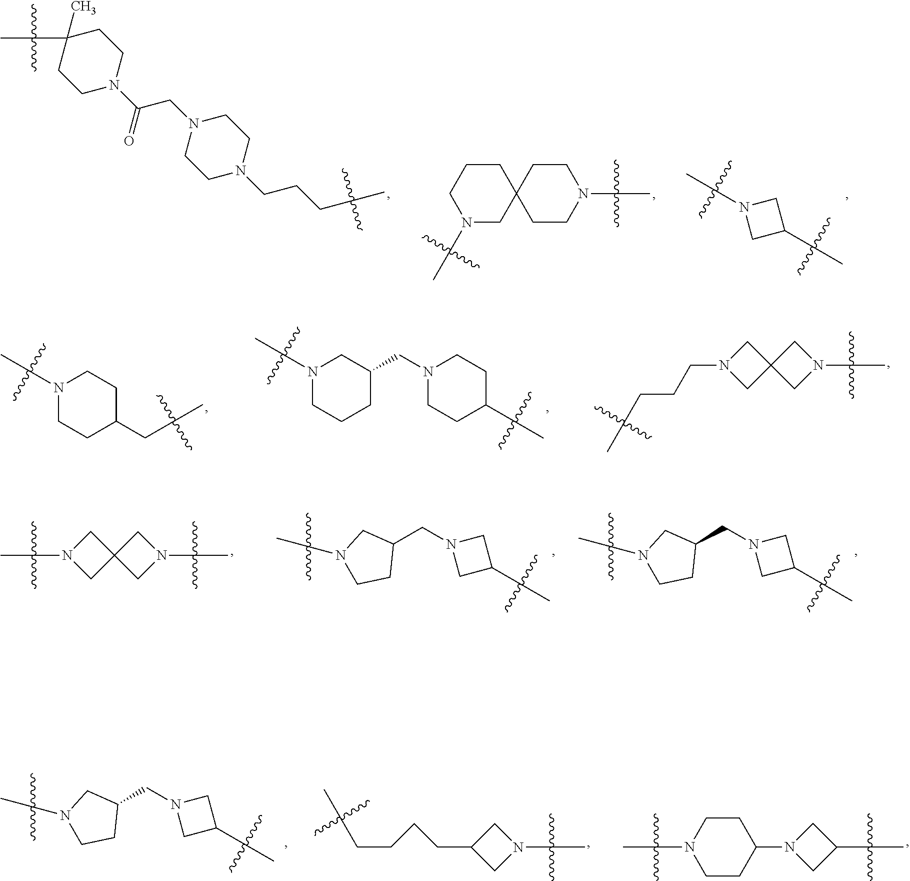

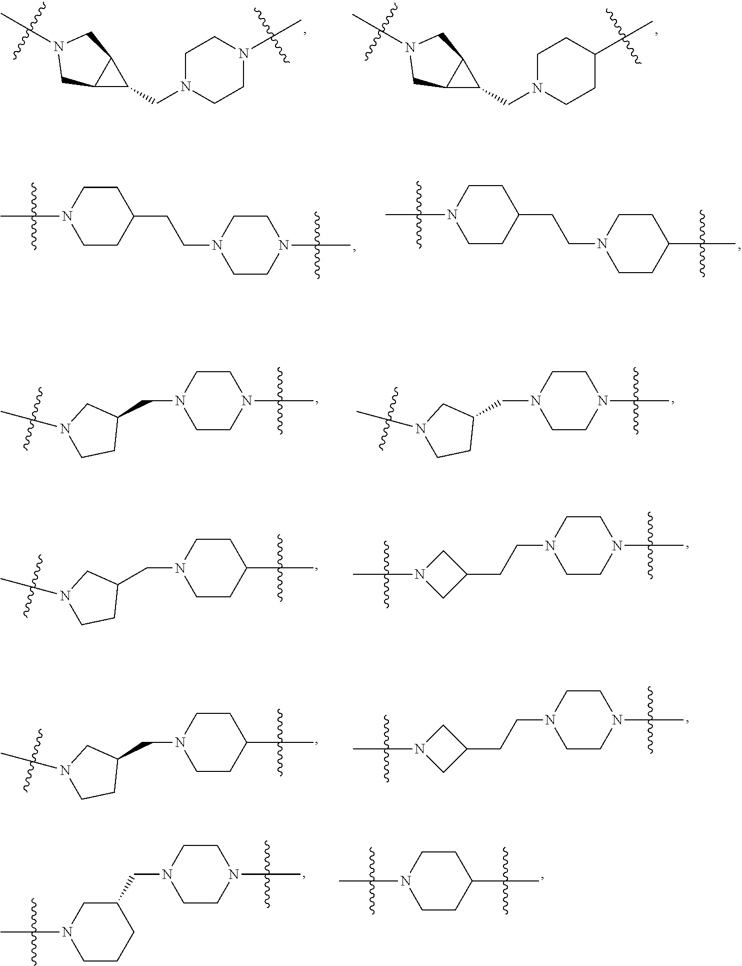

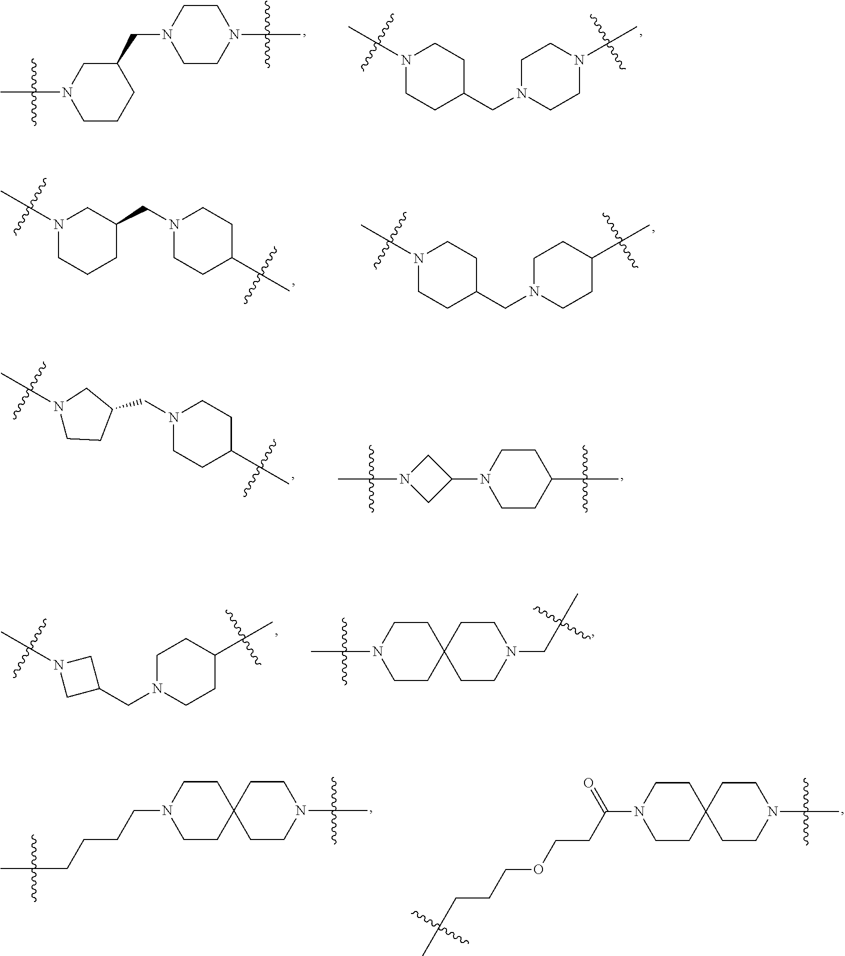

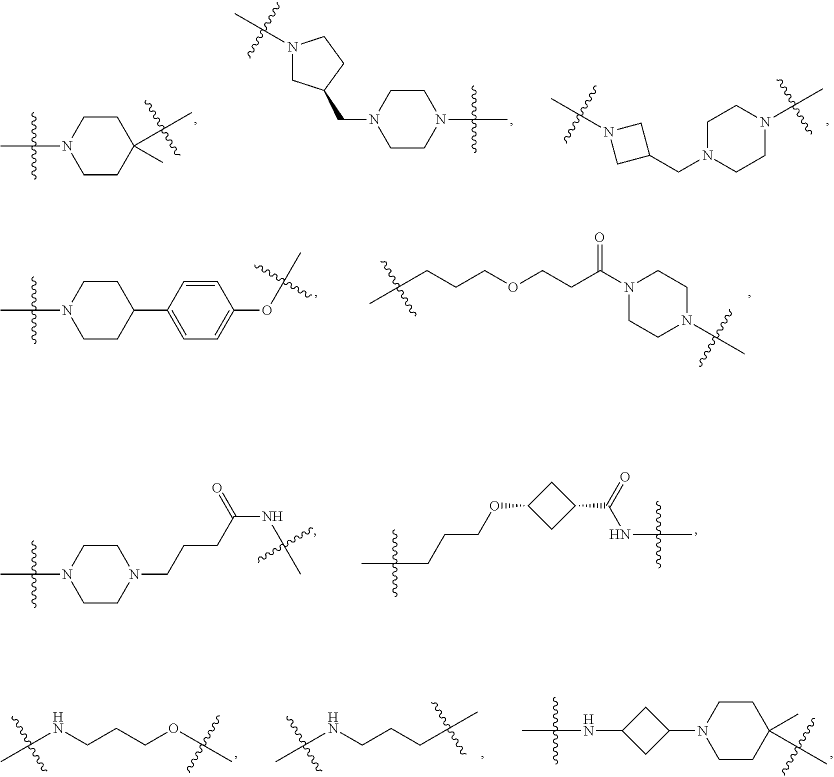

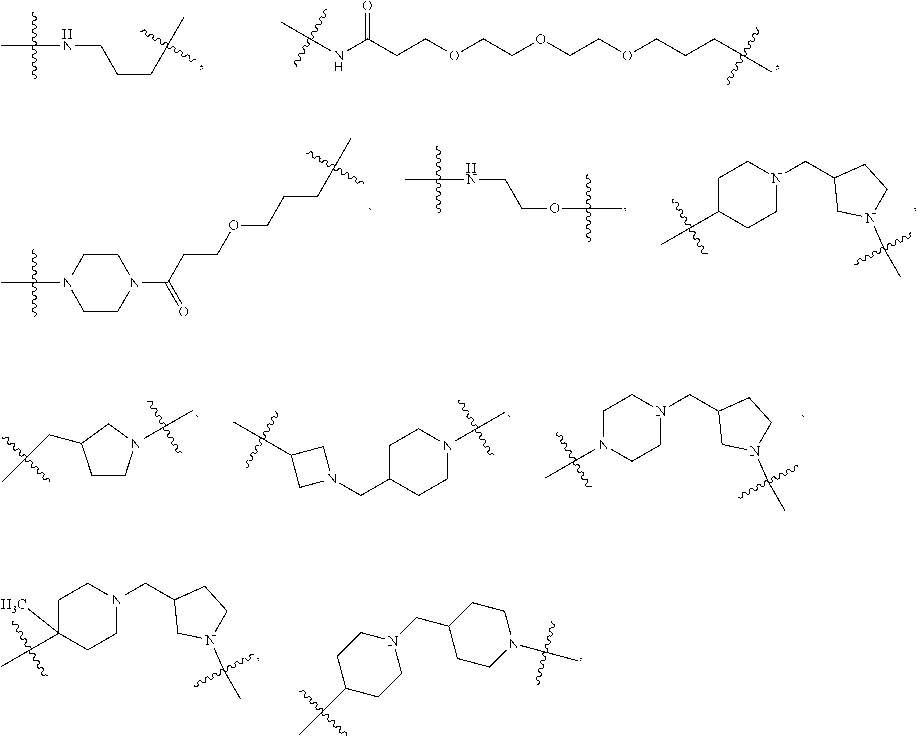

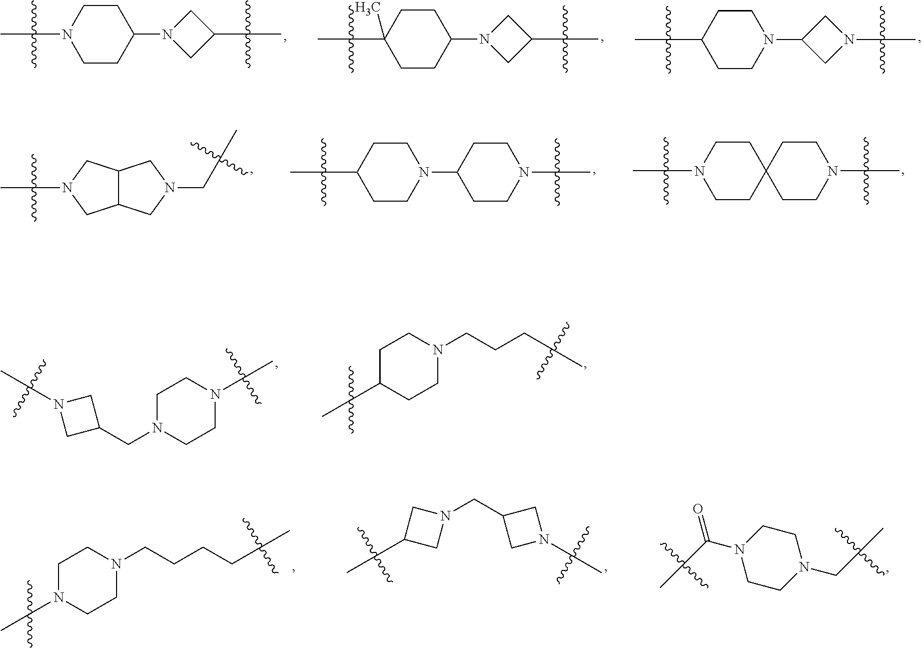

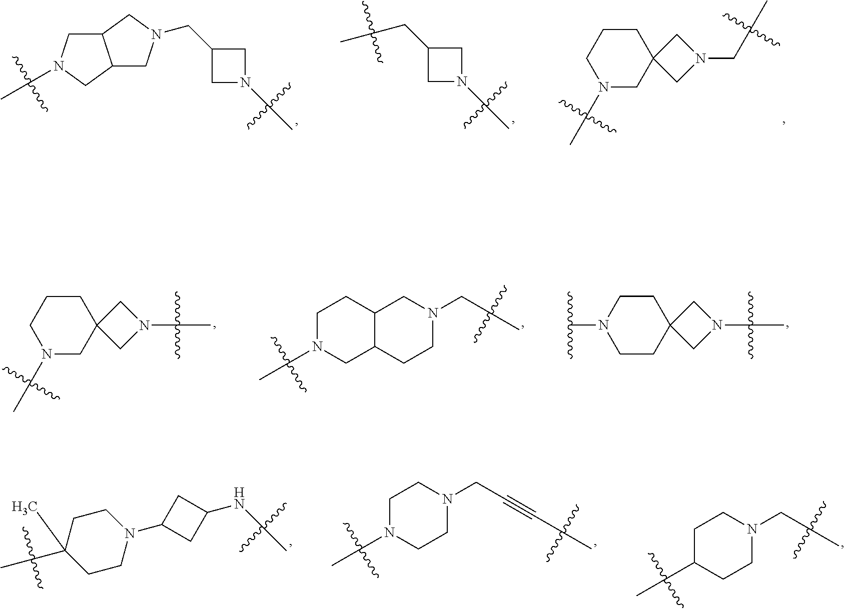

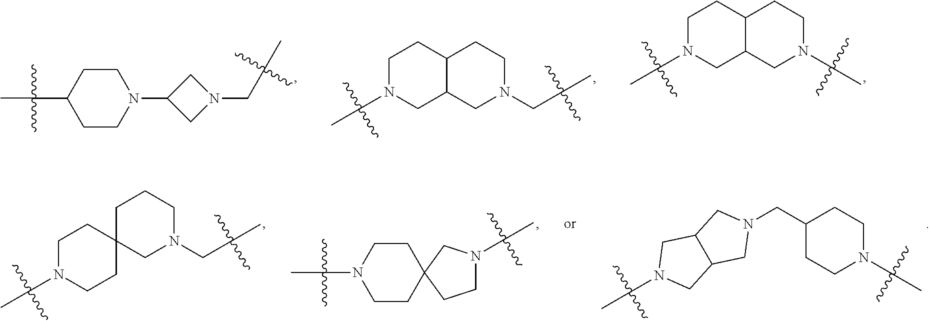 [SMILES: *CN1CC(N2CCC(*)CC2)C1 ; *CN1CCC2CCN(*)CC2C1 ; *N1CCC2CCN(*)CC2C1 ; *CN1CCCC2(CCN(*)CC2)C1 ; *N1CCC2(CC1)CCN(*)C2 ; *N1CCC(CN2CC3CN(*)CC3C2)CC1]

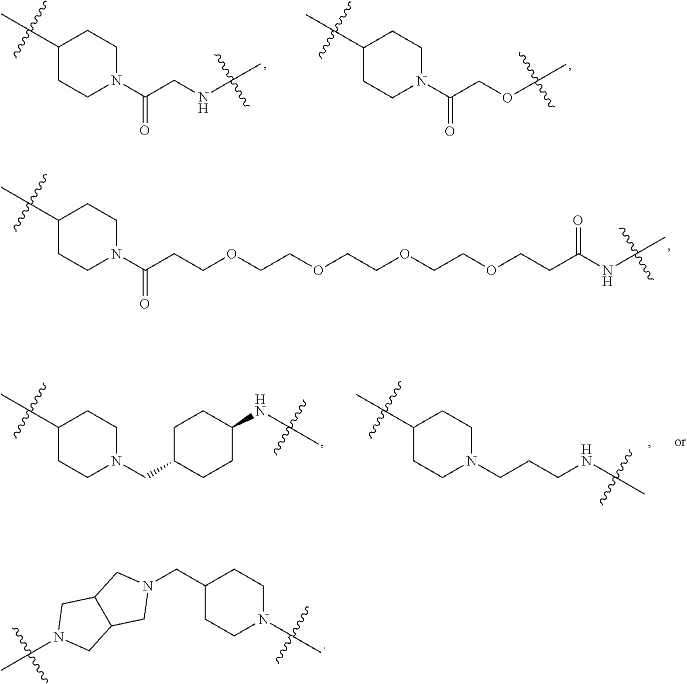 [SMILES: *NCC(=O)N1CCC(*)CC1 ; *OCC(=O)N1CCC(*)CC1 ; *NC(=O)CCOCCOCCOCCOCCC(=O)N1CCC(*)CC1 ; *N[C@H]1CC[C@H](CN2CCC(*)CC2)CC1 ; *NCCCN1CCC(*)CC1 ; *N1CCC(CN2CC3CN(*)CC3C2)CC1]

121. The compound of claim 107, wherein q is 0, or q is 1 and R² is —F.

122. The compound of claim 107, wherein Y is

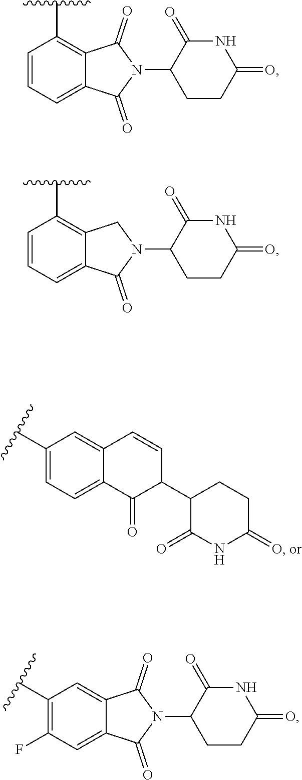

123. The compound of claim 107, wherein the compound is a compound of Formula (I): 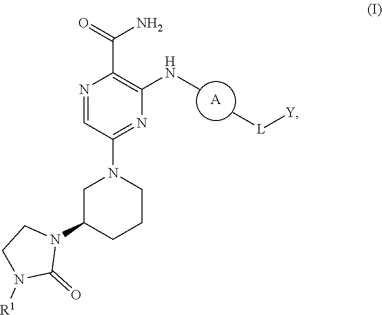
or a pharmaceutically acceptable salt thereof, wherein: R¹; ring A; X¹; X²; X⁴; X⁵; R; each of m, n, and p; each R²; each Z; each R^{A}; and q are as defined in claim 1;
X³ is a bond, —C₁₋₄ alkyl-, 4-6 membered cycloalkyl, —N(R)—, —(O—CH₂—CH₂)ₚ—, —(CH₂—CH₂—O)ₚ—, 4-6 membered heterocycloalkyl having 1-2 heteroatoms independently selected from N, O, or S wherein the heterocycloalkyl is optionally substituted with —CH₃; and
Y is

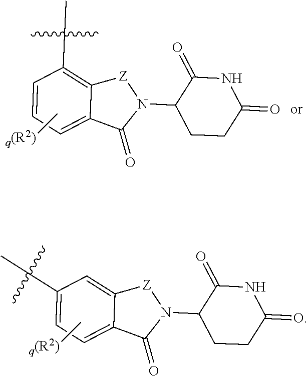

124. The compound of claim 123, wherein the compound is a compound of Formula (I-A), Formula (I-B), Formula (II), Formula (II-A), Formula (II-B): 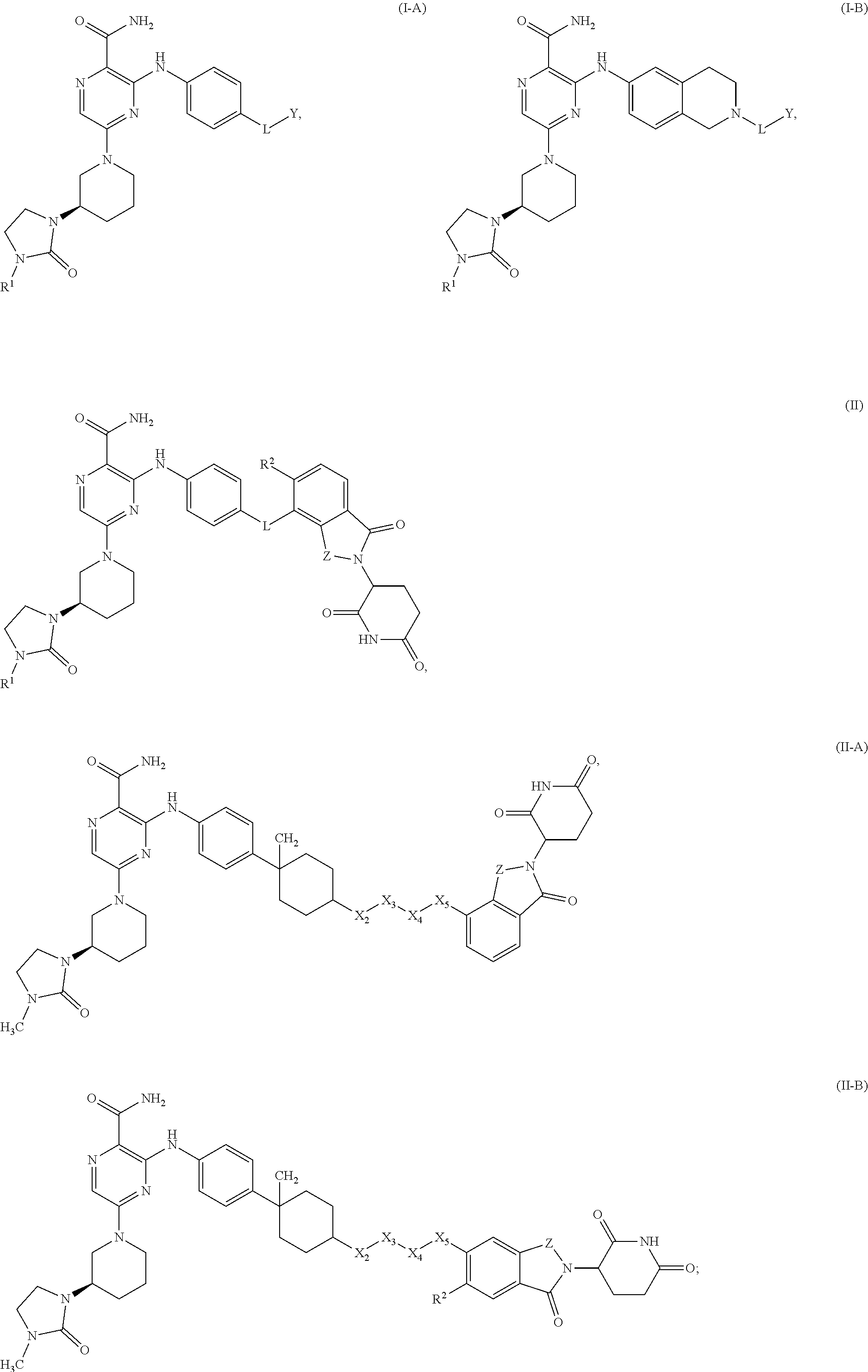
or a pharmaceutically acceptable salt thereof.

125. The compound of claim 107, wherein the compound is a compound of Formula (III): 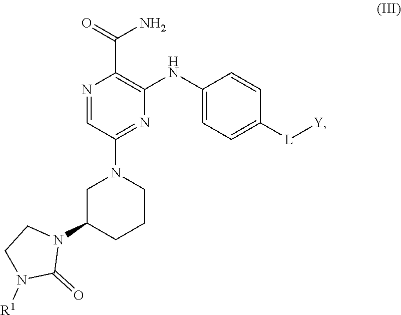
or a pharmaceutically acceptable salt thereof, wherein:
R¹, X¹, X², and X³ are as defined in claim 17;
L is —X¹—X²—X³—; and
Y is 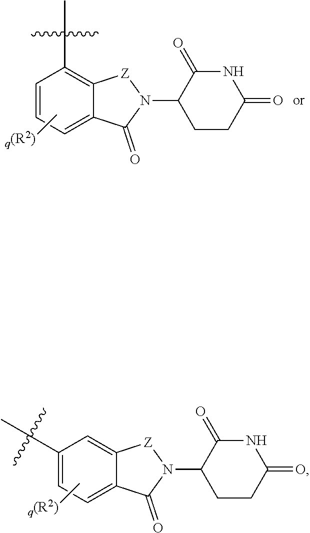
wherein:
each R² is independently halo or C₁₋₄ alkyl;
each Z is —C(R^{A})₂— or —C(O)—;
each R^{A} is independently —H; and
q is 0, 1, or 2.
